(19) United States
(12) Patent Application Publication
(10) Pub. No.: US 2022/0411783 A1
(43) Pub. Date: Dec. 29, 2022

(54) METHOD FOR EXTRACTING NUCLEI OR WHOLE CELLS FROM FORMALIN-FIXED PARAFFIN-EMBEDDED TISSUES

(71) Applicants: The Broad Institute, Inc., Cambridge, MA (US); Massachusetts Institute of Technology, Cambridge, MA (US); The General Hospital Corporation, Boston, MA (US)

(72) Inventors: Aviv Regev, Cambridge, MA (US); Cristin McCabe, Cambridge, MA (US); Eugene Drokhlyansky, Cambridge, MA (US); Alexandre Melnikov, Cambridge, MA (US); Orit Rozenblatt-Rosen, Cambridge, MA (US); Christopher S. Smillie, Cambridge, MA (US); Ramnik J. Xavier, Cambridge, MA (US); Orr Ashenberg, Cambridge, MA (US); Gökcen Eraslan, Cambridge, MA (US); Hattie Chung, Cambridge, MA (US)

(21) Appl. No.: 17/284,542

(22) PCT Filed: Oct. 11, 2019
(86) PCT No.: PCT/US2019/055894

§ 371 Date: Apr. 12, 2021

Related U.S. Application Data
(60) Provisional application No. 62/745,259, filed on Oct. 12, 2018.
(60) Provisional application No. 62/813,634, filed on Mar. 4, 2019.
(60) Provisional application No. 62/829,402, filed on Apr. 4, 2019.
(60) Provisional application No. 62/887,339, filed on Aug. 15, 2019.
(60) Provisional application No. 62/890,971, filed on Aug. 23, 2019.

(51) Int. Cl. C12N 15/10; C12N 5/00; G01N 1/28; G01N 1/34; G01N 1/44

(52) U.S. Cl. CPC C12N 15/1017; C12N 5/0081; G01N 15/14; G01N 1/34; G01N 1/44; G01N 1/286

(57) ABSTRACT

The subject matter disclosed herein is generally directed to isolating single cells and nuclei from tissue samples for use in the analysis of single cells from archived biological samples. The subject matter disclosed herein is directed to isolating single cells and nuclei from formalin-fixed paraffin-embedded (FFPE) tissues. The subject matter disclosed herein is also directed to isolating single nuclei that preserve ribosomes or ribosomes and rough ER from frozen tissues. The subject matter disclosed herein is also directed to therapeutic targets, diagnostic targets and methods of screening for modulating agents.

### CROSS-REFERENCE TO RELATED APPLICATIONS

This application claims the benefit of U.S. Provisional Application No. 62/745,259, filed Oct. 12, 2018; U.S. Provisional Application No. 62/813,634, filed Mar. 4, 2019; U.S. Provisional Application No. 62/829,402, filed Apr. 4, 2019; U.S. Provisional Application No. 62/887,339, filed Aug. 15, 2019; and U.S. Provisional Application No. 62/890,971, filed Aug. 23, 2019. The entire contents of the above-identified applications are hereby fully incorporated herein by reference.

### STATEMENT REGARDING FEDERALLY SPONSORED RESEARCH

This invention was made with government support under Grant No.(s) DK043351, DK114784 and DK117263 awarded by the National Institutes of Health. The government has certain rights in the invention.

### REFERENCE TO AN ELECTRONIC SEQUENCE LISTING

The contents of the electronic sequence listing (BROD_3900_ST25.txt”; Size is 5,073 bytes and it was created on Oct. 11, 2019) is herein incorporated by reference in its entirety.

### TECHNICAL FIELD

The subject matter disclosed herein is generally directed to methods of single nuclei sequencing. The subject matter disclosed herein is also directed to isolating single cells and nuclei from frozen and formalin-fixed paraffin-embedded (FFPE) tissues for use in the analysis of single cells from archived biological samples. The subject matter disclosed herein is also directed to therapeutic targets, diagnostic targets and methods of screening for modulating agents.

### BACKGROUND

Single cell methods (e.g., single cell RNA-Seq) has greatly extended our understanding of heterogeneous tissues, including the CNS (A. Zeisel et al., Brain structure. Cell types in the mouse cortex and hippocampus revealed by single-cell RNA-seq. Science 347, 1138-1142 (2015); S. Darmanis et al., A survey of human brain transcriptome diversity at the single cell level. Proc Natl Acad Sci USA 112, 7285-7290 (2015); J. Shin et al., Single-Cell RNA-Seq with Waterfall Reveals Molecular Cascades underlying Adult Neurogenesis. Cell Stem Cell 17, 360-372 (2015); B. Tasic et al., Adult mouse cortical cell taxonomy revealed by single cell transcriptomics. Nat Neurosci 19, 335-346 (2016); D. Usoskin et al., Unbiased classification of sensory neuron types by large-scale single-cell RNA sequencing. Nat Neurosci 18, 145-153 (2015); E. R. Thomsen et al., Fixed single-cell transcriptomic characterization of human radial glial diversity. Nat Methods 13, 87-93 (2016)), and is reshaping the concept of cell type and state. Formalin-fixed paraffin-embedded (FFPE) tissues are available for archival tissues, provide for easy storage and shipping, are available for rare diseases, and have well documented pathology. However, analyzing single cells from FFPE tissues has been challenging. For example, FFPE samples may have damaged cellular structures, low input and degraded/fragmented RNA, and the samples are cross linked. Thus, there is a need for improved devices and methods to allow for understanding heterogeneous tissues and cell populations present in FFPE samples.

Despite its central role in intestinal function and health, our understanding of the ENS is limited due to longstanding technical challenges; most of our knowledge to date is based on immunohistochemistry with a limited number of known markers. Because the ENS is dispersed among other cell types within the intestine (e.g., myocytes and fibroblasts), enteric neurons are rare in any sample. Moreover, they are exceptionally challenging to isolate and study with genomic tools. Finally, most work on the ENS to date has been performed in rodent models with relatively few human studies (13). Single cell methods currently are not able to be used to analyze tissues from the ENS. Thus, there is a need for improved devices and methods to allow for understanding heterogeneous tissues and cell populations, such as the ENS. Moreover, treatment of diseases associated with the ENS are needed and require new biomarkers, methods of screening and therapeutic targets.

### SUMMARY

In certain example embodiments, the present invention provides for methods of isolating nuclei or whole cells from tissue samples (e.g., frozen or FFPE). In further example embodiments, the invention provides for a method of single cell sequencing comprising: extracting nuclei from a tissue sample under conditions that preserve the nuclear membranes, ribosomes and/or rough endoplasmic reticulum (ER); sorting single nuclei into separate reaction vessels; extracting RNA from the single nuclei; generating a cDNA library; and sequencing the library, whereby gene expression data from single cells is obtained. In further example embodiments, the invention provides for a method of single cell sequencing comprising: extracting whole cells from a tissue sample under conditions that preserve the cell membranes; sorting single cells into separate reaction vessels; extracting RNA from the single cells; generating a cDNA library; and sequencing the library, whereby gene expression data from single cells is obtained. In some embodiments, the reaction vessels may be single cell droplets.

In one aspect, the present invention provides for a method of recovering nuclei or whole cells from a formalin-fixed paraffin-embedded (FFPE) tissue comprising: dissolving paraffin from a FFPE tissue sample in a solvent, preferably the solvent is selected from the group consisting of xylene and mineral oil, wherein the tissue is dissolved at a temperature between 4 C to 90 C, preferably room temperature (20 to 25 C) for recovering whole cells and 90 C for recovering nuclei; rehydrating the tissue using a gradient of ethanol from 100% to 0% ethanol (EtOH); transferring the rehydrated tissue to a volume of a first buffer comprising a buffering agent, a detergent and an ionic strength between 100 mM and 200 mM, optionally the first buffer comprises protease inhibitors or proteases and/or BSA; chopping or dounce homogenizing the tissue in the buffer; and removing debris by filtering and/or FACS sorting.

In certain embodiments, the method further comprises isolating nuclei or cell types by FACS sorting.

In certain embodiments, dissolving paraffin from a FFPE tissue sample, comprises incubating at least one time in xylene, at room temperature (RT), for about 10 minutes each, and wherein xylene is removed at each change. In certain embodiments, the method further comprises washing the tissue at least two times with xylene for about 10 min each, wherein the washes are performed at room temperature (RT), 90 C, or at least one time at room temperature (RT) and at least one time at 90 C, wherein xylene is removed at each change.

In certain embodiments, dissolving paraffin from a FFPE tissue sample, comprises incubating at least twice in about 5 ml xylene per 30-100 mg FFPE tissue sample, at room temperature, for about 10 minutes each, wherein xylene is removed at each change. In certain embodiments, the method further comprises washing the tissue with xylene at 37 C for about 10 min. In certain embodiments, the method further comprises cutting the tissue into two or more pieces and washing at least one piece of the tissue with xylene at 37 C for about 10 min.

In certain embodiments, dissolving paraffin from a FFPE tissue sample, comprises incubating at least three times in xylene, at room temperature, for about 10 minutes each, and wherein xylene is removed at each change. In certain embodiments, the method further comprises washing the tissue three additional times with xylene for about 10 min each, wherein the first wash is at room temperature and the second and third washes are at 90 C, and wherein xylene is removed at each change.

In certain embodiments, rehydrating the tissue comprises a step gradient of ethanol (EtOH) and the tissue is incubated between 1 to 10 minutes at each step. In certain embodiments, the step gradient comprises incubating the tissue for about 2 minutes each in successive washes of 95%, 75%, and 50% ethanol (EtOH).

In certain embodiments, after rehydrating the tissue the method further comprises placing the tissue samples on ice or on a device capable of maintaining the tissue between 4 and 10 C, wherein all subsequent steps are performed at a temperature between 4 and 10 C.

In certain embodiments, after the step of dissolving paraffin from the tissue or rehydrating the tissue the method further comprises dividing the tissue, preferably in half.

In certain embodiments, the first buffer comprises a detergent selected from the group consisting of NP40, CHAPS and Tween-20. In certain embodiments, the NP40 concentration is about 0.2%. In certain embodiments, the Tween-20 concentration is about 0.03%. In certain embodiments, the CHAPS concentration is about 0.49%. In certain embodiments, the first buffer is selected from the group consisting of CST, TST, NST and NSTnPo.

In certain embodiments, after the step of chopping or dounce homogenizing the method further comprises centrifuging, preferably, the sample is centrifuged at about 500 g for about 5 min, and resuspending the sample in a second buffer comprising a buffering agent and an ionic strength between 100 mM and 200 mM, optionally the second buffer comprises protease inhibitors. In certain embodiments, the second buffer is ST, optionally comprising protease inhibitors.

In certain embodiments, the sample is filtered through a 40 uM filter. In certain embodiments, the method further comprises washing the filtered sample in the first buffer. In certain embodiments, the method further comprises filtering the sample through a 30 uM filter.

In certain embodiments, after the step of chopping or dounce homogenizing the method further comprises adding an additional 2 volumes of the first buffer (3 volumes total) and filtering the sample through a 40 uM filter. In certain embodiments, the method further comprises adding an additional three volumes of the first buffer (6 volumes total), centrifuging, preferably, the sample is centrifuged at about 500 g for about 5 min, and resuspending the sample in a second buffer comprising a buffering agent and an ionic strength between 100 mM and 200 mM, optionally the second buffer comprises protease inhibitors. In certain embodiments, the second buffer is ST, optionally comprising protease inhibitors.

In certain embodiments, the method further comprises reversing cross-linking in the tissue sample before or during any step of the method. In certain embodiments, reversing cross-linking comprises proteinase digestion. In certain embodiments, the proteinase is proteinase K or a cold-active protease.

In certain embodiments, the method further comprises adding a reagent that stabilizes RNA to the tissue sample before or during any step of the method.

In certain embodiments, the method further comprises lysing recovered cells or nuclei and performing reverse transcription. In certain embodiments, the reverse transcription is performed in individual reaction vessels. In certain embodiments, the reaction vessels are wells, chambers, or droplets.

In certain embodiments, the method further comprises performing single cell, single nucleus or bulk RNA-seq, DNA-seq, ATAC-seq, or ChIP on the recovered nuclei or whole cells.

In certain embodiments, the method further comprises staining the recovered cells or nuclei. In certain embodiments, the stain comprises ruby stain.

In certain embodiments, single cells or nuclei are enriched by FACS or magnetic-activated cell sorting (MACS). The nuclei or cells of any method described herein may further be detectable by a fluorescent signal, whereby individual nuclei or cells may be further sorted. The single nuclei or cells may be immunostained with an antibody with specific affinity for an intranuclear protein or cell surface protein. The antibody may be specific for NeuN. The nuclei may be stained with a nuclear stain. The nuclear stain may comprise DAPI, Ruby red, trypan blue, Hoechst or propidium iodine. In certain embodiments, nuclei can be labeled with ruby dye (Thermo Fisher Scientific, Vybrant DyeCycle Ruby Stain, #V-10309) added to the resuspension buffer at a concentration of 1:800.

In certain embodiments, the tissue sample is obtained from a subject suffering from a disease. In certain embodiments, the disease is cancer, a neurological disease, autoimmune disease, infection, or metabolic disease. The heterogeneous population of cells may be derived from a section of a tissue or a tumor from a subject. The section may be obtained by microdissection. The tissue may be nervous tissue. The nervous tissue maybe isolated from the brain, spinal cord or retina.

In another aspect, the present invention provides for a method of recovering nuclei and attached ribosomes from a tissue sample comprising: chopping the tissue sample at between 0-4° C. in a nuclear extraction buffer comprising Tris buffer, a detergent and salts; and filtering the sample through a filter between 30-50 uM, preferably 40 uM, and optionally washing the filter with fresh nuclear extraction buffer, wherein the nuclei are present in the supernatant passed through the filter. In certain embodiments, the nuclear extraction buffer comprises 10-20 mM Tris, about 0.49% CHAPS, a salt concentration having an ionic strength of 100-250 mM, and about 0.01% BSA, whereby nuclei are recovered that have a preserved nuclear envelope and ribosomes. In certain embodiments, the nuclear extraction buffer is buffer CST. In certain embodiments, the nuclear extraction buffer comprises 10-20 mM Tris, about 0.03% Tween-20, a salt concentration having an ionic strength of 100-250 mM, and about 0.01% BSA, whereby nuclei are recovered that have a preserved nuclear envelope, rough ER and ribosomes. In certain embodiments, the nuclear extraction buffer is buffer TST. In certain embodiments, the salts comprise 146 mM NaCl, 1 mM CaCl₂, and 21 mM MgCl₂. In certain embodiments, chopping comprises chopping with scissors for 1-10 minutes.

In certain embodiments, nuclei from specific cell types are genetically modified to express a detectable label on the nuclear membrane and the method further comprises enriching nuclei from the specific cell types using the detectable label. In certain embodiments, the method further comprises staining the recovered nuclei. In certain embodiments, the stain comprises ruby stain. In certain embodiments, the nuclei are sorted into discrete volumes by FACS.

In certain embodiments, the method further comprises pelleting the nuclei and resuspending the nuclei in a second buffer consisting of Tris buffer and salts. In certain embodiments, the second buffer is buffer ST.

In certain embodiments, the method further comprises generating a single nuclei barcoded library for the recovered nuclei, wherein the nucleic acid from each nuclei is labeled with a barcode sequence comprising a cell of origin barcode, optionally the barcode sequence includes a cell of origin barcode and a unique molecular identifier (UMI). In certain embodiments, RNA and/or DNA is labeled with the barcode sequence. In certain embodiments, the library is an RNA-seq, DNA-seq, and/or ATAC-seq library. In certain embodiments, the method further comprises sequencing the library.

In certain embodiments, the tissue sample is fresh frozen. In certain embodiments, the tissue sample comprises cells originating from the central nervous system (CNS) or enteric nervous system (ENS). In certain embodiments, the tissue sample is obtained from the gut or the brain. In certain embodiments, the tissue sample is obtained from a subject suffering from a disease. In certain embodiments, the tissue sample is treated with a reagent that stabilizes RNA.

In certain embodiments, the discrete volumes are droplets, wells in a plate, or microfluidic chambers.

In another aspect, the present invention provides for a method of treating a disease selected from the group consisting of Hirschsprung's disease (HSCR), inflammatory bowel disease (IBD), autism spectrum disorder (ASD), Parkinson's disease (PD) and schizophrenia in a subject in need thereof comprising administering one or more agents capable of modulating the function or activity of: one or more neurons selected from the group consisting of PEMN1, PEMN2, PIMN1, PIMN2, PIMN3, PIMN4, PIMN5, PIN1, PIN2, PSN and PSVN; or one or more cells functionally interacting with the one or more neurons. In certain embodiments, the one or more cells functionally interacting with the one or more neurons are selected from the group consisting of T cells, dendritic cells (DC), B cells, fibroblasts and adipocytes.

In another aspect, the present invention provides for a method of modulating appetite and energy metabolism in a subject in need thereof comprising administering one or more agents capable of modulating the function or activity of: one or more neurons selected from the group consisting of PIMN4 and PIMN5; or one or more adipose cells functionally interacting with the one or more neurons.

In certain embodiments, the one or more neurons are characterized by expression of one or more markers according to Table 14 or Table 21. In certain embodiments, the one or more agents modulate the expression, activity or function of one or more genes according to Table 14 or Table 21. In certain embodiments, the one or more agents modulate the expression, activity or function of one or more genes selected from the group consisting of: NPY, CGRP, Glutamate, GABA, LEP, VIP, PACAP, Nitric oxide, NOS1, FGF1, PDGF, SLIT2, SLIT3, IL15, IL7, IL12A, PENK, CHAT and TPH2; or NPYR1, CALCRL, GRM8, GABRE, LEPR, VIPR2, GRIA4, GUCY1A3, FGFR1, PDGFRB, ROBO1, ROBO2, IL15R, IL7R, IL12RB1, OPRM1, CHRNE and HTR3A. In certain embodiments, the one or more agents modulate the expression, activity or function of one or more genes selected from the group consisting of: NPY and CGRP; or NPYR1 and CALCRL. In certain embodiments, the one or more agents modulate the expression, activity or function of one or more core transcriptional programs according to Table 23. In certain embodiments, the one or more agents modulate the expression, activity or function of one or more genes of the one or more core transcriptional programs.

In certain embodiments, the one or more agents comprise an antibody, small molecule, small molecule degrader, genetic modifying agent, nucleic acid agent, antibody-like protein scaffold, aptamer, protein, or any combination thereof. In certain embodiments, the genetic modifying agent comprises a CRISPR system, RNAi system, a zinc finger nuclease system, a TALE, or a meganuclease. In certain embodiments, the CRISPR system comprises Cas9, Cas12, or Cas14. In certain embodiments, the CRISPR system comprises a dCas fused or otherwise linked to a nucleotide deaminase. In certain embodiments, the nucleotide deaminase is a cytidine deaminase or an adenosine deaminase. In certain embodiments, the dCas is a dCas9, dCas12, dCas13, or dCas14. In certain embodiments, the nucleic acid agent or genetic modifying agent is administered with a vector. In certain embodiments, the nucleic acid agent or genetic modifying agent is under the control of a promoter specific to a marker gene for the one or more neurons according to Table 14 or Table 21. In certain embodiments, the nucleic acid agent is a nucleotide sequence encoding the one or more genes (e.g., an overexpression vector, a sequence encoding a cDNA of a gene).

In certain embodiments, the one or more agents are administered to the gut.

In another aspect, the present invention provides for a method of detecting one or more cells of the enteric nervous system (ENS) comprising detecting one or more markers according to Table 14-17 or Table 20-22. In certain embodiments, detecting the one or more markers comprises immunohistochemistry.

In another aspect, the present invention provides for a method of screening for agents capable of modulating expression of a transcription program according to Table 23 comprising: administering an agent to a population of cells comprising neurons selected from the group consisting of PEMN1, PEMN2, PIMN1, PIMN2, PIMN3, PIMN4, PIMN5, PIN1, PIN2, PSN and PSVN; and detecting expression of one or more genes in the transcriptional program. In certain embodiments, detecting expression comprises RT-PCR, RNA-seq, single cell RNA-seq, fluorescently labeled probes, or an immunoassay. In certain embodiments, the neurons express one or more reporter genes under control of a promoter specific to the one or more genes in the transcriptional program and detecting comprises detecting the reporter gene.

In another aspect, the present invention provides for a method of identifying gene expression in single cells comprising providing sequencing reads from a single nuclei sequencing library and counting sequencing reads mapping to introns and exons. In certain embodiments, the method further comprises filtering the single nuclei. In certain embodiments, nuclei doublets are removed by filtering. In certain embodiments, nuclei containing ambient RNA or ambient RNA alone is removed by filtering.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Schematic of variables of extracting nuclei from a FFPE tissue block and preparing cDNA.

FIG. 2—Image of nuclei and FACS plot using douncing in the FFPE extraction protocol.

FIG. 3—Image of nuclei and FACS plot using chopping in the FFPE extraction protocol.

FIG. 4—Image of nuclei and FACS plot using 90 C extraction and proteinase K in the FFPE extraction protocol.

FIG. 5—Image of nuclei and FACS plot using 90 C extraction and no proteinase K in the FFPE extraction protocol.

FIG. 6—Image of nuclei and FACS plot using room temperature extraction and proteinase K in the FFPE extraction protocol.

FIG. 7—Image of nuclei and FACS plot using room temperature extraction and no proteinase K in the FFPE extraction protocol.

FIG. 8—Image of nuclei obtained from B16 PDX (patient derived xenograft) using 90 C extraction in the FFPE extraction protocol.

FIG. 9—Image of cells obtained from B16 PDX (patient derived xenograft) using room temperature extraction in the FFPE extraction protocol.

FIG. 10—Image of nuclei obtained from d4mra (patient derived xenograft) using 90 C extraction in the FFPE extraction protocol.

FIG. 11—Image of cells obtained from d4mra (patient derived xenograft) using room temperature extraction in the FFPE extraction protocol.

FIG. 12—Images of nuclei and cells obtained using the FFPE extraction protocol.

FIG. 13—Bioanalyzer electropherograms showing RNA quality (left) and cDNA traces after amplification (right).

FIG. 14—Image of nuclei used for RNA extraction and electropherograms showing cDNA traces with and without heat steps.

FIG. 15—Bioanalyzer electropherogram showing cDNA traces from bulk sorted nuclei.

FIG. 16—Bioanalyzer electropherograms from the samples in Table 5. (xylene sample in rows, oil sample in row 5, and frozen sample in row 8).

FIG. 17—Bioanalyzer electropherograms from the samples extracted with TCL, 5000 nuclei and Xylene RNA control.

FIG. 18—Bioanalyzer electropherograms from a FFPE sample treated at 55 C for 15 minutes using TCL lysis buffer and oil isolation.

FIG. 19—Bioanalyzer electropherograms from xylene extracted total RNA.

FIG. 20—RAISIN RNA-seq captures RNA from intact nuclei and associated ribosomes. (A) Study overview. (B) Neuron nuclei enrichment with reporter mice. Representative histology (left) and FACS (right) of ENS nuclei labelling. Histology and FACS images for all models are in FIG. 24A-C. (C-E) optimization of RAISIN and INNER Cell RNA-seq. (C) Cellular composition of each extraction. Ternary plot showing the proportion of nuclei expressing neuron, glia or neither signature (triangle edges) from each extraction type (dots). Purple, green: published protocols (16, 17). Blue, red: top performing protocols. (n=5,236 GFP+ sorted nuclei across all protocols). (D) RAISIN and INNER Cell RNA-seq isolate nuclei with attached ribosomes and rough ER. Ultra-thin section transmission electron microscopy (TEM) of nuclei extractions from published methods (top) (16, 17) and with RAISIN (bottom left) and INNER Cell (bottom right) methods. (E) Higher exon:intron ratios in RAISIN and INNER Cell methods. Exon:intron ratio (y axis, log₂(ratio)) following snRNA-seq from each preparations in (D). All comparisons significant (Wilcoxon test, p-value<10⁻¹⁰); boxplots: 25%, 50%, and 75% quantiles; error bars: standard deviation (SD). (F) RAISIN RNA-seq is compatible with droplet-based RNA-seq. A t-distributed stochastic neighbor embedding (t-SNE) of RAISIN RNA-seq profiles from mouse colon of 10,889 unsorted RAISINs profiled by droplet-based scRNA-seq and colored by cell type.

FIG. 21—Mouse ENS atlas reveals 24 neuron subsets that vary with circadian phase and colon location. (A-B) Mouse neuron reference map. (A) 24 neuron subsets profiled by RAISIN RNA-seq. t-SNE of 2,447 neuron RAISIN RNA-Seq profiles from mouse colon colored by major putative neuron classes based on post hoc annotation (SOM). (B) Neuron subsets vary by anatomical location and mouse line. Neuron subsets (columns) arranged by transcriptional similarity (dendrogram, top) and annotated with the proportion of cells isolated from each transgenic model (green pie chart) or colon segment (red/blue pie chart). Dot plot shows for select neurotransmitters and neuropeptides (rows), the fraction of cells in each subset (dot size) expressing the synthetic enzyme (top) or respective receptors (bottom) (genes for synthesis and receptors in table 18), and the mean expression level in expressing cells in the subset (dot color). (C,D) Mouse ENS gene expression is affected by circadian rhythm. Distribution of neuron gene expression levels (y axis, log₂(TP10K+1)) of select genes (x axis) that are upregulated at morning (red) or evening (blue) time points in all neurons (C) or at the morning time point in PSN1s and PSN2s (D). (E) Changes in ENS expression along colon length. Mean expression across all neuron subsets (color bar) of significantly DE genes (columns) across colon regions (rows), arranged by location of peak expression from proximal to distal. (F) Revisions to the peristaltic model. Left: current model of the peristaltic circuit (adapted from 13). Right: additions to this model derived from the ENS atlas. (G) The mechanosensitive ion channel Piezo1 is expressed in PIMNs and PEMNs. Distribution of gene expression levels (y axis, log₂(TP10K+1)) across neuron subsets (x axis) for genes in peristaltic model: Htr4 (top), Piezo1 (middle) and Piezo2 (bottom). (II,I) Validation of gene expression in situ. Representative images of smFISH for Calcb and Nmu (G) or Nog and Grp (H), both with Tubb3 immunostaining. Merged channels on right. Inset: example neuron expressing all three markers.

FIG. 22—Atlas of the human colon muscularis propria reveals 11 neuron subsets with roles in immunity and disease. (A) Census of the human muscularis propria. t-SNE of 134,835 RAISIN RNA-seq profiles from the muscularis propria of cancer-proximal macroscopically normal colon resections from 10 human donors, colored by cell type, annotated post hoc. (B) Enteric neuron census. t-SNE of 831 RAISIN RNA-seq profiles from enteric neurons, colored by subset, annotated post hoc. (C) Correspondence of human and mouse enteric neurons. Percent (dot size and color) of neurons from each human subset (rows) that matched each mouse neuron subset (column) according to the classifier (SOM). (D) Transcriptional signatures conserved between mouse and human neuron subsets. The fraction of expressing cells (dot size) and mean expression level in expressing cells (dot color) of selected genes (columns) identified as conserved for each neuron class (rows) between mouse (top) and human (bottom); full list available in table 23. (E-G) Characterization of ICCs in the colon (E) ICC gene signature. Fraction of expressing cells (dot size) and mean expression level in expressing cells (dot color) of selected ICC marker genes (columns) across human cell subsets (rows). (F) ICCs and not myocytes express receptors for nitric oxide. Distribution of expression levels (x axis, log₂(TP10K+1)) of acetylcholine (left) and nitric oxide (right) receptors across cell subsets (y axis). (G) In situ expression of key ICC markers in the human colon. (H) Proposed peristaltic circuitry. (I-J) Inferred cell-cell interactions networks for human cells in the mucosa and muscularis propria. (I) Statistically significant interactions. Nodes: cell subsets, annotated by type (color) and colon location (bold: muscularis). Edges connect pairs of cell subsets with a significant excess of cognate receptor-ligand pairs expressed (p<0.05) relative to a null model (SOM). (J) Select receptor-ligand interactions between neurons and adipocytes, fibroblasts, and immune cell subsets. (K,L) Representative in situ validations of IL-7 expression in NOS1+ neurons (K) and IL-12 expression in CHAT+ neurons (L).

FIG. 23—Human enteric neurons express disease risk genes for primary enteroneuropathies, IBD, and CNS disorders with concomitant gut dysmotility. Mean expression (scaled log₂(TP10K+1)) across cell subsets (rows) of putative risk genes (columns) implicated by GWAS for Hirschsprung's disease (HRSC), inflammatory bowel disease (IBD), autism spectrum disorders (ASD), and Parkinson's disease (PD) (SOM), which were identified as cell-specific in either (A) the colon mucosa, or (B) the colon muscularis propria.

FIG. 24—Mouse models for snRNA-seq optimization. (A-C) Labeling of nuclei in the mouse colon using different Cre-driver lines and conditional nuclear sfGFP (INTACT allele) (A,B), or regulatory region driving expression of nuclear mCherry (C). Representative images show cross-section of mouse colon with muscularis propria (bottom) and mucosa (top) (left). FACS plots (right) show enriched populations. (D) snRNA-seq of GFP⁺ nuclei from Sox10-Cre; INTACT animals. Fraction (y axis) of identified cell-types (x axis) in samples obtained from the brain (grey) and colon (black) using two previously published snRNA-seq methods (16, 17).

FIG. 25—Buffer optimization for snRNA-seq. (A) Decision tree for selection of best buffers. (B) RAISIN RNA-seq has optimal combination of ENS proportions and neuron quality scores. ENS signature score (y axis, mean and standard error of the mean (SEM); log₂(TP10K+1); SOM) and number of detected genes per nucleus (x axis, mean and SEM) for each of 36 total conditions. Dot size: percent neurons captured. Select nuclei extractions are marked in color (legend). (C-E) Quality scores across all tested parameters. Quality metrics (columns, x axes) for (C) a range of concentrations (y axes) across detergents, (D) mechanical extraction procedures, and (E) buffers.

FIG. 26—Extracted nuclei across different protocols. Representative phase contrast images of nuclei isolated using extractions with different detergents or extraction kits (grey, SOM) and buffers (blue), with varying detergent concentrations and additives (marked on image). All extractions were performed with the ‘chop’ method (SOM) unless otherwise indicated.

FIG. 27—Reproducibility and validations for the mouse ENS atlas. (A, B) Reproducible cell subset distributions across transgenic mouse lines and individual mice. t-SNE of RAISIN RNA-seq profiles of 2,447 neurons (A) and 2,734 glia (B) colored by cell subset (left), mouse model (middle), or donor mouse (right). (C) Neuron composition in colon. Percent of all cells in the colon that are neurons (y axis) as estimated by FACS (transgene expressing nuclei vs. unlabeled nuclei) and post-hoc adjustment using RAISIN RNA-seq data. (D) Chat⁺Nos1⁺ neurons. Representative images of Chat and Nos1 expression in neurons. (E) Nog⁺Grp⁺ neurons. Representative images of neurons that co-express Nog and Grp, showing they are not derived from the Sox10-Cre lineage (GFP).

FIG. 28—Representative in situ validations confirming the co-expression of marker genes for excitatory motor and sensory neurons. Grey-scale in situ validation showing co-expression of DAPI (blue) along with either (A) Piezo1 (green), Chat (red) and Tubb3 (white); inset: Piezo1⁺Chat⁺Tubb3⁺ PEMN; (B) Htr4 (green), Chat (red), and Tubb3 (white); inset: Htr4⁺Chat⁺Tubb3⁺ PEMN; (C) Htr4 (green), both forms of CGRP (red), and Tubb3 (white); top inset: Calca⁺Nos1⁺Tubb3⁺ PSN; bottom inset: Calcb⁺Nos1⁺Tubb3⁺ PSN; (D) Cck (green), Piezo2 (red), and Tubb3 (white); yellow inset: Cck⁺Piezo2⁺Tubb3⁺ PSN in muscularis propria; red inset: Cck⁺Piezo2⁺Tubb3⁺ PSN in lamina propria; or (E) Calcb (green), Chat (red), and Sst (white); inset: Calcb⁺Chat⁺Sst⁺ PSN.

FIG. 29—Expression profiles reveal key functions of mouse enteric neuron subsets. Fraction of expressing cells (dot size) and the mean levels in expressing (non-zero) cells (dot color) of select markers. (A) Major neurotransmitters and neuropeptides (left) and other genes (right) (columns), across neuron subsets (rows). (B) unique markers (columns) across neuron subsets (rows).

FIG. 30—Reproducible cell subset distributions across ten human donors. (A-F) Shared and donor-specific cell subsets in the human cell census. t-SNE of 134,835 RAISIN RNA-seq profiles (A,D), 831 neurons (B,E), or 6,878 glia from cancer-proximal colon resections collected from ten human donors, colored by cell subset (A-C) or patient identifier (D-F). Removal of oxidative phosphorylation (OXPHOS) signal in human neurons improved clustering by cell subset rather than cell state. t-SNE of human enteric neurons after removal of PC1 (G, identical to C) and before removal of PC1 (H-J) colored by cell subset, PC1 score (I), or OXPHOS expression score (J).

FIG. 31—Expression profiles reveal key functions of human enteric neuron subsets. Fraction of expressing cells (dot size) and the mean expression levels in expressing (non-zero) cells (dot color) of (A) major neurotransmitters and neuropeptides and (B) other genes (columns) across human neuron subsets (rows). Due to low levels of CHAT expression, Applicants used the acetylcholine transporter, SLC5A7, as a marker of cholinergic neurons.

FIG. 32—Human enteric neurons express disease risk genes for autism, Parkinson's disease, schizophrenia, and IBD. Mean expression (scaled log₂(TP10K+1)) across cell subsets (rows) of putative risk genes (columns) implicated by GWAS for autism, Parkinson's disease, schizophrenia, and IBD.

FIG. 33—Examples of multiple tissues and multiple individuals for analysis by single-cell genomics.

FIG. 34—Single nuclei RNA-seq analysis pipeline.

FIG. 35—Violin plots showing the number of genes detected per nuclei from two preparations of nuclei counting reads mapping to exons only or exons and introns.

FIG. 36—Graph showing the number of nuclei passing quality control from two preparations of nuclei counting reads mapping to exons only or exons and introns.

FIG. 37—Violin plots showing the number of genes detected per nuclei for nuclei subsets identified. The data was filtered using thresholds for single cell RNA-seq.

FIG. 38—Violin plots showing the number of genes detected per nuclei for nuclei subsets identified. The data was filtered using thresholds for single cell RNA-seq. Plot showing expression of TRAC in the nuclei subsets.

FIG. 39—Illustration of applying filters to remove data obtained from droplets containing a barcoded bead and doublets (two cells).

FIG. 40—Illustration of applying filters to remove data obtained from droplets containing ambient RNA.

FIG. 41—Example of clustering lung cell subsets from a tissue sample.

FIG. 42—Violin plots showing the number of genes detected per nuclei for four preparations from the same individual tissue.

FIG. 43—Violin plots showing the number of genes detected per nuclei for tissue samples from three individuals using the same nuclei preparation.

FIG. 44—Violin plots showing the proportion of reads mapping to mitochondrial genes from nuclei isolated from lung and heart tissues.

FIG. 45—tSNE plots combining single nuclei RNA-seq preparations from 12 samples. Left panel shows clusters identified. Right panel shows cells from each individual. Illustrates tSNE clusters cells by individuals without using batch correction.

FIG. 46—tSNE plots combining single nuclei RNA-seq preparations from 12 samples. Left panel shows clusters identified. Right panel shows cells from each individual. Illustrates tSNE clusters cells by cell type when using batch correction (see, e.g., LIGER: Josh Welch, Evan Macosko (BRAIN BICCN project), bioRxiv).

FIG. 47—tSNE plots for each sample after combining single nuclei RNA-seq preparations from the 12 samples. Each preparation shows similar clusters.

FIG. 48—Heat map showing differential gene expression between the nuclei subsets.

FIG. 49—tSNE of the single nuclei RNA-seq from the 12 lung samples showing clustering of the major subsets of parenchymal, stromal, and immune cells in lung tissue.

FIG. 50—tSNE of the Genotype-Tissue Expression (GTEx) project tissues after using improved single nuclei RNA-seq methods.

FIG. 51—Schematic showing detection of quantitative trait loci (QTLs) using the improved single nuclei RNA-seq pipeline and multiple individuals.

FIG. 52—tSNE representing nuclei from three individuals that was pooled together (top). tSNE showing demultiplexing of the nuclei (bottom).

FIG. 53A-53L—scRNA-Seq toolbox for fresh tumor samples. (53A, 53B) Study Overview. (53A) sc/snRNA-Seq workflow, experimental and computational pipelines, and protocol selection criteria. (53B) Tumor types in the study. Right column: recommended protocols for fresh (black/cells) or frozen (blue/nuclei) tumor samples. (53C) Flow chart for collection and processing of fresh tumor samples. (53D-53G) Comparison of three dissociation protocols applied to one NSCLC sample. (53D) Protocol performance varies across cell types. Top and middle: Distribution of number of reads/cell, number of UMI/cell, number of genes/cell, and fraction of mitochondrial reads (y axes) in each protocol (x axis) across the entire dataset, Bottom: Distribution of number of genes/cell (y axis) only in epithelial cells (left) or in B cells (right). (53E) Protocols vary in number of empty drops. UMAP embedding of single cell profiles (dots) for each protocol, colored by assignment as cell (grey) or empty drop (black). Horizontal bars: fraction of assigned cells (grey) and empty drops (black). (53F, 53G) Protocols vary in diversity of cell types captured. (53F) Top: UMAP embedding of single cell profiles (dots) from all three protocols, colored by assigned cell subset signature. Bottom: Proportion of cells in each subset in each of the three protocols, and in an analysis using CD45 depletion; n indicates the number of recovered cells passing QC. (53G) UMAP embedding as in (53F) colored by protocol. (53H-53L) Protocol comparison across tumor types. (53H) Cell type composition. Proportion of cells assigned to each cell subset signature (color) for each sample. R: Resection; B: Biopsy; A: Ascites; BD: Blood draw; O-PDX: Orthotopic patient-derived xenograft. (53I-53L) QC metrics. The median number of UMIs/cell, median number of genes/cell, median fraction of gene expression/cell from mitochondrial genes, and fraction of empty drops (x axes) for each sample in (53H) (y axis).

FIG. 54A-54J—snRNA-Seq toolbox for frozen tumor samples. (54A) Flow chart for collection and processing of frozen tumor samples. (54B-54D) Comparison of four nucleus isolation protocols in one neuroblastoma sample. (54B) Variation in protocol performance. Distribution of number of UMI/nucleus, number of genes/nucleus, and fraction of mitochondrial reads (y axes) in each protocol (x axis) across all nuclei in the dataset. (54C, 54D) Protocols vary in diversity of cell types captured. (54C) Top: UMAP embedding of single nucleus profiles (dots) from all four protocols, colored by assigned cell subset signature. Bottom: Proportion of cells from each subset in each of the four protocols. (54D) UMAP embedding as in (54C) colored by protocol. (54E-54H) Protocol comparison across tumor types. (54E) Cell-type composition. Proportion of cells assigned with each cell subset signature (color) for each sample. R: Resection; B: Biopsy; A: Ascites; BD: Blood draw; O-PDX: Orthotopic patient-derived xenograft. (54F-54H) QC metrics. Median number of UMI/nucleus, median number of genes/nucleus, and median fraction of gene expression/nucleus from mitochondrial genes for each sample in (54E). (54I-54J) scRNA-seq and snRNA-seq comparison in neuroblastoma. (54I) Compositional differences between scRNA-Seq and snRNA-Seq of the same sample. UMAP embedding of scRNA-seq and snRNA-Seq profiles of the same sample combined by CCA (Butler et al. Nature biotechnology 36:411-420 (2018)). (Methods) showing profiles (dots) from either scRNA-seq (left) or snRNA-Seq (right), colored by assigned cell type signatures. Bottom: Proportion of cells in each subset in the two protocols. (54J) Agreement in scRNA-seq and snRNA-seq intrinsic profiles. UMAP embedding as in (54I) showing both scRNA-seq and snRNA-Seq profiles, colored by assigned cell type signatures (top, colored as in (54I)) or by protocol (bottom).

FIG. 55—Overview of processed samples. Samples processed in this study are listed by tumor type (rows), along with their ID, tissue source (fresh or frozen, and OCT embedding), processing protocols tested, the recommended protocol, and the Figure showing the sample's analysis.

FIG. 56A-56O—ScRNA-Seq protocol comparison for one NSCLC sample. (45A) Sample processing and QC overview. For each protocol, shown are the number of cells passing QC, and the number of sequencing reads and sequencing saturation across all cells. The remaining metrics are reported for those cells passing QC: the median number of reads per cell, median number of UMIs per cell, median number of genes per cell, median fraction of UMIs mapping to mitochondrial genes, median fraction of duplicated UMIs per cell, fraction of cell barcodes called as empty droplets, and fraction of cell barcodes called as doublets. (56B) Read mapping QCs. The percent of bases in the sequencing reads (y axis) mapping to the genome, transcriptome, and intergenic regions (x axis) across the three protocols (colored bars). (56C-56D) Overall and cell types specific QCs. Distribution of the number of reads per cell, number of UMIs per cell, number of genes per cell, fraction of UMIs mapping to mitochondrial genes in each cell, and fraction of duplicated UMIs per cell (y axes) in each of the three protocols (x axis), for all cells passing QC (56C) and for cells passing QC from each cell type (56D, rows; if a protocol has no cells of that type, it is not shown). (56E, 56F) Relation of empty droplets and doublets to cell types. UMAP embedding of single cell (grey), “empty droplet” (red, top), and doublet (red, bottom) profiles for each protocol. (56G-56I) Cell type assignment. UMAP embedding of single cell profiles from each protocol colored by assigned cell type signature. (56J-56L) Inferred CNA profiles. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell. (56M-56O) Ambient RNA estimates. SoupX (Young et al. BioRxiv 303727 (2018)). estimates of the fraction of RNA in each cell type derived from ambient RNA contamination (y axis), with cell types ordered by their mean number of UMIs/cell (x axis). Red line: global average of contamination fraction; Green line: LOWESS smoothed estimate of the contamination fraction within each cell type, along with the associated confidence interval.

FIG. 57A-57H—ScRNA-Seq protocol comparison for NSCLC following read down-sampling. Shown are analyses for NSCLC14 (as in FIG. 56), but after the total number of sequencing reads within each sample was down-sampled to match the protocol with the fewest total sequencing reads. (57A) Sample processing and QC overview. For each protocol, shown are the number of cells passing QC. The remaining metrics are reported for those cells passing QC: median number of UMIs per cell, median number of genes per cell, median fraction of UMIs mapping to mitochondrial genes in each cell, fraction of cell barcodes called as empty droplets, and fraction of cell barcodes called as doublets. (57B, 57C) Overall and cell types specific QCs. Distribution of the number of UMIs per cell, number of genes per cell, and fraction of gene expression per cell from mitochondrial genes (y axes) in each of the three protocols (x axis), for all cells passing QC (57B) and for cells from each cell type (57C, rows; if a protocol has no cells of that type, it is not shown). (57D, 57E) Relation of empty droplets and doublets to cell types. UMAP embedding and fraction (horizontal bar) of single cell (grey), “empty droplet” (red, left), and doublet (red, right) profiles for each protocol (57F-57H) Cell type assignment. UMAP embedding of single cell profiles from each protocol colored by assigned cell type signature.

FIG. 58A-58I—Depletion protocol enriches for malignant cells in freshly processed NSCLC. Cells were processed using the PDEC protocol or the PDEC protocol combined with depletion of CD45⁺ cells. (58A) Sample processing and QC overview. For each protocol, shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (58B) Read mapping QCs. The percent of bases in the sequencing reads (y axis) mapping to the genome, transcriptome, and intergenic regions (x axis) in each of the two protocols (colored bars). (58C) Overall QCs. Distribution of the number of reads per cell, number of UMIs per cell, number of genes per cell, and fraction of UMIs mapping to mitochondrial genes in each cell (y axes) in each of the three protocols (x axis) for all cells passing QC. (58D, 58E) Relation of empty droplets and doublets to cell types. UMAP embedding and fraction (horizontal bar) of single cell (grey), “empty droplet” (red, left) and doublet (red, right) profiles for each protocol. (58F-58G) Cell type assignment. UMAP embedding of single cell profiles from each protocol colored by assigned cell type signature. (58H-58I) Inferred CNA profiles for cells from each protocol. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 59A-59I—Application of CD45⁺ cell depletion protocol for processing ascites from ovarian cancer. (59A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (59B) Read mapping QCs. The percent of bases in the sequencing reads (y axis) mapping to the genome, transcriptome, and intergenic regions (x axis). (59C) Overall QCs. Distribution of the number of reads per cell, number of UMIs per cell, number of genes per cell, and fraction of UMIs mapping to mitochondrial genes in each cell (y axes) for all cells passing QC. (59D, 59E) Relation of empty droplets and doublets to cell types. UMAP embedding and fraction (horizontal bar) of single cell (grey), “empty droplet” (red, left) and doublet (red, right) profiles. (59F) Cell type assignment. UMAP embedding of single cell profiles colored by assigned cell type signature. (59G, 59H) Flow-cytometry comparison of single cells isolated (59G) without or (59H) with depletion of CD45⁺ cells. Cells were gated by FSC and SSC (first column), doublets removed using FSC-A and FSC-H (second column), live cells identified using 7AAD (third column), and the distribution of immune and non-immune cells quantified using a CD45 antibody (fourth column). (59I) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 60A-60G—Protocol for lymph node resection of metastatic breast cancer. (60A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (60B) Read mapping QCs. The percent of bases in the sequencing reads (y axis) mapping to the genome, transcriptome, and intergenic regions (x axis). (60C) Overall QCs. Distribution of the number of reads per cell, number of UMIs per cell, number of genes per cell, and fraction of UMIs mapping to mitochondrial genes in each cell (y axes) for all cells passing QC. (60D, 60E) Relation of empty droplets and doublets to cell types. UMAP embedding and fraction (horizontal bar) of single cell (grey), “empty droplet” (red, left) and doublet (red, right) profiles. (60F) Cell type assignment. UMAP embedding of single cell profiles colored by assigned cell type signature. (60G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 61A-61G—Protocol for lymph node biopsy of metastatic breast cancer. (61A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of fraction of UMIs mapping to mitochondrial genes in each cell, fraction of cell barcodes called as empty droplets, and fraction of cell barcodes called as doublets. (61B) Read mapping QCs. The percent of bases in the sequencing reads (y axis) mapping to the genome, transcriptome, and intergenic regions (x axis). (61C) Overall QCs. Distribution of the number of reads per cell, number of UMIs per cell, number of genes per cell, and fraction of UMIs mapping to mitochondrial genes in each cell (y axes) for all cells passing QC. (61D, 61E) Relation of empty droplets and doublets to cell types. UMAP embedding and fraction (horizontal bar) of single cell (grey), “empty droplet” (red, left) and doublet (red, right) profiles. (61F) Cell type assignment. UMAP embedding of single cell profiles colored by assigned cell type signature. (61G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 62A-62G—Protocol for liver biopsy of metastatic breast cancer. (62A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (62B) Read mapping QCs. The percent of bases in the sequencing reads (y axis) mapping to the genome, transcriptome, and intergenic regions (x axis). (62C) Overall QCs. Distribution of the number of reads per cell, number of UMIs per cell, number of genes per cell, and fraction of UMIs mapping to mitochondrial genes in each cell (y axes) for all cells passing QC. (62D, 62E) Relation of empty droplets and doublets to cell types. UMAP embedding and fraction (horizontal bar) of single cell (grey), “empty droplet” (red, left) and doublet (red, right) profiles. (62F) Cell type assignment. UMAP embedding of single cell profiles colored by assigned cell type signature. (62G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 63A-63G—Protocol for liver biopsy of metastatic breast cancer. (63A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (63B) Read mapping QCs. The percent of bases in the sequencing reads (y axis) mapping to the transcriptome and intergenic regions (x axis). (63C) Overall QCs. Distribution of the number of reads per cell, number of UMIs per cell, number of genes per cell, and fraction of UMIs mapping to mitochondrial genes in each cell (y axes) for all cells passing QC. (63D, 63E) Relation of empty droplets and doublets to cell types. UMAP embedding and fraction (horizontal bar) of single cell (grey), “empty droplet” (red, left) and doublet (red, right) profiles. (63F) Cell type assignment. UMAP embedding of single cell profiles colored by assigned cell type signature. (63G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 64A-64G—Protocol for pre-treatment biopsy of neuroblastoma. (64A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (64B) Read mapping QCs. The percent of bases in the sequencing reads (y axis) mapping to the genome, transcriptome, and intergenic regions (x axis). (64C) Overall QCs. Distribution of the number of reads per cell, number of UMIs per cell, number of genes per cell, and fraction of UMIs mapping to mitochondrial genes in each cell (y axes) for all cells passing QC. (64D, 64E) Relation of empty droplets and doublets to cell types. UMAP embedding and fraction (horizontal bar) of single cell (grey), “empty droplet” (red, left) and doublet (red, right) profiles. (64F) Cell type assignment. UMAP embedding of single cell profiles colored by assigned cell type signature. (64G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 65A-65G—Protocol for post-treatment resection of neuroblastoma. (65A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (65B) Read mapping QCs. The percent of bases in the sequencing reads (y axis) mapping to the genome, transcriptome, and intergenic regions (x axis). (65C) Overall QCs. Distribution of the number of reads per cell, number of UMIs per cell, number of genes per cell, and fraction of UMIs mapping to mitochondrial genes in each cell (y axes) for all cells passing QC. (65D, 65E) Relation of empty droplets and doublets to cell types. UMAP embedding and fraction (horizontal bar) of single cell (grey), “empty droplet” (red, left) and doublet (red, right) profiles. (65F) Cell type assignment. UMAP embedding of single cell profiles colored by assigned cell type signature. (65G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 66A-66F—Protocol for O-PDX of neuroblastoma. (66A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (66B) Read mapping QCs. The percent of bases in the sequencing reads (y axis) mapping to the genome, transcriptome, and intergenic regions (x axis). (66C) Overall QCs. Distribution of the number of reads per cell, number of UMIs per cell, number of genes per cell, and fraction of UMIs mapping to mitochondrial genes in each cell (y axes) for all cells passing QC. (66D, 66E) Relation of empty droplets and doublets to cell types. UMAP embedding and fraction (horizontal bar) of single cell (grey), “empty droplet” (red, left) and doublet (red, right) profiles. (66F) Cell type assignment. UMAP embedding of single cell profiles colored by assigned cell type signature.

FIG. 67A-67G—Protocol for resection of neuroblastoma. (67A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (67B) Read mapping QCs. The percent of bases in the sequencing reads (y axis) mapping to the genome, transcriptome, and intergenic regions (x axis). (67C) Overall QCs. Distribution of the number of reads per cell, number of UMIs per cell, number of genes per cell, and fraction of UMIs mapping to mitochondrial genes in each cell (y axes) for all cells passing QC. (67D, 67E) Relation of empty droplets and doublets to cell types. UMAP embedding and fraction (horizontal bar) of single cell (grey), “empty droplet” (red, left) and doublet (red, right) profiles. (67F) Cell type assignment. UMAP embedding of single cell profiles colored by assigned cell type signature. (67G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 68A-68G—Protocol for resection of glioma. (68A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (68B) Read mapping QCs. The percent of bases in the sequencing reads (y axis) mapping to the genome, transcriptome, and intergenic regions (x axis). (68C) Overall QCs. Distribution of the number of reads per cell, number of UMIs per cell, number of genes per cell, and fraction of UMIs mapping to mitochondrial genes in each cell (y axes) for all cells passing QC. (68D, 68E) Relation of empty droplets and doublets to cell types. UMAP embedding and fraction (horizontal bar) of single cell (grey), “empty droplet” (red, left) and doublet (red, right) profiles. (68F) Cell type assignment. UMAP embedding of single cell profiles colored by assigned cell type signature. (68G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 69A-69G—Protocol for resection of ovarian cancer. (69A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (69B) Read mapping QCs. The percent of bases in the sequencing reads (y axis) mapping to the genome, transcriptome, and intergenic regions (x axis). (69C) Overall QCs. Distribution of the number of reads per cell, number of UMIs per cell, number of genes per cell, and fraction of UMIs mapping to mitochondrial genes in each cell (y axes) for all cells passing QC. (69D, 69E) Relation of empty droplets and doublets to cell types. UMAP embedding and fraction (horizontal bar) of single cell (grey), “empty droplet” (red, left) and doublet (red, right) profiles. (69F) Cell type assignment. UMAP embedding of single cell profiles colored by assigned cell type signature. (69G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 70A-70G—Protocol for cryopreserved sample of CLL. (70A) Sample processing and QC overview. Shown are the number of cells passing QC, and the number of sequencing reads and sequencing saturation across all cells. The remaining metrics are reported for those cells passing QC: median number of reads per cell, median number of UMIs per cell, median number of genes per cell, median fraction of UMIs mapping to mitochondrial genes in each cell, fraction of cell barcodes called as empty droplets, and fraction of cell barcodes called as doublets. (70B) Read mapping QCs. The percent of bases in the sequencing reads (y axis) mapping to the genome, transcriptome, and intergenic regions (x axis). (70C) Overall QCs. Distribution of the number of reads per cell, number of UMIs per cell, number of genes per cell, and fraction of UMIs mapping to mitochondrial genes in each cell (y axes) for all cells passing QC. (70D, 70E) Relation of empty droplets and doublets to cell types. UMAP embedding and fraction (horizontal bar) of single cell (grey), “empty droplet” (red, left) and doublet (red, right) profiles. (70F) Cell type assignment. UMAP embedding of single cell profiles colored by assigned cell type signature. (70G) Inferred CNA profiles for cells. Chromosomal amplification (red) and deletion (blue) inferred in each chromosomal position (columns) across the single cells (rows). Top: reference cells not expected to contain CNA in this cancer type. Bottom: cells tested for CNA relative to the reference cells. Color bar: assigned cell type signature for each cell.

FIG. 71A-71M—SnRNA-Seq protocol comparison for one neuroblastoma sample. (71A) Sample processing and QC overview. For each protocol, shown are the number of nuclei passing QC, and the number of sequencing reads and sequencing saturation across all nuclei. The remaining metrics are reported for those nuclei passing QC: the median number of reads per nucleus, median number of UMIs per nucleus, median number of genes per nucleus, median fraction of UMIs mapping to mitochondrial genes in each nucleus, median fraction of duplicated UMIs per nucleus, and fraction of nucleus barcodes called as doublets. (71B) Read mapping QCs. The percent of bases in the sequencing reads (y axis) mapping to the genome, transcriptome, and intergenic regions (x axis) across the four protocols (colored bars). (71C-71D) Overall and cell types specific QCs. Distribution of the number of reads per nucleus, number of UMIs per nucleus, number of genes per nucleus, fraction of UMIs mapping to mitochondrial genes in each nucleus, and fraction of duplicated UMIs per nucleus (y axes) in each of the four protocols (x axis), for all nuclei passing QC (71C) and for nuclei from each cell type (71D, rows; if a protocol has no cells of that type, it is not shown). (71E) Relation of doublets to cell types. UMAP embedding and fraction (horizontal bar) of single nucleus (grey) and doublet (red) profiles for each protocol. (71F-71I) Cell type assignment. UMAP embedding of single nucleus profiles from each protocol colored by assigned cell type signature. (71J-71M) Inferred CNA profiles. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 72A-72H—SnRNA-Seq protocol comparison for neuroblastoma following read down-sampling. Shown are analyses for NB HTAPP-244-SMP-451 (as in FIG. 71), but after the total number of sequencing reads within each sample was down-sampled to match the protocol with the fewest total sequencing reads. (72A) Sample processing and QC overview. For each protocol, shown are the number of nuclei passing QC. The remaining metrics are reported for those nuclei passing QC: median number of UMIs per nucleus, median number of genes per nucleus, median fraction of UMIs mapping to mitochondrial genes in each nucleus, and fraction of nucleus barcodes called as doublets. (72B, 72C) Overall and cell types specific QCs. Distribution of the number of UMIs per nucleus, number of genes per nucleus, and fraction of UMIs mapping to mitochondrial genes in each nucleus (y axes) in each of the four protocols (x axis), for all nuclei passing QC (72B) and for nuclei from each cell type (72C, rows; if a protocol has no cells of that type, it is not shown). (72D) Relation of doublets to cell types. UMAP embedding and fraction (horizontal bar) of single nucleus (grey) and doublet (red) profiles for each protocol. (72E-72H) Cell type assignment. UMAP embedding of single nucleus profiles from each protocol colored by assigned cell type signature.

FIG. 73A-73H—Protocol comparison for resection of a breast cancer metastasis from the brain. (73A) Sample processing and QC overview. For each protocol, shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (73B) Read mapping QCs. The percent of bases in the sequencing reads (y axis) mapping to the genome, transcriptome, and intergenic regions (x axis). (73C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (73D) Relation of doublets to cell types. UMAP embedding and fraction (horizontal bar) of single nucleus (grey) and doublet (red) profiles for each protocol. (73E-73F) Cell type assignment. UMAP embedding of single nucleus profiles from each protocol colored by assigned cell type signature. (73G-73H) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 74A-74H—Protocol comparison for resection of metastatic breast cancer from the brain. (74A) Sample processing and QC overview. For each protocol, shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (74B) Read mapping QCs. The percent of bases in the sequencing reads (y axis) mapping to the genome, transcriptome, and intergenic regions (x axis). (74C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (74D) Relation of doublets to cell types. UMAP embedding and fraction (horizontal bar) of single nucleus (grey) and doublet (red) profiles for each protocol. (74E-74F) Cell type assignment. UMAP embedding of single nucleus profiles from each protocol colored by assigned cell type signature. (74G-74H) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 75A-75H—Protocol comparison for biopsy of metastatic breast cancer from the liver. (75A) Sample processing and QC overview. For each protocol, shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (75B) Read mapping QCs. The percent of bases in the sequencing reads (y axis) mapping to the genome, transcriptome, and intergenic regions (x axis). (75C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (75D) Relation of doublets to cell types. UMAP embedding and fraction (horizontal bar) of single nucleus (grey) and doublet (red) profiles for each protocol. (75E-75F) Cell type assignment. UMAP embedding of single nucleus profiles from each protocol colored by assigned cell type signature. (75G-75H) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 76A-76J—Protocol comparison for resection of ovarian cancer. (76A) Sample processing and QC overview. For each protocol, shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (76B) Read mapping QCs. The percent of bases in the sequencing reads (y axis) mapping to the genome, transcriptome, and intergenic regions (x axis). (76C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (76D) Relation of doublets to cell types. UMAP embedding and fraction (horizontal bar) of single nucleus (grey) and doublet (red) profiles for each protocol. (76E-76G) Cell type assignment. UMAP embedding of single nucleus profiles from each protocol colored by assigned cell type signature. (76H-76J) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 77A-77H—Protocol comparison for resection of sarcoma. (77A) Sample processing and QC overview. For each protocol, shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes, and fraction of nucleus barcodes called as doublets. (77B) Read mapping QCs. The percent of bases in the sequencing reads (y axis) mapping to the genome, transcriptome, and intergenic regions (x axis). (77C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (77D) Relation of doublets to cell types. UMAP embedding and fraction (horizontal bar) of single nucleus (grey) and doublet (red) profiles for each protocol. (77E-77F) Cell type assignment. UMAP embedding of single nucleus profiles from each protocol colored by assigned cell type signature. (77G-77H) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 78A-78F—Protocol for resection of glioma. (78A) Sample processing and QC overview. Shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (78B) Read mapping QCs. The percent of bases in the sequencing reads (y axis) mapping to the genome, transcriptome, and intergenic regions (x axis). (78C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (78D) Relation of doublets to cell types. UMAP embedding and fraction (horizontal bar) of single nucleus (grey) and doublet (red) profiles. (78E) Cell type assignment. UMAP embedding of single nucleus profiles colored by assigned cell type signature. (78F) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 79A-79E—Protocol for O-PDX of neuroblastoma. (79A) Sample processing and QC overview. Shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (79B) Read mapping QCs. The percent of bases in the sequencing reads (y axis) mapping to the genome, transcriptome, and intergenic regions (x axis). (79C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (79D) Relation of doublets to cell types. UMAP embedding and fraction (horizontal bar) of single nucleus (grey) and doublet (red) profiles. (79E) Cell type assignment. UMAP embedding of single nucleus profiles colored by assigned cell type signature.

FIG. 80A-80F—Protocol for resection of neuroblastoma. (80A) Sample processing and QC overview. Shown are the number of nuclei passing QC, and the number of sequencing reads and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (80B) Read mapping QCs. The percent of bases in the sequencing reads (y axis) mapping to the genome, transcriptome, and intergenic regions (x axis). (80C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (80D) Relation of doublets to cell types. UMAP embedding and fraction (horizontal bar) of single nucleus (grey) and doublet (red) profiles for each protocol. (80E) Cell type assignment. UMAP embedding of single nucleus profiles from each protocol colored by assigned cell type signature. (80F) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 81A-81F—Protocol for resection of sarcoma. (81A) Sample processing and QC overview. Shown are the number of nuclei passing QC, the number of sequencing reads, and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (81B) Read mapping QCs. The percent of bases in the sequencing reads (y axis) mapping to the genome, transcriptome, and intergenic regions (x axis). (81C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (81D) Relation of doublets to cell types. UMAP embedding and fraction (horizontal bar) of single nucleus (grey) and doublet (red) profiles. (81E) Cell type assignment. UMAP embedding of single nucleus profiles colored by assigned cell type signature. (81F) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 82A-82F—Protocol for resection of melanoma. (82A) Sample processing and QC overview. Shown are the number of nuclei passing QC, the number of sequencing reads, and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (82B) Read mapping QCs. The percent of bases in the sequencing reads (y axis) mapping to the genome, transcriptome, and intergenic regions (x axis). (82C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (82D) Relation of doublets to cell types. UMAP embedding and fraction (horizontal bar) of single nucleus (grey) and doublet (red) profiles. (82E) Cell type assignment. UMAP embedding of single nucleus profiles colored by assigned cell type signature. (82F) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 83A-83F—Protocol for resection of melanoma. (83A) Sample processing and QC overview. Shown are the number of nuclei passing QC, the number of sequencing reads, and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (83B) Read mapping QCs. The percent of bases in the sequencing reads (y axis) mapping to the genome, transcriptome, and intergenic regions (x axis). (83C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (83D) Relation of doublets to cell types. UMAP embedding and fraction (horizontal bar) of single nucleus (grey) and doublet (red) profiles. (83E) Cell type assignment. UMAP embedding of single nucleus profiles colored by assigned cell type signature. (83F) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 84A-84F—Protocol for cryopreserved sample of CLL. (84A) Sample processing and QC overview. Shown are the number of nuclei passing QC, the number of sequencing reads, and sequencing saturation across all nuclei. The remaining metrics are reported for those nuclei passing QC: median number of reads per nucleus, median number of UMIs per nucleus, median number of genes per nucleus, median fraction of UMIs mapping to mitochondrial genes in each nucleus, and fraction of nucleus barcodes called as doublets. (84B) Read mapping QCs. The percent of bases in the sequencing reads (y axis) mapping to the genome, transcriptome, and intergenic regions (x axis). (84C) Overall QCs. Distribution of the number of reads per nucleus, number of UMIs per nucleus, number of genes per nucleus, and fraction of UMIs mapping to mitochondrial genes in each nucleus (y axes) for all nuclei passing QC. (84D) Relation of doublets to cell types. UMAP embedding and fraction (horizontal bar) of single nucleus (grey) and doublet (red) profiles. (84E) Cell type assignment. UMAP embedding of single nucleus profiles colored by assigned cell type signature. (84F) Inferred CNA profiles for nuclei. Chromosomal amplification (red) and deletion (blue) inferred in each chromosomal position (columns) across the single nuclei (rows). Top: reference nuclei not expected to contain CNA in this cancer type. Bottom: nuclei tested for CNA relative to the reference nuclei. Color bar: assigned cell type signature for each nucleus.

FIG. 85A, 85B—Protocol comparison of V2 and V3 chemistry from 10× Genomics on a resection of sarcoma. (85A) Sample processing and QC overview. For each protocol, shown are the number of nuclei passing QC, after the total number of sequencing reads from the V3 protocol data was down-sampled to match the number of reads in the V2 data. The remaining metrics are reported for those nuclei passing QC: median number of UMIs per nucleus, median number of genes per nucleus, median fraction of UMIs mapping to mitochondrial genes in each nucleus, and fraction of nucleus barcodes called as doublets. (85B) Overall QCs. Distribution of number of UMIs per nucleus, number of genes per nucleus, and fraction of UMIs mapping to mitochondrial genes in each nucleus (y axes) for all nuclei passing QC.

FIG. 86A-86C—Comparison of scRNA-Seq and snRNA-Seq from a single blood draw sample of CLL (CLL1). (86A-86C) UMAP embedding of single cell and single nucleus profiles after batch correction by CCA (Methods) colored by either assigned cell type signature (86A; fractions in horizontal bar), cluster assignment (86B) or data type (c, cells or nuclei; horizontal bar: cluster assignment).

FIG. 87A-87C—Comparison of scRNA-Seq and snRNA-Seq from a single metastatic breast cancer sample (HTAPP-963-SMP-4741). (87A-87C) UMAP embedding of single cell and single nucleus profiles after batch correction by CCA (Methods) colored by either assigned cell type signature (87A; fractions in horizontal bar), cluster assignment (87B) or data type (87C, cells or nuclei; horizontal bar: cluster assignment).

FIG. 88A-88C—Comparison of scRNA-Seq and snRNA-Seq from a single neuroblastoma sample (HTAPP-656-SMP-3481). (88A-88C) UMAP embedding of single cell and single nucleus profiles after batch correction by CCA (Methods) colored by either assigned cell type signature (88A; fractions in horizontal bar), cluster assignment (88B) or data type (88C, cells or nuclei; horizontal bar: cluster assignment).

FIG. 89A-89C—Comparison of scRNA-Seq and snRNA-Seq from a single O-PDX neuroblastoma sample. (89A-89C) UMAP embedding of single cell and single nucleus profiles after batch correction by CCA (Methods) colored by either assigned cell type signature (89A; fractions in horizontal bar), cluster assignment (89B) or data type (89C, cells or nuclei; horizontal bar: cluster assignment).

FIG. 90—Validation of the Sox10-Cre driver. Triple-transgenic mice harboring Sox10-Cre; INTACT; conditional tdTomato alleles were used to evaluate concordance of genetically labeled cells and TUBB3 immunofluorescence.

FIG. 91A-91C—High quality neuron and glia transcriptomes. Mean expression levels (log 2(TP10K+1)) of hallmark genes (x axis) across cell subsets (y axis) for major cell classes (91A), neuron subsets (91B), or glia subsets (91C). Cell subsets were profiled using either Smart-Seq2 (SS2) or droplet-based methods.

FIG. 92A-92F—Detection of Tph2 expression in the brain, but not colon. (92A) Schematic of coronal brain section. Raphe nuclei contain serotonergic (Tph2+) neurons and served as a positive control. The pontine reticular nucleus does not contain Tph2-expressing neurons and served as a negative control. (92B, 92C) Representative images of smFISH for Tph2 in the mouse brain (92B) and colon (92C) of Sox10-Cre; INTACT (GFP) mice (n=2 animals; 12 colon sections). (92D, 92E) Representative images of smFISH for Tph2 in the mouse brain (92B) and colon (92C) of wild-type C57BL/6J mice. (n=2 animals; 12 colon sections). (92F) Analysis of bulk RNA-seq data from several tissues of C57BL/6 mice (Sollner et al. 2017). RNA expression of Tph1 and Tph2 from the brain, colon and small intestine. RNA expression independently analyzed in three mice per tissue is indicated 1-3.

FIG. 93—An overview of cloud-based analysis. The flow chart and table show that the pipeline for cloud based analysis after data processing is efficient and quick—it allows one analyze about a million cells within 2 hours as compared to runs that take days. It is also shareable and reproducible.

FIGS. 94A-94B—Fresh tissue test case for non-small cell lung carcinoma (NSCLC). (94A) Technical QCs for three different cell dissociation protocols. While the QCs look similar, each protocol results in a different proportion of cell types. (95B) Cell type diversity achieved from each protocol. NSCLC samples from all three cell dissociation protocols are embedded. Similar numbers of cells were recovered across protocols, but different cell type proportions.

FIG. 95—Cell type-specific QCs for three different dissociation protocols. The C4 protocol has the greatest number of genes detected per cell overall. The LE protocol has the greatest number of genes detected per cell in epithelial cells. The PDEC protocol has the greatest number of genes detected per cell in B cells.

FIG. 96—The fresh tumor toolbox was used successfully across six tumor types. Five types of fresh tumors were processed: non-small cell lung carcinoma (NSCLC), metastatic breast cancer (MBC), ovarian cancer, glioblastoma (GBM), and neuroblastoma, as well as a cryopreserved non-solid, chronic lymphocytic leukemia (CLL).

FIG. 97—QC assessment across all cells in a sample and per cell type for tumors processed in FIG. 96. QCs and cell proportions were measured for all of them. A recommended protocol was chosen for each tumor type.

FIG. 98—Workflow of single nucleus RNA-seq from frozen tissue.

FIG. 99—snRNA-seq toolbox for processing frozen tissue. The best approach was testing four different nucleus isolation buffers, three of which were very similar to each other apart from the detergent and the original buffer EZ.

FIG. 100—The frozen tumor toolbox was used successfully across 7 tumor types.

FIG. 101—snRNA -seq of pre-malignant breast ductal carcinoma in situ (DCIS). Analysis revealed pretty good QCs and Applicants were able to detect several cell types—including two clusters of epithelial cells, immune cells, endothelial cells, and fibroblasts.

FIG. 102—Detection of specific breast cancer markers.

FIG. 103—Optimization strategy for snRNA-seq of FFPE samples.

FIG. 104—Workflow for snRNA-seq of FFPE samples.

FIG. 105—Single-nucleus RNA-seq was tested on FFPE samples. Shown are (105A) human lung cancer and (105B) mouse brain tissue in FFPE block. The samples were prepared fresh and processed quickly.

FIG. 106—Summary of optimization steps for processing FFPE tissue. Two different library construction (LC) methods were used: SCRB-Seq and Smart-seq2.

FIG. 107—Optimization of methods for WTA and library construction (LC).

FIG. 108A-108B—QCs for SMART-Seq2 and SCRB-Seq. In 108B, Applicants used mineral oil for analysis of number of genes only.

FIG. 109—Correlation across treatment, library prep and number of nuclei. As expected, the correlation goes down with the numbers of nuclei tested—since mouse cortex is a complex tissue with many cell types. Correlation across preps 100>10>1.

FIG. 110—Profiling nuclei from mouse brain FFPE reveals expression of cortex genes. There were 65 single nuclei in total. No clear clusters were detected after accounting for batch/library type. Differential expression of known mouse cortex cell type markers was detected.

FIGS. 111A-111B—Nuclei profiled from mouse brain FFPE are predicted to map to mouse cortex cell types. The prediction accuracy was 0.69.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. provisional application 62/734,988, filed Sep. 21, 2018 and PCT/US2018/060860, filed Nov. 13, 2018.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide for methods of analyzing single cells from archived tissue samples or tissue samples that cannot be immediately processed (e.g., FFPE or frozen tissue). Embodiments disclosed herein also provide for methods of analyzing rare or difficult to isolate cells (e.g., neurons). Tissue processing directly for single cell or single nuclei genomics advantageously provides for the ability to analyze archival samples, longitudinal samples, samples that are shipped worldwide, samples from rare diseases, and/or samples that have well documented pathology.

It is an objective of the present invention to use single cell methods on FFPE tissue samples. Single nuclei or whole cells can be isolated from FFPE tissue samples for use in analyzing single cells in archived samples or samples that cannot be immediately processed. In certain embodiments, pre-malignant lesions or tissues from cancer patients are analyzed. In certain embodiments, the methods can be used to generate an atlas of pre-cancer and cancer tissues. Most tissues are small and preserved as FFPE and present many challenges. FFPE may damage the cell and nuclear membranes, damages the RNA and cross-links nucleotides and the FFPE protocol varies (e.g. fixation time, storage). Applicants have previously performed single nucleus RNA-seq from frozen tissue. Applicants provide methods of isolating whole cells and nuclei from FFPE tissues that can be used in single cell methods.

It is an objective of the present invention to use single cell methods on nuclei isolated from tissue samples containing rare or difficult to isolate cells. Embodiments disclosed herein provide for methods of isolating nuclei, including ribosomes or ribosomes and rough ER, from tissue samples for use in analyzing single cells, preferably, in frozen samples or samples that cannot be immediately processed. As the largest branch of the autonomic nervous system, the enteric nervous system (ENS) controls the entire gastrointestinal (GI) tract tract, but remains incompletely characterized. However, its sparsity and location within the structurally resilient GI wall has precluded the application of modern single cell genomics approaches. Here, Applicants developed RAISIN RNA-seq, which enables the capture of ribosome bound mRNA along with intact single nuclei, and use it to profile the adult mouse and human colon to generate a reference map of the ENS at a single cell level, profiling 2,447 mouse and 831 human enteric neurons This map reveals an extraordinary diversity of neuron subtypes across intestinal locations, ages, and the circadian rhythm, with conserved transcriptional programs between human and mouse. The methods provided for novel insight into ENS function that was not possible using previous methods. Applicants further highlight possible revisions to the current model of peristalsis and molecular mechanisms that may allow enteric neurons to orchestrate tissue homeostasis, including immune regulation and stem cell maintenance. Lastly, Applicants show that human enteric neurons specifically express risk genes for neuropathic, inflammatory, and extra-intestinal diseases with concomitant gut dysmotility.

It is another objective of the present invention to use novel therapeutic targets, diagnostic targets and methods of screening for modulating agents based on the characterization of the ENS described further herein. The study described herein provides a roadmap to understanding the ENS in health and disease. The GWAS disease risk genes are now shown to be expressed in neurons. Therefore, diseases can be treated by targeting the neurons specifically. Specific therapeutic targets include markers for each neuron, transcriptional core programs, or neurotransmitter and receptor pairs. The neurons are also shown to affect immune cells. Therefore, the diseases originally not connected to immunity can be treated with anti-immune therapy (e.g., targeting IL-7, IL-12, IL-15).

It is another objective of the present method to provide nuclei specific methods of analysis for single nuclei sequencing. Applicants show improved recovery of genes and cells by counting both exons and introns and using nuclei specific filtering and batch correction.

Methods of Recovering Nuclei or Whole Cells from FFPE Tissue

In certain embodiments the invention provides methods for recovering nuclei or whole cells from a formalin-fixed paraffin-embedded (FFPE) tissue comprising dissolving paraffin from a FFPE tissue sample in a solvent, preferably a solvent selected from the group consisting of xylene and mineral oil. The tissue may be dissolved at a temperature between 4 C to 90 C, preferably room temperature (20 to 25 C) for recovering whole cells and 90 C for recovering nuclei. The tissue may be rehydrated using a gradient of ethanol from 100% to 0% ethanol (EtOH). The rehydrated tissue may be transferred to a volume of a first buffer comprising a buffering agent, a detergent and an ionic strength between 100 mM and 200 mM. Optionally the first buffer comprises protease inhibitors or proteases and/or BSA. The tissue may then be chopped or dounce homogenized in the buffer and the debris may be removed by filtering and/or FACS sorting.

Tissue Samples

The tissue sample for use with the present invention may be obtained from the brain. The tissue sample may be obtained from the gut. In certain embodiments, brain and gut cells are difficult to analyze by single cell RNA sequencing due to cell morphology. In certain embodiments, single nuclei sequencing can overcome difficulty in analyzing rare cells in the gut and brain due to cell morphology. In certain embodiments, the present invention provides for genetic targeting of rare cells in a complex tissue.

In certain embodiments, the tissue sample may be obtained from the heart, lung, prostate, skeletal muscle, esophagus, skin, breast, prostate, pancreas, or colon.

In certain embodiments, the tissue sample is obtained from a subject suffering from a disease. Since samples may be frozen and analyzed by single nuclei sequencing, samples from many diseased patients may be analyzed at once. The samples do not need to be analyzed immediately after removal from a subject. Diseased samples may be compared to healthy samples and differentially genes may be detected. In certain embodiments, the disease is autism spectrum disorder. Other diseases may include, but are not limited to, cancer (e.g., brain cancer) and irritable bowel disease (IBD). In certain embodiments, the disease can be any disease described herein (see, e.g., Examples).

Previous methods (e.g., including commercial methods) for isolating nuclei contain lysis buffers incapable of preserving a portion of the outer nuclear envelope and ribosomes, outer nuclear envelope, rough endoplasmic reticulum (RER) with ribosomes, or outer nuclear envelope, RER, and mitochondria. Before the present invention it was not appreciated that gene expression of single cells may be improved by isolating nuclei that include a portion of the outer nuclear envelope, and/or attached ribosomes, and/or rough endoplasmic reticulum (RER). In certain embodiments, the ribosomes and/or RER is a site of RNA translation and includes fully spliced mRNA. Preserving a portion of the RER improves RNA recovery and single cell expression profiling.

In certain embodiments, single nuclei comprising ribosomes and/or RER are isolated using lysis buffers comprising detergent and salt. In certain embodiments, the ionic strength of the buffer is between 100 and 200 mM. As used herein the term “ionic strength” of a solution refers to the measure of electrolyte concentration and is calculated by:

μ=1/2Σcᵢzᵢ²

where c is the molarity of a particular ion and z is the charge on the ion.

In certain embodiments, the ionic strength of the lysis solution can be obtained with salts, such as, but not limited to NaCl, KCl, and (NH4)2SO4. For example, the buffer can comprise 100-200 mM NaCl or KCl (i.e., ionic strength 100-200 mM). In one embodiment, the salt comprises NaCl and the concentration is 146 mM.

In certain embodiments, the buffer comprises CaCl2. The CaCl2 may be about 1 mM. In certain embodiments, the buffer comprises MgCl2. The MgCl2 may be about 21 mM.

In certain embodiments, the buffer comprises a detergent concentration that preserves a portion of the outer nuclear envelope and/or ribosomes, and/or rough endoplasmic reticulum (RER). The detergent may be an ionic, zwitterionic or nonionic detergent. The detergent concentration may be a concentration that is sufficient to lyse cells, but not strong enough to fully dissociate the outer nuclear membrane and RER or detach ribosomes. In certain embodiments, the detergent is selected from the group consisting of NP40, CHAPS and Tween-29. Detergent concentrations may be selected based on the critical micelle concentration (CMC) for each detergent (Table 1). The concentration may be varied above and below the CMC. In certain embodiments, the detergent concentration in the lysis buffer of the present invention comprises about 0.2% NP40, about 0.49% CHAPS, or about 0.03% Tween-20. The critical micelle concentration (CMC) is defined as the concentration of surfactants above which micelles form and all additional surfactants added to the system go to micelles. Before reaching the CMC, the surface tension changes strongly with the concentration of the surfactant. After reaching the CMC, the surface tension remains relatively constant or changes with a lower slope.

The isolated nuclei comprising a preserved portion of the outer membrane and RER and/or ribosomes may be further analyzed by single nuclei sequencing, droplet single nuclei sequencing or Div-seq as described in international application number PCT/US2016/059239 published as WO/2017/164936. In certain embodiments, single nuclei are sorted into separate wells of a plate. In certain embodiments, single nuclei are sorted into individual droplets. The droplets may contain beads for barcoding the nucleic acids present in the single nuclei. The plates may include barcodes in each well. Thus, barcodes specific to the nuclei (i.e., cell) of origin may be used to determine gene expression in single cells.

TABLE 1

| | MW | | | gram per | % w/v |
|---|---|---|---|---|---|
| | (Da) | CMC | | 1 mL | CMC |
| Nonidet P-40/ | ~603 | 0.08 mM(sigma); | | 0.00048 | 0.048% |
| IGEPAL | | 0.05-0.3 mM (anatrace) | | | |
| CA-630 | | | | | |
| Tween-20 | 1228 | 0.049 | mM | 0.00006 | 0.006% |
| Digitonin | 70000 | <0.5 | mM | 0.035 | 3.5% |
| CHAPS | 614.9 | 8 to 10 | mM | 0.00492 | 0.49% |

Exemplary nuclei purification protocols may be used with a lysis buffer of the present invention (Table 2).

TABLE 2

| | | | | Detergent | | |
|---|---|---|---|---|---|---|
| | | Buffer | | concentration | Salt and | Additives and |
| Composition | Buffer | concentration | Detergent | (%) | concentration | concentration |
| 1 | Tris | 10 mM | NP40 | 0.2 | 146 mM NaCl, 1 mM | |
| | | | | | CaCl2, 21 mM MgCl2 | |
| 2 | Tris | 10 mM | CHAPS | 0.49 | 146 mM NaCl, 1 mM | |
| | | | | | CaCl2, 21 mM MgCl2 | |
| 3 | Tris | 10 mM | Tween-20 | 0.03 | 146 mM NaCl, 1 mM | |
| | | | | | CaCl2, 21 mM MgCl2 | |
| 4 | Tricine | 20 mM | NP40 | 0.2 | 146 mM NaCl, 1 mM | 0.15 mM |
| | | | | | CaCl2, 21 mM MgCl2 | spermine and |
| | | | | | | 0.5 mM |
| | | | | | | spermidine |

One of skill in the art will recognize that methods and systems of the invention are not limited to any particular type of sample or tissue type, and methods and systems of the invention may be used with any type of organic, inorganic, or biological molecule (see, e.g, US Patent Publication No. 20120122714). In particular embodiments the sample may include nucleic acid target molecules. Nucleic acid molecules may be synthetic or derived from naturally occurring sources. In one embodiment, nucleic acid molecules may be isolated from a biological sample containing a variety of other components, such as proteins, lipids and non-template nucleic acids. Nucleic acid target molecules may be obtained from any cellular material, obtained from an animal, plant, bacterium, fungus, or any other cellular organism. In certain embodiments, the nucleic acid target molecules may be obtained from a single cell. Biological samples for use in the present invention may include viral particles or preparations. Nucleic acid target molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid target molecules may also be isolated from cultured cells, such as a primary cell culture or a cell line. The cells or tissues from which target nucleic acids are obtained may be infected with a virus or other intracellular pathogen. A sample may also be total RNA extracted from a biological specimen, a cDNA library, viral, or genomic DNA. Tissues may be freshly dissected, frozen tissue, or fixed tissue. In specific embodiments, the tissues are frozen in clear tubes.

Nucleic acid obtained from biological samples typically may be fragmented to produce suitable fragments for analysis. Target nucleic acids may be fragmented or sheared to desired length, using a variety of mechanical, chemical and/or enzymatic methods. DNA may be randomly sheared via sonication, e.g. Covaris method, brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. In one embodiment, nucleic acid from a biological sample is fragmented by sonication. In another embodiment, nucleic acid is fragmented by a hydroshear instrument. Generally, individual nucleic acid target molecules may be from about 40 bases to about 40 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent may be up to an amount where the detergent remains soluble in the solution. In one embodiment, the concentration of the detergent is between 0.1% to about 2%. The detergent, particularly a mild one that is nondenaturing, may act to solubilize the sample. Detergents may be ionic or nonionic. Examples of nonionic detergents include triton, such as the Triton™ X series (Triton™ X-100 t-Oct-C6H4-(OCH2-CH2)xOH, x=9-10, Triton™ X-100R, Triton™ X-114 x=7-8), octyl glucoside, polyoxyethylene(9)dodecyl ether, digitonin, IGEPAL™ CA630 octylphenyl polyethylene glycol, n-octyl-beta-D-glucopyranoside (betaOG), n-dodecyl-beta, Tween™ 20 polyethylene glycol sorbitan monolaurate, Tween™ 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14E06), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulfonate. It is contemplated also that urea may be added with or without another detergent or surfactant.

In some embodiments, the paraffin from a FFPE tissue sample may be dissolved in any suitable solvent known in the art. Such solvents include, but are not necessarily limited to, xylene, toluene, mineral oil, and vegetable oil. In specific embodiments, the solvent is xylene. In specific embodiments, the solvent is mineral oil.

In some embodiments, the tissue may be dissolved at a temperature ranging from 4° C. to 90° C., such as at 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C.

In specific embodiments, the tissue may be dissolved at room temperature for the purpose of recovering whole cells, such as at a temperature ranging between 20° C. and 25° C.

In specific embodiments, the tissue may be dissolved at 90° C. for the purpose of recovering nuclei.

In specific embodiments, dissolving paraffin from a FFPE tissue sample comprises incubating at least one time in xylene, at room temperature (RT), for about 10 minutes each, wherein xylene is removed at each change.

In specific embodiments, the tissue may be washed at least two times with xylene for about 10 min each. The washes may be performed at room temperature (RT), 90 C, or at least one time at room temperature (RT) and at least one time at 90 C, wherein xylene is removed at each change.

In specific embodiments, dissolving paraffin from a FFPE tissue sample comprises incubating at least twice in about 5 ml xylene per 30-100 mg FFPE tissue sample, at room temperature, for about 10 minutes each, wherein xylene is removed at each change. As such, the tissue may be washed with xylene at 37 C for about 10 min.

The method may further comprise cutting the tissue into two or more pieces and washing at least one piece of the tissue with xylene at 37 C for about 10 min.

In some embodiments, dissolving paraffin from a FFPE tissue sample comprises incubating the sample at least three times in xylene, at room temperature, for about 10 minutes each, and wherein xylene is removed at each change.

The method may further comprise washing the tissue three additional times with xylene for about 10 min each, wherein the first wash is at room temperature and the second and third washes are at 90 C, and wherein xylene is removed at each change.

In some embodiments, after the step of dissolving paraffin from the tissue or rehydrating the tissue the method further comprises dividing the tissue, preferably in half.

The tissue may be rehydrated using a step gradient of ethanol in concentrations ranging from 100° C. to 0° C. ethanol (EtOH). The tissue may be incubated between 1 to 10 minutes at each step. For example, the step gradient may comprise incubating the tissue for about two minutes each in successive washes of 95% ethanol, 75% ethanol, and 50% ethanol, or any other suitable method known in the art. In some embodiments, after the tissue is rehydrated, the method may further comprise placing the tissue samples on ice or on a device capable of maintaining the tissue between 4 and 10 C, wherein all subsequent steps are performed at a temperature between 4 and 10 C.

Rehydrated tissue may be transferred to a volume of a first buffer comprising a buffering agent, a detergent and an ionic strength between 100 mM and 200 mM. Optionally the first buffer comprises protease inhibitors or proteases and/or BSA.

In some embodiments, the first buffer comprises a detergent selected from the group consisting of NP40, CHAPS and Tween-20. In some embodiments, the NP40 concentration may be about 0.2%. In some embodiments, the Tween-20 concentration may be about 0.03%. In some embodiments, the CHAPS concentration may be about 0.49%. In some embodiments, the first buffer may be selected from the group consisting of CST, TST, NST and NSTnPo.

The tissue may be chopped or dounce homogenized in the buffer. Non-limiting examples of chopping include cutting with scissors, chopping with a scalpel or any blade known in the art. Chopping may be manual. Chopping may use any device known in the art capable of chopping. Any method for dounce homogenizing known in the art may be used. An exemplary method for dounce homogenization is described in the examples.

In some embodiments, after the step of chopping or dounce homogenizing the method may further comprise centrifuging. Preferably, the sample is centrifuged at about 500 g for about 5 min, and the sample is then resuspended in a second buffer comprising a buffering agent and an ionic strength between 100 mM and 200 mM. Optionally the second buffer comprises protease inhibitors. In some embodiments, the second buffer is ST, optionally comprising protease inhibitors.

Debris may be removed by methods including, but not necessarily limited to, filtering and/or FACS sorting. In some embodiments, the sample is filtered through a 40 uM filter. In some embodiments, the sample is filtered through a 30 uM filter. In some embodiments, the method may further comprise washing the filtered sample in the first buffer.

In some embodiments, after the step of chopping or dounce homogenizing the method may further comprise adding an additional 2 volumes of the first buffer (3 volumes total) and filtering the sample through a 40 uM filter.

In some embodiments, the method may further comprise adding an additional three volumes of the first buffer (6 volumes total). The sample is then centrifuged. Preferably, the sample is centrifuged at about 500 g for about 5 min, and the sample is then resuspended in a second buffer comprising a buffering agent and an ionic strength between 100 mM and 200 mM. Optionally, the second buffer comprises protease inhibitors. In some embodiments, the second buffer is ST, optionally comprising protease inhibitors.

In some embodiments, the method may further comprise isolating nuclei or cell types by FACS sorting.

In some embodiments, the method may further comprise reversing cross-linking in the tissue sample before or during any step of the method. In some embodiments, reversing cross-linking may comprise proteinase digestion. In some embodiments, the proteinase is proteinase K or a cold-active protease.

In some embodiments, the method may further comprise adding a reagent that stabilizes RNA to the tissue sample before or during any step of the method.

In some embodiments, the method may further comprise lysing recovered cells or nuclei and performing reverse transcription, as described in more detail further below.

In specific embodiments, the reverse transcription is performed in individual reaction vessels.

The individual reaction vessel may be an individual discrete volume. An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in our through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

In specific embodiments, the individual reaction vessels may be wells, chambers, or droplets.

Single Cell and Single Nuclei Sequencing

In some embodiments, the method may further comprise performing single cell, single nucleus or bulk RNA-seq, DNA-seq, ATAC-seq, or ChIP on the recovered nuclei or whole cells.

In certain embodiments, the single nuclei and cells according to the present invention are used to generate a single nuclei or single cell sequencing library. The sequencing library may be generated according to any methods known in the art. Non-limiting examples are provided herein.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

In certain embodiments, single cell expression profiling comprises single nucleus RNA sequencing. Single nucleus RNA sequencing advantageously provides for expression profiling of rare or hard to isolate cells. Additionally, single nucleus RNA sequencing may be used on fixed or frozen tissues. The ability of single nucleus sequencing to be performed on frozen tissues allows for the analysis of archived samples isolated from diseased tissues. RNA recovery from previous single nuclei sequencing methods is robust enough for measuring single cell gene expression, however, increased RNA recovery can allow increase gene reads per single cell. Applicants have unexpectedly determined that single nuclei comprising a portion of the rough endoplasmic reticulum (RER) can be isolated and the resulting nuclei provides for improved RNA recovery and single cell expression profiling. In some embodiments, the methods provide for isolation of single nuclei with partially intact outer membrane containing RER. In some embodiments, the methods allow for isolation of single nuclei with partially intact outer membrane and partially intact RER with ribosomes. In some embodiments, the methods allow for isolation of single nuclei with partially intact outer membrane, RER and mitochondria.

In certain embodiments, the present invention provides for a method of single cell sequencing comprising: extracting nuclei from a population of cells under conditions that preserve a portion of the outer nuclear envelope and/or rough endoplasmic reticulum (RER); sorting single nuclei into separate reaction vessels (discrete volumes); extracting RNA from the single nuclei; generating a cDNA library; and sequencing the library, whereby gene expression data from single cells is obtained. As used herein, the term “discrete volume” refers to any reaction volume, vessel, chamber, or the like capable of separating one object from another (e.g., single cell, single nuclei, single bead. Non-limiting examples of discrete volumes include droplets (e.g., emulsion droplets), wells in a plate, or microfluidic chambers.

In certain embodiments, extracting nuclei under conditions that preserve a portion of the outer nuclear envelope and rough endoplasmic reticulum (RER) comprises chopping, homogenizing or grinding the population of cells in a lysis buffer comprising: a detergent selected from the group consisting of NP40, CHAPS and Tween-20; and an ionic strength between 100 mM and 200 mM. The NP40 concentration may be about 0.2%. The Tween-20 concentration may be about 0.03%. The CHAPS concentration may be about 0.49%. In some embodiments, polyamines may be included. Non-limiting examples of chopping include cutting with scissors, chopping with a scalpel or any blade known in the art. Chopping may be manual. Chopping may use any device known in the art capable of chopping.

In certain embodiments, the population of cells may be treated with a reagent that stabilizes RNA. The reagent that stabilizes RNA may be a reagent that comprises the properties of RNAlater™.

In certain embodiments, the separate reaction vessels may be microwells in a plate, as described elsewhere herein. In certain embodiments, the separate reaction vessels may be microfluidic droplets.

Applicants developed microfluidic devices and protocols that allow Drop-seq analysis of thousands of isolated nuclei (Dronc-Seq) (see, e.g., Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239). Furthermore, Applicants have recently made important progress with reverse emulsion devices used for other nuclei-based molecular biology applications, such as a droplet version of single-cell ATAC-Seq. The methods can be applied to single nuclei extracted from tissue samples (e.g., FFPE and frozen tissues). To develop Dronc-Seq Applicants combined the nuclei preparation protocol of Nuc-Seq, a new device compatible with nuclei separation, and Drop-Seq reagents (barcoded beads, molecular biology protocols, lysis buffers) for the in-drop and subsequent phases of the protocol. Briefly, as in Nuc-Seq, Applicants used the published (Sweich et al., 2015) protocols for high quality generation of nuclei suspensions from mouse hippocampus. Unlike Nuc-Seq, where Applicants next sort single nuclei using FACS, in Dronc-Seq Applicants use a microfluidics device, following on the design principles of Drop-Seq, but optimized for the size and properties of nuclei. The nuclei are lysed in drops, and their mRNA captured on the Drop-Seq beads. Notably, given the smaller quantity of mRNA in nuclei, ensuring efficient capture is key. A complementary modality (Klein et al., 2015) has higher capture but lower throughput than Drop-Seq. Finally, Applicants test for cross-contamination due to ‘sticky’ RNA from the lysed cytoplasms or leakage from nuclei using the cross-species controls developed for Drop-Seq (Macosko et al., 2015). Nuclei can also be sorted through FACS prior to Drop-Seq encapsulation. Applicants can also use pore-blocking polymers called poloxamers, such as F-68 and F-127 (Sengupta et al.,2015). Applicants can use Dronc-Seq in the hippocampal biological system and compare to the available of Nuc-Seq benchmarking data. Applicants can also generate Nuc-Seq and Dronc-Seq data from the retina, demonstrating its generality.

In some embodiments, the method may further comprise staining the recovered cells or nuclei using any suitable staining methods known in the art. In specific embodiments, the stain comprises ruby stain.

Methods of Recovering Nuclei and Attached Ribosomes from a Tissue Sample

In some embodiments, the invention provides for methods of recovering nuclei and attached ribosomes from a tissue sample comprising chopping the tissue sample at between 0-4° C. in a nuclear extraction buffer comprising Tris buffer, a detergent and salts; and filtering the sample through a filter between 30-50 uM, preferably 40 uM, and optionally washing the filter with fresh nuclear extraction buffer, wherein the nuclei are present in the supernatant passed through the filter.

As described elsewhere herein, the buffer may comprise a detergent concentration that preserves a portion of the outer nuclear envelope and/or ribosomes, and/or rough endoplasmic reticulum (RER). The detergent may be an ionic, zwitterionic or nonionic detergent. The detergent concentration may be a concentration that is sufficient to lyse cells, but not strong enough to fully dissociate the outer nuclear membrane and RER or detach ribosomes. In certain embodiments, the detergent is selected from the group consisting of NP40, CHAPS and Tween-29. Detergent concentrations may be selected based on the critical micelle concentration (CMC) for each detergent (Table 1). The concentration may be varied above and below the CMC. In certain embodiments, the detergent concentration in the lysis buffer of the present invention comprises about 0.2% NP40, about 0.49% CHAPS, or about 0.03% Tween-20. The critical micelle concentration (CMC) is defined as the concentration of surfactants above which micelles form and all additional surfactants added to the system go to micelles. Before reaching the CMC, the surface tension changes strongly with the concentration of the surfactant. After reaching the CMC, the surface tension remains relatively constant or changes with a lower slope.

In some embodiments, the nuclear extraction buffer comprises 10-20 mM Tris, about 0.49% CHAPS, a salt concentration having an ionic strength of 100-250 mM, and about 0.01% BSA, whereby nuclei are recovered that have a preserved nuclear envelope and ribosomes.

In some embodiments, the nuclear extraction buffer is buffer CST.

In some embodiments, the nuclear extraction buffer comprises 10-20 mM Tris, about 0.03% Tween-20, a salt concentration having an ionic strength of 100-250 mM, and about 0.01% BSA, whereby nuclei are recovered that have a preserved nuclear envelope, rough ER and ribosomes.

In some embodiments, the nuclear extraction buffer is buffer TST.

In some embodiments, the salts comprise 146 mM NaCl, 1 mM CaCl2, and 21 mM MgCl2.

As described elsewhere herein, chopping may comprise chopping with scissors for 1-10 minutes.

In some embodiments, nuclei from specific cell types are genetically modified to express a detectable label on the nuclear membrane and the method further comprises enriching nuclei from the specific cell types using the detectable label.

In some embodiments, the method may further comprise staining the recovered nuclei. In some embodiments, the stain comprises ruby stain.

In some embodiments, the nuclei may be sorted into discrete volumes by FACS, as described elsewhere herein.

In some embodiments, the method may further comprise pelleting the nuclei and resuspending the nuclei in a second buffer consisting of Tris buffer and salts. In some embodiments, the second buffer is buffer ST.

In some embodiments, the method may further comprise generating a single nucleus barcoded library for the recovered nuclei, wherein the nucleic acid from each nucleus is labeled with a barcode sequence comprising a cell of origin barcode, optionally the barcode sequence includes a cell of origin barcode and a unique molecular identifier (UMI).

The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single transcript (e.g., mRNA) or target nucleic acid to be sequenced. Each clone amplified will have a different random UMI that will indicate that the amplified product originated from that clone. The UMI may also be used to determine the number of transcripts that gave rise to an amplified product, or in the case of target barcodes, the number of binding events. In preferred embodiments, the amplification is by PCR or multiple displacement amplification (MDA).

In certain embodiments, reverse transcription (RT) is used to label RNA from single cells or single nuclei with a cell of origin barcode, preferably, a cell of origin barcode and unique molecular identifier (UMI). The barcode may be included on a barcoded RT primer. The primer may also include a capture sequence (e.g., poly T sequence). Thus, the present invention may include barcoding.

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin or individual transcript. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, single nuclei, a viral vector, labeling ligand (e.g., antibody or aptamer), protein, shRNA, sgRNA or cDNA such that multiple species can be sequenced together. Exemplary barcodes may be sequences including but not limited to, TTGAGCCT, AGTTGCTT, CCAGTTAG, ACCAACTG, GTATAACA or CAGGAGCC.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell or nuclei.

The invention provides a mixture comprising a plurality of nucleotide- or oligonucleotide-adorned beads, wherein said beads comprises: a linker; an identical sequence for use as a sequencing priming site; a uniform or near-uniform nucleotide or oligonucleotide sequence; a Unique Molecular Identifier (UMI) which differs for each priming site; an oligonucleotide redundant sequence for capturing polyadenylated mRNAs and priming reverse transcription; and optionally at least one additional oligonucleotide sequences, which provide substrates for downstream molecular-biological reactions; wherein the uniform or near-uniform nucleotide or oligonucleotide sequence is the same across all the priming sites on any one bead, but varies among the oligonucleotides on an individual bead.

In some embodiments, RNA and/or DNA is labeled with the barcode sequence.

In some embodiments, the library is an RNA-seq, DNA-seq, and/or ATAC-seq library, as described elsewhere herein.

In some embodiments, the method may further comprise sequencing the library.

In some embodiments, the tissue sample is fresh frozen.

Nuclei Purification Protocol from Frozen Tissue

In certain embodiments, nuclei extracted from FFPE tissues is compared to nuclei extracted from frozen tissue. Nuclei purification protocol (see., e.g., Swiech L, et al., Nat Biotechnol. 2015 January; 33(1):102-6. doi: 10.1038/nbt.3055. Epub 2014 Oct. 19). The protocol may be modified by using the lysis buffer as described above. In certain embodiments, the procedure may be used for frozen/fixed tissue.

1. Dounce homogenize tissue in 2 ml of ice-cold lysis buffer (25 times with a, 25 times with b), transfer to a 15 ml tube.

1. Rinse homogenizer with 2 ml of ice-cold lysis buffer to get final 4 ml, and collect in the same tube.

2. Mix well and set on ice for 5 minutes.

3. Collect the nuclei by centrifugation at 500×g for 5 minutes at 4° C. Carefully aspirate the clear supernatant from each tube and set the nuclei pellet on ice. Note: The supernatant contains cytoplasmic components and can be saved for later analysis or use.

4. Resuspend. Add 1 ml cold lysis buffer and mix by pipetting gently with a lml tip to completely suspend nuclei pellet. Add the remaining 3 ml of lysis buffer, mix well and set on ice for 5 minutes.

5. Collect washed nuclei by centrifugation as in step 3. Carefully aspirate the clear supernatant and set the nuclei pellet on ice.

6. Optional: Wash. Resuspend in 4 ml 0.01% PBS BSA or Resuspension buffer (RB*). Collect washed nuclei by centrifugation as in step 3.

7. Resuspend with ˜500 μl Resuspension buffer (RB*) or 0.01% PBS BSA+RNAse inhibitor carefully by slow vortex & pipette 10× with a lml tip, then transfer to tubes (for FACS, filter through a membrane to get better purity.

8. Counterstain nuclei with Ruby Dye 1:500-1:1000 (check for clumps in the microscope before sorting).

TABLE 3
Resuspension buffer- based on the original nuclei
resuspension buffer from Swiech et al. 2015:

| | Stocks | For 10 ml | |
|---|---|---|---|
| 340 mM Sucrose | 1M | 3.4 | ml |
| 2 mM MgCl2 | 1M | 10 | ul |
| 25 mM KCl | 2M | 125 | ul |
| 65 mM glycerophosphate | 1M | 650 | ul |
| 5% glycerol | 100% | 500 | ul |

In certain embodiments, nuclei extracted according to any method described herein may be isolated by sucrose gradient centrifugation as described (Swiech L, et al. Nat Biotechnol. 2015 January; 33(1):102-6).

In some embodiments, the tissue sample comprises cells originating from the central nervous system (CNS) or enteric nervous system (ENS). In some embodiments, the tissue sample is obtained from the gut or the brain. In some embodiments, the tissue sample is obtained from a subject suffering from a disease.

In some embodiments, the tissue sample is treated with a reagent that stabilizes RNA.

In some embodiments, the discrete volumes may be droplets, wells in a plate, or microfluidic chambers, as described elsewhere herein.

### Methods of Treating Diseases

The invention also provides a method of treating a disease selected from the group consisting of Hirschsprung's disease (HSCR), inflammatory bowel disease (IBD), autism spectrum disorder (ASD), Parkinson's disease (PD) and schizophrenia in a subject in need thereof. The method comprises administering one or more agents capable of modulating the function or activity of one or more neurons selected from the group consisting of PEMN1, PEMN2, PIMN1, PIMN2, PIMN3, PIMN4, PIMN5, PIN1, PIN2, PSN and PSVN, or one or more cells functionally interacting with the one or more neurons.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

Modulating Agents

In certain embodiments, the present invention provides for one or more therapeutic agents against combinations of targets identified. Targeting the identified genes or cells may provide for enhanced or otherwise previously unknown activity in the treatment of disease. In certain embodiments, an agent against one of the targets may already be known or used clinically. In certain embodiments, a combination therapy may require less of the agent as compared to the current standard of care and provide for less toxicity and improved treatment. In certain embodiments, the agents are used to modulate cell types. For example, the agents may be used to modulate cells for adoptive cell transfer. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

In certain embodiments, the one or more modulating agents may be a genetic modifying agent. The genetic modifying agent may comprise a CRISPR system, a zinc finger nuclease system, a TALEN, a meganuclease or RNAi system.

CRISPR Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EFla promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromouridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine (5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of the guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2,4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O2 concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline<15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/r52), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 mu duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease (also referred to as Cas13a). The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present invention may also use a Cas12 CRISPR enzyme. Cas12 enzymes include Cas12a (Cpf1), Cas12b (C2c1), and Cas12c (C2c3), described further herein.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.
Gaudelli et al. “Programmable base editing of A-T to G-C in genomic DNA without DNA cleavage” Nature 464(551); 464-471 (2017).
   each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA: Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayyeh et al. 2016, Science, 5;353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided Fold Nucleases that recognize extended sequences and can edit endogenous genes with high efficiencies in human cells.

Also, Harrington et al. “Programmed DNA destruction by miniature CRISPR-Cas14 enzymes” Science 2018 doi:10/1126/science.aav4293, relates to Cas14.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, 62/096,324, 23 Dec. 14, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appin cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appin cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9;153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly, these polypeptides can be used for the delivery of CRISPR-effector based RNPs in eukaryotic cells.

Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

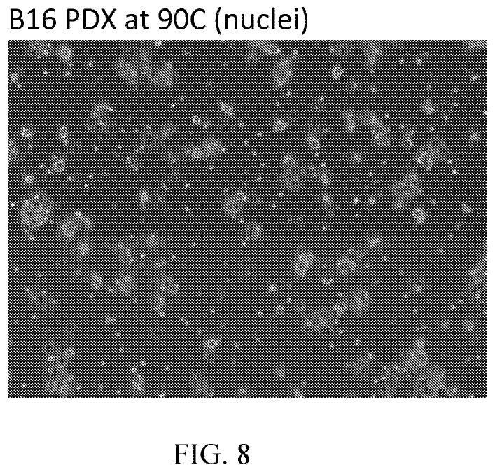

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ. I.D. No. 1)
M D P I R S R T P S P A R E L L S G P Q P D
G V Q P T A D R G V S P P A G G P L D G L P
A R R T M S R T R L P S P P A P S P A F S A
D S F S D L L R Q F D P S L F N T S L F D S
L P P F G A H H T E A A T G E W D E V Q S G
L R A A D A P P P T M R V A V T A A R P P R
A K P A P R R R A A Q P S D A S P A A Q V D
L R T L G Y S Q Q Q Q E K I K P K V R S T V
A Q H H E A L V G H G F T H A H I V A L S Q
H P A A L G T V A V K Y Q D M I A A L P E A
T H E A I V G V G K Q W S G A R A L E A L L
T V A G E L R G P P L Q L D T G Q L L K I A
K R G G V T A V E A V H A W R N A L T G A P
L N

An exemplary amino acid sequence of a C-terminal capping region is:

(SEQ. I.D. No. 2)
R P A L E S I V A Q L S R P D P A L A A L T
N D H L V A L A C L G G R P A L D A V K K G
L P H A P A L I K R T N R R I P E R T S H R
V A D H A Q V V R V L G F F Q C H S H P A Q
A F D D A M T Q F G M S R H G L L Q L F R R
V G V T E L E A R S G T L P P A S Q R W D R
I L Q A S G M K R A K P S P T S T Q T P D Q
A S L H A F A D S L E R D L D A P S P M H E
G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

ZN-Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21 , 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

Antibodies

In certain embodiments, the one or more agents is an antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×107 M-1 (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-0-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

In some embodiments, the one or more cells functionally interacting with the one or more neurons are selected from the group consisting of T cells, dendritic cells (DC), B cells, fibroblasts and adipocytes.

### Methods of Modulating Appetite and Energy Metabolism

In some embodiments, the invention also provides a method of modulating appetite and energy metabolism in a subject in need thereof comprising administering one or more agents capable of modulating the function or activity of one or more neurons selected from the group consisting of PIMN4 and PIMN5; or one or more adipose cells functionally interacting with the one or more neurons.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

In some embodiments, the one or more neurons may be characterized by expression of one or more markers according to Table 14 or Table 21.

In some embodiments, the one or more agents modulate the expression, activity or function of one or more genes according to Table 14 or Table 21.

In some embodiments, the one or more agents may modulate the expression, activity or function of one or more genes selected from the group consisting of: NPY, CGRP, Glutamate, GABA, LEP, VIP, PACAP, Nitric oxide, NOS1, FGF1, PDGF, SLIT2, SLIT3, IL15, IL7, IL12A, PENK, CHAT and TPH2; or NPYR1, CALCRL, GRM8, GABRE, LEPR, VIPR2, GRIA4, GUCY1A3, FGFR1, PDGFRB, ROBO1, ROBO2, IL15R, IL7R, IL12RB1, OPRM1, CHRNE and HTR3A.

In some embodiments, the one or more agents may modulate the expression, activity or function of one or more genes selected from the group consisting of NPY and CGRP; or NPYR1 and CALCRL.

In some embodiments, the one or more agents may modulate the expression, activity or function of one or more core transcriptional programs according to Table 23.

In some embodiments, the one or more agents may modulate the expression, activity or function of one or more genes of the one or more core transcriptional programs.

In some embodiments, the one or more agents are administered to the gut.

In some embodiments, the one or more agents may comprise an antibody, small molecule, small molecule degrader, genetic modifying agent, nucleic acid agent, antibody-like protein scaffold, aptamer, protein, or any combination thereof, as described elsewhere herein.

In some embodiments, the genetic modifying agent may comprise a CRISPR system, RNAi system, a zinc finger nuclease system, a TALE, or a meganuclease, as described above.

In specific embodiments, the CRISPR system comprises Cas9, Cas12, or Cas14.

In specific embodiments, the CRISPR system comprises a dCas fused or otherwise linked to a nucleotide deaminase. The nucleotide deaminase may be a cytidine deaminase or an adenosine deaminase. The dCas may be a dCas9, dCas12, dCas13, or dCas14.

In some embodiments, the nucleic acid agent or genetic modifying agent may be administered with a vector.

In some embodiments, the nucleic acid agent or genetic modifying agent may be under the control of a promoter specific to a marker gene for the one or more neurons according to Table 14 or Table 21.

### Methods of Detecting Cells of the Enteric Nervous System (ENS)

In some embodiments, the invention provides a method of detecting one or more cells of the enteric nervous system (ENS) comprising detecting one or more markers according to Tables 14-17 or Tables 20-22.

Biomarkers

The invention provides biomarkers for the identification, diagnosis and manipulation of cell properties, for use in a variety of diagnostic and/or therapeutic indications. Biomarkers in the context of the present invention encompasses, without limitation nucleic acids, together with their polymorphisms, mutations, variants, modifications, subunits, fragments, and other analytes or sample-derived measures.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

These biomarkers are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The present invention also may comprise a kit with a detection reagent that binds to one or more biomarkers.

In one embodiment, the signature genes, biomarkers, and/or cells may be detected or isolated by immunofluorescence, immunohistochemistry, fluorescence activated cell sorting (FACS), mass cytometry (CyTOF), RNA-seq, scRNA-seq (e.g., Drop-seq, InDrop, 10× Genomics), single cell qPCR, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

Gene Signatures

As used herein a “signature” may encompass any gene or genes, protein or proteins (e.g., gene products), or epigenetic element(s) whose expression profile or whose occurrence is associated with a specific cell type, subtype, or cell state of a specific cell type or subtype within a population of cells (e.g., neurogenic cell). In certain embodiments, the signature is dependent on epigenetic modification of the genes or regulatory elements associated with the genes (e.g., methylation, ubiquitination). Thus, in certain embodiments, use of signature genes includes epigenetic modifications that may be detected or modulated. For ease of discussion, when discussing gene expression, any of gene or genes, protein or proteins, or epigenetic element(s) may be substituted. As used herein, the terms “signature”, “expression profile”, “transcription profile” or “expression program” may be used interchangeably. It is to be understood that also when referring to proteins (e.g. differentially expressed proteins), such may fall within the definition of “gene” signature. Levels of expression or activity may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and/or down-regulated genes that are representative of a cell type or subtype. A gene signature as used herein, may also refer to any set of up- and/or down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. nervous tissue), thus allowing the discovery of novel cell subtypes or cell states that were previously invisible or unrecognized, for example, adult newborn neurons. The presence of subtypes or cell states may be determined by subtype specific or cell state specific signatures. The presence of these specific cell (sub)types or cell states may be determined by applying the signature genes to bulk sequencing data in a sample. The signatures of the present invention may be microenvironment specific, such as their expression in a particular spatio-temporal context. In certain embodiments, signatures as discussed herein are specific to a particular developmental stage or pathological context. In certain embodiments, a combination of cell subtypes having a particular signature may indicate an outcome. The signatures may be used to deconvolute the network of cells present in a particular developmental stage or pathological condition. The presence of specific cells and cell subtypes may also be indicative of a particular developmental stage, a particular response to treatment, such as including increased or decreased susceptibility to treatment. The signature may indicate the presence of one particular cell type. In one embodiment, the novel signatures are used to detect multiple cell states or hierarchies that occur in subpopulations of cells that are linked to particular stages of development or particular pathological condition, or linked to a particular outcome or progression of the disease, or linked to a particular response to treatment of the disease (e.g. resistance to therapy).

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different neurogenic cells, for example, neuronal stem cells, neuronal precursor cells, neuroblasts, immature neurons and newborn neurons, as well as comparing immune cells or immune cell (sub)populations with other immune cells or immune cell (sub)populations. It is to be understood that “differentially expressed” genes/proteins include genes/proteins which are up- or down-regulated as well as genes/proteins which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., proliferating) which can be distinguished or are uniquely identifiable and set apart from other cells of this cell type. The cell subpopulation may be phenotypically characterized, and is preferably characterized by the signature as discussed herein. A cell (sub)population as referred to herein may constitute a (sub)population of cells of a particular cell type characterized by a specific cell state.

When referring to induction, or alternatively reducing or suppression of a particular signature, preferable is meant induction or alternatively reduction or suppression (or upregulation or downregulation) of at least one gene/protein and/or epigenetic element of the signature, such as for instance at least two, at least three, at least four, at least five, at least six, or all genes/proteins and/or epigenetic elements of the signature.

Various aspects and embodiments of the invention may involve analyzing gene signatures, protein signatures, and/or other genetic or epigenetic signatures based on single cell analyses (e.g. single cell RNA sequencing) or alternatively based on cell population analyses, as is defined herein elsewhere.

The invention further relates to various uses of the gene signatures, protein signature, and/or other genetic or epigenetic signature as defined herein. Particular advantageous uses include methods for identifying agents capable of inducing or suppressing neurogenesis, particularly inducing or suppressing neurogenic cell(sub)populations based on the gene signatures, protein signature, and/or other genetic or epigenetic signature as defined herein. The invention further relates to agents capable of inducing or suppressing particular neurogenic cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular gene signature, protein signature, and/or other genetic or epigenetic signature may modulate neurogenesis, and/or neurogeneic cell subpopulation composition or distribution, or functionality.

The signature genes of the present invention were discovered by analysis of expression profiles of single-cells within a population of neurogenic cells, thus allowing the discovery of novel cell subtypes that were previously invisible or rare in a population of cells within the nervous tissue. The presence of subtypes may be determined by subtype specific signature genes. The presence of these specific cell types may be determined by applying the signature genes to bulk sequencing data in a patient. Not being bound by a theory, many cells make up a microenvironment, whereby the cells communicate and affect each other in specific ways. As such, specific cell types within this microenvironment may express signature genes specific for this microenvironment. Not being bound by a theory the signature genes of the present invention may be microenvironment specific. The signature genes may indicate the presence of one particular cell type. In one embodiment, the expression may indicate the presence of proliferating cell types. Not being bound by a theory, a combination of cell subtypes in a subject may indicate an outcome.

As used herein the term “biological program” can be used interchangeably with “expression program” or “transcriptional program” and may refer to a set of genes that share a role in a biological function (e.g., an activation program, cell differentiation program, proliferation program). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor.

All gene name symbols refer to the gene as commonly known in the art. The examples described herein that refer to the mouse gene names are to be understood to also encompasses human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. The signature as described herein may encompass any of the genes described herein.

In specific embodiments, detecting the one or more markers comprises immunohistochemistry.

### Methods of Screening

The invention also provides for methods of screening for agents capable of modulating expression of a transcription program according to Table 23. Such methods may comprise administering an agent to a population of cells comprising neurons selected from the group consisting of PEMN1, PEMN2, PIMN1, PIMN2, PIMN3, PIMN4, PIMN5, PIN1, PIN2, PSN and PSVN; and detecting expression of one or more genes in the transcriptional program.

Screening for Modulating Agents

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures of the present invention may be used to screen for drugs that reduce the signature in cells as described herein. The signature may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature of the present invention in silico.

In some embodiments, detecting expression comprises RT-PCR, RNA-seq, single cell RNA-seq, fluorescently labeled probes, or an immunoassay, as described elsewhere herein.

In some embodiments, the neurons express one or more reporter genes under control of a promoter specific to the one or more genes in the transcriptional program. In some embodiments, detecting comprises detecting the reporter gene.

### Methods of Identifying Gene Expression in Single Cells

The invention also provides a method of identifying gene expression in single cells comprising providing sequencing reads from a single nucleus sequencing library and counting sequencing reads mapping to introns and exons.

Microfluidics

In a preferred embodiment, single cell or single nuclei analysis is performed using microfluidics. Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets. The ability to conduct reactions in microdroplets depends on being able to merge different sample fluids and different microdroplets. See, e.g., US Patent Publication No. 20120219947 and PCT publication No. WO2014085802 A1.

Droplet microfluidics offers significant advantages for performing high-throughput screens and sensitive assays. Droplets allow sample volumes to be significantly reduced, leading to concomitant reductions in cost. Manipulation and measurement at kilohertz speeds enable up to 108 samples to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

The manipulation of fluids to form fluid streams of desired configuration, discontinuous fluid streams, droplets, particles, dispersions, etc., for purposes of fluid delivery, product manufacture, analysis, and the like, is a relatively well-studied art. Microfluidic systems have been described in a variety of contexts, typically in the context of miniaturized laboratory (e.g., clinical) analysis. Other uses have been described as well. For example, WO 2001/89788; WO 2006/040551; U.S. Patent Application Publication No. 2009/0005254; WO 2006/040554; U.S. Patent Application Publication No. 2007/0184489; WO 2004/002627; U.S. Pat. No. 7,708,949; WO 2008/063227; U.S. Patent Application Publication No. 2008/0003142; WO 2004/091763; U.S. Patent Application Publication No. 2006/0163385; WO 2005/021151; U.S. Patent Application Publication No. 2007/0003442; WO 2006/096571; U.S. Patent Application Publication No. 2009/0131543; WO 2007/089541; U.S. Patent Application Publication No. 2007/0195127; WO 2007/081385; U.S. Patent Application Publication No. 2010/0137163; WO 2007/133710; U.S. Patent Application Publication No. 2008/0014589; U.S. Patent Application Publication No. 2014/0256595; and WO 2011/079176. In a preferred embodiment, single cell analysis is performed in droplets using methods according to WO 2014085802. Each of these patents and publications is herein incorporated by reference in their entireties for all purposes.

Single cells or nuclei may be sorted into separate vessels by dilution of the sample and physical movement, such as micromanipulation devices or pipetting. A computer controlled machine may control pipetting and separation.

Single cells or single nuclei of the present invention may be divided into single droplets using a microfluidic device. The single cells or nuclei in such droplets may be further labeled with a barcode. In this regard reference is made to Macosko et al., 2015, “Highly Parallel Genome-wide Expression Profiling of Individual Cells Using Nanoliter Droplets” Cell 161, 1202-1214 and Klein et al., 2015, “Droplet Barcoding for Single-Cell Transcriptomics Applied to Embryonic Stem Cells” Cell 161, 1187-1201 all the contents and disclosure of each of which are herein incorporated by reference in their entirety. Not being bound by a theory, the volume size of an aliquot within a droplet may be as small as 1 fL

Single cells or single nuclei may be diluted into a physical multi-well plate or a plate free environment. The multi-well assay modules (e.g., plates) may have any number of wells and/or chambers of any size or shape, arranged in any pattern or configuration, and be composed of a variety of different materials. Preferred embodiments of the invention are multi-well assay plates that use industry standard multi-well plate formats for the number, size, shape and configuration of the plate and wells. Examples of standard formats include 96-, 384-, 1536- and 9600-well plates, with the wells configured in two-dimensional arrays. Other formats include single well, two well, six well and twenty-four well and 6144 well plates. Plate free environments of the present invention utilize a single polymerizable gel containing compartmentalized cells or single nuclei. In one embodiment, extraction of single cells or single nuclei may be by a mechanical punch. Single cells or single nuclei may be visualized in the gel before a punch.

In one embodiment, to ensure proper staining of intracellular and intranuclear proteins and nucleic acids single cells or nuclei are embedded in hydrogel droplets. Not being bound by a theory, the hydrogel mesh provides a physical framework, chemically incorporates biomolecules and is permeable to macromolecules such as antibodies (Chung et al., (2013). Structural and molecular interrogation of intact biological systems. Nature 497, 332-337). In one embodiment, to further improve permeability and staining efficiency, lipids are cleared (Chung et al., 2013). Not being bound by a theory, the clearance of the lipids and the porosity of the hydrogel allow for more efficient washing. This higher accuracy of measurement is important for the high multiplex measurements and computational inference of regulatory mechanisms.

In one embodiment, the nucleic acids of single cells or nuclei are crosslinked to prevent loss of nucleic acids. Not being bound by a theory, leakage of mRNA from nuclei may be prevented by crosslinking. Nucleic acids can be reverse cross-linked after separation of cells or nuclei into separate wells or droplets. The contents of individual wells or droplets may then be sequenced. In one embodiment, crosslinking may be reversed by incubating the cross-linked sample in high salt (approximately 200 mM NaCl) at 65° C. for at least 4 h.

The invention provides a nucleotide- or oligonucleotide-adorned bead wherein said bead comprises: a linker; an identical sequence for use as a sequencing priming site; a uniform or near-uniform nucleotide or oligonucleotide sequence (e.g., each bead has a barcode sequence that is unique to each bead in a plurality of beads); a Unique Molecular Identifier which differs for each priming site; optionally an oligonucleotide redundant sequence for capturing polyadenylated mRNAs and priming reverse transcription; and optionally at least one other oligonucleotide barcode which provides an additional substrate for identification.

In an embodiment of the invention, the nucleotide or oligonucleotide sequences on the surface of the bead is a molecular barcode. In a further embodiment, the barcode ranges from 4 to 1000 nucleotides in length. In another embodiment, the oligonucleotide sequence for capturing polyadenylated mRNAs and priming reverse transcription is an oligo dT sequence.

In an embodiment of the invention, the linker is a non-cleavable, straight-chain polymer. In another embodiment, the linker is a chemically-cleavable, straight-chain polymer. In a further embodiment, the linker is a non-cleavable, optionally substituted hydrocarbon polymer. In another embodiment, the linker is a photolabile optionally substituted hydrocarbon polymer. In another embodiment, the linker is a polyethylene glycol. In an embodiment, the linker is a PEG-C3 to PEG-24.

In an embodiment of the invention, the nucleotide or oligonucleotide sequence on the surface of the bead is a molecular barcode. In a further embodiment, the barcode ranges from 4 to 1000 nucleotides in length. In another embodiment, the oligonucleotide sequence for capturing polyadenylated mRNAs and priming reverse transcription is an oligo dT sequence.

In an embodiment of the invention, the mixture comprises at least one oligonucleotide sequences, which provide for substrates for downstream molecular-biological reactions. In another embodiment, the downstream molecular biological reactions are for reverse transcription of mature mRNAs; capturing specific portions of the transcriptome, priming for DNA polymerases and/or similar enzymes; or priming throughout the transcriptome or genome. In an embodiment of the invention, the additional oligonucleotide sequence comprises an oligo-dT sequence. In another embodiment of the invention, the additional oligonucleotide sequence comprises a primer sequence. In an embodiment of the invention, the additional oligonucleotide sequence comprises an oligo-dT sequence and a primer sequence.

The invention provides an error-correcting barcode bead wherein said bead comprises: a linker; an identical sequence for use as a sequencing priming site; a uniform or near-uniform nucleotide or oligonucleotide sequence which comprises at least a nucleotide base duplicate; a Unique Molecular Identifier which differs for each priming site; and an oligonucleotide redundant for capturing polyadenylated mRNAs and priming reverse transcription.

In an embodiment of the invention, the error-correcting barcode beads fail to hybridize to the mRNA thereby failing to undergo reverse transcription.

The invention also provides a kit which comprises a mixture of oligonucleotide bound beads and self-correcting barcode beads.

The invention provides a method for creating a single-cell sequencing library comprising: merging one uniquely barcoded RNA capture microbead with a single-cell or single nuclei in an emulsion droplet having a diameter from 50 μm to 210 μm; lysing the cell thereby capturing the RNA on the RNA capture microbead; breaking droplets and pooling beads in solution; performing a reverse transcription reaction to convert the cells' RNA to first strand cDNA that is covalently linked to the RNA capture microbead; or conversely reverse transcribing within droplets and thereafter breaking droplets and collecting cDNA-attached beads; preparing and sequencing a single composite RNA-Seq library, containing cell barcodes that record the cell-of-origin of each RNA, and molecular barcodes that distinguish among RNAs from the same cell.

In an embodiment the diameter of the emulsion droplet is between 50-210 μm. In a further embodiment, the method wherein the diameter of the mRNA capture microbeads is from 10 μm to 95 μm. In a further embodiment the diameter of the emulsion droplet is 90 μm.

The invention provides a method for preparing a plurality of beads with unique nucleic acid sequence comprising: performing polynucleotide synthesis on the surface of the plurality of beads in a pool-and-split process, such that in each cycle of synthesis the beads are split into a plurality of subsets wherein each subset is subjected to different chemical reactions; repeating the pool-and-split process from anywhere from 2 cycles to 200 cycles.

In an embodiment of the invention the polynucleotide synthesis is phosphoramidite synthesis. In another embodiment of the invention the polynucleotide synthesis is reverse direction phosphoramidite chemistry. In an embodiment of the invention, each subset is subjected to a different nucleotide. In another embodiment, each subset is subjected to a different canonical nucleotide. In an embodiment of the invention the method is repeated three, four, or twelve times.

In an embodiment the covalent bond is polyethylene glycol. In another embodiment the diameter of the mRNA capture microbeads is from 10 μm to 95 μm. In an embodiment, wherein the multiple steps is twelve steps.

In a further embodiment the method further comprises a method for preparing uniquely barcoded mRNA capture microbeads, which has a unique barcode and diameter suitable for microfluidic devices comprising: 1) performing reverse phosphoramidite synthesis on the surface of the bead in a pool-and-split fashion, such that in each cycle of synthesis the beads are split into four reactions with one of the four canonical nucleotides (T, C, G, or A); 2) repeating this process a large number of times, at least six, and optimally more than twelve, such that, in the latter, there are more than 16 million unique barcodes on the surface of each bead in the pool.

In an embodiment, the diameter of the mRNA capture microbeads is from 10 μm to 95 μm.

The invention provides a method for simultaneously preparing a plurality of nucleotide- or oligonucleotide-adorned beads wherein a uniform, near-uniform, or patterned nucleotide or oligonucleotide sequence is synthesized upon any individual bead while vast numbers of different nucleotide or oligonucleotide sequences are simultaneously synthesized on different beads, comprising: forming a mixture comprising a plurality of beads; separating the beads into subsets; extending the nucleotide or oligonucleotide sequence on the surface of the beads by adding an individual nucleotide via chemical synthesis; pooling the subsets of beads in (c) into a single common pool; repeating steps (b), (c) and (d) multiple times to produce a combinatorially a thousand or more nucleotide or oligonucleotide sequences; and collecting the nucleotide- or oligonucleotide-adorned beads.

In an embodiment of the invention, the nucleotide or oligonucleotide sequence on the surface of the bead is a molecular barcode. In a further embodiment, the pool-and-split synthesis steps occur every 2-10 cycles, rather than every cycle.

In an embodiment of the invention, the barcode contains built-in error correction. In another embodiment, the barcode ranges from 4 to 1000 nucleotides in length. In embodiment of the invention the polynucleotide synthesis is phosphoramidite synthesis. In a further embodiment, the polynucleotide synthesis is reverse direction phosphoramidite chemistry. In an embodiment of the invention each subset is subjected to a different nucleotide. In a further embodiment, one or more subsets receive a cocktail of two nucleotides. In an embodiment, each subset is subjected to a different canonical nucleotide.

The method provided by the invention contemplates a variety of embodiments wherein the bead is a microbead, a nanoparticle, or a macrobead. Similarly, the invention contemplates that the oligonucleotide sequence is a dinucleotide or trinucleotide.

The invention provides a method for simultaneously preparing a thousand or more nucleotide- or oligonucleotide-adorned beads wherein a uniform or near-uniform nucleotide or oligonucleotide sequence is synthesized upon any individual bead while a plurality of different nucleotide or oligonucleotide sequences are simultaneously synthesized on different beads, comprising: forming a mixture comprising a plurality of beads; separating the beads into subsets; extending the nucleotide or oligonucleotide sequence on the surface of the beads by adding an individual nucleotide via chemical synthesis; pooling the subsets of beads in (c) into a single common pool; repeating steps (b), (c) and (d) multiple times to produce a combinatorically large number of nucleotide or oligonucleotide sequences; and collecting the nucleotide- or oligonucleotide-adorned beads; performing polynucleotide synthesis on the surface of the plurality of beads in a pool-and-split synthesis, such that in each cycle of synthesis the beads are split into a plurality of subsets wherein each subset is subjected to different chemical reactions; repeating the pool-and-split synthesis multiple times.

In an embodiment of the invention, the nucleotide or oligonucleotide sequence on the surface of the bead is a molecular barcode. In an embodiment, the pool-and-split synthesis steps occur every 2 to 10 cycles, rather than every cycle. In an embodiment, the generated barcode contains built-in error correction. In another embodiment, the barcode ranges from 4 to 1000 nucleotides in length. In embodiment of the invention the polynucleotide synthesis is phosphoramidite synthesis. In a further embodiment, the polynucleotide synthesis is reverse direction phosphoramidite chemistry. In an embodiment of the invention each subset is subjected to a different nucleotide. In a further embodiment, one or more subsets receive a cocktail of two nucleotides. In an embodiment, each subset is subjected to a different canonical nucleotide.

The method provided by the invention contemplates a variety of embodiments wherein the bead is a microbead, a nanoparticle, or a macrobead. Similarly, the invention contemplates that the oligonucleotide sequence is a dinucleotide or trinucleotide.

The invention further provides an apparatus for creating a composite single-cell sequencing library via a microfluidic system, comprising: an oil-surfactant inlet comprising a filter and two carrier fluid channels, wherein said carrier fluid channel further comprises a resistor; an inlet for an analyte comprising a filter and two carrier fluid channels, wherein said carrier fluid channel further comprises a resistor; an inlet for mRNA capture microbeads and lysis reagent comprising a carrier fluid channel; said carrier fluid channels have a carrier fluid flowing therein at an adjustable and predetermined flow rate; wherein each said carrier fluid channels merge at a junction; and said junction being connected to a constriction for droplet pinch-off followed by a mixer, which connects to an outlet for drops.

In an embodiment of the apparatus, the analyte comprises a chemical reagent, a genetically perturbed cell, a protein, a drug, an antibody, an enzyme, a nucleic acid, an organelle like the mitochondrion or nucleus, a cell or any combination thereof. In an embodiment of the apparatus the analyte is a cell. In a further embodiment, the analyte is a mammalian cell. In another embodiment, the analyte of the apparatus is complex tissue. In a further embodiment, the cell is a brain cell. In an embodiment of the invention, the cell is a retina cell. In another embodiment, the cell is a human bone marrow cell. In an embodiment, the cell is a host-pathogen cell. In an embodiment, the analyte is a nucleus from a cell.

In an embodiment of the apparatus the lysis reagent comprises an anionic surfactant such as sodium lauroyl sarcosinate, or a chaotropic salt such as guanidinium thiocyanate. In an embodiment of the apparatus the filter is consists of square PDMS posts; the filter on the cell channel consists of such posts with sides ranging between 125-135 μm with a separation of 70-100 mm between the posts. The filter on the oil-surfactant inlet comprises square posts of two sizes; one with sides ranging between 75-100 μm and a separation of 25-30 μm between them and the other with sides ranging between 40-50 μm and a separation of 10-15 μm. In an embodiment of the apparatus the resistor is serpentine having a length of 7000-9000 μm, width of 50-75 μm and depth of 100-150 mm. In an embodiment of the apparatus the channels have a length of 8000-12,000 μm for oil-surfactant inlet, 5000-7000 for analyte (cell) inlet, and 900-1200 μm for the inlet for microbead and lysis agent. All channels have a width of 125-250 mm, and depth of 100-150 mm. In another embodiment, the width of the cell channel is 125-250 μm and the depth is 100-150 μm. In an embodiment of the apparatus the mixer has a length of 7000-9000 μm, and a width of 110-140 μm with 35-45° zig-zigs every 150 μm. In an embodiment, the width of the mixer is 125 μm. In an embodiment of the apparatus the oil-surfactant is PEG Block Polymer, such as BIORAD™ QX200 Droplet Generation Oil. In an embodiment of the apparatus the carrier fluid is water-glycerol mixture.

A mixture comprising a plurality of microbeads adorned with combinations of the following elements: bead-specific oligonucleotide barcodes created by the methods provided; additional oligonucleotide barcode sequences which vary among the oligonucleotides on an individual bead and can therefore be used to differentiate or help identify those individual oligonucleotide molecules; additional oligonucleotide sequences that create substrates for downstream molecular-biological reactions, such as oligo-dT (for reverse transcription of mature mRNAs), specific sequences (for capturing specific portions of the transcriptome, or priming for DNA polymerases and similar enzymes), or random sequences (for priming throughout the transcriptome or genome). In an embodiment, the individual oligonucleotide molecules on the surface of any individual microbead contain all three of these elements, and the third element includes both oligo-dT and a primer sequence.

In another embodiment, a mixture comprising a plurality of microbeads, wherein said microbeads comprise the following elements: at least one bead-specific oligonucleotide barcode obtainable by the process outlined; at least one additional identifier oligonucleotide barcode sequence, which varies among the oligonucleotides on an individual bead, and thereby assisting in the identification and of the bead specific oligonucleotide molecules; optionally at least one additional oligonucleotide sequences, which provide substrates for downstream molecular-biological reactions. In another embodiment the mixture comprises at least one oligonucleotide sequences, which provide for substrates for downstream molecular-biological reactions. In a further embodiment the downstream molecular biological reactions are for reverse transcription of mature mRNAs; capturing specific portions of the transcriptome, priming for DNA polymerases and/or similar enzymes; or priming throughout the transcriptome or genome. In a further embodiment the mixture the additional oligonucleotide sequence comprising an oligo-dT sequence. In another embodiment the mixture further comprises the additional oligonucleotide sequence comprises a primer sequence. In another embodiment the mixture further comprises the additional oligonucleotide sequence comprising an oligo-dT sequence and a primer sequence.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

In an advantageous embodiment, agents may be uniquely labeled in a dynamic manner (see, e.g., U.S. provisional patent application Ser. No. 61/703,884 filed Sep. 21, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

The oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the nonnucleic acid detectable moiety.

In some embodiments, a detectable oligonucleotide tag may comprise one or more nonoligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art. In some embodiments, the detectable moieties may be quantum dots. Methods for detecting such moieties are described herein and/or are known in the art.

Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties.

A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag.

In some embodiments, one or more other species may be associated with the tags. In particular, nucleic acids released by a lysed cell may be ligated to one or more tags. These may include, for example, chromosomal DNA, RNA transcripts, tRNA, mRNA, mitochondrial DNA, or the like. Such nucleic acids may be sequenced, in addition to sequencing the tags themselves, which may yield information about the nucleic acid profile of the cells, which can be associated with the tags, or the conditions that the corresponding droplet or cell was exposed to.

The invention described herein enables high throughput and high resolution delivery of reagents to individual emulsion droplets that may contain cells, organelles, nucleic acids, proteins, etc. through the use of monodisperse aqueous droplets that are generated by a microfluidic device as a water-in-oil emulsion. The droplets are carried in a flowing oil phase and stabilized by a surfactant. In one aspect single cells or single organellesor single molecules (proteins, RNA, DNA) are encapsulated into uniform droplets from an aqueous solution/dispersion. In a related aspect, multiple cells or multiple molecules may take the place of single cells or single molecules. The aqueous droplets of volume ranging from 1 pL to 10 nL work as individual reactors. Disclosed embodiments provide thousands of single cells in droplets which can be processed and analyzed in a single run.

To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination.

Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets.

Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. In another embodiment, the expansion and/or contraction of the liquid reservoir may be combined with other flow-controlling devices and methods, e.g., as described herein. Non-limiting examples of devices able to cause the expansion and/or contraction of a liquid reservoir include pistons.

Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel.

Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform.

Droplets formed in oil without surfactant are not stable to permit coalescence, so surfactants must be dissolved in the oil that is used as the continuous phase for the emulsion library. Surfactant molecules are amphiphilic--part of the molecule is oil soluble, and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets.

A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification.

A cell library element may include, but is not limited to, hybridomas, B-cells, primary cells, cultured cell lines, cancer cells, stem cells, cells obtained from tissue (e.g., brain, gut or gastrointestinal, retinal or human bone marrow), peripheral blood mononuclear cell, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element.

A variety of analytes may be contemplated for use with the foregoing Drop-Sequencing methods. Examples of cells which are contemplated are mammalian cells, however the invention contemplates a method for profiling host-pathogen cells. To characterize the expression of host-pathogen interactions it is important to grow the host and pathogen in the same cell without multiple opportunities of pathogen infection.

A bead based library element may contain one or more beads, of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids.

Often it is desirable to have exactly one cell or nuclei per droplet with only a few droplets containing more than one cell or nuclei when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell.

Examples of droplet libraries are collections of droplets that have different contents, ranging from beads, cells, nuclei, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges.

The droplets comprised within the emulsion libraries of the present invention may be contained within an immiscible oil which may comprise at least one fluorosurfactant. In some embodiments, the fluorosurfactant comprised within immiscible fluorocarbon oil is a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays described herein. Fluids (e.g., aqueous fluids, immiscible oils, etc.) and other surfactants that may be utilized in the droplet libraries of the present invention are described in greater detail herein.

The present invention provides an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library.

For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In one example, the cells may comprise cancer cells of a tissue biopsy, and each cell type is encapsulated to be screened for genomic data or against different drug therapies. Another example is that 1011 or 1015 different type of bacteria; each having a different plasmid spliced therein, are encapsulated. One example is a bacterial library where each library element grows into a clonal population that secretes a variant on an enzyme.

In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. In one specific example, a LacZ plasmid DNA was encapsulated at a concentration of 20 fM after two hours of incubation such that there was about one gene in 40 droplets, where 10 μm droplets were made at 10 kHz per second. Formation of these libraries rely on limiting dilutions.

The present invention also provides an emulsion library which may comprise at least a first aqueous droplet and at least a second aqueous droplet within a fluorocarbon oil which may comprise at least one fluorosurfactant, wherein the at least first and the at least second droplets are uniform in size and comprise a different aqueous fluid and a different library element. The present invention also provides a method for forming the emulsion library which may comprise providing at least a first aqueous fluid which may comprise at least a first library of elements, providing at least a second aqueous fluid which may comprise at least a second library of elements, encapsulating each element of said at least first library into at least a first aqueous droplet within an immiscible fluorocarbon oil which may comprise at least one fluorosurfactant, encapsulating each element of said at least second library into at least a second aqueous droplet within an immiscible fluorocarbon oil which may comprise at least one fluorosurfactant, wherein the at least first and the at least second droplets are uniform in size and comprise a different aqueous fluid and a different library element, and pooling the at least first aqueous droplet and the at least second aqueous droplet within an immiscible fluorocarbon oil which may comprise at least one fluorosurfactant thereby forming an emulsion library.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

Size selection of the nucleic acids may be performed to remove very short fragments or very long fragments. The nucleic acid fragments may be partitioned into fractions which may comprise a desired number of fragments using any suitable method known in the art. Suitable methods to limit the fragment size in each fragment are known in the art. In various embodiments of the invention, the fragment size is limited to between about 10 and about 100 Kb or longer.

In another embodiment, the sample includes individual target proteins, protein complexes, proteins with translational modifications, and protein/nucleic acid complexes. Protein targets include peptides, and also include enzymes, hormones, structural components such as viral capsid proteins, and antibodies. Protein targets may be synthetic or derived from naturally-occurring sources. In one embodiment of the invention protein targets are isolated from biological samples containing a variety of other components including lipids, non-template nucleic acids, and nucleic acids. In certain embodiments, protein targets may be obtained from an animal, bacterium, fungus, cellular organism, and single cells. Protein targets may be obtained directly from an organism or from a biological sample obtained from the organism, including bodily fluids such as blood, urine, cerebrospinal fluid, seminal fluid, saliva, sputum, stool and tissue. Protein targets may also be obtained from cell and tissue lysates and biochemical fractions. An individual protein is an isolated polypeptide chain. A protein complex includes two or polypeptide chains. Samples may include proteins with post translational modifications including but not limited to phosphorylation, methionine oxidation, deamidation, glycosylation, ubiquitination, carbamylation, S-carboxymethylation, acetylation, and methylation. Protein/nucleic acid complexes include cross-linked or stable protein-nucleic acid complexes.

Extraction or isolation of individual proteins, protein complexes, proteins with translational modifications, and protein/nucleic acid complexes is performed using methods known in the art.

Methods of the invention involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety.

The sample fluid may typically comprise an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid may include one that is immiscible with the sample fluid. The carrier fluid can be a non-polar solvent, decane (e.g., tetradecane or hexadecane), fluorocarbon oil, silicone oil, an inert oil such as hydrocarbon, or another oil (for example, mineral oil).

In certain embodiments, the carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing.

In certain embodiments, the droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

In certain embodiments, the carrier fluid may be caused to flow through the outlet channel so that the surfactant in the carrier fluid coats the channel walls. In one embodiment, the fluorosurfactant can be prepared by reacting the perfluorinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., Fluorinert (3M)), which then serves as the carrier fluid.

Activation of sample fluid reservoirs to produce regent droplets is now described. The disclosed invention is based on the concept of dynamic reagent delivery (e.g., combinatorial barcoding) via an on demand capability. The on demand feature may be provided by one of a variety of technical capabilities for releasing delivery droplets to a primary droplet, as described herein.

An aspect in developing this device will be to determine the flow rates, channel lengths, and channel geometries. Once these design specifications are established, droplets containing random or specified reagent combinations can be generated on demand and merged with the “reaction chamber” droplets containing the samples/cells/substrates of interest.

By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioninformatically record information can be found at US Provisional Patent Application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional Patent Application entitled “Systems and Methods for Droplet Tagging” filed Sep. 21, 2012.

Applications of the disclosed device may include use for the dynamic generation of molecular barcodes (e.g., DNA oligonucleotides, fluorophores, etc.) either independent from or in concert with the controlled delivery of various compounds of interest (drugs, small molecules, siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with cell-containing droplets. An electronic record in the form of a computer log file is kept to associate the barcode delivered with the downstream reagent(s) delivered. This methodology makes it possible to efficiently screen a large population of cells for applications such as single-cell drug screening, controlled perturbation of regulatory pathways, etc. The device and techniques of the disclosed invention facilitate efforts to perform studies that require data resolution at the single cell (or single molecule) level and in a cost effective manner. Disclosed embodiments provide a high throughput and high resolution delivery of reagents to individual emulsion droplets that may contain cells, nucleic acids, proteins, etc. through the use of monodisperse aqueous droplets that are generated one by one in a microfluidic chip as a water-in-oil emulsion. Hence, the invention proves advantageous over prior art systems by being able to dynamically track individual cells and droplet treatments/combinations during life cycle experiments. Additional advantages of the disclosed invention provides an ability to create a library of emulsion droplets on demand with the further capability of manipulating the droplets through the disclosed process(es). Disclosed embodiments may, thereby, provide dynamic tracking of the droplets and create a history of droplet deployment and application in a single cell based environment. In certain example embodiments, the methods disclosed herein may be used to conduct pooled CRISPR screening such as that disclosed in Datlinger et al. bioRXiv dx.doi.org/10.1101/083774.

Droplet generation and deployment is produced via a dynamic indexing strategy and in a controlled fashion in accordance with disclosed embodiments of the present invention. Disclosed embodiments of the microfluidic device described herein provides the capability of microdroplets that be processed, analyzed and sorted at a highly efficient rate of several thousand droplets per second, providing a powerful platform which allows rapid screening of millions of distinct compounds, biological probes, proteins or cells either in cellular models of biological mechanisms of disease, or in biochemical, or pharmacological assays.

A plurality of biological assays as well as biological synthesis are contemplated for the present invention.

In an advantageous embodiment, polymerase chain reactions (PCR) are contemplated (see, e.g., US Patent Publication No. 20120219947). Methods of the invention may be used for merging sample fluids for conducting any type of chemical reaction or any type of biological assay. In certain embodiments, methods of the invention are used for merging sample fluids for conducting an amplification reaction in a droplet. Amplification refers to production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany F. (1991) PNAS 88:189-193; Barany F. (1991) PCR Methods and Applications 1:5-16), ligase detection reaction (Barany F. (1991) PNAS 88:189-193), strand displacement amplification and restriction fragments length polymorphism, transcription based amplification system, nucleic acid sequence-based amplification, rolling circle amplification, and hyper-branched rolling circle amplification.

In certain embodiments, the amplification reaction is the polymerase chain reaction. Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target sequence includes introducing an excess of oligonucleotide primers to a DNA mixture containing a desired target sequence, followed by a precise sequence of thermal cycling in the presence of a DNA polymerase. The primers are complementary to their respective strands of the double stranded target sequence.

To effect amplification, primers are annealed to their complementary sequence within the target molecule. Following annealing, the primers are extended with a polymerase so as to form a new pair of complementary strands. The steps of denaturation, primer annealing and polymerase extension may be repeated many times (i.e., denaturation, annealing and extension constitute one cycle; there may be numerous cycles) to obtain a high concentration of an amplified segment of a desired target sequence. The length of the amplified segment of the desired target sequence is determined by relative positions of the primers with respect to each other, and therefore, this length is a controllable parameter.

Methods for performing PCR in droplets are shown for example in Link et al. (U.S. Patent application numbers 2008/0014589, 2008/0003142, and 2010/0137163), Anderson et al. (U.S. Pat. No. 7,041,481 and which reissued as RE41,780) and European publication number EP2047910 to Raindance Technologies Inc. The content of each of which is incorporated by reference herein in its entirety.

The first sample fluid contains nucleic acid templates. Droplets of the first sample fluid are formed as described above. Those droplets will include the nucleic acid templates. In certain embodiments, the droplets will include only a single nucleic acid template, and thus digital PCR may be conducted. The second sample fluid contains reagents for the PCR reaction. Such reagents generally include Taq polymerase, deoxynucleotides of type A, C, G and T, magnesium chloride, and forward and reverse primers, all suspended within an aqueous buffer. The second fluid also includes detectably labeled probes for detection of the amplified target nucleic acid, the details of which are discussed below. This type of partitioning of the reagents between the two sample fluids is not the only possibility. In certain embodiments, the first sample fluid will include some or all of the reagents necessary for the PCR whereas the second sample fluid will contain the balance of the reagents necessary for the PCR together with the detection probes.

Primers may be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers may also be obtained from commercial sources such as Operon Technologies, Amersham Pharmacia Biotech, Sigma, and Life Technologies. The primers may have an identical melting temperature. The lengths of the primers may be extended or shortened at the 5′ end or the 3′ end to produce primers with desired melting temperatures. Also, the annealing position of each primer pair may be designed such that the sequence and, length of the primer pairs yield the desired melting temperature. The simplest equation for determining the melting temperature of primers smaller than 25 base pairs is the Wallace Rule (Td=2(A+T)+4(G+C)). Computer programs may also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The TM (melting or annealing temperature) of each primer is calculated using software programs such as Oligo Design, available from Invitrogen Corp.

A droplet containing the nucleic acid is then caused to merge with the PCR reagents in the second fluid according to methods of the invention described above, producing a droplet that includes Taq polymerase, deoxynucleotides of type A, C, G and T, magnesium chloride, forward and reverse primers, detectably labeled probes, and the target nucleic acid.

Once mixed droplets have been produced, the droplets are thermal cycled, resulting in amplification of the target nucleic acid in each droplet. In certain embodiments, the droplets are flowed through a channel in a serpentine path between heating and cooling lines to amplify the nucleic acid in the droplet. The width and depth of the channel may be adjusted to set the residence time at each temperature, which may be controlled to anywhere between less than a second and minutes.

In certain embodiments, the three temperature zones are used for the amplification reaction. The three temperature zones are controlled to result in denaturation of double stranded nucleic acid (high temperature zone), annealing of primers (low temperature zones), and amplification of single stranded nucleic acid to produce double stranded nucleic acids (intermediate temperature zones). The temperatures within these zones fall within ranges well known in the art for conducting PCR reactions. See for example, Sambrook et al. (Molecular Cloning, A Laboratory Manual, 3rd edition, Cold Spring Harbor Laboratory Press, Cold Spring Harbor, N.Y., 2001).

In certain embodiments, the three temperature zones are controlled to have temperatures as follows: 95° C. (TH), 55° C. (TL), 72° C. (TM). The prepared sample droplets flow through the channel at a controlled rate. The sample droplets first pass the initial denaturation zone (TH) before thermal cycling. The initial preheat is an extended zone to ensure that nucleic acids within the sample droplet have denatured successfully before thermal cycling. The requirement for a preheat zone and the length of denaturation time required is dependent on the chemistry being used in the reaction. The samples pass into the high temperature zone, of approximately 95° C., where the sample is first separated into single stranded DNA in a process called denaturation. The sample then flows to the low temperature, of approximately 55° C., where the hybridization process takes place, during which the primers anneal to the complementary sequences of the sample. Finally, as the sample flows through the third medium temperature, of approximately 72° C., the polymerase process occurs when the primers are extended along the single strand of DNA with a thermostable enzyme.

The nucleic acids undergo the same thermal cycling and chemical reaction as the droplets pass through each thermal cycle as they flow through the channel. The total number of cycles in the device is easily altered by an extension of thermal zones. The sample undergoes the same thermal cycling and chemical reaction as it passes through N amplification cycles of the complete thermal device.

In other embodiments, the temperature zones are controlled to achieve two individual temperature zones for a PCR reaction. In certain embodiments, the two temperature zones are controlled to have temperatures as follows: 95° C. (TH) and 60° C. (TL). The sample droplet optionally flows through an initial preheat zone before entering thermal cycling. The preheat zone may be important for some chemistry for activation and also to ensure that double stranded nucleic acid in the droplets is fully denatured before the thermal cycling reaction begins. In an exemplary embodiment, the preheat dwell length results in approximately 10 minutes preheat of the droplets at the higher temperature.

The sample droplet continues into the high temperature zone, of approximately 95° C., where the sample is first separated into single stranded DNA in a process called denaturation. The sample then flows through the device to the low temperature zone, of approximately 60° C., where the hybridization process takes place, during which the primers anneal to the complementary sequences of the sample. Finally, the polymerase process occurs when the primers are extended along the single strand of DNA with a thermostable enzyme. The sample undergoes the same thermal cycling and chemical reaction as it passes through each thermal cycle of the complete device. The total number of cycles in the device is easily altered by an extension of block length and tubing.

After amplification, droplets may be flowed to a detection module for detection of amplification products. The droplets may be individually analyzed and detected using any methods known in the art, such as detecting for the presence or amount of a reporter. Generally, the detection module is in communication with one or more detection apparatuses. The detection apparatuses may be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module. Further description of detection modules and methods of detecting amplification products in droplets are shown in Link et al. (U.S. patent application numbers 2008/0014589, 2008/0003142, and 2010/0137163) and European publication number EP2047910 to Raindance Technologies Inc.

In another embodiment, examples of assays are ELISA assays (see, e.g., US Patent Publication No. 20100022414). The present invention provides another emulsion library which may comprise a plurality of aqueous droplets within an immiscible fluorocarbon oil which may comprise at least one fluorosurfactant, wherein each droplet is uniform in size and may comprise at least a first antibody, and a single element linked to at least a second antibody, wherein said first and second antibodies are different. In one example, each library element may comprise a different bead, wherein each bead is attached to a number of antibodies and the bead is encapsulated within a droplet that contains a different antibody in solution. These antibodies may then be allowed to form “ELISA sandwiches,” which may be washed and prepared for a ELISA assay. Further, these contents of the droplets may be altered to be specific for the antibody contained therein to maximize the results of the assay.

In another embodiment, single-cell assays are also contemplated as part of the present invention (see, e.g., Ryan et al., Biomicrofluidics 5, 021501 (2011) for an overview of applications of microfluidics to assay individual cells). A single-cell assay may be contemplated as an experiment that quantifies a function or property of an individual cell when the interactions of that cell with its environment may be controlled precisely or may be isolated from the function or property under examination. The research and development of single-cell assays is largely predicated on the notion that genetic variation causes disease and that small subpopulations of cells represent the origin of the disease. Methods of assaying compounds secreted from cells, subcellular components, cell-cell or cell-drug interactions as well as methods of patterning individual cells are also contemplated within the present invention

In other embodiments, chemical prototyping and synthetic chemical reactions are also contemplated within the methods of the invention.

In certain embodiments, nucleic acids are labeled with a nucleoside analogue. The nucleoside analogue may be any nucleoside analogue known in the art or developed after the filing of the present invention that is incorporated into replicated DNA and can be detectable by a label. The label may be incorporated into the nucleoside analogue or may include a labeling step after incorporation into DNA with a detectable label. In preferred embodiments, the label is a fluorescent label. In certain embodiments, the nucleoside analogue may be EdU (5-ethynyl-2′-deoxyuridine) or BrdU (5-bromo-2′-deoxyuridine).

In one embodiment of the invention, the method comprises obtaining at least one section from one or more tissue samples. Any suitable tissue sample can be used in the methods described herein. For example, the tissue can be epithelium, muscle, organ tissue, nerve tissue, tumor tissue, and combinations thereof. Samples of tissue can be obtained by any standard means (e.g., biopsy, core puncture, dissection, and the like, as will be appreciated by a person of skill in the art). At least one section may be labeled with a histological stain, to produce a histologically stained section. As used in the invention described herein, histological stains can be any standard stain as appreciated in the art, including but not limited to, alcian blue, Fuchsin, haematoxylin and eosin (H&E), Masson trichrome, toluidine blue, Wright's/Giemsa stain, and combinations thereof. As will be appreciated by a person of skill in the art, traditional histological stains are not fluorescent. At least one other section may be labeled with at least one fluorescently labeled reagent to produce a fluorescently labeled section. As used in the invention described herein, the panel of fluorescently labeled reagents comprises a number of reagents, such as fluorescently labeled antibodies, fluorescently labeled peptides, fluorescently labeled polypeptides, fluorescently labeled aptamers, fluorescently labeled oligonucleotides (e.g. nucleic acid probes, DNA, RNA, cDNA, PNA, and the like), fluorescently labeled chemicals and fluorescent chemicals (e.g., Hoechst 33342, propidium iodide, Draq-5, Nile Red, fluorescently labeled phalloidin), and combinations thereof. Each fluorescently labeled reagent is specific for at least one biomarker. As used herein, a “biomarker” is a molecule which provides a measure of cellular and/or tissue function. For example, and without limitation, a biomarker can be the measure of receptor expression levels, (e.g., estrogen receptor expression levels, Her2/neu expression); transcription factor activation; location or amount or activity of a protein, polynucleotide, organelle, and the like; the phosphorylation status of a protein, etc. In one embodiment, a biomarker is a nucleic acid (e.g., DNA, RNA, including micro RNAs, snRNAs, mRNA, rRNA, etc.), a receptor, a cell membrane antigen, an intracellular antigen, and extracellular antigen, a signaling molecule, a protein, and the like. In one embodiment of the invention, a panel of fluorescently labeled reagents detects at least about four different biomarkers. In another embodiment of the invention, a panel of fluorescently labeled reagents detects at least about four to about six, to about ten, to about twelve different biomarkers or more. In a further embodiment, each fluorescently labeled reagent has different fluorescent properties, which are sufficient to distinguish the different fluorescently labeled reagents in the panel.

A single biomarker can provide a read-out of more than one feature. For example, Hoechst dye detects DNA, which is an example of a biomarker. A number of features can be identified by the Hoechst dye in the tissue sample such as nucleus size, cell cycle stage, number of nuclei, presence of apoptotic nuclei, etc. In one embodiment of the invention, the imaging procedures are automated.

In one embodiment of the invention, the one or more tissue samples are isolated from one or more animals. For example, in one embodiment, the one or more animals are one or more rodents, preferably a mouse. The tissue may be isolated from a human subject. In certain embodiments tissues are isolated post mortem. In a particular embodiment, one or more tissue samples are isolated from an animal at one or more time points.

Methods of dissecting tissues from any organism are well known in the art. One method that may be utilized according to the present invention may be microdissection. Laser Capture Microdissection (LCM) enables separation of clusters of cells or even individual cells of interest from a background of millions of other cells. The collected cells can be directly visualized to verify their identity and purity. LCM is used to select small clusters of cells of interest from frozen sections of tissue by embedding them in a transfer film, e.g., a thermoplastic polymer. An example of a suitable thermoplastic polymer is ethylene vinyl acetate (EVA). The general methods of LCM are well known. See, e.g., U.S. Pat. Nos. 5,985,085; 5,859,699; and 5,843,657; as well as Suarez-Quian et al., “Laser Capture Microdissection of Single Cells from Complex Tissues,” BioTechniques, Vol. 26, pages 328-335 (1999); Simone et al., “Laser-capture microdissection: opening the microscopic frontier to molecular analysis,” TIG, Vol. 14, pages 272-276 (1998); and Bonner et al., “Laser Capture Microdissection: Molecular Analysis of Tissue,” Science, Vol. 278, pages 1481-1483 (1997).

LCM is a process by which cells and portions of biological tissue samples are acquired directly from tissue sections mounted on glass slides or other solid surfaces. Once the cells or tissue portions of interest (tissue targets) are located in the sample, a laser is focused over the tissue targets. When the laser is fired, the thin-film located directly above the tissue targets melts, flows down and adheres to the tissue targets. The tissue targets are now stabilized and ready for molecular analysis.

The present may also be performed on tissue samples isolated from transgenic animals, such as mice. In certain embodiments, the animals may express a transgene. The transgene may be expressed in a specific cell type (e.g., a neuron). Expression of the transgene may produce a marker that can be used to enrich for single cells or nuclei of a specific cell type. In certain embodiments, the animal may express a genome editing system such as described in “In vivo interrogation of gene function in the mammalian brain using CRISPR-Cas9” Swiech L., et al., Nat Biotechnol October 19. (2014). The animal may be xenograft. Xenotransplantation of tumor cells into immunocompromised mice is a research technique frequently used in pre-clinical oncology research. The tissue may express a transgene for isolating tissue specifically from a tumor. The tissue may be labeled with a nucleoside analogue in order to isolate cells of a developmental stage.

In some embodiments, the method further comprises filtering the single nuclei, as described elsewhere herein. In some embodiments, nuclei doublets are removed by filtering.

In some embodiments, nuclei containing ambient RNA or ambient RNA alone are removed by filtering.

The invention is further described in the following examples, which do not limit the scope of the invention described in the claims.

### EXAMPLES

### Example 1

### Performing Single Cell Genomics in FFPE Tissue

### Summary of Results

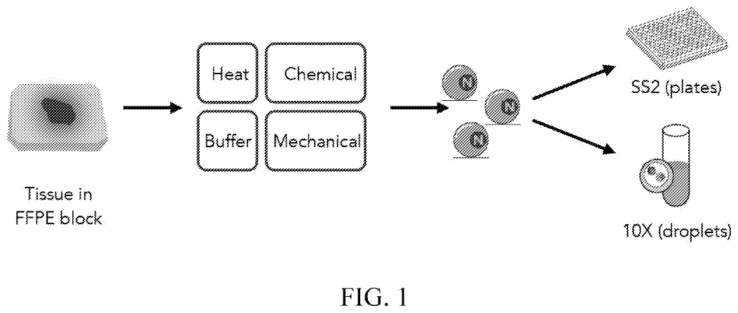

Extracting single nuclei or cells from FFPE samples requires many variables, including temperature, chemical, buffers, and mechanical variables (FIG. 1). cDNA may be obtained from single nuclei by sorting the nuclei into plates or droplets (FIG. 1). Applicants varied extraction methods and were able to isolate nuclei and whole cells from FFPE. Nuclei and whole cells can subsequently be used for transcriptome analysis; RNA extraction, cDNA generation, WTA amplification (whole transcriptome amplification), library construction, sequencing, and cell type identification. Nuclei and whole cells can subsequently be used for chromatic analysis using single cell/nucleus ATAC-seq, single cell/nucleus ChIP, or bulk (pooled) nuclei analysis using these methods. Single cells/nuclei can subsequently be used for single cell/nucleus DNA sequencing (e.g. cancer mutations in single cells). Single cells and nuclei can be stained by antibodies and FACS sorted following isolation from FFPE to isolate specific single cells or to get single-cell type population profiling for transcriptomes, DNA sequences (e.g. mutations in cancer), or epigenomic analysis. Applicants are developing low-RNA input transcriptome generation. This has been done down to 33 μg. Applicants can perform RNA analysis from bulk FFPE extracted nuclei. Applicants have obtained WTA from 5000 pooled nuclei as assessed by a bioanalyzer. Determinants of RNA quality from FFPE samples has been described previously (see, e.g., von Ahlfen et al., 2007, Determinants of RNA Quality from FFPE Samples. PLoS ONE 2(12): e1261).

### Tissue Extraction and Nuclei Isolation Method 1:

Cut excess paraffin from tissue (FFPE brain) and split into 30-100 mg pieces
Dissolve paraffin at room temperature with two×10-minute changes of xylene (5 mL each)
Perform 1 wash at 37 C for 10 min
Cut tissue into smaller pieces, take 1 piece/tube and repeat 37 C wash.
The tissue was then rehydrated with 100 μl of 95%, 75%, and 50% ethanol (EtOH) for 2 minutes each
The tissue was either chopped in CST or TST for 10 min or dounce homogenized. (these are buffers from the Raisin-seq filing).
Tissue was filtered through 40 uM filter
Tissue was washed in ST and filtered again in 30 uM filter
Images taken and FACS test with Ruby stain

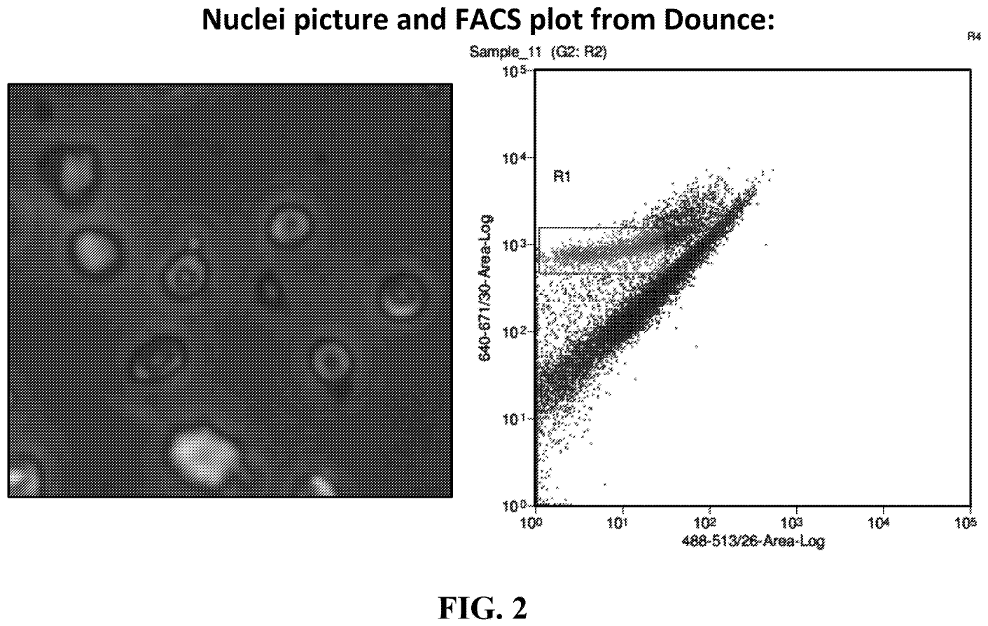

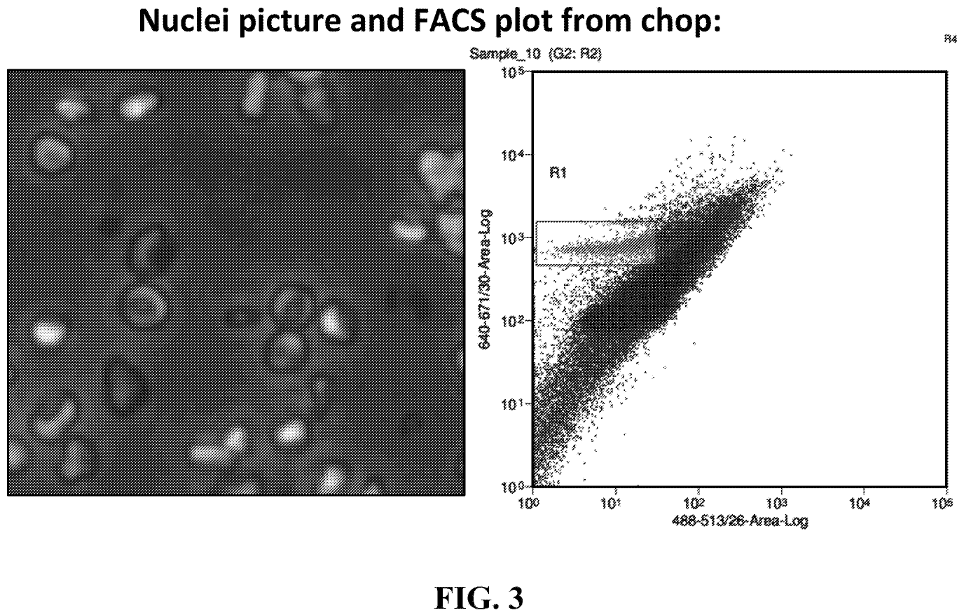

Results are shown using dounce homogenization (FIG. 2) and chopping (FIG. 3).

### Tissue Extraction and Nuclei Isolation Method 2:

Cut tissue (FFPE brain) out of paraffin
Dissolve paraffin:
   Room temperature with three 10-minute changes of xylene (1 mL each) in the microcentrifuge tube
   Room temperature for 10 min and then 2×90 C, 10 min washes
For each change, remove xylene
The tissue was then rehydrated with 100 μl of 95%, 75%, and 50% ethanol (EtOH) for 2 minutes each.
Split each tissue in ½ and re-suspend in NST
Dounce and either add PK (proteinase K) or proceed to spin without PK.
For PK, add PK to ST and proceed
Enzymatic digestion was then performed by adding 100 μl of freshly prepared proteinase K solution. Stock at 800 U/ml, use at 1:50 so for 1 mL add 20 uL and incubate at RT for 10 minutes.
Spin down and re-suspend in ST
Ruby stain, sort and also analyze by microscope

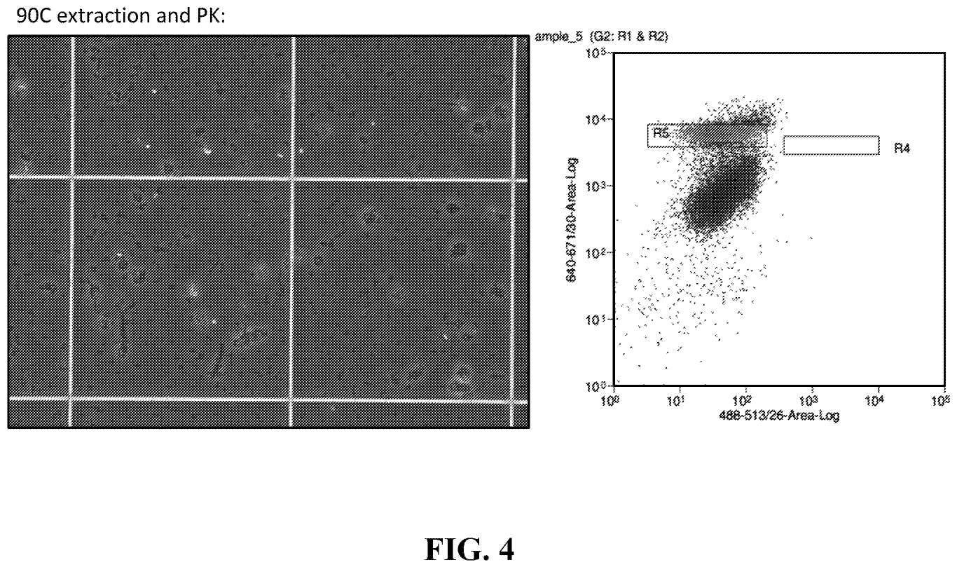

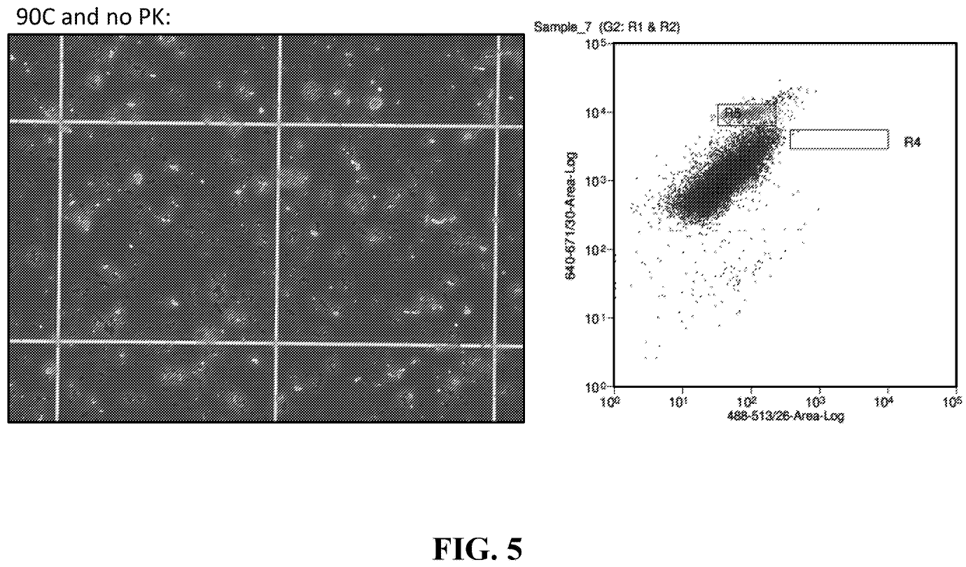

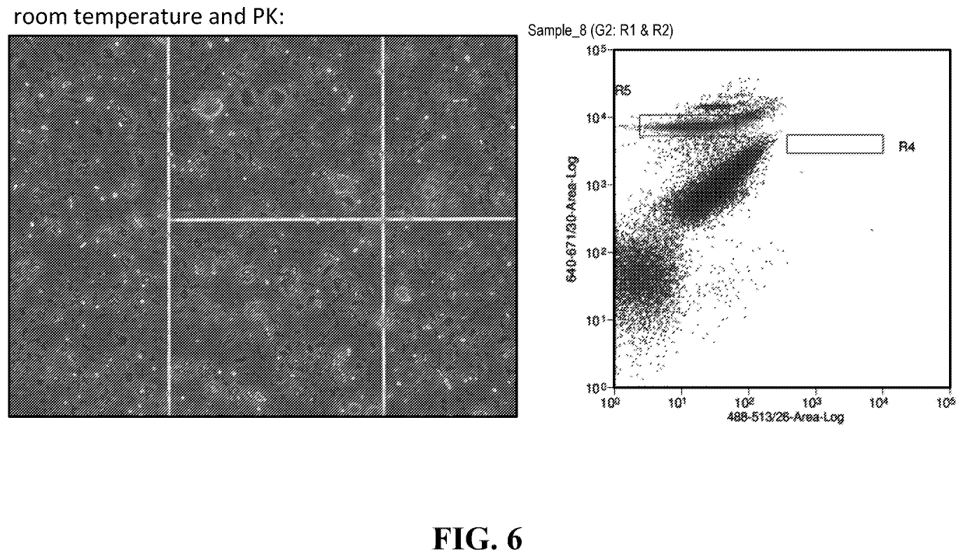

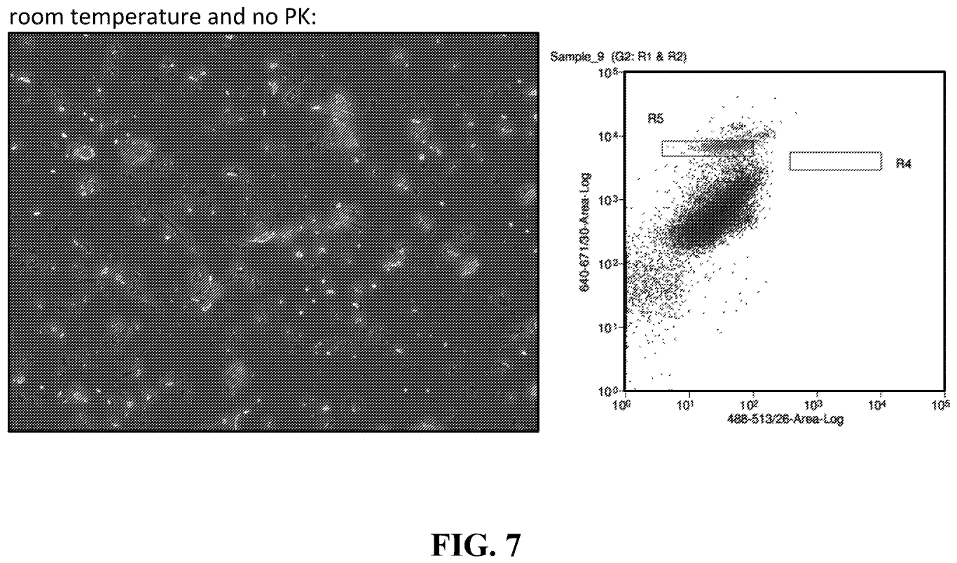

Results of method 2 are shown in FIGS. 4-7.

### Tissue Extraction and Nuclei Isolation Method 3:

Nuclei and whole cells are isolated depending on temperature (e.g., 90 C steps for nuclei and room temperature steps for cells).
Add protease inhibitors to CST and ST buffers prior to starting
Cut tissue out of paraffin (B16 and D4M.3A FFPE tumor tissue; melanoma PDX)
Dissolve paraffin in lml xylene at RT for 10 min
Divide tissue in half:half. Tissue will get two additional 10 min washes in lml xylene: either at room temperature or at 90 C.
Rehydrate tissue with 1 mL of 95%, 75%, and 50% ethanol (EtOH) for 2 minutes each.
All subsequent steps on ice.
Place tissue into 1 mL of CST and chop for 10 min
Bring to 2 mL with CST
Filter in large 40 uM filter
Add 3 mL of ST
Spin down at 500 g for 5 min and re-suspend in 500 uL ST
Examine under microscope

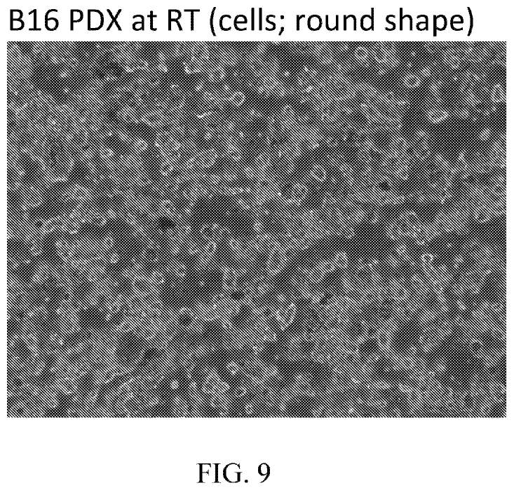

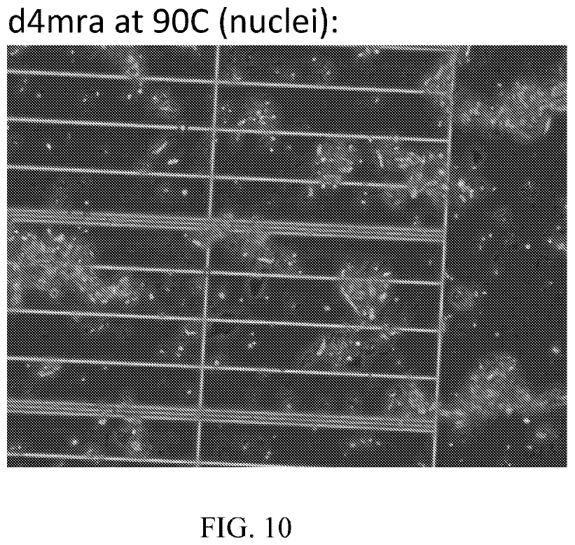

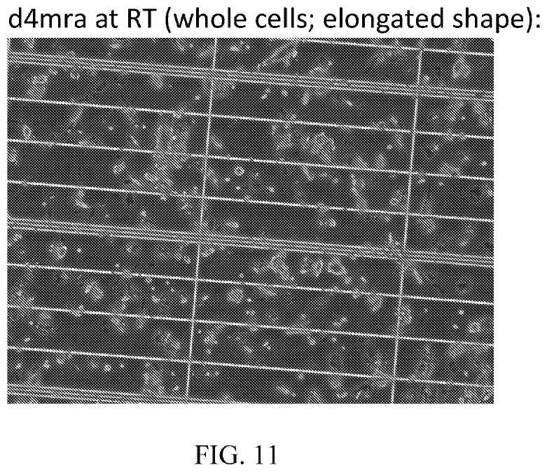

Results of method 3 are shown in FIGS. 8-11.

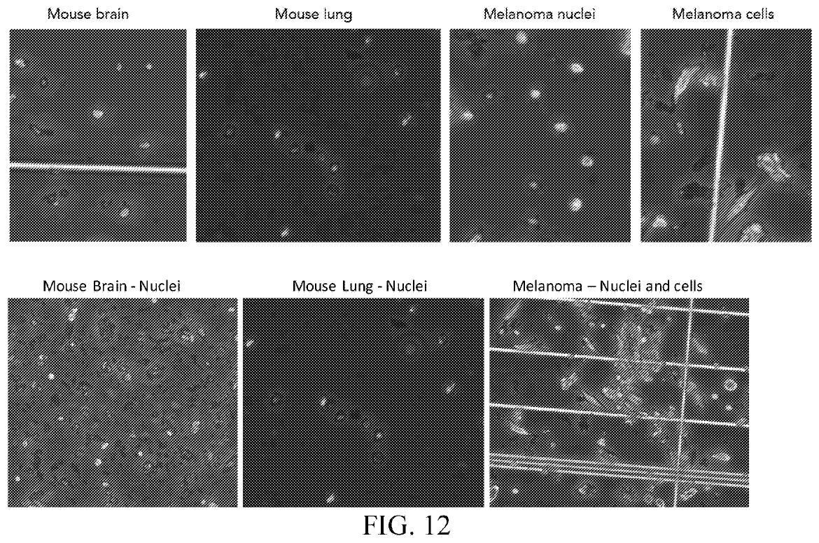

Applicants have tested several protocols for nuclei extraction (FIG. 12). These are examples of what the nuclei suspensions look like with filtering alone for debris removal. The mouse brain nuclei image was from an experiment that tested use of heat and/or proteinase K on deparaffinization using NST buffer. The Melanoma Nuclei and cells image was taken from an experiment omitting heat from the deparaffinization step, and chopping in CST buffer. The Mouse Lung nuclei image was from an experiment that tested using Mineral Oil and heat deparaffinization, and douncing or chopping. These are representative images showing that the methods yield nuclei. Additional images of nuclei and cell extraction are also shown.

### Example 2

### FFPE RNA Extraction and Whole Transcriptome Amplification (WTA)

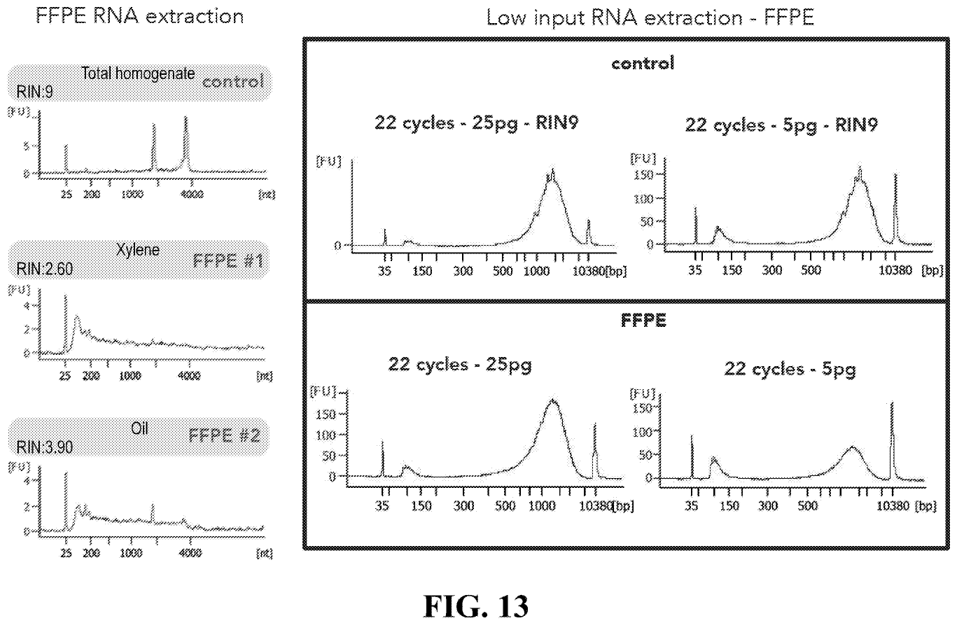

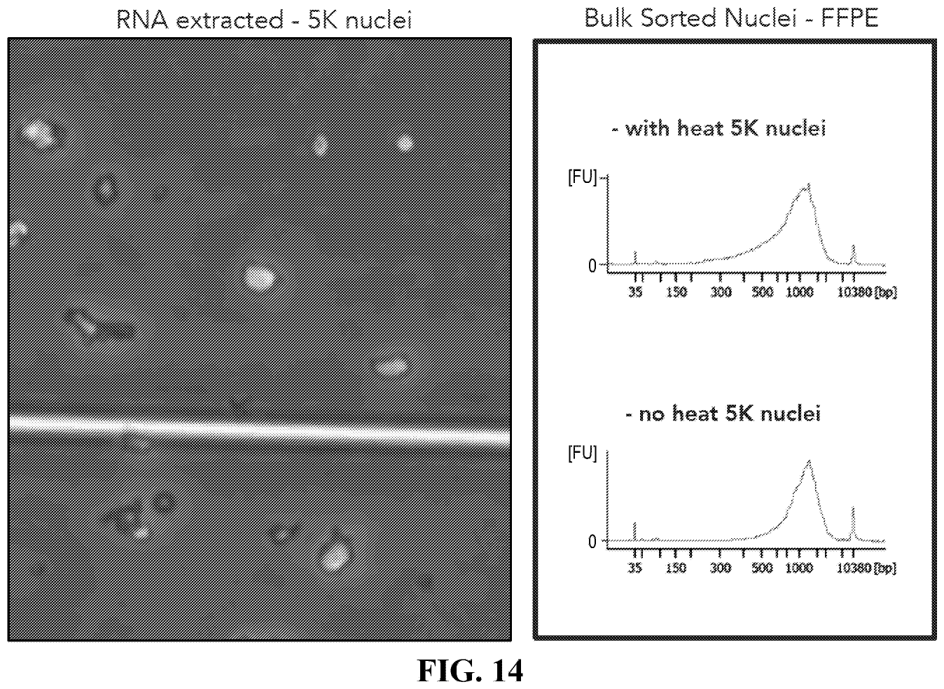

Applicants performed RNA extraction of FFPE tissue using FormaPure RNA extraction kit. This kit uses mineral oil for deparaffinization. Applicants also modified the beginning of this protocol to use Xylene for deparaffinization. The RNA quality was low in the Xylene and oil experiments compared to the control (FIG. 13). The control was frozen tissue extracted using Qiagen RNeasy kit with DNA eliminator columns. The FormaPure FFPE RNA extraction kit most similarly follows the SMART-Seq2 protocol in that it also uses SPRI beads for total nucleotide extraction. There is an option to elute with a DNAse I digestion and rebind the RNA to the SPRI beads. Applicants did not perform that step as it is not used for the SS2 protocol. RNA was quantified by Qubit RiboGreen HS RNA kit, which only binds to RNA and not double-stranded DNA. Applicants analyzed cDNA production with the low input RNA extraction from FFPE. Applicants observed high quality cDNA traces from FFPE bulk extractions (FIG. 13). Low input yields could be improved with added PCR cycles. Applicants extracted RNA from 5000 nuclei and tested cDNA from RNA extracted from bulk sorted FFPE nuclei with and without heat (FIG. 14). Applicants observed high quality cDNA under both conditions.

Applicants extracted RNA from FFPE of mouse brain tissue using this kit: FormaPure RNA cat. no. C19683AB with the following modifications to the manufacturer protocol

### Deparaffinization by Xylene

Cut a tiny section of tissue from the FFPE block.
in 1.5 mL tubes, dissolve paraffin in Xylene:
   Room Temp. for 10 mins and then 2×90 C, lmL each wash
   For each change, remove xylene
Rehydrate with 1 mL of 90% Ethanol, then 75%, then 50% for 2 mins each at room temp.
Rinse with ice cold ST buffer to remove last traces of ethanol.
Proceed to FormaPure protocol step: 3 Tissue Digestion
Note: will not observe a phase separation
   Skip step 4—no need to remove lower phase to a new tube.
   Make careful observations of how well the tissue is dissolved. (can include a homogenization step)
Proceed to step 5 with no other modifications to the protocol

### Deparaffinization by FormaPure Method (Mineral Oil)

Transfer 310 um thick sections of tissue to a 1.5 mL tube and add 450 ul of Mineral Oil.
   Note: FFPE blocks are not prepared properly to use a microtome. The tissue can be minced prior to adding to mineral oil.
Follow FormaPure protocol and make careful observations of how well paraffin is dissolved and tissue is lysed.

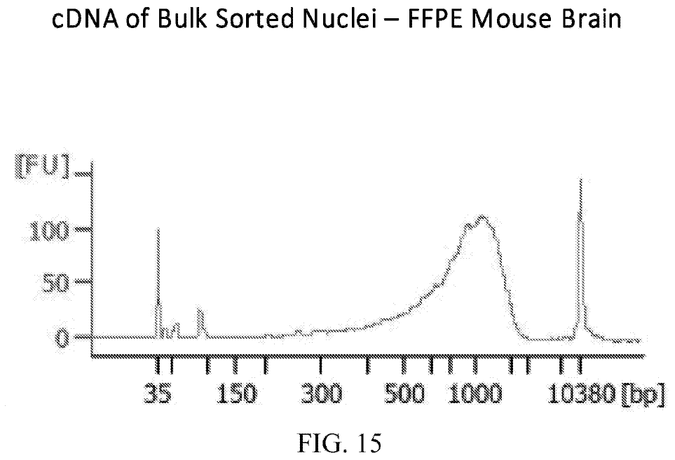

SS2 of bulk sorted nuclei without modifications does not yield any measurable amount of cDNA. Adding a Proteinase K heat step to help reverse cross linking of sorted and lysed nuclei works well (FIG. 15) (5,000 nuclei are sorted into 5 ul of TCL+1% BME lysis buffer−Final volumes are around 15-17 ul. Removed 15 ul to a new plate for SS2). cDNA traces are still of high quality with large fragment sizes. (5000 nuclei and 14 cycles of PCR). Applicants can perform library construction and sequencing. Applicants also tested including after the Proteinase K digestion, an extra heat step which acts to reverse cross link RNA and also to inactivate the Proteinase K. These samples need SPRI cleaning and this extra heat step does seem to cause some degradation—although yields may be slightly increased.

Following the sNuc-Seq SMART-Seq2 protocol with a range of input concentrations of RNA Applicants added 1 ul of RNA to 4 ul of the Mix 1 and proceeded from step 22.

Input RNA concentrations across 12 wells in rows (Table 4):

TABLE 4
Using 1 ul added to 4 ul of Mix 1

| ng/ul | pg/ul |
|---|---|
| 0.5000 | 500.0 |
| 0.2500 | 250.0 |
| 0.1250 | 125.0 |
| 0.0625 | 62.5 |
| 0.0313 | 31.3 |
| 0.0156 | 15.6 |
| 0.0078 | 7.8 |
| 0.0039 | 3.9 |
| 0.0020 | 2.0 |
| 0.0010 | 1.0 |
| 0.0005 | 0.5 |
| 0.0000 | 0 |

TABLE 5
Qubit Results:

| | | | | Frozen |
|---|---|---|---|---|
| | pg | Xylene | Mineral Oil | (high RIN control) |
| Well | input | Row B | Row C | Row D |
| 1 | 500.0 | 4.53 | 7.64 | 37.8 |
| 2 | 250.0 | 4.15 | 5.02 | 23.2 |
| 3 | 125.0 | 2.92 | 3.03 | 10.4 |
| 4 | 62.5 | 1.98 | 2.14 | 8.14 |
| 5 | 31.3 | 1.49 | 1.70 | 2.76 |
| 6 | 15.6 | 0.969 | 0.965 | 1.13 |
| 7 | 7.8 | 1.25 | 0.841 | 0.861 |
| 8 | 3.9 | 1.48 | 0.934 | 0.802 |
| 9 | 2.0 | 0.761 | 0.811 | 0.530 |
| 10 | 1.0 | 1.04 | 1.06 | 0.642 |
| 11 | 0.5 | 1.38 | 1.20 | 1.07 |
| 12 | 0 | 1.20 | 0.710 | 0.756 |

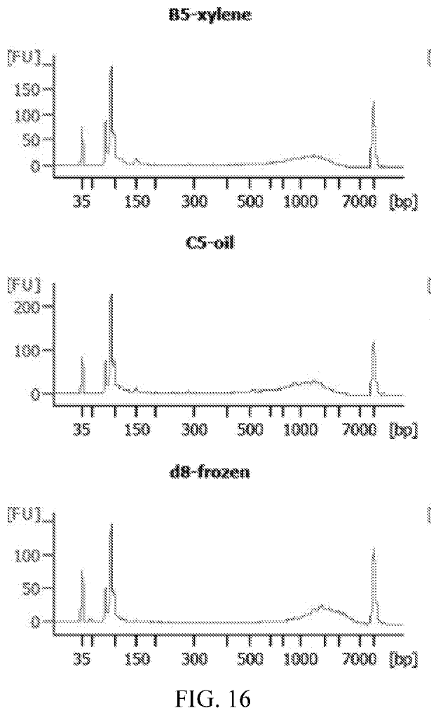

Highlighted wells were also run on BioAnalyzer High Sensitivity Chip (FIG. 16).

WTA Preparation from FFPE Extracted Nuclei:

### Xylene Deparaffinization:

1. Using a 1.5 mm punch biopsy tool to section tissue from FFPE blocks
2. Add 1 mL xylene to tissue in eppendorf tubes—in fume hood.
3. Incubate 10 min at RT, and then 2×90 C, 1 mL each wash. For each change, remove xylene, and wrap caps with parafilm
4. Rehydrate tissue with 1 mL of 95%, 75% and 50% ethanol for 2 mins each at room temp.
5. All subsequent steps on ice, move quickly
6. Place tissue in 1 mL of CST for chop in a well of 6 w plate, chop for 10 mins.
7. Add 1 mL of CST and filter
8. Raise volume to 5 mL with ST buffer—5 mL final volume
9. Centrifuge at 500 g for 5 mins (lower brake speed to 5)
10. Remove supernatant, and resuspend pellet in desired volume of ST buffer plus 0.04% BSA
11. examine under microscope, and count with cellometer.

### Mineral Oil Deparaffinization

1. Add 450 ul of mineral oil to tissue in eppendorf tubes, incubate at 80 C for 15 mins.
2. Remove mineral oil and Rehydrate with 1 mL of 95%, 75% and 50% ethanol for 2 mins each at room temp.
3. Continue from step 4 above.

Add Ruby to each sample and sort with the SONY sorter (FACS).

### Prepare Lysis Plates for Sorting

6 Plates each of TCL+BME, and 4 Plates of TritonX-100 using Eppendorf twin.tec PCR Plate 96, skirted, colorless

Make 750 ul of each lysis buffer:

TCL buffer—add 10 ul per mL for 1% solution

TABLE 6

| Reagent | 1 rnx | 750 ul | Final Conc | |
|---|---|---|---|---|
| TritonX-100 (10%) | 0.08 | 15 | 0.2% | |
| Trehalose (1M) | 3.6 | 697.5 | 0.93 | M |
| RNase Inhibitor (40 U/ul) | 0.2 | 37.5 | 2 U/ul | |

a. Aliquot 85 ul to 8 wells of strip tube and use a multichannel to pipet 5 ul of TCL+1% BME to each well of columns 1 and 2 of 6 plates

b. Aliquot 85 ul to 8 wells of a strip tube and use a multichannel to pipet 4 ul of TritonX-100 lysis buffer to each well of columns 1 and 2 of 6 plates

Seal plates and place one ice. Prior to sorting, spin them down.

### SS2 of Bulk Samples:

Using one sample of bulk—A1 in TCL+1% BME. Add wells of RNA at 1 ng and 5 ng total input, and use 14 cycles of amplification for cDNA Amp. Also include an no template control (NTC) for 4 wells total. Take total RNA with RIN 8 or better, dilute to 1 ng/ul and 0.2 ng/ul for the 1 ng and 5 ng input positive controls.

5,000 nuclei (measured volume to be around 15-17 ul); added 34 ul of SPRI
5 ng RIN 9-10 ul of each (5 ul of TCL buffer, plus the 5 ul of RNA controls)
1 ng RIN 9
5 ng Xylene RNA
NTC

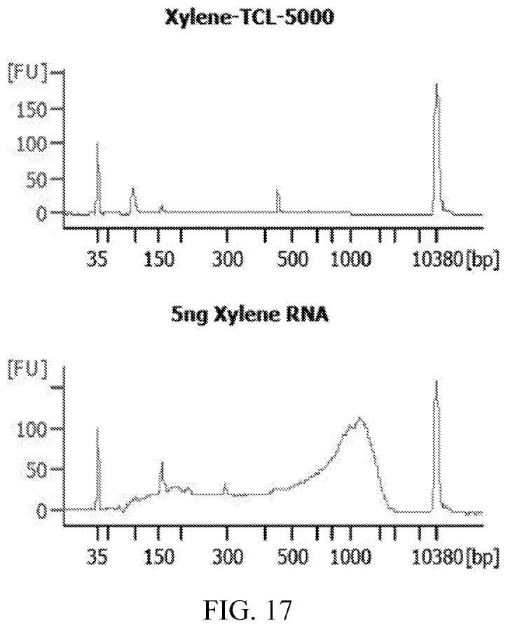

Applicants used 34 ul of SPRI for all of these and proceeded with the protocol eluting in 4 ul of Mix 1. Applicants observed that the nuclei did not amplify as the RNA controls did (FIG. 17). Applicants hypothesized that cross-linking was not fully reversed.

### Test Using Proteinase K

Prior to SPRI nucleotide purification from lysate, pick a bulk lysate from the TCL and the Triton X-100 lysis buffers, and include 1 ng RNAs as controls—degraded xylene extracted RNA, and RIN9 and NTC. Take 15 ul of the bulk sorted nuclei—(the volume from the sorter significantly raises the volume of the sample). all of it.

Make a Proteinase K dilution and add 1 ul to each sample:
NEB P8107S 800 U/mL=20 mg/mL=20 ug/ul=0.8 U/ul
Use 1 ul and dilute into 49 ul of water
Set Thermal Cycler to 60 C for 60 mins and on for 55 C for 15 mins
Samples for 60 C for 60 mins
A—5K nuclei—TX lysis buf—mineral oil isolation (take 15 ul)
B—5K nuclei—TCL lysis buf—mineral oil isolation (take 15 ul)
C—1 ng RIN 9 positive control
D—1 ng Xylene extracted total RNA (RIN 2)
E—NTC
F—5K nuclei—TX lysis buf—xylene isolation
Samples for 55 C for 15 mins
A—5K nuclei—TX lysis buf—mineral oil isolation
B—5K nuclei—TCL lysis buf—mineral oil isolation
C—1 ng RIN 9 positive control
D—1 ng Xylene extracted total RNA (RIN 2)
E—NTC

Applicants used maxima RT enzyme and 14 cycles of PCR.

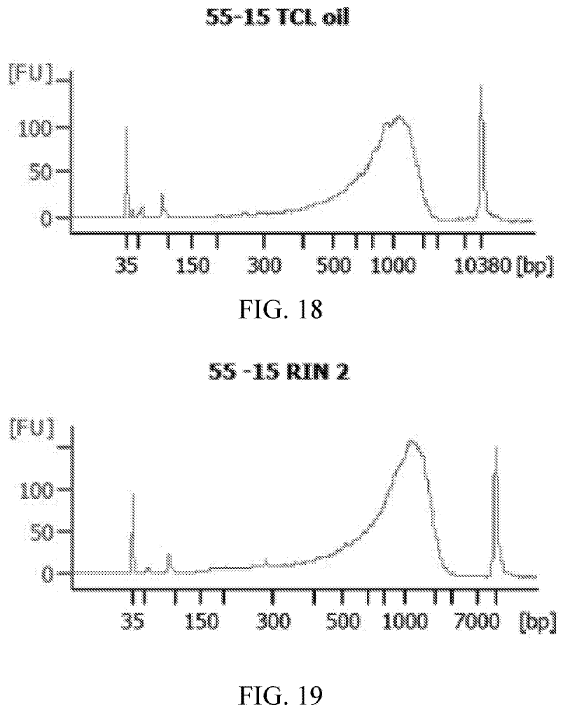

Applicants observed that the TX lysis buffer does not work as the nuclei probably did not lyse. The 55 for 15 min plate obtained good WTA from the bulk nuclei in TCL buffer (FIG. 18). The 55 for 15 min plate obtained good WTA from the Xylene extracted total RNA (FIG. 19).

### Example 3

### FFPE Materials and Methods

TCL lysis buffer (Qiagen, #1031576) was used as described herein. Single nucleus RNA was first purified using RNAClean XP beads (Beckman Coulter, Agencourt RNA-Clean XP, #A63987) at 2.2× beads to sample volume ratio. Single nucleus derived cDNA libraries can be generated following a modified Smart-seq2 method. Reverse transcription (RT) can be performed with Maxima RNase-minus RT (Thermo Fisher Scientific, Maxima Reverse Transcriptase, #EP0752), 2 μl 5× Maxima RT buffer, 2 μl Betaine (Sigma Aldrich, 5M, #B0300), 0.9 μl MgCl2 (Sigma Aldrich, 100 mM, #M1028), 1 μl TSO primer (10 μm), 0.25 μl RNase inhibitor (40 U/μl). Samples can then be amplified with KAPA HiFi HotStart ReadyMix (KAPA Biosystems, #KK2602). PCR product can be purified using AMPure XP (Beckman Coulter, Agencourt AMPure XP, #A63880) and eluted in TE buffer (Thermo Fisher Scientific, #AM9849). Purified cDNA libraries can be analyzed on Agilent 2100 Bioanalyzer (Agilent, Agilent High Sensitivity DNA Kit, #5067-4626) and quantified using picogreen (Thermo Fisher Scientific, Quant-iT PicoGreen dsDNA Assay Kit, #P11496) on a plate reader (Biotek, Synergy H4, wavelength at 485 nm, 528 nm with 20 nm bandwidth). Sequencing libraries can be prepared using Nextera XT kit (Illumina, #FC-131-1024) as described previously. Chopping can use sharp dissection scissors for 10 min. 40 micron nylon cell strainer (Falcon 352340) may be used.

Pieces of tissue should be small; less than 30, 40, 50, 60, 70, 80, 90, 100 or 200 mg, or less than about 1 cm3, or half an almond. If tissue is limited, one can go as low as 10, 15, 20 or 25 mg for a single preparation.

In certain embodiments, buffers were used to extract nuclei by chopping tissue with scissors for 10 minutes in the respective buffer. In certain embodiments, extracted nuclei or cells were filtered through a 40 micron filter, and washed once. Compositions of buffers used are shown in Table 7 and Table 8. Reagents used to make buffers were procured from VWR, Sigma, and other vendors. Alternative buffer component concentrations that deviate from the buffers below may be used. In certain embodiments, tricine may improve small molecule diffusion. Regarding buffering agents (e.g., Tris, Tricine, HEPES, PIPES) if a tissue is neutral pH then the buffer concentration may be close to zero (e.g. 1 mM). Regarding detergents, Applicants tested down to 0.0012 for tween-20. In certain embodiments, the concentration for detergents is between 0.001 or 0.0005%. In certain embodiments, detergent concentration is up to 1-2%. Regarding salts, the buffer may be adjusted down to 10 mM for NaCl, 0.1 mM for CaCl2, and 1 mM for MgCl2. Regarding polyamines, the buffer may be adjusted down to 0.1 mM for both spermidine and spermine.

TABLE 7
Compositions of Buffers

| | Buffer | | Deter- | Salt and | Additives |
|---|---|---|---|---|---|
| | Concen- | Deter- | gent Concen- | Concen- | and Concen- |
| Buffer | tration | gent | tration (%) | tration | tration |
| Tris | 10 mM | NP40 | 0.2 | 146 mM NaCl, | |
| | | | | 1 mM CaCl₂, | |
| | | | | 21 mM MgCl₂ | |
| Tris | 10 mM | CHAPS | 0.49 | 146 mM NaCl, | |
| | | | | 1 mM CaCl₂, | |
| | | | | 21 mM MgCl₂ | |
| Tris | 10 mM | Tween- | 0.03 | 146 mM NaCl, | |
| | | 20 | | 1 mM CaCl₂, | |
| | | | | 21 mM MgCl₂ | |
| Tricine | 20 mM | NP40 | 0.2 | 146 mM NaCl, | 0.15 mM |
| | | | | 1 mM CaCl₂, | spermine |
| | | | | 21 mM MgCl₂ | and 0.5 mM |
| | | | | | spermidine |

TABLE 8
Compositions of Buffers.

| | | | | Detergent | | |
|---|---|---|---|---|---|---|
| | | Buffer | | concentration | | Additives |
| Composition | Buffer | conc. | Detergent | (%) | Salt conc. | concentration |
| ST | Tris | 10 mM | | | 146 mM NaCl, 1 mM | |
| | | | | | CaCl2, 21 mM MgCl2 | |
| CST | Tris | 10 mM | CHAPS | 0.49 | 146 mM NaCl, 1 mM | 0.01% BSA |
| | | | | | CaCl2, 21 mM MgCl2 | |
| TST | Tris | 10 mM | Tween-20 | 0.03 | 146 mM NaCl, 1 mM | 0.01% BSA |
| | | | | | CaCl2, 21 mM MgCl2 | |
| NSTnPo | Tricine | 20 mM | NP40 | 0.2 | 146 mM NaCl, 1 mM | 0.15 mM |
| | | | | | CaCl2, 21 mM MgCl2 | spermine |
| | | | | | | 0.5 mM |
| | | | | | | spermidine |
| | | | | | | 0.01% BSA |
| NST | Tris | 10 mM | NP40 | 0.2 | 146 mM NaCl, 1 mM | 0.01% BSA |
| | | | | | CaCl2, 21 mM MgCl2 | |

### Example 4

### sNucER-seq

Previously, Applicants developed single nucleus RNA sequencing (sNuc-seq) as a method to profile the expression of single cells. The outer membrane of the nucleus is continuous with the rough endoplasmic reticulum (RER). The RER is a site of RNA translation. Preserving a portion of it with the nucleus would improve RNA recovery and single cell expression profiling. Applicants conducted a screen to improve sNuc-seq. The compositions of nuclei isolation solutions that worked best preserve a portion of the nuclear outer membrane/RER along with ribosomes as determined by electron microscopy. This method is referred to as single nucleus and rough endoplasmic reticulum (sNucER)-seq.

Screen summary: Applicants focused on the enteric nervous system, which represents a rare cell population in a complex tissue. Applicants used a double transgenic mouse which labels enteric nervous system nuclei with GFP and allows for FACS following nuclei isolation. Selected nuclei were processed using smart-seq2 and sequenced.

Detergents: Applicants conducted a screen to optimize single nucleus RNA profiling of cells from tissues. Applicants tested a range of detergents that have previously been reported for nuclei extraction (Tween-20, Nonidet P-40/IGEPAL CA-630, Digitonin), and not reported (CHAPS). Applicants also compared a commercial nuclei extraction reagent (Nuclei EZ lysis buffer, SIGMA).

Based on the published literature it was not clear which concentrations of detergents would be optimal for nuclei extraction for sNuc-seq. Additionally, there was no data on CHAPS. Applicants chose to include CHAPS to increase detergent diversity. Tween-20, and Nonidet P-40/IGEPAL CA-630 are both non-ionic detergents. CHAPS is a zwitterionic detergent; as a note, CHAPS performed the best, and it is likely other zwitterionic detergents could do equally well.

Applicants chose the detergent concentrations based on the critical micelle concentration (CMC) for each detergent. Applicants then varied it either above or below the CMC.

Buffers: As part of the screen, Applicants also tested different buffers that have been used in the literature (Tris, Tricine, and HEPES). Although Tris performed the best, it is likely that the buffer choice is less critical than the detergents.

Salts: Applicants chose fixed salts concertation for the tests, although Applicants did try hypotonic solutions. The salts concentration was based on cellular concentrations of salts and what has been previously reported. Applicants used 146 mM NaCl, 1 mM CaCl2, and 21 mM MgCl2. The NaCl concertation can likely be varied up to 300 mM, or completely eliminated, and replaced with another salt such as KCl (as has been done in various biochemistry preparations as needed). Similar, CaCl2 can likely be replaced with other calcium containing salts and concentrations can be increased to 20 mM or more. The same is true for varying MgCl2 or adding in other salts.

Results: From the screen Applicants identified four compositions that worked the best for isolating enteric nervous system nuclei (appropriate cell types detected, high gene representation of expected cell types, most genes per cell, least background) (see, Table 2).

Applicants performed a further comparison among these four and compositions 2 and 3 (Table 2) performed the best. Applicants examined these nuclei preparations with electron microscopy and found that they preserved a portion of the outer nuclear envelope/RER with the nuclei. As a comparison, Applicants tested the commercial Nuclei EZ lysis buffer from Sigma, which did not preserve the nuclear envelope. Applicants are in the process of performing EM on preparations from the other 2 buffers.

CST with 0.49% CHAPS was the top extraction solution with the highest ENS score and lowest contamination. The nuclei have a nuclear membrane (not double membrane in all places), the membrane contiguous with RER and has ribosomes, and mitochondrial contamination was reduced.

Applicants found that the CST buffer has a lower intron/exon ratio compared to nuclei-only preps with EZ lysis reagent supporting more spliced RNA. The Intron/Exon ratio for each were as follows: CST=1.27904; EZ frozen=1.642955; and EZ chop=2.081659.

Additionally, Applicants confirmed that droplet based, DroNcER-seq works and that the isolated nuclei are compatible with the Chromium 10× single cell system. Additionally, Applicants are testing whether sNucER-seq works with other cell types and tissues. Preliminary data suggest the method is compatible with epithelial cells, brain cells, most cell types tested (immune, epithelial, vasculature, lyphatics, muscle, adipose, neuron, glia, muscle) and the 10× system.

### Example 5

### The Enteric Nervous System of the Human and Mouse Colon at a Single-Cell Resolution

The enteric nervous system (ENS) is an extensive network of neurons and glia along the gastrointestinal (GI) tract, which coordinates motility, digestion, nutrient absorption, and barrier defense (1). The human ENS rivals the spinal cord in complexity (2). The ENS is broadly partitioned into the myenteric (Auerbach's) plexus and submucosal (Meissner's) plexus (3); with anecdotally reported differences in anatomy and composition within ganglia, across intestinal regions, and among species (2). In addition, other factors were proposed to contribute to ENS diversity, including age (4), sex (5), circadian oscillations (6), and functional dysmotility disorders (7).

The ENS is implicated in a broad range of intra- and extra-intestinal disorders. Primary enteric neuropathies, including Hirschsprung's disease, chronic intestinal pseudo-obstruction, Waardenburg syndrome type IV, and MASH1 deficiency, directly affect enteric neurons, result in agangliosis and impaired GI transit (2) and are poorly understood (8). Moreover, studies of neuro-epithelial and neuro-immune interactions (1), such as neuronal activation of group 2 innate lymphoid cells (ILC2s) (9), suggest that ENS dysfunction can impact local inflammation, motivating ENS characterization in other diseases that affect the gut (10). Intriguingly, several extra-intestinal disorders, including those affecting the central nervous system (CNS) (e.g., autism spectrum disorders (11) and Parkinson's disease (12)) are associated with early GI motility dysfunction. However, the pathophysiology of the ENS across these disorders, including affected cell types, is poorly understood.

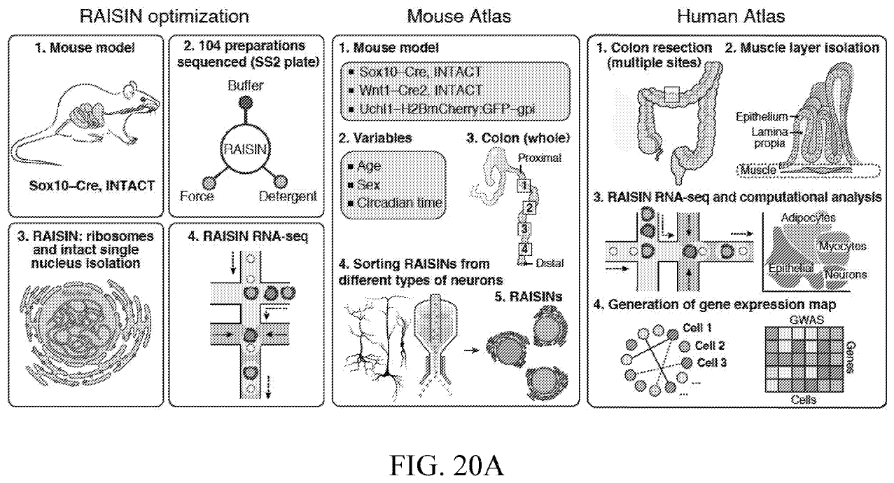

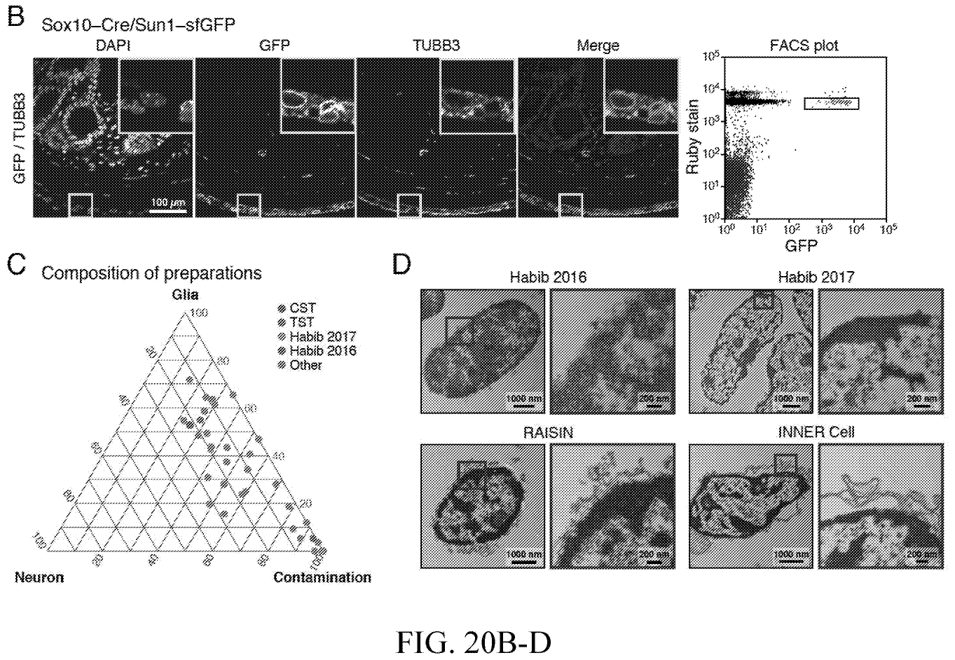

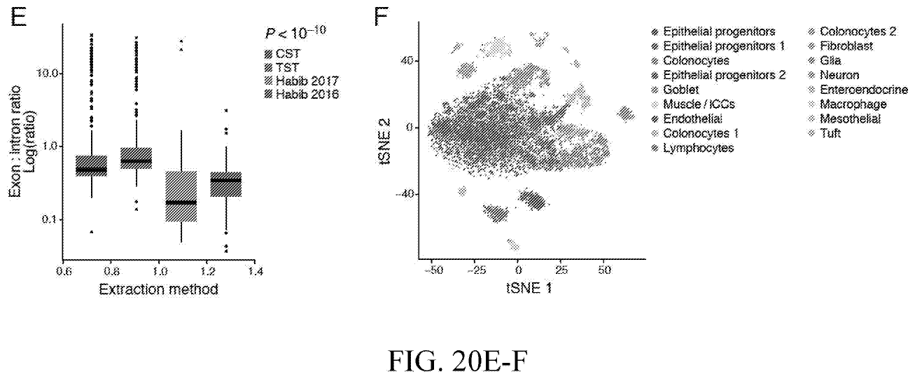

Here, Applicants generated a reference map of the ENS at single cell resolution across age, gender, location, circadian phases, and species (FIG. 20A). Applicants first developed a new method, Ribosomes And Intact SIngle Nucleus (“RAISIN”) RNA-seq, and applied it to generate a high quality single-cell census of the ENS in adult humans and mice, overcoming challenges in single-cell and single-nucleus RNA-seq (scRNA-Seq, snRNA-seq) (14-18) of the ENS. In the mouse, Applicants used genetic tools to directly enrich for and profile 2,447 enteric neurons using deep, full-length snRNA-Seq spanning four colon segments (proximal to distal) of three transgenic models (both sexes, multiple ages, two phases of the circadian rhythm). In humans, where enrichment was not possible, Applicants sequenced 163,741 single RAISINs (i.e. nuclei and attached ribosomes) from the muscularis propria of 10 individuals (men and women; 35-90 years old) and identified diverse cell types, among them 831 enteric neurons and 431 rare Interstitial Cells of Cajal (ICCs). Enteric neurons partitioned into 24 murine and 11 human subsets, which Applicants annotated with putative functions (e.g., motor, sensory, secretomotor) using known marker genes, and matched between the two species based on conserved transcriptional programs. Applicants mapped signaling interactions between human enteric neurons and other cell types in the colon, identifying possible neuro-immune, neuro-adipose, neuro-epithelial, neuro-muscular, and neuro-ICC regulatory pathways. Finally, Applicants show that enteric neurons express genes specifically associated with primary enteroneuropathies, inflammatory disorders of the gut, as well as with CNS disorders with early gut motility dysfunction, highlighting their potential roles in these disorders.

### Example 6

### Systematic Optimization of Nuclei Extraction Conditions Enables Profiling of Single ENS Nuclei from the Colon

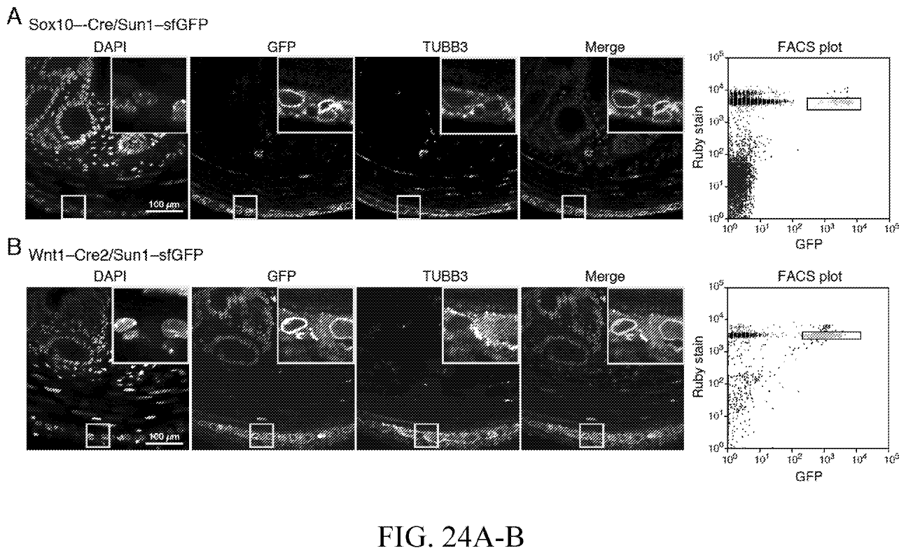

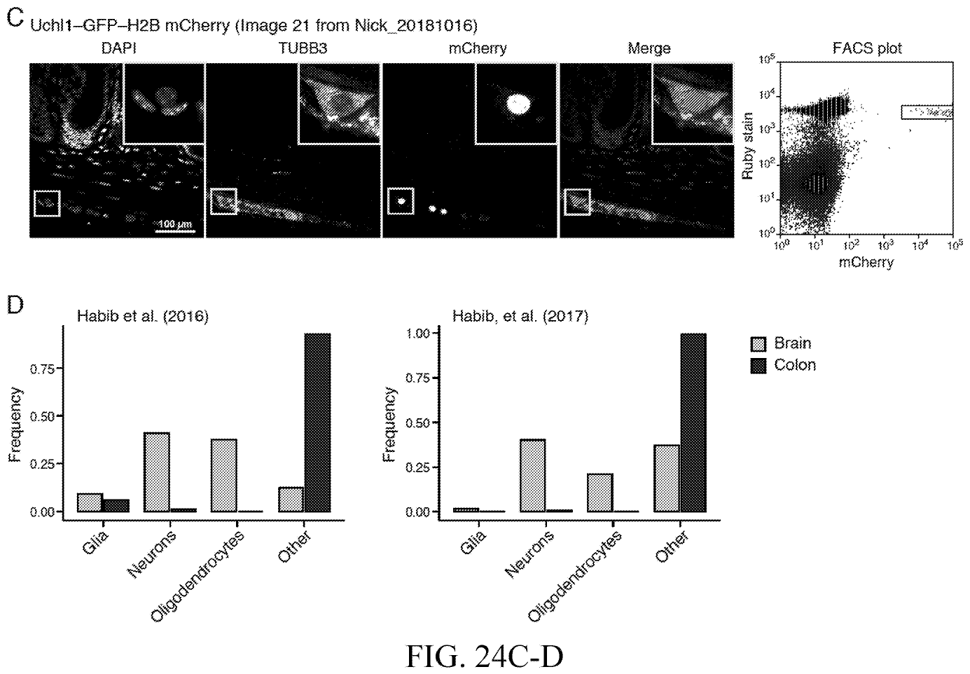

Because neurons comprise less than 1% of all colon cells, Applicants first devised a strategy to enrich for the mouse ENS. Applicants used three mouse models: (1) Wnt1-Cre2 (19) and (2) Sox10-Cre (20) transgenic mice, which are established Cre-drivers that efficiently label the neural crest (21, 22), and (3) Uchl1-Histone2BmCherry:GFP-gpi mice, which specifically labels neurons (23). For both Cre-driver mice, nuclei were tagged using the conditional INTACT (Isolation of Nuclei TAgged in specific Cell Types) allele (24). In all cases, Applicants extracted labeled nuclei, FACS-enriched them, and profiled them using SMART-Seq2 (17) (FIG. 20A,B, FIG. 24A-C).

Previously published snRNA-seq protocols (16, 17) did not perform well on ENS nuclei from the colon, in contrast to their excellent performance on labelled nuclei from the brain (FIG. 24D). In addition, the Wnt1-Cre2 driver mostly labeled non-ENS cells within the colon (FIG. 24B), and the Sox10-Cre driver labelled both neurons and oligodendrocytes in the brain (FIG. 24D), whereas Applicants anticipated recovering only brain oligodendrocytes (25). These limitations raised the need to develop new snRNA-seq approaches.

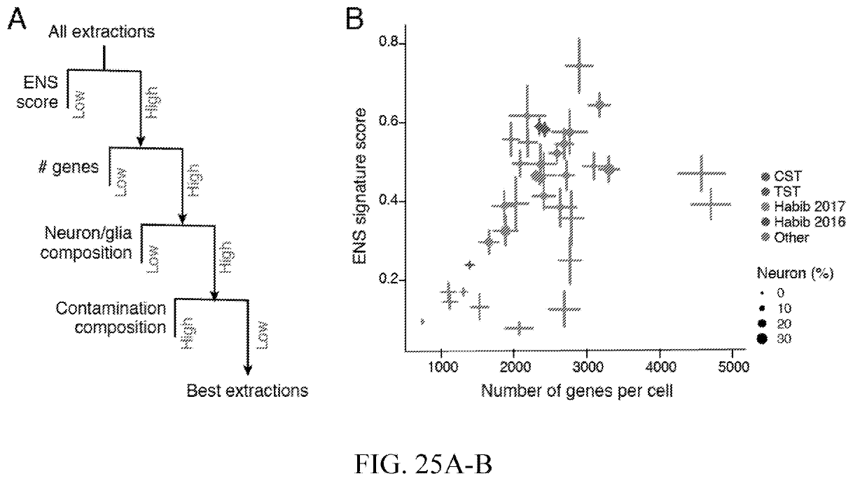

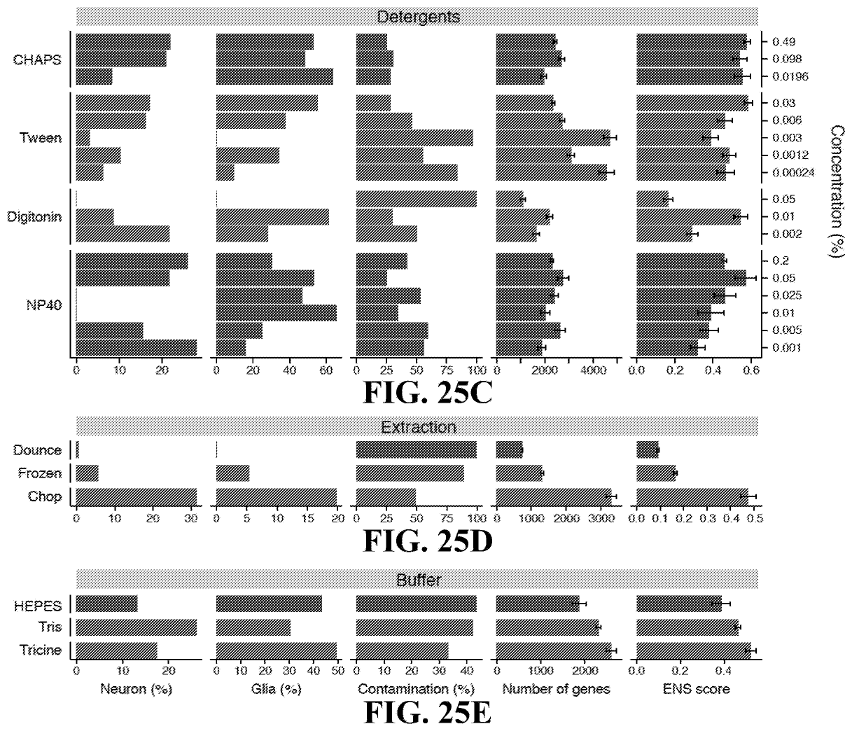

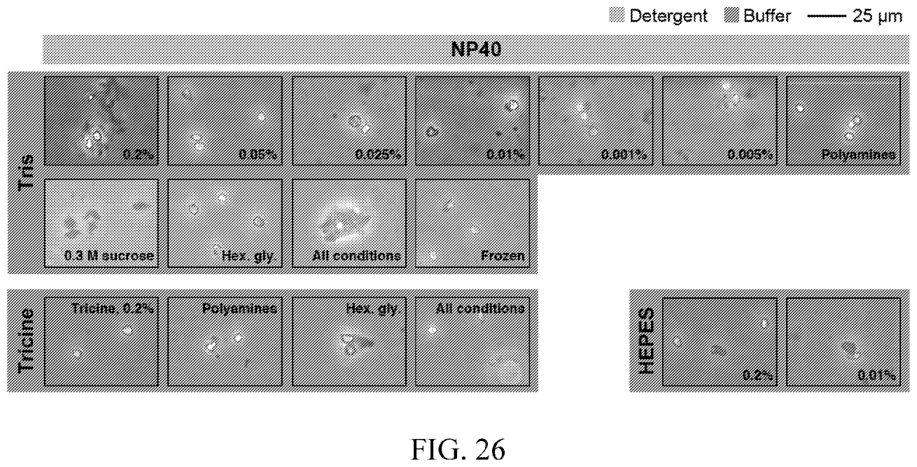

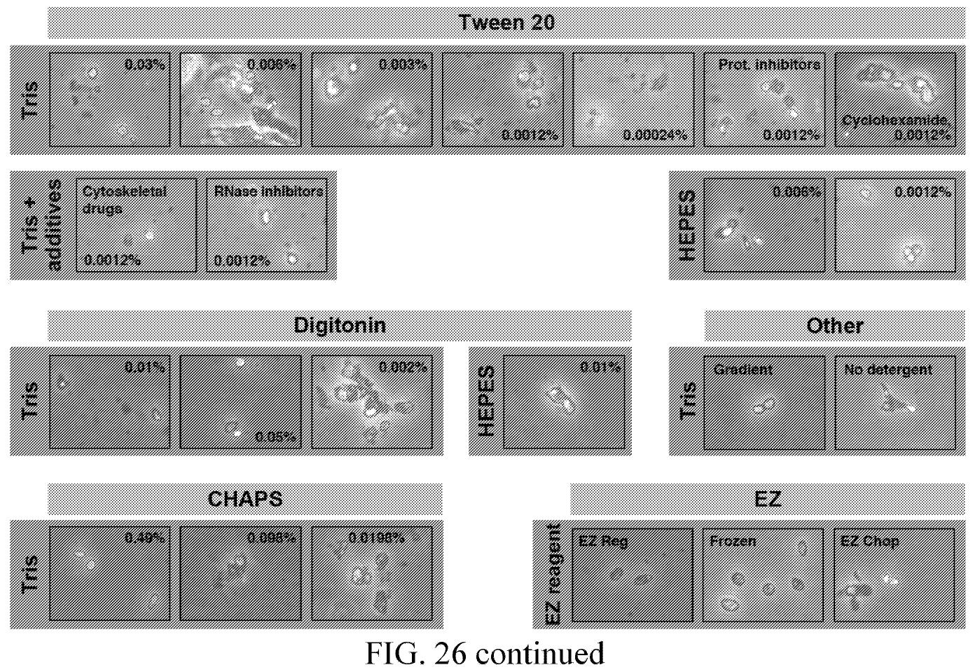

To develop snRNA-seq methods that are compatible with a broader range of tissues, including colon, Applicants performed an optimization with nuclei from adult Sox10-Cre; INTACT mice, systematically varying the detergent (NP40, CHAPS, Tween, or Digitonin), detergent concentrations, buffer (HEPES, Tris, Tricine), mechanical extraction conditions (dounced, chopped, or ground tissue), and added modifiers (e.g. salts, polyamines) used in nuclei isolation (SOM), and compared to published protocols (16, 17) (FIG. 25). Applicants profiled 5,236 nuclei isolated across 104 preparations spanning 36 extraction conditions (mean=145 nuclei per condition) using SMART-Seq2 (FIG. 20A; FIG. 25). Applicants scored conditions by (1) the recovery rate of neurons and glia relative to other cells (i.e. damaged or contaminating cells), (2) the number of genes detected per cell; and (3) an ENS signature score of known markers of enteric neurons and glia (FIG. 20C; FIGS. 25B-E and 26; SOM).

Detergent type, detergent concentration, buffer, and mechanical force each impacted quality metrics (FIGS. 25B-E and 26) and Applicants identified two conditions with high ENS recovery and low contamination rates (˜20% neurons, 55% glia, 25% contamination across both conditions, FIG. 20C), which also yielded high-quality profiles enriched in the ENS signature score (FIG. 25B-E). Applicants termed these preparations “CST” (0.49% CHAPS detergent, Salts, Tris buffer, and “chopped” tissue) and “TST” (0.03% Tween-20 detergent, Salts, Tris buffer, and “chopped” tissue). Both preparations yielded higher numbers of detected genes than published methods (mean=2,486 for CST and 2,542 for TST vs. 1,502 for published protocols on average across all nuclei; p<10-10 for both comparisons; Wilcoxon test).

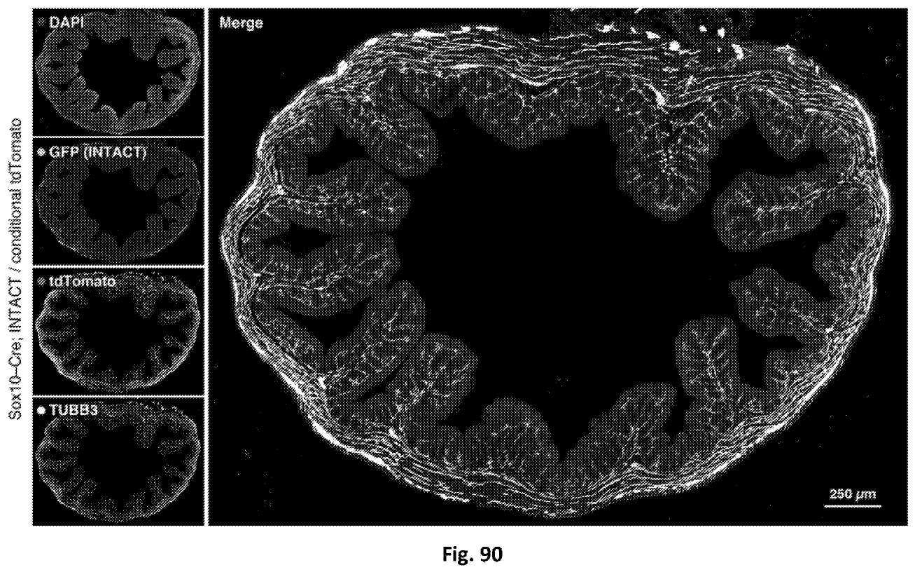

For all three transgenic lines, Applicants validated nuclei labeling within TUBB3+ neurons and confirmed their ability to enrich for extracted labeled nuclei using FACS (FIG. 24C). For the Sox10-Cre driver, Applicants confirmed extensive neuron labeling by generating a triple transgenic animal harboring Sox10-Cre, INTACT, and conditional tdTomato (Madisen et al., 2010) alleles, to label both the nuclei (i.e. INTACT) and cell bodies and their projections (i.e. tdTomato) of the ENS. There was excellent concordance between TUBB3 (neuron) immunostaining and reporter expression within the mouse colon (FIG. 90; tdTomato+/TUBB3− cells represent glia). For the Wnt1-Cre2 driver, Applicants observed labeled neuron nuclei, and also extensive signal in the colon mucosa (FIG. 24C); Applicants validated that the Wnt1-Cre2 driver also labeled colon epithelial cells by snRNA-seq. This off-target labeling may explain why a previous study using the Wnt1-Cre driver to target the ENS removed the mucosa when profiling enteric neurons of early post-natal mice with scRNA-seq (Zeisel et al., 2018). Lastly, for the Uchl1-H22B mCherry mice, Applicants observed labeling of enteric neurons but not of enterendocrine cells (the main neuroendocrine type in the intestine; Modlin et al., 2008), by histology (FIG. 24C) and snRNA-seq.

### Example 7

### Preservation of Ribosomes or Rough Endoplasmic Reticulum on the Nuclear Envelope Allows for Mature mRNA Capture

To understand the basis for these performance differences among nuclei preparations, Applicants compared nuclei structure between CST, TST, and published preparations for snRNA-seq (16, 17), using ultrathin-section transmission electron microscopy (TEM) (SOM, FIG. 20D). As expected, the two published methods yielded isolated intact nuclei (FIG. 20D). In contrast, CST preserved not only the nuclear envelope, but also the ribosomes (26) on the outer nuclear membrane (FIG. 20D); Applicants thus termed this method RAISIN (Ribosomes And Intact SIngle Nucleus) RNA-seq. TST maintained both the rough ER and its attached ribosomes (26) on the outer nuclear membrane (FIG. 20D); Applicants thus termed this method, INNER Cell (INtact Nucleus and Endoplasmic Reticulum from a single Cell) RNA-seq. Consistent with the TEM results, both RAISIN-RNA-seq and INNER-Cell RNA-seq yielded higher exon:intron ratios than the published methods (FIG. 20E; 41% and 64% increases, respectively), suggesting greater recovery of mRNA relative to pre-mRNA.

Of the two methods, Applicants opted to use RAISIN RNA-seq to profile the mouse and human ENS, because it captures more neurons and has fewer contaminants than INNER Cell RNA-seq (FIG. 20C; FIG. 25B-E). To test whether RAISIN RNA-seq is compatible with massively parallel droplet-based scRNA-seq, Applicants also sequenced 10,889 unsorted RAISINs from the mouse colon (SOM). Applicants recovered most major cell types in the colon, including epithelial cells, myocytes, fibroblasts, endothelial cells, immune cells, mesothelial cells, neurons, and glia (FIG. 20F), without any apparent “doublet” clusters, indicating that RAISINs correspond to single nuclei rather than to cellular aggregates. Therefore, even though RAISIN RNA-seq captures RNA both inside and outside the nuclear envelope, it is compatible with droplet-based scRNA-seq and yields little observed contamination.

### Example 8

### RAISIN RNA-seq Survey of the ENS from Adult Mice Identifies 24 Neuron and 3 Glia Subsets

Applicants used RAISIN RNA-seq with SMART-Seq2 to profile 5,181 high-quality transcriptomes from the ENS of 24 adult mice, spanning a range of ages (11-52 weeks), both males and females, and two phases of the circadian rhythm (morning or evening), and dividing each colon specimen into four equally sized segments along the proximal-distal axis to capture differences in anatomical location (FIG. 20A). Applicants initially used Wnt1-Cre2;INTACT and Sox10-Cre;INTACT mice to label both neurons and glia, and Uchl1-Histone2BmCherry:GFP-gpi mice to subsequently enrich for enteric neurons (FIG. 24A-C); however, because the Wnt1-Cre2 driver targeted mainly epithelial cells (FIG. 24B), Applicants focused on the other transgenic mouse models.

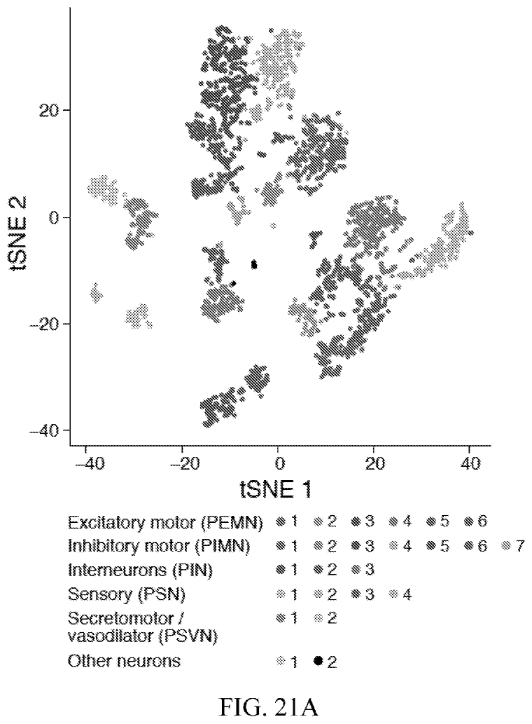

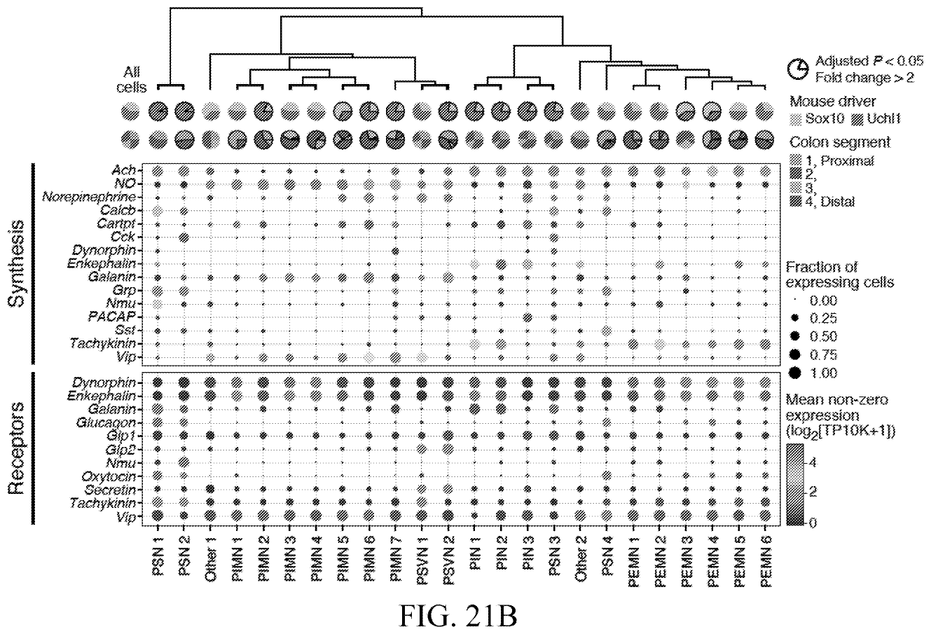

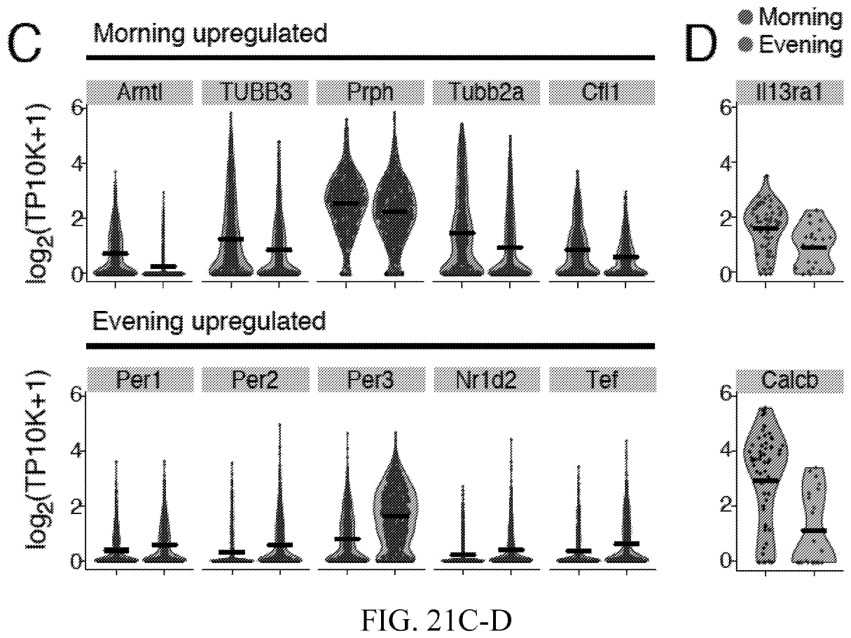

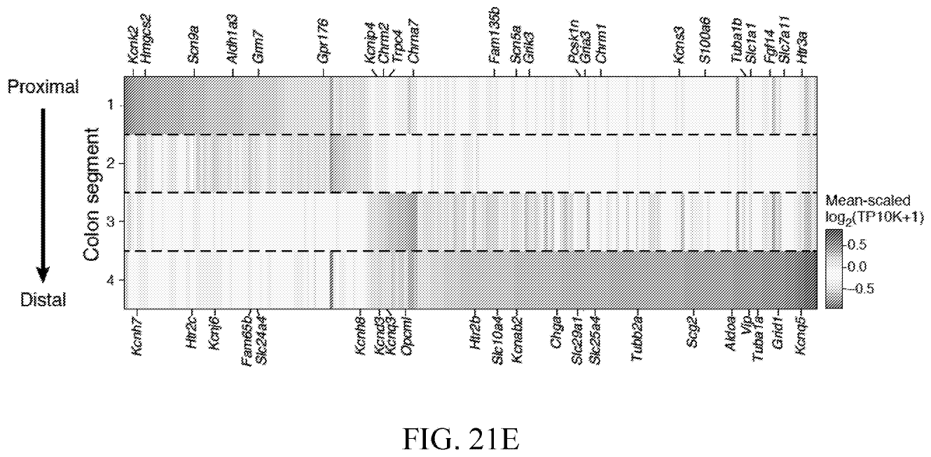

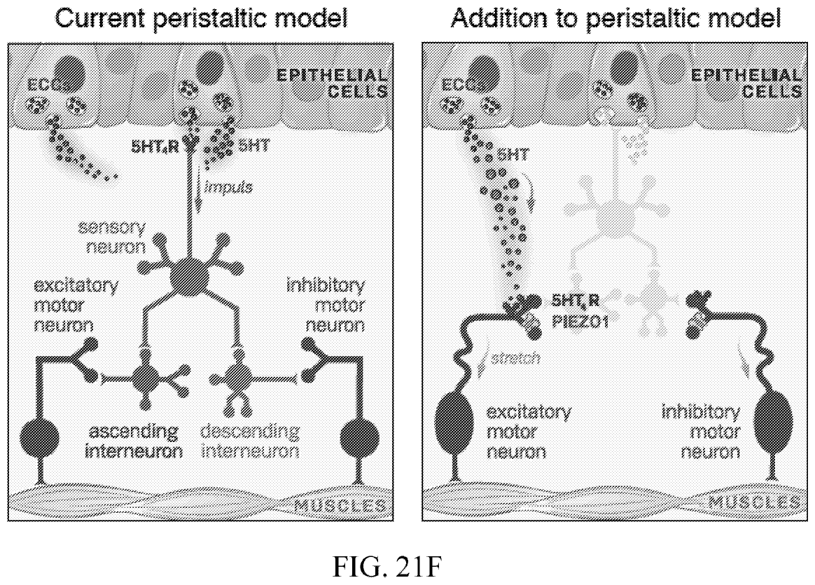

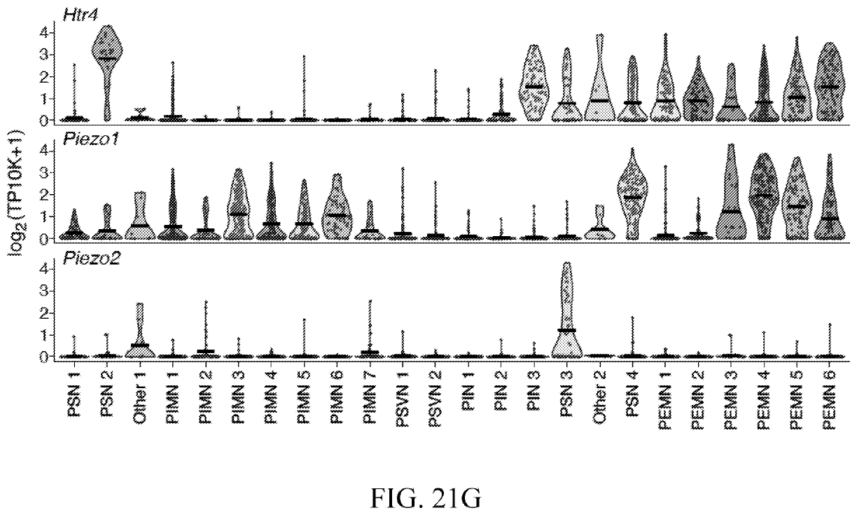

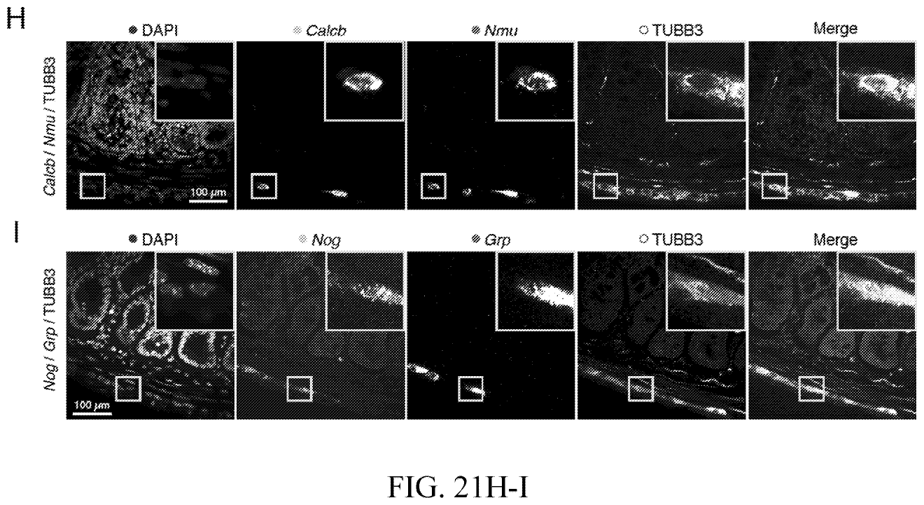

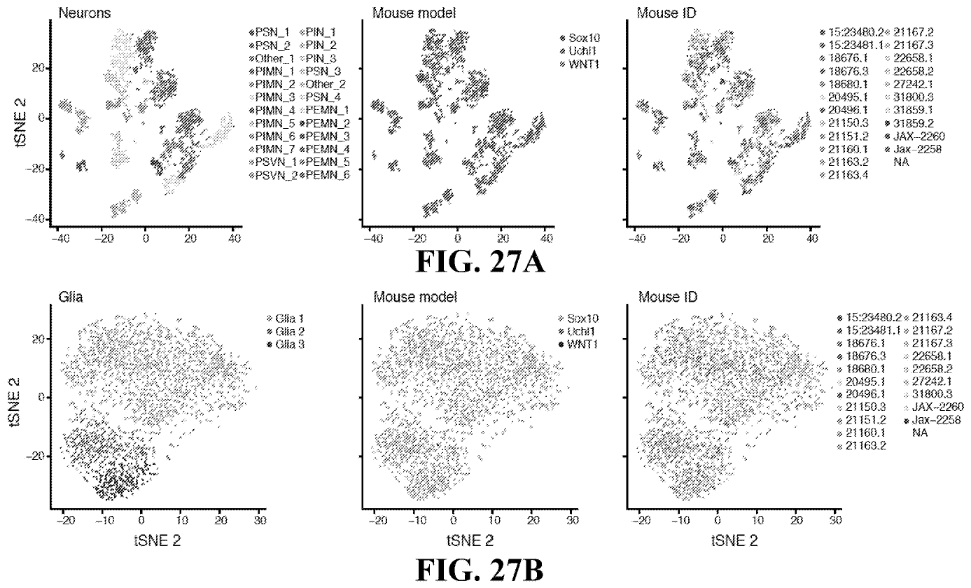

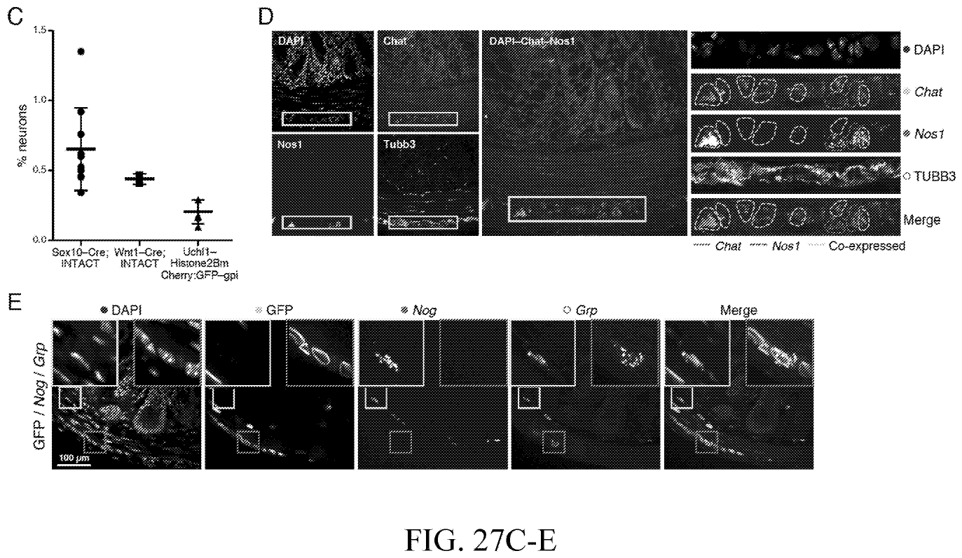

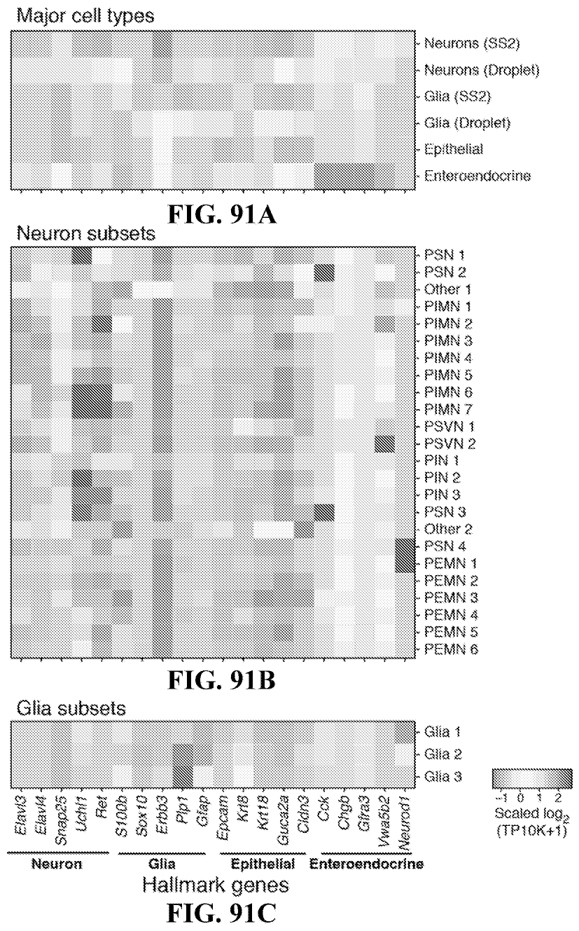

Among the 5,181 transcriptomes, Applicants identified 2,447 neurons and 2,710 glia (mean 7,491 and 4,732 genes per RAISIN, respectively), which Applicants clustered into 24 neuron and 3 glia subsets (FIG. 21A,B; FIG. 27A,B, table 18), arranged into a hierarchy (FIG. 21B), and annotated post-hoc by known marker genes (FIG. 21B; SOM), many of which Applicants validated in situ (FIGS. 21G,H, 27D,E 28). Of the 2,447 neurons and 2,710 glia identified, there was an average of 7,491 and 4,732 genes detected per RAISIN, respectively, which partitioned into 24 and 3 subsets, respectively (FIGS. 91A-91C; 21A, 27B). The clusters were enriched for markers of neuron and glia transcriptomes from scRNA-seq studies (FIGS. 91A, 91B) (Haber et al., 2017; Lasrado et al., 2017), with no detectable epithelial or enteroendocrine contamination, except for 8 contaminating cells in the “Other 2” cluster (FIG. 91A-91C). Neurons and glia clustered primarily by cell subsets, rather than by mouse, intestinal region, or other known technical covariates (FIG. 27A,B). Applicants estimate that enteric neurons comprise less than 1% of all nuclei in the murine colon after adjusting the numbers of FACS-sorted nuclei by the proportions of neurons identified in each mouse model (SOM) (FIG. 27C).

Broadly, neurons partitioned into either cholinergic (Chat+) or nitrergic (Nos1+) subsets (FIG. 21B, Ach and NO producing, respectively). As exceptions, four subsets expressed both Chat and Nos1 (defined as log 2(TP10K+1)>0.5), which Applicants validated in situ (FIG. 27D), and one subset expressed neither marker. Based on expression of known marker genes, Applicants defined putative neurons subsets (FIG. 21A,B), including: (1) Chat+Tac1+ excitatory motor neurons (PEMNs; 6 subsets), and (2) Nos1+Vip+ inhibitory motor neurons (PIMNs; 7 subsets), which together coordinate muscle contraction and relaxation; (3) CGRP+ sensory neurons (PSNs; 4 subsets), which sense and respond to chemical and mechanical signals in the intestine; (4) interneurons (PINs; 3 subsets), which relay signals between neurons; and (5) Glp2r+ secretomotor and vasodilator neurons (PSVNs; 2 subsets), which trigger secretions and fluid movement in other cell types.

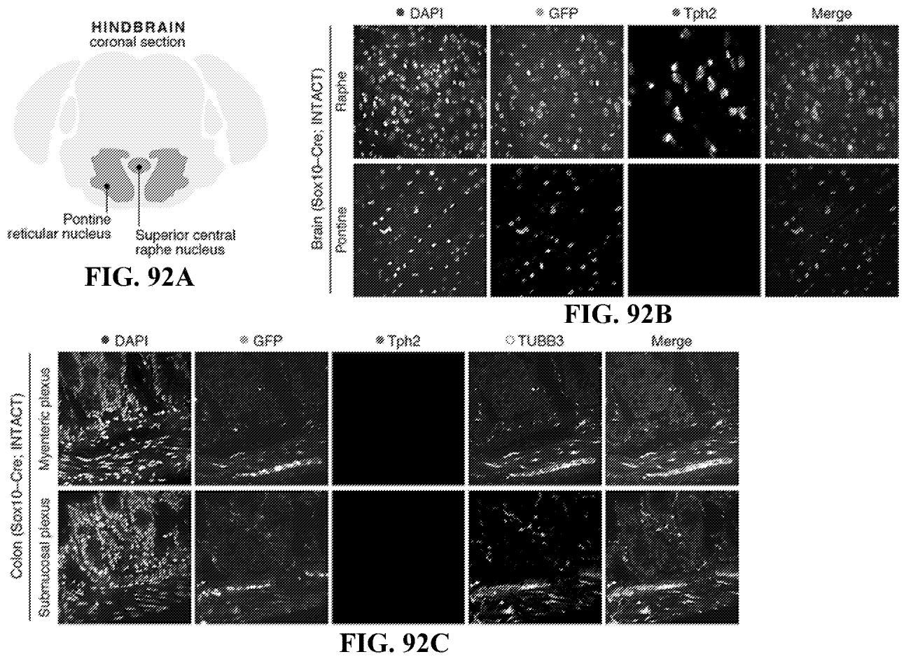

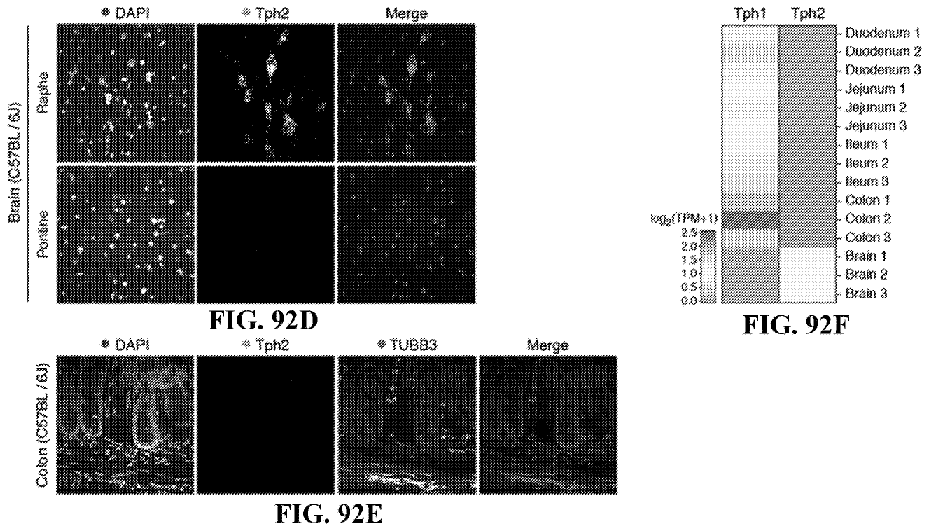

The only major marker that Applicants could not detect was the neuronal enzyme for serotonin synthesis, Tph2 (Gershon, 2009; Mawe and Hoffman, 2013). Applicants probed for Tph2 in situ in the colon as well as targeted brain regions, which served as positive (raphe nuclei) and negative (pontine reticular nucleus) controls (FIG. 92A-92E), but only observed Tph2 signal in the brain. Applicants considered the possibility that Tph2-expressing enteric neurons are rare (Costa et al., 1982, 1996), and examined published bulk RNA-seq data (Soliner et al., 2017), finding Tph2 expression in the brain, but not the colon (FIG. 92F). Lastly, an independent scRNA-seq study of the small intestine myenteric plexus did not yield serotonergic neurons (Zeisel et al., 2018). However, Applicants cannot exclude the possibility that Tph2 is expressed only under different physiological conditions, in other locations, or cannot be captured using current genomic and RNA-FISH tools. One possibility is that serotonergic neurons only populate the small intestines, as conditional Tph1 knock-out mice crossed with a Villin-Cre driver, which lack serotonin production by the mucosa, have detectable serotonin in the duodenum and jejunum; although these regions still had detectable Tph1 mRNA in the conditional knock-out (Kim et al., 2018).

### Example 9

### ENS Composition and Expression Programs Vary by Region and with Circadian Oscillations

To systematically assess sources of variation in the ENS, Applicants leveraged the fact that the atlas comprises samples that vary by genetic background, age, sex, circadian time point, and anatomical location, to test how each factor impacts ENS composition (i.e. the relative proportions of neuron subsets) or gene expression within each neuron subset.

The transgenic background had profound effects on neuron composition (FIG. 21B; FIG. 27A), suggesting distinct developmental origins for some neuron subsets. In particular, two subsets of putative sensory neurons (PSN1 and PSN2) were nearly absent from Sox10-Cre mice (FIG. 21B), suggesting they may arise from distinct lineages (20, 27). ENS composition also varied significantly along the length of the colon within each of the Sox10 and Uchl1 lines, with distinct neuron subsets enriched in different regions (FIG. 21B). For example, PSN1 and PSN2 were enriched in the proximal colon (P<10-22 and 10-6, respectively; Fisher's exact test), whereas distinct subsets of putative motor neurons (PMNs) were enriched in either the proximal or distal colon (FIG. 21B).

Applicants next used a regression framework to identify genes that were differentially expressed (DE) with respect to age, sex, circadian phase, and colon location, in a manner shared across neuron subsets (SOM). Overall, few DE genes were associated with age or sex (with the exception of genes on the X and Y chromosomes) (Table 18); however, the circadian clock and colon location had substantial impacts on gene expression of many neuron genes (table 18). For example, core clock regulators were among the most DE genes during morning (Arnt1) and evening (Per1, Per2, Per3) (FIG. 21C). In the morning, there was also increased expression of cytoskeleton-associated genes (e.g., Tubb3, Prph, Tubb2a, Cfl1), suggesting circadian regulation of structural remodeling (28), and genes involved in neuronal signaling (e.g., Scg2, Pcsk1n, and Slc7a11). In PSN1 and PSN2, Applicants also observed morning upregulation of genes involved in neuro-immune signaling (e.g., Calcb, Il13ra1) (FIG. 21C) (29,30). In the evening, several TFs were upregulated relative to morning, including Nr1d2, Tef, Rfx2, and Dbp (FIG. 21C), many of which are known circadian regulators (31).

In addition, there were significant changes in gene expression across colon regions, after controlling for differences in ENS composition (which itself varies by location) (FIG. 21D). Most notably, neurons in the distal mouse colon had higher expression of several neurotransmitter receptors, including serotonin receptors (Htr3a, Htr3b), glutamate receptors (Gria3, Grid1), acetylcholine receptors (Chrna7, Chrm1), and potassium and sodium channels (Kcnq5, Scn5a), suggesting electrophysiological differences along the ENS.

### Example 10

### Motor Neuron Expression Profiles Suggest that Mechanosensation Drives the Peristaltic Reflex

The myenteric plexus is a major functional unit of the ENS, moving luminal contents along the intestine through coordinated muscle contraction and relaxation (13). The canonical model of the peristaltic reflex (FIG. 21E, left) (13) begins with the release of serotonin (5HT) by enterochromaffin cells, which acts on sensory neurons via the 5HT receptor 4 (HTR4). Interneurons then relay this signal to ascending and descending motor neurons, which elicit muscle contraction and relaxation, respectively (13). This model is based on associations between muscle contraction and serotonin release, but was recently challenged, because neither ablation of serotonin synthesis in enterochromaffin cells nor mucosa removal abrogate muscle contraction (32). Applicants therefore hypothesized that the molecular signatures of neuron subsets could help build and test models of peristalsis.

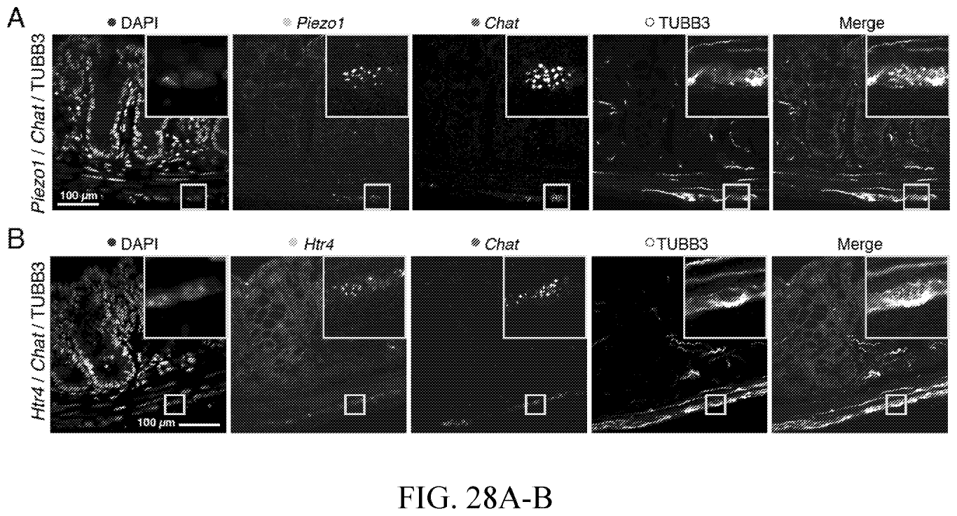

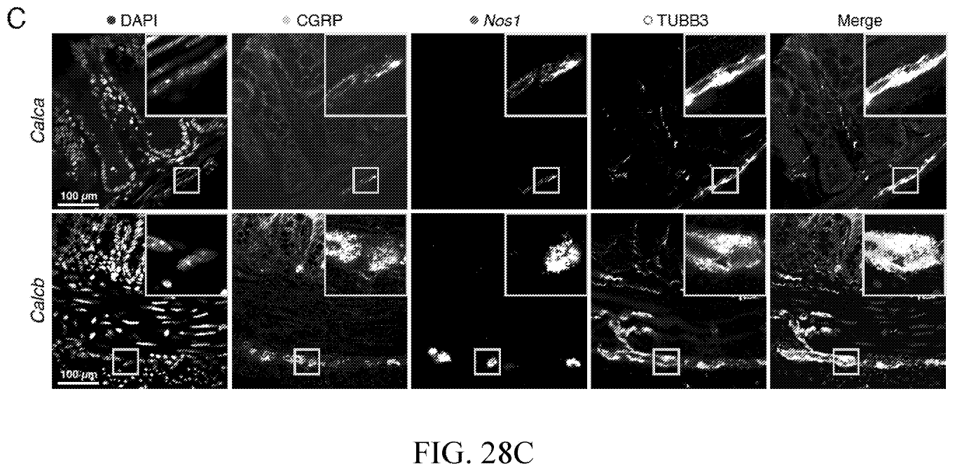

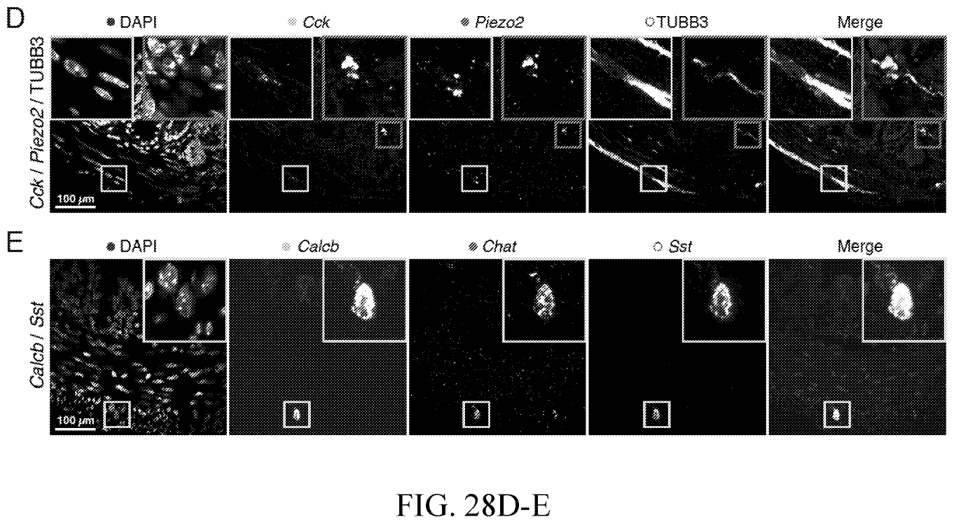

The transcriptional profiles of putative motor neurons suggest revisions to the peristaltic model, with a possible role for the mechanosensation of gut distention in driving peristaltic reflexes (FIG. 21F, right). First, nearly all putative motor neurons express the mechanosensitive ion channel, Piezo1 (FIG. 21G, PEMNs and PIMNs; confirmed in situ, FIG. 28A), suggesting they have the capacity to directly sense distention. Mechanosensation in the GI tract is currently attributed to enterochromaffin cells, with speculation that some interneurons and intestinofugal neurons are also mechanosensitive (33). However, expression of Piezo1 in putative motor neurons, and the dispensability of mucosal serotonin for smooth muscle activity, raises the hypothesis that peristalsis is at least partially driven by distention, specifically via motor neuron depolarization through Piezo1.

Moreover, although the peristaltic model posits that enterochromaffin cells act on sensory neurons via serotonin receptor 4 (Htr4) (FIG. 21F, left) (13), Htr4 is expressed by putative excitatory motor neurons (PEMNs), and Applicants confirmed this in situ in Chat+ neurons of the myenteric plexus (FIG. 28B). This suggests that serotonin may be able to act directly on motor neurons rather than only via sensory and interneuron intermediates.

### Example 11

### Sensory Neurons Express Key Regulators of ILC Responses and Tissue Homeostasis

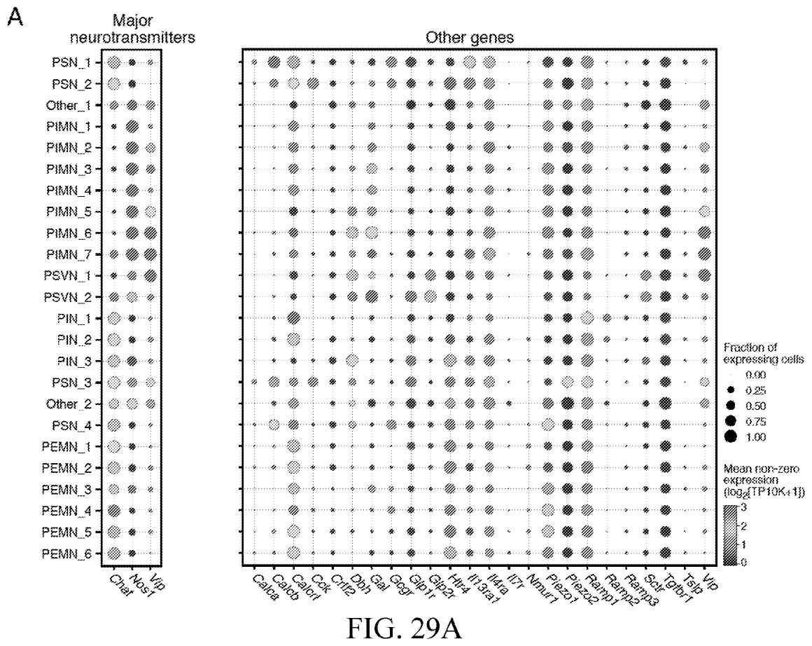

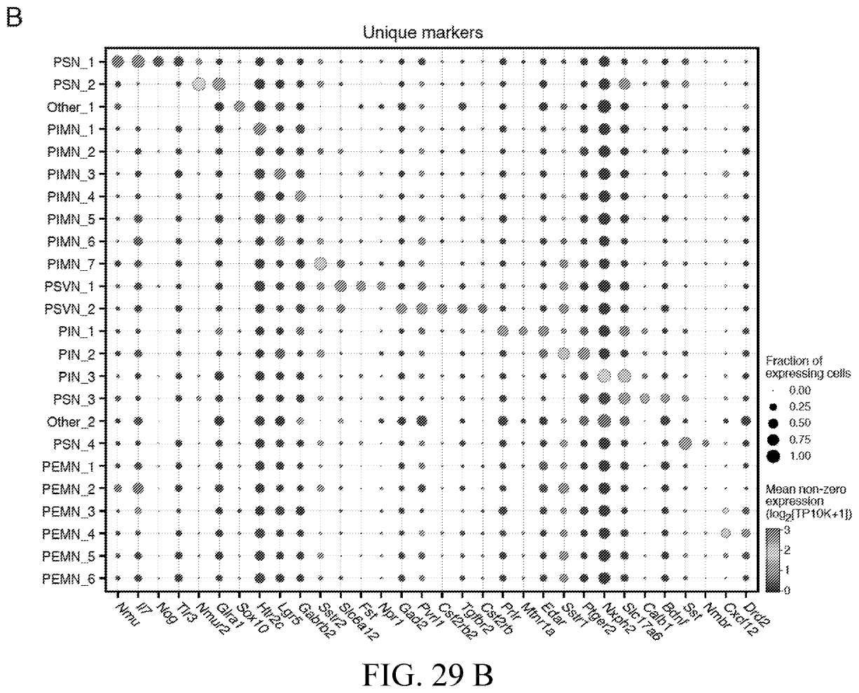

Applicants identified four subsets of putative sensory neurons (PSNs) by expression of calcitonin gene-related peptide (CGRP), a marker of sensory neurons expressed in two forms (Calca, Calcb), which is involved in feeding, pain sensation, hormone secretion, and inflammation (34). While all four subsets express Calcb, only PSN3 expresses Calca at significant (but low) levels (FIG. 29A), which Applicants confirmed in situ (FIG. 28C). The CGRP receptor (Calcr1) and one of its three co-receptors (Ramp1) are expressed in all neurons, except putative secretomotor neurons (FIG. 29A).

Applicants inferred the likely target cells for each PSN subset based on the signaling molecules and receptors that they express (FIG. 21B, table 18, FIG. 29A,B). For example, most sensory neuron subsets express receptors for glucagon (Gcgr), glucagon-like peptide 1 (Glp1r), and galanin (Galr) (FIG. 21B; FIG. 29A), peptides that are produced by enteroendocrine cells with roles in hunger and satiety (35). One subset, PSN3, co-expresses Cck and Vip (FIG. 21B), markers of intestinofugal neurons that innervate the prevertebral ganglia (36), thus supporting connections to the sympathetic nervous system. This subset also uniquely expresses brain-derived neurotrophic factor (Bdnf, FIG. 29B), which is elevated in patients with irritable bowel syndrome (IBS), where it is correlated to abdominal pain (37), and Piezo2 (FIG. 21G), a mechanosensitive ion channel, which may help detect and regulate smooth muscle tone (38) (confirmed in situ; FIG. 28D). Another Calcb+ subset, PSN4, uniquely expresses somatostatin (Sst, FIG. 21B, FIG. 29B) (validated in situ; FIG. 28E), previously attributed to interneurons (13); the role of SST in the GI tract is poorly understood, but has been broadly linked to regulating most GI functions, including motility, secretion, absorption and the sensation of visceral pain (39). Localization of Sst expression to a single neuron subset now empowers dissection of its function in the ENS.

One sensory neuron subset, PSN1, uniquely expresses Noggin (Nog) and Neuromedin U (Nmu) (FIG. 21B), validated in situ (FIG. 21H,I): both genes are known key regulators of epithelial stem cells (40) and immune cells (9), respectively. In particular, Noggin is a BMP antagonist that is necessary for maintaining the intestinal stem cell niche, but whose cellular source is unknown. Noggin expression by sensory neurons raises the hypothesis that these neurons could help regulate the positioning or differentiation of intestinal stem cells. Furthermore, the neuropeptide NMU regulates type 2 cytokine responses via activation of innate lymphoid cells (ILCs) (9). Expression of its receptors, Nmur1 and Nmur2, on excitatory motor (PEMN1, PEMN2; FIG. 29A) and sensory (PSN1, PSN2, PSN3; FIG. 29B) neurons, respectively, suggests diverse neuronal targets of NMU, that may help orchestrate inflammation. PSN1 cells also express additional genes that may interact with ILCs, including Calcb, both subunits of the Il-13 receptor (Il4ra and Il13ra1, FIG. 29A), and Il-7 (FIG. 29B), a major regulator of ILC differentiation and survival (41). Lastly, both PSN1 and PSN2 cells express gastrin-releasing peptide (Grp, FIG. 21B), which in the lung is produced by neuroendocrine cells and contributes to the response to tissue injury (42).

### Example 12

### Secretomotor Neurons may Integrate Epithelial and Immune Signals

Secretomotor/vasodilator neurons (SVNs) integrate signals from the mucosa and sympathetic ganglia to regulate fluid movement between the body and the lumen. Applicants identified two subsets of putative secretomotor/vasodilator neurons (PSVNs) corresponding to non-cholinergic (PSVN1) and cholinergic (PSVN2) subtypes (43) (FIG. 21A,B). Both subsets uniquely express receptors for GLP-2 (Glp2r) and secretin (Sctr), hormones released by enteroendocrine cells that stimulate blood flow (44) and epithelial secretions (45), respectively (FIG. 21B; FIG. 29A). Most local reflexes regulating water and electrolyte balance likely act through non-cholinergic SVNs (43), and the data suggest that cholinergic SVNs may support tissue homeostasis. Specifically, the GM-CSF receptor (Csf2rb, Csf2rb2, FIG. 29B) and Thymic Stromal Lymphopoietin (Tslp, FIG. 29A) are expressed by PSVN2s, suggesting these neurons participate in GI immune responses (46, 47).

### Example 13

### Profiling the Human Muscularis Propria Using RAISIN RNA-seq

Next, Applicants profiled human colon enteric neurons. Unlike genetic mouse models, Applicants could not enrich for nuclei from human enteric neurons, and thus opted to profile the muscularis propria (MP), which has a higher proportion of neurons than the submucosa or mucosa. Applicants isolated and profiled nuclei from cancer-adjacent normal colon segments from colorectal cancer resections from both genders (5 male, 5 female) and a range of ages (35-90) (Tables 19-22). Based on the mouse data (FIG. 27C), Applicants conservatively estimated a 0.5% capture rate for neuron nuclei, such that in order to capture 500 human neurons, Applicants would need to profile at least 100,000 unsorted nuclei.

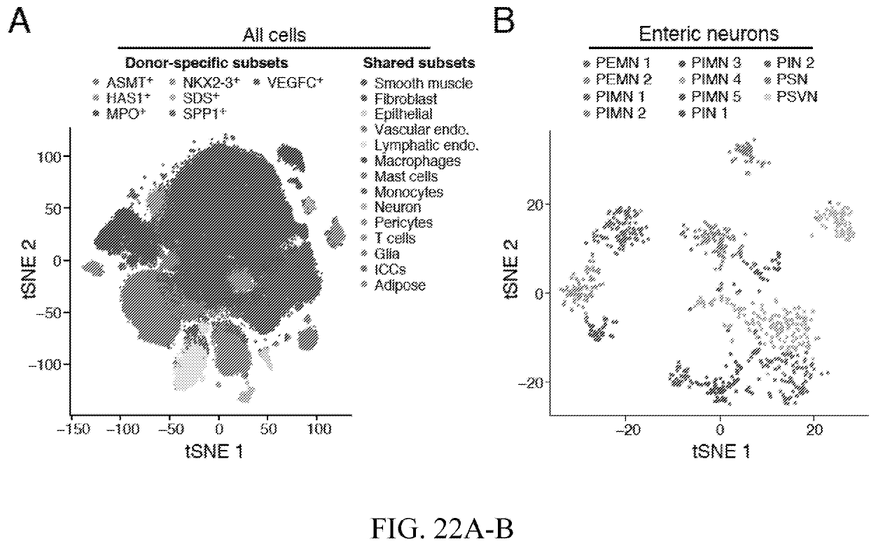

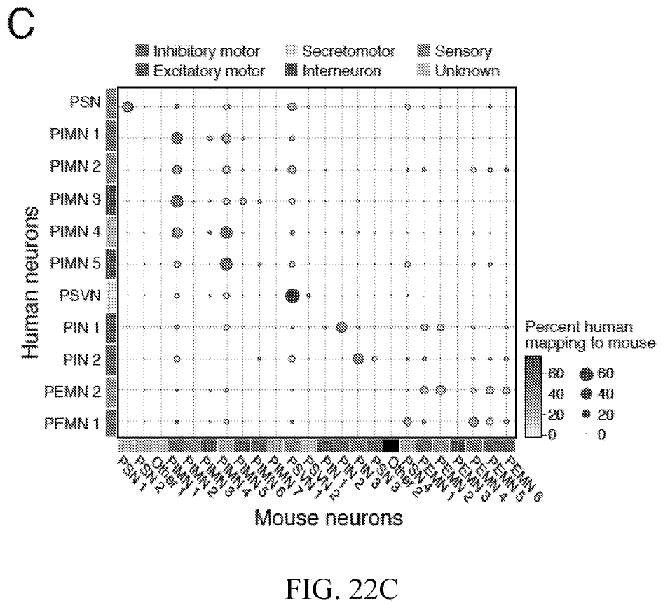

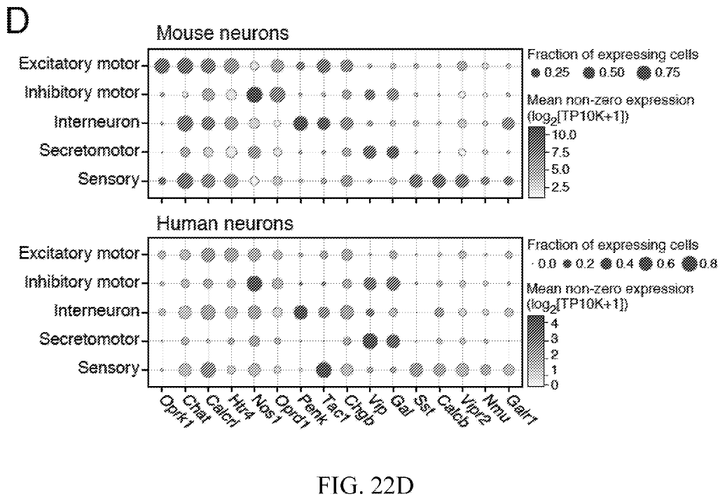

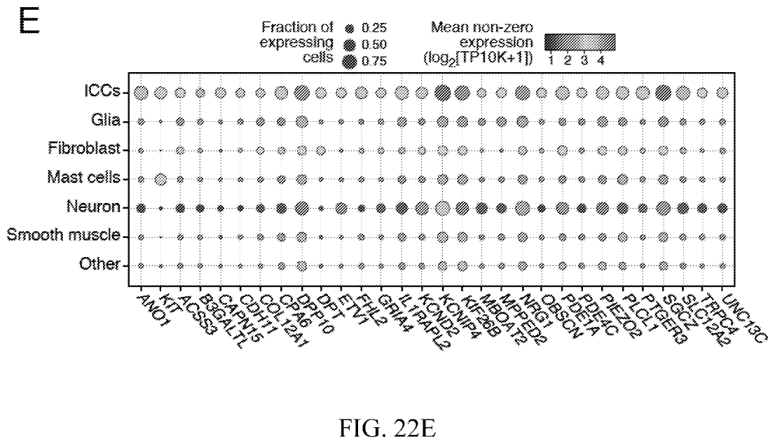

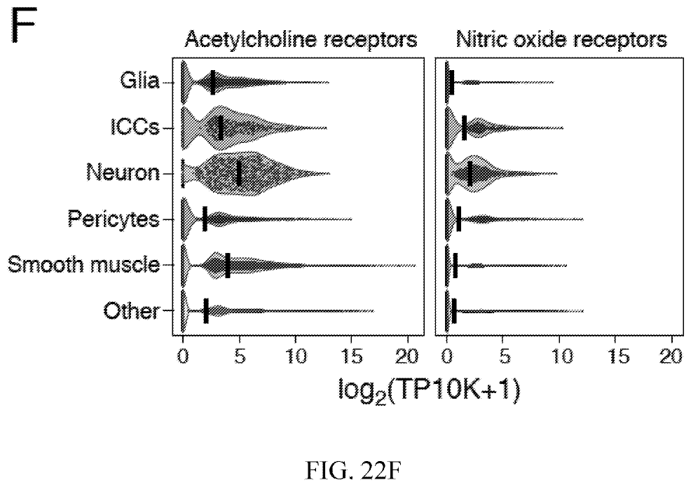

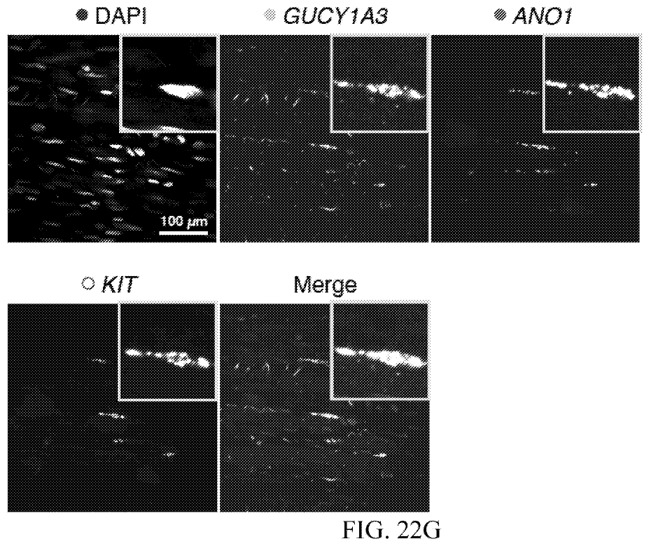

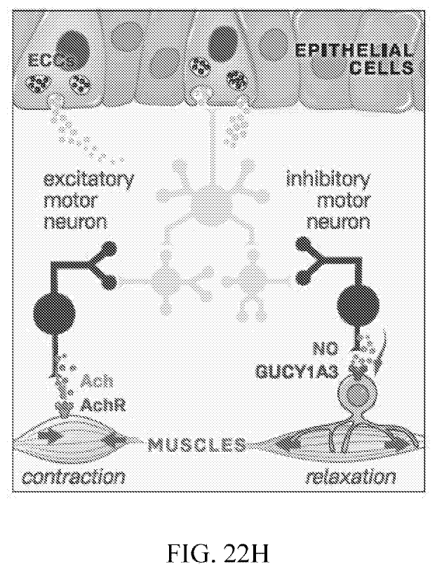

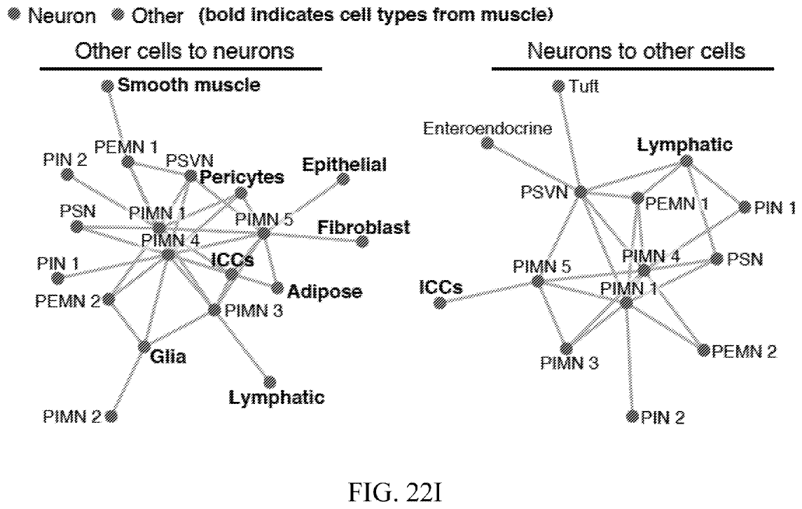

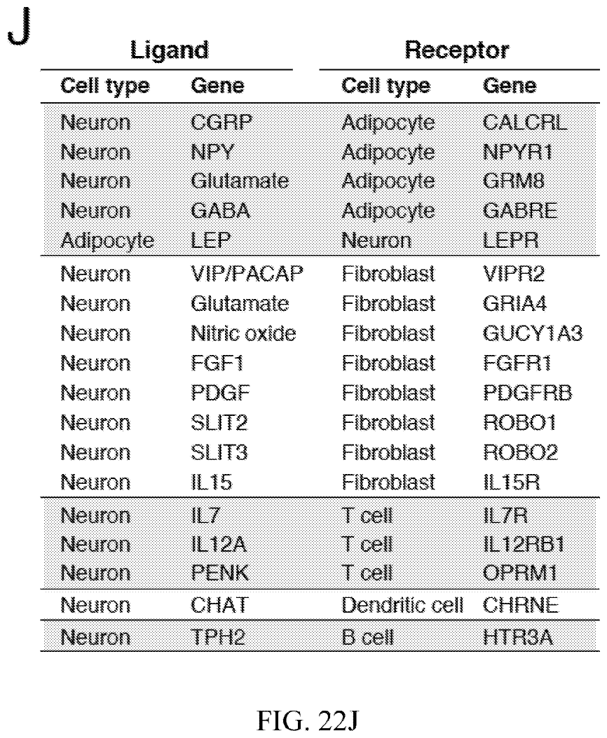

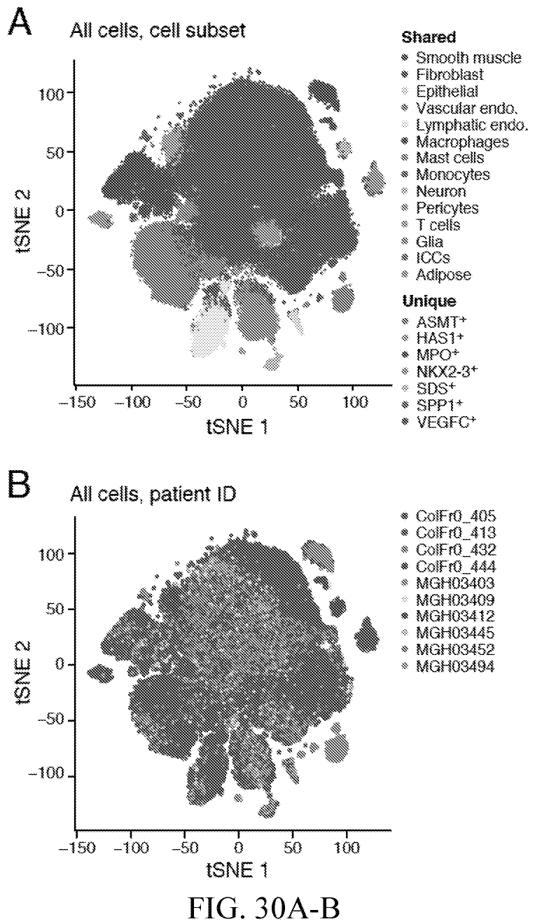

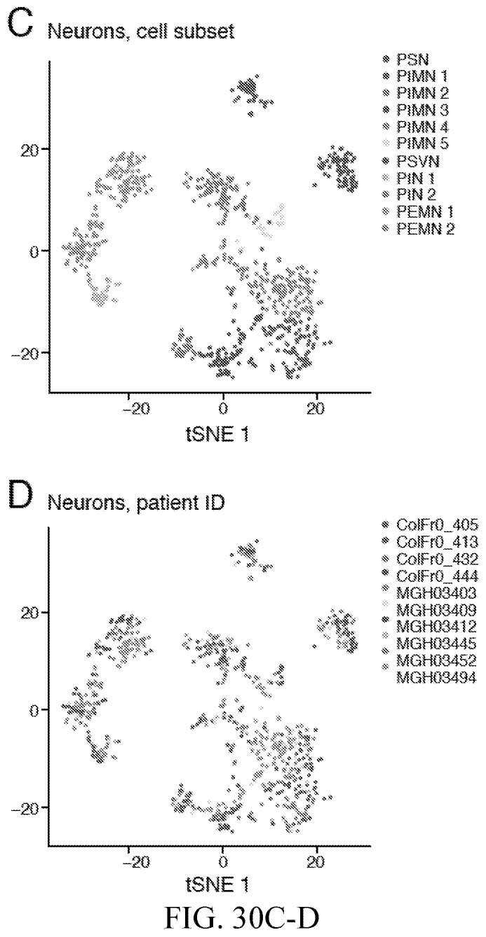

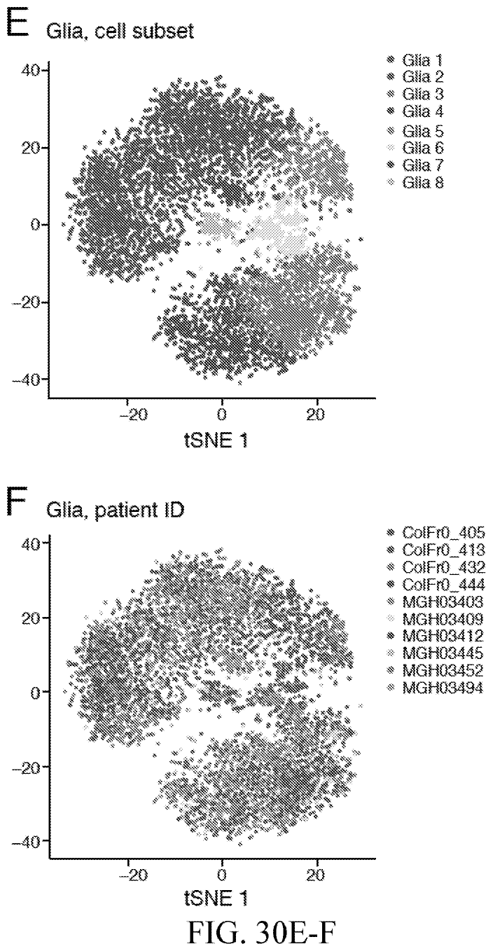

Profiling 134,835 human RAISINs from the muscularis propria recovered transcriptomes from neurons, adipocytes, endothelial cells (lymphatic, vascular), fibroblasts, glia, immune cells (macrophages, mast cells, lymphoid cells), interstitial cells of Cajal (ICCs), myocytes, and pericytes (FIG. 22A), each annotated by expression of known marker genes (FIG. 30A; Tables 19-22). Some subsets were enriched in specific patients (FIG. 30A-F), which may be due to differences in sampled locations, variable cellular states or variation in the sampling of rare cells. Additionally, human RAISIN RNA-seq data contained more background contamination than either mouse RAISIN SMART-Seq2 or droplet data (data not shown), possibly due to delayed tissue freezing time following resection.

### Example 14

### Human Enteric Neurons Cluster into 11 Subsets with Distinct Transcriptional Programs

The 134,835 RAISINs include 831 human enteric neurons (0.6%), which clustered into 11 subsets (FIG. 22B) after correcting for putative differences in cell quality (FIG. 30G-J; SOM). Notably, the neuron recovery rate in humans slightly exceeded Applicants original estimate, likely because the muscularis propria is enriched for neurons relative to the rest of the colon.

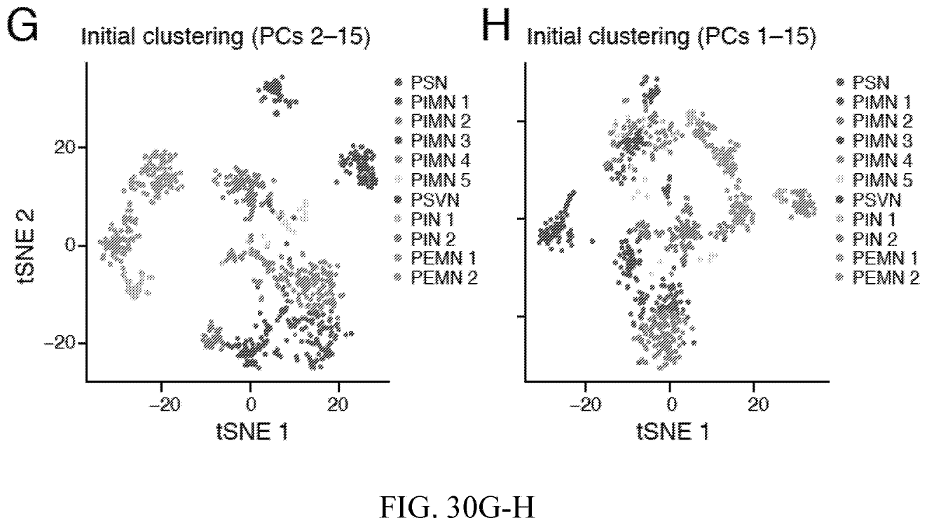

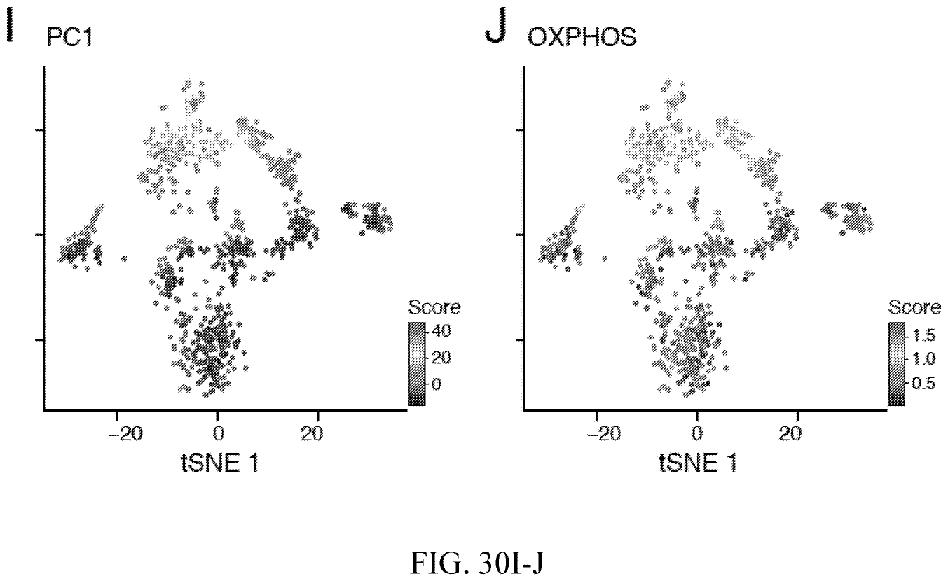

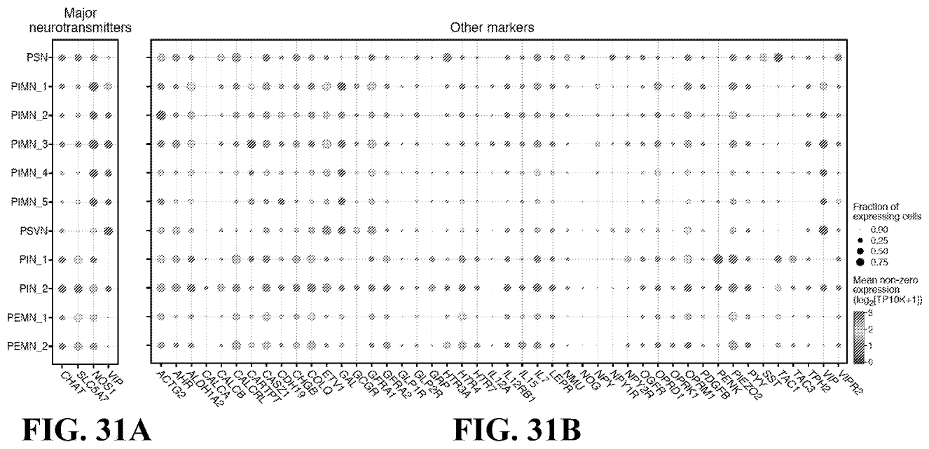

Although Applicants detect many hallmark neurotransmitters, CHAT was lowly expressed (FIG. 31A), either due to actual low expression in human cells, reduced levels in the nucleus, or cancer-adjacent effects. Applicants do detect the SLC5A7 (FIG. 31A), a transporter that mediates choline uptake into cholinergic neurons (48), which is co-expressed with Chat in mouse neurons. Applicants therefore used SLC5A7 as a surrogate marker for CHAT in human neurons. Interestingly, Applicants observed broad, albeit low, levels of expression of tryptophan hydroxylase 2 (TPH2; required for serotonin biosynthesis) across almost all human neuron subsets (FIG. 31B), but not in mouse neurons (data not shown), suggesting differences in serotonergic signaling between the two species.

### Example 15

### Human ENS Contains Sensory, Motor, Interneuron, and Secretomotor/Vasodilator Subsets that Share Core Transcriptional Programs with Mouse

Applicants used a classification-based approach (SOM) to map the 11 subsets of human neurons onto the 24 mouse subsets (FIG. 22C), leveraging the larger number of cells and deeper sequencing data in mouse to annotate the human cells. Applicants identified 2 PEMN subsets, 5 PIMN subsets, 1 PSN subset, 2 PIN subsets, and 2 PSVN subsets (FIG. 22B) and confirmed these annotations with known markers (FIG. 31A,B). Despite representing distinct regions of the colon (i.e. full colon vs. muscularis propria), both species contained similar neuron compositions, with excitatory and inhibitory motor neurons being the most abundant classes (FIG. 22C). However, sensory neurons were more abundant and more diverse in mouse. This may be due to removal of the human submucosa: humans contained only one sensory subset, whereas mice contained four (although Applicants cannot entirely rule out the possibility that the different number of profiled neurons may contribute to this difference as well). Furthermore, while the fraction of secretomotor/vasodilator neurons was similar across both species, the human muscularis propria lacked the cholinergic subtype, whereas mice contained both cholinergic and non-cholinergic subsets.

Applicants leveraged the human-mouse mapping to identify conserved (core) programs (FIG. 22D; Table 23; SOM) for each of five major neuron types. For example, the core transcriptional program for excitatory motor neurons (n=75 genes) includes acetylcholine, various receptors (e.g., GFRA2, OPRK1, HTR4), solute transporters (e.g., SLC5A7), transcription factors (e.g., CASZ1), and COLQ, which tethers acetylcholinesterase within the neuromuscular junction (49) (FIG. 22D, FIG. 31B, Table 23). In addition, human PEMNs uniquely express the mechanosensitive ion channel, PIEZO2 (FIG. 31B), whereas mice express Piezo1 (FIG. 21G). Similarly, Applicants defined core transcriptional programs for inhibitory motor neurons (n=89 genes; e.g., VIP, NOS1, CARTPT, GFRA1, OPRD1, ETV1), sensory neurons (n=76 genes; e.g., CALCB, NMU, NOG, SST, VIPR2), interneurons (n=57 genes; e.g., PENK, TAC1, ADRA2A), and secretomotor/vasodilator neurons (n=46 genes; e.g., VIP, GAL, SCGN, CALB2) (FIG. 22D; Table 23).

### Example 16

### Human Interstitial Cells of Cajal (ICCs) may Underlie Smooth Muscle Relaxation

Applicants' reference map of the human muscularis propria includes 431 KIT⁺ANO1⁺ ICCs (FIG. 22A; FIG. 30A), which are regarded as pacemaker cells that rhythmically alter the excitability of smooth muscle tissue (50, 51). Two major models exist for ICC function (50): either (1) neurons signal directly to smooth muscle, with an indirect role for ICCs (e.g., to generate motor patterns), or (2) neurons signal to ICCs, which then relay signals to smooth muscle to coordinate peristalsis.

To distinguish between these possibilities, Applicants defined a gene signature for ICCs (FIG. 22E) and mapped known ligand-receptor pairs onto neurons, ICCs, and smooth muscle cells (SOM). Although motor activity requires both excitatory (i.e. cholinergic) and inhibitory (i.e. nitrergic) signals to elicit contraction and relaxation, respectively, smooth muscle cells only expressed the receptors for acetylcholine (FIG. 22F). In contrast, the receptor for nitric oxide were expressed by ICCs (FIG. 22F), which Applicants validated in situ (FIG. 22G). As a positive control, Applicants note that nitric oxide receptors are detected in pericytes (FIG. 22F) (52). These results suggest a revised model of smooth muscle function, where enteric neurons directly activate smooth muscle contraction, but elicit smooth muscle relaxation indirectly via ICCs (FIG. 22H). Consistent with this hypothesis, smooth muscle-specific knockout of the B1 subunit of the nitric oxide receptor only partially reduces relaxation, whereas its global knockout nearly abolishes relaxation (53).

### Example 17

### Enteric Neurons Interact with Diverse Stromal and Immune Cells in the Colon

To systematically examine interactions between the enteric nervous system and other cell types in the human colon, Applicants analyzed profiles from the 134,835 RAISINs from the muscularis propria (above) together with 115,517 single cells from the colon mucosa (i.e. epithelium and lamina propria) (54). In total, these data include a wide range of cell types in the human colon, including 16 epithelial subsets, 26 immune subsets (myeloid and lymphoid), 7 endothelial subsets, 9 fibroblast subsets, myocytes, ICCs, adipocytes, 2 glia subsets (muscularis propria and lamina propria), and 11 neuron subsets. Applicants mapped thousands of receptor-ligand pairs onto this dataset and identified pairs of cell subsets expressing a significantly greater number of cognate receptor-ligand pairs than is expected under a null model (FIG. 22I; SOM).

Broadly, neurons were enriched for interactions with other cells from the muscularis propria rather than from the mucosa, suggesting the recovery of local interactions. This approach highlighted known interactions between excitatory motor neurons and smooth muscle (13), secretomotor/vasodilator neurons and both epithelial cells (i.e. tuft and enteroendocrine) and lymphatics (2), and glia and multiple subsets of neurons (FIG. 22I).

More unexpectedly, Applicants found statistically enriched interactions between neurons and diverse stromal cells, most notably adipocytes and fibroblasts (FIG. 22I,J), the two largest producers of neurotrophic growth factor (NGF) outside of the ENS in the data (Tables 19-22). Potential enteric neuron signaling to adipocytes spanned neuropeptides that regulate appetite and energy metabolism (CGRP/CALCRL, NPY/NPYR1) (55, 56), and two neurotransmitters (glutamate/GRM8, GABA/GABRE) (FIG. 22J). Adipocytes reciprocally signal to neurons via the leptin pathway, with all neuron subsets expressing the leptin receptor (LEPR) (FIG. 22J). In addition, inferred neuron signaling to fibroblasts included neuropeptides (PACAP/VIP/VIPR2) (FIG. 22J), neurotransmitters (glutamate/GRIA4, nitric oxide/GUCY1A3), growth factors (FGF1/FGFR1, PDGF/PDGFRB), guidance cues (SLIT2/ROBO1, SLIT3/ROBO2), and IL15/IL15R (FIG. 22J).

Even if cell subsets are not enriched for interactions, they may still interact through a more limited, but functionally important, receptor-ligand repertoire. Given recent reports describing neuro-immune crosstalk (1), Applicants searched for specific examples of interactions between neurons and immune cells (FIG. 22J). Applicants identified potential neuron signaling to (1) T cells via IL7/IL7R, IL12A/IL12RB1 (neuronal expression validated in situ, FIG. 22K,L), and PENK/OPRM1, (2) dendritic cells via CHAT/CHRNE, and (3) B cells via TPH2/HTR3A (FIG. 22J). Both IL-7 and IL-12 have key roles in lymphocyte and ILC survival and Th1 polarization (57), suggesting key pathways by which enteric neurons may regulate adaptive immunity. Finally, human PSN1s express NMU, which activates ILC2s (9); however, Applicants did not detect expression of the NMU receptor gene in the ILCs.

### Example 18

### Human Enteric Neurons Express Risk Genes for Enteric Neuropathies, Intestinal Inflammatory Disorders, and Extra-Intestinal Disorders with GI Dysmotility

To interrogate potential contributions of the ENS to human diseases, Applicants examined whether enteric neurons expressed any genes associated with diseases with varying degrees of known ENS involvement. These ranged from Hirschsprung's disease (HSCR), a primary enteroneuropathy that directly affects the ENS to autism spectrum disorder (ASD) and to Parkinson's disease (PD), which are extra-intestinal CNS disorders that are associated with dysfunctions in gut motility that occur early in disease progression (58-60). In addition, because the ENS is thought to play a pivotal role in inflammation—for example, through the activation of ILCs (9)—Applicants also examined whether IBD-associated genes are expressed by enteric neurons.

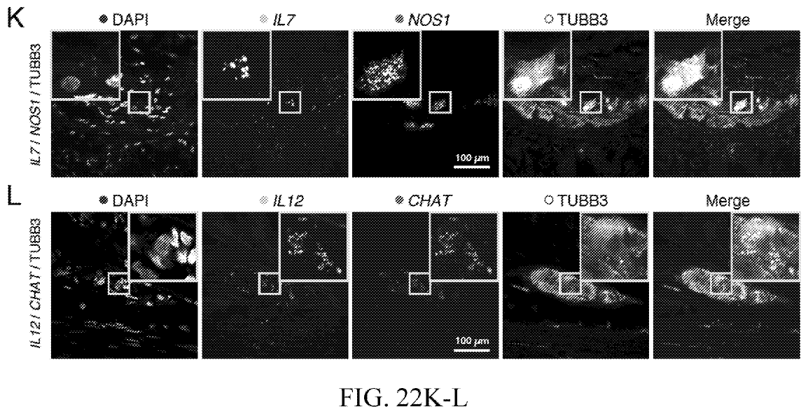

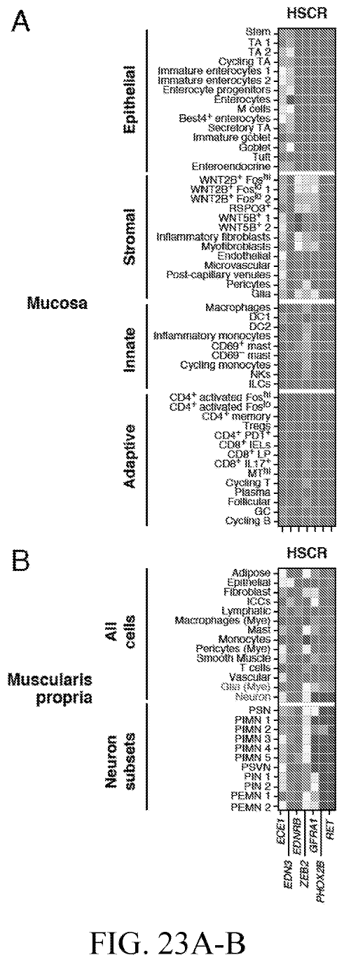

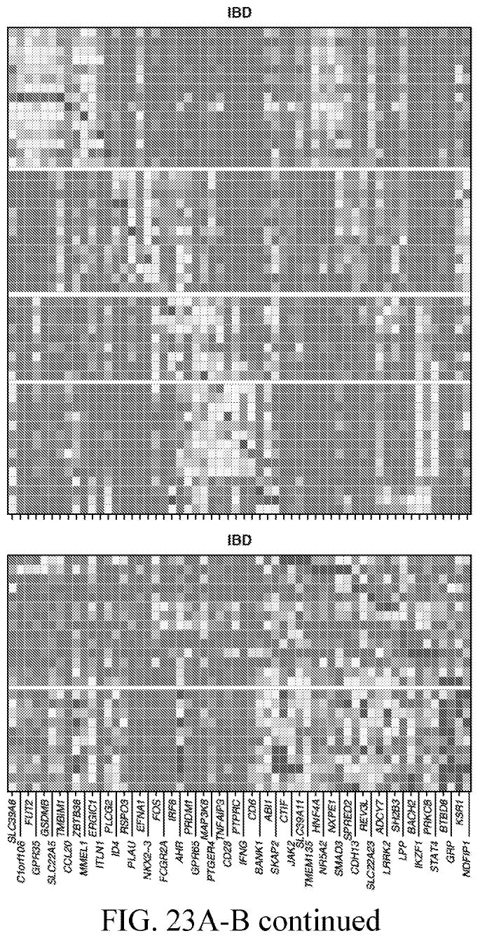

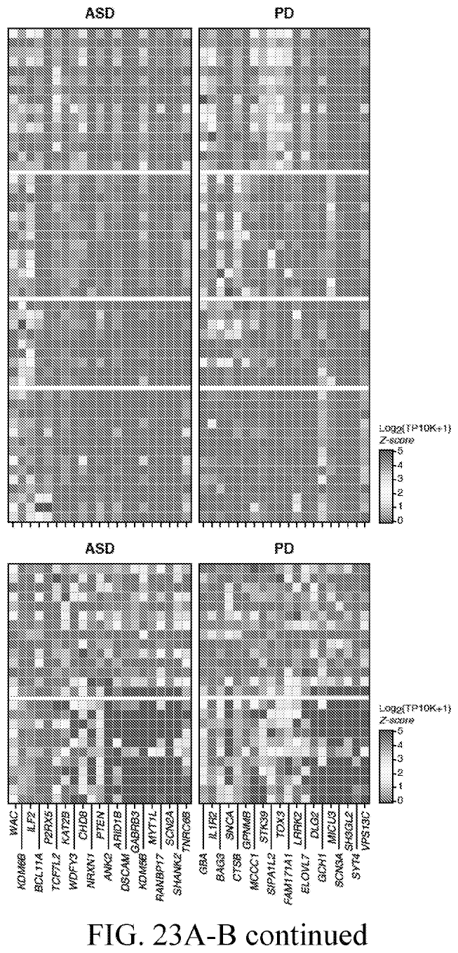

Mapping a curated list of 185 disease-associated genes (SOM) onto cell subsets from the muscularis propria, lamina propria, and epithelium (above), Applicants identified many genes that were specifically enriched in enteric neurons (FIG. 23A). For example, even though it is a neurodevelopmental disorder, Applicants mapped most HSCR-associated genes onto adult enteric neurons, including RET, PHOX2B, GFRA1, ZEB2, and ECE1 (FIG. 23A). The two exceptions, EDN3 and EDNRB, mediate endothelin signaling in the embryonic neural crest (61). Although most IBD risk genes localize to epithelial and immune cells, a subset of genes were most highly expressed in neurons, including GRP, BTBD8, KSR1, NDFIP1, and REV3L (FIG. 23B). In particular, GRP products stimulate GI hormone release, muscle contraction, and epithelial cell proliferation (62). Another such gene, REV3L, is also perturbed in the craniofacial neurologic disorder Möbius syndrome (63). Indeed, increased expression of many neuropeptides (e.g., tachykinin and galanin) has been reported in IBD patients (64).

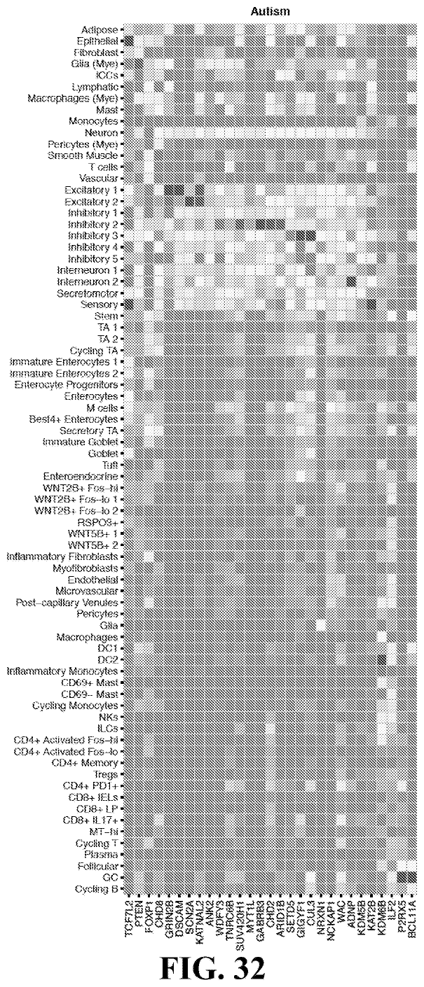

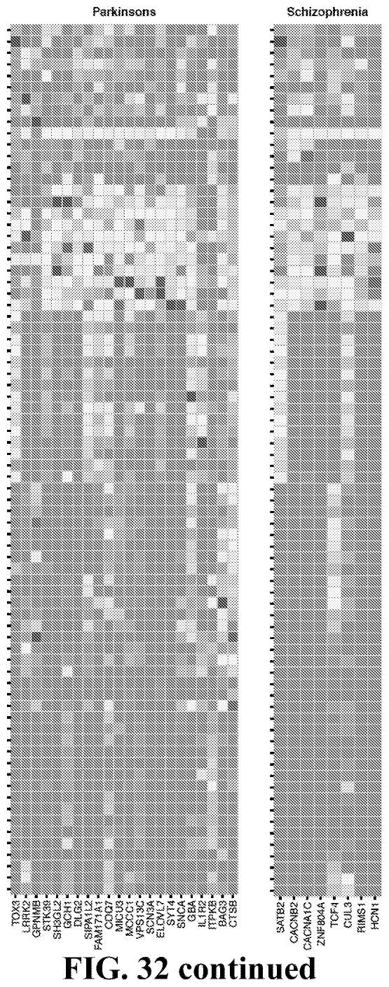

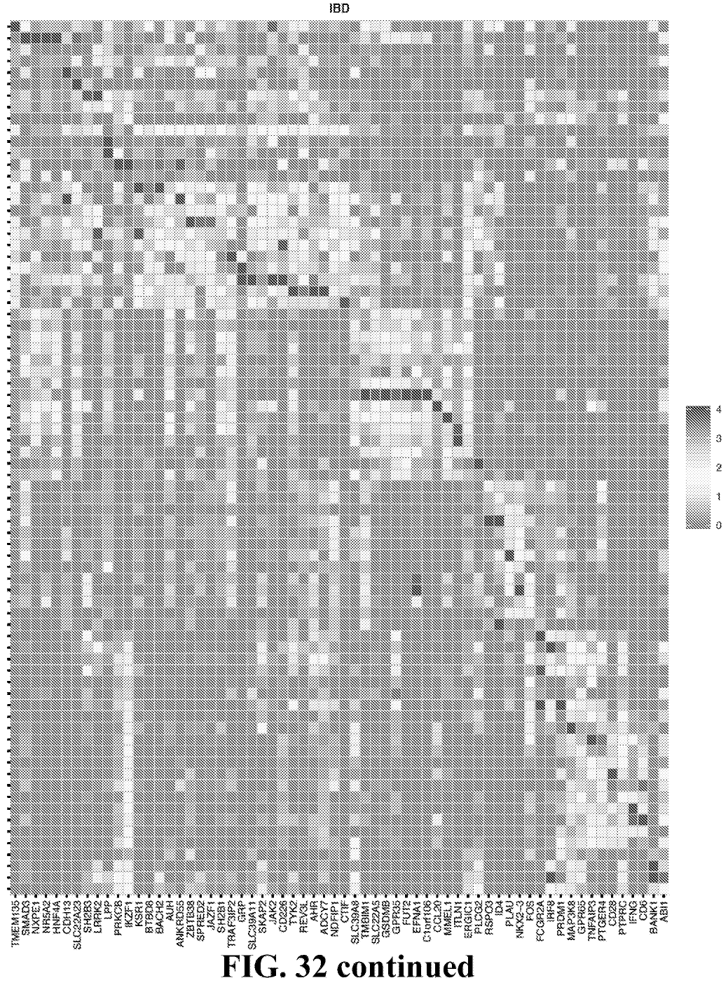

The risk genes for CNS diseases with concomitant GI dysfunction predominantly mapped to enteric neurons, with exceptions in ASD and PD (e.g., P2RX5 and IL1R2 in B cells and epithelial cells, respectively) (FIG. 23C). CNS disease risk genes that mapped specifically to enteric neurons include: (1) ANK2, DSCAM, and NRXN1 for ASD, and (2) DLG2, SCNA and SCN3A for PD (FIG. 23C). Expression of these risk genes specifically by enteric neurons, compared with a colon reference map, motivate further investigation of the role that enteric neurons play in the development and progression of dysmotility in intra- and extra-intestinal disorders. Applicants also show the disease risk genes for schizophrenia are expressed in neurons (FIG. 32).

### Example 19

### Discussion

Here, Applicants constructed reference maps of the colon enteric nervous system of adult mice and humans at single cell resolution, revealing the broad capacity of neurons to orchestrate tissue homeostasis. Isolating individual enteric neurons from adult animals for transcriptional profiling has not been previously possible due to technical limitations, and recent efforts using whole-cell dissociations have been limited to embryonic or post-natal animals (21, 22). The development of RAISIN and INNER Cell RNA-seq, which preserve ribosome-attached RNA on intact nuclei, allowed Applicants to profile 2,447 mouse and 831 human enteric neurons, along with other diverse cell types from both species (e.g., epithelial, stromal, and immune cells). These methods can be applied to both fresh and frozen tissue specimens, opening the way to characterizing the ENS and a range of archived frozen tissue samples. Additionally, preservation of the ER on nuclei may allow for the enrichment of nuclei with antibodies targeting specific membrane proteins, which are synthesized in the ER.

Applicants identified all major classes of enteric neurons, spanning 24 mouse subsets and 11 human subsets, including motor, sensory, secretomotor/vasodilator and interneuron types. Mining their expression signatures allowed Applicants to infer signaling among neurons and between neurons and non-neuronal cells, such as adipocytes, ICCs, immune cells, and epithelial cells. Applicants show circadian regulation of the ENS, including core clock genes, motivating further investigation into temporal variation of ENS function, nutrient absorption, and metabolism (65). Applicants also show differences in neuron composition across the mouse colon (e.g., sensory neurons enriched in the proximal colon) suggesting that ENS function varies along the length of the GI tract. Comparison of mouse and human neurons allowed Applicants to derive core transcriptional signatures for subsets across species, highlighting biological processes that can be modeled in mouse; for example, sensory neurons in both species express Noggin a gene known to support the epithelial stem cell niche (40). Taken together, these data enable the generation of testable hypothesis and experimental dissection of ENS function.

Finally, given the extensive neuro-immune signaling Applicants observe in the mouse and human ENS, Applicants propose that neuronal dysfunction can lead to immune dysregulation, which can exacerbate inflammation and related pathologies. For example, several IBD risk genes are expressed in neurons, raising the need to further characterize the role of enteric neurons in intestinal inflammation. Intriguingly, dozens of risk genes for early-life and late-onset CNS disorders with concomitant gut dysmotility are highly expressed by enteric neurons suggesting a mechanism for gut motility dysfunction in these diseases, and that profiling the much more accessible ENS may allow Applicants to study human disease biology. Furthermore, recent associations between the gut microbiota and extra-intestinal diseases, such as autoimmune disorders (reviewed in (66) and cancers and cancer therapies (reviewed in (67), suggest that immune modulation in the gut can have systemic effects. Proper immune function is thought to be necessary for CNS maintenance and repair, with immune dysregulation contributing to neurodegenerative disease (reviewed in (68). Thus, the ENS may be a central conduit linking the gut, the immune system and the brain, and neurological dysfunction in the gut may exacerbate diseases of the CNS.

### Example 20

### ENS Materials and Methods

Human donors and tissue samples. All colon resection samples were obtained from colon cancer patients after informed consent at either the Dana Farber Cancer Institute, Boston (IRB 03-189; ORSP 3490) or Massachusetts General Hospital, Boston (IRB 02-240; ORSP 1702). Metadata for the samples are provided in Tables 19-22. Normal colon located proximal to tumor was placed into conical tubes containing Roswell Park Memorial Institute (RPMI) media supplemented with 2% human serum and placed on ice for transport to the Broad Institute, Boston. Upon arrival, the muscularis propria was dissected from the remainder of the tissue (e.g., submucosa), divided into pieces (approximately 20-120 mg), which were placed into cryo-vials, frozen on dry-ice and stored at −80° C. When possible, a portion of the tissue was fixed overnight in 4% paraformaldehyde at 4° C. for histology.

Mouse models. All animal work was performed under the guidelines of the Division of Comparative Medicine, in accordance with the Institutional Animal Care and Use Committees (IACUC) relevant guidelines at the Broad Institute and MIT, and consistent with the Guide for Care and Use of Laboratory Animals, National Research Council, 1996 (institutional animal welfare assurance no. A4711-01), with protocol 0122-10-16. Mice were housed under specific-pathogen-free (SPF) conditions at the Broad Institute vivarium. The following strains were used:

TABLE 9

| | Jackson Laboratory | |
|---|---|---|
| | (Bar Harbor, ME) | |
| Strain | catalog number | Reference |
| C57BL/6J | 000664 | |
| B6; CBA-Tg(Sox10-cre)1Wdr/J | 025807 | (102) |
| 129S4.Cg-E2f1Tg(Wnt1-cre)2Sor/J | 022137 | (103) |
| B6; 129-Gt(ROSA)26Sortm5(CAG- | 021039 | (69) |
| Sun1/sfGFP)Nat/J | | |
| Tg(Uchl1-HIST2H2BE/mCherry/ | 016981 | (70) |
| EGFP*)FSout/J | | |

Tissue collection for snRNA-seq. For snRNA-seq optimization, tissue was collected from 11-14 week animals. For the ENS atlas, tissue was collected from 11-14 week old and 50-52 week old mice at either 7-8 am or 7-8 pm. Each colon was isolated and rinsed in ice cold PBS. Next, the colon was opened longitudinally and separated into four equally-sized sections, which were frozen in a 1.5 mL tube on dry ice. For brain collection, the brain was removed, quartered and frozen in a 1.5 mL tube on dry ice. Frozen tissue was stored at −80° C. until subsequent tissue processing.

Tissue collection and preparation for RNA fluorescence in situ hybridization and immunohistochemistry. For RNA fluorescence in situ hybridization (RNA FISH) and Immunohistochemistry (IHC), isolated colon was cut into four sections of equal size and processed as described (71). Briefly, tissue was fixed in 4% paraformaldehyde overnight at 4° C. Then, tissue was sequentially passed through PBS containing 7.5%, 15% and 30% (w/v) sucrose at 4° C. Tissue was then embedded in O.C.T. (23-730-571, Fisher Scientific, Hampton, N.H.) and stored at −80° C. Tissue was cut at 25 micron thick sections onto Superfrost Plus microscope slides (22-037-246, Fisher Scientific) using a Leica CM1950 Cryostat (Leica Biosystems Inc., Buffalo Grove, Ill.).

Immunofluorescence (IF). Slides with tissue sections were washed three times in PBS for 10 minutes, blocked 1 hour in CAS-Block Histochemical Reagent (00-8120, Thermo Fisher Scientific), incubated with primary antibodies overnight at 4° C., washed three times in PBS for 10 minutes, and then incubated with secondary antibodies at for 1 hour at room temperature. Slides were then washed twice in PBS for 10 minutes and then for 10 minutes with a PBS containing DAPI (D9542, Sigma-Aldrich). Lastly, slides were mounted using Southern Biotech Fluoromount-G (010001, VWR) and sealed. Antibodies used for IF: Rabbit anti-Tubb3 (1:1000, AB18207, Abcam), Chicken anti-mCherry (1:1000, AB356481, EMD Millipore), and Alexa Fluor 488-, 594-, and 647-conjugated secondary antibodies (Life Technologies) were used.

Single-molecule fluorescence in situ hybridization (smFISH). RNAScope Multiplex Fluorescent Kit (Advanced Cell Diagnostics) was used per manufacturer's recommendations for fresh-frozen samples with the following alterations. All Wash Buffer times were increased to 5 minutes and, following final HRP-Block step, slides were washed for 10 minutes with PBS containing DAPI (Sigma-Aldrich) followed by mounting with Southern Biotech Fluoromount-G (VWR) and sealed. Probes used for smISH (Advanced Cell Diagnostics): Calca (417961), Caleb (425511), Cck (402271), Chat (408731-C2), Grp (317861-C2), Nmu (446831), Nog (467391), Nos1 (437651-C3), Piezo1 (500511), Piezo2 (400191-C3), Sst (404631-C3), ANO1 (349021-C2), CHAT (450671 and 450671-C2), GUCY1A3 (425831), IL7 (424251), IL12A (402061), KIT (606401-C3), and NOS1 (506551-C2) were used.

Combined smFISH and IF. smFISH was performed as described above, with the following changes. After the final HRP-Block step, tissue sections were incubated with primary antibodies overnight at 4° C., washed in TBST for 5 minutes, twice, and then incubated with secondary antibodies for 30 min at room temperature. Slides were then washed in TBST for 5 minutes, twice, followed by a 10 minutes wash with containing DAPI (Sigma-Aldrich) before mounting with Southern Biotech Fluoromount-G (VWR) and sealed.

Confocal microscopy and image analysis. Images were taken using a Nikon TI-E microscope with a Yokohama W1 spinning disk, 405/488/561/640 lasers, and a Plan Apo 60×/1.4 objective. Images were visualized and overlaid using FIJI (72-75). The Bio-Formats plugin (76) was used to import all images.

Nuclei Extractions. The following nuclei extractions were performed from either mouse colon or brain and subsequently processed for profiling:

Dounce homogenization: Nuclei were extracted using either dounce homogenization followed by sucrose gradient centrifugation as described (77), or using the Nuclei EZ Prep (NUC101-1KT, Sigma-Aldrich) as described (78), with the following modifications. The tissues were dounce homogenized with a 7 mL Dounce Tissue Grinder (VWR 22877-280) (20 times pestle A, 20 times pestle B) and buffer volumes were increased to 5 mL for homogenization.

Tissue grinding: Fresh-Frozen tissues were crushed into a fine powder with a mortar and pestle (89038-144 and 89038-160, VWR) over a bath of liquid nitrogen. The powder was briefly resuspended in 2 mL of liquid nitrogen for transfer to a 50 mL conical tube, where liquid nitrogen was allowed to evaporate. The tissue powder was resuspended in 5 mL of Nuclei EZ Prep reagent (NUC101-1KT, Sigma-Aldrich) or NST (NP-40, Salts and Tris; see Tables 11 and 12) and transferred to a 7 mL Dounce Tissue Grinder. For the Nuclei EZ Prep kit, all subsequent steps were as described (78). For NST, the tissue was dounce homogenized with a 7 mL Dounce Tissue Grinder (VWR 22877-280) (20 times pestle A, 20 times pestle B), filtered through a 40 μm strainer (Falcon), and flow-through was spun at 500 g for 5 minutes at 4° C. The pellet was resuspended in 0.5-3 mL of ST (Salts: 146 mM NaCl, 1 mM CaCl2, 21 mM MgCl2; Tris; see Tables 11 and 12).

Chopping extraction: Fresh-frozen tissues were disaggregated in 1 mL of custom nuclear extraction buffer (see Tables 11 and 12 for all combinations used) with mild chopping by Tungsten Carbide Straight 11.5 cm Fine Scissors (14558-11, Fine Science Tools, Foster City, Calif.) for 10 minutes on ice. Large debris were removed with a 40 μm strainer (Falcon). An additional 1 mL of buffer was used to wash the filter before proceeding to fluorescence-activated cell sorting (FACS). For droplet-based RNA-Seq, nuclei were isolated as described above, but with the addition of 3 ml of ST (Salts and Tris; Tables 11 and 12) to extracted nuclei. Nuclei were then pelleted at 500 g for 5 mins at 4° C. Supernatant was discarded and the nuclei pellet was resuspended in 100-500 μL of ST buffer (Salts and Tris; Tables 11 and 12) before filtering through a 40 μm strainer-capped round bottom tube (Falcon).

Fluorescence-activated cell sorting (FACS). Prior to sorting, isolated nuclei and RAISINs were stained with Vybrant DyeCycle Ruby Stain (V-10309, Thermo Fisher Scientific). Sorting was performed on a MoFlo Astrios EQ Cell Sorter (Beckman Coulter) using 488 nm (GFP, 513/26 filter) or 561 nm (mCherry 614/20 filter), and 640 nm (Vybrant DyeCycle Ruby, 671/30 filter) lasers. Single nuclei were sorted into the wells of a 96-well PCR plate containing 5 μl of TCL buffer (1031576, Qiagen) with 1% β-mercaptoethanol. The 96 well plate was sealed tightly with a Microseal F and centrifuged at 800 g for 3 minutes before being frozen on dry ice. Frozen plates were stored at −80° C. until whole-transcriptome amplification, library construction, sequencing, and processing.

Whole-transcriptome amplification, library construction, sequencing, and processing. Libraries from isolated single nuclei and RAISINs were generated using SMART-seq2 as described (79), with the following modifications. RNA from individual wells was first purified with Agencourt RNAClean XP beads (A63987, Beckman Coulter) prior to oligo-dT primed reverse transcription with Maxima reverse transcriptase (EP0753, Thermo Fisher Scientific) and locked TSO oligonucleotide, which was followed by 21 cycles of PCR amplification using KAPA HiFi HotStart ReadyMix (NCO295239, KAPA Biosystems). cDNA was purified twice using Agencourt AMPure XP beads (A63881, Beckman Coulter) as described (79). The Nextera XT Library Prep kit (FC-131-1096, Illumina, San Diego, Calif.) with custom barcode adapters (sequences available upon request) was used for library preparation. Libraries from 384 wells (nuclei/RAISINs) with unique barcodes were combined and sequenced using a NextSeq 500 sequencer (FC-404-2005, Illumina).

Droplet-based RAISIN RNA-seq. Single RAISINs were processed through the GemCode Single Cell Platform using the GemCode Gel Bead kit (v2 chemistry), Chip and Library Kits (10× Genomics, Pleasanton, Calif.), following the manufacturer's protocol. RAISINs were resuspended in ST buffer (Salt and Tris; Tables 11 and 12). An input of 7,000 RAISINs was added to each channel of a chip. The RAISINs were then partitioned into Gel Beads in Emulsion (GEMs) in the GemCode instrument, where lysis and barcoded reverse transcription of RNA occurred, followed by amplification, shearing and 5′ adaptor and sample index attachment. Libraries were sequenced on an Illumina NextSeq 500.

Transmission electron microscopy (TEM). Extracted nuclei and RAISINs were pelleted and fixed at 4° C. overnight in 2.5% Glutaraldehyde and 2% Paraformaldehyde in 0.1 M sodium cacodylate buffer (pH 7.4). The pellet was then washed in 0.1M cacodylate buffer, and post-fixed with 1% Osmiumtetroxide (OsO4) and 1.5% Potassiumferrocyanide (KFeCN6) for 1 hour. Next, the pellet was washed in water 3 times and incubated in 1% aqueous uranyl acetate for 1 hour followed by 2 washes in water and subsequent dehydration in grades of alcohol (10 minutes each; 50%, 70%, 90%, 100%, and 100%). The pellet was then put in propyleneoxide for 1 hour and infiltrated overnight in a 1:1 mixture of propyleneoxide and TAAB Epon (Marivac Canada Inc. St. Laurent, Canada). The following day the samples were embedded in TAAB Epon and polymerized at 60° C. for 48 hours.

Ultrathin sections (about 60 nm) were cut on a Reichert Ultracut-S microtome, picked up on to copper grids stained with lead citrate and examined in a JEOL 1200EX Transmission electron microscope and images were recorded with an AMT 2k CCD camera.

Processing FASTQ reads into gene expression matrices. For SMART-seq2, FASTQ files were demultiplexed and aligned to a reference transcriptome (see “Mouse and human reference transcriptomes”), and transcripts were quantified using RSEM, as previously described (80). For droplet-based scRNA-Seq, Cell Ranger v2.0 was used to demultiplex the FASTQ reads, align them to a reference transcriptome, and extract their “cell” and “UMI” barcodes. The output of each pipeline is a digital gene expression (DGE) matrix for each sample, which records the number of transcripts or UMIs for each gene that are associated with each cell barcode. DGE matrices were filtered to remove low quality cells, defined as cells with fewer than 500 detected genes. This cutoff was set to remove contaminating cells, while retaining neurons and glia, which typically have high numbers of detected genes. To account for differences in sequencing depth across cells, DGE counts were normalized by the total number of transcripts or UMIs per cell and converted to transcripts-per-10,000 (henceforth “TP10K”).

Mouse and human reference transcriptomes. For the optimization of nuclei extraction conditions, reads were aligned to the mm10 reference transcriptome. However, for the mouse and human ENS atlases, Applicants augmented the reference transcriptomes with introns, thus allowing pre-mRNAs to be mapped along with mature mRNAs. Both the mm10 and hg19 reference transcriptomes were modified according to the instructions provided by the 10× Genomics web site (support. 10×genomics.com/single-cell-gene-expression/software/pipelines/latest/advanced/references). Briefly, Applicants converted the standard GTF files into pre-mRNA GTF files by changing all “transcript” feature tags to “exon” feature tags. Using these modified GTF files, Applicants then constructed Cell Ranger compatible references using the Cell Ranger “mkref” command. These modified GTF files were used for both the Cell Ranger pipeline and for the SMART-seq2 data (i.e. mouse ENS atlas).

Cell clustering overview. To cluster single cells into distinct cell subsets, Applicants followed the general procedure Applicants have previously outlined in (81) with additional modifications. This workflow includes the following steps: the selection of variable genes, batch correction, dimensionality reduction by PCA, and clustering. In all cases, clustering was performed twice: first, to separate neurons and glia from other cells, and then, to sub-cluster the neurons and glia to obtain high-resolution clusters within each group.

Partitioning cells into neuron, glia, and “other” compartments. Cells were partitioned into neuron, glia, and non-ENS compartments based on their expression of known marker genes (see “Gene signatures”). Signature scores were calculated as the mean log 2(TP10K+1) across all genes in the signature. Each cluster was assigned to the compartment of its maximal score and all cluster assignments were inspected to ensure the accurate segregation of cells. Neurons and glia were then assembled into two separate DGE matrices for further analysis.

Variable gene selection. To identify variable genes within a sample, Applicants first calculated the mean (μ) and the coefficient of variation (CV) of expression of each gene. Genes were then grouped into 20 equal-frequency bins (ventiles) according to their mean expression levels. LOESS regression was used to fit the relationship, log(CV)˜log(μ), and the 1,500 genes with the highest residuals were evenly sampled across these expression bins. To extend this approach to multiple samples, Applicants performed variable gene selection separately for each sample to prevent “batch” differences between samples from unduly impacting the variable gene set. A consensus list of 1,500 variable genes was then formed by selecting the genes with the greatest recovery rates across samples, with ties broken by random sampling. This consensus gene set was then pruned through the removal of all ribosomal, mitochondrial, immunoglobulin, and HLA genes, which were found to induce unwanted batch effects in some samples in downstream clustering steps.

Batch correction. Applicants observed substantial variability between cells that had been obtained from different mice or different individuals, which likely reflects a combination of technical and biological differences. In some cases, these “batch effects” led to cells clustering first by mouse or individual, rather than by cell type or cell state. To control for these batch differences, Applicants ran ComBat (Johnson et al., 2007) with default parameters on the log 2(TP10K+1) expression matrix, allowing cells to be clustered by cell type or cell state. Importantly, these batch-corrected data were only used for the PCA and other steps relying on PCA (e.g. clustering, t-SNE visualization); all other analyses (e.g. differential expression analysis) were based on the original expression data. Note that Applicants tested two additional methods for batch correction—one based on Canonical Correlation Analysis (82) and another on a k-nearest neighbors (k-NN) approach (79)—but did not obtain any enhancement in performance (data not shown).

Dimensionality reduction, graph clustering, and t-SNE visualization. Cells were clustered at two stages of the analysis: first, to initially partition the cells into neuron, glia, and “other” compartments, and second, to sub-cluster neurons and glia into different subsets. In all cases, Applicants ran low-rank PCA on the variable genes of the batch-corrected log 2(TP10K+1) expression matrix. Applicants then applied Phenograph (Levine et al., 2015) to the k-NN graph defined using the first n PCs and k nearest neighbors, which were separately estimated for each dataset. First, to estimate n, Applicants calculated the number of “significant” PCs using a permutation test. Because this test may underestimate the number of PCs, Applicants conservatively increased this number (i.e. to 15 or 30; see Table 10 below) to ensure that most of the variability in the dataset was captured. Next, to estimate k, Applicants considered a range of clustering solutions with varying values of k, and calculated the marker genes for each set of clusters. Applicants selected k based on inspection of the data. When clustering data from multiple cell types, Applicants tried to select k such that the major cell types (e.g. neurons, glia, and muscle) were split, without fragmenting them into several sub-clusters. When clustering neurons and glia, Applicants tried to select a k yielding the highest granularity clusters that were still biologically distinct, determined by close examination of the marker gene lists. Finally, the Barnes-Hut t-Distributed Stochastic Neighbor Embedding (t-SNE) algorithm was run on the selected PCs with perplexity=20 and for 10,000 iterations to produce two-dimensional embeddings of the data for visualization.

TABLE 10

| | Cell | # Sig | Used | |
|---|---|---|---|---|
| Dataset | type | PCs | PCs | k-NN |
| Optimization | All cells | 13 | 1 to 15 | 250 (separates neurons and glia) |
| Mouse atlas | All cells | 16 | 1 to 30 | 250 (separates neurons and glia) |
| Mouse atlas | Neurons | 15 | 1 to 30 | 25 |
| Mouse atlas | Glia | 7 | 1 to 15 | 250 |
| Mouse | All cells | 19 | 1 to 30 | 100 (separates major cell types) |
| droplet | | | | |
| Human atlas | All cells | 20 | 2 to 30* | 100 (separates major cell types) |
| Human atlas | Neurons | 9 | 1 to 15 | 25 |
| Human atlas | Glia | 8 | 1 to 15 | 100 |

*See “Clustering of human neurons”.

Clustering of human neurons. Initial clustering of the 831 human neurons revealed 15 subsets (FIG. 30H). However, in several cases, Applicants noticed that a single neuron type had been split into two clusters based on the expression of oxidative phosphorylation genes, which were strongly enriched in PCI (FIG. 30I,J). This could reflect differences in differentiating vs. mature neurons (79), cancer-proximal effects, or a rapid transcriptional response to tissue resection or handling. Applicants therefore re-clustered the cells based on the other PCs (i.e. PCs 2 to 30), yielding 11 final subsets of human neurons (FIG. 30C,G).

Scoring nuclei extraction conditions. To identify optimal conditions for snRNA-seq of the ENS, Applicants performed nuclei extractions while systematically varying the detergent (CHAPS, Digitonin, EZ, NP40, Tween), buffer (HEPES, Tricine, Tris), mechanical extraction conditions (Dounce, Grind, Chop), and additional modifiers (e.g. polyamines, RNAse inhibitors) (Tables 11 and 12). In total, 104 different extraction conditions were examined. For each extraction, Applicants profiled single nuclei transcriptomes by SMART-Seq2 and clustered the resulting RNA into neurons, glia, and “other” (i.e. non-ENS or low quality) clusters (see “Cell clustering overview”). To compare extractions, Applicants calculated several quality metrics for each condition: (1) the proportion of recovered neurons, glia, and “other” cells, (2) the mean number of detected genes per cell, and (3) the mean ENS signature score (derived from markers of neurons and glia; see “Cell type signatures”). Conditions that yielded high-quality nuclei enriched in the ENS signature score were then identified.

Cell lineage dendrogram. As an auxiliary tool, cell subsets were organized on a dendrogram according to their transcriptional similarities (FIG. 21B, top). To construct this tree, Applicants performed complete linkage clustering on the distance matrix corresponding to the mean transcriptional distances among all cell subsets, calculated using the variable genes from the log 2(TP10K+1) expression matrix. These calculations were performed using the “hclust” and “dist” functions in R with default parameters.

Enteric neuron annotation and classification. Applicants employed the following markers and considerations in annotating enteric neurons subsets post hoc.

### Broad Segmentation of the Mouse ENS

Broadly, neurons segmented into two major divisions comprising either cholinergic or nitrergic subsets. This broad division was correlated with several other genes. For example, the glial cell line-derived neurotrophic factor (GDNF) family receptors α1 (Gfra1) and α2 (Gfra2) segregate Nos1 and Chat expressing neurons, respectively. Gfra1/2 are co-receptors for the GDNF receptor, Ret, which is necessary for ENS formation (83,84). Similar, Chat and Nos1 expressing subsets also differentially expressed the transcription factors (TFs), Casz1 and Etv1.

### Annotating Mouse Excitatory Motor Neurons

Applicants annotated 6 subsets of putative excitatory motor neurons (PEMNs) based on co-expression of Chat and Tac1 (85) and position within the dendrogram on one subtree (FIG. 21B). Subsets of PEMNs express the endogenous opioid, enkephalin (Penk), which is found in motor neurons (85), and/or the myenteric motor neuron marker, calretinin (Calb2) (86).

### Annotating Mouse Inhibitory Motor Neurons

Applicants annotated 7 subsets of putative inhibitory motor neurons (PIMNs), which have high Nos1 and Vip co-expression (87,88), and occupy one subtree of the dendrogram (FIG. 21B). In total, 73% of Vip-positive neurons co-express Nos1, which is consistent with the previously reported estimate of 75% (87,88). In addition, PIMN 6 and 7 have significant expression of somatostatin receptor 2 (Sstr2), which plays an important role in cauded relaxation, as blocking Sstr2 nearly abolishes muscle relaxation (87).

### Annotating Mouse Interneurons

Enteric interneurons (INs) relay sensory information and coordinate excitatory and inhibitory motor neuron activity, but their classification is unclear. Six potential subtypes have been previously reported: (1) descending INs that signal via Chat, 5HT and ATP, (2) descending Nos1+Vip+Grp+Chat− INs, (3) descending Vip+Chat+Nos1+ INs with ATP signaling, (4) descending Chat+Sst+ INs, (5) descending Penk+ INs (responsive to Sst), and (6) ascending Chat+Penk+ INs with ATP signaling (87, 89-91).

Some of these subsets (3, 5, 6) are at least partly matched as discrete clusters in the data, whereas others (1, 2, 4) are not clearly observed in the atlas. PIMN7 is a potential candidate for the descending Vip+Chat+Nos1+ INs with ATP signaling (3 above), based on co-expression of Vip, Chat, Nos1, and various ATP transporters (e.g. SLC28a1, Slc28a2, Slc28a3, Slc29a1, Slc29a2, Slc29a3, Slc29a4; (85). PSN3 also express these genes, but their expression of Cck, Calca, and Calcb makes it unlikely they are interneurons. Three subsets of Chat+Penk+ putative INs (PIN1-3) may reflect either descending Penk+ INs (5 above; responsive to Sst), or ascending Chat+Penk+ INs with ATP signaling (6 above). Because all express combinations of Sst receptors, they may be descending INs. However, given the substantial number of additional receptors expressed by all of these PINs (for SHT, VIP, GAL, GLP, prolactin, prostaglandin E2, EGF and BMP) or some of them (e.g., catecholamine synthetic enzymes), they may not be INs. Finally, there was little to no evidence for other IN subtypes: Applicants did not detect any serotonergic (5HT) neurons (1 above) in the sampling, consistent with previous observations (88); found no discernible cluster of Nos1+Vip+Grp+Chat− cells; and the only Chat+Sst+ neurons Applicants observed were the Calcb+ PSN4 subset, which Applicants interpret as a sensory neuron, not INs.

### Annotating Mouse Secretomotor and Vasodilator Neurons

Applicants annotated two subsets of Glp2r+ putative secretomotor/vasodilator (PSVNs) in one subtree of the dendrogram (FIG. 21B), one Vip+ non-cholinergic subtype (PSVN1) and one Chat+ cholinergic subset (PSVN2). The PSVN2 subset expresses Gal, previously reported in neurons that innervate the epithelium and arterioles (92) and neuropeptide Y expressed in a secretomotor neurons (90). Also, some neurons in PSVN2 expresses glutamate decarboxylase 2 (Gad2), possibly forming cholinergic/GABAergic neurons.

### Annotating Human Interneuron Subtype 2

Human PIN2s express two specific markers of mouse sensory neurons, CALCB and GRP, suggesting they may be misannotated sensory neurons. Another possibility is that PIN2s correspond to multiple neuron subtypes, which cannot be resolved with the number of neurons Applicants profiled. Consistent with this possibility, PENK and CALCB expression are mutually exclusive within this subset (3 of 34 co-positive cells; expected=7.24; Fisher test, p<0.001).

Differential expression analysis. Differential expression (DE) tests were performed using MAST (Finak et al., 2015), which fits a hurdle model to the expression of each gene, consisting of logistic regression for the zero process (i.e. whether the gene is expressed) and linear regression for the continuous process (i.e. the expression level). For the mouse atlas, this regression model included terms to capture the effects of the cell subset, age, sex, colon location, circadian phase, transgenic model, and cell complexity. For the human atlas, this regression model only included terms for cell subset and cell complexity.

For the mouse atlas, Applicants used the regression formula, Yi˜X+A+C+L+S+T+N, where Yi is the standardized log 2(TP10K+1) expression vector for gene i across cells, X is a variable reflecting cell subset membership (e.g. PSNs vs. non-PSNs), A is the age associated with each cell (adult vs. aged), C is the circadian phase for each cell (morning vs. evening), L is the location for each cell (segments 1-4), S is the sex for each cell (male vs. female), T is the transgenic model for each cell (Sox10 vs. Uchl1), and N is the standardized number of genes for each cell (i.e. cell complexity). For the human atlas, Applicants used the regression formula, Yi˜X+N, with X and N defined as above.

Additionally, two heuristics were used to increase the speed of the tests: Applicants required all tested genes to have a minimum fold change of 1.2 and to be expressed by at least 1% of the cells within the group of interest. In all cases, the discrete and continuous coefficients of the model were retrieved and p-values were calculated using the likelihood ratio test in MAST. Q-values were separately estimated for each cell subset comparison using the Benjamini-Hochberg correction. Unless otherwise indicated, all reported DE coefficients and q-values correspond to the discrete component of the model (i.e. the logistic regression).

Acquisition and scoring of gene signatures. Applicants compiled the following lists of marker genes for enteric neurons and glia from the literature (93). These gene signatures were then combined to construct an overall “ENS” signature score (FIG. 20C and FIG. 25).

Neurons: Tubb3, Elavl4, Ret, Phox2b, Chrnb4, Eml5, Smpd3, Tagln3, Snap25, Gpr22, Gdap1l1, Stmn3, Chrna3, Scg3, Syt4, Ncan, Crmp1, Adcyap1r1, Elavl3, Dlg2, Cacna2d.

Glia: Erbb3, Sox10, Fabp7, Plp1, Gas7, Nid1, Qk, Sparc, Mest, Nfia, Wwtr1, Gpm6b, Rasa3, Flrt1, Itpripl1, Itga4, Lama4, Postn, Ptprz1, Pdpn, Col18a1, Nrcam.

To prevent highly expressed genes from dominating a gene signature score, Applicants scaled each gene vector of the log 2(TP10K+1) expression matrix by its root mean squared expression across all cells (using the ‘scale’ function in R with center=FALSE). The signature score for each cell was then computed as the mean scaled expression across all genes in the signature.

Estimation of false discovery rate. Unless otherwise specified, false discovery rates were estimated with the Benjamini-Hochberg correction (94), using the “p.adjust” R function with the “fdr” method.

Matching human and mouse subsets. To map human neurons onto their mouse counterparts, Applicants first trained a Random Forest classifier to distinguish the each of 24 subsets of mouse neurons (i.e., PEMN, PIMN, PIN, PSN, PSVN) using the log 2(TP10K+1) expression matrix of the mouse variable genes that also had human orthologs (see “Variable gene selection”). The Random Forest model was built with the R “randomForest” package using default parameters with the following exception: to account for class imbalances, Applicants down-sampled each neuron class to the minimum class size while constructing each tree (implemented using the “sampsize” argument). In total, the “out of bag” estimate of the error rate (which estimates test rather than training error) was 8.8%, indicating that Applicants can accurately distinguish among major neuron classes. Next, to extend this model to humans, Applicants predicted the class for each human neuron using expression data for the human orthologs of the variable genes. All class assignments were then manually examined to ensure accurate predictions for all cells. Note that Applicants also tested an alternative approach using a variational autoencoder (VAE) (95), but did not observe a noticeable improvement in performance (data not shown).

Identifying a core transcriptional program for major neuron classes. To identify conserved transcriptional signatures for each of the 5 major neuron classes (i.e., PEMN, PIMN, PIN, PSN, PSVN), Applicants first mapped all mouse genes to their corresponding human orthologs (using only 1:1 orthologs), and combined both expression matrices according to these genes. Applicants next calculated DE orthologs within each major neuron class (see “Differential expression analysis”), then selected genes that were significantly DE in the combined dataset, the mouse dataset, and the human dataset (Table 6).

Using receptor-ligand pairs to infer cell-cell interactions. To identify cell-cell interactions, Applicants mapped the FANTOM5 database of literature-supported receptor-ligand interactions (96) onto the lists of cell subset markers. Following a recent approach (CellPhoneDB (97)), Applicants filtered this database to remove all integrins (defined using the HUGO “Integrin” gene group), which were involved in many non-specific cell-cell interactions. Applicants further required cell subset markers to be expressed in at least 5% of all cells within the subset. For all networks, Applicants quantified the interaction strength between two cell subsets as the number of unique receptors and ligands connecting them, resulting in an adjacency matrix summarizing all cell-cell interactions within the dataset. Statistical significance was then empirically assessed by permuting the receptors and ligands among all cell subsets, thus preserving the number of receptors and ligands encoded within each cell subset, and preserving the distribution of ligand-receptor connectivity (but possibly changing the connectivity between cell subsets, in those cases where one receptor has multiple ligands, or vice versa). After running 10,000 total permutations, p-values were computed as the number of times the edge strength in the permuted network was greater than or equal to the edge strength in the true network. To plot cell-cell interaction networks, Applicants applied the Fruchterman-Reingold layout algorithm to a network defined using the −log 10(p-value), using only the edges with p-value<0.05. Although edge weights were used to generate the layout, they were removed from the final visualization for visual clarity (FIG. 22I).

Defining disease risk genes. Applicants compiled lists of genes that have been implicated by human genetics or genome-wide association studies (GWAS) as contributing to risk for the following diseases: Hirschsprung's disease (HRSC), inflammatory bowel disease (IBD), autism spectrum disorders (ASDs), and Parkinson's disease (PD). Because GWAS or human genetics studies do not always pinpoint a causative risk gene, Applicants used the literature to identify sets of genes that are particularly likely to contribute to disease risk, including: 9 HRSC-associated genes (98), 106 IBD-associated genes (99), 28 ASD-associated genes (100), and 29 PD-associated genes (101).

### Tables

Tables 11-12. Optimization of nuclei extractions for the enteric nervous system. Description and statistics for nuclei extractions, aggregated either by sample (Table 11) or condition (Table 12). Includes descriptions of the buffers, detergents, detergent concentrations, salts, and modifiers profiled, along with various statistics, including exon:intron ratios, the number of genes per cell, and ENS compositions.

TABLE 11
Samples

| Sample | Extraction | | | | | | Detergent | |
|---|---|---|---|---|---|---|---|---|
| ID | solution | Tissue | Preparation | Buffer | Salt | Detergent | Concentration | Modifier |
| S1 | NST | Colon | chop | Tris | 146 mM NaCl, 1 mM | NP40 | 0.2 | N/A |
| | | | | | CaCl2, 21 mM MgCl2 | | | |
| S2 | NST | Colon | chop | Tris | 146 mM NaCl, 1 mM | NP40 | 0.05 | N/A |
| | | | | | CaCl2, 21 mM MgCl2 | | | |
| S3 | NST | Colon | chop | Tris | None | None | 0 | N/A |
| S4 | NST | Colon | chop | Tricine | 146 mM NaCl, 1 mM | NP40 | 0.2 | N/A |
| | | | | | CaCl2, 21 mM MgCl2 | | | |
| S5 | NST | Colon | chop | Tris | 146 mM NaCl, 1 mM | NP40 | 0.2 | Polyamines |
| | | | | | CaCl2, 21 mM MgCl2 | | | |
| S6 | NST | Colon | chop | Tricine | 146 mM NaCl, 1 mM | NP40 | 0.2 | Polyamines |
| | | | | | CaCl2, 21 mM MgCl2 | | | |
| S7 | NST | Colon | chop | Tricine | 146 mM NaCl, 1 mM | NP40 | 0.2 | N/A |
| | | | | | CaCl2, 21 mM MgCl2 | | | |
| S8 | NST | Colon | chop | Tris | 146 mM NaCl, 1 mM | NP40 | 0.2 | N/A |
| | | | | | CaCl2, 21 mM MgCl2 | | | |
| S9 | NST | Colon | chop | Tris | 146 mM NaCl, 1 mM | NP40 | 0.001 | N/A |
| | | | | | CaCl2, 21 mM MgCl2 | | | |
| S10 | DST | Colon | chop | Tris | 146 mM NaCl, 1 mM | Digitonin | 0.01 | N/A |
| | | | | | CaCl2, 21 mM MgCl2 | | | |
| S11 | TST | Colon | chop | Tris | 146 mM NaCl, 1 mM | Tween | 0.006 | N/A |
| | | | | | CaCl2, 21 mM MgCl2 | | | |
| S12 | TST | Colon | chop | Tris | 146 mM NaCl, 1 mM | Tween | 0.0012 | N/A |
| | | | | | CaCl2, 21 mM MgCl2 | | | |
| S13 | TST | Colon | chop | Tris | 146 mM NaCl, 1 mM | Tween | 0.03 | N/A |
| | | | | | CaCl2, 21 mM MgCl2 | | | |
| S14 | NST | Colon | chop | Tris | 146 mM NaCl, 1 mM | NP40 | 0.2 | Sucrose |
| | | | | | CaCl2, 21 mM MgCl2 | | | |
| S15 | NST | Colon | chop | Tris | 146 mM NaCl, 1 mM | NP40 | 0.2 | N/A |
| | | | | | CaCl2, 21 mM MgCl2 | | | |
| S16 | NST | Colon | chop | Tris | 146 mM NaCl, 1 mM | NP40 | 0.025 | N/A |
| | | | | | CaCl2, 21 mM MgCl2 | | | |
| S17 | NST | Colon | chop | Tris | 146 mM NaCl, 1 mM | NP40 | 0.005 | N/A |
| | | | | | CaCl2, 21 mM MgCl2 | | | |
| S18 | TST | Colon | chop | Tris | 146 mM NaCl, 1 mM | Tween | 0.003 | N/A |
| | | | | | CaCl2, 21 mM MgCl2 | | | |
| S19 | TST | Colon | chop | Tris | 146 mM NaCl, 1 mM | Tween | 0.0012 | N/A |
| | | | | | CaCl2, 21 mM MgCl2 | | | |
| S20 | TST | Colon | chop | Tris | 146 mM NaCl, 1 mM | Tween | 0.00024 | N/A |
| | | | | | CaCl2, 21 mM MgCl2 | | | |
| S21 | DST | Colon | chop | Tris | 146 mM NaCl, 1 mM | Digitonin | 0.002 | N/A |
| | | | | | CaCl2, 21 mM MgCl2 | | | |
| S22 | DST | Colon | chop | Tris | 146 mM NaCl, 1 mM | Digitonin | 0.01 | N/A |
| | | | | | CaCl2, 21 mM MgCl2 | | | |
| S23 | DST | Colon | chop | Tris | 146 mM NaCl, 1 mM | Digitonin | 0.05 | N/A |
| | | | | | CaCl2, 21 mM MgCl2 | | | |
| S24 | CST | Colon | chop | Tris | 146 mM NaCl, 1 mM | CHAPS | 0.49 | N/A |
| | | | | | CaCl2, 21 mM MgCl2 | | | |
| S25 | CST | Colon | chop | Tris | 146 mM NaCl, 1 mM | CHAPS | 0.098 | N/A |
| | | | | | CaCl2, 21 mM MgCl2 | | | |
| S26 | CST | Colon | chop | Tris | 146 mM NaCl, 1 mM | CHAPS | 0.0196 | N/A |
| | | | | | CaCl2, 21 mM MgCl2 | | | |
| S27 | TST | Brain | chop | Tris | 146 mM NaCl, 1 mM | Tween | 0.0012 | N/A |
| | | | | | CaCl2, 21 mM MgCl2 | | | |
| S28 | NP40 | Brain | chop | Tris | 146 mM NaCl, 1 mM | NP40 | 0.2 | N/A |
| | | | | | CaCl2, 21 mM MgCl2 | | | |
| S29 | Sigma-Aldrich | Brain | dounce | EZ | N/A | | N/A | N/A |
| | EZ prep | | | | | | | |
| S30 | TST | Colon | chop | Tris | 146 mM NaCl, 1 mM | Tween | 0.0012 | N/A |
| | | | | | CaCl2, 21 mM MgCl2 | | | |
| S31 | NST | Colon | chop | Tris | 146 mM NaCl, 1 mM | NP40 | 0.2 | N/A |
| | | | | | CaCl2, 21 mM MgCl2 | | | |
| S32 | Sigma-Aldrich | Colon | grind | EZ | | EZ | #N/A | N/A |
| | EZ prep | | | | | | | |
| S33 | TST | Colon | chop | Tris | 146 mM NaCl, 1 mM | Tween | 0.0012 | N/A |
| | | | | | CaCl2, 21 mM MgCl2 | | | |
| S34 | TSH | Colon | chop | HEPES | 146 mM NaCl, 1 mM | Tween | 0.0012 | N/A |
| | | | | | CaCl2, 21 mM MgCl2 | | | |
| S35 | TST | Colon | chop | Tris | 146 mM NaCl, 1 mM | Tween | 0.0012 | Protease inhibitor |
| | | | | | CaCl2, 21 mM MgCl2 | | | |
| S36 | TST | Colon | chop | Tris | 146 mM NaCl, 1 mM | Tween | 0.0012 | Translation inhibitor |
| | | | | | CaCl2, 21 mM MgCl2 | | | |
| S37 | TST | Colon | chop | Tris | 146 mM NaCl, 1 mM | Tween | 0.0012 | Cytoskeletal drug |
| | | | | | CaCl2, 21 mM MgCl2 | | | |
| S38 | TST | Colon | chop | Tris | 146 mM NaCl, 1 mM | Tween | 0.0012 | Rnase inhibitor |
| | | | | | CaCl2, 21 mM MgCl2 | | | |
| S39 | NSH | Colon | chop | HEPES | 146 mM NaCl, 1 mM | NP40 | 0.2 | N/A |
| | | | | | CaCl2, 21 mM MgCl2 | | | |
| S40 | DSH | Colon | chop | HEPES | 146 mM NaCl, 1 mM | Digitonin | 0.01 | N/A |
| | | | | | CaCl2, 21 mM MgCl2 | | | |
| S41 | DST | Colon | chop | Tris | 146 mM NaCl, 1 mM | Digitonin | 0.01 | N/A |
| | | | | | CaCl2, 21 mM MgCl2 | | | |
| S42 | NSH | Colon | chop | HEPES | 146 mM NaCl, 1 mM | NP40 | 0.2 | N/A |
| | | | | | CaCl2, 21 mM MgCl2 | | | |
| S43 | NST | Colon | chop | Tris | 146 mM NaCl, 1 mM | NP40 | 0.2 | N/A |
| | | | | | CaCl2, 21 mM MgCl2 | | | |
| S44 | NST | Colon | chop | Tris | 146 mM NaCl, 1 mM | NP40 | 0.01 | N/A |
| | | | | | CaCl2, 21 mM MgCl2 | | | |
| S45 | TSH | Colon | chop | HEPES | 146 mM NaCl, 1 mM | Tween | 0.0012 | N/A |
| | | | | | CaCl2, 21 mM MgCl2 | | | |
| S46 | TST | Colon | chop | Tris | 146 mM NaCl, 1 mM | Tween | 0.0012 | N/A |
| | | | | | CaCl2, 21 mM MgCl2 | | | |
| S47 | TSH | Colon | chop | HEPES | 146 mM NaCl, 1 mM | Tween | 0.006 | N/A |
| | | | | | CaCl2, 21 mM MgCl2 | | | |
| S48 | TST | Colon | chop | Tris | 146 mM NaCl, 1 mM | Tween | 0.006 | N/A |
| | | | | | CaCl2, 21 mM MgCl2 | | | |
| S49 | NSH | Colon | chop | HEPES | 146 mM NaCl, 1 mM | NP40 | 0.01 | N/A |
| | | | | | CaCl2, 21 mM MgCl2 | | | |
| S50 | TSH | Colon | chop | HEPES | 146 mM NaCl, 1 mM | Tween | 0.0012 | N/A |
| | | | | | CaCl2, 21 mM MgCl2 | | | |
| S51 | Sigma-Aldrich | Colon | grind | EZ | N/A | EZ | #N/A | N/A |
| | EZ prep | | | | | | | |
| S52 | Sigma-Aldrich | Colon | grind | EZ | N/A | EZ | #N/A | N/A |
| | EZ prep | | | | | | | |
| S53 | TST | Colon | chop | Tris | 146 mM NaCl, 1 mM | Tween | 0.006 | N/A |
| | | | | | CaCl2, 21 mM MgCl2 | | | |
| S54 | CST | Colon | chop | Tris | 146 mM NaCl, 1 mM | CHAPS | 0.49 | N/A |
| | | | | | CaCl2, 21 mM MgCl2 | | | |
| S55 | CST | Colon | chop | Tris | 146 mM NaCl, 1 mM | CHAPS | 0.098 | N/A |
| | | | | | CaCl2, 21 mM MgCl2 | | | |
| S56 | CST | Colon | chop | Tris | 146 mM NaCl, 1 mM | CHAPS | 0.0196 | N/A |
| | | | | | CaCl2, 21 mM MgCl2 | | | |
| S57 | CST | Colon | chop | Tris | 146 mM NaCl, 1 mM | CHAPS | 0.49 | N/A |
| | | | | | CaCl2, 21 mM MgCl2 | | | |
| S58 | CST | Colon | chop | Tris | 146 mM NaCl, 1 mM | CHAPS | 0.098 | N/A |
| | | | | | CaCl2, 21 mM MgCl2 | | | |
| S59 | CST | Colon | chop | Tris | 146 mM NaCl, 1 mM | CHAPS | 0.0196 | N/A |
| | | | | | CaCl2, 21 mM MgCl2 | | | |
| S60 | TST | Colon | chop | Tris | 146 mM NaCl, 1 mM | Tween | 0.006 | N/A |
| | | | | | CaCl2, 21 mM MgCl2 | | | |
| S61 | TST | Colon | chop | Tris | 146 mM NaCl, 1 mM | Tween | 0.03 | N/A |
| | | | | | CaCl2, 21 mM MgCl2 | | | |
| S62 | TST | Colon | chop | Tris | 146 mM NaCl, 1 mM | Tween | 0.03 | N/A |
| | | | | | CaCl2, 21 mM MgCl2 | | | |
| S63 | Sigma-Aldrich | Colon | chop | EZ | N/A | EZ | N/A | N/A |
| | EZ prep | | | | | | | |
| S64 | Sigma-Aldrich | Colon | chop | EZ | N/A | EZ | N/A | N/A |
| | EZ prep | | | | | | | |
| S65 | Sigma-Aldrich | Colon | chop | EZ | N/A | EZ | N/A | N/A |
| | EZ prep | | | | | | | |
| S66 | NST | Colon | grind | Tris | 146 mM NaCl, 1 mM | NP40 | 0.2 | N/A |
| | | | | | CaCl2, 21 mM MgCl2 | | | |
| S67 | NST | Colon | grind | Tris | 146 mM NaCl, 1 mM | NP40 | 0.2 | N/A |
| | | | | | CaCl2, 21 mM MgCl2 | | | |
| S68 | NSTn | Colon | chop | Tricine | 146 mM NaCl, 1 mM | NP40 | 0.2 | N/A |
| | | | | | CaCl2, 21 mM MgCl2 | | | |
| S69 | NST | Colon | grind | Tris | 146 mM NaCl, 1 mM | NP40 | 0.2 | N/A |
| | | | | | CaCl2, 21 mM MgCl2 | | | |
| S70 | NSTn | Colon | chop | Tricine | 146 mM NaCl, 1 mM | NP40 | 0.2 | Polyamines |
| | | | | | CaCl2, 21 mM MgCl2 | | | |
| S71 | NSTn | Colon | chop | Tricine | 146 mM NaCl, 1 mM | NP40 | 0.2 | N/A |
| | | | | | CaCl2, 21 mM MgCl2 | | | |
| S72 | NSTn | Colon | chop | Tricine | 146 mM NaCl, 1 mM | NP40 | 0.2 | N/A |
| | | | | | CaCl2, 21 mM MgCl2 | | | |
| S73 | NSTn | Colon | chop | Tricine | 146 mM NaCl, 1 mM | NP40 | 0.2 | Polyamines |
| | | | | | CaCl2, 21 mM MgCl2 | | | |
| S74 | NST | Colon | chop | Tris | 146 mM NaCl, 1 mM | NP40 | 0.2 | Polyamines |
| | | | | | CaCl2, 21 mM MgCl2 | | | |
| S75 | Sigma-Aldrich | Colon | grind | EZ | N/A | EZ | N/A | N/A |
| | EZ prep | | | | | | | |
| S76 | Sigma-Aldrich | Colon | grind | EZ | N/A | EZ | N/A | N/A |
| | EZ prep | | | | | | | |
| S77 | Sigma-Aldrich | Colon | grind | EZ | N/A | EZ | N/A | N/A |
| | EZ prep | | | | | | | |
| S78 | Sigma-Aldrich | Colon | grind | EZ | N/A | EZ | N/A | N/A |
| | EZ prep | | | | | | | |
| S79 | Sigma-Aldrich | Brain | dounce | EZ | N/A | EZ | N/A | N/A |
| | EZ prep | | | | | | | |
| S80 | NST | Brain | chop | Tris | 146 mM NaCl, 1 mM | NP40 | 0.2 | N/A |
| | | | | | CaCl2, 21 mM MgCl2 | | | |
| S81 | Sigma-Aldrich | Colon | grind | EZ | N/A | EZ | N/A | N/A |
| | EZ prep | | | | | | | |
| S82 | NST | Colon | chop | Tris | 146 mM NaCl, 1 mM | NP40 | 0.2 | N/A |
| | | | | | CaCl2, 21 mM MgCl2 | | | |
| S83 | Sigma-Aldrich | Colon | grind | EZ | N/A | EZ | N/A | N/A |
| | EZ prep | | | | | | | |
| S84 | NST | Colon | chop | Tris | 146 mM NaCl, 1 mM | NP40 | 0.2 | N/A |
| | | | | | CaCl2, 21 mM MgCl2 | | | |
| S85 | Sigma-Aldrich | Brain | dounce | EZ | N/A | EZ | N/A | N/A |
| | EZ prep | | | | | | | |
| S86 | NST | Brain | chop | Tris | 146 mM NaCl, 1 mM | NP40 | 0.2 | N/A |
| | | | | | CaCl2, 21 mM MgCl2 | | | |
| S87 | NST | Colon | chop | Tris | 146 mM NaCl, 1 mM | NP40 | 0.2 | N/A |
| | | | | | CaCl2, 21 mM MgCl2 | | | |
| S88 | NSTn | Colon | chop | Tricine | 146 mM NaCl, 1 mM | NP40 | 0.2 | Polyamines |
| | | | | | CaCl2, 21 mM MgCl2 | | | |
| S89 | TST | Colon | chop | Tris | 146 mM NaCl, 1 mM | Tween | 0.03 | N/A |
| | | | | | CaCl2, 21 mM MgCl2 | | | |
| S90 | CST | Colon | chop | Tris | 146 mM NaCl, 1 mM | CHAPS | 0.49 | N/A |
| | | | | | CaCl2, 21 mM MgCl2 | | | |
| S91 | NST | Colon | chop | Tris | 146 mM NaCl, 1 mM | NP40 | 0.2 | N/A |
| | | | | | CaCl2, 21 mM MgCl2 | | | |
| S92 | NSTn | Colon | chop | Tricine | 146 mM NaCl, 1 mM | NP40 | 0.2 | Polyamines |
| | | | | | CaCl2, 21 mM MgCl2 | | | |
| S93 | TST | Colon | chop | Tris | 146 mM NaCl, 1 mM | Tween | 0.03 | N/A |
| | | | | | CaCl2, 21 mM MgCl2 | | | |
| S94 | CST | Colon | chop | Tris | 146 mM NaCl, 1 mM | CHAPS | 0.49 | N/A |
| | | | | | CaCl2, 21 mM MgCl2 | | | |
| S95 | TST | Colon | chop | Tris | 146 mM NaCl, 1 mM | Tween | 0.03 | N/A |
| | | | | | CaCl2, 21 mM MgCl2 | | | |
| S96 | NST | Colon | chop | Tris | 146 mM NaCl, 1 mM | NP40 | 0.2 | N/A |
| | | | | | CaCl2, 21 mM MgCl2 | | | |
| S97 | NSTn | Colon | chop | Tricine | 146 mM NaCl, 1 mM | NP40 | 0.2 | Polyamines |
| | | | | | CaCl2, 21 mM MgCl2 | | | |
| S98 | CST | Colon | chop | Tris | 146 mM NaCl, 1 mM | CHAPS | 0.49 | N/A |
| | | | | | CaCl2, 21 mM MgCl2 | | | |
| S99 | NST | Brain | dounce | Tris | 5 mM CaCl, 3 mM | NP40 | 0.1 | Sucrose, EDTA, PMSF, |
| | | | | | Mg(Ac)2 | | | β-mercaptoethanol |
| S100 | NST | Brain | dounce | Tris | 5 mM CaCl, 3 mM | NP40 | 0.1 | Sucrose, EDTA, PMSF, |
| | | | | | Mg(Ac)2 | | | β-mercaptoethanol |
| S101 | NST | Colon | dounce | Tris | 5 mM CaCl, 3 mM | NP40 | 0.1 | Sucrose, EDTA, PMSF, |
| | | | | | Mg(Ac)2 | | | β-mercaptoethanol |
| S102 | NST | Colon | dounce | Tris | 5 mM CaCl, 3 mM | NP40 | 0.1 | Sucrose, EDTA, PMSF, |
| | | | | | Mg(Ac)2 | | | β-mercaptoethanol |
| S103 | Sigma-Aldrich | Colon | dounce | EZ | N/A | EZ | N/A | N/A |
| | EZ prep | | | | | | | |
| S104 | Sigma-Aldrich | Colon | dounce | EZ | N/A | EZ | N/A | N/A |
| | EZ prep | | | | | | | |

| | | % | % | % | % | % | % | Number of |
|---|---|---|---|---|---|---|---|---|
| Sample | GFP+ | Exon | Intron | Intergenic | Exon | Intron | Intergenic | detected |
| ID | Sorted | (mean) | (mean) | (mean) | (SD) | (SD) | (SD) | genes (mean) |
| S1 | Yes | 22.21 | 49.22 | 28.55 | 3.01 | 3.38 | 2.65 | 2645.625 |
| S2 | Yes | 28.59 | 52.87 | 18.52 | 3.36 | 3.65 | 0.89 | 2763.28125 |
| S3 | Yes | 14.4 | 13.7 | 71.87 | 1.72 | 0.61 | 2.02 | 1119.78125 |
| S4 | Yes | 11.68 | 18.95 | 69.35 | 2.03 | 1 | 1.77 | 877.5 |
| S5 | Yes | 13.91 | 20.32 | 65.75 | 1.63 | 0.83 | 2.22 | 1512.451613 |
| S6 | Yes | 29.8 | 44.55 | 25.63 | 1.44 | 1.9 | 2.25 | 2281.129032 |
| S7 | Yes | 28.4 | 43.09 | 28.49 | 1.94 | 2.51 | 1.72 | 3326.1875 |
| S8 | Yes | 18.7 | 43.09 | 38.18 | 1.33 | 1.8 | 1.93 | 2918 |
| S9 | Yes | 24.04 | 30.97 | 44.97 | 2.38 | 2.23 | 3.52 | 1881.3125 |
| S10 | Yes | 34.68 | 43.3 | 21.99 | 3.15 | 3.24 | 2.24 | 2438.645161 |
| S11 | Yes | 30.14 | 44.75 | 25.09 | 2.46 | 2.94 | 2.25 | 2548.65625 |
| S12 | Yes | 31.78 | 51.95 | 16.25 | 1.79 | 1.93 | 0.93 | 3005.4375 |
| S13 | Yes | 37.21 | 35.71 | 27.06 | 2.07 | 2.63 | 1.9 | 1715 |
| S14 | Yes | 27.31 | 54.1 | 18.57 | 2.41 | 3.31 | 1.47 | 2360 |
| S15 | Yes | 30.67 | 38.43 | 30.89 | 2.29 | 2.97 | 1.8 | 2432.65625 |
| S16 | Yes | 37 | 35.93 | 27.05 | 2.39 | 3.15 | 1.91 | 2405.1875 |
| S17 | Yes | 36.05 | 38.79 | 25.15 | 3.01 | 3.82 | 1.83 | 2634.65625 |
| S18 | Yes | 56.9 | 21.83 | 21.26 | 2.29 | 2.44 | 0.5 | 4703.53125 |
| S19 | Yes | 56.18 | 26.55 | 17.26 | 2.64 | 2.94 | 0.42 | 4688.258065 |
| S20 | Yes | 53.66 | 27.09 | 19.24 | 2.34 | 2.71 | 0.59 | 4571.53125 |
| S21 | Yes | 23.38 | 21.85 | 54.76 | 2.78 | 1.37 | 2.29 | 1655.65625 |
| S22 | Yes | 22.85 | 38.12 | 39.01 | 2.18 | 2.56 | 3.17 | 2107.53125 |
| S23 | Yes | 17.38 | 15.09 | 67.51 | 3.07 | 1.05 | 3.1 | 1094.21875 |
| S24 | Yes | 31 | 44.22 | 24.77 | 2.41 | 3.79 | 1.63 | 2403.75 |
| S25 | Yes | 33.56 | 43.82 | 22.6 | 2.49 | 3 | 1.66 | 2443.6875 |
| S26 | Yes | 33.47 | 36.61 | 29.91 | 2.91 | 3.31 | 2.4 | 1783.15625 |
| S27 | No | 37.08 | 50.3 | 12.57 | 1.75 | 1.91 | 0.98 | 2463.375 |
| S28 | No | 24 | 61.41 | 14.57 | 2.31 | 2.81 | 1.03 | 3008.09375 |
| S29 | No | 28.29 | 55.55 | 16.13 | 1.95 | 2.41 | 2.08 | 3741.5 |
| S30 | No | 32.53 | 43.75 | 23.7 | 1.93 | 3.17 | 2.51 | 2319.375 |
| S31 | No | 33.03 | 40.8 | 26.16 | 2.81 | 3.71 | 3.11 | 1966.322581 |
| S32 | No | 15.36 | 24.35 | 60.26 | 2.39 | 2.02 | 3.05 | 1691.90625 |
| S33 | Yes | 32.53 | 44.73 | 22.72 | 1.96 | 3.06 | 2.08 | 2331.625 |
| S34 | Yes | 27.05 | 50.42 | 22.51 | 1.85 | 2.87 | 2.57 | 2436.375 |
| S35 | Yes | 27.96 | 52.74 | 19.29 | 0.95 | 2.61 | 2.33 | 2893.5 |
| S36 | Yes | 37.66 | 24.95 | 37.38 | 2.14 | 1.72 | 2.35 | 1525.96875 |
| S37 | Yes | 33.84 | 49.78 | 16.36 | 1.96 | 2.62 | 1.66 | 2783.90625 |
| S38 | Yes | 33.6 | 49.77 | 16.62 | 1.68 | 2.31 | 1.5 | 2768.75 |
| S39 | Yes | 28.47 | 50.31 | 21.21 | 2.51 | 4.02 | 3.19 | 1882.708333 |
| S40 | Yes | 35.76 | 45.91 | 18.31 | 3.36 | 4.29 | 1.59 | 2179.586207 |
| S41 | Yes | 31.76 | 45.95 | 22.27 | 3.24 | 3.84 | 1.91 | 2013.8 |
| S42 | Yes | 32.38 | 46.81 | 20.79 | 3.19 | 4.09 | 1.54 | 1849.827586 |
| S43 | Yes | 26.11 | 54.55 | 19.32 | 2.46 | 4.05 | 2.42 | 2290.37037 |
| S44 | Yes | 33.48 | 44.98 | 21.52 | 2.96 | 3.51 | 2.34 | 2021.769231 |
| S45 | Yes | 36.86 | 37.98 | 25.14 | 2.22 | 3.1 | 2.33 | 2113.5 |
| S46 | Yes | 36.72 | 39.24 | 24.03 | 2.02 | 2.83 | 2.24 | 2379.645161 |
| S47 | Yes | 40.22 | 42.88 | 16.89 | 2.67 | 3.01 | 1.86 | 2690.36 |
| S48 | Yes | 36.99 | 38.49 | 24.51 | 2.05 | 3.05 | 2.72 | 2323.333333 |
| S49 | Yes | 34.21 | 38.8 | 26.98 | 2.26 | 3.22 | 3.25 | 2070.714286 |
| S50 | Yes | 34.25 | 39.19 | 26.55 | 2.28 | 3.32 | 2.09 | 1591.28 |
| S51 | Yes | 26.79 | 28.05 | 45.13 | 2.52 | 1.49 | 2.76 | 1469.34375 |
| S52 | Yes | 26.02 | 27.92 | 46.03 | 2.53 | 1.28 | 2.62 | 1271.59375 |
| S53 | Yes | 35.53 | 48.2 | 16.25 | 1.77 | 2.5 | 1 | 3237.8125 |
| S54 | Yes | 27.88 | 56.27 | 15.83 | 1.93 | 2.35 | 0.83 | 2907.4375 |
| S55 | Yes | 30.97 | 50.99 | 18.02 | 1.76 | 2.71 | 2.27 | 2904.064516 |
| S56 | Yes | 35.23 | 45.12 | 19.63 | 2.88 | 2.92 | 2.16 | 2066.9375 |
| S57 | Yes | 35.38 | 45.25 | 19.34 | 3.11 | 3.64 | 1.83 | 2200.53125 |
| S58 | Yes | 39.43 | 46.38 | 14.18 | 2.9 | 3.39 | 0.66 | 2724.40625 |
| S59 | Yes | 40.92 | 42.7 | 16.37 | 3.05 | 3.23 | 1.3 | 2020.3125 |
| S60 | Yes | 38.95 | 44.28 | 16.76 | 2.55 | 3.22 | 1.63 | 2701.21875 |
| S61 | Yes | 33.71 | 48.2 | 18.06 | 2.76 | 3.66 | 2.28 | 2786.875 |
| S62 | Yes | 39.35 | 42.48 | 18.15 | 2.55 | 2.69 | 1.36 | 2172.125 |
| S63 | Yes | 20.77 | 42.13 | 37.08 | 1.22 | 2.22 | 3.19 | 3024.125 |
| S64 | Yes | 21.71 | 46.17 | 32.1 | 2.08 | 3.12 | 3.56 | 2890.125 |
| S65 | Yes | 24.58 | 43.76 | 31.64 | 2.09 | 1.7 | 2.87 | 3991.6875 |
| S66 | Yes | 25.95 | 55.12 | 18.91 | 1.44 | 2.47 | 1.69 | 3034.28125 |
| S67 | Yes | 28.61 | 56.55 | 14.82 | 1.88 | 2.29 | 0.85 | 3221 |
| S68 | Yes | 24.25 | 56.78 | 18.96 | 1.59 | 2.1 | 1.27 | 2897.875 |
| S69 | Yes | 29.39 | 53.74 | 16.85 | 2.4 | 3.04 | 1.46 | 3265.78125 |
| S70 | Yes | 25.75 | 59.52 | 14.72 | 1.8 | 2.25 | 0.8 | 3713.875 |
| S71 | Yes | 23.87 | 56.7 | 19.41 | 1.31 | 1.95 | 1.67 | 2693.71875 |
| S72 | Yes | 26.62 | 54.25 | 19.11 | 1.76 | 2.62 | 1.49 | 3157.65625 |
| S73 | Yes | 26.56 | 54.31 | 19.11 | 1.96 | 3.16 | 2.19 | 3158.59375 |
| S74 | Yes | 25.51 | 53.14 | 21.32 | 2.36 | 3.01 | 2.3 | 3271.75 |
| S75 | Yes | 15.99 | 24.84 | 59.08 | 1.7 | 0.85 | 1.52 | 1451.364583 |
| S76 | Yes | 20.53 | 26.28 | 53.15 | 2.27 | 1 | 1.66 | 1340.3125 |
| S77 | Yes | 21.29 | 29.59 | 49.07 | 1.44 | 0.71 | 1.34 | 982.46875 |
| S78 | Yes | 21.85 | 28.46 | 49.62 | 1.66 | 0.73 | 1.46 | 857.59375 |
| S79 | Yes | 23.46 | 38.71 | 37.82 | 1.04 | 1.3 | 1.5 | 1852.145833 |
| S80 | Yes | 23.41 | 54.33 | 22.24 | 1.33 | 1.43 | 1.01 | 2627.877778 |
| S81 | Yes | 20.39 | 40.46 | 39.13 | 1.21 | 1.32 | 1.59 | 2217.364583 |
| S82 | Yes | 21.72 | 51.87 | 26.34 | 0.85 | 1.22 | 1.57 | 2745.864583 |
| S83 | Yes | 13.31 | 18.52 | 68.15 | 1.54 | 0.94 | 1.5 | 938.7083333 |
| S84 | Yes | 24.02 | 33.7 | 42.25 | 2.02 | 1.9 | 1.77 | 2147.565217 |
| S85 | Yes | 26.67 | 58.28 | 15.03 | 1.2 | 1.2 | 0.54 | 3367.510417 |
| S86 | Yes | 20.74 | 60.01 | 19.23 | 1.3 | 1.65 | 1.08 | 3558.75 |
| S87 | Yes | 24.64 | 51.5 | 23.84 | 1.62 | 1.79 | 1.23 | 2301.6875 |
| S88 | Yes | 25.9 | 53.52 | 20.55 | 1.61 | 1.97 | 1.23 | 2202.905263 |
| S89 | Yes | 33.61 | 50.64 | 15.74 | 1.05 | 1.24 | 0.64 | 2864.333333 |
| S90 | Yes | 29.75 | 47.92 | 22.3 | 1.53 | 1.91 | 1.42 | 2624.666667 |
| S91 | Yes | 29.31 | 44.36 | 26.3 | 1.68 | 2.07 | 1.23 | 1912.197917 |
| S92 | Yes | 26.43 | 43.98 | 29.56 | 1.23 | 1.84 | 1.55 | 2099.178947 |
| S93 | Yes | 30.68 | 47.06 | 22.24 | 1.24 | 1.6 | 1.06 | 2216.9375 |
| S94 | Yes | 31 | 43.77 | 25.21 | 1.37 | 1.83 | 1.08 | 2259.3125 |
| S95 | Yes | 44.07 | 40.17 | 15.74 | 2.04 | 2.11 | 0.69 | 2087.989583 |
| S96 | Yes | 34.83 | 41.73 | 23.43 | 2.11 | 2.74 | 0.84 | 1829.315789 |
| S97 | Yes | 35.81 | 43.38 | 20.79 | 2.22 | 2.65 | 1.08 | 2062.78125 |
| S98 | Yes | 33.3 | 43.76 | 22.92 | 1.79 | 2.43 | 1.11 | 2302.852632 |
| S99 | Yes | 26.06 | 52.92 | 20.99 | 1.54 | 1.63 | 1.03 | 2472.6875 |
| S100 | Yes | 26.06 | 52.92 | 20.99 | 1.54 | 1.63 | 1.03 | 2472.6875 |
| S101 | Yes | 7.16 | 17.72 | 75.07 | 0.83 | 1.04 | 1.62 | 1393.115789 |
| S102 | Yes | 7.16 | 17.72 | 75.07 | 0.83 | 1.04 | 1.62 | 1393.115789 |
| S103 | Yes | 6.27 | 19.57 | 74.15 | 0.65 | 0.35 | 0.64 | 739.6282723 |
| S104 | Yes | N/A | N/A | N/A | N/A | N/A | N/A | N/A |

| | Number of | ENS | ENS | | | | | |
|---|---|---|---|---|---|---|---|---|
| Sample | detected | score | score | % | % | % | % | |
| ID | genes (SD) | (mean) | (SD) | Contamination | Glia | Neuron | Oligodendrocyte | Other notes |
| S1 | 246.0692998 | 0.517487812 | 0.046710372 | 31.25 | 43.75 | 25 | 0 | |
| S2 | 239.0918817 | 0.571013987 | 0.055967354 | 25 | 53.12 | 21.88 | 0 | |
| S3 | 99.09030516 | 0.140582076 | 0.017994945 | 100 | 0 | 0 | 0 | |
| S4 | 72.01593126 | 0.14157225 | 0.015252253 | 96.88 | 0 | 3.12 | 0 | |
| S5 | 100.0461489 | 0.254010663 | 0.027300561 | 87.1 | 0 | 12.9 | 0 | |
| S6 | 175.0803118 | 0.367383292 | 0.041975178 | 48.39 | 16.13 | 35.48 | 0 | |
| S7 | 234.8703719 | 0.594395928 | 0.049252277 | 18.75 | 46.88 | 34.38 | 0 | |
| S8 | 161.2109726 | 0.510735547 | 0.033370415 | 35.94 | 26.56 | 37.5 | 0 | |
| S9 | 163.0430346 | 0.321009425 | 0.037783865 | 56.25 | 15.62 | 28.12 | 0 | |
| S10 | 310.2750481 | 0.528793814 | 0.0818039 | 45.16 | 45.16 | 9.68 | 0 | |
| S11 | 203.3450042 | 0.53162348 | 0.049800207 | 15.62 | 53.12 | 31.25 | 0 | |
| S12 | 283.1151059 | 0.73283716 | 0.069021494 | 15.62 | 59.38 | 25 | 0 | |
| S13 | 218.3253805 | 0.300466538 | 0.048395745 | 40.62 | 31.25 | 28.12 | 0 | |
| S14 | 214.1698621 | 0.490194685 | 0.049812532 | 37.5 | 43.75 | 18.75 | 0 | |
| S15 | 168.4015016 | 0.372111999 | 0.036967513 | 71.88 | 21.88 | 6.25 | 0 | |
| S16 | 168.4543374 | 0.461930365 | 0.056740355 | 53.12 | 46.88 | 0 | 0 | |
| S17 | 225.9403406 | 0.380320973 | 0.04857395 | 59.38 | 25 | 15.62 | 0 | |
| S18 | 265.949482 | 0.38830923 | 0.03919231 | 96.88 | 0 | 3.12 | 0 | |
| S19 | 286.9543554 | 0.558661729 | 0.056283988 | 83.87 | 12.9 | 3.23 | 0 | |
| S20 | 322.5542308 | 0.466359257 | 0.04562064 | 84.38 | 9.38 | 6.25 | 0 | |
| S21 | 135.1935635 | 0.292462761 | 0.030285883 | 50 | 28.12 | 21.88 | 0 | |
| S22 | 143.7087387 | 0.547637604 | 0.04331021 | 25 | 68.75 | 6.25 | 0 | |
| S23 | 99.48560982 | 0.165875827 | 0.023523719 | 100 | 0 | 0 | 0 | |
| S24 | 269.939345 | 0.479135709 | 0.0507139 | 18.75 | 43.75 | 37.5 | 0 | |
| S25 | 169.5745711 | 0.605212146 | 0.061010943 | 18.75 | 68.75 | 12.5 | 0 | |
| S26 | 176.4810377 | 0.37780399 | 0.048632415 | 34.38 | 53.12 | 12.5 | 0 | |
| S27 | 103.630574 | 0.63298758 | 0.034426981 | 3.12 | 0 | 96.88 | 0 | |
| S28 | 240.7179925 | 0.401384304 | 0.040879496 | 28.12 | 3.12 | 68.75 | 0 | |
| S29 | 366.155198 | 0.586737757 | 0.058422585 | 25 | 3.12 | 71.88 | 0 | |
| S30 | 153.9019951 | 0.090131698 | 0.016136935 | 87.5 | 12.5 | 0 | 0 | |
| S31 | 195.3795335 | 0.070591285 | 0.019138342 | 90.32 | 6.45 | 3.23 | 0 | |
| S32 | 144.9514961 | 0.094854465 | 0.013770037 | 100 | 0 | 0 | 0 | |
| S33 | 184.4068453 | 0.524922893 | 0.058846544 | 31.25 | 56.25 | 12.5 | 0 | |
| S34 | 173.595474 | 0.65842355 | 0.05161194 | 15.62 | 68.75 | 15.62 | 0 | |
| S35 | 186.6460446 | 0.739473309 | 0.067441152 | 15.62 | 71.88 | 12.5 | 0 | |
| S36 | 119.1792453 | 0.127670371 | 0.032200529 | 78.12 | 18.75 | 3.12 | 0 | |
| S37 | 169.3346703 | 0.353067338 | 0.069624828 | 53.12 | 40.62 | 6.25 | 0 | |
| S38 | 160.2833492 | 0.245947409 | 0.059468452 | 68.75 | 28.12 | 3.12 | 0 | |
| S39 | 242.5403782 | 0.401102789 | 0.061582641 | 37.5 | 54.17 | 8.33 | 0 | |
| S40 | 250.4656575 | 0.612086704 | 0.078135069 | 27.59 | 58.62 | 13.79 | 0 | |
| S41 | 185.5913841 | 0.558478448 | 0.05829107 | 20 | 70 | 10 | 0 | |
| S42 | 224.9159507 | 0.36859913 | 0.053696715 | 48.28 | 34.48 | 17.24 | 0 | |
| S43 | 235.4811111 | 0.514082318 | 0.061993498 | 25.93 | 51.85 | 22.22 | 0 | |
| S44 | 176.5703025 | 0.390002047 | 0.06842437 | 34.62 | 65.38 | 0 | 0 | |
| S45 | 142.857189 | 0.408512721 | 0.058249402 | 40 | 53.33 | 6.67 | 0 | |
| S46 | 209.8923869 | 0.113515515 | 0.016334735 | 93.55 | 6.45 | 0 | 0 | |
| S47 | 220.59499 | 0.122575989 | 0.043663634 | 88 | 12 | 0 | 0 | |
| S48 | 180.160707 | 0.061303295 | 0.010546457 | 100 | 0 | 0 | 0 | |
| S49 | 189.2795367 | 0.073396284 | 0.015456326 | 96.43 | 3.57 | 0 | 0 | |
| S50 | 167.8122514 | 0.375565058 | 0.03970777 | 20 | 72 | 8 | 0 | |
| S51 | 173.8214439 | 0.195239054 | 0.039474432 | 75 | 9.38 | 15.62 | 0 | |
| S52 | 112.6758022 | 0.129755651 | 0.025574235 | 93.75 | 3.12 | 3.12 | 0 | |
| S53 | 223.3577452 | 0.685834686 | 0.074344992 | 28.12 | 50 | 21.88 | 0 | |
| S54 | 171.5970168 | 0.731453698 | 0.0600894 | 15.62 | 71.88 | 12.5 | 0 | |
| S55 | 190.1828149 | 0.418916111 | 0.066578726 | 45.16 | 32.26 | 22.58 | 0 | |
| S56 | 211.2054588 | 0.64183187 | 0.076435172 | 25 | 65.62 | 9.38 | 0 | |
| S57 | 220.3067966 | 0.594686956 | 0.072331089 | 25 | 59.38 | 15.62 | 0 | |
| S58 | 254.9191794 | 0.595443051 | 0.066785796 | 28.12 | 43.75 | 28.12 | 0 | |
| S59 | 212.5849092 | 0.639461107 | 0.080444011 | 25 | 71.88 | 3.12 | 0 | |
| S60 | 245.2738953 | 0.505838108 | 0.085442789 | 50 | 40.62 | 9.38 | 0 | |
| S61 | 254.521217 | 0.617510985 | 0.072519785 | 25 | 43.75 | 31.25 | 0 | |
| S62 | 207.7758405 | 0.571861747 | 0.065471268 | 15.62 | 75 | 9.38 | 0 | |
| S63 | 160.770635 | 0.234494003 | 0.037413783 | 81.25 | 6.25 | 12.5 | 0 | |
| S64 | 253.2868081 | 0.559062958 | 0.050066241 | 28.12 | 25 | 46.88 | 0 | |
| S65 | 272.0860182 | 0.638246144 | 0.061210333 | 37.5 | 28.12 | 34.38 | 0 | |
| S66 | 308.0100518 | 0.57944942 | 0.059375935 | 28.12 | 46.88 | 25 | 0 | |
| S67 | 273.4773132 | 0.689994078 | 0.048788258 | 16.13 | 67.74 | 16.13 | 0 | |
| S68 | 226.6177138 | 0.648766914 | 0.051087142 | 9.38 | 62.5 | 28.12 | 0 | |
| S69 | 220.4961539 | 0.651748562 | 0.04536808 | 21.88 | 50 | 28.12 | 0 | |
| S70 | 245.6461351 | 0.59576544 | 0.04513269 | 25 | 53.12 | 21.88 | 0 | |
| S71 | 205.6223256 | 0.672005566 | 0.043062783 | 3.12 | 81.25 | 15.62 | 0 | |
| S72 | 203.7467277 | 0.532447996 | 0.045073891 | 37.5 | 56.25 | 6.25 | 0 | |
| S73 | 180.9742054 | 0.582683534 | 0.046995763 | 15.62 | 59.38 | 25 | 0 | |
| S74 | 204.6925606 | 0.559512898 | 0.049220998 | 31.25 | 46.88 | 21.88 | 0 | |
| S75 | 101.4126743 | 0.121399246 | 0.014331084 | 94.79 | 0 | 5.21 | 0 | |
| S76 | 108.7523798 | 0.119536131 | 0.015173832 | 95.83 | 0 | 4.17 | 0 | |
| S77 | 82.03261273 | 0.113124397 | 0.013307323 | 95.83 | 0 | 4.17 | 0 | |
| S78 | 70.59721054 | 0.09311867 | 0.010695734 | 100 | 0 | 0 | 0 | |
| S79 | 151.8801055 | 0.215888409 | 0.02399188 | 73.96 | 3.12 | 22.92 | 0 | |
| S80 | 140.9983937 | 0.415160488 | 0.023121713 | 14.44 | 5.56 | 60 | 20 | |
| S81 | 160.6604175 | 0.466902297 | 0.030032527 | 48.96 | 31.25 | 17.71 | 2.08 | |
| S82 | 190.3147748 | 0.534450363 | 0.030823653 | 29.17 | 12.5 | 48.96 | 9.38 | |
| S83 | 57.58330334 | 0.083048351 | 0.008050167 | 100 | 0 | 0 | 0 | |
| S84 | 177.1615987 | 0.393485977 | 0.032261487 | 57.61 | 15.22 | 27.17 | 0 | |
| S85 | 184.9098275 | 0.62795891 | 0.026388614 | 9.38 | 0 | 52.08 | 38.54 | |
| S86 | 195.6620408 | 0.462490187 | 0.023863892 | 15.62 | 4.17 | 75 | 5.21 | |
| S87 | 140.1542581 | 0.563629855 | 0.031711401 | 27.08 | 48.96 | 23.96 | 0 | |
| S88 | 121.5645424 | 0.511081234 | 0.027139581 | 24.21 | 55.79 | 20 | 0 | |
| S89 | 102.1500387 | 0.653612274 | 0.044503959 | 31.25 | 50 | 18.75 | 0 | |
| S90 | 133.2338217 | 0.729282333 | 0.030283706 | 11.46 | 60.42 | 28.12 | 0 | |
| S91 | 126.4834576 | 0.416086467 | 0.02910744 | 41.67 | 44.79 | 13.54 | 0 | |
| S92 | 114.4440116 | 0.423227011 | 0.027895465 | 33.68 | 47.37 | 18.95 | 0 | |
| S93 | 96.78193953 | 0.522875462 | 0.032735108 | 27.08 | 57.29 | 15.62 | 0 | |
| S94 | 126.8554428 | 0.543466412 | 0.030369774 | 22.92 | 60.42 | 16.67 | 0 | |
| S95 | 128.5896417 | 0.668102621 | 0.044149615 | 26.04 | 62.5 | 11.46 | 0 | |
| S96 | 134.4131313 | 0.346486871 | 0.0331539 | 57.89 | 25.26 | 16.84 | 0 | |
| S97 | 158.1346585 | 0.366243638 | 0.034520272 | 63.54 | 25 | 11.46 | 0 | |
| S98 | 157.7946225 | 0.434185883 | 0.037375106 | 47.37 | 31.58 | 21.05 | 0 | |
| S99 | 176.2946561 | 0.561490105 | 0.019635153 | 19.79 | 8.33 | 37.5 | 34.38 | Gradient |
| | | | | | | | | purification |
| S100 | 176.2946561 | 0.561490105 | 0.019635153 | 19.79 | 8.33 | 37.5 | 34.38 | Gradient |
| | | | | | | | | purification |
| S101 | 73.3041492 | 0.234474567 | 0.011849926 | 93.68 | 5.26 | 1.05 | 0 | Gradient |
| | | | | | | | | purification |
| S102 | 73.3041492 | 0.234474567 | 0.011849926 | 93.68 | 5.26 | 1.05 | 0 | Gradient |
| | | | | | | | | purification |
| S103 | 17.12959905 | 0.090708806 | 0.005609088 | 99.48 | 0 | 0.52 | 0 | |
| S104 | N/A | N/A | N/A | N/A | N/A | N/A | N/A | |

TABLE 12
Conditions

| | Extraction | | | | | | Detergent | |
|---|---|---|---|---|---|---|---|---|
| Condition | solution | Type | Preparation | Buffer | Salt | Detergent | Concentration | Modifier |
| 1 | NST | Brain | chop | Tris | 146 mM NaCl, 1 mM | NP40 | 0.2 | N/A |
| | | | | | CaCl2, 21 mM MgCl2 | | | |
| 2 | NST | Brain | chop | Tris | 146 mM NaCl, 1 mM | Tween | 0.0012 | N/A |
| | | | | | CaCl2, 21 mM MgCl2 | | | |
| 3 | Sigma-Aldrich | Brain | dounce | EZ | N/A | EZ | N/A | N/A |
| | Nuclei EZ Prep | | | | | | | |
| 4 | NST | Brain | chop | Tris | 146 mM NaCl, 1 mM | NP40 | 0.2 | N/A |
| | | | | | CaCl2, 21 mM MgCl2 | | | |
| 5 | Sigma-Aldrich | Brain | dounce | EZ | N/A | EZ | N/A | N/A |
| | Nuclei EZ Prep | | | | | | | |
| 6 | NST | Brain | dounce | Tris | 5 mM CaCl, 3 mM | NP40 | 0.1 | Sucrose, EDTA, |
| | | | | | Mg(Ac)2 | | | PMSF, - |
| | | | | | | | | mercaptoethanol |
| 7 | NST | Colon | chop | Tris | 146 mM NaCl, 1 mM | NP40 | 0.2 | N/A |
| | | | | | CaCl2, 21 mM MgCl2 | | | |
| 8 | TST | Colon | chop | Tris | 146 mM NaCl, 1 mM | Tween | 0.0012 | N/A |
| | | | | | CaCl2, 21 mM MgCl2 | | | |
| 9 | Sigma-Aldrich | Colon | grind | EZ | N/A | EZ | N/A | N/A |
| | Nuclei EZ Prep | | | | | | | |
| 10 | Sigma-Aldrich | Colon | chop | EZ | N/A | EZ | N/A | N/A |
| | Nuclei EZ Prep | | | | | | | |
| 11 | DSH | Colon | chop | HEPES | 146 mM NaCl, 1 mM | Digitonin | 0.01 | N/A |
| | | | | | CaCl2, 21 mM MgCl2 | | | |
| 12 | NSH | Colon | chop | HEPES | 146 mM NaCl, 1 mM | NP40 | 0.01 | N/A |
| | | | | | CaCl2, 21 mM MgCl2 | | | |
| 13 | NSH | Colon | chop | HEPES | 146 mM NaCl, 1 mM | NP40 | 0.2 | N/A |
| | | | | | CaCl2, 21 mM MgCl2 | | | |
| 14 | TSH | Colon | chop | HEPES | 146 mM NaCl, 1 mM | Tween | 0.0012 | N/A |
| | | | | | CaCl2, 21 mM MgCl2 | | | |
| 15 | TSH | Colon | chop | HEPES | 146 mM NaCl, 1 mM | Tween | 0.006 | N/A |
| | | | | | CaCl2, 21 mM MgCl2 | | | |
| 16 | NSTn | Colon | chop | Tricine | 146 mM NaCl, 1 mM | NP40 | 0.2 | N/A |
| | | | | | CaCl2, 21 mM MgCl2 | | | |
| 17 | NSTn | Colon | chop | Tricine | 146 mM NaCl, 1 mM | NP40 | 0.2 | Polyamines |
| | | | | | CaCl2, 21 mM MgCl2 | | | |
| 18 | CST | Colon | chop | Tris | 146 mM NaCl, 1 mM | CHAPS | 0.0196 | N/A |
| | | | | | CaCl2, 21 mM MgCl2 | | | |
| 19 | CST | Colon | chop | Tris | 146 mM NaCl, 1 mM | CHAPS | 0.098 | N/A |
| | | | | | CaCl2, 21 mM MgCl2 | | | |
| 20 | CST | Colon | chop | Tris | 146 mM NaCl, 1 mM | CHAPS | 0.49 | N/A |
| | | | | | CaCl2, 21 mM MgCl2 | | | |
| 21 | DST | Colon | chop | Tris | 146 mM NaCl, 1 mM | Digitonin | 0.002 | N/A |
| | | | | | CaCl2, 21 mM MgCl2 | | | |
| 22 | DST | Colon | chop | Tris | 146 mM NaCl, 1 mM | Digitonin | 0.01 | N/A |
| | | | | | CaCl2, 21 mM MgCl2 | | | |
| 23 | DST | Colon | chop | Tris | 146 mM NaCl, 1 mM | Digitonin | 0.05 | N/A |
| | | | | | CaCl2, 21 mM MgCl2 | | | |
| 24 | NST | Colon | chop | Tris | 146 mM NaCl, 1 mM | NP40 | 0.001 | N/A |
| | | | | | CaCl2, 21 mM MgCl2 | | | |
| 25 | NST | Colon | chop | Tris | 146 mM NaCl, 1 mM | NP40 | 0.005 | N/A |
| | | | | | CaCl2, 21 mM MgCl2 | | | |
| 26 | NST | Colon | chop | Tris | 146 mM NaCl, 1 mM | NP40 | 0.01 | N/A |
| | | | | | CaCl2, 21 mM MgCl2 | | | |
| 27 | NST | Colon | chop | Tris | 146 mM NaCl, 1 mM | NP40 | 0.025 | N/A |
| | | | | | CaCl2, 21 mM MgCl2 | | | |
| 28 | NST | Colon | chop | Tris | 146 mM NaCl, 1 mM | NP40 | 0.05 | N/A |
| | | | | | CaCl2, 21 mM MgCl2 | | | |
| 29 | NST | Colon | chop | Tris | 146 mM NaCl, 1 mM | NP40 | 0.2 | N/A |
| | | | | | CaCl2, 21 mM MgCl2 | | | |
| 30 | NST | Colon | chop | Tris | 146 mM NaCl, 1 mM | NP40 | 0.2 | Polyamines |
| | | | | | CaCl2, 21 mM MgCl2 | | | |
| 31 | NST | Colon | chop | Tris | 146 mM NaCl, 1 mM | NP40 | 0.2 | Sucrose |
| | | | | | CaCl2, 21 mM MgCl2 | | | |
| 32 | TST | Colon | chop | Tris | 146 mM NaCl, 1 mM | Tween | 0.00024 | N/A |
| | | | | | CaCl2, 21 mM MgCl2 | | | |
| 33 | TST | Colon | chop | Tris | 146 mM NaCl, 1 mM | Tween | 0.0012 | Cytoskeletal drug |
| | | | | | CaCl2, 21 mM MgCl2 | | | |
| 34 | TST | Colon | chop | Tris | 146 mM NaCl, 1 mM | Tween | 0.0012 | N/A |
| | | | | | CaCl2, 21 mM MgCl2 | | | |
| 35 | TST | Colon | chop | Tris | 146 mM NaCl, 1 mM | Tween | 0.0012 | Protease inhibitor |
| | | | | | CaCl2, 21 mM MgCl2 | | | |
| 36 | TST | Colon | chop | Tris | 146 mM NaCl, 1 mM | Tween | 0.0012 | Rnase inhibitor |
| | | | | | CaCl2, 21 mM MgCl2 | | | |
| 37 | TST | Colon | chop | Tris | 146 mM NaCl, 1 mM | Tween | 0.0012 | Translation inhibitor |
| | | | | | CaCl2, 21 mM MgCl2 | | | |
| 38 | TST | Colon | chop | Tris | 146 mM NaCl, 1 mM | Tween | 0.003 | N/A |
| | | | | | CaCl2, 21 mM MgCl2 | | | |
| 39 | TST | Colon | chop | Tris | 146 mM NaCl, 1 mM | Tween | 0.006 | N/A |
| | | | | | CaCl2, 21 mM MgCl2 | | | |
| 40 | TST | Colon | chop | Tris | 146 mM NaCl, 1 mM | Tween | 0.03 | N/A |
| | | | | | CaCl2, 21 mM MgCl2 | | | |
| 41 | Tris only; | Colon | chop | Tris | None | None | 0 | N/A |
| | Hypotonic | | | | | | | |
| 42 | Sigma-Aldrich | Colon | dounce | EZ | N/A | EZ | N/A | N/A |
| | Nuclei EZ Prep | | | | | | | |
| 43 | Sigma-Aldrich | Colon | grind | EZ | N/A | EZ | N/A | N/A |
| | Nuclei EZ Prep | | | | | | | |
| 44 | NST | Colon | grind | Tris | 146 mM NaCl, 1 mM | NP40 | 0.2 | N/A |
| | | | | | CaCl2, 21 mM MgCl2 | | | |
| 45 | NST | Colon | dounce | Tris | 5 mM CaCl, 3 mM | NP40 | 0.1 | Sucrose, EDTA, |
| | | | | | Mg(Ac)2 | | | PMSF, - |
| | | | | | | | | mercaptoethanol |

| | | % | % | % | % | % | % | Number of |
|---|---|---|---|---|---|---|---|---|
| | GFP+ | Exon | Intron | Intergenic | Exon | Intron | Intergenic | detected |
| Condition | Sorted | (mean) | (mean) | (mean) | (SD) | (SD) | (SD) | genes (mean) |
| 1 | No | 24 | 61.41 | 14.57 | 2.31 | 2.81 | 1.03 | 3008.09375 |
| 2 | No | 37.08 | 50.3 | 12.57 | 1.75 | 1.91 | 0.98 | 2463.375 |
| 3 | No | 28.29 | 55.55 | 16.13 | 1.95 | 2.41 | 2.08 | 3741.5 |
| 4 | Yes | 22.02 | 57.28 | 20.68 | 0.93 | 1.12 | 0.75 | 3108.327957 |
| 5 | Yes | 25.05 | 48.44 | 26.49 | 0.8 | 1.13 | 1.14 | 2609.828125 |
| 6 | Yes | 26.06 | 52.92 | 20.99 | 1.08 | 1.15 | 0.72 | 2472.6875 |
| 7 | No | 33.03 | 40.8 | 26.16 | 2.81 | 3.71 | 3.11 | 1966.322581 |
| 8 | No | 32.53 | 43.75 | 23.7 | 1.93 | 3.17 | 2.51 | 2319.375 |
| 9 | No | 15.36 | 24.35 | 60.26 | 2.39 | 2.02 | 3.05 | 1691.90625 |
| 10 | Yes | 22.35 | 44.02 | 33.61 | 1.07 | 1.39 | 1.86 | 3301.979167 |
| 11 | Yes | 35.76 | 45.91 | 18.31 | 3.36 | 4.29 | 1.59 | 2179.586207 |
| 12 | Yes | 34.21 | 38.8 | 26.98 | 2.26 | 3.22 | 3.25 | 2070.714286 |
| 13 | Yes | 30.61 | 48.39 | 20.98 | 2.08 | 2.87 | 1.65 | 1864.716981 |
| 14 | Yes | 32.5 | 42.9 | 24.58 | 1.29 | 1.86 | 1.38 | 2082.195402 |
| 15 | Yes | 40.22 | 42.88 | 16.89 | 2.67 | 3.01 | 1.86 | 2690.36 |
| 16 | Yes | 22.96 | 45.95 | 31.06 | 0.9 | 1.48 | 1.7 | 2590.5875 |
| 17 | Yes | 28.87 | 48.44 | 22.67 | 0.82 | 1.05 | 0.66 | 2355.272966 |
| 18 | Yes | 36.54 | 41.48 | 21.97 | 1.72 | 1.84 | 1.29 | 1956.802083 |
| 19 | Yes | 34.69 | 47.02 | 18.27 | 1.44 | 1.77 | 1.01 | 2688.473684 |
| 20 | Yes | 31.36 | 46.03 | 22.59 | 0.77 | 1.03 | 0.58 | 2422.997389 |
| 21 | Yes | 23.38 | 21.85 | 54.76 | 2.78 | 1.37 | 2.29 | 1655.65625 |
| 22 | Yes | 29.67 | 42.38 | 27.94 | 1.73 | 1.88 | 1.67 | 2187.666667 |
| 23 | Yes | 17.38 | 15.09 | 67.51 | 3.07 | 1.05 | 3.1 | 1094.21875 |
| 24 | Yes | 24.04 | 30.97 | 44.97 | 2.38 | 2.23 | 3.52 | 1881.3125 |
| 25 | Ys | 36.05 | 38.79 | 25.15 | 3.01 | 3.82 | 1.83 | 2634.65625 |
| 26 | Yes | 33.48 | 44.98 | 21.52 | 2.96 | 3.51 | 2.34 | 2021.769231 |
| 27 | Yes | 37 | 35.93 | 27.05 | 2.39 | 3.15 | 1.91 | 2405.1875 |
| 28 | Yes | 28.59 | 52.87 | 18.52 | 3.36 | 3.65 | 0.89 | 2763.28125 |
| 29 | Yes | 26 | 44.93 | 29.04 | 0.66 | 0.79 | 0.6 | 2302.530159 |
| 30 | Yes | 19.8 | 36.99 | 43.19 | 1.61 | 2.61 | 3.24 | 2406.063492 |
| 31 | Yes | 27.31 | 54.1 | 18.57 | 2.41 | 3.31 | 1.47 | 2360 |
| 32 | Yes | 53.66 | 27.09 | 19.24 | 2.34 | 2.71 | 0.59 | 4571.53125 |
| 33 | Yes | 33.84 | 49.78 | 16.36 | 1.96 | 2.62 | 1.66 | 2783.90625 |
| 34 | Yes | 39.19 | 40.74 | 20.06 | 1.37 | 1.58 | 0.85 | 3094.373016 |
| 35 | Yes | 27.96 | 52.74 | 19.29 | 0.95 | 2.61 | 2.33 | 2893.5 |
| 36 | Yes | 33.6 | 49.77 | 16.62 | 1.68 | 2.31 | 1.5 | 2768.75 |
| 37 | Yes | 37.66 | 24.95 | 37.38 | 2.14 | 1.72 | 2.35 | 1525.96875 |
| 38 | Yes | 56.9 | 21.83 | 21.26 | 2.29 | 2.44 | 0.5 | 4703.53125 |
| 39 | Yes | 35.34 | 44.15 | 20.49 | 1.15 | 1.48 | 1.03 | 2718.178862 |
| 40 | Yes | 36.28 | 45 | 18.7 | 0.78 | 0.88 | 0.48 | 2348.481771 |
| 41 | Yes | 14.4 | 13.7 | 71.87 | 1.72 | 0.61 | 2.02 | 1119.78125 |
| 42 | Yes | 6.27 | 19.57 | 74.15 | 0.65 | 0.35 | 0.64 | 739.6282723 |
| 43 | Yes | 19.68 | 28.08 | 52.19 | 0.65 | 0.44 | 0.68 | 1305.21875 |
| 44 | Yes | 27.98 | 55.12 | 16.88 | 1.12 | 1.5 | 0.81 | 3173.189474 |
| 45 | Yes | 7.16 | 17.72 | 75.07 | 0.59 | 0.74 | 1.14 | 1393.115789 |

| | Number of | ENS | ENS | | | | | |
|---|---|---|---|---|---|---|---|---|
| | detected | score | score | % | % | % | % | |
| Condition | genes (SD) | (mean) | (SD) | Contamination | Glia | Neuron | Oligodendrocyte | Other notes |
| 1 | 240.7179925 | 0.401384304 | 0.040879496 | 28.12 | 3.12 | 68.75 | 0 | |
| 2 | 103.630574 | 0.63298758 | 0.034426981 | 3.12 | 0 | 96.88 | 0 | |
| 3 | 366.155198 | 0.586737757 | 0.058422585 | 25 | 3.12 | 71.88 | 0 | |
| 4 | 126.2705059 | 0.439588719 | 0.016685561 | 15.05 | 4.84 | 67.74 | 12.37 | |
| 5 | 131.3222121 | 0.421923659 | 0.023207359 | 41.67 | 1.56 | 37.5 | 19.27 | |
| 6 | 124.3323857 | 0.561490105 | 0.013847756 | 19.79 | 8.33 | 37.5 | 34.38 | Gradient |
| | | | | | | | | purification |
| 7 | 195.3795335 | 0.070591285 | 0.019138342 | 90.32 | 6.45 | 3.23 | 0 | |
| 8 | 153.9019951 | 0.090131698 | 0.016136935 | 87.5 | 12.5 | 0 | 0 | |
| 9 | 144.9514961 | 0.094854465 | 0.013770037 | 100 | 0 | 0 | 0 | |
| 10 | 142.7489853 | 0.477267702 | 0.033965434 | 48.96 | 19.79 | 31.25 | 0 | |
| 11 | 250.4656575 | 0.612086704 | 0.078135069 | 27.59 | 58.62 | 13.79 | 0 | |
| 12 | 189.2795367 | 0.073396284 | 0.015456326 | 96.43 | 3.57 | 0 | 0 | |
| 13 | 163.3765326 | 0.383317768 | 0.040176319 | 43.4 | 43.4 | 13.21 | 0 | |
| 14 | 99.86258915 | 0.490966226 | 0.032647268 | 25.29 | 64.37 | 10.34 | 0 | |
| 15 | 220.59499 | 0.122575989 | 0.043663634 | 88 | 12 | 0 | 0 | |
| 16 | 111.9460862 | 0.517837731 | 0.02437511 | 33.12 | 49.38 | 17.5 | 0 | |
| 17 | 69.25384424 | 0.45411528 | 0.015049033 | 37.8 | 42.78 | 19.42 | 0 | |
| 18 | 115.403659 | 0.553032322 | 0.04193142 | 28.12 | 63.54 | 8.33 | 0 | |
| 19 | 120.6675505 | 0.541130166 | 0.038049419 | 30.53 | 48.42 | 21.05 | 0 | |
| 20 | 69.05119207 | 0.577546689 | 0.017717045 | 25.33 | 52.74 | 21.93 | 0 | |
| 21 | 135.1935635 | 0.292462761 | 0.030285883 | 50 | 28.12 | 21.88 | 0 | |
| 22 | 129.303049 | 0.544853387 | 0.035944082 | 30.11 | 61.29 | 8.6 | 0 | |
| 23 | 99.48560982 | 0.165875827 | 0.023523719 | 100 | 0 | 0 | 0 | |
| 24 | 163.0430346 | 0.321009425 | 0.037783865 | 56.25 | 15.62 | 28.12 | 0 | |
| 25 | 225.9403406 | 0.380320973 | 0.04857395 | 59.38 | 25 | 15.62 | 0 | |
| 26 | 176.5703025 | 0.390002047 | 0.06842437 | 34.62 | 65.38 | 0 | 0 | |
| 27 | 168.4543374 | 0.461930365 | 0.056740355 | 53.12 | 46.88 | 0 | 0 | |
| 28 | 239.0918817 | 0.571013987 | 0.055967354 | 25 | 53.12 | 21.88 | 0 | |
| 29 | 59.37318779 | 0.459541993 | 0.0121788 | 42.06 | 30.48 | 26.03 | 1.43 | |
| 30 | 159.6915756 | 0.409186402 | 0.034190499 | 58.73 | 23.81 | 17.46 | 0 | |
| 31 | 214.1698621 | 0.490194685 | 0.049812532 | 37.5 | 43.75 | 18.75 | 0 | |
| 32 | 322.5542308 | 0.466359257 | 0.04562064 | 84.38 | 9.38 | 6.25 | 0 | |
| 33 | 169.3346703 | 0.353067338 | 0.069624828 | 53.12 | 40.62 | 6.25 | 0 | |
| 34 | 147.8431665 | 0.484808066 | 0.033634941 | 55.56 | 34.13 | 10.32 | 0 | |
| 35 | 186.6460446 | 0.739473309 | 0.067441152 | 15.62 | 71.88 | 12.5 | 0 | |
| 36 | 160.2833492 | 0.245947409 | 0.059468452 | 68.75 | 28.12 | 3.12 | 0 | |
| 37 | 119.1792453 | 0.127670371 | 0.032200529 | 78.12 | 18.75 | 3.12 | 0 | |
| 38 | 265.949482 | 0.38830923 | 0.03919231 | 96.88 | 0 | 3.12 | 0 | |
| 39 | 111.5573251 | 0.461794063 | 0.0377798 | 46.34 | 37.4 | 16.26 | 0 | |
| 40 | 60.56465103 | 0.585300862 | 0.020452456 | 27.86 | 54.95 | 17.19 | 0 | |
| 41 | 99.09030516 | 0.140582076 | 0.017994945 | 100 | 0 | 0 | 0 | |
| 42 | 17.12959905 | 0.090708806 | 0.005609088 | 99.48 | 0 | 0.52 | 0 | |
| 43 | 42.58852379 | 0.165819099 | 0.008309999 | 88.75 | 5.31 | 5.62 | 0.31 | |
| 44 | 154.3900503 | 0.639875283 | 0.029840916 | 22.11 | 54.74 | 23.16 | 0 | |
| 45 | 51.6965525 | 0.234474567 | 0.008356966 | 93.68 | 5.26 | 1.05 | 0 | Gradient |
| | | | | | | | | purification |

Tables 13-17. Summary and marker genes for mouse ENS atlas. (Table 13) Description of each mouse and mouse sample profiled in this study, including model, age, sex, circadian phase, and colon location. Marker genes for mouse (Tables 14 and 15) neurons sequenced with SS2 (Table 14, markers; Table 15, Covariates), (Table 16) mouse glia sequenced with SS2, and (Table 17) all cells from the mouse colon profiled with droplet-based 10× sequencing.

TABLE 13

| Cre_driv- | | | Co- | Time_col- | ~Age |
|---|---|---|---|---|---|
| er | Sample_ID | Gender | lon_order | lected | (weeks) |
| Sox10 | Navin3_S24 | F | All | #N/A | 12 |
| Sox10 | Navin6_S54 | M | All | #N/A | 12 |
| Sox10 | Navin6_S57 | M | All | #N/A | 12 |
| Sox10 | Navin8_S90 | M | All | #N/A | 12 |
| Sox10 | Navin9_S94 | F | All | 2PM | 12 |
| Sox10 | Navin10_S98 | M | All | 7AM | 12 |
| Sox10 | ENS1A_1 | F | 1 | 7AM | 12 |
| Sox10 | ENS1A_2 | F | 2 | 7AM | 12 |
| Sox10 | ENS1A_3 | F | 3 | 7AM | 12 |
| Sox10 | ENS1A_4 | F | 4 | 7AM | 12 |
| Sox10 | ENS1B_1 | F | 1 | 7AM | 12 |
| Sox10 | ENS1B_2 | F | 2 | 7AM | 12 |
| Sox10 | ENS1B_3 | F | 3 | 7AM | 12 |
| Sox10 | ENS1B_4 | F | 4 | 7AM | 12 |
| Sox10 | ENS2_1 | F | 1 | 7PM | 12 |
| Sox10 | ENS2_2 | F | 2 | 7PM | 12 |
| Sox10 | ENS2_3 | F | 3 | 7PM | 12 |
| Sox10 | ENS2_4 | F | 4 | 7PM | 12 |
| Sox10 | ENS3_1 | M | 1 | 7AM | 12 |
| Sox10 | ENS3_2 | M | 2 | 7AM | 12 |
| Sox10 | ENS3_3 | M | 3 | 7AM | 12 |
| Sox10 | ENS3_4 | M | 4 | 7AM | 12 |
| Sox10 | ENS4_1 | M | 1 | 7PM | 12 |
| Sox10 | ENS4_2 | M | 2 | 7PM | 12 |
| Sox10 | ENS4_3 | M | 3 | 7PM | 12 |
| Sox10 | ENS4_4 | M | 4 | 7PM | 12 |
| Sox10 | ENS5_1 | M | 1 | 7AM | 12 |
| Sox10 | ENS5_2 | M | 2 | 7AM | 12 |
| Sox10 | ENS5_3 | M | 3 | 7AM | 12 |
| Sox10 | ENS5_4 | M | 4 | 7AM | 12 |
| Sox10 | ENS6_1 | M | 1 | 7PM | 12 |
| Sox10 | ENS6_2 | M | 2 | 7PM | 12 |
| Sox10 | ENS6_3 | M | 3 | 7PM | 12 |
| Sox10 | ENS6_4 | M | 4 | 7PM | 12 |
| Sox10 | ENS7_1 | M | 1 | 7PM | 12 |
| Sox10 | ENS7_2 | M | 2 | 7PM | 12 |
| Sox10 | ENS7_3 | M | 3 | 7PM | 12 |
| Sox10 | ENS7_4 | M | 4 | 7PM | 12 |
| WNT1 | ENS8_1 | F | 1 | 7AM | 12 |
| WNT1 | ENS8_2 | F | 2 | 7AM | 12 |
| WNT1 | ENS9_1 | M | 1 | 7PM | 12 |
| WNT1 | ENS9_2 | M | 2 | 7PM | 12 |
| WNT1 | ENS9_3 | M | 3 | 7PM | 12 |
| WNT1 | ENS9_4 | M | 4 | 7PM | 12 |
| AGED | ENS10A_1 | F | 1 | 7PM | 52 |
| AGED | ENS10B_1 | F | 1 | 7PM | 52 |
| AGED | ENS10A_2 | F | 2 | 7PM | 52 |
| AGED | ENS10A_3 | F | 3 | 7PM | 52 |
| AGED | ENS10A_4 | F | 4 | 7PM | 52 |
| AGED | ENS10B_4 | F | 4 | 7PM | 52 |
| Uchl1 | ENS11_1 | M | 1 | 7AM | 12 |
| Uchl1 | ENS11_2 | M | 2 | 7AM | 12 |
| Uchl1 | ENS11_3 | M | 4 | 7AM | 12 |
| Uchl1 | ENS11_4 | M | 4 | 7AM | 12 |
| Sox10 | ENS12_1 | M | 1 | 7AM | 12 |
| Sox10 | ENS12_2 | M | 2 | 7AM | 12 |
| Sox10 | ENS12_3 | M | 3 | 7AM | 12 |
| Sox10 | ENS12_4 | M | 4 | 7AM | 12 |
| Uchl1 | ENS14_1 | M | 1 | 7AM | 12 |
| Uchl1 | ENS14_2 | M | 4 | 7AM | 12 |
| Sox10 | ENS13_1 | F | 1 | 7AM | 12 |
| Sox10 | ENS13_2 | F | 4 | 7AM | 12 |
| AGED | ENS15_1 | M | 1 | 7PM | 52 |
| AGED | ENS15_2 | M | 2 | 7PM | 52 |
| AGED | ENS15_3 | M | 3 | 7PM | 52 |
| AGED | ENS15_4 | M | 4 | 7PM | 52 |
| Uchl1 | ENS16A_1 | M | 1 | 7PM | 12 |
| Uchl1 | ENS16A_2 | M | 2 | 7PM | 12 |
| Uchl1 | ENS16A_3 | M | 3 | 7PM | 12 |
| Uchl1 | ENS16A_4 | M | 4 | 7PM | 12 |
| Uchl1 | ENS16B_1 | M | 1 | 7PM | 12 |
| Uchl1 | ENS16B_2 | M | 2 | 7PM | 12 |
| Uchl1 | ENS16B_3 | M | 3 | 7PM | 12 |
| Uchl1 | ENS16B_4 | M | 4 | 7PM | 12 |
| AGED | ENS17_1 | M | 1 | 7AM | 52 |
| AGED | ENS17_2 | M | 2 | 7AM | 52 |
| AGED | ENS17_3 | M | 3 | 7AM | 52 |
| AGED | ENS17_4 | M | 4 | 7AM | 52 |
| Uchl1 | ENS18_1 | F | 1 | 7PM | 12 |
| Uchl1 | ENS18_2 | F | 2 | 7PM | 12 |
| Uchl1 | ENS18_3 | F | 3 | 7PM | 12 |
| Uchl1 | ENS18_4 | F | 4 | 7PM | 12 |
| Uchl1 | ENS19_1 | F | 1 | 7AM | 11 |
| Uchl1 | ENS19_2 | F | 2 | 7AM | 12 |
| Uchl1 | ENS19_3 | F | 2 | 7AM | 11 |
| Uchl1 | ENS19_4 | F | 4 | 7AM | 11 |

TABLE 14

| ident | gene | padjH |
|---|---|---|
| Other_1 | Fam129a | 4.17E−69 |
| Other_1 | Matn1 | 5.45E−48 |
| Other_1 | Atp1a2 | 1.28E−44 |
| Other_1 | Shroom4 | 5.83E−42 |
| Other_1 | Plxnb3 | 3.19E−41 |
| Other_1 | Tacr3 | 1.37E−33 |
| Other_1 | Rasl12 | 1.78E−33 |
| Other_1 | F13b | 3.76E−33 |
| Other_1 | C4b | 7.67E−33 |
| Other_1 | Serpinb9c | 1.67E−32 |
| Other_1 | Wdr69 | 7.07E−31 |
| Other_1 | Bbox1 | 2.46E−30 |
| Other_1 | Tmprss5 | 7.97E−29 |
| Other_1 | 5430428K19Rik | 4.25E−27 |
| Other_1 | Foxp2 | 2.18E−25 |
| Other_1 | Wdr96 | 2.57E−25 |
| Other_1 | Mtrf1 | 7.31E−24 |
| Other_1 | Rad54b | 1.90E−21 |
| Other_1 | Afap1l2 | 3.11E−21 |
| Other_1 | Abca8a | 1.34E−20 |
| Other_1 | Rai14 | 2.19E−20 |
| Other_1 | Kank1 | 4.52E−20 |
| Other_1 | Sox5 | 5.77E−20 |
| Other_1 | Egfbp2 | 6.59E−20 |
| Other_1 | Musk | 1.54E−19 |
| Other_1 | 4930448C13Rik | 5.99E−19 |
| Other_1 | Cdh19 | 1.39E−18 |
| Other_1 | Fzd6 | 1.20E−17 |
| Other_1 | Gm10863 | 1.55E−17 |
| Other_1 | Ccdc114 | 5.32E−16 |
| Other_1 | 2810055G20Rik | 7.07E−16 |
| Other_1 | Dapp1 | 2.69E−15 |
| Other_1 | Lhfp | 3.60E−15 |
| Other_1 | H2-T10 | 3.68E−15 |
| Other_1 | Plac9a | 7.76E−15 |
| Other_1 | Col18a1 | 1.55E−14 |
| Other_1 | Lpar1 | 3.73E−14 |
| Other_1 | Chi3l1 | 3.78E−14 |
| Other_1 | Icos | 3.78E−14 |
| Other_1 | Sox13 | 5.72E−14 |
| Other_1 | Trabd2b | 1.81E−13 |
| Other_1 | Col12a1 | 2.06E−13 |
| Other_1 | Ntng2 | 8.43E−13 |
| Other_1 | Agmo | 1.30E−12 |
| Other_1 | Col11a1 | 5.68E−12 |
| Other_1 | 9130409I23Rik | 2.51E−11 |
| Other_1 | Loxl3 | 1.03E−10 |
| Other_1 | Kif27 | 1.84E−10 |
| Other_1 | 2810025M15Rik | 2.57E−10 |
| Other_1 | Gm10389 | 2.75E−10 |
| Other_1 | Upb1 | 2.78E−10 |
| Other_1 | Cyp39a1 | 1.58E−09 |
| Other_1 | Sox6 | 1.61E−09 |
| Other_1 | Nckap5 | 1.86E−09 |
| Other_1 | C1qtnf7 | 2.30E−09 |
| Other_1 | 2610307P16Rik | 2.51E−09 |
| Other_1 | Sall1 | 2.94E−09 |
| Other_1 | 4930432M17Rik | 3.79E−09 |
| Other_1 | Etl4 | 4.03E−09 |
| Other_1 | Dock5 | 7.00E−09 |
| Other_1 | Smoc1 | 8.22E−09 |
| Other_1 | Zcchc24 | 9.94E−09 |
| Other_1 | Wwtr1 | 1.03E−08 |
| Other_1 | Frzb | 1.04E−08 |
| Other_1 | Il1rap | 1.21E−08 |
| Other_1 | Hyal4 | 1.32E−08 |
| Other_1 | Baz1a | 1.64E−08 |
| Other_1 | Prdm16 | 2.25E−08 |
| Other_1 | Gsn | 2.56E−08 |
| Other_1 | Apoc3 | 4.72E−08 |
| Other_1 | Nod1 | 7.80E−08 |
| Other_1 | Pmepa1 | 1.09E−07 |
| Other_1 | Fam107a | 1.28E−07 |
| Other_1 | Slc7a2 | 1.30E−07 |
| Other_1 | Dydc2 | 1.37E−07 |
| Other_1 | Sox10 | 1.45E−07 |
| Other_1 | Nhp2 | 1.74E−07 |
| Other_1 | Tgfb2 | 1.95E−07 |
| Other_1 | Plac9b | 2.24E−07 |
| Other_1 | Oosp1 | 2.39E−07 |
| Other_1 | Npm3-ps1 | 2.95E−07 |
| Other_1 | Abca15 | 4.96E−07 |
| Other_1 | Apoe | 5.11E−07 |
| Other_1 | Gm3143 | 6.47E−07 |
| Other_1 | Prodh | 7.24E−07 |
| Other_1 | Car12 | 1.00E−06 |
| Other_1 | Cmtm5 | 1.32E−06 |
| Other_1 | Rreb1 | 1.75E−06 |
| Other_1 | 1700112E06Rik | 2.41E−06 |
| Other_1 | Stard8 | 2.59E−06 |
| Other_1 | Ddx49 | 2.63E−06 |
| Other_1 | Acox2 | 2.63E−06 |
| Other_1 | Gli3 | 2.80E−06 |
| Other_1 | Kctd1 | 4.35E−06 |
| Other_1 | Gbp5 | 4.64E−06 |
| Other_1 | 1700010I14Rik | 5.81E−06 |
| Other_1 | Mrvi1 | 5.92E−06 |
| Other_1 | Megf10 | 6.01E−06 |
| Other_1 | AI661453 | 6.01E−06 |
| Other_1 | Mob3b | 7.40E−06 |
| Other_1 | Kirrel | 7.46E−06 |
| Other_1 | Bhmt | 9.22E−06 |
| Other_1 | Ajap1 | 1.13E−05 |
| Other_1 | Olfml1 | 1.85E−05 |
| Other_1 | Ankle1 | 1.85E−05 |
| Other_1 | Cml3 | 2.93E−05 |
| Other_1 | Tmem254a | 3.78E−05 |
| Other_1 | Slc35f2 | 3.95E−05 |
| Other_1 | Bcl2l12 | 4.13E−05 |
| Other_1 | Entpd2 | 5.55E−05 |
| Other_1 | Gcnt1 | 6.44E−05 |
| Other_1 | Sox2ot | 7.47E−05 |
| Other_1 | Ikbke | 8.38E−05 |
| Other_1 | 1700047M11Rik | 8.63E−05 |
| Other_1 | Megf6 | 1.48E−04 |
| Other_1 | Tbx18 | 2.01E−04 |
| Other_1 | Myh11 | 3.46E−04 |
| Other_1 | Myof | 4.10E−04 |
| Other_1 | Gpr17 | 4.55E−04 |
| Other_1 | Ptgfrn | 4.85E−04 |
| Other_1 | Efhd1 | 6.72E−04 |
| Other_1 | Myh6 | 7.64E−04 |
| Other_1 | Fendrr | 9.37E−04 |
| Other_1 | Col6a3 | 9.57E−04 |
| Other_1 | Fhl4 | 1.47E−03 |
| Other_1 | Col9a2 | 1.90E−03 |
| Other_1 | Lcp2 | 2.13E−03 |
| Other_1 | Mapk15 | 2.47E−03 |
| Other_1 | Kcnj10 | 5.38E−03 |
| Other_1 | Car13 | 6.95E−03 |
| Other_1 | Cep72 | 7.24E−03 |
| Other_1 | 4932435O22Rik | 8.43E−03 |
| Other_1 | Tex36 | 1.01E−02 |
| Other_1 | Lims2 | 1.29E−02 |
| Other_1 | Rrad | 1.70E−02 |
| Other_1 | S1pr3 | 1.80E−02 |
| Other_1 | Nfatc4 | 2.45E−02 |
| Other_1 | Evc | 2.61E−02 |
| Other_1 | Arhgef19 | 4.94E−02 |
| Other_2 | Arhgef38 | 6.44E−117 |
| Other_2 | Agr2 | 4.04E−86 |
| Other_2 | Oit1 | 4.43E−83 |
| Other_2 | Cphx1 | 1.01E−81 |
| Other_2 | Shroom3 | 9.76E−69 |
| Other_2 | Sh2d1b2 | 5.63E−58 |
| Other_2 | Mecom | 2.55E−55 |
| Other_2 | Gm14204 | 6.90E−55 |
| Other_2 | Gm10415 | 2.58E−49 |
| Other_2 | Gm7073 | 1.44E−46 |
| Other_2 | Slc12a8 | 4.50E−45 |
| Other_2 | Sprr2b | 4.81E−44 |
| Other_2 | Tnfaip8 | 1.83E−41 |
| Other_2 | Galnt12 | 1.77E−36 |
| Other_2 | Rasef | 3.64E−35 |
| Other_2 | Nipsnap3a | 5.48E−31 |
| Other_2 | Atp8b1 | 6.22E−30 |
| Other_2 | Sytl2 | 9.47E−30 |
| Other_2 | Mctp2 | 9.54E−30 |
| Other_2 | Fam3b | 7.05E−29 |
| Other_2 | Cdcp1 | 7.94E−29 |
| Other_2 | Eps8 | 1.49E−28 |
| Other_2 | Tff3 | 3.22E−28 |
| Other_2 | Muc2 | 2.06E−27 |
| Other_2 | Capn13 | 2.16E−27 |
| Other_2 | 1700120E14Rik | 2.09E−26 |
| Other_2 | Spink4 | 2.16E−26 |
| Other_2 | BC030870 | 1.78E−25 |
| Other_2 | Sprr2a1 | 7.41E−24 |
| Other_2 | D930020B18Rik | 6.05E−23 |
| Other_2 | Tmem236 | 3.33E−22 |
| Other_2 | Ano9 | 2.17E−21 |
| Other_2 | Myo5b | 2.89E−21 |
| Other_2 | Fcamr | 5.37E−21 |
| Other_2 | Itgal | 5.90E−21 |
| Other_2 | Slfn4 | 5.90E−21 |
| Other_2 | Nupr1 | 7.13E−21 |
| Other_2 | Hepacam2 | 1.45E−20 |
| Other_2 | 1810007I06Rik | 2.54E−20 |
| Other_2 | Sprr2a2 | 2.54E−20 |
| Other_2 | Fermt1 | 4.22E−20 |
| Other_2 | E230025N22Rik | 4.93E−20 |
| Other_2 | Mroh4 | 7.84E−20 |
| Other_2 | Gm609 | 1.27E−19 |
| Other_2 | Myo5c | 2.03E−19 |
| Other_2 | Zan | 2.58E−19 |
| Other_2 | Gm10754 | 5.79E−19 |
| Other_2 | Slc15a1 | 6.44E−19 |
| Other_2 | Saa1 | 1.31E−18 |
| Other_2 | Mrgpra9 | 1.56E−18 |
| Other_2 | Blnk | 1.60E−18 |
| Other_2 | Abcg5 | 2.86E−18 |
| Other_2 | Rbm47 | 3.97E−18 |
| Other_2 | Crxos1 | 4.13E−18 |
| Other_2 | Plac8 | 5.43E−18 |
| Other_2 | Ano7 | 1.92E−17 |
| Other_2 | Spink3 | 1.95E−17 |
| Other_2 | Myo3a | 3.13E−17 |
| Other_2 | Frmd7 | 4.11E−17 |
| Other_2 | 4921508A21Rik | 4.33E−17 |
| Other_2 | 4930511M11Rik | 1.13E−16 |
| Other_2 | Spdef | 2.28E−16 |
| Other_2 | Abo | 2.76E−16 |
| Other_2 | Epcam | 7.96E−16 |
| Other_2 | Rdh18-ps | 1.23E−15 |
| Other_2 | Slc34a2 | 1.67E−15 |
| Other_2 | 4930515L19Rik | 3.79E−15 |
| Other_2 | Ms4a8a | 4.79E−15 |
| Other_2 | Lypd8 | 6.50E−15 |
| Other_2 | Atp2c2 | 8.54E−15 |
| Other_2 | Tmem45b | 2.43E−14 |
| Other_2 | Capn8 | 2.79E−14 |
| Other_2 | Slc22a14 | 2.79E−14 |
| Other_2 | Mlph | 6.98E−14 |
| Other_2 | Ano1 | 9.16E−14 |
| Other_2 | Atp2a3 | 9.83E−14 |
| Other_2 | Shroom2 | 1.55E−13 |
| Other_2 | Gpr128 | 2.41E−13 |
| Other_2 | Hgfac | 2.72E−13 |
| Other_2 | Pld1 | 3.68E−13 |
| Other_2 | Ern2 | 4.18E−13 |
| Other_2 | Mob3b | 5.18E−13 |
| Other_2 | Arhgap18 | 1.30E−12 |
| Other_2 | Gm5414 | 1.70E−12 |
| Other_2 | Cdh17 | 1.90E−12 |
| Other_2 | Esrp1 | 1.94E−12 |
| Other_2 | Sh2d4a | 2.78E−12 |
| Other_2 | Cyp2d13 | 2.79E−12 |
| Other_2 | Bsph2 | 2.79E−12 |
| Other_2 | Serpina9 | 3.08E−12 |
| Other_2 | Zg16 | 3.27E−12 |
| Other_2 | Spink5 | 4.62E−12 |
| Other_2 | Rab11fip1 | 4.77E−12 |
| Other_2 | Glis3 | 6.24E−12 |
| Other_2 | Best2 | 1.34E−11 |
| Other_2 | Capn9 | 1.35E−11 |
| Other_2 | Cpm | 1.64E−11 |
| Other_2 | Cmtm8 | 1.64E−11 |
| Other_2 | B3galt5 | 1.64E−11 |
| Other_2 | Muc13 | 2.83E−11 |
| Other_2 | Clec2d | 2.84E−11 |
| Other_2 | Slc17a9 | 3.15E−11 |
| Other_2 | Slc26a9 | 3.69E−11 |
| Other_2 | Cyp2d34 | 5.36E−11 |
| Other_2 | 9030619P08Rik | 1.11E−10 |
| Other_2 | Kit | 1.23E−10 |
| Other_2 | Gm19510 | 1.75E−10 |
| Other_2 | 5830428M24Rik | 1.35E−09 |
| Other_2 | 6030408B16Rik | 1.86E−09 |
| Other_2 | Gata6 | 2.55E−09 |
| Other_2 | Kcnv2 | 6.86E−09 |
| Other_2 | Hoxa11as | 7.96E−09 |
| Other_2 | Cyp4f40 | 6.23E−08 |
| Other_2 | Hpd | 1.06E−07 |
| Other_2 | Abcc2 | 1.65E−07 |
| Other_2 | Vmn1r63 | 2.35E−07 |
| Other_2 | Tmem82 | 3.95E−07 |
| Other_2 | Tmc8 | 1.94E−06 |
| Other_2 | Dsp | 2.06E−06 |
| Other_2 | Noxa1 | 2.21E−06 |
| Other_2 | Trpv3 | 5.56E−06 |
| Other_2 | Entpd8 | 1.32E−05 |
| Other_2 | Krt12 | 1.40E−05 |
| Other_2 | Gm53 | 1.51E−05 |
| Other_2 | Cdh16 | 4.86E−05 |
| Other_2 | Hoxa11 | 1.04E−04 |
| Other_2 | Rasal1 | 1.90E−04 |
| Other_2 | Duox2 | 2.95E−04 |
| Other_2 | Naip6 | 2.99E−04 |
| Other_2 | Kif12 | 3.69E−04 |
| Other_2 | Gnrhr | 4.75E−04 |
| Other_2 | Hopx | 5.58E−04 |
| Other_2 | Ppp1r3b | 6.67E−04 |
| Other_2 | Cyp2d12 | 6.92E−04 |
| Other_2 | Gm14812 | 7.32E−04 |
| Other_2 | Mrgprb1 | 9.48E−04 |
| Other_2 | Pla2g4d | 1.16E−03 |
| Other_2 | Hes2 | 1.59E−03 |
| Other_2 | Cyp2d11 | 2.34E−03 |
| Other_2 | Slc23a3 | 2.61E−03 |
| Other_2 | Ccdc42 | 3.01E−03 |
| Other_2 | Shh | 3.13E−03 |
| Other_2 | Slfn2 | 3.52E−03 |
| Other_2 | Unc5cl | 3.84E−03 |
| Other_2 | Lrrc66 | 4.09E−03 |
| Other_2 | Mir192 | 5.77E−03 |
| Other_2 | Scnn1g | 6.96E−03 |
| Other_2 | P2ry4 | 8.39E−03 |
| Other_2 | Pla2g2c | 1.01E−02 |
| Other_2 | Slc34a1 | 1.03E−02 |
| Other_2 | AF067063 | 1.17E−02 |
| Other_2 | Retnla | 2.05E−02 |
| Other_2 | Rbbp8nl | 2.35E−02 |
| Other_2 | Hbegf | 4.31E−02 |
| Other_2 | Tnfsf15 | 4.35E−02 |
| Other_2 | Gm9926 | 4.96E−02 |
| PEMN_1 | Cntn4 | 2.42E−140 |
| PEMN_1 | Fstl4 | 4.51E−105 |
| PEMN_1 | Car10 | 1.00E−103 |
| PEMN_1 | Zcchc16 | 6.71E−95 |
| PEMN_1 | Cntn5 | 1.95E−90 |
| PEMN_1 | Csmd3 | 3.57E−89 |
| PEMN_1 | Nxph1 | 3.55E−88 |
| PEMN_1 | Cacna2d3 | 1.30E−84 |
| PEMN_1 | Shc4 | 3.16E−82 |
| PEMN_1 | Dock10 | 4.59E−80 |
| PEMN_1 | Lama2 | 1.06E−78 |
| PEMN_1 | Unc5d | 1.17E−68 |
| PEMN_1 | Ntrk2 | 5.06E−66 |
| PEMN_1 | Gda | 1.57E−62 |
| PEMN_1 | Trpc5 | 1.57E−62 |
| PEMN_1 | Thsd4 | 1.69E−59 |
| PEMN_1 | Adamts12 | 9.02E−59 |
| PEMN_1 | Agtr1a | 2.47E−57 |
| PEMN_1 | Lrp1b | 1.30E−55 |
| PEMN_1 | Synpr | 5.82E−49 |
| PEMN_1 | Adgb | 2.32E−45 |
| PEMN_1 | Antxr2 | 4.93E−45 |
| PEMN_1 | Fgfr2 | 1.09E−41 |
| PEMN_1 | Pion | 1.30E−40 |
| PEMN_1 | Tpd52l1 | 1.51E−40 |
| PEMN_1 | Tac1 | 7.75E−39 |
| PEMN_1 | 5530401A14Rik | 9.60E−38 |
| PEMN_1 | Ccdc60 | 6.16E−37 |
| PEMN_1 | Hgf | 8.76E−36 |
| PEMN_1 | Crispld1 | 2.12E−35 |
| PEMN_1 | Prkg1 | 4.67E−35 |
| PEMN_1 | Kctd8 | 3.50E−34 |
| PEMN_1 | Elfn1 | 5.84E−34 |
| PEMN_1 | Stk32a | 1.27E−32 |
| PEMN_1 | Colq | 6.58E−32 |
| PEMN_1 | Spock3 | 1.13E−31 |
| PEMN_1 | 2610316D01Rik | 2.11E−31 |
| PEMN_1 | Erbb4 | 2.21E−31 |
| PEMN_1 | Pcdh10 | 3.54E−31 |
| PEMN_1 | Dlgap2 | 6.54E−31 |
| PEMN_1 | Tmem164 | 1.99E−30 |
| PEMN_1 | Prkcb | 3.25E−30 |
| PEMN_1 | Olfm3 | 6.46E−30 |
| PEMN_1 | Sh3rf3 | 7.06E−30 |
| PEMN_1 | Slit1 | 7.35E−30 |
| PEMN_1 | Syt16 | 4.37E−29 |
| PEMN_1 | Pdlim3 | 5.78E−29 |
| PEMN_1 | Gria1 | 1.56E−28 |
| PEMN_1 | Lsamp | 2.79E−28 |
| PEMN_1 | Oprk1 | 1.22E−27 |
| PEMN_1 | Gpc6 | 1.22E−27 |
| PEMN_1 | Mir669b | 1.57E−27 |
| PEMN_1 | Cacna1e | 2.98E−27 |
| PEMN_1 | Ralyl | 6.24E−27 |
| PEMN_1 | Atp2b2 | 9.58E−27 |
| PEMN_1 | Dmd | 1.21E−26 |
| PEMN_1 | Amigo2 | 2.18E−26 |
| PEMN_1 | Gulo | 4.03E−26 |
| PEMN_1 | Calcrl | 1.13E−25 |
| PEMN_1 | Fam19a5 | 1.22E−25 |
| PEMN_1 | Pgm5 | 3.72E−25 |
| PEMN_1 | Dach1 | 6.94E−25 |
| PEMN_1 | Grik2 | 7.85E−25 |
| PEMN_1 | Grip1 | 8.12E−25 |
| PEMN_1 | Pld5 | 9.31E−25 |
| PEMN_1 | Neto1 | 1.32E−24 |
| PEMN_1 | Nebl | 1.84E−24 |
| PEMN_1 | Kcnc2 | 4.09E−24 |
| PEMN_1 | Ltbp4 | 6.53E−24 |
| PEMN_1 | D330022K07Rik | 6.80E−24 |
| PEMN_1 | Frem1 | 9.78E−24 |
| PEMN_1 | Rxfp3 | 2.67E−23 |
| PEMN_1 | Tenm1 | 2.95E−23 |
| PEMN_1 | Asic2 | 3.44E−23 |
| PEMN_1 | Sorbs2 | 6.21E−23 |
| PEMN_1 | Cntn3 | 7.09E−23 |
| PEMN_1 | Ust | 7.82E−23 |
| PEMN_1 | Efnb2 | 1.74E−22 |
| PEMN_1 | Epb4.1l5 | 1.74E−22 |
| PEMN_1 | Gas7 | 1.96E−22 |
| PEMN_1 | Cdh18 | 2.46E−22 |
| PEMN_1 | Casz1 | 2.65E−22 |
| PEMN_1 | Ogfrl1 | 3.32E−22 |
| PEMN_1 | Cnr1 | 7.66E−22 |
| PEMN_1 | Kcnd2 | 8.34E−22 |
| PEMN_1 | Pmp22 | 1.83E−21 |
| PEMN_1 | Meis1 | 1.97E−21 |
| PEMN_1 | Ets1 | 2.31E−21 |
| PEMN_1 | Ryr3 | 7.01E−21 |
| PEMN_1 | Pde1c | 1.57E−20 |
| PEMN_1 | Slc16a12 | 2.75E−20 |
| PEMN_1 | Reln | 3.17E−20 |
| PEMN_1 | Hs6st1 | 5.23E−20 |
| PEMN_1 | Tox | 5.33E−20 |
| PEMN_1 | Atrnl1 | 7.23E−20 |
| PEMN_1 | Parvb | 1.93E−19 |
| PEMN_1 | Rimbp2 | 3.08E−19 |
| PEMN_1 | Sec14l5 | 4.08E−19 |
| PEMN_1 | Pcsk1 | 5.27E−19 |
| PEMN_1 | Epha6 | 9.23E−19 |
| PEMN_1 | Sertm1 | 1.32E−15 |
| PEMN_1 | Itgax | 5.36E−15 |
| PEMN_1 | F730043M19Rik | 3.79E−14 |
| PEMN_1 | Crhbp | 1.63E−11 |
| PEMN_1 | Vmn2r101 | 2.44E−11 |
| PEMN_1 | Gpr55 | 1.42E−09 |
| PEMN_1 | Mpped1 | 1.45E−09 |
| PEMN_1 | Pate4 | 4.30E−09 |
| PEMN_1 | Rdh8 | 1.10E−08 |
| PEMN_1 | Nostrin | 1.28E−08 |
| PEMN_1 | 5430427O19Rik | 1.64E−08 |
| PEMN_1 | Hapln4 | 5.31E−08 |
| PEMN_1 | 4933400B14Rik | 8.38E−08 |
| PEMN_1 | Serpinb3c | 8.92E−08 |
| PEMN_1 | Col9a1 | 9.03E−08 |
| PEMN_1 | Bhlha15 | 1.39E−07 |
| PEMN_1 | Lrtm1 | 1.46E−07 |
| PEMN_1 | Gm1631 | 2.36E−07 |
| PEMN_1 | Ptcra | 1.47E−06 |
| PEMN_1 | Gm5860 | 1.31E−05 |
| PEMN_1 | AA387883 | 1.32E−05 |
| PEMN_1 | Fgr | 1.45E−05 |
| PEMN_1 | Spc25 | 1.97E−05 |
| PEMN_1 | Gm11186 | 2.02E−05 |
| PEMN_1 | Cyp2c37 | 3.07E−05 |
| PEMN_1 | BC051628 | 3.28E−05 |
| PEMN_1 | Mmp12 | 1.14E−04 |
| PEMN_1 | Prlhr | 1.19E−04 |
| PEMN_1 | Gad1 | 1.33E−04 |
| PEMN_1 | Ptprv | 1.51E−04 |
| PEMN_1 | Ccdc108 | 1.77E−04 |
| PEMN_1 | Cldn18 | 1.80E−04 |
| PEMN_1 | Upk1b | 1.81E−04 |
| PEMN_1 | Ccna1 | 2.02E−04 |
| PEMN_1 | Ccdc113 | 2.34E−04 |
| PEMN_1 | Pvrl4 | 2.49E−04 |
| PEMN_1 | Ccdc154 | 3.21E−04 |
| PEMN_1 | Klf2 | 3.25E−04 |
| PEMN_1 | Itgb2l | 3.35E−04 |
| PEMN_1 | Ppp1r1c | 4.30E−04 |
| PEMN_1 | 1700064J06Rik | 4.78E−04 |
| PEMN_1 | Arhgap36 | 5.35E−04 |
| PEMN_1 | A230077H06Rik | 5.50E−04 |
| PEMN_1 | Cd180 | 5.60E−04 |
| PEMN_1 | Myf6 | 1.00E−03 |
| PEMN_1 | Gjc3 | 1.49E−03 |
| PEMN_1 | 1700006H21Rik | 1.65E−03 |
| PEMN_1 | Lrrc10b | 1.91E−03 |
| PEMN_1 | 1700112H15Rik | 1.97E−03 |
| PEMN_1 | A230001M10Rik | 2.98E−03 |
| PEMN_1 | BC125332 | 3.00E−03 |
| PEMN_1 | Bhmt | 3.04E−03 |
| PEMN_1 | Shisa3 | 3.40E−03 |
| PEMN_1 | Capn9 | 3.65E−03 |
| PEMN_1 | Foxj1 | 3.93E−03 |
| PEMN_1 | Trpa1 | 5.65E−03 |
| PEMN_1 | 4933425H06Rik | 5.97E−03 |
| PEMN_1 | Asb17 | 7.04E−03 |
| PEMN_1 | Tarm1 | 7.85E−03 |
| PEMN_1 | Prss29 | 8.05E−03 |
| PEMN_1 | Gpr33 | 9.93E−03 |
| PEMN_1 | Cmtm2a | 9.96E−03 |
| PEMN_1 | 7630403G23Rik | 1.13E−02 |
| PEMN_1 | Gpr52 | 1.26E−02 |
| PEMN_1 | Hs3st6 | 1.33E−02 |
| PEMN_1 | Ndufs5 | 1.34E−02 |
| PEMN_1 | Tmem154 | 1.36E−02 |
| PEMN_1 | Yipf7 | 1.49E−02 |
| PEMN_1 | Ribc2 | 1.52E−02 |
| PEMN_1 | H60c | 1.88E−02 |
| PEMN_1 | Vmn2r70 | 1.98E−02 |
| PEMN_1 | Rhoh | 2.07E−02 |
| PEMN_1 | 1700025F24Rik | 2.08E−02 |
| PEMN_1 | 1110059M19Rik | 2.60E−02 |
| PEMN_1 | Ttc24 | 2.90E−02 |
| PEMN_1 | Ecel1 | 3.07E−02 |
| PEMN_1 | P2ry13 | 3.21E−02 |
| PEMN_1 | Pinc | 3.36E−02 |
| PEMN_1 | Fmo6 | 4.06E−02 |
| PEMN_1 | 1700031A10Rik | 4.37E−02 |
| PEMN_1 | Dcaf12l2 | 4.48E−02 |
| PEMN_1 | Tmem81 | 4.76E−02 |
| PEMN_2 | Pgm5 | 1.56E−45 |
| PEMN_2 | Plxdc2 | 4.99E−44 |
| PEMN_2 | Edil3 | 8.16E−42 |
| PEMN_2 | Pion | 1.32E−35 |
| PEMN_2 | Kcns3 | 2.94E−35 |
| PEMN_2 | Lrp1b | 2.43E−34 |
| PEMN_2 | Gda | 1.41E−32 |
| PEMN_2 | Prom1 | 1.84E−32 |
| PEMN_2 | Extl1 | 4.61E−32 |
| PEMN_2 | Csmd3 | 6.31E−32 |
| PEMN_2 | Cntn3 | 1.05E−31 |
| PEMN_2 | Gria1 | 1.27E−28 |
| PEMN_2 | Rab3b | 6.77E−28 |
| PEMN_2 | Nxph1 | 2.64E−27 |
| PEMN_2 | Plcl1 | 3.61E−27 |
| PEMN_2 | Abca5 | 9.35E−27 |
| PEMN_2 | Shc4 | 5.90E−26 |
| PEMN_2 | Sphkap | 6.85E−26 |
| PEMN_2 | Vldlr | 1.39E−25 |
| PEMN_2 | Synpr | 2.99E−25 |
| PEMN_2 | Lrrc7 | 3.84E−25 |
| PEMN_2 | Tac1 | 1.35E−24 |
| PEMN_2 | Ccdc60 | 1.27E−23 |
| PEMN_2 | Agtr1a | 1.75E−23 |
| PEMN_2 | Cntn5 | 3.06E−23 |
| PEMN_2 | Prkg1 | 1.75E−22 |
| PEMN_2 | Pdlim3 | 1.43E−21 |
| PEMN_2 | Pde1b | 2.16E−21 |
| PEMN_2 | Crispld1 | 9.44E−21 |
| PEMN_2 | Lingo2 | 2.92E−20 |
| PEMN_2 | Dock10 | 5.93E−20 |
| PEMN_2 | Socs2 | 1.68E−19 |
| PEMN_2 | Cntnap5b | 5.11E−19 |
| PEMN_2 | Gas7 | 1.18E−18 |
| PEMN_2 | Kcnc2 | 1.83E−18 |
| PEMN_2 | Arhgap28 | 5.03E−18 |
| PEMN_2 | Srgap1 | 9.68E−17 |
| PEMN_2 | Ism1 | 1.34E−16 |
| PEMN_2 | Lin7a | 1.79E−16 |
| PEMN_2 | Rmst | 2.12E−16 |
| PEMN_2 | Grem2 | 3.69E−16 |
| PEMN_2 | Colq | 3.84E−16 |
| PEMN_2 | Kctd8 | 7.66E−16 |
| PEMN_2 | Lphn3 | 8.63E−16 |
| PEMN_2 | Fgfr2 | 1.07E−15 |
| PEMN_2 | Gpc6 | 1.25E−15 |
| PEMN_2 | Runx1t1 | 1.30E−15 |
| PEMN_2 | Olfm2 | 1.37E−15 |
| PEMN_2 | Fam19a5 | 1.82E−15 |
| PEMN_2 | Ryr2 | 1.83E−15 |
| PEMN_2 | Exoc3l4 | 2.46E−15 |
| PEMN_2 | Atp2b2 | 3.17E−15 |
| PEMN_2 | Ets1 | 3.17E−15 |
| PEMN_2 | A830018L16Rik | 3.53E−15 |
| PEMN_2 | Dmd | 3.53E−15 |
| PEMN_2 | Dach1 | 4.59E−15 |
| PEMN_2 | Unc5d | 4.62E−15 |
| PEMN_2 | Prkcb | 4.62E−15 |
| PEMN_2 | Il2 | 4.99E−15 |
| PEMN_2 | Calcrl | 1.21E−14 |
| PEMN_2 | Lsamp | 1.26E−14 |
| PEMN_2 | Ltbp4 | 3.55E−14 |
| PEMN_2 | Elavl2 | 7.97E−14 |
| PEMN_2 | Sh3rf3 | 1.32E−13 |
| PEMN_2 | Pld5 | 2.03E−13 |
| PEMN_2 | Tmem255a | 2.07E−13 |
| PEMN_2 | Cnr1 | 2.71E−13 |
| PEMN_2 | Ptprm | 2.71E−13 |
| PEMN_2 | Grik1 | 5.58E−13 |
| PEMN_2 | St8sia2 | 7.14E−13 |
| PEMN_2 | Casz1 | 1.10E−12 |
| PEMN_2 | Hgf | 1.19E−12 |
| PEMN_2 | Grm7 | 1.28E−12 |
| PEMN_2 | Gch1 | 1.94E−12 |
| PEMN_2 | Htr1f | 1.97E−12 |
| PEMN_2 | Stk32a | 2.31E−12 |
| PEMN_2 | Nkain2 | 2.47E−12 |
| PEMN_2 | Gucy1a3 | 2.76E−12 |
| PEMN_2 | Pmp22 | 5.40E−12 |
| PEMN_2 | Spock1 | 5.69E−12 |
| PEMN_2 | Slc16a12 | 5.96E−12 |
| PEMN_2 | Necab2 | 6.92E−12 |
| PEMN_2 | Whrn | 6.93E−12 |
| PEMN_2 | Nr1h4 | 7.65E−12 |
| PEMN_2 | Slc6a17 | 9.66E−12 |
| PEMN_2 | Camk4 | 1.04E−11 |
| PEMN_2 | Prmt8 | 1.09E−11 |
| PEMN_2 | Epb4.1l5 | 1.16E−11 |
| PEMN_2 | Sertm1 | 1.44E−11 |
| PEMN_2 | Gm15179 | 1.67E−11 |
| PEMN_2 | Trpc7 | 1.86E−11 |
| PEMN_2 | Gabrg3 | 1.90E−11 |
| PEMN_2 | Slit1 | 1.96E−11 |
| PEMN_2 | Msrb3 | 2.52E−11 |
| PEMN_2 | Ralyl | 2.54E−11 |
| PEMN_2 | Olfm1 | 2.86E−11 |
| PEMN_2 | Chst1 | 3.88E−11 |
| PEMN_2 | Diras2 | 5.82E−11 |
| PEMN_2 | Nyap2 | 5.84E−11 |
| PEMN_2 | Pcdh10 | 8.74E−11 |
| PEMN_2 | Corin | 2.93E−10 |
| PEMN_2 | Tmem252 | 6.73E−10 |
| PEMN_2 | Gm15080 | 1.18E−09 |
| PEMN_2 | 9830132P13Rik | 1.21E−09 |
| PEMN_2 | Adra1d | 1.30E−09 |
| PEMN_2 | Dbpht2 | 1.61E−09 |
| PEMN_2 | 1700027H10Rik | 1.07E−08 |
| PEMN_2 | Vmn2r105 | 1.93E−08 |
| PEMN_2 | Treml1 | 2.33E−08 |
| PEMN_2 | Nrsn2 | 4.30E−08 |
| PEMN_2 | Ene1 | 5.49E−08 |
| PEMN_2 | Trpm8 | 7.61E−08 |
| PEMN_2 | Prlhr | 1.12E−07 |
| PEMN_2 | Lypd1 | 1.77E−07 |
| PEMN_2 | 2010204K13Rik | 2.02E−07 |
| PEMN_2 | Cel | 2.06E−07 |
| PEMN_2 | Cst12 | 2.81E−07 |
| PEMN_2 | Gm11413 | 3.18E−07 |
| PEMN_2 | 1700109G14Rik | 5.03E−07 |
| PEMN_2 | Cpvl | 3.50E−06 |
| PEMN_2 | Klhdc8a | 4.14E−06 |
| PEMN_2 | Nox4 | 7.51E−06 |
| PEMN_2 | Mro | 2.64E−05 |
| PEMN_2 | Adm | 3.17E−05 |
| PEMN_2 | Olfr53 | 4.63E−05 |
| PEMN_2 | Emx2os | 4.81E−05 |
| PEMN_2 | Rxfp3 | 5.04E−05 |
| PEMN_2 | Bmx | 5.73E−05 |
| PEMN_2 | 7420701I03Rik | 7.05E−05 |
| PEMN_2 | Gm4340 | 7.54E−05 |
| PEMN_2 | Gata1 | 9.95E−05 |
| PEMN_2 | Zpld1 | 1.31E−04 |
| PEMN_2 | Clqtnf7 | 2.02E−04 |
| PEMN_2 | Alx1 | 2.12E−04 |
| PEMN_2 | Pdgfra | 2.89E−04 |
| PEMN_2 | Aurkb | 4.27E−04 |
| PEMN_2 | Psrc1 | 4.59E−04 |
| PEMN_2 | Hck | 7.42E−04 |
| PEMN_2 | 2310005A03Rik | 7.89E−04 |
| PEMN_2 | Cenpm | 9.08E−04 |
| PEMN_2 | Gabrd | 9.53E−04 |
| PEMN_2 | Apitd1 | 1.04E−03 |
| PEMN_2 | Fam84b | 1.05E−03 |
| PEMN_2 | Apobec2 | 1.72E−03 |
| PEMN_2 | Gdnf | 2.31E−03 |
| PEMN_2 | C330022C24Rik | 2.51E−03 |
| PEMN_2 | Tcl1b4 | 2.90E−03 |
| PEMN_2 | Gm14139 | 2.90E−03 |
| PEMN_2 | Tsga8 | 3.37E−03 |
| PEMN_2 | Hs3st3a1 | 3.59E−03 |
| PEMN_2 | Fcrl1 | 4.28E−03 |
| PEMN_2 | Gm11762 | 4.68E−03 |
| PEMN_2 | F730043M19Rik | 5.09E−03 |
| PEMN_2 | Krt76 | 6.59E−03 |
| PEMN_2 | Kel | 7.85E−03 |
| PEMN_2 | Klri1 | 8.17E−03 |
| PEMN_2 | Wbp2nl | 9.69E−03 |
| PEMN_2 | Rsg1 | 9.84E−03 |
| PEMN_2 | Rprm | 9.90E−03 |
| PEMN_2 | Tec | 1.03E−02 |
| PEMN_2 | 3110070M22Rik | 1.21E−02 |
| PEMN_2 | Gpr44 | 1.50E−02 |
| PEMN_2 | Gm4981 | 1.62E−02 |
| PEMN_2 | Il21 | 1.71E−02 |
| PEMN_2 | Wnt4 | 1.90E−02 |
| PEMN_2 | Wnt3a | 1.99E−02 |
| PEMN_2 | Plac1 | 2.05E−02 |
| PEMN_2 | 9230104L09Rik | 2.41E−02 |
| PEMN_2 | Pnma1 | 2.55E−02 |
| PEMN_2 | Cd3e | 2.70E−02 |
| PEMN_2 | Gm8298 | 2.72E−02 |
| PEMN_2 | Nmur1 | 2.72E−02 |
| PEMN_2 | Erg | 3.05E−02 |
| PEMN_2 | Ip6k3 | 3.45E−02 |
| PEMN_2 | Aqp12 | 3.98E−02 |
| PEMN_2 | Vmn2r68 | 4.01E−02 |
| PEMN_2 | 4933416M06Rik | 4.30E−02 |
| PEMN_2 | A630095N17Rik | 4.31E−02 |
| PEMN_2 | Alyref | 4.36E−02 |
| PEMN_2 | AA387883 | 4.55E−02 |
| PEMN_3 | Mir669a-7 | 1.23E−120 |
| PEMN_3 | Mir669a-5 | 1.15E−82 |
| PEMN_3 | Mir669a-10 | 4.21E−80 |
| PEMN_3 | Mir669p-1 | 1.37E−63 |
| PEMN_3 | Mir669a-4 | 5.53E−63 |
| PEMN_3 | Mir669a-6 | 3.11E−61 |
| PEMN_3 | Mir669a-8 | 9.12E−59 |
| PEMN_3 | Mir669a-11 | 1.26E−55 |
| PEMN_3 | Mir669p-2 | 9.48E−37 |
| PEMN_3 | Defb9 | 4.31E−29 |
| PEMN_3 | Mir669a-12 | 6.71E−29 |
| PEMN_3 | Astl | 1.05E−28 |
| PEMN_3 | Mir669a-9 | 1.95E−26 |
| PEMN_3 | Prss45 | 1.07E−25 |
| PEMN_3 | Ms4a6b | 1.82E−19 |
| PEMN_3 | Galp | 2.61E−19 |
| PEMN_3 | C5ar2 | 2.34E−17 |
| PEMN_3 | Siglec5 | 5.57E−16 |
| PEMN_3 | C330011F03Rik | 2.00E−12 |
| PEMN_3 | Gm17821 | l.00E−11 |
| PEMN_3 | Gm17830 | 1.73E−11 |
| PEMN_3 | Sult2a6 | 1.10E−10 |
| PEMN_3 | BC107364 | 6.26E−10 |
| PEMN_3 | AI504432 | 1.03E−08 |
| PEMN_3 | 1700066J24Rik | 1.66E−08 |
| PEMN_3 | Gm12603 | 2.10E−08 |
| PEMN_3 | 1700057G04Rik | 6.15E−08 |
| PEMN_3 | Ces2h | 8.26E−08 |
| PEMN_3 | Slc6a18 | 2.94E−07 |
| PEMN_3 | Dppa4 | 3.64E−07 |
| PEMN_3 | Plekhs1 | 7.18E−07 |
| PEMN_3 | Ddx43 | 7.18E−07 |
| PEMN_3 | Pabpc6 | 8.98E−07 |
| PEMN_3 | Sh3d21 | 1.03E−06 |
| PEMN_3 | Gm8801 | 1.44E−06 |
| PEMN_3 | Piwil4 | 1.44E−06 |
| PEMN_3 | C5ar1 | 6.17E−06 |
| PEMN_3 | 1700029F12Rik | 7.03E−06 |
| PEMN_3 | Fam78a | 1.69E−05 |
| PEMN_3 | Tada3 | 2.18E−05 |
| PEMN_3 | Traip | 6.43E−05 |
| PEMN_3 | Awat2 | 6.96E−05 |
| PEMN_3 | Lipm | 8.95E−05 |
| PEMN_3 | Vegfa | 2.74E−04 |
| PEMN_3 | Gm13544 | 2.92E−04 |
| PEMN_3 | Pramel4 | 4.74E−04 |
| PEMN_3 | D5Ertd577e | 4.97E−04 |
| PEMN_3 | Mrps7 | 6.01E−04 |
| PEMN_3 | 8030443G20Rik | 7.07E−04 |
| PEMN_3 | Rn4.5s | 8.46E−04 |
| PEMN_3 | Opn5 | 8.69E−04 |
| PEMN_3 | Olfr1 | 8.81E−04 |
| PEMN_3 | Nmral1 | 1.08E−03 |
| PEMN_3 | 9530080O11Rik | 1.08E−03 |
| PEMN_3 | Il13ra2 | 1.08E−03 |
| PEMN_3 | Vsig2 | 1.17E−03 |
| PEMN_3 | 2610318N02Rik | 1.20E−03 |
| PEMN_3 | Gm20337 | 1.27E−03 |
| PEMN_3 | 6030498E09Rik | 1.37E−03 |
| PEMN_3 | 2310034O05Rik | 1.38E−03 |
| PEMN_3 | Gm8363 | 1.56E−03 |
| PEMN_3 | Adra1a | 1.56E−03 |
| PEMN_3 | Kdm6b | 3.44E−03 |
| PEMN_3 | Iqgap3 | 3.63E−03 |
| PEMN_3 | Sec1 | 4.02E−03 |
| PEMN_3 | Fcrl5 | 4.03E−03 |
| PEMN_3 | Slc9c1 | 4.03E−03 |
| PEMN_3 | Cspg4 | 4.52E−03 |
| PEMN_3 | Nxt2 | 4.54E−03 |
| PEMN_3 | Trim30a | 4.55E−03 |
| PEMN_3 | 4930564G21Rik | 4.57E−03 |
| PEMN_3 | Pou2f2 | 4.96E−03 |
| PEMN_3 | Chrnb3 | 5.32E−03 |
| PEMN_3 | Btk | 5.35E−03 |
| PEMN_3 | Ccr4 | 5.81E−03 |
| PEMN_3 | Gramd1c | 6.66E−03 |
| PEMN_3 | Yipf7 | 7.24E−03 |
| PEMN_3 | Cyp2j5 | 7.57E−03 |
| PEMN_3 | Fat2 | 7.62E−03 |
| PEMN_3 | Gch1 | 8.05E−03 |
| PEMN_3 | Oscp1 | 8.16E−03 |
| PEMN_3 | Crisp2 | 8.62E−03 |
| PEMN_3 | Cxcr6 | 8.83E−03 |
| PEMN_3 | 9330133O14Rik | 1.01E−02 |
| PEMN_3 | Pbld1 | 1.01E−02 |
| PEMN_3 | Akip1 | 1.03E−02 |
| PEMN_3 | Gm5458 | 1.04E−02 |
| PEMN_3 | Lef1 | 1.04E−02 |
| PEMN_3 | Tmem132b | 1.11E−02 |
| PEMN_3 | Lsm3 | 1.21E−02 |
| PEMN_3 | Gm8267 | 1.21E−02 |
| PEMN_3 | Gm3258 | 1.32E−02 |
| PEMN_3 | Cpsf7 | 1.42E−02 |
| PEMN_3 | Zmym1 | 1.46E−02 |
| PEMN_3 | Slc25a41 | 1.48E−02 |
| PEMN_3 | 1700120C14Rik | 1.71E−02 |
| PEMN_3 | Nosip | 1.77E−02 |
| PEMN_3 | Mir568 | 1.85E−02 |
| PEMN_3 | Zfp106 | 2.07E−02 |
| PEMN_3 | Cyld | 2.07E−02 |
| PEMN_3 | Gprc6a | 2.20E−02 |
| PEMN_3 | Usp17le | 2.34E−02 |
| PEMN_3 | Sap30 | 2.34E−02 |
| PEMN_3 | Musk | 2.47E−02 |
| PEMN_3 | Olfr536 | 2.64E−02 |
| PEMN_3 | Klhdc8a | 2.64E−02 |
| PEMN_3 | Gm20187 | 3.12E−02 |
| PEMN_3 | Thpo | 3.27E−02 |
| PEMN_3 | Cytl1 | 3.32E−02 |
| PEMN_3 | Jag1 | 3.49E−02 |
| PEMN_3 | Lrrc71 | 3.51E−02 |
| PEMN_3 | 2010003O02Rik | 3.61E−02 |
| PEMN_3 | Laptm5 | 3.61E−02 |
| PEMN_3 | Vmn2r98 | 4.02E−02 |
| PEMN_3 | Tmc2 | 4.32E−02 |
| PEMN_3 | Tnfrsf11a | 4.32E−02 |
| PEMN_3 | Gm10058 | 4.36E−02 |
| PEMN_3 | Il1rl1 | 4.38E−02 |
| PEMN_3 | Cdc45 | 4.39E−02 |
| PEMN_3 | Gm20747 | 4.39E−02 |
| PEMN_3 | 1700008J07Rik | 4.49E−02 |
| PEMN_3 | Fsbp | 4.49E−02 |
| PEMN_3 | Zfp607 | 4.49E−02 |
| PEMN_3 | Raet1d | 4.70E−02 |
| PEMN_3 | Vmn2r44 | 4.71E−02 |
| PEMN_3 | Mira | 4.75E−02 |
| PEMN_3 | Alox12 | 4.85E−02 |
| PEMN_4 | Tmem132c | 7.91E−199 |
| PEMN_4 | Ptprt | 3.10E−189 |
| PEMN_4 | Grik1 | 8.96E−152 |
| PEMN_4 | Fbxw24 | 7.83E−123 |
| PEMN_4 | Plcxd3 | 2.04E−117 |
| PEMN_4 | Fam5b | 4.21E−115 |
| PEMN_4 | Cdc14a | 5.51E−114 |
| PEMN_4 | Sdk2 | 1.62E−111 |
| PEMN_4 | Tcf7l2 | 1.53E−108 |
| PEMN_4 | Arhgap24 | 4.76E−105 |
| PEMN_4 | Bnc2 | 4.52E−104 |
| PEMN_4 | Galnt14 | 2.15E−99 |
| PEMN_4 | Aik | 1.27E−98 |
| PEMN_4 | Caln1 | 1.98E−96 |
| PEMN_4 | Rbfox1 | 8.81E−95 |
| PEMN_4 | Satb1 | 6.50E−92 |
| PEMN_4 | Chat | 1.43E−91 |
| PEMN_4 | Adamts11 | 3.60E−91 |
| PEMN_4 | Fam19a1 | 6.15E−91 |
| PEMN_4 | Fgfr2 | 2.74E−90 |
| PEMN_4 | Fbxw15 | 5.92E−90 |
| PEMN_4 | Cacna1e | 6.65E−90 |
| PEMN_4 | Oprk1 | 3.14E−81 |
| PEMN_4 | Pi15 | 3.99E−81 |
| PEMN_4 | Wbscr17 | 6.40E−81 |
| PEMN_4 | Kalrn | 3.87E−80 |
| PEMN_4 | Tmem117 | 2.80E−76 |
| PEMN_4 | Ngef | 4.99E−73 |
| PEMN_4 | Ccbe1 | 2.08E−71 |
| PEMN_4 | St6galnac3 | 1.98E−70 |
| PEMN_4 | Casz1 | 3.90E−69 |
| PEMN_4 | Slc35f4 | 1.33E−68 |
| PEMN_4 | Fam19a2 | 6.33E−67 |
| PEMN_4 | Enox1 | 4.81E−66 |
| PEMN_4 | Pbx1 | 1.33E−64 |
| PEMN_4 | Fam19a5 | 8.03E−64 |
| PEMN_4 | Gm2694 | 4.13E−63 |
| PEMN_4 | Dlgap2 | 4.96E−63 |
| PEMN_4 | Fhit | 1.30E−62 |
| PEMN_4 | Pknox2 | 9.14E−62 |
| PEMN_4 | Bcar3 | 1.80E−61 |
| PEMN_4 | Gfra2 | 4.47E−61 |
| PEMN_4 | Prmt8 | 6.14E−59 |
| PEMN_4 | Pcdh7 | 7.14E−59 |
| PEMN_4 | Fam196b | 1.08E−58 |
| PEMN_4 | Col6a1 | 1.95E−58 |
| PEMN_4 | Slc26a4 | 3.65E−58 |
| PEMN_4 | Chsy3 | 1.21E−57 |
| PEMN_4 | Syn2 | 3.91E−57 |
| PEMN_4 | Gpc6 | 1.06E−56 |
| PEMN_4 | Fbln5 | 6.90E−56 |
| PEMN_4 | Pde4b | 3.14E−55 |
| PEMN_4 | Cd84 | 3.30E−54 |
| PEMN_4 | Sec16b | 3.49E−54 |
| PEMN_4 | Nfia | 1.76E−53 |
| PEMN_4 | Scube1 | 1.95E−53 |
| PEMN_4 | Fgd6 | 3.25E−52 |
| PEMN_4 | Dock2 | 4.17E−52 |
| PEMN_4 | Ly6e | 1.72E−51 |
| PEMN_4 | Xylt1 | 1.82E−51 |
| PEMN_4 | 1810041L15Rik | 2.67E−51 |
| PEMN_4 | Plod2 | 2.67E−51 |
| PEMN_4 | Dmkn | 5.72E−51 |
| PEMN_4 | Syt6 | 6.83E−51 |
| PEMN_4 | Piezo1 | 1.23E−50 |
| PEMN_4 | Chgb | 3.17E−50 |
| PEMN_4 | Ptpn5 | 1.11E−49 |
| PEMN_4 | Ghr | 1.11E−49 |
| PEMN_4 | Mdga1 | 2.52E−48 |
| PEMN_4 | Nfib | 3.73E−48 |
| PEMN_4 | Psd3 | 5.98E−48 |
| PEMN_4 | Cpne8 | 3.47E−47 |
| PEMN_4 | Elmo1 | 4.38E−47 |
| PEMN_4 | Pld5 | 2.61E−46 |
| PEMN_4 | Cyb561 | 2.69E−46 |
| PEMN_4 | Zfp521 | 4.91E−46 |
| PEMN_4 | Ebf3 | 5.51E−46 |
| PEMN_4 | Rspo2 | 1.54E−45 |
| PEMN_4 | 4933400C23Rik | 2.44E−45 |
| PEMN_4 | Dpyd | 2.60E−44 |
| PEMN_4 | Sulf2 | 1.68E−43 |
| PEMN_4 | Ppfibp1 | 1.75E−43 |
| PEMN_4 | Itgb5 | 4.39E−43 |
| PEMN_4 | Pdzrn4 | 8.06E−42 |
| PEMN_4 | Zbtb7c | 8.97E−42 |
| PEMN_4 | Igsf3 | 2.79E−41 |
| PEMN_4 | Tshz2 | 5.07E−41 |
| PEMN_4 | Lrig3 | 1.14E−40 |
| PEMN_4 | Tox | 4.45E−40 |
| PEMN_4 | Abcc8 | 7.29E−40 |
| PEMN_4 | 1700123O21Rik | 1.59E−39 |
| PEMN_4 | Peli2 | 2.58E−39 |
| PEMN_4 | Itga6 | 4.51E−39 |
| PEMN_4 | Sgpp2 | 1.51E−38 |
| PEMN_4 | Scg2 | 2.61E−38 |
| PEMN_4 | Cyyr1 | 1.21E−37 |
| PEMN_4 | Gpm6b | 3.06E−37 |
| PEMN_4 | B3gat1 | 1.17E−36 |
| PEMN_4 | 1700085B03Rik | 3.96E−36 |
| PEMN_4 | Ppapdc1a | 7.01E−36 |
| PEMN_4 | Cxcl12 | 2.71E−34 |
| PEMN_4 | Drd2 | 2.88E−34 |
| PEMN_4 | Sntg2 | 6.81E−32 |
| PEMN_4 | Kcns2 | 3.80E−28 |
| PEMN_4 | Dsc3 | 1.01E−26 |
| PEMN_4 | Cldn8 | 3.00E−25 |
| PEMN_4 | Fbxw16 | 2.35E−23 |
| PEMN_4 | Zfp185 | 2.88E−23 |
| PEMN_4 | Heg1 | 6.07E−23 |
| PEMN_4 | Itga4 | 9.57E−21 |
| PEMN_4 | Cacng3 | 3.61E−20 |
| PEMN_4 | Hsd3b6 | 1.25E−17 |
| PEMN_4 | Plekhd1 | 2.78E−17 |
| PEMN_4 | Cbln1 | 5.01E−17 |
| PEMN_4 | Ahsg | 9.32E−17 |
| PEMN_4 | Mn1 | 3.22E−15 |
| PEMN_4 | Rgcc | 8.72E−14 |
| PEMN_4 | Bpifb4 | 6.26E−13 |
| PEMN_4 | Ly6c1 | 8.21E−13 |
| PEMN_4 | Aldh3a1 | 3.80E−12 |
| PEMN_4 | Entpd2 | 6.52E−12 |
| PEMN_4 | Ces2g | 7.64E−12 |
| PEMN_4 | Tnnt2 | 5.60E−11 |
| PEMN_4 | Sardh | 7.15E−11 |
| PEMN_4 | 4632428N05Rik | 4.71E−10 |
| PEMN_4 | Gabra4 | 6.39E−10 |
| PEMN_4 | Fam83a | 7.00E−10 |
| PEMN_4 | Crispld2 | 1.46E−09 |
| PEMN_4 | Krt79 | 3.13E−09 |
| PEMN_4 | Aldh1a7 | 4.68E−08 |
| PEMN_4 | Megf6 | 9.24E−08 |
| PEMN_4 | Chst5 | 1.40E−07 |
| PEMN_4 | C330008G21Rik | 2.49E−07 |
| PEMN_4 | 1700007K13Rik | 3.42E−05 |
| PEMN_4 | Prdm12 | 3.42E−05 |
| PEMN_4 | Ndufa4l2 | 4.20E−05 |
| PEMN_4 | Ubxn10 | 5.53E−05 |
| PEMN_4 | Gm6455 | 9.45E−05 |
| PEMN_4 | Il7r | 1.78E−04 |
| PEMN_4 | Psg25 | 2.23E−04 |
| PEMN_4 | Klra6 | 2.75E−04 |
| PEMN_4 | Fetub | 6.31E−04 |
| PEMN_4 | Ang3 | 7.43E−04 |
| PEMN_4 | Ang5 | 7.44E−04 |
| PEMN_4 | Klra19 | 8.59E−04 |
| PEMN_4 | Cplx3 | 1.05E−03 |
| PEMN_4 | Gm10787 | 1.75E−03 |
| PEMN_4 | Cyp27b1 | 2.64E−03 |
| PEMN_4 | Slcl7a1 | 4.07E−03 |
| PEMN_4 | Mup19 | 4.37E−03 |
| PEMN_4 | Pla2g2f | 4.42E−03 |
| PEMN_4 | 4930529C04Rik | 4.92E−03 |
| PEMN_4 | 5430427M07Rik | 5.03E−03 |
| PEMN_4 | Olfr283 | 5.06E−03 |
| PEMN_4 | Acap1 | 6.19E−03 |
| PEMN_4 | C130074G19Rik | 6.53E−03 |
| PEMN_4 | Ctsq | 8.02E−03 |
| PEMN_4 | 1700023F02Rik | 8.37E−03 |
| PEMN_4 | Comp | 8.63E−03 |
| PEMN_4 | 4930433N12Rik | 1.06E−02 |
| PEMN_4 | Lefty2 | 1.22E−02 |
| PEMN_4 | Kif2c | 1.71E−02 |
| PEMN_4 | Adam28 | 1.82E−02 |
| PEMN_4 | Slc22a26 | 1.88E−02 |
| PEMN_4 | Gsta2 | 1.94E−02 |
| PEMN_4 | 1700003H04Rik | 2.18E−02 |
| PEMN_4 | Gm5105 | 2.46E−02 |
| PEMN_4 | Myh8 | 2.53E−02 |
| PEMN_4 | Gm11190 | 2.95E−02 |
| PEMN_4 | Ccl21b | 3.57E−02 |
| PEMN_4 | Chrna9 | 4.02E−02 |
| PEMN_4 | Odf3l1 | 4.16E−02 |
| PEMN_4 | Strc | 4.19E−02 |
| PEMN_4 | BC018473 | 4.26E−02 |
| PEMN_4 | Gm13807 | 4.26E−02 |
| PEMN_4 | Sim2 | 4.33E−02 |
| PEMN_4 | Slc10a5 | 4.38E−02 |
| PEMN_4 | Gm5797 | 4.59E−02 |
| PEMN_4 | Sp6 | 4.65E−02 |
| PEMN_5 | Oprk1 | 1.40E−59 |
| PEMN_5 | Aik | 2.34E−57 |
| PEMN_5 | Galntl6 | 2.39E−57 |
| PEMN_5 | Nkain2 | 8.20E−56 |
| PEMN_5 | Ptprt | 4.60E−55 |
| PEMN_5 | Fgfr2 | 7.19E−53 |
| PEMN_5 | Prmt8 | 1.19E−51 |
| PEMN_5 | Grik1 | 1.04E−49 |
| PEMN_5 | Pde4b | 9.32E−49 |
| PEMN_5 | Pld5 | 5.80E−47 |
| PEMN_5 | Sdk2 | 6.56E−47 |
| PEMN_5 | Adamts11 | 7.79E−46 |
| PEMN_5 | Plscr2 | 3.65E−44 |
| PEMN_5 | Bnc2 | 9.74E−44 |
| PEMN_5 | Satb1 | 1.40E−43 |
| PEMN_5 | Colq | 1.40E−42 |
| PEMN_5 | Ubash3b | 3.70E−42 |
| PEMN_5 | Tac1 | 1.40E−38 |
| PEMN_5 | Tmem163 | 2.38E−38 |
| PEMN_5 | Gucy1a3 | 5.02E−38 |
| PEMN_5 | Casz1 | 5.66E−37 |
| PEMN_5 | Gfra2 | 1.46E−36 |
| PEMN_5 | Syt6 | 2.15E−35 |
| PEMN_5 | Rab3b | 4.17E−35 |
| PEMN_5 | Pcdh7 | 5.37E−35 |
| PEMN_5 | Chat | 1.33E−34 |
| PEMN_5 | St6galnac3 | 3.58E−34 |
| PEMN_5 | Arhgap24 | 5.06E−34 |
| PEMN_5 | Elfn1 | 7.60E−34 |
| PEMN_5 | Trpc7 | 8.58E−34 |
| PEMN_5 | Gm5535 | 1.29E−33 |
| PEMN_5 | Fam19a5 | 5.15E−33 |
| PEMN_5 | Unc5d | 6.27E−33 |
| PEMN_5 | Dmkn | 6.89E−33 |
| PEMN_5 | Plod2 | 9.61E−33 |
| PEMN_5 | Tpd52l1 | 1.81E−32 |
| PEMN_5 | Cntnap5b | 2.15E−32 |
| PEMN_5 | Sulf2 | 3.76E−32 |
| PEMN_5 | Synpr | 3.97E−32 |
| PEMN_5 | Ralyl | 5.55E−32 |
| PEMN_5 | Fam19a1 | 6.66E−32 |
| PEMN_5 | 1810041L15Rik | 1.91E−31 |
| PEMN_5 | Sphkap | 4.51E−31 |
| PEMN_5 | Prickle2 | 5.70E−31 |
| PEMN_5 | Cd44 | 1.49E−30 |
| PEMN_5 | Rbfox1 | 1.49E−30 |
| PEMN_5 | Plcxd3 | 2.35E−30 |
| PEMN_5 | Kctd8 | 2.39E−30 |
| PEMN_5 | Cdh13 | 8.59E−30 |
| PEMN_5 | Gm2694 | 1.48E−29 |
| PEMN_5 | Ddr2 | 1.50E−29 |
| PEMN_5 | Zbtb16 | 2.71E−29 |
| PEMN_5 | Lingo2 | 1.22E−28 |
| PEMN_5 | Ust | 1.65E−28 |
| PEMN_5 | Epha7 | 2.01E−28 |
| PEMN_5 | Grm7 | 2.42E−28 |
| PEMN_5 | Zbtb7c | 9.91E−28 |
| PEMN_5 | Tmem117 | 1.24E−27 |
| PEMN_5 | Slc5a7 | 2.28E−27 |
| PEMN_5 | Mdga1 | 9.45E−27 |
| PEMN_5 | Colec12 | 3.07E−26 |
| PEMN_5 | Calcrl | 7.82E−26 |
| PEMN_5 | Bcar3 | 8.49E−26 |
| PEMN_5 | Abtb2 | 1.37E−25 |
| PEMN_5 | Kalrn | 1.54E−25 |
| PEMN_5 | 6330403A02Rik | 1.68E−25 |
| PEMN_5 | Abcc8 | 2.23E−25 |
| PEMN_5 | Usp6nl | 2.90E−25 |
| PEMN_5 | Prkcb | 3.02E−25 |
| PEMN_5 | Unc5c | 4.04E−25 |
| PEMN_5 | VIdlr | 9.29E−25 |
| PEMN_5 | Gpc6 | 1.40E−24 |
| PEMN_5 | Gch1 | 1.40E−24 |
| PEMN_5 | Dpyd | 2.67E−24 |
| PEMN_5 | Frmd4b | 4.78E−24 |
| PEMN_5 | Itga6 | 5.11E−24 |
| PEMN_5 | Meis1 | 1.50E−23 |
| PEMN_5 | Lrp1b | 1.63E−23 |
| PEMN_5 | Htr4 | 1.64E−23 |
| PEMN_5 | Stxbp5l | 3.56E−23 |
| PEMN_5 | Tshz2 | 3.80E−23 |
| PEMN_5 | Ptprd | 4.82E−23 |
| PEMN_5 | Plscr4 | 9.52E−23 |
| PEMN_5 | Syn2 | 2.34E−22 |
| PEMN_5 | Ccdc60 | 4.55E−22 |
| PEMN_5 | Npy1r | 7.12E−22 |
| PEMN_5 | Grip1 | 7.35E−22 |
| PEMN_5 | Ltbp4 | 9.97E−22 |
| PEMN_5 | Neat1 | 9.97E−22 |
| PEMN_5 | Lrrc7 | 1.08E−21 |
| PEMN_5 | Nyap2 | 1.08E−21 |
| PEMN_5 | Syt1 | 1.17E−21 |
| PEMN_5 | Ryr1 | 1.27E−21 |
| PEMN_5 | Col4a2 | 1.52E−21 |
| PEMN_5 | Nxph1 | 2.92E−21 |
| PEMN_5 | Fam117a | 4.95E−21 |
| PEMN_5 | Tox | 7.17E−21 |
| PEMN_5 | Slc26a4 | 1.05E−20 |
| PEMN_5 | Slit1 | 1.17E−20 |
| PEMN_5 | Slc6a17 | 1.78E−20 |
| PEMN_5 | Gm15881 | 4.79E−20 |
| PEMN_5 | BC030500 | 1.10E−19 |
| PEMN_5 | Adrb2 | 1.42E−18 |
| PEMN_5 | 9530026F06Rik | 6.31E−16 |
| PEMN_5 | Ffar3 | 5.40E−14 |
| PEMN_5 | Cnih3 | 4.98E−13 |
| PEMN_5 | Cldn8 | 6.27E−13 |
| PEMN_5 | Adamts12 | 3.83E−12 |
| PEMN_5 | Fam19a3 | 6.00E−11 |
| PEMN_5 | Rgcc | 1.58E−10 |
| PEMN_5 | Hs3st4 | 3.01E−10 |
| PEMN_5 | Pthlh | 1.68E−09 |
| PEMN_5 | Prl2c5 | 2.69E−09 |
| PEMN_5 | Gm10637 | 1.22E−08 |
| PEMN_5 | Gm4791 | 2.60E−08 |
| PEMN_5 | Adamts14 | 3.45E−08 |
| PEMN_5 | Tmem92 | 2.10E−07 |
| PEMN_5 | Vwa2 | 7.37E−07 |
| PEMN_5 | Pdcd1 | 9.79E−07 |
| PEMN_5 | 4930539C22Rik | 1.07E−06 |
| PEMN_5 | Sprr2d | 1.58E−06 |
| PEMN_5 | 1700029H14Rik | 4.21E−05 |
| PEMN_5 | Defb1 | 4.26E−05 |
| PEMN_5 | Hsd17b13 | 7.82E−05 |
| PEMN_5 | BC030867 | 8.84E−05 |
| PEMN_5 | Ccdc153 | 8.93E−05 |
| PEMN_5 | Ccr4 | 1.17E−04 |
| PEMN_5 | Cyp4f18 | 1.25E−04 |
| PEMN_5 | Grasp | 1.33E−04 |
| PEMN_5 | Acan | 1.41E−04 |
| PEMN_5 | 6030419C18Rik | 1.42E−04 |
| PEMN_5 | Fli1 | 2.11E−04 |
| PEMN_5 | Tspan11 | 2.45E−04 |
| PEMN_5 | 4930479D17Rik | 2.52E−04 |
| PEMN_5 | Folr1 | 3.79E−04 |
| PEMN_5 | Fxyd2 | 3.99E−04 |
| PEMN_5 | Cyp3a59 | 5.04E−04 |
| PEMN_5 | Ifitm1 | 5.34E−04 |
| PEMN_5 | Tctex1d4 | 7.73E−04 |
| PEMN_5 | Cd209a | 7.89E−04 |
| PEMN_5 | Gm5168 | 8.36E−04 |
| PEMN_5 | 1700073E17Rik | 9.36E−04 |
| PEMN_5 | Gm20187 | 9.62E−04 |
| PEMN_5 | Myl10 | 1.07E−03 |
| PEMN_5 | 4930567H12Rik | 1.09E−03 |
| PEMN_5 | 4930438E09Rik | 1.21E−03 |
| PEMN_5 | Slc38a3 | 1.24E−03 |
| PEMN_5 | A530050N04Rik | 1.30E−03 |
| PEMN_5 | Vmn2r106 | 1.36E−03 |
| PEMN_5 | Cst12 | 1.92E−03 |
| PEMN_5 | Ffar2 | 2.84E−03 |
| PEMN_5 | Slc51b | 2.86E−03 |
| PEMN_5 | 4933407G14Rik | 3.09E−03 |
| PEMN_5 | Krt79 | 3.86E−03 |
| PEMN_5 | Pyhin1 | 4.17E−03 |
| PEMN_5 | Hist1h2an | 4.33E−03 |
| PEMN_5 | Gzmf | 4.81E−03 |
| PEMN_5 | Tmprss3 | 6.35E−03 |
| PEMN_5 | 1700065J18Rik | 6.88E−03 |
| PEMN_5 | Nxnl2 | 7.17E−03 |
| PEMN_5 | Gm4956 | 7.59E−03 |
| PEMN_5 | Pga5 | 8.53E−03 |
| PEMN_5 | Xlr5c | 9.75E−03 |
| PEMN_5 | Gm9866 | 1.17E−02 |
| PEMN_5 | 4930455B14Rik | 1.21E−02 |
| PEMN_5 | Tfap2c | 1.25E−02 |
| PEMN_5 | Lacc1 | 1.33E−02 |
| PEMN_5 | Samsn1 | 1.70E−02 |
| PEMN_5 | 1700065D16Rik | 1.86E−02 |
| PEMN_5 | Zbtb42 | 1.95E−02 |
| PEMN_5 | Ptafr | 2.15E−02 |
| PEMN_5 | AI747448 | 2.24E−02 |
| PEMN_5 | Wnt8a | 2.28E−02 |
| PEMN_5 | Cebpe | 2.55E−02 |
| PEMN_5 | Olfr1157 | 2.62E−02 |
| PEMN_5 | Lrit3 | 2.65E−02 |
| PEMN_5 | Rtp2 | 2.66E−02 |
| PEMN_5 | Mir22 | 2.73E−02 |
| PEMN_5 | Serpinb8 | 2.73E−02 |
| PEMN_5 | Pgf | 2.88E−02 |
| PEMN_5 | Ctrb1 | 3.00E−02 |
| PEMN_5 | 4930487D11Rik | 3.03E−02 |
| PEMN_5 | Ttc34 | 3.44E−02 |
| PEMN_5 | 2310014L17Rik | 3.52E−02 |
| PEMN_5 | 3110045C21Rik | 3.55E−02 |
| PEMN_5 | Bhmt2 | 4.12E−02 |
| PEMN_5 | 4833412C05Rik | 4.12E−02 |
| PEMN_5 | Pira2 | 4.43E−02 |
| PEMN_5 | Hsd3b1 | 4.47E−02 |
| PEMN_5 | Myoz1 | 4.48E−02 |
| PEMN_5 | Serpinb9g | 4.78E−02 |
| PEMN_6 | Oprk1 | 3.46E−78 |
| PEMN_6 | Galntl6 | 1.75E−67 |
| PEMN_6 | Epha6 | 2.00E−56 |
| PEMN_6 | Lrp1b | 3.65E−56 |
| PEMN_6 | Csmd3 | 5.09E−56 |
| PEMN_6 | Usp6nl | 9.60E−55 |
| PEMN_6 | Cd44 | 1.59E−54 |
| PEMN_6 | Nxph1 | 1.43E−53 |
| PEMN_6 | Cdh18 | 4.47E−52 |
| PEMN_6 | Tac1 | 2.85E−49 |
| PEMN_6 | Grik1 | 5.16E−48 |
| PEMN_6 | St6galnac3 | 2.75E−47 |
| PEMN_6 | Fgfr2 | 1.31E−46 |
| PEMN_6 | Hgf | 1.90E−46 |
| PEMN_6 | Antxr2 | 2.41E−46 |
| PEMN_6 | Pld5 | 1.50E−45 |
| PEMN_6 | Tpd52l1 | 2.19E−45 |
| PEMN_6 | Car10 | 3.72E−44 |
| PEMN_6 | Agtr1a | 4.13E−42 |
| PEMN_6 | Elfn1 | 7.48E−41 |
| PEMN_6 | Gda | 4.80E−40 |
| PEMN_6 | Spock1 | 2.27E−39 |
| PEMN_6 | Col6a1 | 2.83E−39 |
| PEMN_6 | Mir669b | 2.54E−38 |
| PEMN_6 | Gucy1a3 | 2.78E−38 |
| PEMN_6 | Kctd8 | 4.93E−38 |
| PEMN_6 | Aik | 5.07E−37 |
| PEMN_6 | Rftn1 | 7.24E−37 |
| PEMN_6 | Rhox2a | 7.67E−37 |
| PEMN_6 | Unc5d | 2.65E−36 |
| PEMN_6 | Plscr2 | 3.06E−36 |
| PEMN_6 | Colec12 | 1.09E−35 |
| PEMN_6 | Col6a2 | 3.04E−35 |
| PEMN_6 | Lrrc7 | 3.73E−34 |
| PEMN_6 | Satb1 | 3.95E−34 |
| PEMN_6 | Dlgap2 | 1.12E−33 |
| PEMN_6 | Pi15 | 3.56E−33 |
| PEMN_6 | Bnc2 | 1.60E−32 |
| PEMN_6 | Ralyl | 4.07E−32 |
| PEMN_6 | Colq | 5.33E−32 |
| PEMN_6 | Fstl4 | 5.34E−32 |
| PEMN_6 | Ccdc60 | 5.78E−32 |
| PEMN_6 | Gfra2 | 8.50E−32 |
| PEMN_6 | Slit1 | 2.18E−31 |
| PEMN_6 | Prickle2 | 2.41E−31 |
| PEMN_6 | Gpc6 | 4.29E−31 |
| PEMN_6 | Bai3 | 4.74E−31 |
| PEMN_6 | Epha7 | 6.61E−30 |
| PEMN_6 | Meis1 | 7.11E−30 |
| PEMN_6 | Prkcb | 1.65E−29 |
| PEMN_6 | Fam19a5 | 3.01E−29 |
| PEMN_6 | Fam5b | 5.74E−29 |
| PEMN_6 | Htr4 | 6.84E−29 |
| PEMN_6 | Dnahc5 | 9.07E−29 |
| PEMN_6 | Rgs20 | 2.68E−28 |
| PEMN_6 | Cpne8 | 4.28E−28 |
| PEMN_6 | Sphkap | 4.96E−28 |
| PEMN_6 | Ltbp4 | 5.23E−28 |
| PEMN_6 | Cntn3 | 5.23E−28 |
| PEMN_6 | 2610316D01Rik | 5.85E−28 |
| PEMN_6 | Slc5a7 | 1.15E−27 |
| PEMN_6 | Agbl4 | 1.18E−27 |
| PEMN_6 | Chat | 5.80E−27 |
| PEMN_6 | Parvb | 7.63E−27 |
| PEMN_6 | Ets1 | 1.84E−26 |
| PEMN_6 | Cntnap5b | 2.47E−26 |
| PEMN_6 | Kcnma1 | 3.24E−26 |
| PEMN_6 | Ryr3 | 1.37E−25 |
| PEMN_6 | Hs3st4 | 3.17E−25 |
| PEMN_6 | Pcdh7 | 5.19E−25 |
| PEMN_6 | Fstl5 | 5.59E−25 |
| PEMN_6 | Tmem117 | 1.14E−24 |
| PEMN_6 | Slc16a12 | 1.22E−24 |
| PEMN_6 | Calcrl | 1.69E−24 |
| PEMN_6 | Rbfox1 | 1.87E−24 |
| PEMN_6 | Ghr | 2.02E−24 |
| PEMN_6 | Fam196b | 2.26E−24 |
| PEMN_6 | Specc1 | 3.62E−24 |
| PEMN_6 | Casz1 | 6.72E−24 |
| PEMN_6 | Grip1 | 8.18E−24 |
| PEMN_6 | Nkain2 | 8.72E−24 |
| PEMN_6 | Pde4b | 1.58E−23 |
| PEMN_6 | Col5a3 | 2.00E−23 |
| PEMN_6 | Lingo2 | 3.27E−23 |
| PEMN_6 | Pgm5 | 4.71E−23 |
| PEMN_6 | A830018L16Rik | 4.73E−23 |
| PEMN_6 | Necab2 | 5.77E−23 |
| PEMN_6 | Cntnap2 | 9.64E−23 |
| PEMN_6 | Lemd1 | 1.18E−22 |
| PEMN_6 | Pdlim3 | 1.26E−22 |
| PEMN_6 | Tox | 1.50E−22 |
| PEMN_6 | Slc6a17 | 1.68E−22 |
| PEMN_6 | Tmem163 | 2.60E−22 |
| PEMN_6 | Sntg2 | 4.50E−22 |
| PEMN_6 | Slc24a4 | 1.22E−21 |
| PEMN_6 | Dcbld2 | 1.27E−21 |
| PEMN_6 | Nrgn | 2.80E−21 |
| PEMN_6 | Sdk2 | 2.80E−21 |
| PEMN_6 | Rfx3 | 3.02E−21 |
| PEMN_6 | Olfm3 | 3.95E−21 |
| PEMN_6 | Gm13034 | 2.10E−17 |
| PEMN_6 | 4933407I05Rik | 7.43E−14 |
| PEMN_6 | Mocs3 | 4.11E−11 |
| PEMN_6 | Arhgef39 | 5.12E−11 |
| PEMN_6 | 6530411M01Rik | 2.57E−10 |
| PEMN_6 | Card11 | 2.93E−09 |
| PEMN_6 | Apbb1ip | 1.71E−08 |
| PEMN_6 | C030034L19Rik | 1.41E−07 |
| PEMN_6 | Ghrh | 2.08E−07 |
| PEMN_6 | Habp2 | 1.43E−06 |
| PEMN_6 | Il23a | 2.50E−06 |
| PEMN_6 | Obp1a | 4.13E−06 |
| PEMN_6 | Gm6936 | 4.16E−06 |
| PEMN_6 | 8430422H06Rik | 4.95E−06 |
| PEMN_6 | Cyp4f39 | 1.08E−05 |
| PEMN_6 | Dct | 1.36E−05 |
| PEMN_6 | Ace2 | 1.55E−05 |
| PEMN_6 | Ace3 | 2.58E−05 |
| PEMN_6 | Galnt15 | 3.21E−05 |
| PEMN_6 | Grpr | 4.35E−05 |
| PEMN_6 | Serpinf2 | 4.35E−05 |
| PEMN_6 | Btnl1 | 7.72E−05 |
| PEMN_6 | Esm1 | 9.57E−05 |
| PEMN_6 | Mogat1 | 1.14E−04 |
| PEMN_6 | Dsg1b | 1.36E−04 |
| PEMN_6 | Ndnf | 1.90E−04 |
| PEMN_6 | A830019L24Rik | 2.09E−04 |
| PEMN_6 | Gm14858 | 2.19E−04 |
| PEMN_6 | Rfx8 | 3.63E−04 |
| PEMN_6 | 4933405O20Rik | 3.84E−04 |
| PEMN_6 | Sema7a | 4.92E−04 |
| PEMN_6 | Oxgr1 | 5.51E−04 |
| PEMN_6 | 1700034K08Rik | 6.66E−04 |
| PEMN_6 | 4933407L21Rik | 7.46E−04 |
| PEMN_6 | Cpa4 | 7.46E−04 |
| PEMN_6 | BC055111 | 7.90E−04 |
| PEMN_6 | Gm14635 | 7.90E−04 |
| PEMN_6 | Nmur1 | 9.80E−04 |
| PEMN_6 | Akr1cl | 1.09E−03 |
| PEMN_6 | BC090627 | 1.17E−03 |
| PEMN_6 | 1700007K09Rik | 1.67E−03 |
| PEMN_6 | Cyp2b10 | 1.92E−03 |
| PEMN_6 | Snai1 | 1.96E−03 |
| PEMN_6 | Tat | 2.06E−03 |
| PEMN_6 | Grm2 | 2.28E−03 |
| PEMN_6 | Gm6260 | 2.58E−03 |
| PEMN_6 | Ctsm | 2.71E−03 |
| PEMN_6 | 4930438E09Rik | 2.77E−03 |
| PEMN_6 | Htr5b | 3.09E−03 |
| PEMN_6 | Dsg1a | 3.52E−03 |
| PEMN_6 | Crisp1 | 3.95E−03 |
| PEMN_6 | Gimap3 | 5.82E−03 |
| PEMN_6 | Stc1 | 6.16E−03 |
| PEMN_6 | Tmco2 | 6.26E−03 |
| PEMN_6 | Gm11110 | 7.00E−03 |
| PEMN_6 | C86695 | 7.06E−03 |
| PEMN_6 | Batf | 8.19E−03 |
| PEMN_6 | Tcl1b1 | 8.20E−03 |
| PEMN_6 | Gm5077 | 1.06E−02 |
| PEMN_6 | Prl2c1 | 1.07E−02 |
| PEMN_6 | Il21 | 1.38E−02 |
| PEMN_6 | Alx3 | 1.79E−02 |
| PEMN_6 | Peg10 | 1.94E−02 |
| PEMN_6 | Neu2 | 2.25E−02 |
| PEMN_6 | Mettl11b | 2.38E−02 |
| PEMN_6 | Tnfrsf26 | 2.50E−02 |
| PEMN_6 | Kcnj10 | 2.58E−02 |
| PEMN_6 | Fgb | 2.60E−02 |
| PEMN_6 | LOC171588 | 2.64E−02 |
| PEMN_6 | Gm3776 | 2.71E−02 |
| PEMN_6 | Gsdma | 2.71E−02 |
| PEMN_6 | Ftcd | 2.86E−02 |
| PEMN_6 | 1700003F12Rik | 2.89E−02 |
| PEMN_6 | 5830403L16Rik | 2.94E−02 |
| PEMN_6 | F2rl1 | 2.97E−02 |
| PEMN_6 | Usp51 | 3.16E−02 |
| PEMN_6 | Tlr5 | 3.20E−02 |
| PEMN_6 | Sec14l4 | 3.44E−02 |
| PEMN_6 | Hes3 | 3.57E−02 |
| PEMN_6 | Gm11186 | 3.97E−02 |
| PEMN_6 | H2-Ob | 4.17E−02 |
| PEMN_6 | Nek2 | 4.57E−02 |
| PEMN_6 | Psg28 | 4.60E−02 |
| PEMN_6 | Susd3 | 4.85E−02 |
| PEMN_6 | Vax2os | 4.93E−02 |
| PIMN_1 | Cdh20 | 9.96E−140 |
| PIMN_1 | Rgs22 | 2.59E−111 |
| PIMN_1 | Syn3 | 8.57E−105 |
| PIMN_1 | Nos1 | 5.17E−103 |
| PIMN_1 | Timp3 | 2.42E−96 |
| PIMN_1 | 1700113H08Rik | 8.42E−94 |
| PIMN_1 | Fam65b | 8.26E−93 |
| PIMN_1 | Adcy2 | 9.99E−90 |
| PIMN_1 | Aldh1a3 | 7.02E−89 |
| PIMN_1 | Htr2c | 5.23E−88 |
| PIMN_1 | Pde1c | 6.69E−87 |
| PIMN_1 | Alcam | 3.24E−84 |
| PIMN_1 | Stxbp6 | 1.95E−80 |
| PIMN_1 | Fat3 | 1.54E−79 |
| PIMN_1 | Kirrel3 | 7.96E−79 |
| PIMN_1 | Stab2 | 8.53E−79 |
| PIMN_1 | Vwa5b1 | 2.79E−78 |
| PIMN_1 | Col5a2 | 1.23E−74 |
| PIMN_1 | Slco3a1 | 4.23E−74 |
| PIMN_1 | Cntnap5a | 2.33E−73 |
| PIMN_1 | Fbxo7 | 3.80E−73 |
| PIMN_1 | Rora | 4.71E−73 |
| PIMN_1 | Rnf144b | 5.06E−72 |
| PIMN_1 | St18 | 5.20E−72 |
| PIMN_1 | Zfp536 | 8.90E−72 |
| PIMN_1 | Gfra1 | 5.11E−70 |
| PIMN_1 | Epha5 | 1.04E−69 |
| PIMN_1 | Oprd1 | 1.24E−69 |
| PIMN_1 | Slc35f1 | 7.07E−69 |
| PIMN_1 | Aebp1 | 8.86E−69 |
| PIMN_1 | Cacnb2 | 6.04E−67 |
| PIMN_1 | Plxnb1 | 1.48E−65 |
| PIMN_1 | Enpp1 | 1.32E−63 |
| PIMN_1 | Dgkb | 1.32E−63 |
| PIMN_1 | Fam155a | 2.04E−63 |
| PIMN_1 | Col25a1 | 2.71E−60 |
| PIMN_1 | Pde1a | 9.57E−60 |
| PIMN_1 | Lrrc4c | 1.06E−59 |
| PIMN_1 | Etv1 | 4.15E−59 |
| PIMN_1 | Cd1d1 | 6.07E−57 |
| PIMN_1 | Arhgap15 | 1.21E−56 |
| PIMN_1 | Cadps2 | 2.68E−56 |
| PIMN_1 | Dach2 | 3.09E−56 |
| PIMN_1 | Entpd3 | 2.76E−55 |
| PIMN_1 | Kcnab1 | 4.67E−55 |
| PIMN_1 | Rnf112 | 3.86E−54 |
| PIMN_1 | Thsd7b | 2.08E−53 |
| PIMN_1 | Cacna1c | 1.62E−52 |
| PIMN_1 | Ptprz1 | 2.77E−52 |
| PIMN_1 | Kcnh6 | 1.07E−51 |
| PIMN_1 | Slc35d3 | 4.59E−51 |
| PIMN_1 | Kcnq4 | 1.29E−49 |
| PIMN_1 | Synpo2 | 3.22E−49 |
| PIMN_1 | Sspo | 2.16E−48 |
| PIMN_1 | Stard13 | 7.17E−48 |
| PIMN_1 | Dach1 | 1.04E−46 |
| PIMN_1 | Kcnj5 | 1.91E−46 |
| PIMN_1 | Sntb1 | 2.51E−46 |
| PIMN_1 | Ntrk3 | 2.65E−46 |
| PIMN_1 | Bves | 1.48E−45 |
| PIMN_1 | Slc44a5 | 1.62E−45 |
| PIMN_1 | Kcnh8 | 4.03E−45 |
| PIMN_1 | Arhgap42 | 6.52E−45 |
| PIMN_1 | Atp2b1 | 7.25E−45 |
| PIMN_1 | Lrrk1 | 7.81E−45 |
| PIMN_1 | Kcnq3 | 1.57E−44 |
| PIMN_1 | Kcnip4 | 3.24E−44 |
| PIMN_1 | Ablim2 | 6.46E−44 |
| PIMN_1 | Kcnj3 | 1.48E−43 |
| PIMN_1 | Ncald | 1.89E−43 |
| PIMN_1 | Ppap2b | 1.98E−43 |
| PIMN_1 | 4930486F22Rik | 6.37E−43 |
| PIMN_1 | Clvs1 | 9.46E−43 |
| PIMN_1 | Srcin1 | 1.77E−42 |
| PIMN_1 | Chd7 | 6.37E−42 |
| PIMN_1 | Plvap | 8.64E−42 |
| PIMN_1 | Unc13c | 1.02E−41 |
| PIMN_1 | Ass1 | 1.52E−41 |
| PIMN_1 | Dgkg | 5.30E−41 |
| PIMN_1 | Slc4a4 | 7.73E−41 |
| PIMN_1 | Rnf182 | 1.39E−40 |
| PIMN_1 | Sipa1l1 | 4.39E−40 |
| PIMN_1 | Dmd | 6.28E−40 |
| PIMN_1 | Rasa4 | 3.29E−39 |
| PIMN_1 | Tmod1 | 4.42E−39 |
| PIMN_1 | Cped1 | 5.39E−39 |
| PIMN_1 | Grid2 | 6.67E−39 |
| PIMN_1 | Il1rapl1 | 7.70E−39 |
| PIMN_1 | Plekha5 | 9.88E−39 |
| PIMN_1 | Akap13 | 1.24E−38 |
| PIMN_1 | Il20ra | 2.60E−38 |
| PIMN_1 | Mfsd4 | 4.97E−38 |
| PIMN_1 | Fstl5 | 4.97E−38 |
| PIMN_1 | Sipa1l2 | 3.16E−37 |
| PIMN_1 | Arhgef10l | 4.30E−37 |
| PIMN_1 | Samd5 | 4.67E−37 |
| PIMN_1 | A130077B15Rik | 4.83E−37 |
| PIMN_1 | Creb5 | 5.57E−37 |
| PIMN_1 | Cox6c | 6.48E−36 |
| PIMN_1 | Man1a | 7.39E−36 |
| PIMN_1 | Padi2 | 5.03E−32 |
| PIMN_1 | Eln | 1.20E−22 |
| PIMN_1 | Sept9 | 8.46E−21 |
| PIMN_1 | Chst9 | 1.85E−16 |
| PIMN_1 | Caskin2 | 3.62E−16 |
| PIMN_1 | Aim1l | 4.13E−16 |
| PIMN_1 | Pcolce | 1.98E−15 |
| PIMN_1 | Fndc1 | 3.64E−15 |
| PIMN_1 | Slco2a1 | 3.42E−12 |
| PIMN_1 | Grpr | 5.17E−12 |
| PIMN_1 | Nnmt | 7.65E−11 |
| PIMN_1 | Pon3 | 9.70E−11 |
| PIMN_1 | Fgfr4 | 3.22E−09 |
| PIMN_1 | Wisp2 | 2.57E−08 |
| PIMN_1 | Gpc4 | 7.49E−08 |
| PIMN_1 | Lsp1 | 1.31E−07 |
| PIMN_1 | Krt24 | 8.05E−07 |
| PIMN_1 | Edaradd | 9.51E−07 |
| PIMN_1 | Gpr12 | 4.92E−06 |
| PIMN_1 | Rdh7 | 5.70E−06 |
| PIMN_1 | Rbm24 | 6.99E−06 |
| PIMN_1 | Lgi2 | 1.99E−05 |
| PIMN_1 | Kdr | 3.00E−05 |
| PIMN_1 | Gm13031 | 3.33E−05 |
| PIMN_1 | Defb33 | 3.90E−05 |
| PIMN_1 | Olah | 8.95E−05 |
| PIMN_1 | Klra10 | 1.63E−04 |
| PIMN_1 | Gldc | 1.72E−04 |
| PIMN_1 | Ces4a | 1.88E−04 |
| PIMN_1 | Crh | 1.96E−04 |
| PIMN_1 | Tnxb | 4.71E−04 |
| PIMN_1 | Prl3b1 | 5.93E−04 |
| PIMN_1 | 4933433H22Rik | 6.27E−04 |
| PIMN_1 | Rdh19 | 6.28E−04 |
| PIMN_1 | Gm20751 | 1.05E−03 |
| PIMN_1 | Msl3l2 | 1.07E−03 |
| PIMN_1 | Dear1 | 1.58E−03 |
| PIMN_1 | Serpinb9b | 1.77E−03 |
| PIMN_1 | Chrna1 | 1.84E−03 |
| PIMN_1 | Syt8 | 1.85E−03 |
| PIMN_1 | Gm6583 | 1.89E−03 |
| PIMN_1 | Cyp1b1 | 2.06E−03 |
| PIMN_1 | Dpys | 2.71E−03 |
| PIMN_1 | Gm2042 | 2.88E−03 |
| PIMN_1 | Dntt | 3.00E−03 |
| PIMN_1 | B930092H01Rik | 3.71E−03 |
| PIMN_1 | Pglyrp2 | 3.88E−03 |
| PIMN_1 | Gimap5 | 4.06E−03 |
| PIMN_1 | Krt40 | 4.31E−03 |
| PIMN_1 | Gpr37 | 5.16E−03 |
| PIMN_1 | 4930548H24Rik | 5.36E−03 |
| PIMN_1 | Cmtm2b | 5.43E−03 |
| PIMN_1 | Hgd | 5.96E−03 |
| PIMN_1 | Cd207 | 6.54E−03 |
| PIMN_1 | Cryaa | 6.67E−03 |
| PIMN_1 | Alpl | 9.03E−03 |
| PIMN_1 | Cfd | 9.66E−03 |
| PIMN_1 | Vmn2r45 | 1.05E−02 |
| PIMN_1 | Krt26 | 1.08E−02 |
| PIMN_1 | 4930467K11Rik | 1.12E−02 |
| PIMN_1 | Mfsd7a | 1.12E−02 |
| PIMN_1 | Olfr119 | 1.19E−02 |
| PIMN_1 | Cd52 | 1.22E−02 |
| PIMN_1 | Gm21276 | 1.27E−02 |
| PIMN_1 | Gm13102 | 1.52E−02 |
| PIMN_1 | Car6 | 1.62E−02 |
| PIMN_1 | Fam26d | 1.68E−02 |
| PIMN_1 | C730027H18Rik | 1.93E−02 |
| PIMN_1 | Duxbl1 | 1.93E−02 |
| PIMN_1 | Tmem89 | 2.07E−02 |
| PIMN_1 | Samt2 | 2.20E−02 |
| PIMN_1 | 4930509K18Rik | 2.25E−02 |
| PIMN_1 | Olfr1030 | 2.38E−02 |
| PIMN_1 | Snora35 | 2.49E−02 |
| PIMN_1 | Fam71f1 | 2.86E−02 |
| PIMN_1 | Xlr | 3.02E−02 |
| PIMN_1 | Asb10 | 3.24E−02 |
| PIMN_1 | Myh7 | 3.31E−02 |
| PIMN_1 | Psg17 | 3.35E−02 |
| PIMN_1 | Gm19424 | 3.47E−02 |
| PIMN_1 | 9530053A07Rik | 3.51E−02 |
| PIMN_1 | l730028E13Rik | 3.58E−02 |
| PIMN_1 | Spdyb | 3.83E−02 |
| PIMN_1 | 1700054M17Rik | 4.19E−02 |
| PIMN_1 | Pabpc2 | 4.31E−02 |
| PIMN_1 | E130018N17Rik | 4.41E−02 |
| PIMN_1 | Lyg2 | 4.76E−02 |
| PIMN_1 | Xlr5b | 4.88E−02 |
| PIMN_2 | Cmah | 2.78E−43 |
| PIMN_2 | Col25a1 | 1.24E−32 |
| PIMN_2 | Pear1 | 1.12E−31 |
| PIMN_2 | Lhfp | 1.12E−31 |
| PIMN_2 | Pde1a | 1.31E−27 |
| PIMN_2 | Ano4 | 1.63E−27 |
| PIMN_2 | Rgs7 | 1.70E−27 |
| PIMN_2 | Dagla | 9.50E−26 |
| PIMN_2 | Asic2 | 9.50E−26 |
| PIMN_2 | 5530401A14Rik | 1.45E−25 |
| PIMN_2 | Krt23 | 3.50E−25 |
| PIMN_2 | Ltk | 3.51E−24 |
| PIMN_2 | Chst15 | 3.85E−24 |
| PIMN_2 | Mfsd4 | 2.17E−23 |
| PIMN_2 | Egfem1 | 5.57E−23 |
| PIMN_2 | Sgcd | 1.14E−22 |
| PIMN_2 | Prkg2 | 1.29E−22 |
| PIMN_2 | Slc1a5 | 1.99E−22 |
| PIMN_2 | Cadps2 | 2.20E−22 |
| PIMN_2 | Zfp536 | 3.31E−22 |
| PIMN_2 | Plch2 | 4.08E−22 |
| PIMN_2 | Kcnh1 | 4.08E−22 |
| PIMN_2 | Cdk18 | 1.05E−21 |
| PIMN_2 | Nos1 | 1.33E−21 |
| PIMN_2 | Ngf | 6.23E−20 |
| PIMN_2 | Ablim2 | 9.76E−20 |
| PIMN_2 | Itga8 | 1.41E−18 |
| PIMN_2 | Ryr2 | 1.43E−18 |
| PIMN_2 | Cyp2s1 | 1.53E−18 |
| PIMN_2 | Rarb | 1.82E−18 |
| PIMN_2 | Asic4 | 2.02E−18 |
| PIMN_2 | Gm21949 | 2.55E−18 |
| PIMN_2 | Rnf144b | 2.55E−18 |
| PIMN_2 | Slc35d3 | 3.02E−18 |
| PIMN_2 | Kirrel3 | 3.39E−18 |
| PIMN_2 | Gpc5 | 6.91E−18 |
| PIMN_2 | Gsg1l | 6.96E−18 |
| PIMN_2 | Gfra1 | 1.24E−17 |
| PIMN_2 | Fat3 | 2.28E−17 |
| PIMN_2 | Asl | 2.79E−17 |
| PIMN_2 | Pde1c | 4.55E−17 |
| PIMN_2 | Creb5 | 6.87E−17 |
| PIMN_2 | Slc4a4 | 1.13E−16 |
| PIMN_2 | Syt7 | 1.80E−16 |
| PIMN_2 | Asap2 | 5.10E−16 |
| PIMN_2 | Ass1 | 7.93E−16 |
| PIMN_2 | Plcb1 | 1.34E−15 |
| PIMN_2 | Lrrc32 | 5.51E−15 |
| PIMN_2 | Lamb1 | 5.58E−15 |
| PIMN_2 | Prkd1 | 6.82E−15 |
| PIMN_2 | Plxnb1 | 9.39E−15 |
| PIMN_2 | Il20ra | 9.46E−15 |
| PIMN_2 | Trpc4 | 1.00E−14 |
| PIMN_2 | Ebf1 | 1.24E−14 |
| PIMN_2 | Kcnj5 | 2.56E−14 |
| PIMN_2 | A730090N16Rik | 4.13E−14 |
| PIMN_2 | Cpne7 | 5.26E−14 |
| PIMN_2 | Mical2 | 6.26E−14 |
| PIMN_2 | Utrn | 6.85E−14 |
| PIMN_2 | Cacna1c | 7.68E−14 |
| PIMN_2 | Schip1 | 7.68E−14 |
| PIMN_2 | Vmn1r41 | 8.42E−14 |
| PIMN_2 | Rgs6 | 1.19E−13 |
| PIMN_2 | P2rx3 | 1.61E−13 |
| PIMN_2 | Kcnd3 | 1.86E−13 |
| PIMN_2 | Postn | 2.13E−13 |
| PIMN_2 | Rab37 | 2.41E−13 |
| PIMN_2 | Map7 | 3.01E−13 |
| PIMN_2 | Tenm2 | 3.11E−13 |
| PIMN_2 | Plekha5 | 3.11E−13 |
| PIMN_2 | Kcnab1 | 8.28E−13 |
| PIMN_2 | Specc1 | 8.98E−13 |
| PIMN_2 | Iqgap2 | 1.36E−12 |
| PIMN_2 | Cers6 | 1.38E−12 |
| PIMN_2 | Sulf1 | 1.52E−12 |
| PIMN_2 | Ldb2 | 1.74E−12 |
| PIMN_2 | Syne1 | 1.77E−12 |
| PIMN_2 | Nxn | 1.79E−12 |
| PIMN_2 | Evpl | 1.97E−12 |
| PIMN_2 | Scml4 | 2.52E−12 |
| PIMN_2 | Etv1 | 2.92E−12 |
| PIMN_2 | Sybu | 3.58E−12 |
| PIMN_2 | Gnb3 | 3.59E−12 |
| PIMN_2 | Sorcs2 | 3.79E−12 |
| PIMN_2 | F2rl2 | 4.63E−12 |
| PIMN_2 | Trpm3 | 5.00E−12 |
| PIMN_2 | Tnr | 5.58E−12 |
| PIMN_2 | Entpd3 | 5.58E−12 |
| PIMN_2 | Vwf | 7.19E−12 |
| PIMN_2 | Tyro3 | 7.80E−12 |
| PIMN_2 | Dppa3 | 8.06E−12 |
| PIMN_2 | Acacb | 1.24E−11 |
| PIMN_2 | Rims3 | 1.36E−11 |
| PIMN_2 | Fbxw14 | 1.39E−11 |
| PIMN_2 | Tmc3 | 1.70E−11 |
| PIMN_2 | Grik3 | 2.03E−11 |
| PIMN_2 | Mlxip | 2.60E−11 |
| PIMN_2 | Csgalnact1 | 3.16E−11 |
| PIMN_2 | Sh3pxd2a | 3.57E−11 |
| PIMN_2 | Fscn3 | 4.21E−11 |
| PIMN_2 | Clec3b | 4.57E−11 |
| PIMN_2 | Slc38a4 | 1.92E−10 |
| PIMN_2 | Htr1d | 9.33E−09 |
| PIMN_2 | Pax2 | 1.16E−08 |
| PIMN_2 | Calr4 | 2.50E−08 |
| PIMN_2 | Lgr6 | 3.79E−08 |
| PIMN_2 | Il9r | 1.10E−07 |
| PIMN_2 | Trim50 | 1.94E−07 |
| PIMN_2 | Otop3 | 2.38E−07 |
| PIMN_2 | Wdr86 | 3.82E−07 |
| PIMN_2 | Afp | 6.58E−07 |
| PIMN_2 | Wnt7a | 1.03E−06 |
| PIMN_2 | Cd4 | 1.12E−06 |
| PIMN_2 | Optc | 1.83E−06 |
| PIMN_2 | Akr1c18 | 4.84E−06 |
| PIMN_2 | Otop2 | 4.89E−06 |
| PIMN_2 | Serpinb2 | 5.95E−06 |
| PIMN_2 | 4930459L07Rik | 7.75E−06 |
| PIMN_2 | Vcam1 | 9.92E−06 |
| PIMN_2 | 2310039L15Rik | 1.41E−05 |
| PIMN_2 | Pecam1 | 1.55E−05 |
| PIMN_2 | Gm216 | 1.79E−05 |
| PIMN_2 | Gm10584 | 1.84E−05 |
| PIMN_2 | 1700010D01Rik | 3.94E−05 |
| PIMN_2 | 4933402N22Rik | 4.04E−05 |
| PIMN_2 | Ramp3 | 5.30E−05 |
| PIMN_2 | 2610028E06Rik | 5.81E−05 |
| PIMN_2 | Tgm6 | 1.38E−04 |
| PIMN_2 | Spink6 | 1.64E−04 |
| PIMN_2 | Gja8 | 2.09E−04 |
| PIMN_2 | Fsd2 | 2.66E−04 |
| PIMN_2 | Arhgap9 | 2.70E−04 |
| PIMN_2 | Trim71 | 3.36E−04 |
| PIMN_2 | 4931429L15Rik | 3.36E−04 |
| PIMN_2 | Rbbp8nl | 5.42E−04 |
| PIMN_2 | Usp44 | 5.45E−04 |
| PIMN_2 | Opalin | 6.98E−04 |
| PIMN_2 | Mmp27 | 7.06E−04 |
| PIMN_2 | 1700066B17Rik | 7.46E−04 |
| PIMN_2 | 4930558J18Rik | 1.32E−03 |
| PIMN_2 | Ttll2 | 1.70E−03 |
| PIMN_2 | Hspg2 | 2.21E−03 |
| PIMN_2 | Gm14461 | 2.70E−03 |
| PIMN_2 | 1700120K04Rik | 2.71E−03 |
| PIMN_2 | Ly9 | 2.82E−03 |
| PIMN_2 | 5430416O09Rik | 3.32E−03 |
| PIMN_2 | H60b | 3.51E−03 |
| PIMN_2 | Rbp3 | 3.83E−03 |
| PIMN_2 | Gm11166 | 3.98E−03 |
| PIMN_2 | Apol11a | 4.03E−03 |
| PIMN_2 | 4933425L06Rik | 4.42E−03 |
| PIMN_2 | Fbxo43 | 5.59E−03 |
| PIMN_2 | Hlx | 5.85E−03 |
| PIMN_2 | Pvrl4 | 6.42E−03 |
| PIMN_2 | Ripk4 | 7.02E−03 |
| PIMN_2 | Necab3 | 7.77E−03 |
| PIMN_2 | Lrrc43 | 8.74E−03 |
| PIMN_2 | Gm10494 | 9.24E−03 |
| PIMN_2 | Apol11b | 1.23E−02 |
| PIMN_2 | Gm9992 | 1.31E−02 |
| PIMN_2 | E230025N22Rik | 1.44E−02 |
| PIMN_2 | Uts2d | 1.46E−02 |
| PIMN_2 | 0610039K10Rik | 1.50E−02 |
| PIMN_2 | Gm10745 | 1.50E−02 |
| PIMN_2 | Krt80 | 1.67E−02 |
| PIMN_2 | Gli2 | 2.03E−02 |
| PIMN_2 | H19 | 2.10E−02 |
| PIMN_2 | C1rl | 2.58E−02 |
| PIMN_2 | Dok2 | 2.60E−02 |
| PIMN_2 | Trpv1 | 2.79E−02 |
| PIMN_2 | Sall4 | 2.87E−02 |
| PIMN_2 | Smok2a | 2.95E−02 |
| PIMN_2 | 4930448F12Rik | 3.16E−02 |
| PIMN_2 | 9630013A20Rik | 3.19E−02 |
| PIMN_2 | Ltb | 3.26E−02 |
| PIMN_2 | Lrat | 3.30E−02 |
| PIMN_2 | Epor | 3.45E−02 |
| PIMN_2 | Wnt4 | 3.59E−02 |
| PIMN_2 | Akr1b7 | 3.84E−02 |
| PIMN_2 | 4732456N10Rik | 4.20E−02 |
| PIMN_2 | Hic1 | 4.89E−02 |
| PIMN_3 | Thsd7b | 1.09E−123 |
| PIMN_3 | Opcml | 2.14E−102 |
| PIMN_3 | Cdh12 | 4.36E−100 |
| PIMN_3 | Rgs6 | 2.62E−93 |
| PIMN_3 | Epha8 | 5.07E−88 |
| PIMN_3 | Thsd7a | 6.67E−88 |
| PIMN_3 | Nos1 | 6.21E−86 |
| PIMN_3 | Vcan | 9.44E−86 |
| PIMN_3 | Hmcn1 | 2.05E−83 |
| PIMN_3 | Dgkb | 3.29E−80 |
| PIMN_3 | Kcnab1 | 1.71E−79 |
| PIMN_3 | Ntrk3 | 1.33E−78 |
| PIMN_3 | Susd4 | 7.76E−77 |
| PIMN_3 | Man1a | 1.62E−76 |
| PIMN_3 | Gria3 | 8.49E−75 |
| PIMN_3 | Tenm3 | 1.55E−74 |
| PIMN_3 | Slc44a5 | 2.26E−71 |
| PIMN_3 | Bves | 6.32E−71 |
| PIMN_3 | Gm2516 | 5.67E−70 |
| PIMN_3 | Hdac9 | 9.05E−70 |
| PIMN_3 | Mfsd4 | 1.84E−68 |
| PIMN_3 | Bglap | 7.72E−66 |
| PIMN_3 | Kcnt2 | 8.27E−66 |
| PIMN_3 | Cadps2 | 4.74E−65 |
| PIMN_3 | Etv1 | 2.19E−63 |
| PIMN_3 | Slc35d3 | 2.94E−63 |
| PIMN_3 | Gfra1 | 2.94E−63 |
| PIMN_3 | Dnahc11 | 2.11E−60 |
| PIMN_3 | Aim | 2.45E−60 |
| PIMN_3 | Fat1 | 2.81E−60 |
| PIMN_3 | Dok5 | 6.73E−59 |
| PIMN_3 | Chrna7 | 3.07E−58 |
| PIMN_3 | Unc13c | 1.26E−57 |
| PIMN_3 | Lgr5 | 3.14E−57 |
| PIMN_3 | Alcam | 1.86E−55 |
| PIMN_3 | Oprd1 | 1.32E−54 |
| PIMN_3 | Auts2 | 1.67E−54 |
| PIMN_3 | Ank2 | 1.73E−54 |
| PIMN_3 | Kcnj3 | 2.25E−54 |
| PIMN_3 | Cacnb2 | 1.22E−50 |
| PIMN_3 | Arid5b | 2.25E−49 |
| PIMN_3 | Stard13 | 5.98E−49 |
| PIMN_3 | Rbfox3 | 2.92E−48 |
| PIMN_3 | Ppfia2 | 4.85E−48 |
| PIMN_3 | Vwa5b1 | 9.31E−48 |
| PIMN_3 | Plekha5 | 3.46E−47 |
| PIMN_3 | Epha5 | 4.50E−47 |
| PIMN_3 | Frmd4a | 1.18E−46 |
| PIMN_3 | Epha6 | 5.66E−46 |
| PIMN_3 | Dpysl3 | 6.50E−46 |
| PIMN_3 | Ppap2b | 7.12E−46 |
| PIMN_3 | Dec | 2.38E−45 |
| PIMN_3 | Arhgap15 | 3.50E−45 |
| PIMN_3 | Fgf14 | 5.63E−45 |
| PIMN_3 | Celf4 | 1.25E−44 |
| PIMN_3 | Wwc2 | 5.76E−44 |
| PIMN_3 | Enpp1 | 1.08E−43 |
| PIMN_3 | Kcnj5 | 2.71E−43 |
| PIMN_3 | Rarb | 7.49E−43 |
| PIMN_3 | Bmp2k | 1.24E−42 |
| PIMN_3 | Il20ra | 1.45E−42 |
| PIMN_3 | Plch2 | 3.65E−42 |
| PIMN_3 | Fam155a | 4.17E−42 |
| PIMN_3 | Syt2 | 5.48E−42 |
| PIMN_3 | Lpp | 7.98E−42 |
| PIMN_3 | Igf2r | 1.75E−41 |
| PIMN_3 | Slit3 | 2.21E−41 |
| PIMN_3 | Igsf21 | 3.27E−41 |
| PIMN_3 | Col5a2 | 2.45E−40 |
| PIMN_3 | A730090N16Rik | 9.16E−40 |
| PIMN_3 | Tmem108 | 1.30E−39 |
| PIMN_3 | Ablim2 | 1.38E−39 |
| PIMN_3 | Pcdhl5 | 3.25E−39 |
| PIMN_3 | Robo1 | 4.41E−39 |
| PIMN_3 | Wipi1 | 5.21E−39 |
| PIMN_3 | Cped1 | 6.87E−39 |
| PIMN_3 | Atp8a2 | 9.72E−39 |
| PIMN_3 | Abca1 | 1.04E−38 |
| PIMN_3 | Tcf7l1 | 2.69E−38 |
| PIMN_3 | Dusp15 | 3.37E−38 |
| PIMN_3 | Creb5 | 6.58E−38 |
| PIMN_3 | Gm5607 | 1.04E−37 |
| PIMN_3 | Pdlim5 | 2.42E−37 |
| PIMN_3 | Slc35f1 | 2.82E−37 |
| PIMN_3 | Gm11602 | 1.56E−36 |
| PIMN_3 | Sntb1 | 1.67E−36 |
| PIMN_3 | P2rx2 | 1.77E−36 |
| PIMN_3 | Clvs1 | 2.12E−36 |
| PIMN_3 | Pcdh9 | 2.91E−36 |
| PIMN_3 | Gm14391 | 4.53E−36 |
| PIMN_3 | Grb14 | 8.10E−36 |
| PIMN_3 | Synpo2 | 8.10E−36 |
| PIMN_3 | Tnr | 2.83E−35 |
| PIMN_3 | Plekha7 | 3.86E−35 |
| PIMN_3 | Adamts5 | 1.51E−34 |
| PIMN_3 | Lama5 | 6.22E−34 |
| PIMN_3 | Cacna1d | 1.51E−33 |
| PIMN_3 | Kcnq4 | 1.66E−33 |
| PIMN_3 | Gnal | 1.77E−33 |
| PIMN_3 | Ccnjl | 3.51E−33 |
| PIMN_3 | Bglap2 | 4.19E−33 |
| PIMN_3 | Mpz | 8.28E−28 |
| PIMN_3 | Exph5 | 1.20E−25 |
| PIMN_3 | Ptch2 | 1.96E−25 |
| PIMN_3 | Mmd2 | 3.21E−24 |
| PIMN_3 | Grin2b | 1.48E−22 |
| PIMN_3 | Cox8b | 2.21E−20 |
| PIMN_3 | Apcdd1 | 4.83E−20 |
| PIMN_3 | 3110039I08Rik | 1.69E−18 |
| PIMN_3 | Gfpt2 | 9.69E−17 |
| PIMN_3 | Myo10 | 9.27E−16 |
| PIMN_3 | Rhbdf2 | 4.18E−15 |
| PIMN_3 | Ar | 2.41E−11 |
| PIMN_3 | Sox8 | 9.05E−11 |
| PIMN_3 | Sox2ot | 4.39E−09 |
| PIMN_3 | Npy | 1.18E−08 |
| PIMN_3 | Eda2r | 1.90E−06 |
| PIMN_3 | Gpr88 | 4.76E−06 |
| PIMN_3 | Klk1b1 | 1.04E−05 |
| PIMN_3 | Spn-ps | 1.37E−05 |
| PIMN_3 | Runx3 | 1.88E−05 |
| PIMN_3 | Pipox | 2.15E−05 |
| PIMN_3 | 2310030G06Rik | 4.03E−05 |
| PIMN_3 | Lmo2 | 5.40E−05 |
| PIMN_3 | Serpina3c | 7.46E−05 |
| PIMN_3 | Fzd10 | 1.06E−04 |
| PIMN_3 | A930009A15Rik | 1.09E−04 |
| PIMN_3 | 1700085C21Rik | 1.15E−04 |
| PIMN_3 | Ajap1 | 1.29E−04 |
| PIMN_3 | H1foo | 1.63E−04 |
| PIMN_3 | Gm15091 | 1.71E−04 |
| PIMN_3 | Smyd1 | 1.72E−04 |
| PIMN_3 | Meox2 | 2.22E−04 |
| PIMN_3 | Gm20743 | 3.11E−04 |
| PIMN_3 | Ripk3 | 3.56E−04 |
| PIMN_3 | Foxo6 | 4.18E−04 |
| PIMN_3 | Serpina3i | 4.88E−04 |
| PIMN_3 | Nr2f1 | 6.44E−04 |
| PIMN_3 | Ifi44l | 9.46E−04 |
| PIMN_3 | Serpina3b | 1.16E−03 |
| PIMN_3 | 4930548J01Rik | 1.36E−03 |
| PIMN_3 | 2900002K06Rik | 1.44E−03 |
| PIMN_3 | Xcl1 | 1.70E−03 |
| PIMN_3 | Cdca5 | 1.90E−03 |
| PIMN_3 | Slc25a48 | 2.92E−03 |
| PIMN_3 | Vmn2r69 | 2.94E−03 |
| PIMN_3 | 2700046A07Rik | 3.55E−03 |
| PIMN_3 | Htr5a | 3.60E−03 |
| PIMN_3 | 1700049E22Rik | 3.65E−03 |
| PIMN_3 | Fam47e | 3.65E−03 |
| PIMN_3 | Nlrp4f | 3.66E−03 |
| PIMN_3 | 2310034O05Rik | 3.73E−03 |
| PIMN_3 | Cd8a | 3.96E−03 |
| PIMN_3 | Foxs1 | 4.87E−03 |
| PIMN_3 | Prox1 | 5.02E−03 |
| PIMN_3 | Tuba8 | 7.51E−03 |
| PIMN_3 | Aadacl3 | 9.29E−03 |
| PIMN_3 | Lox | 9.56E−03 |
| PIMN_3 | Lyg1 | 9.61E−03 |
| PIMN_3 | Ctf2 | 1.38E−02 |
| PIMN_3 | Gm5549 | 1.65E−02 |
| PIMN_3 | Qrfp | 1.73E−02 |
| PIMN_3 | 4930433I11Rik | 1.75E−02 |
| PIMN_3 | 1190002F15Rik | 1.80E−02 |
| PIMN_3 | Gm7168 | 2.16E−02 |
| PIMN_3 | Tmem52 | 2.19E−02 |
| PIMN_3 | Kcnj4 | 2.22E−02 |
| PIMN_3 | Treml2 | 2.26E−02 |
| PIMN_3 | Gm4858 | 2.30E−02 |
| PIMN_3 | Pgc | 2.49E−02 |
| PIMN_3 | Fabp6 | 2.70E−02 |
| PIMN_3 | Cdc20 | 2.95E−02 |
| PIMN_3 | 9230110F15Rik | 2.99E−02 |
| PIMN_3 | Gm11756 | 2.99E−02 |
| PIMN_3 | Hemgn | 3.02E−02 |
| PIMN_3 | Psapl1 | 3.05E−02 |
| PIMN_3 | 9130209A04Rik | 3.13E−02 |
| PIMN_3 | Cd3d | 3.44E−02 |
| PIMN_3 | Spta1 | 3.58E−02 |
| PIMN_3 | Lef1 | 3.63E−02 |
| PIMN_3 | Cyp4a29-ps | 3.65E−02 |
| PIMN_3 | Cdcp2 | 3.70E−02 |
| PIMN_3 | Mtl5 | 3.91E−02 |
| PIMN_3 | 5430440P10Rik | 4.03E−02 |
| PIMN_3 | Ctla4 | 4.15E−02 |
| PIMN_3 | Spin4 | 4.24E−02 |
| PIMN_3 | 7420461P10Rik | 4.46E−02 |
| PIMN_3 | Cga | 4.97E−02 |
| PIMN_4 | Ltbp1 | 4.74E−104 |
| PIMN_4 | Cttnbp2 | 6.26E−96 |
| PIMN_4 | Thsd7a | 1.29E−95 |
| PIMN_4 | Thsd7b | 7.98E−94 |
| PIMN_4 | Vcan | 3.49E−85 |
| PIMN_4 | Col7a1 | 2.48E−78 |
| PIMN_4 | Dec | 6.72E−74 |
| PIMN_4 | Vwa5b1 | 1.37E−71 |
| PIMN_4 | Opcml | 3.88E−71 |
| PIMN_4 | Tenm3 | 3.12E−63 |
| PIMN_4 | Rgs6 | 4.56E−62 |
| PIMN_4 | Nos1 | 9.62E−61 |
| PIMN_4 | Ntrk3 | 3.47E−55 |
| PIMN_4 | Fat1 | 6.35E−53 |
| PIMN_4 | Gfra1 | 3.06E−52 |
| PIMN_4 | Unc13c | 5.70E−52 |
| PIMN_4 | Kcnj3 | 2.09E−50 |
| PIMN_4 | Igf1r | 3.05E−49 |
| PIMN_4 | Gm5607 | 2.48E−48 |
| PIMN_4 | Dok5 | 3.19E−47 |
| PIMN_4 | Etv1 | 2.57E−46 |
| PIMN_4 | Fgf14 | 2.73E−45 |
| PIMN_4 | Airn | 6.25E−44 |
| PIMN_4 | Creb5 | 1.48E−43 |
| PIMN_4 | Cacnb2 | 1.48E−43 |
| PIMN_4 | Ptprg | 6.80E−43 |
| PIMN_4 | Lpp | 9.44E−42 |
| PIMN_4 | Kcnh8 | 1.11E−41 |
| PIMN_4 | Gm2516 | 1.62E−41 |
| PIMN_4 | Bves | 5.24E−41 |
| PIMN_4 | Oprd1 | 6.15E−41 |
| PIMN_4 | Stab2 | 6.04E−40 |
| PIMN_4 | Slc44a5 | 9.32E−40 |
| PIMN_4 | Mboat2 | 4.23E−39 |
| PIMN_4 | Gabrb2 | 5.90E−39 |
| PIMN_4 | Dach1 | 6.05E−39 |
| PIMN_4 | Cacna1d | 1.08E−38 |
| PIMN_4 | Syn3 | 1.30E−38 |
| PIMN_4 | Lama5 | 3.39E−38 |
| PIMN_4 | Epha8 | 5.93E−38 |
| PIMN_4 | Mfsd4 | 8.22E−37 |
| PIMN_4 | Col5a2 | 1.38E−36 |
| PIMN_4 | Kcnj5 | 1.80E−36 |
| PIMN_4 | Ppap2b | 1.87E−36 |
| PIMN_4 | Timp3 | 2.18E−36 |
| PIMN_4 | Asap1 | 4.28E−36 |
| PIMN_4 | A530058N18Rik | 9.41E−36 |
| PIMN_4 | Kcnab1 | 9.41E−36 |
| PIMN_4 | Man1a | 1.46E−35 |
| PIMN_4 | Slc35d3 | 4.20E−35 |
| PIMN_4 | Kcnt2 | 1.03E−34 |
| PIMN_4 | Tmem108 | 3.11E−34 |
| PIMN_4 | C1ql1 | 1.09E−33 |
| PIMN_4 | Ccnjl | 3.51E−33 |
| PIMN_4 | Rnf144b | 8.68E−33 |
| PIMN_4 | Ablim2 | 1.26E−32 |
| PIMN_4 | Arhgef26 | 5.91E−32 |
| PIMN_4 | Zfp536 | 1.66E−31 |
| PIMN_4 | Gulp1 | 1.85E−31 |
| PIMN_4 | Sntb1 | 2.96E−31 |
| PIMN_4 | Dnahc11 | 1.23E−30 |
| PIMN_4 | Clvs1 | 1.31E−30 |
| PIMN_4 | Rarb | 1.79E−30 |
| PIMN_4 | Cacna1c | 1.94E−30 |
| PIMN_4 | Ank2 | 2.19E−30 |
| PIMN_4 | Fbxo7 | 5.21E−30 |
| PIMN_4 | Tcf7l1 | 9.25E−30 |
| PIMN_4 | 1700113H08Rik | 1.24E−29 |
| PIMN_4 | Sox8 | 2.91E−29 |
| PIMN_4 | Caln1 | 3.46E−29 |
| PIMN_4 | Hmcn1 | 4.91E−29 |
| PIMN_4 | Entpd3 | 3.55E−28 |
| PIMN_4 | Igf2r | 6.27E−28 |
| PIMN_4 | Srcin1 | 9.75E−28 |
| PIMN_4 | Gm14718 | 1.06E−27 |
| PIMN_4 | Sgk1 | 1.21E−27 |
| PIMN_4 | Ncald | 1.36E−27 |
| PIMN_4 | Synpo2 | 3.44E−27 |
| PIMN_4 | Alcam | 4.22E−27 |
| PIMN_4 | Kcnq4 | 5.56E−27 |
| PIMN_4 | Wipi1 | 7.51E−27 |
| PIMN_4 | Ptchd1 | 1.16E−26 |
| PIMN_4 | Auts2 | 1.84E−26 |
| PIMN_4 | Bmper | 5.05E−26 |
| PIMN_4 | Cped1 | 5.58E−26 |
| PIMN_4 | Spsb4 | 8.03E−26 |
| PIMN_4 | Wwc2 | 4.02E−25 |
| PIMN_4 | Epha5 | 4.11E−25 |
| PIMN_4 | Afap1l1 | 5.18E−25 |
| PIMN_4 | Acpl2 | 6.32E−25 |
| PIMN_4 | Ass1 | 7.60E−25 |
| PIMN_4 | Dock9 | 1.67E−24 |
| PIMN_4 | Frmd4a | 2.04E−24 |
| PIMN_4 | Sema3a | 2.11E−24 |
| PIMN_4 | Popdc3 | 8.71E−24 |
| PIMN_4 | Robo1 | 2.96E−23 |
| PIMN_4 | Pde1c | 5.46E−23 |
| PIMN_4 | Ppm1h | 6.79E−23 |
| PIMN_4 | Oprm1 | 7.65E−23 |
| PIMN_4 | Fam155a | 9.85E−23 |
| PIMN_4 | Efcc1 | 1.13E−22 |
| PIMN_4 | Nptx1 | 1.63E−20 |
| PIMN_4 | 5930412G12Rik | 4.61E−19 |
| PIMN_4 | Igdcc3 | 1.85E−17 |
| PIMN_4 | Sox2ot | 6.40E−14 |
| PIMN_4 | Lmx1b | 9.93E−13 |
| PIMN_4 | Lipf | 6.31E−09 |
| PIMN_4 | Ptgds | 2.28E−07 |
| PIMN_4 | E030019B06Rik | 3.64E−07 |
| PIMN_4 | Ucn2 | 4.05E−07 |
| PIMN_4 | 1700007F19Rik | 8.60E−07 |
| PIMN_4 | Lpin3 | 1.72E−06 |
| PIMN_4 | Tm4sf5 | 8.11E−06 |
| PIMN_4 | Stfa1 | 8.53E−06 |
| PIMN_4 | Sox2 | 1.07E−05 |
| PIMN_4 | Gm10046 | 1.60E−05 |
| PIMN_4 | Fgf5 | 1.68E−05 |
| PIMN_4 | Magix | 4.75E−05 |
| PIMN_4 | 4930413E15Rik | 6.73E−05 |
| PIMN_4 | Slc25a34 | 8.15E−05 |
| PIMN_4 | Nobox | 8.61E−05 |
| PIMN_4 | Ldhal6b | 1.11E−04 |
| PIMN_4 | Klk1b4 | 1.54E−04 |
| PIMN_4 | Slitrk6 | 1.67E−04 |
| PIMN_4 | Lta | 1.74E−04 |
| PIMN_4 | 4930545E07Rik | 1.85E−04 |
| PIMN_4 | Klhl31 | 2.17E−04 |
| PIMN_4 | 4933432G23Rik | 2.73E−04 |
| PIMN_4 | Trim10 | 3.88E−04 |
| PIMN_4 | Gm3286 | 5.36E−04 |
| PIMN_4 | Krt28 | 5.95E−04 |
| PIMN_4 | Slc13a3 | 6.06E−04 |
| PIMN_4 | Hist2h3c1 | 6.47E−04 |
| PIMN_4 | Lmod3 | 9.68E−04 |
| PIMN_4 | Mir100 | 1.10E−03 |
| PIMN_4 | Lctl | 1.44E−03 |
| PIMN_4 | Olfr970 | 2.40E−03 |
| PIMN_4 | Il3 | 3.40E−03 |
| PIMN_4 | Set | 4.29E−03 |
| PIMN_4 | Nid2 | 4.54E−03 |
| PIMN_4 | Vmn2r66 | 4.83E−03 |
| PIMN_4 | Defb7 | 5.85E−03 |
| PIMN_4 | Tyrp1 | 6.08E−03 |
| PIMN_4 | 1700051A21Rik | 6.09E−03 |
| PIMN_4 | 4930425O10Rik | 7.44E−03 |
| PIMN_4 | Gprc5c | 9.28E−03 |
| PIMN_4 | Prss38 | 1.20E−02 |
| PIMN_4 | Tbx22 | 1.21E−02 |
| PIMN_4 | 1700012B09Rik | 1.25E−02 |
| PIMN_4 | Wfdc8 | 1.30E−02 |
| PIMN_4 | Gm10790 | 1.42E−02 |
| PIMN_4 | Trim42 | 1.57E−02 |
| PIMN_4 | Sash3 | 1.59E−02 |
| PIMN_4 | Wisp1 | 1.75E−02 |
| PIMN_4 | AU022751 | 1.76E−02 |
| PIMN_4 | Mug1 | 1.81E−02 |
| PIMN_4 | Prl2a1 | 1.85E−02 |
| PIMN_4 | Trim69 | 1.87E−02 |
| PIMN_4 | 1700003G13Rik | 1.89E−02 |
| PIMN_4 | Serpinb6d | 2.01E−02 |
| PIMN_4 | Slc7a9 | 2.02E−02 |
| PIMN_4 | Gm16405 | 2.15E−02 |
| PIMN_4 | Defb6 | 2.20E−02 |
| PIMN_4 | Acsm4 | 2.21E−02 |
| PIMN_4 | Gm16430 | 2.29E−02 |
| PIMN_4 | Treml4 | 2.35E−02 |
| PIMN_4 | Prok1 | 2.81E−02 |
| PIMN_4 | Gm5166 | 2.85E−02 |
| PIMN_4 | Pira4 | 2.90E−02 |
| PIMN_4 | Tgtp1 | 2.95E−02 |
| PIMN_4 | Prss51 | 3.00E−02 |
| PIMN_4 | 1700018C11Rik | 3.01E−02 |
| PIMN_4 | Krt27 | 3.02E−02 |
| PIMN_4 | Cdkn2a | 3.03E−02 |
| PIMN_4 | Mir1929 | 3.32E−02 |
| PIMN_4 | Prss22 | 3.45E−02 |
| PIMN_4 | Cpb1 | 3.45E−02 |
| PIMN_4 | Ces1e | 3.47E−02 |
| PIMN_4 | Tcl1b2 | 3.51E−02 |
| PIMN_4 | Wfdc15b | 3.84E−02 |
| PIMN_4 | Xlr4c | 3.93E−02 |
| PIMN_4 | 1700001F09Rik | 3.96E−02 |
| PIMN_4 | Ppp1r17 | 4.04E−02 |
| PIMN_4 | 4930556C24Rik | 4.08E−02 |
| PIMN_4 | Fbp1 | 4.35E−02 |
| PIMN_4 | Scarna13 | 4.36E−02 |
| PIMN_4 | Prl3c1 | 4.53E−02 |
| PIMN_4 | Selp | 4.61E−02 |
| PIMN_4 | Ccl8 | 4.75E−02 |
| PIMN_4 | Trim30b | 4.80E−02 |
| PIMN_4 | 4930572013Rik | 4.87E−02 |
| PIMN_5 | Dgkb | 1.87E−65 |
| PIMN_5 | Cmah | 2.96E−59 |
| PIMN_5 | Rarb | 3.31E−58 |
| PIMN_5 | Hmcn1 | 2.24E−56 |
| PIMN_5 | Sorcs3 | 3.34E−56 |
| PIMN_5 | Epha8 | 2.63E−51 |
| PIMN_5 | Gria3 | 1.11E−46 |
| PIMN_5 | Eya4 | 2.48E−44 |
| PIMN_5 | Dpp10 | 1.82E−43 |
| PIMN_5 | Gsg1l | 4.95E−43 |
| PIMN_5 | Rgs6 | 7.08E−43 |
| PIMN_5 | Stra8 | 1.43E−41 |
| PIMN_5 | Cdh12 | 1.51E−41 |
| PIMN_5 | Dach2 | 8.14E−41 |
| PIMN_5 | Cyct | 2.74E−39 |
| PIMN_5 | Cadps2 | 1.96E−38 |
| PIMN_5 | Plch2 | 5.02E−38 |
| PIMN_5 | Mfsd4 | 7.15E−37 |
| PIMN_5 | Nxn | 5.16E−36 |
| PIMN_5 | Slc35d3 | 5.56E−35 |
| PIMN_5 | Nos1 | 8.41E−35 |
| PIMN_5 | Bves | 2.36E−33 |
| PIMN_5 | Rgs7 | 3.52E−33 |
| PIMN_5 | Gfra1 | 4.06E−33 |
| PIMN_5 | Sorcs2 | 7.11E−32 |
| PIMN_5 | Col25a1 | 9.08E−32 |
| PIMN_5 | Rapgef3 | 1.76E−31 |
| PIMN_5 | Slc39a12 | 2.48E−31 |
| PIMN_5 | Igsf21 | 6.47E−31 |
| PIMN_5 | Alcam | 7.92E−30 |
| PIMN_5 | Grb14 | 3.87E−27 |
| PIMN_5 | Gas6 | 2.49E−26 |
| PIMN_5 | Etv1 | 2.67E−26 |
| PIMN_5 | Tmc3 | 5.23E−26 |
| PIMN_5 | Fat3 | 9.62E−26 |
| PIMN_5 | Creb5 | 2.84E−25 |
| PIMN_5 | Pcdh9 | 4.31E−25 |
| PIMN_5 | Slc6a1 | 5.03E−25 |
| PIMN_5 | Vwa5b1 | 1.09E−24 |
| PIMN_5 | Zfp804a | 2.81E−24 |
| PIMN_5 | Tmem196 | 4.82E−24 |
| PIMN_5 | Grik3 | 6.18E−24 |
| PIMN_5 | Pcdh15 | 6.18E−24 |
| PIMN_5 | Slc4a4 | 1.28E−23 |
| PIMN_5 | Tenm2 | 1.82E−23 |
| PIMN_5 | Rbfox3 | 2.30E−23 |
| PIMN_5 | Ablim2 | 1.20E−22 |
| PIMN_5 | Rnf144b | 1.61E−22 |
| PIMN_5 | Prkd1 | 3.64E−22 |
| PIMN_5 | Il20ra | 1.29E−21 |
| PIMN_5 | Egfem1 | 2.02E−21 |
| PIMN_5 | Plekha7 | 3.18E−21 |
| PIMN_5 | Cacnb2 | 3.97E−21 |
| PIMN_5 | Gpr98 | 7.99E−21 |
| PIMN_5 | Auts2 | 1.41E−20 |
| PIMN_5 | Mkx | 1.70E−20 |
| PIMN_5 | Ltk | 1.98E−20 |
| PIMN_5 | Slc44a5 | 2.14E−20 |
| PIMN_5 | Khdrbs2 | 2.30E−20 |
| PIMN_5 | Ryr2 | 2.53E−20 |
| PIMN_5 | Arhgap15 | 3.48E−20 |
| PIMN_5 | 4930428E07Rik | 5.74E−20 |
| PIMN_5 | Wwc2 | 6.31E−20 |
| PIMN_5 | Syt2 | 7.22E−20 |
| PIMN_5 | Rtn4rl1 | 1.15E−19 |
| PIMN_5 | Stxbp6 | 1.54E−19 |
| PIMN_5 | Dagla | 2.34E−19 |
| PIMN_5 | Plekha5 | 3.17E−19 |
| PIMN_5 | Epha5 | 6.58E−19 |
| PIMN_5 | Cyp2s1 | 7.62E−19 |
| PIMN_5 | Gtsf1l | 1.65E−18 |
| PIMN_5 | Kcnab1 | 2.42E−18 |
| PIMN_5 | Gm11602 | 4.46E−18 |
| PIMN_5 | Tspan18 | 4.64E−18 |
| PIMN_5 | Tmem255b | 6.83E−18 |
| PIMN_5 | Ank2 | 6.88E−18 |
| PIMN_5 | Gpc5 | 9.27E−18 |
| PIMN_5 | Kcnt2 | 2.72E−17 |
| PIMN_5 | Tmem150c | 3.40E−17 |
| PIMN_5 | Dock6 | 4.59E−17 |
| PIMN_5 | Ptch2 | 9.26E−17 |
| PIMN_5 | Kcnq4 | 9.70E−17 |
| PIMN_5 | Dgkg | 9.96E−17 |
| PIMN_5 | Schip1 | 1.17E−16 |
| PIMN_5 | Fbn1 | 1.29E−16 |
| PIMN_5 | P2ry6 | 1.32E−16 |
| PIMN_5 | Cobll1 | 1.33E−16 |
| PIMN_5 | Wipi1 | 2.35E−16 |
| PIMN_5 | A530058N18Rik | 3.67E−16 |
| PIMN_5 | Clmp | 4.08E−16 |
| PIMN_5 | Grem2 | 5.89E−16 |
| PIMN_5 | Arid5b | 5.89E−16 |
| PIMN_5 | Nbas | 5.89E−16 |
| PIMN_5 | Ass1 | 6.34E−16 |
| PIMN_5 | Camk4 | 7.86E−16 |
| PIMN_5 | Bglap | 1.79E−15 |
| PIMN_5 | Gucy1a2 | 2.30E−15 |
| PIMN_5 | C1ql1 | 2.60E−15 |
| PIMN_5 | Kcnq5 | 2.60E−15 |
| PIMN_5 | Ptprz1 | 3.47E−15 |
| PIMN_5 | Kcnk9 | 6.74E−15 |
| PIMN_5 | Rhox4f | 9.70E−13 |
| PIMN_5 | Pbp2 | 1.64E−12 |
| PIMN_5 | Hcrtr1 | 8.31E−12 |
| PIMN_5 | Vmn2r52 | 9.27E−09 |
| PIMN_5 | Btnl6 | 1.05E−06 |
| PIMN_5 | Uox | 1.75E−06 |
| PIMN_5 | Ttll8 | 5.25E−06 |
| PIMN_5 | C130079G13Rik | 7.14E−06 |
| PIMN_5 | Wnt10a | 1.23E−05 |
| PIMN_5 | Igf2bp1 | 1.57E−05 |
| PIMN_5 | Anxa10 | 1.61E−05 |
| PIMN_5 | Obox2 | 4.30E−05 |
| PIMN_5 | Gm14207 | 5.69E−05 |
| PIMN_5 | 2610018G03Rik | 9.63E−05 |
| PIMN_5 | Lrrc32 | 1.06E−04 |
| PIMN_5 | Bcl11a | 1.16E−04 |
| PIMN_5 | Itgad | 1.17E−04 |
| PIMN_5 | Kcnh3 | 1.36E−04 |
| PIMN_5 | Dmrtc1a | 1.50E−04 |
| PIMN_5 | H2-Eb2 | 1.57E−04 |
| PIMN_5 | Fam159a | 2.77E−04 |
| PIMN_5 | Dmp1 | 3.75E−04 |
| PIMN_5 | Ucn2 | 4.34E−04 |
| PIMN_5 | 1700049E15Rik | 4.96E−04 |
| PIMN_5 | 5430416O09Rik | 6.03E−04 |
| PIMN_5 | Arrdc5 | 6.17E−04 |
| PIMN_5 | Macc1 | 7.43E−04 |
| PIMN_5 | Srms | 8.04E−04 |
| PIMN_5 | Cyp2a12 | 8.60E−04 |
| PIMN_5 | Krtap10-10 | 9.70E−04 |
| PIMN_5 | Cd96 | 1.05E−03 |
| PIMN_5 | Scn10a | 1.37E−03 |
| PIMN_5 | 4933400A11Rik | 1.53E−03 |
| PIMN_5 | 8430437L04Rik | 1.57E−03 |
| PIMN_5 | Ndufs5 | 1.80E−03 |
| PIMN_5 | Gm216 | 2.42E−03 |
| PIMN_5 | Asic5 | 2.48E−03 |
| PIMN_5 | Tmem27 | 2.62E−03 |
| PIMN_5 | Zc3h12d | 2.81E−03 |
| PIMN_5 | 4933406K04Rik | 2.89E−03 |
| PIMN_5 | Lrcol1 | 2.91E−03 |
| PIMN_5 | Gm19784 | 3.02E−03 |
| PIMN_5 | Gm16796 | 3.02E−03 |
| PIMN_5 | Fcgr4 | 3.55E−03 |
| PIMN_5 | Gm19434 | 3.81E−03 |
| PIMN_5 | Zbtb12 | 3.82E−03 |
| PIMN_5 | Cxcl5 | 4.17E−03 |
| PIMN_5 | Gm15114 | 4.55E−03 |
| PIMN_5 | Nrl | 5.51E−03 |
| PIMN_5 | 9530002B09Rik | 6.01E−03 |
| PIMN_5 | Luzp4 | 6.42E−03 |
| PIMN_5 | 4930564B18Rik | 6.95E−03 |
| PIMN_5 | 4933402J15Rik | 7.37E−03 |
| PIMN_5 | 4931431B13Rik | 7.37E−03 |
| PIMN_5 | C86187 | 8.55E−03 |
| PIMN_5 | 2410004I01Rik | 8.83E−03 |
| PIMN_5 | Csn1s1 | 9.33E−03 |
| PIMN_5 | Lbp | 9.94E−03 |
| PIMN_5 | Snord4a | 1.13E−02 |
| PIMN_5 | Gpr142 | 1.30E−02 |
| PIMN_5 | Ms4a13 | 1.32E−02 |
| PIMN_5 | Hsh2d | 1.35E−02 |
| PIMN_5 | Fpr1 | 1.61E−02 |
| PIMN_5 | Foxn4 | 1.64E−02 |
| PIMN_5 | Chia | 1.73E−02 |
| PIMN_5 | Scarf2 | 1.84E−02 |
| PIMN_5 | Accsl | 2.15E−02 |
| PIMN_5 | Kcne2 | 2.19E−02 |
| PIMN_5 | 4933425B07Rik | 2.35E−02 |
| PIMN_5 | Lgi3 | 2.67E−02 |
| PIMN_5 | Klk7 | 2.69E−02 |
| PIMN_5 | Was | 2.76E−02 |
| PIMN_5 | Topaz1 | 2.86E−02 |
| PIMN_5 | Gm17751 | 2.88E−02 |
| PIMN_5 | Gm156 | 2.92E−02 |
| PIMN_5 | Mpo | 3.01E−02 |
| PIMN_5 | Fam209 | 3.08E−02 |
| PIMN_5 | 4933422H20Rik | 3.12E−02 |
| PIMN_5 | Gm9920 | 3.24E−02 |
| PIMN_5 | Lrrc52 | 3.48E−02 |
| PIMN_5 | Fam71b | 3.59E−02 |
| PIMN_5 | Il19 | 4.37E−02 |
| PIMN_5 | Tgm5 | 4.47E−02 |
| PIMN_5 | Myh3 | 4.47E−02 |
| PIMN_5 | Cd40lg | 4.75E−02 |
| PIMN_5 | AB099516 | 4.84E−02 |
| PIMN_6 | Chga | 2.33E−43 |
| PIMN_6 | Cygb | 4.97E−42 |
| PIMN_6 | Bglap2 | 4.44E−36 |
| PIMN_6 | Bglap | 4.44E−36 |
| PIMN_6 | C1ql1 | 7.87E−33 |
| PIMN_6 | Dkk3 | 1.16E−32 |
| PIMN_6 | Ctsb | 5.73E−32 |
| PIMN_6 | Rprml | 4.76E−31 |
| PIMN_6 | Cd80 | 4.66E−30 |
| PIMN_6 | Ccdc11 | 5.52E−30 |
| PIMN_6 | Ngb | 2.68E−29 |
| PIMN_6 | Ngfr | 1.71E−28 |
| PIMN_6 | Crabp1 | 7.82E−28 |
| PIMN_6 | Tmem176b | 8.45E−28 |
| PIMN_6 | Gal | 1.85E−27 |
| PIMN_6 | Gas6 | 4.66E−27 |
| PIMN_6 | Vip | 7.12E−27 |
| PIMN_6 | Gsg1l | 2.23E−26 |
| PIMN_6 | Tubb3 | 2.39E−26 |
| PIMN_6 | Qdpr | 4.18E−26 |
| PIMN_6 | S100a16 | 2.09E−25 |
| PIMN_6 | Slc35d3 | 2.64E−25 |
| PIMN_6 | Defb40 | 3.04E−25 |
| PIMN_6 | Mfsd4 | 2.27E−24 |
| PIMN_6 | Slc22a8 | 3.41E−24 |
| PIMN_6 | Ptgir | 9.90E−24 |
| PIMN_6 | Epha8 | 2.54E−23 |
| PIMN_6 | Plch2 | 2.54E−23 |
| PIMN_6 | Dgkb | 4.52E−23 |
| PIMN_6 | S100a6 | 1.27E−22 |
| PIMN_6 | Aldoart1 | 2.32E−22 |
| PIMN_6 | Aldoart2 | 2.86E−22 |
| PIMN_6 | Ppia | 4.20E−22 |
| PIMN_6 | Vmn2r-ps54 | 4.65E−22 |
| PIMN_6 | Ass1 | 4.94E−22 |
| PIMN_6 | Hmcn1 | 5.20E−22 |
| PIMN_6 | Slc6a1 | 6.99E−22 |
| PIMN_6 | Cyp2s1 | 3.56E−21 |
| PIMN_6 | Adcy2 | 4.05E−21 |
| PIMN_6 | Ctsf | 5.11E−21 |
| PIMN_6 | Slc7a11 | 5.71E−21 |
| PIMN_6 | Skint6 | 8.61E−21 |
| PIMN_6 | Skint10 | 2.02E−20 |
| PIMN_6 | Cmah | 2.56E−20 |
| PIMN_6 | Bglap3 | 3.02E−20 |
| PIMN_6 | Kcnab2 | 3.48E−20 |
| PIMN_6 | Abhd3 | 5.36E−20 |
| PIMN_6 | Gm6682 | 5.36E−20 |
| PIMN_6 | Cdh12 | 5.40E−20 |
| PIMN_6 | Ckb | 1.91E−19 |
| PIMN_6 | Gm12070 | 2.18E−19 |
| PIMN_6 | Tuba1a | 4.39E−19 |
| PIMN_6 | Hcrtr1 | 5.23E−19 |
| PIMN_6 | Vat1 | 6.18E−19 |
| PIMN_6 | Cartpt | 7.95E−19 |
| PIMN_6 | Dbh | 9.80E−19 |
| PIMN_6 | Nsg2 | 1.01E−18 |
| PIMN_6 | Bves | 1.21E−18 |
| PIMN_6 | Aldoa | 1.88E−18 |
| PIMN_6 | Eya4 | 2.15E−18 |
| PIMN_6 | Gclm | 2.15E−18 |
| PIMN_6 | Tuba1b | 3.42E−18 |
| PIMN_6 | Tppp3 | 4.71E−18 |
| PIMN_6 | Camp | 9.96E−18 |
| PIMN_6 | Nos1 | 9.96E−18 |
| PIMN_6 | Gria3 | 9.96E−18 |
| PIMN_6 | Sele | 1.61E−17 |
| PIMN_6 | Abhd12b | 2.68E−17 |
| PIMN_6 | Kcng4 | 3.23E−17 |
| PIMN_6 | Il20ra | 3.23E−17 |
| PIMN_6 | Pcsk6 | 3.23E−17 |
| PIMN_6 | Atp6ap2 | 3.95E−17 |
| PIMN_6 | Rgs6 | 4.46E−17 |
| PIMN_6 | Adm | 4.62E−17 |
| PIMN_6 | Phyhip | 6.74E−17 |
| PIMN_6 | Cplx2 | 1.57E−16 |
| PIMN_6 | Nefl | 2.51E−16 |
| PIMN_6 | Popdc3 | 3.30E−16 |
| PIMN_6 | Gfra1 | 3.32E−16 |
| PIMN_6 | Galnt7 | 7.59E−16 |
| PIMN_6 | Rab17 | 1.02E−15 |
| PIMN_6 | Igsf21 | 1.06E−15 |
| PIMN_6 | Grb14 | 1.12E−15 |
| PIMN_6 | Tubb5 | 1.64E−15 |
| PIMN_6 | Sorcs2 | 2.53E−15 |
| PIMN_6 | Tmem255b | 2.57E−15 |
| PIMN_6 | Pcsk1n | 4.23E−15 |
| PIMN_6 | Kctd12 | 4.74E−15 |
| PIMN_6 | Slc1a1 | 5.34E−15 |
| PIMN_6 | Oaz1 | 5.53E−15 |
| PIMN_6 | Kcnq4 | 7.22E−15 |
| PIMN_6 | Cobll1 | 1.55E−14 |
| PIMN_6 | P2ry6 | 1.87E−14 |
| PIMN_6 | Asic4 | 1.92E−14 |
| PIMN_6 | Gm4907 | 2.20E−14 |
| PIMN_6 | Gm12504 | 2.47E−14 |
| PIMN_6 | Fxyd6 | 2.71E−14 |
| PIMN_6 | Map1b | 2.71E−14 |
| PIMN_6 | Hspa2 | 3.16E−14 |
| PIMN_6 | Rarb | 3.89E−14 |
| PIMN_6 | Scd1 | 6.99E−14 |
| PIMN_6 | Gm11747 | 5.50E−13 |
| PIMN_6 | C1qtnf1 | 1.76E−12 |
| PIMN_6 | Ugt1a2 | 5.31E−12 |
| PIMN_6 | Myoz3 | 1.12E−11 |
| PIMN_6 | Kcnv1 | 3.15E−11 |
| PIMN_6 | Sec14l3 | 8.57E−10 |
| PIMN_6 | Adra1d | 2.77E−09 |
| PIMN_6 | Pcdh20 | 3.51E−09 |
| PIMN_6 | Nxph4 | 5.85E−09 |
| PIMN_6 | Aif1l | 8.63E−09 |
| PIMN_6 | Defb48 | 9.39E−08 |
| PIMN_6 | Gareml | 1.77E−07 |
| PIMN_6 | Fam162b | 6.95E−07 |
| PIMN_6 | Lgi3 | 7.04E−07 |
| PIMN_6 | Gjb4 | 7.15E−07 |
| PIMN_6 | Cyp4a31 | 1.00E−06 |
| PIMN_6 | Frat1 | 3.11E−06 |
| PIMN_6 | Gata2 | 4.07E−06 |
| PIMN_6 | Gm14139 | 6.41E−06 |
| PIMN_6 | Omg | 7.40E−06 |
| PIMN_6 | Dpep2 | 9.79E−06 |
| PIMN_6 | Stfa2l1 | 2.44E−05 |
| PIMN_6 | Sult5a1 | 3.15E−05 |
| PIMN_6 | Tmem89 | 3.84E−05 |
| PIMN_6 | Mc1r | 1.31E−04 |
| PIMN_6 | Gpr88 | 1.31E−04 |
| PIMN_6 | Panx3 | 1.61E−04 |
| PIMN_6 | Fcer1a | 1.61E−04 |
| PIMN_6 | Cd1d2 | 3.46E−04 |
| PIMN_6 | Agtrap | 4.68E−04 |
| PIMN_6 | Blk | 6.90E−04 |
| PIMN_6 | Avpr1b | 1.08E−03 |
| PIMN_6 | Actn3 | 1.10E−03 |
| PIMN_6 | Adra2c | 1.11E−03 |
| PIMN_6 | 1700055N04Rik | 1.26E−03 |
| PIMN_6 | Gm11648 | 1.68E−03 |
| PIMN_6 | Tlcd2 | 2.83E−03 |
| PIMN_6 | Gm53 | 2.99E−03 |
| PIMN_6 | Dpys | 3.14E−03 |
| PIMN_6 | Cdh15 | 3.54E−03 |
| PIMN_6 | Nrk | 3.84E−03 |
| PIMN_6 | Gpr182 | 4.26E−03 |
| PIMN_6 | Klhl34 | 4.86E−03 |
| PIMN_6 | Tcf21 | 5.55E−03 |
| PIMN_6 | Lgals2 | 5.98E−03 |
| PIMN_6 | Prl7d1 | 6.91E−03 |
| PIMN_6 | Pik3ap1 | 7.75E−03 |
| PIMN_6 | Gm5039 | 7.78E−03 |
| PIMN_6 | Pgk2 | 9.52E−03 |
| PIMN_6 | Arl4d | 1.02E−02 |
| PIMN_6 | Fsd2 | 1.10E−02 |
| PIMN_6 | Gm12409 | 1.24E−02 |
| PIMN_6 | Pldi | 1.24E−02 |
| PIMN_6 | Cxcl13 | 1.59E−02 |
| PIMN_6 | 4930500F04Rik | 1.63E−02 |
| PIMN_6 | Foxr2 | 1.71E−02 |
| PIMN_6 | Mxd3 | 1.74E−02 |
| PIMN_6 | Klk13 | 1.83E−02 |
| PIMN_6 | Gm16548 | 1.94E−02 |
| PIMN_6 | Rgs2 | 2.37E−02 |
| PIMN_6 | Adam24 | 2.50E−02 |
| PIMN_6 | 2610318N02Rik | 2.53E−02 |
| PIMN_6 | Prf1 | 2.67E−02 |
| PIMN_6 | Derl3 | 2.73E−02 |
| PIMN_6 | Mblac1 | 2.88E−02 |
| PIMN_6 | 4930471C04Rik | 3.61E−02 |
| PIMN_6 | Sex | 3.86E−02 |
| PIMN_6 | Stc2 | 3.87E−02 |
| PIMN_6 | Pcdhb2 | 3.88E−02 |
| PIMN_6 | Hyal1 | 4.15E−02 |
| PIMN_7 | Adarb2 | 2.61E−102 |
| PIMN_7 | Grik3 | 1.44E−43 |
| PIMN_7 | 2610028E06Rik | 4.01E−42 |
| PIMN_7 | Wfdc1 | 1.32E−36 |
| PIMN_7 | Cyp2a5 | 3.94E−33 |
| PIMN_7 | Sstr2 | 1.34E−31 |
| PIMN_7 | Pde1a | 3.93E−29 |
| PIMN_7 | Vip | 1.55E−26 |
| PIMN_7 | Ntng1 | 1.05E−25 |
| PIMN_7 | Lhfp | 1.14E−23 |
| PIMN_7 | 5530401A14Rik | 1.52E−21 |
| PIMN_7 | Prkg2 | 3.64E−21 |
| PIMN_7 | Pdgfd | 4.09E−21 |
| PIMN_7 | Pear1 | 9.88E−21 |
| PIMN_7 | Chrm3 | 1.36E−20 |
| PIMN_7 | Etl4 | 3.49E−20 |
| PIMN_7 | Ebf1 | 1.16E−19 |
| PIMN_7 | Plekhg1 | 2.22E−19 |
| PIMN_7 | Enthd1 | 1.21E−18 |
| PIMN_7 | Asic2 | 1.59E−17 |
| PIMN_7 | Tmem132d | 1.21E−16 |
| PIMN_7 | Ccr5 | 5.38E−16 |
| PIMN_7 | Syt10 | 9.17E−16 |
| PIMN_7 | Creb5 | 1.41E−15 |
| PIMN_7 | A830018L16Rik | 4.43E−15 |
| PIMN_7 | Cbln4 | 8.91E−15 |
| PIMN_7 | Asl | 2.38E−14 |
| PIMN_7 | 1700029J03Rik | 2.38E−14 |
| PIMN_7 | Camk4 | 5.09E−14 |
| PIMN_7 | Chst15 | 5.60E−14 |
| PIMN_7 | Ldb2 | 6.34E−14 |
| PIMN_7 | Casr | 1.10E−13 |
| PIMN_7 | Jazf1 | 1.68E−13 |
| PIMN_7 | Sparcl1 | 1.81E−13 |
| PIMN_7 | Ltk | 1.92E−13 |
| PIMN_7 | Dnahc1 | 4.17E−13 |
| PIMN_7 | Prkd1 | 4.17E−13 |
| PIMN_7 | Sorcs2 | 9.23E−13 |
| PIMN_7 | Rxfp3 | 1.15E−12 |
| PIMN_7 | Pcdh19 | 1.16E−12 |
| PIMN_7 | Sema6a | 1.86E−12 |
| PIMN_7 | Psg22 | 2.24E−12 |
| PIMN_7 | Kcng4 | 2.53E−12 |
| PIMN_7 | Rgs6 | 3.71E−12 |
| PIMN_7 | Krt23 | 3.75E−12 |
| PIMN_7 | Lamb1 | 5.09E−12 |
| PIMN_7 | Lama4 | 5.09E−12 |
| PIMN_7 | Trhde | 6.66E−12 |
| PIMN_7 | Cmah | 7.41E−12 |
| PIMN_7 | Adamts12 | 7.97E−12 |
| PIMN_7 | Frmpd1 | 1.19E−11 |
| PIMN_7 | Robo2 | 1.78E−11 |
| PIMN_7 | Gsg1l | 2.11E−11 |
| PIMN_7 | Dkk3 | 2.33E−11 |
| PIMN_7 | Rarb | 3.50E−11 |
| PIMN_7 | Matn4 | 5.06E−11 |
| PIMN_7 | Vat1l | 6.41E−11 |
| PIMN_7 | Vmn2r-ps54 | 7.79E−11 |
| PIMN_7 | Fam159a | 8.60E−11 |
| PIMN_7 | Gm20757 | 9.29E−11 |
| PIMN_7 | Kcnv1 | 1.72E−10 |
| PIMN_7 | Deptor | 2.23E−10 |
| PIMN_7 | Evpl | 2.27E−10 |
| PIMN_7 | Iqsec3 | 2.33E−10 |
| PIMN_7 | Nosl | 3.81E−10 |
| PIMN_7 | Gm21949 | 3.90E−10 |
| PIMN_7 | Gnb3 | 5.71E−10 |
| PIMN_7 | Kirrel3 | 7.96E−10 |
| PIMN_7 | Tmc3 | 1.29E−09 |
| PIMN_7 | Adamts17 | 1.36E−09 |
| PIMN_7 | Ngf | 2.41E−09 |
| PIMN_7 | Slc7a3 | 4.26E−09 |
| PIMN_7 | Nsg2 | 6.68E−09 |
| PIMN_7 | Stk32a | 7.18E−09 |
| PIMN_7 | Mfsd4 | 7.80E−09 |
| PIMN_7 | Camp | 8.86E−09 |
| PIMN_7 | Serpini1 | 1.33E−08 |
| PIMN_7 | Col25a1 | 1.63E−08 |
| PIMN_7 | BC080695 | 1.79E−08 |
| PIMN_7 | Cntnap5b | 3.21E−08 |
| PIMN_7 | Ass1 | 3.53E−08 |
| PIMN_7 | Slc44a5 | 4.06E−08 |
| PIMN_7 | Dagla | 4.31E−08 |
| PIMN_7 | Pde8b | 4.32E−08 |
| PIMN_7 | Stom | 4.80E−08 |
| PIMN_7 | Grid1 | 4.80E−08 |
| PIMN_7 | Chst11 | 6.35E−08 |
| PIMN_7 | Nav2 | 8.12E−08 |
| PIMN_7 | AW549542 | 1.07E−07 |
| PIMN_7 | Plch2 | 1.16E−07 |
| PIMN_7 | Crtac1 | 1.30E−07 |
| PIMN_7 | Kcnq5 | 1.49E−07 |
| PIMN_7 | Synm | 1.67E−07 |
| PIMN_7 | Kctd1 | 1.73E−07 |
| PIMN_7 | Ngb | 2.06E−07 |
| PIMN_7 | Ngfr | 2.65E−07 |
| PIMN_7 | Prokr1 | 2.73E−07 |
| PIMN_7 | Postn | 3.09E−07 |
| PIMN_7 | Dhrs3 | 3.30E−07 |
| PIMN_7 | Sh3pxd2a | 4.30E−07 |
| PIMN_7 | Igfbp5 | 5.11E−07 |
| PIMN_7 | Ptgir | 6.82E−07 |
| PIMN_7 | Pdyn | 8.57E−07 |
| PIMN_7 | Vwf | 1.42E−06 |
| PIMN_7 | Allc | 3.74E−06 |
| PIMN_7 | 9430076C15Rik | 4.53E−06 |
| PIMN_7 | Apoa2 | 6.46E−06 |
| PIMN_7 | Serpina3g | 8.39E−06 |
| PIMN_7 | Clec1a | 2.25E−05 |
| PIMN_7 | Gm20597 | 2.38E−05 |
| PIMN_7 | 4930556M19Rik | 3.08E−05 |
| PIMN_7 | Rbpjl | 3.81E−05 |
| PIMN_7 | 2810055G20Rik | 7.24E−05 |
| PIMN_7 | Tekt3 | 7.31E−05 |
| PIMN_7 | Cd97 | 7.62E−05 |
| PIMN_7 | Nov | 9.10E−05 |
| PIMN_7 | Serpinb3b | 9.92E−05 |
| PIMN_7 | Calcoco2 | 1.22E−04 |
| PIMN_7 | CK137956 | 1.76E−04 |
| PIMN_7 | Atp6ap1l | 2.06E−04 |
| PIMN_7 | Apol8 | 2.76E−04 |
| PIMN_7 | Prss35 | 3.42E−04 |
| PIMN_7 | Timeless | 5.71E−04 |
| PIMN_7 | Neurl3 | 5.73E−04 |
| PIMN_7 | Omp | 7.12E−04 |
| PIMN_7 | Gpr119 | 7.55E−04 |
| PIMN_7 | F10 | 1.12E−03 |
| PIMN_7 | Khdc1b | 1.55E−03 |
| PIMN_7 | Gm12185 | 1.89E−03 |
| PIMN_7 | Kcnj9 | 1.96E−03 |
| PIMN_7 | Afm | 1.98E−03 |
| PIMN_7 | Myrf | 2.05E−03 |
| PIMN_7 | Kank4 | 2.30E−03 |
| PIMN_7 | Gpr150 | 3.89E−03 |
| PIMN_7 | Mroh4 | 4.96E−03 |
| PIMN_7 | Htr2a | 5.36E−03 |
| PIMN_7 | Hmga2 | 6.11E−03 |
| PIMN_7 | Vmn2r106 | 6.11E−03 |
| PIMN_7 | Adra2c | 7.66E−03 |
| PIMN_7 | Slc23a3 | 1.04E−02 |
| PIMN_7 | Smim18 | 1.05E−02 |
| PIMN_7 | Capza3 | 1.11E−02 |
| PIMN_7 | Hoxa11 | 1.17E−02 |
| PIMN_7 | A530046M15Rik | 1.27E−02 |
| PIMN_7 | Gm10494 | 1.43E−02 |
| PIMN_7 | Plcg2 | 1.59E−02 |
| PIMN_7 | Clec9a | 1.96E−02 |
| PIMN_7 | Retn | 1.98E−02 |
| PIMN_7 | Gal3st1 | 2.03E−02 |
| PIMN_7 | Hepacam | 2.76E−02 |
| PIMN_7 | Cd300e | 3.20E−02 |
| PIMN_7 | Gm438 | 4.55E−02 |
| PIN_1 | Pde7b | 0.00E+00 |
| PIN_1 | Camk1d | 0.00E+00 |
| PIN_1 | Sema3e | 1.67E−292 |
| PIN_1 | L3mbtl4 | 7.38E−285 |
| PIN_1 | Kctd16 | 1.45E−268 |
| PIN_1 | Eepd1 | 1.15E−187 |
| PIN_1 | Dock1 | 2.86E−161 |
| PIN_1 | Prr16 | 1.66E−149 |
| PIN_1 | Shisa6 | 2.82E−148 |
| PIN_1 | Mgll | 5.52E−136 |
| PIN_1 | Sema5b | 1.84E−135 |
| PIN_1 | Egflam | 8.88E−134 |
| PIN_1 | Stac | 1.60E−132 |
| PIN_1 | Dlgap1 | 6.65E−132 |
| PIN_1 | Nfatc1 | 3.40E−130 |
| PIN_1 | Met | 3.92E−129 |
| PIN_1 | Lamc3 | 1.48E−124 |
| PIN_1 | Leprel1 | 3.35E−123 |
| PIN_1 | Fam189a1 | 8.51E−123 |
| PIN_1 | Slc24a2 | 6.21E−122 |
| PIN_1 | Nckap5 | 6.80E−121 |
| PIN_1 | Grm8 | 1.29E−117 |
| PIN_1 | Grm7 | 1.97E−115 |
| PIN_1 | Lingo2 | 9.45E−114 |
| PIN_1 | Fras1 | 5.71E−103 |
| PIN_1 | Mir466d | 2.74E−100 |
| PIN_1 | Fut9 | 1.46E−99 |
| PIN_1 | Ntn1 | 1.33E−95 |
| PIN_1 | Col27a1 | 1.45E−95 |
| PIN_1 | Fibcd1 | 5.41E−95 |
| PIN_1 | Inpp4b | 8.46E−95 |
| PIN_1 | Dapk1 | 1.60E−94 |
| PIN_1 | Egfr | 6.15E−93 |
| PIN_1 | Khdrbs3 | 4.23E−91 |
| PIN_1 | Gm20754 | 7.91E−91 |
| PIN_1 | Wnk4 | 8.93E−89 |
| PIN_1 | 2900055J20Rik | 5.93E−87 |
| PIN_1 | Egfl6 | 7.30E−87 |
| PIN_1 | Cadm2 | 9.52E−84 |
| PIN_1 | Hcn1 | 2.54E−82 |
| PIN_1 | Grid1 | 1.75E−81 |
| PIN_1 | Flrt2 | 2.44E−81 |
| PIN_1 | Map2 | 9.18E−81 |
| PIN_1 | Pitpnc1 | 1.61E−79 |
| PIN_1 | Tac1 | 9.28E−77 |
| PIN_1 | Lmo7 | 9.28E−77 |
| PIN_1 | Gm1604b | 1.09E−76 |
| PIN_1 | Galr1 | 7.54E−76 |
| PIN_1 | Pbx3 | 1.92E−75 |
| PIN_1 | Tmtc1 | 8.99E−74 |
| PIN_1 | Skap1 | 2.87E−73 |
| PIN_1 | Ror2 | 1.50E−71 |
| PIN_1 | Ppp3ca | 1.65E−71 |
| PIN_1 | Col8a1 | 1.93E−70 |
| PIN_1 | Snx7 | 3.05E−70 |
| PIN_1 | Cldn11 | 9.35E−69 |
| PIN_1 | Shisa9 | 2.19E−68 |
| PIN_1 | Epb4.1l4a | 2.10E−67 |
| PIN_1 | Pde4d | 4.44E−67 |
| PIN_1 | Phactr1 | 8.97E−67 |
| PIN_1 | Prlr | 9.36E−67 |
| PIN_1 | Gucy2g | 7.98E−66 |
| PIN_1 | Chrm3 | 7.69E−63 |
| PIN_1 | Prkg1 | 1.75E−62 |
| PIN_1 | Nos1ap | 1.95E−62 |
| PIN_1 | Pbx1 | 2.79E−62 |
| PIN_1 | Calcr1 | 1.51E−61 |
| PIN_1 | Pdia5 | 1.69E−61 |
| PIN_1 | Fam126a | 2.10E−61 |
| PIN_1 | Kctd8 | 4.82E−61 |
| PIN_1 | Zfhx3 | 3.62E−60 |
| PIN_1 | Cnksr2 | 5.61E−59 |
| PIN_1 | Fam196a | 5.51E−58 |
| PIN_1 | 4930509J09Rik | 3.34E−57 |
| PIN_1 | Cask | 4.98E−57 |
| PIN_1 | Enpp2 | 2.95E−55 |
| PIN_1 | Tenm4 | 1.89E−54 |
| PIN_1 | Tmc3 | 2.41E−54 |
| PIN_1 | Kirrel3 | 9.91E−54 |
| PIN_1 | Fam107b | 8.82E−52 |
| PIN_1 | Sptb | 4.98E−51 |
| PIN_1 | Stxbp5l | 5.81E−51 |
| PIN_1 | Plcl1 | 1.61E−50 |
| PIN_1 | Fam19a5 | 3.85E−50 |
| PIN_1 | Boc | 5.39E−50 |
| PIN_1 | Ptprz1 | 1.02E−49 |
| PIN_1 | Slitrk4 | 1.49E−49 |
| PIN_1 | Bicc1 | 5.21E−49 |
| PIN_1 | Nhs | 4.00E−48 |
| PIN_1 | Mast4 | 1.91E−47 |
| PIN_1 | Kcnh5 | 7.11E−47 |
| PIN_1 | Sez6l | 5.42E−46 |
| PIN_1 | Abcc8 | 1.44E−45 |
| PIN_1 | Dock2 | 2.06E−45 |
| PIN_1 | Atp1a3 | 2.14E−45 |
| PIN_1 | Crim1 | 9.39E−45 |
| PIN_1 | Fam196b | 2.09E−44 |
| PIN_1 | Phactr2 | 4.27E−44 |
| PIN_1 | Ggta1 | 1.90E−43 |
| PIN_1 | Aff3 | 1.70E−42 |
| PIN_1 | Sparcl1 | 8.76E−42 |
| PIN_1 | Hsd11b1 | 3.98E−40 |
| PIN_1 | 4930578E11Rik | 6.85E−40 |
| PIN_1 | Mtnr1a | 2.67E−32 |
| PIN_1 | Ramp2 | 1.70E−29 |
| PIN_1 | Gm12171 | 7.07E−28 |
| PIN_1 | Gcsam | 2.80E−27 |
| PIN_1 | Bmp6 | 9.80E−27 |
| PIN_1 | 2810011L19Rik | 3.97E−26 |
| PIN_1 | Col5a1 | 9.66E−18 |
| PIN_1 | Kirrel2 | 3.34E−17 |
| PIN_1 | Sfrp2 | 4.22E−17 |
| PIN_1 | 4933416E03Rik | 5.83E−15 |
| PIN_1 | Pcdh8 | 1.66E−12 |
| PIN_1 | Cenph | 1.47E−11 |
| PIN_1 | Sostdc1 | 1.55E−11 |
| PIN_1 | Gm17745 | 1.69E−11 |
| PIN_1 | 6720468P15Rik | 3.65E−11 |
| PIN_1 | Lrrc18 | 2.52E−10 |
| PIN_1 | Ces2b | 3.78E−10 |
| PIN_1 | Zfp831 | 2.84E−09 |
| PIN_1 | 4932435O22Rik | 1.00E−08 |
| PIN_1 | Cd300a | 2.37E−08 |
| PIN_1 | Ibsp | 6.01E−08 |
| PIN_1 | Rbp7 | 7.29E−08 |
| PIN_1 | Gm826 | 1.09E−07 |
| PIN_1 | Tectb | 1.14E−07 |
| PIN_1 | Gngt2 | 1.15E−07 |
| PIN_1 | Kng1 | 5.46E−07 |
| PIN_1 | Ntrk1 | 6.63E−07 |
| PIN_1 | 9130015L21Rik | 7.51E−07 |
| PIN_1 | Kcna3 | 1.36E−06 |
| PIN_1 | Ccl7 | 2.22E−06 |
| PIN_1 | Nphs1as | 3.40E−06 |
| PIN_1 | 4932411E22Rik | 4.20E−06 |
| PIN_1 | Cxcr4 | 7.12E−06 |
| PIN_1 | Gm13119 | 2.92E−05 |
| PIN_1 | 1700034G24Rik | 3.00E−05 |
| PIN_1 | Lox | 4.05E−05 |
| PIN_1 | Pla2g1b | 7.44E−05 |
| PIN_1 | Hoxd8 | 7.96E−05 |
| PIN_1 | 4930596D02Rik | 1.09E−04 |
| PIN_1 | Ces1b | 1.46E−04 |
| PIN_1 | Trem3 | 2.34E−04 |
| PIN_1 | Angptl4 | 2.67E−04 |
| PIN_1 | Hoxd1 | 3.32E−04 |
| PIN_1 | BC055402 | 4.15E−04 |
| PIN_1 | Prnd | 6.82E−04 |
| PIN_1 | Bsx | 7.95E−04 |
| PIN_1 | 1700061l17Rik | 1.05E−03 |
| PIN_1 | Nptx2 | 1.40E−03 |
| PIN_1 | 4930500F04Rik | 2.00E−03 |
| PIN_1 | Aadacl2 | 2.08E−03 |
| PIN_1 | Srpx2 | 3.87E−03 |
| PIN_1 | Gabrq | 4.15E−03 |
| PIN_1 | Pla2g2d | 6.63E−03 |
| PIN_1 | Fcgr2b | 7.56E−03 |
| PIN_1 | Ptges | 9.90E−03 |
| PIN_1 | Notum | 1.21E−02 |
| PIN_1 | Ccl11 | 1.30E−02 |
| PIN_1 | Lin28a | 1.32E−02 |
| PIN_1 | Lrrc52 | 2.24E−02 |
| PIN_1 | Slamf8 | 2.46E−02 |
| PIN_1 | Rhox5 | 2.55E−02 |
| PIN_1 | Mageb3 | 2.90E−02 |
| PIN_1 | Gm11346 | 4.52E−02 |
| PIN_2 | Fut9 | 1.19E−67 |
| PIN_2 | Ptger2 | 7.58E−64 |
| PIN_2 | Penk | 3.51E−59 |
| PIN_2 | Gm20754 | 3.53E−59 |
| PIN_2 | Tac1 | 4.57E−58 |
| PIN_2 | Nfatc1 | 1.54E−55 |
| PIN_2 | Egfr | 1.79E−54 |
| PIN_2 | Lamc3 | 5.00E−49 |
| PIN_2 | Cd200 | 7.97E−48 |
| PIN_2 | Lingo2 | 1.51E−44 |
| PIN_2 | Pde4d | 2.89E−44 |
| PIN_2 | Car8 | 1.17E−43 |
| PIN_2 | Ntrk2 | 1.99E−41 |
| PIN_2 | Ptprz1 | 6.25E−37 |
| PIN_2 | Col27a1 | 2.56E−36 |
| PIN_2 | Stac | 2.60E−36 |
| PIN_2 | Rgs4 | 3.66E−35 |
| PIN_2 | Nsg1 | 4.91E−35 |
| PIN_2 | Pitpnc1 | 1.45E−33 |
| PIN_2 | Kctd16 | 1.90E−33 |
| PIN_2 | Slc10a4 | 1.54E−32 |
| PIN_2 | Psmd1 | 6.39E−32 |
| PIN_2 | Pde7b | 3.25E−31 |
| PIN_2 | Unc5d | 5.46E−31 |
| PIN_2 | 4930509J09Rik | 1.35E−30 |
| PIN_2 | Skap1 | 2.04E−30 |
| PIN_2 | Jph1 | 1.04E−29 |
| PIN_2 | Gm5868 | 2.00E−29 |
| PIN_2 | Kctd8 | 2.07E−28 |
| PIN_2 | Gucy2g | 8.42E−28 |
| PIN_2 | Dlgap1 | 1.32E−27 |
| PIN_2 | Leprel1 | 1.60E−27 |
| PIN_2 | Abcc8 | 5.78E−27 |
| PIN_2 | Itgb8 | 6.60E−27 |
| PIN_2 | 1810006J02Rik | 1.10E−26 |
| PIN_2 | Kl | 2.43E−26 |
| PIN_2 | Mgll | 3.75E−25 |
| PIN_2 | Sstr1 | 4.19E−25 |
| PIN_2 | Galr1 | 5.26E−25 |
| PIN_2 | Ust | 1.04E−24 |
| PIN_2 | Tmem132e | 1.50E−24 |
| PIN_2 | Nhsl2 | 3.09E−24 |
| PIN_2 | Htr2b | 3.97E−24 |
| PIN_2 | Dock10 | 3.97E−24 |
| PIN_2 | Fras1 | 4.19E−24 |
| PIN_2 | Thbs1 | 1.33E−22 |
| PIN_2 | Gpr64 | 1.51E−22 |
| PIN_2 | Slc12a2 | 2.56E−22 |
| PIN_2 | Thsd4 | 6.03E−22 |
| PIN_2 | Siglec15 | 7.36E−22 |
| PIN_2 | Whrn | 1.59E−21 |
| PIN_2 | 5530401A14Rik | 1.95E−21 |
| PIN_2 | Fam19a5 | 2.77E−21 |
| PIN_2 | Dnaja1 | 8.38E−21 |
| PIN_2 | Proser2 | 1.37E−20 |
| PIN_2 | Pbx3 | 1.58E−20 |
| PIN_2 | Tmc3 | 2.95E−20 |
| PIN_2 | Rwdd3 | 4.12E−20 |
| PIN_2 | Hoxb5 | 6.02E−20 |
| PIN_2 | Psmd13 | 1.76E−19 |
| PIN_2 | Grm7 | 4.65E−19 |
| PIN_2 | Snx7 | 5.16E−19 |
| PIN_2 | Parva | 5.60E−19 |
| PIN_2 | Cd109 | 1.10E−18 |
| PIN_2 | Gda | 1.35E−18 |
| PIN_2 | 2900055J20Rik | 2.28E−18 |
| PIN_2 | Mbp | 4.45E−18 |
| PIN_2 | Fibcd1 | 5.22E−18 |
| PIN_2 | Vmn2r28 | 5.22E−18 |
| PIN_2 | Fjx1 | 6.83E−18 |
| PIN_2 | Galnt9 | 1.10E−17 |
| PIN_2 | Prkg1 | 1.68E−17 |
| PIN_2 | Cntn5 | 1.80E−17 |
| PIN_2 | Bnc2 | 1.81E−17 |
| PIN_2 | Ldlrad3 | 9.12E−17 |
| PIN_2 | Scg3 | 1.39E−16 |
| PIN_2 | Gm19782 | 1.41E−16 |
| PIN_2 | Gm10440 | 1.45E−16 |
| PIN_2 | Epdr1 | 1.58E−16 |
| PIN_2 | L3mbtl4 | 2.92E−16 |
| PIN_2 | Cntn6 | 3.69E−16 |
| PIN_2 | Bicc1 | 5.46E−16 |
| PIN_2 | Nhs | 6.32E−16 |
| PIN_2 | Arhgap28 | 7.59E−16 |
| PIN_2 | Nrp2 | 7.90E−16 |
| PIN_2 | Ptk2b | 1.07E−15 |
| PIN_2 | Atp2b4 | 1.21E−15 |
| PIN_2 | Prkcb | 1.56E−15 |
| PIN_2 | Tagln3 | 2.15E−15 |
| PIN_2 | Kirrel3 | 3.48E−15 |
| PIN_2 | Arhgef3 | 3.64E−15 |
| PIN_2 | Tgfb1i1 | 5.72E−15 |
| PIN_2 | Slitrk4 | 7.27E−15 |
| PIN_2 | Sorbs2 | 1.21E−14 |
| PIN_2 | Asic2 | 1.49E−14 |
| PIN_2 | Txndc16 | 1.76E−14 |
| PIN_2 | Pfn2 | 2.68E−14 |
| PIN_2 | A730046J19Rik | 3.37E−14 |
| PIN_2 | Fxyd7 | 3.62E−14 |
| PIN_2 | Il22ra1 | 7.93E−14 |
| PIN_2 | Itih3 | 8.03E−13 |
| PIN_2 | Slco4c1 | 5.94E−12 |
| PIN_2 | BC051537 | 1.58E−10 |
| PIN_2 | Trim71 | 2.71E−10 |
| PIN_2 | Ptgdr | 2.61E−09 |
| PIN_2 | BC055402 | 3.03E−09 |
| PIN_2 | 4833428L15Rik | 3.83E−09 |
| PIN_2 | Bpifa3 | 4.03E−09 |
| PIN_2 | Gm13277 | 1.81E−08 |
| PIN_2 | Ripply3 | 5.41E−08 |
| PIN_2 | Tectb | 3.00E−07 |
| PIN_2 | Lyzl6 | 7.20E−07 |
| PIN_2 | Ctxn3 | 8.56E−07 |
| PIN_2 | AA387883 | 1.07E−06 |
| PIN_2 | Zfp474 | 1.39E−06 |
| PIN_2 | C1ql2 | 1.41E−06 |
| PIN_2 | Vmn2rl22 | 1.99E−06 |
| PIN_2 | Vmn2r94 | 3.85E−06 |
| PIN_2 | 9830107B12Rik | 5.67E−06 |
| PIN_2 | 4930431P03Rik | 6.73E−06 |
| PIN_2 | Spesp1 | 1.09E−05 |
| PIN_2 | Crabp2 | 1.48E−05 |
| PIN_2 | Slc30a2 | 1.79E−05 |
| PIN_2 | Btla | 1.93E−05 |
| PIN_2 | AI607873 | 2.31E−05 |
| PIN_2 | Mag | 2.68E−05 |
| PIN_2 | Gm4567 | 3.90E−05 |
| PIN_2 | Slco1a5 | 4.80E−05 |
| PIN_2 | Ramp2 | 6.89E−05 |
| PIN_2 | Fzd2 | 8.02E−05 |
| PIN_2 | Gm11240 | 1.38E−04 |
| PIN_2 | Ctcfl | 1.39E−04 |
| PIN_2 | Klf17 | 1.48E−04 |
| PIN_2 | Hbb-b1 | 1.71E−04 |
| PIN_2 | Chi3l1 | 2.31E−04 |
| PIN_2 | Nostrin | 2.40E−04 |
| PIN_2 | 4930404H11Rik | 2.55E−04 |
| PIN_2 | Gm17745 | 2.79E−04 |
| PIN_2 | Mog | 3.97E−04 |
| PIN_2 | 4930564D02Rik | 4.34E−04 |
| PIN_2 | Krt74 | 4.37E−04 |
| PIN_2 | D16Ertd519e | 4.40E−04 |
| PIN_2 | 1700108F19Rik | 4.45E−04 |
| PIN_2 | Eve | 4.46E−04 |
| PIN_2 | Cdh3 | 6.58E−04 |
| PIN_2 | LOC100504608 | 1.13E−03 |
| PIN_2 | Vmn2r67 | 1.24E−03 |
| PIN_2 | E030044B06Rik | 1.31E−03 |
| PIN_2 | Duoxa1 | 1.33E−03 |
| PIN_2 | Cyp26a1 | 1.68E−03 |
| PIN_2 | Gm826 | 1.70E−03 |
| PIN_2 | Gm2762 | 2.33E−03 |
| PIN_2 | Aifm3 | 2.82E−03 |
| PIN_2 | Cxcr4 | 2.97E−03 |
| PIN_2 | Ankk1 | 3.36E−03 |
| PIN_2 | Trim75 | 5.30E−03 |
| PIN_2 | Ddit4l | 5.85E−03 |
| PIN_2 | 2310015B20Rik | 7.10E−03 |
| PIN_2 | A330070K13Rik | 7.30E−03 |
| PIN_2 | AI847159 | 7.53E−03 |
| PIN_2 | BC049635 | 7.79E−03 |
| PIN_2 | Hmox1 | 8.29E−03 |
| PIN_2 | Myh2 | 8.60E−03 |
| PIN_2 | 2210409D07Rik | 8.72E−03 |
| PIN_2 | Mrgprb1 | 1.10E−02 |
| PIN_2 | Ccl2 | 1.19E−02 |
| PIN_2 | 1700054A03Rik | 1.28E−02 |
| PIN_2 | Adam33 | 1.68E−02 |
| PIN_2 | Cxcl14 | 1.92E−02 |
| PIN_2 | Agtr2 | 2.77E−02 |
| PIN_2 | Gm13032 | 2.84E−02 |
| PIN_2 | Vmn1r3 | 3.28E−02 |
| PIN_2 | Clec1b | 3.58E−02 |
| PIN_2 | Hmgn5 | 4.22E−02 |
| PIN_3 | Gna14 | 0.00E+00 |
| PIN_3 | Nxph2 | 0.00E+00 |
| PIN_3 | Klhl1 | 0.00E+00 |
| PIN_3 | Ano5 | 1.22E−204 |
| PIN_3 | Ntng1 | 2.56E−175 |
| PIN_3 | Zmat4 | 6.93E−164 |
| PIN_3 | Kif26b | 2.16E−148 |
| PIN_3 | Tmeff2 | 1.01E−133 |
| PIN_3 | Csmd1 | 1.46E−124 |
| PIN_3 | Slc17a6 | 1.76E−116 |
| PIN_3 | Galnt18 | 3.57E−116 |
| PIN_3 | Trps1 | 3.57E−116 |
| PIN_3 | Dlc1 | 2.63E−115 |
| PIN_3 | Kcnh7 | 3.38E−96 |
| PIN_3 | Pcp4l1 | 1.52E−91 |
| PIN_3 | Zbbx | 5.62E−87 |
| PIN_3 | Skap1 | 8.33E−87 |
| PIN_3 | Cntn5 | 1.68E−86 |
| PIN_3 | Serpini1 | 2.01E−84 |
| PIN_3 | Ddc | 5.25E−80 |
| PIN_3 | Tenm4 | 7.47E−80 |
| PIN_3 | Flrt2 | 3.34E−76 |
| PIN_3 | Gng2 | 4.77E−74 |
| PIN_3 | Atp7a | 8.93E−74 |
| PIN_3 | Sgcz | 1.99E−73 |
| PIN_3 | Tnr | 3.83E−73 |
| PIN_3 | Olfr78 | 2.76E−72 |
| PIN_3 | 0610009B14Rik | 4.48E−71 |
| PIN_3 | Spock3 | 2.91E−70 |
| PIN_3 | Eif3h | 2.58E−69 |
| PIN_3 | Nefm | 2.05E−67 |
| PIN_3 | Bmpr1b | 2.98E−66 |
| PIN_3 | Penk | 1.84E−62 |
| PIN_3 | Prkca | 3.44E−62 |
| PIN_3 | Kcng1 | 1.42E−61 |
| PIN_3 | Sv2c | 4.17E−61 |
| PIN_3 | Pbx3 | 1.47E−60 |
| PIN_3 | Nefl | 1.56E−60 |
| PIN_3 | Ddah1 | 2.19E−59 |
| PIN_3 | Adcyap1 | 1.18E−57 |
| PIN_3 | Sez6l | 1.30E−57 |
| PIN_3 | Lrrn3 | 2.25E−57 |
| PIN_3 | Arhgap28 | 2.84E−57 |
| PIN_3 | Spock1 | 2.57E−55 |
| PIN_3 | Mir466g | 1.38E−54 |
| PIN_3 | Bcl2 | 2.09E−54 |
| PIN_3 | Nebl | 3.30E−54 |
| PIN_3 | Cd24a | 1.38E−53 |
| PIN_3 | Npy1r | 1.44E−53 |
| PIN_3 | Stac | 1.74E−52 |
| PIN_3 | Pcdh7 | 7.43E−52 |
| PIN_3 | Rasgrf1 | 6.57E−51 |
| PIN_3 | March1 | 8.38E−51 |
| PIN_3 | L3mbtl4 | 9.55E−51 |
| PIN_3 | Onecut2 | 2.03E−49 |
| PIN_3 | Osbpl6 | 4.24E−49 |
| PIN_3 | Fam107b | 7.82E−49 |
| PIN_3 | Nox3 | 1.39E−48 |
| PIN_3 | Tmem44 | 6.71E−48 |
| PIN_3 | D930015E06Rik | 1.68E−47 |
| PIN_3 | 1700042O10Rik | 2.18E−46 |
| PIN_3 | Fam5c | 2.35E−46 |
| PIN_3 | Parva | 3.22E−46 |
| PIN_3 | Sytl5 | 1.06E−45 |
| PIN_3 | Fam19a2 | 1.15E−45 |
| PIN_3 | Mndal | 1.81E−45 |
| PIN_3 | Cdh18 | 2.35E−45 |
| PIN_3 | Mmp17 | 7.91E−45 |
| PIN_3 | Enox1 | 2.82E−43 |
| PIN_3 | Dbh | 5.52E−43 |
| PIN_3 | Cpne8 | 1.27E−42 |
| PIN_3 | Ush1c | 2.48E−41 |
| PIN_3 | 9330175M20Rik | 5.32E−41 |
| PIN_3 | Itm2a | 4.78E−40 |
| PIN_3 | Mfap3l | 5.58E−40 |
| PIN_3 | Meis1 | 9.25E−40 |
| PIN_3 | Cyb561 | 9.40E−40 |
| PIN_3 | Tanc2 | 4.38E−39 |
| PIN_3 | Mt3 | 7.53E−39 |
| PIN_3 | Tshr | 9.11E−39 |
| PIN_3 | Rab27b | 1.00E−38 |
| PIN_3 | Xpr1 | 1.28E−38 |
| PIN_3 | Htr4 | 1.79E−38 |
| PIN_3 | 2610307P16Rik | 1.86E−38 |
| PIN_3 | Epb4.1l4a | 5.98E−38 |
| PIN_3 | 2810471M01Rik | 8.53E−37 |
| PIN_3 | Pde9a | 1.38E−36 |
| PIN_3 | Zfhx3 | 4.36E−36 |
| PIN_3 | Ifi203 | 6.22E−36 |
| PIN_3 | Unc5c | 7.37E−36 |
| PIN_3 | Colq | 8.64E−36 |
| PIN_3 | Apba1 | 8.97E−36 |
| PIN_3 | 1600029O15Rik | 4.03E−35 |
| PIN_3 | Pde4b | 1.28E−34 |
| PIN_3 | Palm2 | 1.79E−34 |
| PIN_3 | Plcl1 | 2.96E−34 |
| PIN_3 | Lpar4 | 1.09E−33 |
| PIN_3 | AW549542 | 1.14E−33 |
| PIN_3 | Islr2 | 1.90E−33 |
| PIN_3 | Fam122b | 1.21E−32 |
| PIN_3 | Gm16065 | 1.34E−25 |
| PIN_3 | Npy5r | 5.23E−25 |
| PIN_3 | Runx1 | 7.56E−24 |
| PIN_3 | Sstr5 | 2.36E−21 |
| PIN_3 | A630033H20Rik | 6.88E−18 |
| PIN_3 | Taarl | 1.57E−15 |
| PIN_3 | 4930556J02Rik | 2.60E−14 |
| PIN_3 | Taar2 | 1.11E−13 |
| PIN_3 | Irf5 | 2.44E−13 |
| PIN_3 | Spp2 | 4.48E−13 |
| PIN_3 | Cd40 | 8.35E−13 |
| PIN_3 | Ankrd34c | 1.32E−12 |
| PIN_3 | 4930598F16Rik | 4.37E−12 |
| PIN_3 | Cckar | 4.69E−12 |
| PIN_3 | Olfr560 | 2.35E−11 |
| PIN_3 | Islr | 8.90E−10 |
| PIN_3 | Rtp1 | 2.65E−09 |
| PIN_3 | Vnn1 | 2.11E−08 |
| PIN_3 | Tmprss13 | 4.90E−08 |
| PIN_3 | Odam | 1.67E−07 |
| PIN_3 | Fbxw28 | 3.92E−07 |
| PIN_3 | Ccdc33 | 7.77E−07 |
| PIN_3 | Samd7 | 9.49E−07 |
| PIN_3 | Efcab8 | 9.89E−07 |
| PIN_3 | Myo1g | 1.95E−06 |
| PIN_3 | Zp2 | 2.04E−06 |
| PIN_3 | Rhox3a | 8.09E−06 |
| PIN_3 | Olfr5 | 8.74E−06 |
| PIN_3 | Pde4c | 2.13E−05 |
| PIN_3 | Taar3 | 2.14E−05 |
| PIN_3 | Slc6a2 | 5.71E−05 |
| PIN_3 | Adra2b | 5.74E−05 |
| PIN_3 | Acsm2 | 9.09E−05 |
| PIN_3 | Prss23 | 1.13E−04 |
| PIN_3 | 1700027A15Rik | 1.18E−04 |
| PIN_3 | Vrtn | 1.57E−04 |
| PIN_3 | Olfr1383 | 2.00E−04 |
| PIN_3 | Hoxb1 | 3.87E−04 |
| PIN_3 | Prl2c2 | 4.79E−04 |
| PIN_3 | 4930513O06Rik | 5.29E−04 |
| PIN_3 | Prss40 | 5.44E−04 |
| PIN_3 | Taar4 | 8.69E−04 |
| PIN_3 | 4930470P17Rik | 9.21E−04 |
| PIN_3 | 2810433D01Rik | 1.48E−03 |
| PIN_3 | 1700021N21Rik | 1.85E−03 |
| PIN_3 | Cd5l | 2.72E−03 |
| PIN_3 | A430089I19Rik | 2.87E−03 |
| PIN_3 | Nr1h5 | 2.99E−03 |
| PIN_3 | Prrx1 | 3.11E−03 |
| PIN_3 | Krtap12-1 | 4.32E−03 |
| PIN_3 | Taar5 | 4.43E−03 |
| PIN_3 | Procr | 6.09E−03 |
| PIN_3 | 4930503007Rik | 6.68E−03 |
| PIN_3 | Prps1l1 | 7.47E−03 |
| PIN_3 | 1500015L24Rik | 8.41E−03 |
| PIN_3 | 6530402F18Rik | 9.58E−03 |
| PIN_3 | Gm10024 | 1.19E−02 |
| PIN_3 | Cldn24 | 1.19E−02 |
| PIN_3 | Serpina4-ps1 | 1.30E−02 |
| PIN_3 | Hoxa13 | 1.64E−02 |
| PIN_3 | Il17c | 2.42E−02 |
| PIN_3 | Zcchc5 | 2.44E−02 |
| PIN_3 | Gm3285 | 2.98E−02 |
| PIN_3 | Unc5cl | 3.60E−02 |
| PIN_3 | 1700095B10Rik | 3.66E−02 |
| PIN_3 | Mir137 | 4.04E−02 |
| PIN_3 | C430002E04Rik | 4.84E−02 |
| PIN_3 | Ms4a15 | 4.87E−02 |
| PSN_1 | Ano2 | 5.87E−212 |
| PSN_1 | Cdh8 | 1.13E−193 |
| PSN_1 | Speer7-ps1 | 1.96E−157 |
| PSN_1 | Mgat4c | 9.12E−133 |
| PSN_1 | Zfp804a | 3.13E−123 |
| PSN_1 | Iqub | 4.81E−117 |
| PSN_1 | Efr3a | 2.72E−112 |
| PSN_1 | Dapk2 | 1.08E−110 |
| PSN_1 | Speer8-ps1 | 1.11E−108 |
| PSN_1 | Itgb6 | 9.47E−94 |
| PSN_1 | Dgkg | 4.49E−89 |
| PSN_1 | Gpr149 | 1.62E−83 |
| PSN_1 | A330076C08Rik | 1.28E−79 |
| PSN_1 | Ccbe1 | 1.71E−78 |
| PSN_1 | Robo2 | 7.01E−77 |
| PSN_1 | Nmu | 1.69E−75 |
| PSN_1 | Rab27b | 1.40E−74 |
| PSN_1 | Grin3a | 2.36E−73 |
| PSN_1 | Arhgap6 | 1.87E−69 |
| PSN_1 | Clstn2 | 4.48E−69 |
| PSN_1 | Cux2 | 5.55E−69 |
| PSN_1 | Tcf7l2 | 1.07E−66 |
| PSN_1 | Cpne4 | 1.96E−60 |
| PSN_1 | Speer5-ps1 | 9.20E−57 |
| PSN_1 | Myl1 | 2.14E−54 |
| PSN_1 | Cbln2 | 3.81E−53 |
| PSN_1 | Ngfr | 7.20E−53 |
| PSN_1 | Cdh6 | 9.77E−52 |
| PSN_1 | Layn | 2.65E−49 |
| PSN_1 | Hpcal1 | 5.69E−49 |
| PSN_1 | Slc2a13 | 9.27E−49 |
| PSN_1 | Scn7a | 4.95E−47 |
| PSN_1 | Pcdh9 | 1.05E−44 |
| PSN_1 | Speer4d | 2.51E−44 |
| PSN_1 | Vgll3 | 1.04E−42 |
| PSN_1 | 4930572O03Rik | 1.62E−42 |
| PSN_1 | Hpca | 2.14E−42 |
| PSN_1 | Pkib | 2.11E−41 |
| PSN_1 | Hspb8 | 2.11E−41 |
| PSN_1 | Prkag2 | 1.37E−39 |
| PSN_1 | Avil | 3.91E−39 |
| PSN_1 | Gm9758 | 4.93E−39 |
| PSN_1 | Tmeff2 | 1.05E−38 |
| PSN_1 | Calcb | 2.41E−38 |
| PSN_1 | Speer4e | 3.27E−38 |
| PSN_1 | Tacr1 | 4.65E−38 |
| PSN_1 | Gm17019 | 1.39E−37 |
| PSN_1 | Apba2 | 1.88E−37 |
| PSN_1 | Agrn | 3.03E−37 |
| PSN_1 | Rph3a | 4.70E−37 |
| PSN_1 | Atoh8 | 2.49E−35 |
| PSN_1 | Il7 | 4.88E−35 |
| PSN_1 | Gcgr | 7.46E−35 |
| PSN_1 | Snx31 | 7.46E−35 |
| PSN_1 | Nrxn3 | 1.99E−34 |
| PSN_1 | Tbx2 | 5.30E−34 |
| PSN_1 | Pak7 | 7.24E−34 |
| PSN_1 | Il13ra1 | 8.99E−34 |
| PSN_1 | Htr3a | 3.61E−33 |
| PSN_1 | Dgki | 1.56E−32 |
| PSN_1 | Galr1 | 5.14E−32 |
| PSN_1 | Ptprt | 1.42E−31 |
| PSN_1 | Nos1ap | 3.00E−31 |
| PSN_1 | Dclk3 | 7.74E−31 |
| PSN_1 | Dlx3 | 8.81E−31 |
| PSN_1 | Gm9199 | 1.29E−30 |
| PSN_1 | B3galt1 | 1.60E−30 |
| PSN_1 | Unc13c | 2.90E−30 |
| PSN_1 | Capn5 | 3.98E−30 |
| PSN_1 | Ntrk3 | 6.86E−30 |
| PSN_1 | Pkia | 3.09E−29 |
| PSN_1 | Smad6 | 8.97E−29 |
| PSN_1 | Grp | 1.40E−28 |
| PSN_1 | Lhfp12 | 2.87E−28 |
| PSN_1 | Gm12530 | 3.33E−28 |
| PSN_1 | Greb1 | 1.62E−27 |
| PSN_1 | Met | 1.68E−27 |
| PSN_1 | Spock3 | 2.63E−27 |
| PSN_1 | 1700007B14Rik | 6.30E−27 |
| PSN_1 | Cachd1 | 2.96E−26 |
| PSN_1 | Slc12a7 | 4.27E−26 |
| PSN_1 | Dnaja1 | 5.85E−26 |
| PSN_1 | Gstm1 | 6.53E−26 |
| PSN_1 | Spag5 | 7.05E−26 |
| PSN_1 | Spsb1 | 7.45E−26 |
| PSN_1 | Psmd13 | 9.77E−26 |
| PSN_1 | Hspb1 | 4.93E−25 |
| PSN_1 | Cntnap3 | 5.97E−25 |
| PSN_1 | Pcgf1 | 2.95E−24 |
| PSN_1 | Syt15 | 4.72E−24 |
| PSN_1 | March1 | 7.70E−24 |
| PSN_1 | Amigo2 | 1.26E−23 |
| PSN_1 | Kcnb2 | 1.26E−23 |
| PSN_1 | Vmn2r-ps54 | 5.10E−23 |
| PSN_1 | Cysltr2 | 6.83E−23 |
| PSN_1 | Scube1 | 2.95E−22 |
| PSN_1 | Chst15 | 3.08E−22 |
| PSN_1 | Prrt2 | 3.97E−22 |
| PSN_1 | Asah2 | 4.18E−22 |
| PSN_1 | Susd2 | 4.22E−22 |
| PSN_1 | Aldh1l1 | 1.40E−21 |
| PSN_1 | Nog | 1.11E−20 |
| PSN_1 | Serpinf1 | 7.36E−19 |
| PSN_1 | Gpr126 | 1.25E−18 |
| PSN_1 | Adamts14 | 4.08E−18 |
| PSN_1 | Mybph | 8.05E−18 |
| PSN_1 | Cplx4 | 1.24E−17 |
| PSN_1 | Gm6756 | 2.69E−15 |
| PSN_1 | Gm8096 | 1.30E−14 |
| PSN_1 | Slc6a19 | 2.27E−14 |
| PSN_1 | Hey1 | 7.33E−14 |
| PSN_1 | Otof | 5.81E−13 |
| PSN_1 | Pdlim2 | 5.33E−12 |
| PSN_1 | Serpina3n | 2.92E−11 |
| PSN_1 | Gm2721 | 4.58E−11 |
| PSN_1 | Kcp | 7.59E−11 |
| PSN_1 | Arsi | 8.65E−11 |
| PSN_1 | Folhl | 1.62E−10 |
| PSN_1 | Zfp819 | 1.99E−10 |
| PSN_1 | Cox6b2 | 2.11E−09 |
| PSN_1 | Cxcr7 | 4.43E−09 |
| PSN_1 | Fmod | 5.11E−09 |
| PSN_1 | Gm16197 | 5.76E−09 |
| PSN_1 | Myh4 | 7.68E−09 |
| PSN_1 | Gstm6 | 9.74E−09 |
| PSN_1 | 4930453H23Rik | 1.64E−08 |
| PSN_1 | Tmem119 | 3.42E−08 |
| PSN_1 | E2f1 | 3.51E−08 |
| PSN_1 | Irs3 | 3.53E−08 |
| PSN_1 | Gng13 | 7.01E−08 |
| PSN_1 | Amelx | 7.20E−08 |
| PSN_1 | Gbp2 | 1.06E−07 |
| PSN_1 | Psg26 | 1.58E−07 |
| PSN_1 | Foxa2 | 1.59E−07 |
| PSN_1 | Inhbb | 9.31E−07 |
| PSN_1 | Sod3 | 9.38E−07 |
| PSN_1 | Mrap | 1.05E−06 |
| PSN_1 | Trim47 | 1.82E−06 |
| PSN_1 | 2700070H01Rik | 3.46E−06 |
| PSN_1 | Ppm1n | 4.06E−06 |
| PSN_1 | 2410124H12Rik | 4.62E−06 |
| PSN_1 | 4930417O13Rik | 4.03E−05 |
| PSN_1 | Gdf5 | 5.22E−05 |
| PSN_1 | Hrk | 9.92E−05 |
| PSN_1 | 1110032F04Rik | 1.27E−04 |
| PSN_1 | Ccdc8 | 2.72E−04 |
| PSN_1 | Gja3 | 3.65E−04 |
| PSN_1 | Oas1e | 1.20E−03 |
| PSN_1 | Chrdl2 | 3.69E−03 |
| PSN_1 | Klhl30 | 5.65E−03 |
| PSN_1 | AW011738 | 6.49E−03 |
| PSN_1 | Ppp3r2 | 2.02E−02 |
| PSN_1 | ligp1 | 2.24E−02 |
| PSN_1 | Hist1h2bp | 4.36E−02 |
| PSN_2 | Mgat4c | 6.98E−268 |
| PSN_2 | A930011G23Rik | 1.69E−247 |
| PSN_2 | Cdh9 | 6.92E−146 |
| PSN_2 | Agtr1b | 1.37E−135 |
| PSN_2 | Speer4a | 2.66E−106 |
| PSN_2 | Arhgap6 | 1.49E−89 |
| PSN_2 | Gm10471 | 4.92E−83 |
| PSN_2 | Mir466g | 3.36E−79 |
| PSN_2 | Gm10220 | 1.59E−78 |
| PSN_2 | Glra1 | 2.55E−75 |
| PSN_2 | Klhl1 | 1.51E−64 |
| PSN_2 | 5031410l06Rik | 1.03E−63 |
| PSN_2 | March1 | 7.48E−59 |
| PSN_2 | Galnt18 | 1.15E−53 |
| PSN_2 | Cdh8 | 1.21E−53 |
| PSN_2 | Serpine2 | 1.26E−53 |
| PSN_2 | Cacna2d3 | 1.06E−52 |
| PSN_2 | Vmn2r15 | 1.10E−52 |
| PSN_2 | Vwc2l | 1.35E−50 |
| PSN_2 | 9330175M20Rik | 1.42E−47 |
| PSN_2 | Ano2 | 7.32E−47 |
| PSN_2 | 2210039B01Rik | 1.30E−45 |
| PSN_2 | Tmeff2 | 2.10E−43 |
| PSN_2 | Dgkg | 2.17E−43 |
| PSN_2 | Nmur2 | 3.18E−43 |
| PSN_2 | Plcl1 | 1.11E−41 |
| PSN_2 | Sgcz | 7.60E−40 |
| PSN_2 | Gm1604b | 1.25E−38 |
| PSN_2 | Pcdh9 | 4.52E−38 |
| PSN_2 | Zbbx | 7.17E−38 |
| PSN_2 | 2610307P16Rik | 2.27E−34 |
| PSN_2 | Galnt13 | 2.80E−34 |
| PSN_2 | Cblb | 7.77E−34 |
| PSN_2 | Spock3 | 1.24E−33 |
| PSN_2 | Gm648 | 5.34E−33 |
| PSN_2 | 1700013H16Rik | 1.09E−31 |
| PSN_2 | Nek1 | 1.90E−31 |
| PSN_2 | Htr4 | 2.39E−31 |
| PSN_2 | Ctnna2 | 8.47E−31 |
| PSN_2 | Zfhx3 | 1.28E−30 |
| PSN_2 | Disp1 | 6.10E−30 |
| PSN_2 | Kif26b | 1.09E−29 |
| PSN_2 | Clstn2 | 1.17E−29 |
| PSN_2 | Sdpr | 9.03E−29 |
| PSN_2 | Mir1970 | 1.02E−27 |
| PSN_2 | Cntnap2 | 1.28E−27 |
| PSN_2 | Tcf7l2 | 1.93E−25 |
| PSN_2 | Pbx3 | 3.28E−25 |
| PSN_2 | Mapk4 | 4.62E−25 |
| PSN_2 | Kcnk2 | 9.36E−25 |
| PSN_2 | Car10 | 1.10E−24 |
| PSN_2 | Cachd1 | 1.98E−24 |
| PSN_2 | Htr1f | 2.84E−24 |
| PSN_2 | Scgn | 2.93E−24 |
| PSN_2 | Palld | 1.00E−23 |
| PSN_2 | Pax4 | 2.04E−23 |
| PSN_2 | Syt9 | 1.51E−22 |
| PSN_2 | Dgki | 4.46E−22 |
| PSN_2 | Apba1 | 4.59E−22 |
| PSN_2 | Sema5a | 5.05E−22 |
| PSN_2 | Slc2a13 | 7.60E−22 |
| PSN_2 | Robo2 | 1.96E−21 |
| PSN_2 | Ccbe1 | 2.91E−21 |
| PSN_2 | Aff3 | 3.18E−21 |
| PSN_2 | Hs6st2 | 4.29E−21 |
| PSN_2 | Cadm2 | 8.60E−21 |
| PSN_2 | Ddah1 | 3.58E−20 |
| PSN_2 | Cck | 3.96E−20 |
| PSN_2 | Speer5-ps1 | 2.70E−19 |
| PSN_2 | Ephx2 | 5.49E−19 |
| PSN_2 | Gabrg3 | 5.51E−19 |
| PSN_2 | Bcl2 | 7.73E−19 |
| PSN_2 | Clca2 | 2.63E−18 |
| PSN_2 | Nrxn3 | 1.33E−17 |
| PSN_2 | 4933416M07Rik | 1.48E−17 |
| PSN_2 | Speer7-ps1 | 8.30E−17 |
| PSN_2 | Alk | 8.76E−17 |
| PSN_2 | Epha3 | 9.02E−17 |
| PSN_2 | Rasgef1b | 9.31E−17 |
| PSN_2 | Gm20754 | 9.31E−17 |
| PSN_2 | 2410137M14Rik | 1.81E−16 |
| PSN_2 | Serpini1 | 2.79E−16 |
| PSN_2 | Osbpl6 | 5.03E−16 |
| PSN_2 | Umod | 8.62E−16 |
| PSN_2 | Vsnl1 | 1.08E−15 |
| PSN_2 | Il1rapl1 | 1.35E−15 |
| PSN_2 | Cd244 | 2.27E−15 |
| PSN_2 | Apba2 | 2.38E−15 |
| PSN_2 | Spert | 2.38E−15 |
| PSN_2 | Dlc1 | 3.02E−15 |
| PSN_2 | B3galtl | 3.36E−15 |
| PSN_2 | Tbx2 | 3.76E−15 |
| PSN_2 | Xkr4 | 4.96E−15 |
| PSN_2 | Stxbp2 | 7.18E−15 |
| PSN_2 | Ank | 7.83E−15 |
| PSN_2 | Tshz3 | 8.02E−15 |
| PSN_2 | Rph3a | 8.38E−15 |
| PSN_2 | Sntg1 | 1.24E−14 |
| PSN_2 | 1700072O05Rik | 2.23E−14 |
| PSN_2 | Pbx1 | 7.26E−14 |
| PSN_2 | Fgd2 | 1.95E−13 |
| PSN_2 | Oas2 | 2.61E−13 |
| PSN_2 | 1500009C09Rik | 2.61E−13 |
| PSN_2 | Rspo3 | 2.84E−13 |
| PSN_2 | Hormad2 | 5.28E−13 |
| PSN_2 | Gfral | 8.11E−13 |
| PSN_2 | Vmn1r58 | 1.30E−12 |
| PSN_2 | Tekt5 | 1.04E−10 |
| PSN_2 | Ucn3 | 3.24E−10 |
| PSN_2 | Gpr132 | 7.27E−10 |
| PSN_2 | Enc1 | 1.12E−09 |
| PSN_2 | Gm4745 | 5.33E−09 |
| PSN_2 | Hist1h4m | 5.70E−09 |
| PSN_2 | Fabp7 | 5.85E−09 |
| PSN_2 | Slc2a5 | 8.10E−09 |
| PSN_2 | Defb23 | 1.86E−08 |
| PSN_2 | Chrna2 | 2.11E−08 |
| PSN_2 | 4933439K11Rik | 2.30E−08 |
| PSN_2 | Slc6a13 | 2.93E−08 |
| PSN_2 | Klk1b3 | 6.91E−08 |
| PSN_2 | Lrtm1 | 1.13E−07 |
| PSN_2 | 1700017G19Rik | 1.29E−07 |
| PSN_2 | Pygo1 | 2.37E−07 |
| PSN_2 | Gstt4 | 3.77E−07 |
| PSN_2 | 1700030M09Rik | 3.84E−07 |
| PSN_2 | 6430562O15Rik | 4.16E−07 |
| PSN_2 | Dkk2 | 5.22E−07 |
| PSN_2 | Otc | 1.55E−06 |
| PSN_2 | Cpa3 | 2.98E−06 |
| PSN_2 | Dlx3 | 3.25E−06 |
| PSN_2 | Phf11a | 8.23E−06 |
| PSN_2 | 4933427l22Rik | 3.22E−05 |
| PSN_2 | Camkv | 3.49E−05 |
| PSN_2 | Fgf16 | 3.56E−05 |
| PSN_2 | Nat3 | 3.93E−05 |
| PSN_2 | Hrh1 | 4.44E−05 |
| PSN_2 | Clec2h | 5.24E−05 |
| PSN_2 | Amelx | 7.38E−05 |
| PSN_2 | Nfe2 | 1.11E−04 |
| PSN_2 | Gm14812 | 3.11E−04 |
| PSN_2 | AU023762 | 7.58E−04 |
| PSN_2 | Rnu12 | 1.38E−03 |
| PSN_2 | Mc4r | 1.74E−03 |
| PSN_2 | Emilin2 | 2.03E−03 |
| PSN_2 | Kif7 | 2.15E−03 |
| PSN_2 | Psmb11 | 2.36E−03 |
| PSN_2 | Nr2e1 | 2.97E−03 |
| PSN_2 | 4930474N09Rik | 3.02E−03 |
| PSN_2 | 5031434C07Rik | 3.16E−03 |
| PSN_2 | Sall4 | 3.59E−03 |
| PSN_2 | RpIl1 | 4.75E−03 |
| PSN_2 | Crisp3 | 4.99E−03 |
| PSN_2 | Gm1082 | 5.34E−03 |
| PSN_2 | 1700013G23Rik | 5.49E−03 |
| PSN_2 | Pabpc5 | 6.93E−03 |
| PSN_2 | 1810019D21Rik | 7.51E−03 |
| PSN_2 | Hist1h4k | 8.01E−03 |
| PSN_2 | Slc35g3 | 8.55E−03 |
| PSN_2 | Lrrc17 | 9.58E−03 |
| PSN_2 | 2610100L16Rik | 1.49E−02 |
| PSN_2 | Ccnf | 1.58E−02 |
| PSN_2 | Gm10789 | 1.78E−02 |
| PSN_2 | Al507597 | 1.97E−02 |
| PSN_2 | Nrarp | 2.28E−02 |
| PSN_2 | Ephb3 | 2.77E−02 |
| PSN_2 | 4930471C04Rik | 2.82E−02 |
| PSN_2 | 9130023H24Rik | 3.56E−02 |
| PSN_2 | Rspo1 | 3.57E−02 |
| PSN_2 | Clec4d | 4.62E−02 |
| PSN_2 | Krt16 | 4.95E−02 |
| PSN_3 | Piezo2 | 1.32E−168 |
| PSN_3 | Abca9 | 5.02E−166 |
| PSN_3 | Sema5a | 8.19E−144 |
| PSN_3 | Mir466g | 2.19E−117 |
| PSN_3 | Ror1 | 9.21E−117 |
| PSN_3 | Xcr1 | 5.07E−108 |
| PSN_3 | Gng2 | 1.03E−86 |
| PSN_3 | Sgcz | 1.78E−84 |
| PSN_3 | Kif26b | 3.50E−80 |
| PSN_3 | Kcnh7 | 5.07E−80 |
| PSN_3 | Sorbs2 | 1.17E−77 |
| PSN_3 | Syt10 | 2.02E−76 |
| PSN_3 | Ntng1 | 1.36E−69 |
| PSN_3 | Loxl3 | 9.55E−67 |
| PSN_3 | Nodal | 2.60E−57 |
| PSN_3 | Gpr126 | 3.05E−57 |
| PSN_3 | Epb4.1l3 | 3.92E−56 |
| PSN_3 | 9130019P16Rik | 5.59E−50 |
| PSN_3 | Ano5 | 2.64E−49 |
| PSN_3 | D330022K07Rik | 6.52E−49 |
| PSN_3 | Zfhx3 | 5.30E−47 |
| PSN_3 | Scgb2b2 | 4.53E−45 |
| PSN_3 | Bdnf | 1.43E−44 |
| PSN_3 | Ppara | 2.24E−44 |
| PSN_3 | Rfx6 | 5.15E−44 |
| PSN_3 | Rerg | 9.69E−44 |
| PSN_3 | BC049352 | 1.59E−43 |
| PSN_3 | Trim36 | 1.47E−42 |
| PSN_3 | Skap1 | 2.21E−40 |
| PSN_3 | Pbx3 | 2.47E−39 |
| PSN_3 | Tenm4 | 3.72E−39 |
| PSN_3 | Grid1 | 7.63E−38 |
| PSN_3 | Palm2 | 2.30E−37 |
| PSN_3 | Atp7a | 4.97E−37 |
| PSN_3 | Dapk1 | 8.51E−37 |
| PSN_3 | Mast4 | 1.56E−35 |
| PSN_3 | Spock1 | 2.94E−35 |
| PSN_3 | Rassf4 | 2.04E−33 |
| PSN_3 | Ush1c | 2.30E−33 |
| PSN_3 | Galnt18 | 3.44E−33 |
| PSN_3 | Gmpr | 6.38E−33 |
| PSN_3 | Fndc3b | 1.01E−32 |
| PSN_3 | Abca8b | 5.10E−32 |
| PSN_3 | Baiap3 | 5.46E−32 |
| PSN_3 | Tmeff2 | 8.83E−32 |
| PSN_3 | Ifi203 | 1.43E−31 |
| PSN_3 | Cck | 5.30E−31 |
| PSN_3 | Chst8 | 5.41E−31 |
| PSN_3 | Akap2 | 1.54E−30 |
| PSN_3 | Lrriq4 | 6.55E−30 |
| PSN_3 | Cd24a | 2.74E−29 |
| PSN_3 | Ddah1 | 3.35E−29 |
| PSN_3 | Rttn | 1.51E−28 |
| PSN_3 | March1 | 1.51E−28 |
| PSN_3 | Calb1 | 2.67E−28 |
| PSN_3 | Gpc6 | 2.67E−28 |
| PSN_3 | Phlda1 | 4.35E−27 |
| PSN_3 | Myo18b | 1.25E−26 |
| PSN_3 | Meis1 | 1.56E−26 |
| PSN_3 | Arhgap28 | 9.48E−26 |
| PSN_3 | Serpini1 | 9.69E−26 |
| PSN_3 | Cachd1 | 1.38E−25 |
| PSN_3 | Cpne8 | 4.27E−25 |
| PSN_3 | Calcb | 8.04E−25 |
| PSN_3 | Esyt3 | 1.04E−24 |
| PSN_3 | AW549542 | 1.42E−24 |
| PSN_3 | Dlc1 | 1.70E−24 |
| PSN_3 | Slc7a3 | 2.35E−24 |
| PSN_3 | Slc17a6 | 2.35E−24 |
| PSN_3 | Apcdd1 | 8.70E−24 |
| PSN_3 | Ccdc85a | 9.02E−24 |
| PSN_3 | Galnt14 | 3.14E−23 |
| PSN_3 | L3mbtl4 | 3.25E−23 |
| PSN_3 | Tanc2 | 3.26E−23 |
| PSN_3 | Gm5441 | 4.22E−23 |
| PSN_3 | Trps1 | 4.25E−23 |
| PSN_3 | Tnr | 8.65E−23 |
| PSN_3 | Prkca | 2.05E−22 |
| PSN_3 | Nefm | 2.41E−22 |
| PSN_3 | Nell1 | 2.48E−22 |
| PSN_3 | Nnat | 4.72E−22 |
| PSN_3 | Kazn | 1.03E−21 |
| PSN_3 | Chrm3 | 1.17E−21 |
| PSN_3 | Nefl | 2.21E−21 |
| PSN_3 | Pcp4l1 | 2.59E−21 |
| PSN_3 | Jazf1 | 3.05E−21 |
| PSN_3 | Sez6l | 8.53E−21 |
| PSN_3 | 5830418P13Rik | 1.10E−20 |
| PSN_3 | Eif3h | 2.08E−20 |
| PSN_3 | Bcl2 | 2.60E−20 |
| PSN_3 | Limch1 | 1.08E−19 |
| PSN_3 | Rxrg | 1.66E−19 |
| PSN_3 | Mndal | 1.71E−19 |
| PSN_3 | Colq | 2.24E−19 |
| PSN_3 | 2810055G20Rik | 2.92E−19 |
| PSN_3 | Bfsp2 | 3.54E−19 |
| PSN_3 | Abca8a | 3.83E−19 |
| PSN_3 | Ltk | 4.21E−19 |
| PSN_3 | Tshz3 | 4.21E−19 |
| PSN_3 | Tiam1 | 6.03E−19 |
| PSN_3 | Pdyn | 7.15E−19 |
| PSN_3 | Bmpr1b | 6.43E−18 |
| PSN_3 | Prr15 | 9.67E−18 |
| PSN_3 | Stra6 | 1.55E−17 |
| PSN_3 | Dok1 | 5.35E−16 |
| PSN_3 | Crhr2 | 1.08E−15 |
| PSN_3 | Skint1 | 3.90E−15 |
| PSN_3 | Il18r1 | 7.17E−15 |
| PSN_3 | Fbln1 | 7.64E−15 |
| PSN_3 | A330050F15Rik | 9.84E−15 |
| PSN_3 | Slc38a11 | 1.31E−14 |
| PSN_3 | Gm13278 | 1.41E−14 |
| PSN_3 | Cacng5 | 2.58E−14 |
| PSN_3 | Npy5r | 3.13E−13 |
| PSN_3 | Hmga2-ps1 | 3.27E−13 |
| PSN_3 | Bpifc | 7.29E−13 |
| PSN_3 | Nckap1l | 1.34E−12 |
| PSN_3 | Anxa1 | 2.43E−12 |
| PSN_3 | Gm5640 | 6.78E−12 |
| PSN_3 | Rem2 | 7.79E−12 |
| PSN_3 | Tas1r2 | 1.22E−11 |
| PSN_3 | Pcdh12 | 1.36E−11 |
| PSN_3 | Tmem211 | 2.28E−11 |
| PSN_3 | Zdhhc19 | 3.10E−10 |
| PSN_3 | Btnl9 | 4.70E−10 |
| PSN_3 | Gm14685 | 1.65E−09 |
| PSN_3 | Ifi204 | 2.04E−09 |
| PSN_3 | 0610007N19Rik | 1.17E−08 |
| PSN_3 | 4930452G13Rik | 2.09E−08 |
| PSN_3 | Slco1a4 | 3.98E−08 |
| PSN_3 | Mnda | 5.20E−08 |
| PSN_3 | Cd300lb | 6.35E−08 |
| PSN_3 | Ace2 | 3.05E−07 |
| PSN_3 | Cyp2g1 | 6.22E−07 |
| PSN_3 | Gprc6a | 1.32E−06 |
| PSN_3 | Eras | 1.49E−06 |
| PSN_3 | Slc15a3 | 1.84E−06 |
| PSN_3 | Fam187b | 4.69E−06 |
| PSN_3 | Gmnc | 1.05E−05 |
| PSN_3 | Gm829 | 1.10E−05 |
| PSN_3 | Il10ra | 1.41E−05 |
| PSN_3 | Olfr122 | 4.59E−05 |
| PSN_3 | Csn3 | 5.21E−05 |
| PSN_3 | Clec3a | 1.08E−04 |
| PSN_3 | Gpr26 | 1.22E−04 |
| PSN_3 | Irs3 | 2.43E−04 |
| PSN_3 | Cdhr1 | 2.70E−04 |
| PSN_3 | Lrat | 3.34E−04 |
| PSN_3 | Lrrc25 | 3.56E−04 |
| PSN_3 | C030007H22Rik | 6.55E−04 |
| PSN_3 | Kcns1 | 8.61E−04 |
| PSN_3 | Cd3g | 9.38E−04 |
| PSN_3 | Hephl1 | 1.34E−03 |
| PSN_3 | 4930461G14Rik | 1.34E−03 |
| PSN_3 | Chrna10 | 1.73E−03 |
| PSN_3 | 4933407E24Rik | 1.87E−03 |
| PSN_3 | Rbpjl | 2.16E−03 |
| PSN_3 | Elf5 | 3.96E−03 |
| PSN_3 | Vsig8 | 4.80E−03 |
| PSN_3 | Ucp1 | 5.19E−03 |
| PSN_3 | Olfr1030 | 5.76E−03 |
| PSN_3 | Iltifb | 6.10E−03 |
| PSN_3 | Fam43b | 7.39E−03 |
| PSN_3 | Vmn1r45 | 8.02E−03 |
| PSN_3 | Ldlrad2 | 1.40E−02 |
| PSN_3 | Tm4sf19 | 1.85E−02 |
| PSN_3 | 9330175E14Rik | 2.06E−02 |
| PSN_3 | Cited1 | 2.44E−02 |
| PSN_3 | Thbs2 | 2.56E−02 |
| PSN_3 | D830015G02Rik | 3.15E−02 |
| PSN_3 | G630090E17Rik | 3.48E−02 |
| PSN_3 | Gm1653 | 3.61E−02 |
| PSN_3 | Olfr59 | 3.64E−02 |
| PSN_3 | Chrng | 3.71E−02 |
| PSN_3 | Fat2 | 4.59E−02 |
| PSN_4 | Satb2 | 7.50E−224 |
| PSN_4 | 9530026P05Rik | 9.18E−167 |
| PSN_4 | Vipr2 | 3.87E−158 |
| PSN_4 | Sst | 3.90E−147 |
| PSN_4 | Chsy3 | 4.78E−142 |
| PSN_4 | Fam19a2 | 2.84E−131 |
| PSN_4 | Myrip | 6.21E−106 |
| PSN_4 | Slit3 | 1.01E−105 |
| PSN_4 | Oas1a | 8.15E−104 |
| PSN_4 | Gfra2 | 1.16E−101 |
| PSN_4 | Adamts9 | 4.12E−101 |
| PSN_4 | Gm12216 | 8.90E−100 |
| PSN_4 | Ldb2 | 3.18E−98 |
| PSN_4 | Scube1 | 1.18E−92 |
| PSN_4 | Adamts20 | 1.65E−92 |
| PSN_4 | Elmo1 | 3.32E−92 |
| PSN_4 | 2610017I09Rik | 8.39E−92 |
| PSN_4 | Plxna4 | 1.05E−91 |
| PSN_4 | Rbm20 | 2.65E−91 |
| PSN_4 | Inpp4b | 1.63E−87 |
| PSN_4 | Grp | 4.40E−80 |
| PSN_4 | Smarca2 | 4.40E−80 |
| PSN_4 | Calcb | 9.32E−80 |
| PSN_4 | Nrxn3 | 5.10E−73 |
| PSN_4 | Nell1 | 3.31E−72 |
| PSN_4 | Ccbe1 | 2.18E−71 |
| PSN_4 | Oas1g | 4.43E−71 |
| PSN_4 | Vwc2 | 1.44E−70 |
| PSN_4 | Bcl2 | 2.99E−70 |
| PSN_4 | 1810041L15Rik | 1.94E−67 |
| PSN_4 | Sel1l3 | 2.05E−67 |
| PSN_4 | Oxtr | 4.75E−67 |
| PSN_4 | Sema3c | 9.92E−67 |
| PSN_4 | Kcnn3 | 5.06E−66 |
| PSN_4 | Arhgap24 | 1.28E−65 |
| PSN_4 | Scn11a | 6.46E−65 |
| PSN_4 | St3gal6 | 1.54E−64 |
| PSN_4 | Tshz2 | 2.44E−64 |
| PSN_4 | Grm1 | 1.09E−63 |
| PSN_4 | Prrt2 | 1.48E−63 |
| PSN_4 | Dlgap2 | 7.64E−61 |
| PSN_4 | Colec12 | 8.92E−60 |
| PSN_4 | Wbscr17 | 5.40E−59 |
| PSN_4 | Rbfox1 | 5.94E−59 |
| PSN_4 | Dbc1 | 7.11E−59 |
| PSN_4 | Ptpn5 | 3.25E−57 |
| PSN_4 | Pknox2 | 4.58E−57 |
| PSN_4 | Itga6 | 4.02E−56 |
| PSN_4 | Pag1 | 1.43E−55 |
| PSN_4 | Piezo1 | 2.03E−55 |
| PSN_4 | Pcbp3 | 3.68E−55 |
| PSN_4 | Zbtb7c | 4.09E−55 |
| PSN_4 | Insc | 4.77E−55 |
| PSN_4 | Ppfibp2 | 6.05E−55 |
| PSN_4 | Frmd4b | 6.77E−55 |
| PSN_4 | Lrrn2 | 7.04E−53 |
| PSN_4 | Ptprm | 4.20E−51 |
| PSN_4 | Plod2 | 5.92E−50 |
| PSN_4 | Ptgs1 | 6.29E−50 |
| PSN_4 | Pcsk2 | 1.47E−49 |
| PSN_4 | Syn2 | 1.60E−49 |
| PSN_4 | Hnf4g | 4.50E−49 |
| PSN_4 | Pdgfd | 4.57E−49 |
| PSN_4 | Rgs9 | 4.69E−49 |
| PSN_4 | Gcgr | 1.81E−48 |
| PSN_4 | Ssbp3 | 3.33E−48 |
| PSN_4 | St6galnac3 | 4.45E−48 |
| PSN_4 | Fbxw24 | 5.53E−48 |
| PSN_4 | Ptprk | 1.07E−46 |
| PSN_4 | Dgki | 6.37E−45 |
| PSN_4 | Col5a3 | 1.27E−44 |
| PSN_4 | Begain | 5.89E−44 |
| PSN_4 | 3110047P20Rik | 8.61E−44 |
| PSN_4 | P2rx2 | 1.17E−43 |
| PSN_4 | Cachd1 | 1.29E−43 |
| PSN_4 | March4 | 2.31E−43 |
| PSN_4 | Tcf7l2 | 2.40E−43 |
| PSN_4 | Dmkn | 5.17E−43 |
| PSN_4 | Chat | 5.81E−43 |
| PSN_4 | Slc36a1 | 7.72E−43 |
| PSN_4 | Igsf3 | 8.67E−43 |
| PSN_4 | Kcnh1 | 8.67E−43 |
| PSN_4 | Dec | 9.35E−43 |
| PSN_4 | Zfp618 | 6.92E−42 |
| PSN_4 | Fbxw15 | 4.99E−41 |
| PSN_4 | Smoc2 | 4.99E−41 |
| PSN_4 | Tmcc3 | 1.07E−40 |
| PSN_4 | Pak3 | 2.84E−40 |
| PSN_4 | Dync1i1 | 5.67E−40 |
| PSN_4 | Oas1h | 8.44E−40 |
| PSN_4 | Adcy1 | 1.32E−39 |
| PSN_4 | Bnc2 | 1.41E−39 |
| PSN_4 | Casz1 | 1.93E−39 |
| PSN_4 | Ddah1 | 2.75E−39 |
| PSN_4 | Galnt5 | 3.91E−39 |
| PSN_4 | Ptprt | 3.97E−39 |
| PSN_4 | Syndig1 | 9.28E−39 |
| PSN_4 | Cdc14a | 1.27E−38 |
| PSN_4 | Adam19 | 3.86E−38 |
| PSN_4 | Nrp2 | 4.58E−38 |
| PSN_4 | Lypd6b | 8.08E−38 |
| PSN_4 | Atoh8 | 9.35E−38 |
| PSN_4 | Runx2 | 4.62E−34 |
| PSN_4 | 9130024F11Rik | 4.86E−33 |
| PSN_4 | Col24a1 | 5.10E−31 |
| PSN_4 | Crhr1 | 3.32E−29 |
| PSN_4 | Oas1d | 6.64E−29 |
| PSN_4 | Rtp4 | 3.93E−25 |
| PSN_4 | Lmcd1 | 2.54E−24 |
| PSN_4 | Nmbr | 8.24E−23 |
| PSN_4 | Olr1 | 2.21E−22 |
| PSN_4 | Sp100 | 3.97E−22 |
| PSN_4 | Cpne5 | 1.61E−21 |
| PSN_4 | Aldh1a2 | 2.38E−18 |
| PSN_4 | Robo3 | 2.06E−16 |
| PSN_4 | Palmd | 8.78E−16 |
| PSN_4 | Pinlyp | 2.29E−15 |
| PSN_4 | Cckbr | 4.29E−13 |
| PSN_4 | Tlr4 | 8.37E−13 |
| PSN_4 | Sftpc | 2.57E−11 |
| PSN_4 | Oas1e | 5.04E−11 |
| PSN_4 | Tifab | 4.07E−09 |
| PSN_4 | Th | 1.65E−08 |
| PSN_4 | 4930474M22Rik | 5.86E−08 |
| PSN_4 | Chrna6 | 2.58E−07 |
| PSN_4 | Gm10536 | 5.32E−07 |
| PSN_4 | Ermn | 3.03E−06 |
| PSN_4 | Il5 | 3.23E−06 |
| PSN_4 | Fzd6 | 4.28E−06 |
| PSN_4 | Olfr943 | 6.98E−06 |
| PSN_4 | Fbxw19 | 1.12E−05 |
| PSN_4 | Mx1 | 1.34E−05 |
| PSN_4 | Pitx3 | 1.79E−05 |
| PSN_4 | Clec7a | 1.96E−05 |
| PSN_4 | Mettl7a3 | 5.89E−05 |
| PSN_4 | Tex15 | 7.89E−05 |
| PSN_4 | Zfp503 | 8.00E−05 |
| PSN_4 | Fam84a | 3.07E−04 |
| PSN_4 | Glb1l2 | 5.50E−04 |
| PSN_4 | 1700029P11Rik | 6.24E−04 |
| PSN_4 | 1700097N02Rik | 8.15E−04 |
| PSN_4 | Gm1965 | 1.24E−03 |
| PSN_4 | Gm15348 | 1.39E−03 |
| PSN_4 | Psg-ps1 | 2.16E−03 |
| PSN_4 | 4933429O19Rik | 2.31E−03 |
| PSN_4 | Ttpa | 2.86E−03 |
| PSN_4 | Gm19990 | 3.04E−03 |
| PSN_4 | Spo11 | 3.05E−03 |
| PSN_4 | Olfr1082 | 3.23E−03 |
| PSN_4 | Acot3 | 3.41E−03 |
| PSN_4 | Dsg3 | 3.80E−03 |
| PSN_4 | Tmem8c | 3.94E−03 |
| PSN_4 | A630012P03Rik | 4.47E−03 |
| PSN_4 | Svs1 | 5.01E−03 |
| PSN_4 | Tgtp2 | 5.82E−03 |
| PSN_4 | Cxcr5 | 6.44E−03 |
| PSN_4 | Gm20556 | 6.58E−03 |
| PSN_4 | Rlbp1 | 6.84E−03 |
| PSN_4 | Olfr168 | 7.92E−03 |
| PSN_4 | Serpinb12 | 8.24E−03 |
| PSN_4 | Tex28 | 8.34E−03 |
| PSN_4 | Dio2 | 8.35E−03 |
| PSN_4 | Dmbx1 | 8.92E−03 |
| PSN_4 | Fam124b | 1.06E−02 |
| PSN_4 | Gja1 | 1.62E−02 |
| PSN_4 | Krt71 | 1.71E−02 |
| PSN_4 | Apol7a | 1.82E−02 |
| PSN_4 | Etd | 2.01E−02 |
| PSN_4 | Atp6v1e2 | 2.29E−02 |
| PSN_4 | Ankrd7 | 2.48E−02 |
| PSN_4 | Lipn | 2.78E−02 |
| PSN_4 | Padi3 | 3.39E−02 |
| PSN_4 | Snora64 | 3.59E−02 |
| PSN_4 | Gm4251 | 4.02E−02 |
| PSN_4 | Olfr166 | 4.09E−02 |
| PSN_4 | Nkx2-2 | 4.58E−02 |
| PSN_4 | Vmn2r49 | 4.88E−02 |
| PSVN_1 | Astn2 | 4.07E−179 |
| PSVN_1 | Cpne4 | 1.62E−155 |
| PSVN_1 | Adam12 | 5.19E−146 |
| PSVN_1 | Scgn | 1.95E−128 |
| PSVN_1 | Moxd1 | 4.16E−126 |
| PSVN_1 | Vip | 3.82E−90 |
| PSVN_1 | Rerg | 7.66E−87 |
| PSVN_1 | Lama4 | 1.14E−86 |
| PSVN_1 | Tcerg1l | 3.81E−80 |
| PSVN_1 | 2410114N07Rik | 2.29E−79 |
| PSVN_1 | Cpa6 | 6.36E−79 |
| PSVN_1 | Luzp2 | 1.18E−78 |
| PSVN_1 | Prex2 | 2.94E−76 |
| PSVN_1 | Tacr1 | 1.10E−75 |
| PSVN_1 | Slc6a12 | 1.93E−70 |
| PSVN_1 | Gpr149 | 1.39E−63 |
| PSVN_1 | P2rx2 | 1.25E−61 |
| PSVN_1 | 4930402F11Rik | 1.27E−55 |
| PSVN_1 | Cux2 | 2.67E−54 |
| PSVN_1 | Fst | 9.32E−51 |
| PSVN_1 | Ptpre | 1.08E−49 |
| PSVN_1 | Glp2r | 1.15E−48 |
| PSVN_1 | Slc4a4 | 8.35E−48 |
| PSVN_1 | Kcnd2 | 8.73E−48 |
| PSVN_1 | Sctr | 2.20E−45 |
| PSVN_1 | B230216N24Rik | 2.23E−45 |
| PSVN_1 | Lmo7 | 4.89E−45 |
| PSVN_1 | Col4a2 | 9.66E−45 |
| PSVN_1 | Etl4 | 1.81E−44 |
| PSVN_1 | Dbh | 4.64E−44 |
| PSVN_1 | Bai1 | 5.22E−44 |
| PSVN_1 | Tspan12 | 1.36E−43 |
| PSVN_1 | Spock3 | 2.19E−43 |
| PSVN_1 | Nav2 | 1.92E−41 |
| PSVN_1 | Arpp21 | 2.11E−41 |
| PSVN_1 | Kcnq5 | 5.15E−41 |
| PSVN_1 | Plxna4 | 2.14E−39 |
| PSVN_1 | Gpc5 | 2.17E−38 |
| PSVN_1 | Camk2a | 2.99E−38 |
| PSVN_1 | Myo16 | 2.99E−38 |
| PSVN_1 | Ebf1 | 1.50E−36 |
| PSVN_1 | Pde8b | 4.84E−36 |
| PSVN_1 | Kcnk13 | 3.70E−35 |
| PSVN_1 | Oas1g | 4.92E−35 |
| PSVN_1 | Col4a1 | 6.42E−35 |
| PSVN_1 | Grin3a | 6.62E−35 |
| PSVN_1 | Pxmp2 | 1.22E−34 |
| PSVN_1 | Bean1 | 1.56E−34 |
| PSVN_1 | Frmpd1 | 2.11E−34 |
| PSVN_1 | AW549542 | 4.01E−34 |
| PSVN_1 | Myrip | 2.69E−33 |
| PSVN_1 | Phactr1 | 2.98E−33 |
| PSVN_1 | Igfbp7 | 3.35E−33 |
| PSVN_1 | Grhl3 | 6.86E−33 |
| PSVN_1 | Cyp2c66 | 1.82E−32 |
| PSVN_1 | Npr1 | 3.60E−32 |
| PSVN_1 | 2610307P16Rik | 4.67E−32 |
| PSVN_1 | Pcdha4-g | 9.37E−32 |
| PSVN_1 | Slc22a23 | 1.02E−31 |
| PSVN_1 | 1700120G07Rik | 1.28E−31 |
| PSVN_1 | Prkd1 | 2.84E−31 |
| PSVN_1 | Wwtr1 | 3.07E−31 |
| PSVN_1 | Pappa | 4.07E−31 |
| PSVN_1 | Calb2 | 4.33E−31 |
| PSVN_1 | Lrrc55 | 1.54E−30 |
| PSVN_1 | Sacs | 3.71E−30 |
| PSVN_1 | Tmeff1 | 4.84E−30 |
| PSVN_1 | Cdh19 | 5.40E−29 |
| PSVN_1 | Mmd | 1.88E−28 |
| PSVN_1 | Ankar | 2.12E−27 |
| PSVN_1 | Gm11549 | 2.96E−27 |
| PSVN_1 | Ntng1 | 1.00E−26 |
| PSVN_1 | Nrp1 | 1.08E−26 |
| PSVN_1 | R3hdm1 | 1.46E−26 |
| PSVN_1 | Tmtc2 | 2.44E−26 |
| PSVN_1 | Acpl2 | 2.89E−26 |
| PSVN_1 | Vsig4 | 2.94E−26 |
| PSVN_1 | Orai2 | 4.26E−26 |
| PSVN_1 | Etv1 | 4.96E−26 |
| PSVN_1 | Ccdc60 | 1.07E−25 |
| PSVN_1 | Rtn4rl1 | 1.25E−25 |
| PSVN_1 | Agrn | 1.29E−25 |
| PSVN_1 | Upk3b | 4.23E−25 |
| PSVN_1 | Trim52 | 5.00E−25 |
| PSVN_1 | Kcnj5 | 7.60E−25 |
| PSVN_1 | Rmst | 1.07E−24 |
| PSVN_1 | Lphn3 | 1.53E−24 |
| PSVN_1 | Cdh6 | 1.89E−24 |
| PSVN_1 | Gm13399 | 1.98E−24 |
| PSVN_1 | Inf2 | 2.86E−24 |
| PSVN_1 | Npy2r | 3.59E−24 |
| PSVN_1 | Fbn1 | 5.78E−24 |
| PSVN_1 | Ssbp2 | 8.40E−24 |
| PSVN_1 | Prune2 | 8.81E−24 |
| PSVN_1 | 4932414N04Rik | 1.59E−23 |
| PSVN_1 | Spag17 | 1.70E−23 |
| PSVN_1 | Emr1 | 2.19E−23 |
| PSVN_1 | Tmem44 | 4.01E−23 |
| PSVN_1 | Srrm4 | 4.43E−23 |
| PSVN_1 | Cntnap2 | 4.82E−23 |
| PSVN_1 | E430016F16Rik | 1.73E−21 |
| PSVN_1 | Cbln4 | 7.18E−19 |
| PSVN_1 | Clec14a | 1.00E−17 |
| PSVN_1 | Rhox2b | 2.05E−16 |
| PSVN_1 | Tubal3 | 2.86E−11 |
| PSVN_1 | Tspan10 | 1.12E−10 |
| PSVN_1 | Bmp8a | 9.67E−10 |
| PSVN_1 | Tdrd1 | 1.47E−09 |
| PSVN_1 | Gabre | 2.04E−09 |
| PSVN_1 | Olfr1355 | 2.55E−09 |
| PSVN_1 | Gm4894 | 1.04E−08 |
| PSVN_1 | 1700061F12Rik | 8.79E−08 |
| PSVN_1 | Ssty2 | 1.06E−07 |
| PSVN_1 | 1600025M17Rik | 4.12E−07 |
| PSVN_1 | 5430403N17Rik | 4.41E−07 |
| PSVN_1 | 4931440L10Rik | 7.45E−07 |
| PSVN_1 | Slfn10-ps | 1.17E−06 |
| PSVN_1 | Ugt2b1 | 1.46E−06 |
| PSVN_1 | Klrb1b | 2.22E−06 |
| PSVN_1 | Gm15107 | 2.76E−06 |
| PSVN_1 | Htra4 | 2.92E−06 |
| PSVN_1 | Cdh5 | 3.53E−06 |
| PSVN_1 | Igfbp2 | 3.66E−06 |
| PSVN_1 | 6330407A03Rik | 5.45E−06 |
| PSVN_1 | Gm10057 | 1.01E−05 |
| PSVN_1 | Cdh22 | 1.18E−05 |
| PSVN_1 | Bpifb5 | 1.37E−05 |
| PSVN_1 | Twist2 | 2.02E−05 |
| PSVN_1 | Spic | 2.45E−05 |
| PSVN_1 | 4933406J08Rik | 2.56E−05 |
| PSVN_1 | Akap3 | 3.61E−05 |
| PSVN_1 | Tie1 | 3.73E−05 |
| PSVN_1 | Gm41 | 4.54E−05 |
| PSVN_1 | 2410003L11Rik | 5.29E−05 |
| PSVN_1 | Wfdc6a | 5.33E−05 |
| PSVN_1 | Gm10408 | 7.03E−05 |
| PSVN_1 | 1190003K10Rik | 9.39E−05 |
| PSVN_1 | Cts8-ps | 9.40E−05 |
| PSVN_1 | Crx | 1.01E−04 |
| PSVN_1 | Bst1 | 1.25E−04 |
| PSVN_1 | Edn3 | 1.33E−04 |
| PSVN_1 | Lpar3 | 1.35E−04 |
| PSVN_1 | Srd5a2 | 1.50E−04 |
| PSVN_1 | Gm20755 | 1.51E−04 |
| PSVN_1 | AW495222 | 2.00E−04 |
| PSVN_1 | 4930480M12Rik | 2.09E−04 |
| PSVN_1 | Fmo4 | 2.28E−04 |
| PSVN_1 | 1700129C05Rik | 3.17E−04 |
| PSVN_1 | Slc38a5 | 3.52E−04 |
| PSVN_1 | Vmn1r181 | 3.84E−04 |
| PSVN_1 | Siglec1 | 4.01E−04 |
| PSVN_1 | Olfr1167 | 4.13E−04 |
| PSVN_1 | Wfdc11 | 4.83E−04 |
| PSVN_1 | Gsdma2 | 6.78E−04 |
| PSVN_1 | Plin1 | 8.08E−04 |
| PSVN_1 | Ugt2a1 | 8.62E−04 |
| PSVN_1 | Ncf4 | 1.50E−03 |
| PSVN_1 | Rab19 | 1.57E−03 |
| PSVN_1 | Vmn1r132 | 1.58E−03 |
| PSVN_1 | Bin2 | 1.84E−03 |
| PSVN_1 | Ercc6l | 1.88E−03 |
| PSVN_1 | Gm14781 | 1.89E−03 |
| PSVN_1 | E130006D01Rik | 1.98E−03 |
| PSVN_1 | Ermap | 2.04E−03 |
| PSVN_1 | 4930547C10Rik | 2.10E−03 |
| PSVN_1 | Gm20815 | 2.10E−03 |
| PSVN_1 | Pax3 | 2.32E−03 |
| PSVN_1 | Hist1h4c | 2.41E−03 |
| PSVN_1 | Gm20917 | 2.42E−03 |
| PSVN_1 | Cst13 | 3.04E−03 |
| PSVN_1 | Gm7714 | 3.41E−03 |
| PSVN_1 | Tsga13 | 3.50E−03 |
| PSVN_1 | Smgc | 4.12E−03 |
| PSVN_1 | Olfr869 | 5.65E−03 |
| PSVN_1 | Ticrr | 5.72E−03 |
| PSVN_1 | Msln1 | 6.19E−03 |
| PSVN_1 | Ankrd1 | 6.64E−03 |
| PSVN_1 | Serpinb7 | 6.66E−03 |
| PSVN_1 | Gm10413 | 8.97E−03 |
| PSVN_1 | Otop1 | 1.04E−02 |
| PSVN_1 | 1190003J15Rik | 1.16E−02 |
| PSVN_1 | Bpifa6 | 1.33E−02 |
| PSVN_1 | Slc6a7 | 1.55E−02 |
| PSVN_1 | Dusp27 | 1.71E−02 |
| PSVN_1 | Scnn1g | 1.79E−02 |
| PSVN_1 | Cd8b1 | 1.88E−02 |
| PSVN_1 | Rhox3h | 3.44E−02 |
| PSVN_1 | 5430421F17Rik | 4.40E−02 |
| PSVN_1 | Scn4b | 4.86E−02 |
| PSVN_2 | Trhde | 1.92E−285 |
| PSVN_2 | Col18a1 | 1.10E−160 |
| PSVN_2 | Mctp1 | 1.65E−151 |
| PSVN_2 | Gal | 8.04E−141 |
| PSVN_2 | Myo1e | 2.24E−96 |
| PSVN_2 | Ebf1 | 7.52E−92 |
| PSVN_2 | Greb11 | 1.27E−91 |
| PSVN_2 | Cntn4 | 3.63E−85 |
| PSVN_2 | St18 | 3.45E−84 |
| PSVN_2 | Al593442 | 3.37E−83 |
| PSVN_2 | Cdh10 | 9.66E−81 |
| PSVN_2 | Mical2 | 2.25E−76 |
| PSVN_2 | Efemp1 | 2.36E−75 |
| PSVN_2 | Col19a1 | 3.75E−72 |
| PSVN_2 | Rmst | 5.89E−72 |
| PSVN_2 | Myo16 | 7.52E−72 |
| PSVN_2 | Lphn2 | 1.42E−70 |
| PSVN_2 | Glp2r | 4.58E−67 |
| PSVN_2 | Man1c1 | 1.42E−66 |
| PSVN_2 | Cpa6 | 1.95E−66 |
| PSVN_2 | Neurod6 | 9.01E−66 |
| PSVN_2 | Gad2 | 2.82E−63 |
| PSVN_2 | Gm8179 | 2.58E−61 |
| PSVN_2 | Plekhg3 | 9.29E−61 |
| PSVN_2 | Cntnap2 | 1.63E−58 |
| PSVN_2 | Tmc3 | 4.12E−58 |
| PSVN_2 | Prkd1 | 1.45E−56 |
| PSVN_2 | Fbn2 | 9.62E−56 |
| PSVN_2 | Kcnk13 | 1.28E−55 |
| PSVN_2 | Kcnd2 | 5.85E−54 |
| PSVN_2 | Egfem1 | 1.17E−53 |
| PSVN_2 | Gm1715 | 8.71E−53 |
| PSVN_2 | Fstl4 | 6.20E−51 |
| PSVN_2 | BC051070 | 7.60E−51 |
| PSVN_2 | Cacna1i | 8.24E−50 |
| PSVN_2 | Ccser1 | 1.12E−49 |
| PSVN_2 | Rasgrf2 | 1.48E−49 |
| PSVN_2 | Col4a2 | 3.50E−48 |
| PSVN_2 | Trps1 | 7.09E−46 |
| PSVN_2 | Mpp4 | 6.29E−45 |
| PSVN_2 | Glyctk | 1.28E−42 |
| PSVN_2 | 2010016l18Rik | 3.15E−42 |
| PSVN_2 | Zfp385b | 1.43E−41 |
| PSVN_2 | Arpp21 | 1.69E−41 |
| PSVN_2 | Fgf12 | 3.51E−41 |
| PSVN_2 | Csf2rb2 | 5.07E−41 |
| PSVN_2 | Ccdc85a | 8.27E−41 |
| PSVN_2 | Sdk1 | 4.20E−40 |
| PSVN_2 | Asb2 | 1.42E−39 |
| PSVN_2 | Etv1 | 1.23E−38 |
| PSVN_2 | Prkg2 | 8.16E−38 |
| PSVN_2 | Dnahc9 | 1.49E−37 |
| PSVN_2 | Eml6 | 5.60E−37 |
| PSVN_2 | Hs6st3 | 1.15E−36 |
| PSVN_2 | Asic2 | 2.61E−36 |
| PSVN_2 | Sctr | 2.37E−35 |
| PSVN_2 | Als2 | 1.47E−34 |
| PSVN_2 | Trpm3 | 7.22E−34 |
| PSVN_2 | Kcnk10 | 1.01E−33 |
| PSVN_2 | Snca | 7.15E−33 |
| PSVN_2 | Col26a1 | 9.60E−33 |
| PSVN_2 | Tll2 | 1.02E−32 |
| PSVN_2 | Slc18a2 | 1.51E−32 |
| PSVN_2 | Ece1 | 5.02E−32 |
| PSVN_2 | Fmn1 | 1.22E−31 |
| PSVN_2 | Rtn4r11 | 1.60E−31 |
| PSVN_2 | Cdh1l | 3.63E−30 |
| PSVN_2 | Tll1 | 5.39E−29 |
| PSVN_2 | Camkk2 | 7.71E−29 |
| PSVN_2 | Mbnl1 | 9.73E−29 |
| PSVN_2 | Pid1 | 3.77E−28 |
| PSVN_2 | 5530401A14Rik | 3.83E−28 |
| PSVN_2 | Gfra1 | 3.83E−28 |
| PSVN_2 | Dhrs7c | 4.98E−28 |
| PSVN_2 | Ltk | 7.42E−28 |
| PSVN_2 | Agfg1 | 1.13E−27 |
| PSVN_2 | Stard5 | 1.35E−27 |
| PSVN_2 | Schip1 | 6.42E−27 |
| PSVN_2 | Mgat4a | 6.80E−27 |
| PSVN_2 | Gabrb3 | 2.24E−26 |
| PSVN_2 | Map3k5 | 3.15E−26 |
| PSVN_2 | Csf2rb | 5.64E−26 |
| PSVN_2 | Shisa6 | 1.75E−25 |
| PSVN_2 | Baalc | 3.09E−25 |
| PSVN_2 | Nedd4l | 6.12E−25 |
| PSVN_2 | 3110047P20Rik | 6.32E−25 |
| PSVN_2 | Frem1 | 1.08E−24 |
| PSVN_2 | Myt1l | 1.27E−24 |
| PSVN_2 | Npas3 | 1.42E−24 |
| PSVN_2 | Nrip3 | 2.56E−24 |
| PSVN_2 | Cd209c | 4.14E−24 |
| PSVN_2 | Smtnl2 | 4.51E−24 |
| PSVN_2 | Mrc2 | 4.59E−24 |
| PSVN_2 | Tmem232 | 7.10E−24 |
| PSVN_2 | Oxr1 | 8.35E−24 |
| PSVN_2 | Ttc39b | 9.71E−24 |
| PSVN_2 | Scgn | 1.17E−23 |
| PSVN_2 | Enox1 | 1.50E−23 |
| PSVN_2 | Kcnj5 | 1.78E−23 |
| PSVN_2 | March11 | 2.19E−23 |
| PSVN_2 | Trpv6 | 5.49E−21 |
| PSVN_2 | Abcc12 | 3.92E−20 |
| PSVN_2 | D430036J16Rik | 2.14E−17 |
| PSVN_2 | Umodl1 | 9.76E−16 |
| PSVN_2 | Gm12159 | 1.58E−15 |
| PSVN_2 | Fam131c | 9.58E−15 |
| PSVN_2 | Best3 | 2.84E−14 |
| PSVN_2 | Ms4a2 | 9.34E−13 |
| PSVN_2 | Tpbg | 1.08E−12 |
| PSVN_2 | Htr1b | 1.96E−12 |
| PSVN_2 | 4933430N04Rik | 6.73E−12 |
| PSVN_2 | Scimp | 1.24E−11 |
| PSVN_2 | Fam159b | 1.70E−11 |
| PSVN_2 | Gm13124 | 1.38E−10 |
| PSVN_2 | Psat1 | 1.05E−09 |
| PSVN_2 | Ascl3 | 2.43E−09 |
| PSVN_2 | Magel2 | 1.14E−08 |
| PSVN_2 | Vmn2r86 | 1.43E−08 |
| PSVN_2 | Gm3279 | 1.60E−08 |
| PSVN_2 | Il2rb | 1.04E−07 |
| PSVN_2 | Inhba | 1.15E−07 |
| PSVN_2 | Tex35 | 2.38E−07 |
| PSVN_2 | Grin2c | 7.46E−07 |
| PSVN_2 | Epor | 7.52E−07 |
| PSVN_2 | Tslp | 8.18E−07 |
| PSVN_2 | Opalin | 1.47E−06 |
| PSVN_2 | Spaca3 | 5.27E−06 |
| PSVN_2 | Gm1045 | 6.18E−06 |
| PSVN_2 | Ces2f | 3.05E−05 |
| PSVN_2 | Micalcl | 3.24E−05 |
| PSVN_2 | BB557941 | 3.52E−05 |
| PSVN_2 | Cuzd1 | 3.84E−05 |
| PSVN_2 | Col6a5 | 5.96E−05 |
| PSVN_2 | Pde6c | 7.94E−05 |
| PSVN_2 | Onecut1 | 1.01E−04 |
| PSVN_2 | Ly6g6d | 1.05E−04 |
| PSVN_2 | 4930453L07Rik | 1.57E−04 |
| PSVN_2 | Gm13944 | 2.62E−04 |
| PSVN_2 | Wnt3 | 3.03E−04 |
| PSVN_2 | Inmt | 3.29E−04 |
| PSVN_2 | Cthrc1 | 4.56E−04 |
| PSVN_2 | Olfr691 | 6.08E−04 |
| PSVN_2 | 4933402J07Rik | 6.70E−04 |
| PSVN_2 | Olfr301 | 8.44E−04 |
| PSVN_2 | S100a9 | 1.34E−03 |
| PSVN_2 | Rgs9bp | 1.39E−03 |
| PSVN_2 | 4930524C18Rik | 1.83E−03 |
| PSVN_2 | Pdlim4 | 2.52E−03 |
| PSVN_2 | Gm6537 | 3.57E−03 |
| PSVN_2 | Smok2a | 6.58E−03 |
| PSVN_2 | Il12b | 7.85E−03 |
| PSVN_2 | Tuba3a | 8.51E−03 |
| PSVN_2 | Cecr6 | 8.86E−03 |
| PSVN_2 | Icam1 | 9.34E−03 |
| PSVN_2 | Fcrlb | 9.40E−03 |
| PSVN_2 | 2310001K24Rik | 1.05E−02 |
| PSVN_2 | Kcnk7 | 1.22E−02 |
| PSVN_2 | F11 | 1.24E−02 |
| PSVN_2 | D730048106Rik | 1.99E−02 |
| PSVN_2 | Cacng1 | 2.07E−02 |
| PSVN_2 | Gm11756 | 2.11E−02 |
| PSVN_2 | AU022793 | 2.12E−02 |
| PSVN_2 | H1fnt | 2.26E−02 |
| PSVN_2 | Hapln2 | 2.28E−02 |
| PSVN_2 | 1700056E22Rik | 2.31E−02 |
| PSVN_2 | Piwil1 | 2.45E−02 |
| PSVN_2 | Gm4814 | 2.55E−02 |
| PSVN_2 | Klra3 | 3.86E−02 |
| PSVN_2 | Nrl | 4.49E−02 |
| PSVN_2 | Gm7538 | 4.68E−02 |

TABLE 15

| ident | gene | padjH |
|---|---|---|
| ageOld | Srsf2 | 6.07766E−16 |
| ageOld | Car1 | 6.09443E−15 |
| ageOld | Tmem181c-ps | 7.91498E−14 |
| ageOld | Spag5 | 3.67891E−09 |
| ageOld | Fgf14 | 6.87261E−09 |
| ageOld | Actb | 1.23307E−08 |
| ageOld | Mptx1 | 1.40342E−07 |
| ageOld | Grid1 | 8.60015E−07 |
| ageOld | Zg16 | 3.74902E−06 |
| ageOld | Fth1 | 5.21996E−06 |
| ageOld | Rps23 | 2.76709E−05 |
| ageOld | Park2 | 4.3162E−05 |
| ageOld | Klf8 | 6.86159E−05 |
| ageOld | Fcrla | 6.86159E−05 |
| ageOld | 1810065E05Rik | 8.77811E−05 |
| ageOld | Gm15319 | 9.52802E−05 |
| ageOld | Ildr2 | 0.000267131 |
| ageOld | Lgals1 | 0.000392336 |
| ageOld | Cyp2c55 | 0.000426978 |
| ageOld | Ly6h | 0.000481607 |
| ageOld | S100a6 | 0.000571241 |
| ageOld | Gpr158 | 0.000571241 |
| ageOld | Al317395 | 0.000695207 |
| ageOld | Katnbl1 | 0.000862634 |
| ageOld | Frem3 | 0.000862634 |
| ageOld | Kcnk12 | 0.000862634 |
| ageOld | Lin7c | 0.000862634 |
| ageOld | Sycn | 0.000993206 |
| ageOld | 1500032L24Rik | 0.001146544 |
| ageOld | Tuba1c | 0.001146743 |
| ageOld | A730008H23Rik | 0.001199386 |
| ageOld | Xrra1 | 0.001249032 |
| ageOld | Kcnd2 | 0.001249032 |
| ageOld | Cox4i1 | 0.002001917 |
| ageOld | Clca3 | 0.002209389 |
| ageOld | Gm13247 | 0.002209389 |
| ageOld | Rasl2-9 | 0.002209389 |
| ageOld | Syt2 | 0.002454158 |
| ageOld | Glt1d1 | 0.002547402 |
| ageOld | A330023F24Rik | 0.003040926 |
| ageOld | Frmd4a | 0.003472805 |
| ageOld | Man1c1 | 0.003555611 |
| ageOld | Insm2 | 0.003555611 |
| ageOld | Apbb1 | 0.003555611 |
| ageOld | Gm4832 | 0.003583683 |
| ageOld | Lnx2 | 0.003921359 |
| ageOld | Prph | 0.003921359 |
| ageOld | Degs1 | 0.003921359 |
| ageOld | Lrrk2 | 0.003944509 |
| ageOld | Mtmr14 | 0.004301636 |
| ageOld | Csmd3 | 0.004769697 |
| ageOld | Gm14525 | 0.004871094 |
| ageOld | Kif13b | 0.004892722 |
| ageOld | Rgs9 | 0.005087452 |
| ageOld | Gm6548 | 0.005087452 |
| ageOld | Kcnq3 | 0.005087452 |
| ageOld | Cst3 | 0.005087452 |
| ageOld | Rpl14 | 0.005103391 |
| ageOld | Agbl4 | 0.005343339 |
| ageOld | Dok6 | 0.005458881 |
| ageOld | 1810034E14Rik | 0.005511478 |
| ageOld | Gm13710 | 0.005511478 |
| ageOld | BC147527 | 0.005511478 |
| ageOld | DQ267100 | 0.005687892 |
| ageOld | Rps20 | 0.005725902 |
| ageOld | Cdyl | 0.006028623 |
| ageOld | Ubb | 0.006028623 |
| ageOld | Gm15421 | 0.006028623 |
| ageOld | Slc25a41 | 0.006140714 |
| ageOld | 1700030F04Rik | 0.00671004 |
| ageOld | Coa3 | 0.006828839 |
| ageOld | Nop10 | 0.006901915 |
| ageOld | Gm6682 | 0.006916998 |
| ageOld | E330020D12Rik | 0.006980499 |
| ageOld | Atp6v0b | 0.006980499 |
| ageOld | Tmem132c | 0.006980499 |
| ageOld | Gadd45gip1 | 0.006980499 |
| ageOld | Galnt13 | 0.006980499 |
| ageOld | Mdh2 | 0.007068543 |
| ageOld | Grb10 | 0.007941134 |
| ageOld | Fxyd1 | 0.007941134 |
| ageOld | Znrf3 | 0.007941134 |
| ageOld | B3gnt5 | 0.007941134 |
| ageOld | Ppia | 0.007941134 |
| ageOld | Pdzrn4 | 0.007941134 |
| ageOld | Maml3 | 0.007941134 |
| ageOld | Serpine2 | 0.008006634 |
| ageOld | Rnasek | 0.008063272 |
| ageOld | Ntrk2 | 0.008172838 |
| ageOld | Lrrc32 | 0.008504862 |
| ageOld | Rpl4 | 0.008504862 |
| ageOld | Hist3h2ba | 0.008833056 |
| ageOld | Dapk1 | 0.009165737 |
| ageOld | Zfp708 | 0.00923333 |
| ageOld | Stk40 | 0.009585982 |
| ageOld | H2afz | 0.009807021 |
| ageOld | Nudc | 0.009841722 |
| ageOld | Slc25a5 | 0.010310706 |
| ageOld | Osmr | 0.010338349 |
| ageOld | 1700126H18Rik | 0.010338349 |
| ageOld | Hsd3b5 | 0.010651553 |
| ageOld | Hist1h2bb | 0.011822191 |
| ageOld | Sprr2a1 | 0.011864799 |
| ageOld | Mkx | 0.01197707 |
| ageOld | Cables1 | 0.013906124 |
| ageOld | Rps27 | 0.01393806 |
| ageOld | Sprr2a2 | 0.01393806 |
| ageOld | Sox5 | 0.01393806 |
| ageOld | Snw1 | 0.01393806 |
| ageOld | Gm20750 | 0.014721298 |
| ageOld | Nsun3 | 0.015015236 |
| ageOld | A330040F15Rik | 0.015161855 |
| ageOld | Fbln7 | 0.015161855 |
| ageOld | Gm9758 | 0.015161855 |
| ageOld | Praf2 | 0.015271991 |
| ageOld | Slc12a5 | 0.015650837 |
| ageOld | Pfn1 | 0.01664306 |
| ageOld | Eras | 0.018128683 |
| ageOld | Snora34 | 0.018179864 |
| ageOld | Eif3c | 0.018179864 |
| ageOld | Fkrp | 0.020587043 |
| ageOld | Akr1c19 | 0.020587043 |
| ageOld | Rnf128 | 0.021128227 |
| ageOld | Lmx1b | 0.02123735 |
| ageOld | Nell2 | 0.02123735 |
| ageOld | Ednrb | 0.022763491 |
| ageOld | Gm17019 | 0.024302519 |
| ageOld | Wnt5b | 0.025124717 |
| ageOld | Rhox1 | 0.025753926 |
| ageOld | Emcn | 0.026396683 |
| ageOld | Sema3b | 0.026532981 |
| ageOld | Il15ra | 0.026690774 |
| ageOld | Gm853 | 0.026901659 |
| ageOld | Tff3 | 0.026901659 |
| ageOld | Hoxc4 | 0.026982377 |
| ageOld | Sdr42e1 | 0.027257207 |
| ageOld | Zfp259 | 0.029003604 |
| ageOld | Otog | 0.029492315 |
| ageOld | Gm4907 | 0.03012438 |
| ageOld | Lgals12 | 0.030489031 |
| ageOld | Slc8a3 | 0.031611422 |
| ageOld | Kctd2 | 0.032259761 |
| ageOld | 4833420G17Rik | 0.032259761 |
| ageOld | Kcnk1 | 0.032907243 |
| ageOld | Nav2 | 0.03465402 |
| ageOld | Tubb6 | 0.035965408 |
| ageOld | Ccdc152 | 0.036207742 |
| ageOld | Tubb2a-ps2 | 0.036657963 |
| ageOld | Bckdha | 0.036704894 |
| ageOld | Cmtm3 | 0.040041837 |
| ageOld | Yipf4 | 0.040041837 |
| ageOld | Dgat2 | 0.041204157 |
| ageOld | 1700084F23Rik | 0.043156316 |
| ageOld | Gm101 | 0.044744139 |
| ageOld | Ampd1 | 0.044744139 |
| ageOld | Tnfsf10 | 0.045051174 |
| ageOld | Slc18a3 | 0.045478161 |
| ageOld | Tmem79 | 0.045478161 |
| ageOld | Tubg1 | 0.046083911 |
| ageOld | Atp5g1 | 0.047100837 |
| ageOld | Mill2 | 0.047682204 |
| ageOld | Zfp595 | 0.048095699 |
| ageOld | Alyref2 | 0.048476918 |
| ageOld | Csl | 0.049020179 |
| ageOld | Rapsn | 0.049033248 |
| ageOld | Serpinb9c | 0.049033248 |
| ageOld | Gm766 | 0.049708376 |
| creUchl1 | Gm13710 | 4.2735E−223 |
| creUchl1 | Slc15a2 | 5.1994E−130 |
| creUchl1 | Klk1b22 | 1.6295E−127 |
| creUchl1 | Pisd-ps3 | 1.2742E−112 |
| creUchl1 | Sft2d2 | 3.0183E−99 |
| creUchl1 | Retnlb | 3.01731E−97 |
| creUchl1 | Dlgap1 | 5.39245E−94 |
| creUchl1 | Ccrn4l | 1.37044E−92 |
| creUchl1 | Ulk4 | 1.95177E−85 |
| creUchl1 | Dpp10 | 8.96092E−85 |
| creUchl1 | Gm3893 | 5.96367E−75 |
| creUchl1 | Celf3 | 1.43761E−73 |
| creUchl1 | Kcnh6 | 6.37973E−72 |
| creUchl1 | Gm8909 | 6.49835E−72 |
| creUchl1 | H2-Q9 | 1.15933E−68 |
| creUchl1 | H2-Q5 | 2.60328E−67 |
| creUchl1 | Mill2 | 4.08829E−66 |
| creUchl1 | Rbm5 | 2.96937E−64 |
| creUchl1 | Lrrfip1 | 7.17414E−63 |
| creUchl1 | Klk1b24 | 6.19282E−62 |
| creUchl1 | Klk1b21 | 1.70466E−59 |
| creUchl1 | Ybx1 | 5.68308E−59 |
| creUchl1 | Picalm | 1.90543E−56 |
| creUchl1 | Srsf2 | 4.88531E−56 |
| creUchl1 | H2-Q4 | 3.73991E−54 |
| creUchl1 | Muc2 | 8.22203E−53 |
| creUchl1 | Csad | 6.59049E−49 |
| creUchl1 | Park2 | 3.72467E−48 |
| creUchl1 | Ccl27a | 6.34247E−48 |
| creUchl1 | Agbl4 | 1.99264E−47 |
| creUchl1 | Spag5 | 9.10051E−47 |
| creUchl1 | 4933409K07Rik | 6.95104E−46 |
| creUchl1 | Zfp69 | 1.34956E−45 |
| creUchl1 | Phgr1 | 1.93335E−45 |
| creUchl1 | Cnksr2 | 5.44495E−43 |
| creUchl1 | Lypd8 | 9.90467E−43 |
| creUchl1 | H2-K2 | 1.98848E−40 |
| creUchl1 | Alcam | 7.94138E−40 |
| creUchl1 | H2-Q1 | 1.92544E−39 |
| creUchl1 | Sidt1 | 1.92544E−39 |
| creUchl1 | Pla2g2a | 5.47989E−39 |
| creUchl1 | Guca2a | 1.18948E−38 |
| creUchl1 | BC117090 | 6.15387E−38 |
| creUchl1 | Rims1 | 6.15387E−38 |
| creUchl1 | Mdga2 | 2.51402E−37 |
| creUchl1 | Rftn1 | 1.88305E−36 |
| creUchl1 | Gal | 1.52923E−35 |
| creUchl1 | Miip | 1.95768E−35 |
| creUchl1 | Akap6 | 3.26377E−35 |
| creUchl1 | F2rl2 | 1.29829E−34 |
| creUchl1 | Col5a2 | 2.88333E−34 |
| creUchl1 | Hist1h2bb | 3.12393E−34 |
| creUchl1 | A530054K11Rik | 2.76122E−33 |
| creUchl1 | Parp3 | 8.41797E−33 |
| creUchl1 | Hist1h2bf | 5.36091E−32 |
| creUchl1 | Cd163l1 | 7.19204E−32 |
| creUchl1 | Lgals4 | 9.38785E−32 |
| creUchl1 | Glt1d1 | 1.37936E−31 |
| creUchl1 | 3110007F17Rik | 2.17533E−31 |
| creUchl1 | Hspa8 | 3.75661E−31 |
| creUchl1 | Klk1b27 | 4.94029E−31 |
| creUchl1 | Gcnt4 | 5.6452E−31 |
| creUchl1 | Acaa1b | 5.6771E−31 |
| creUchl1 | H2-M5 | 6.60765E−31 |
| creUchl1 | Hist1h2bm | 1.63291E−30 |
| creUchl1 | H2-Bl | 1.718E−30 |
| creUchl1 | Ptchd4 | 1.8279E−30 |
| creUchl1 | Map6 | 3.45255E−30 |
| creUchl1 | Lin7c | 3.56589E−30 |
| creUchl1 | Ush1c | 8.591E−30 |
| creUchl1 | Clvs2 | 1.16157E−29 |
| creUchl1 | Cnep1r1 | 2.85064E−29 |
| creUchl1 | Ceacam1 | 7.19479E−29 |
| creUchl1 | Cntn5 | 1.17301E−28 |
| creUchl1 | Sgcz | 2.63549E−28 |
| creUchl1 | 1700016L04Rik | 3.07577E−28 |
| creUchl1 | H2-T24 | 8.04211E−28 |
| creUchl1 | Pcdh9 | 9.50962E−28 |
| creUchl1 | Lhfp | 2.1352E−27 |
| creUchl1 | Zfp804a | 3.77034E−27 |
| creUchl1 | Alad | 4.2361E−27 |
| creUchl1 | Prdx6b | 4.2361E−27 |
| creUchl1 | Rnf121 | 4.63125E−27 |
| creUchl1 | Gm10125 | 5.50223E−27 |
| creUchl1 | Nlrp5-ps | 8.85582E−27 |
| creUchl1 | Ccdc85a | 1.21731E−26 |
| creUchl1 | Dcc | 3.93053E−26 |
| creUchl1 | B2m | 5.38627E−26 |
| creUchl1 | 2610035D17Rik | 6.32541E−26 |
| creUchl1 | Gpc6 | 1.65307E−25 |
| creUchl1 | P2ry6 | 1.9365E−25 |
| creUchl1 | Gm13305 | 8.60062E−25 |
| creUchl1 | Magohb | 1.13325E−24 |
| creUchl1 | Ppfia2 | 1.15746E−24 |
| creUchl1 | Eda | 1.29817E−24 |
| creUchl1 | Slc25a5 | 1.64471E−24 |
| creUchl1 | Hp1bp3 | 3.97608E−24 |
| creUchl1 | Tpt1 | 4.25055E−24 |
| creUchl1 | Zfp933 | 5.78568E−24 |
| creUchl1 | Lrfn5 | 6.1858E−24 |
| creUchl1 | Hist2h2bb | 2.80492E−23 |
| creUchl1 | Klk1b11 | 3.36773E−23 |
| creUchl1 | Aif1 | 4.46261E−21 |
| creUchl1 | Glp1r | 1.42772E−18 |
| creUchl1 | Slpi | 3.73363E−17 |
| creUchl1 | Fbln7 | 1.4645E−16 |
| creUchl1 | Ror2 | 5.30774E−14 |
| creUchl1 | AA388235 | 8.08526E−13 |
| creUchl1 | Aldh1a1 | 3.92692E−12 |
| creUchl1 | 2210039B01Rik | 9.15718E−12 |
| creUchl1 | Btnl4 | 3.72023E−10 |
| creUchl1 | Lyz1 | 7.70189E−10 |
| creUchl1 | Gm853 | 1.99949E−09 |
| creUchl1 | Scrg1 | 1.20341E−08 |
| creUchl1 | Klk1b1 | 4.69649E−08 |
| creUchl1 | Trim43b | 3.09257E−07 |
| creUchl1 | Gm11194 | 3.58963E−07 |
| creUchl1 | Dcdc2a | 8.54491E−07 |
| creUchl1 | Beta-s | 1.19204E−06 |
| creUchl1 | Abcc3 | 1.42355E−06 |
| creUchl1 | Pyroxd2 | 3.3952E−06 |
| creUchl1 | Slc39a4 | 5.44387E−06 |
| creUchl1 | Hist1h2ba | 1.31435E−05 |
| creUchl1 | Cd177 | 1.95339E−05 |
| creUchl1 | Npc1l1 | 4.44712E−05 |
| creUchl1 | Gbp1 | 4.65027E−05 |
| creUchl1 | Kif20b | 5.97114E−05 |
| creUchl1 | Hal | 6.00534E−05 |
| creUchl1 | 1700109F18Rik | 8.16682E−05 |
| creUchl1 | Prss41 | 8.38497E−05 |
| creUchl1 | 4930402F11Rik | 0.000294652 |
| creUchl1 | Slc51a | 0.000379863 |
| creUchl1 | 9130008F23Rik | 0.000577028 |
| creUchl1 | Thpo | 0.000690239 |
| creUchl1 | 1810019J16Rik | 0.000717694 |
| creUchl1 | Gpr139 | 0.000740082 |
| creUchl1 | Il10ra | 0.000914087 |
| creUchl1 | Fcgr1 | 0.000974108 |
| creUchl1 | Spin4 | 0.001144009 |
| creUchl1 | Cd5 | 0.001217532 |
| creUchl1 | Awat2 | 0.001632976 |
| creUchl1 | Gja3 | 0.001687002 |
| creUchl1 | 1700040N02Rik | 0.001715414 |
| creUchl1 | Rfx6 | 0.001759615 |
| creUchl1 | Dgat2l6 | 0.001879127 |
| creUchl1 | Dnajb13 | 0.00204671 |
| creUchl1 | Rhou | 0.002096123 |
| creUchl1 | Sult6b1 | 0.002431541 |
| creUchl1 | Slamf1 | 0.004178791 |
| creUchl1 | Gm6936 | 0.005040296 |
| creUchl1 | Cd40 | 0.005835231 |
| creUchl1 | Ube2t | 0.006097185 |
| creUchl1 | Stag3 | 0.006245129 |
| creUchl1 | Lefty2 | 0.006485665 |
| creUchl1 | Mfge8 | 0.00652646 |
| creUchl1 | E330012B07Rik | 0.007024439 |
| creUchl1 | Cd14 | 0.007254097 |
| creUchl1 | Eras | 0.010898441 |
| creUchl1 | Khdc3 | 0.013260293 |
| creUchl1 | Cbs | 0.014165671 |
| creUchl1 | Ldlrad2 | 0.017800706 |
| creUchl1 | Rhox2h | 0.019220788 |
| creUchl1 | Vmn2r6 | 0.020214484 |
| creUchl1 | Tmem119 | 0.020600584 |
| creUchl1 | Sod3 | 0.022987632 |
| creUchl1 | Gm3716 | 0.023731509 |
| creUchl1 | Mtl5 | 0.028100377 |
| creUchl1 | Sigirr | 0.030921788 |
| creUchl1 | Plg | 0.032516554 |
| creUchl1 | Mir338 | 0.0325854 |
| creUchl1 | 4930433l11Rik | 0.033794892 |
| creUchl1 | Olfr287 | 0.035394182 |
| creUchl1 | Plekhg6 | 0.036120445 |
| creUchl1 | E030044B06Rik | 0.037433981 |
| creUchl1 | Bin2 | 0.043606602 |
| creUchl1 | Tdrd1 | 0.045254704 |
| creUchl1 | Igsf23 | 0.046615514 |
| creUchl1 | 1700034G24Rik | 0.047071049 |
| creUchl1 | Tal1 | 0.04848236 |
| creWNT1 | Allc | 2.46861E−13 |
| creWNT1 | B4galnt3 | 6.68121E−08 |
| creWNT1 | Cylc1 | 6.68121E−08 |
| creWNT1 | C730002L08Rik | 2.58796E−07 |
| creWNT1 | Gm3696 | 3.21851E−06 |
| creWNT1 | Serpinb9c | 3.79883E−06 |
| creWNT1 | Cd70 | 7.39893E−06 |
| creWNT1 | Car5a | 2.21711E−05 |
| creWNT1 | Prss12 | 2.27632E−05 |
| creWNT1 | Vmn2r81 | 2.29544E−05 |
| creWNT1 | Ptprq | 3.39912E−05 |
| creWNT1 | Rftn1 | 0.000114291 |
| creWNT1 | Mir669a-7 | 0.000374422 |
| creWNT1 | Fam78a | 0.000498802 |
| creWNT1 | 2210408l21Rik | 0.000498802 |
| creWNT1 | Trpa1 | 0.000529901 |
| creWNT1 | 4930567K20Rik | 0.001270571 |
| creWNT1 | Cdk5rap2 | 0.001270571 |
| creWNT1 | 1700120E14Rik | 0.001745793 |
| creWNT1 | Megf6 | 0.001901194 |
| creWNT1 | 1700001G17Rik | 0.001978481 |
| creWNT1 | Mamdc2 | 0.002454858 |
| creWNT1 | Mir669h | 0.002572548 |
| creWNT1 | Ppp1r36 | 0.002596876 |
| creWNT1 | Gm10409 | 0.002612369 |
| creWNT1 | Tbxa2r | 0.003124112 |
| creWNT1 | Gm3500 | 0.00335704 |
| creWNT1 | Tbx21 | 0.00335704 |
| creWNT1 | E030003E18Rik | 0.003967941 |
| creWNT1 | Mir337 | 0.004850625 |
| creWNT1 | Naip7 | 0.007546055 |
| creWNT1 | F830016B08Rik | 0.009510925 |
| creWNT1 | Stard8 | 0.018514642 |
| creWNT1 | Il2ra | 0.018514642 |
| creWNT1 | Tinag | 0.019084356 |
| creWNT1 | 4930459L07Rik | 0.019084356 |
| creWNT1 | Gm2027 | 0.019084356 |
| creWNT1 | Litaf | 0.019084356 |
| creWNT1 | 4930555G01Rik | 0.027954627 |
| creWNT1 | Zdhhc11 | 0.027954627 |
| creWNT1 | BC048644 | 0.027954627 |
| creWNT1 | Fgf9 | 0.027972954 |
| creWNT1 | Mir669a-10 | 0.030214147 |
| creWNT1 | 4930432K09Rik | 0.035380602 |
| creWNT1 | Srsf2 | 0.036657657 |
| creWNT1 | Klhl14 | 0.038415208 |
| creWNT1 | Nrk | 0.039080173 |
| creWNT1 | Loxhd1 | 0.039456866 |
| creWNT1 | 1700046C09Rik | 0.043023116 |
| creWNT1 | D7Ertd443e | 0.043964887 |
| creWNT1 | Gnal | 0.043964887 |
| creWNT1 | Gypc | 0.044363418 |
| creWNT1 | Gm3383 | 0.046976444 |
| creWNT1 | Pole | 0.046976444 |
| genderF | Tsix | 0 |
| genderF | Xist | 0 |
| genderF | Uty | 0 |
| genderF | Gm20867 | 2.8816E−293 |
| genderF | Gm20738 | 2.1971E−292 |
| genderF | Gm20823 | 3.6653E−287 |
| genderF | Eif2s3y | 8.0506E−282 |
| genderF | Gm20816 | 1.1202E−281 |
| genderF | Gm20736 | 1.4483E−236 |
| genderF | Klk1b22 | 1.6261E−158 |
| genderF | Kdm5d | 1.0299E−146 |
| genderF | Gm13710 | 2.42887E−98 |
| genderF | Pisd-ps3 | 5.28451E−90 |
| genderF | Klk1b21 | 8.32343E−83 |
| genderF | Srsf2 | 8.84863E−75 |
| genderF | Klk1b24 | 1.24083E−70 |
| genderF | Gm20871 | 7.9467E−69 |
| genderF | Slc15a2 | 1.79311E−59 |
| genderF | Sft2d2 | 6.26851E−55 |
| genderF | Gm20854 | 1.05704E−36 |
| genderF | Klk1b27 | 4.81131E−33 |
| genderF | Sly | 5.89545E−33 |
| genderF | Tuba1c | 1.29992E−32 |
| genderF | Klk1b11 | 6.06754E−31 |
| genderF | Ulk4 | 1.92696E−29 |
| genderF | BC117090 | 4.44252E−26 |
| genderF | Rbm5 | 7.22375E−25 |
| genderF | Retnlb | 1.4118E−24 |
| genderF | Zfp69 | 1.86732E−24 |
| genderF | Hnrnpc | 2.7214E−24 |
| genderF | Adamts13 | 7.43422E−23 |
| genderF | Kdm6a | 2.62243E−22 |
| genderF | Ccrn4l | 3.60768E−21 |
| genderF | H2-Q5 | 5.39364E−21 |
| genderF | Gm3893 | 5.39364E−21 |
| genderF | H2-Q9 | 6.83468E−20 |
| genderF | 4932443I19Rik | 8.84372E−19 |
| genderF | 4933409K07Rik | 1.73157E−18 |
| genderF | Car1 | 7.27138E−18 |
| genderF | Fth1 | 1.33518E−17 |
| genderF | Gm8909 | 7.80276E−17 |
| genderF | Rn45s | 4.14997E−16 |
| genderF | Dlgap1 | 4.86945E−16 |
| genderF | H2-Q4 | 2.51145E−15 |
| genderF | Gpsm1 | 4.29472E−15 |
| genderF | Rftn1 | 1.13391E−14 |
| genderF | Mill2 | 1.15631E−13 |
| genderF | Celf3 | 2.11585E−13 |
| genderF | Selenbp1 | 4.13667E−13 |
| genderF | Fam163b | 6.01723E−13 |
| genderF | Gal | 7.02594E−13 |
| genderF | Guca2a | 7.19575E−13 |
| genderF | Picalm | 2.17293E−12 |
| genderF | H2-Q1 | 2.39041E−12 |
| genderF | Phgr1 | 2.45709E−12 |
| genderF | Bzrap1 | 2.45709E−12 |
| genderF | Ybx1 | 6.46464E−12 |
| genderF | Tmprss6 | 7.56943E−12 |
| genderF | Klk1b3 | 9.61136E−12 |
| genderF | Nlrp5-ps | 9.86982E−12 |
| genderF | Fras1 | 2.30727E−11 |
| genderF | Selenbp2 | 3.86985E−11 |
| genderF | Gpr19 | 4.73732E−11 |
| genderF | Loxl2 | 6.10962E−11 |
| genderF | Gm7120 | 1.0785E−10 |
| genderF | Zg16 | 1.08286E−10 |
| genderF | Rnf121 | 1.37718E−10 |
| genderF | Fabp2 | 1.91674E−10 |
| genderF | Klk1b1 | 6.03329E−10 |
| genderF | H2-Bl | 8.13666E−10 |
| genderF | Kcnh6 | 1.11854E−09 |
| genderF | Slc27a2 | 1.2876E−09 |
| genderF | DQ267100 | 1.47828E−09 |
| genderF | Prdx6b | 2.01581E−09 |
| genderF | B2m | 2.04809E−09 |
| genderF | Mgst1 | 7.7396E−09 |
| genderF | Hist2h2bb | 8.94628E−09 |
| genderF | E030019B13Rik | 9.33954E−09 |
| genderF | Magohb | 9.43109E−09 |
| genderF | Miip | 9.77397E−09 |
| genderF | Mptx1 | 1.37867E−08 |
| genderF | Poteg | 1.37867E−08 |
| genderF | Slc4a4 | 1.50896E−08 |
| genderF | Ccdc3 | 1.52029E−08 |
| genderF | 3110007F17Rik | 1.91748E−08 |
| genderF | Cnksr2 | 3.11545E−08 |
| genderF | Ptpro | 3.35981E−08 |
| genderF | Pirt | 4.65243E−08 |
| genderF | Prdx6 | 5.98586E−08 |
| genderF | Sod2 | 5.98586E−08 |
| genderF | Ndfip2 | 6.26697E−08 |
| genderF | Cldn3 | 6.94496E−08 |
| genderF | Pde6a | 8.1929E−08 |
| genderF | Csad | 8.70955E−08 |
| genderF | Kcnip1 | 8.95196E−08 |
| genderF | H2-M5 | 9.10283E−08 |
| genderF | 9330111N05Rik | 1.0249E−07 |
| genderF | Agr2 | 1.09098E−07 |
| genderF | Rps3a1 | 1.21709E−07 |
| genderF | Gm14525 | 1.23139E−07 |
| genderF | Ovch2 | 2.1328E−07 |
| genderF | Pla2g2a | 7.97488E−07 |
| genderF | Kcnk9 | 1.08163E−06 |
| genderF | Pla2g5 | 2.53393E−05 |
| genderF | Klk1b16 | 2.64395E−05 |
| genderF | Aldh1a1 | 3.21958E−05 |
| genderF | 4930447C04Rik | 3.51266E−05 |
| genderF | Slpi | 3.81138E−05 |
| genderF | Ccdc88c | 7.75044E−05 |
| genderF | Hist1h2ba | 9.77188E−05 |
| genderF | Ces1c | 0.000137196 |
| genderF | Lpo | 0.000153441 |
| genderF | Spink6 | 0.000216662 |
| genderF | Tspy-ps | 0.000266299 |
| genderF | Reg3g | 0.000281366 |
| genderF | Insrr | 0.000540425 |
| genderF | Ces2e | 0.000634637 |
| genderF | Adamdec1 | 0.000942276 |
| genderF | Olfr631 | 0.001712112 |
| genderF | Ces2a | 0.0018362 |
| genderF | Tlr8 | 0.001926716 |
| genderF | Slc38a3 | 0.00280639 |
| genderF | S100g | 0.003723584 |
| genderF | Scel | 0.003723584 |
| genderF | Gsdmc2 | 0.004161108 |
| genderF | Gm20750 | 0.004557207 |
| genderF | Srgn | 0.00484209 |
| genderF | Gm15760 | 0.005507258 |
| genderF | Hsd3b5 | 0.006034702 |
| genderF | 1810006J02Rik | 0.008906683 |
| genderF | Zdbf2 | 0.009214387 |
| genderF | Mal | 0.009362334 |
| genderF | Gm10057 | 0.010474958 |
| genderF | Gm14492 | 0.010913036 |
| genderF | Sh3bp2 | 0.012359271 |
| genderF | Wnt2 | 0.012780624 |
| genderF | Tex40 | 0.013290824 |
| genderF | Speer1-ps1 | 0.014137867 |
| genderF | Pyroxd2 | 0.015638802 |
| genderF | Prss30 | 0.016005394 |
| genderF | Hsd17b2 | 0.017135393 |
| genderF | Tmprss5 | 0.017864893 |
| genderF | Gm4925 | 0.018934066 |
| genderF | Ces2c | 0.019055403 |
| genderF | 0610007N19Rik | 0.019137519 |
| genderF | Defb39 | 0.021498302 |
| genderF | E030044B06Rik | 0.022176689 |
| genderF | D5Ertd577e | 0.024572843 |
| genderF | Clec3b | 0.025090447 |
| genderF | Phldb2 | 0.027315419 |
| genderF | Slc25a41 | 0.031031026 |
| genderF | Klk1b5 | 0.033619031 |
| genderF | Cyp2r1 | 0.034330497 |
| genderF | Gabrr1 | 0.034737168 |
| genderF | BC064078 | 0.037994863 |
| genderF | Astl | 0.03831308 |
| genderF | Tmem170 | 0.039154133 |
| genderF | Tbata | 0.039242099 |
| genderF | Ikbke | 0.039242099 |
| genderF | Dpep1 | 0.039251396 |
| genderF | Pcdhb7 | 0.039437361 |
| genderF | Mir5109 | 0.040626516 |
| genderF | Cd70 | 0.044843484 |
| genderF | Lgi4 | 0.044892382 |
| genderF | Casq1 | 0.048056803 |
| time7PM | Per3 | 5.19873E−62 |
| time7PM | Arntl | 7.18728E−54 |
| time7PM | Nr1d2 | 8.43445E−30 |
| time7PM | Tef | 3.2917E−20 |
| time7PM | Per2 | 3.48865E−16 |
| time7PM | Rgs4 | 1.87781E−12 |
| time7PM | Ppia | 4.01694E−12 |
| time7PM | Scg2 | 4.75066E−12 |
| time7PM | Ckb | 5.66075E−12 |
| time7PM | Pcsk1n | 6.30192E−12 |
| time7PM | Rpl3 | 1.05766E−11 |
| time7PM | Slc25a4 | 1.30729E−11 |
| time7PM | Rps20 | 1.44938E−11 |
| time7PM | Rps3a1 | 1.87538E−11 |
| time7PM | Tpt1 | 1.87538E−11 |
| time7PM | Olfm1 | 1.89635E−11 |
| time7PM | Prph | 4.57481E−11 |
| time7PM | 1500032L24Rik | 5.12427E−11 |
| time7PM | Cst3 | 5.55487E−11 |
| time7PM | Gm13498 | 5.55487E−11 |
| time7PM | Srsf2 | 5.66263E−11 |
| time7PM | Tubb2a | 5.74992E−11 |
| time7PM | Cfl1 | 5.74992E−11 |
| time7PM | Map1lc3a | 1.01946E−10 |
| time7PM | Cd80 | 2.10894E−10 |
| time7PM | Chchd2 | 2.95065E−10 |
| time7PM | Nap1l5 | 3.01022E−10 |
| time7PM | Bex2 | 4.7105E−10 |
| time7PM | Rps23 | 5.65576E−10 |
| time7PM | Rplp2-ps1 | 6.27462E−10 |
| time7PM | Cd81 | 1.10114E−09 |
| time7PM | Rasl2-9 | 1.17794E−09 |
| time7PM | Atp6v0c | 1.24843E−09 |
| time7PM | Oaz1 | 2.21838E−09 |
| time7PM | BC147527 | 3.94126E−09 |
| time7PM | Reep5 | 4.68766E−09 |
| time7PM | Slc7a11 | 5.37567E−09 |
| time7PM | Rpl7 | 5.37567E−09 |
| time7PM | Slc1a1 | 5.91408E−09 |
| time7PM | Plat | 7.30632E−09 |
| time7PM | Hnrnpk | 7.30632E−09 |
| time7PM | Skint10 | 7.30632E−09 |
| time7PM | Ndrg4 | 7.91227E−09 |
| time7PM | Itga9 | 8.05619E−09 |
| time7PM | Eef1a1 | 1.03687E−08 |
| time7PM | Ngfrap1 | 1.09681E−08 |
| time7PM | Actg1 | 1.09681E−08 |
| time7PM | Eef2 | 1.09681E−08 |
| time7PM | 1700034F02Rik | 1.09681E−08 |
| time7PM | Srrm2 | 1.32471E−08 |
| time7PM | Zfp712 | 1.36428E−08 |
| time7PM | Chrna3 | 1.58211E−08 |
| time7PM | Prdx2 | 1.80085E−08 |
| time7PM | Zfp708 | 2.17839E−08 |
| time7PM | 1700016L04Rik | 2.17839E−08 |
| time7PM | Tuba1b | 2.25733E−08 |
| time7PM | Aldoart1 | 2.34208E−08 |
| time7PM | Vat1 | 2.41603E−08 |
| time7PM | Ndn | 2.44194E−08 |
| time7PM | Skint6 | 2.82101E−08 |
| time7PM | Magee1 | 2.93161E−08 |
| time7PM | Aldoart2 | 2.99209E−08 |
| time7PM | Rnasek | 3.08044E−08 |
| time7PM | Cxx1c | 3.2434E−08 |
| time7PM | Tubb3 | 3.73276E−08 |
| time7PM | Gm5148 | 3.78794E−08 |
| time7PM | Rimklb | 3.88177E−08 |
| time7PM | Rpl31-ps12 | 3.94705E−08 |
| time7PM | Rfx2 | 4.41387E−08 |
| time7PM | Dbp | 4.74225E−08 |
| time7PM | Ywhaq | 5.03044E−08 |
| time7PM | Ndufa2 | 6.65414E−08 |
| time7PM | Cox8a | 6.65414E−08 |
| time7PM | Gm9079 | 7.43157E−08 |
| time7PM | Cox4i1 | 8.92263E−08 |
| time7PM | Tnfsf4 | 9.74321E−08 |
| time7PM | Vps13a | 1.00194E−07 |
| time7PM | Tspan3 | 1.00211E−07 |
| time7PM | 2210404O07Rik | 1.04649E−07 |
| time7PM | Pla2g4c | 1.04649E−07 |
| time7PM | Gm129 | 1.05284E−07 |
| time7PM | Banp | 1.11336E−07 |
| time7PM | Chrnb4 | 1.15915E−07 |
| time7PM | Slc25a39 | 1.17567E−07 |
| time7PM | Rpl14 | 1.18238E−07 |
| time7PM | Fau | 1.18238E−07 |
| time7PM | Emc10 | 1.30289E−07 |
| time7PM | Pgam1 | 1.31912E−07 |
| time7PM | Ubb | 1.53672E−07 |
| time7PM | Hist1h2bf | 1.58901E−07 |
| time7PM | Syt4 | 1.58901E−07 |
| time7PM | Gm12070 | 1.58901E−07 |
| time7PM | Flot1 | 1.60309E−07 |
| time7PM | Gm11978 | 1.86443E−07 |
| time7PM | Gm6548 | 2.07259E−07 |
| time7PM | Per1 | 2.20958E−07 |
| time7PM | Gm6682 | 2.20958E−07 |
| time7PM | H3f3b | 2.20958E−07 |
| time7PM | Nedd8 | 2.35247E−07 |
| time7PM | Vamp2 | 2.60795E−07 |
| time7PM | Lgals1 | 3.23817E−07 |
| time7PM | B3gn1l | 4.25683E−07 |
| time7PM | Hist1h2bb | 8.41838E−07 |
| time7PM | 4931408D14Rik | 1.87191E−06 |
| time7PM | Hist1h2bm | 3.25781E−06 |
| time7PM | Gm4980 | 3.49497E−06 |
| time7PM | Tubb2a-ps2 | 3.91079E−06 |
| time7PM | Zbtbl6 | 4.21897E−06 |
| time7PM | 2310047M10Rik | 3.05115E−05 |
| time7PM | Tnfsf18 | 4.48974E−05 |
| time7PM | Olfr631 | 7.7947E−05 |
| time7PM | Gm7977 | 0.000345879 |
| time7PM | Wfs1 | 0.000633638 |
| time7PM | Gm15941 | 0.000806771 |
| time7PM | Uts2 | 0.001578899 |
| time7PM | Serpine2 | 0.001648971 |
| time7PM | Sstr2 | 0.00177418 |
| time7PM | Nrsn2 | 0.001803124 |
| time7PM | Cdh19 | 0.00188134 |
| time7PM | 2010109I03Rik | 0.002657443 |
| time7PM | Mep1a | 0.002683042 |
| time7PM | Otoa | 0.002739061 |
| time7PM | Gm14525 | 0.002911147 |
| time7PM | Rec8 | 0.002977444 |
| time7PM | Cck | 0.002981295 |
| time7PM | Mrgpre | 0.003366782 |
| time7PM | Tmem35 | 0.003592314 |
| time7PM | 1700003E16Rik | 0.003602257 |
| time7PM | Hspb1 | 0.003602257 |
| time7PM | Rps4y2 | 0.003900878 |
| time7PM | Insm2 | 0.003900878 |
| time7PM | Mt3 | 0.005469945 |
| time7PM | Fjx1 | 0.006599597 |
| time7PM | Hrh3 | 0.006712859 |
| time7PM | Adora1 | 0.006922857 |
| time7PM | BC049762 | 0.008130523 |
| time7PM | Slc26a3 | 0.008474715 |
| time7PM | Klk1b3 | 0.008948655 |
| time7PM | Vmn2r52 | 0.00993029 |
| time7PM | Gbp11 | 0.010130941 |
| time7PM | Kbtbd7 | 0.010702991 |
| time7PM | Arsj | 0.010809842 |
| time7PM | Klk1b21 | 0.011493587 |
| time7PM | Ngfr | 0.011755879 |
| time7PM | Ces1b | 0.011982297 |
| time7PM | Hsd3b5 | 0.012488186 |
| time7PM | Gm20753 | 0.014226719 |
| time7PM | Gm6588 | 0.014570697 |
| time7PM | Ddc | 0.015274067 |
| time7PM | Ccdc88c | 0.015592684 |
| time7PM | Rad51ap2 | 0.015717409 |
| time7PM | Klf15 | 0.015863914 |
| time7PM | Gtsf1 | 0.015923358 |
| time7PM | Ncrna00086 | 0.015954763 |
| time7PM | Il13ra1 | 0.01646116 |
| time7PM | Ikzf1 | 0.016590373 |
| time7PM | Pttg1 | 0.016683837 |
| time7PM | 4930564C03Rik | 0.019189435 |
| time7PM | Sstr3 | 0.019420943 |
| time7PM | Pgk2 | 0.019470565 |
| time7PM | Klk1b22 | 0.020743647 |
| time7PM | Al504432 | 0.021135189 |
| time7PM | Reg3g | 0.021654862 |
| time7PM | Phxr4 | 0.022335477 |
| time7PM | Dclk3 | 0.022525958 |
| time7PM | Rasal3 | 0.022530636 |
| time7PM | Cd177 | 0.023585934 |
| time7PM | 4930503O07Rik | 0.023819512 |
| time7PM | Adamts19 | 0.026662255 |
| time7PM | AU019990 | 0.027841476 |
| time7PM | Gm14015 | 0.029305401 |
| time7PM | Grp | 0.03018402 |
| time7PM | Stk32b | 0.030342447 |
| time7PM | Hus1b | 0.03182747 |
| time7PM | Mad2l1 | 0.032326524 |
| time7PM | Tex28 | 0.03299506 |
| time7PM | Aldh1a1 | 0.033261856 |
| time7PM | Calcb | 0.033261856 |
| time7PM | Birc5 | 0.034043722 |
| time7PM | Kcna5 | 0.034096188 |
| time7PM | Dll1 | 0.034237457 |
| time7PM | 4930598F16Rik | 0.034667601 |
| time7PM | 1700009C05Rik | 0.035480569 |
| time7PM | Tal1 | 0.036554406 |
| time7PM | Gabre | 0.038750127 |
| time7PM | Klk1b24 | 0.040016715 |
| time7PM | Cidea | 0.040482335 |
| time7PM | Cml3 | 0.043405043 |
| time7PM | Mr1 | 0.044196594 |
| time7PM | Lrrc18 | 0.046015378 |
| locationDistal | 1810065E05Rik | 5.0302E−71 |
| locationDistal | Col5a3 | 1.79412E−20 |
| locationDistal | Guca2a | 9.79186E−26 |
| locationDistal | Muc2 | 5.0291E−123 |
| locationDistal | Dmbt1 | 1.02141E−18 |
| locationDistal | Hmgcs2 | 3.45357E−22 |
| locationDistal | Thy1 | 1.31805E−11 |
| locationDistal | Ltk | 0.000140038 |
| locationDistal | Col27a1 | 2.42041E−09 |
| locationDistal | 5930412G12Rik | 0.000215047 |
| locationDistal | Rapgef4 | 3.47654E−15 |
| locationDistal | Ceacam1 | 9.56246E−16 |
| locationDistal | 4930443O20Rik | 1.23225E−11 |
| locationDistal | Itpr1 | 3.84226E−09 |
| locationDistal | Stxbp6 | 9.7137E−07 |
| locationDistal | Snca | 4.1084E−10 |
| locationDistal | Gcnt3 | 1.46163E−12 |
| locationDistal | Spock1 | 2.34254E−14 |
| locationDistal | 2310067B10Rik | 7.15354E−08 |
| locationDistal | Gm5607 | 0.000261527 |
| locationDistal | Cyp2c55 | 1.19148E−13 |
| locationDistal | Itga8 | 6.54859E−10 |
| locationDistal | Car1 | 4.65878E−23 |
| locationDistal | Vat1l | 3.45552E−14 |
| locationDistal | Lin7a | 4.8049E−09 |
| locationDistal | Reg3g | 2.6867E−08 |
| locationDistal | Trim9 | 8.2977E−06 |
| locationDistal | Syt6 | 0.000142955 |
| locationDistal | Cdr1 | 3.29476E−07 |
| locationDistal | Pex5l | 3.19064E−05 |
| locationDistal | Cacna1h | 1.54266E−05 |
| locationDistal | Tnfsf4 | 0.002705121 |
| locationDistal | Retnlb | 2.63419E−12 |
| locationDistal | Ptpro | 4.21106E−06 |
| locationDistal | Ephb1 | 1.07692E−09 |
| locationDistal | Kcnh7 | 5.84873E−07 |
| locationDistal | Drp2 | 3.44848E−05 |
| locationDistal | Unc5d | 6.68818E−08 |
| locationDistal | 2810032G03Rik | 2.30157E−06 |
| locationDistal | Gmip | 0.00012201 |
| locationDistal | Adcy1 | 0.001280619 |
| locationDistal | Tle1 | 0.000147795 |
| locationDistal | Spred3 | 5.38764E−06 |
| locationDistal | Gpr176 | 1.46439E−05 |
| locationDistal | Usp35 | 0.000439112 |
| locationDistal | Kcnk3 | 2.79654E−05 |
| locationDistal | Dzip1l | 0.000251404 |
| locationDistal | Kcnj6 | 1.39606E−06 |
| locationDistal | Fabp2 | 4.95157E−15 |
| locationDistal | Gm5424 | 0.001021026 |
| locationDistal | Lmx1b | 2.21744E−06 |
| locationDistal | 4833424O15Rik | 1.69837E−06 |
| locationDistal | Gm21949 | 7.00341E−05 |
| locationDistal | Pparg | 3.30454E−07 |
| locationDistal | Slc9a9 | 0.000009044 |
| locationDistal | Ncald | 2.43406E−11 |
| locationDistal | Dlc1 | 2.53692E−07 |
| locationDistal | Pdia5 | 9.16607E−05 |
| locationDistal | 4931430N09Rik | 1.48831E−05 |
| locationDistal | Pde1c | 9.50749E−10 |
| locationDistal | Nell1 | 8.59222E−08 |
| locationDistal | Wipf1 | 0.0008214 |
| locationDistal | Ipw | 6.46278E−07 |
| locationDistal | Clvs2 | 7.87803E−10 |
| locationDistal | Aebp1 | 0.012043316 |
| locationDistal | Epn1 | 1.51932E−05 |
| locationDistal | Lrrc16b | 0.000279829 |
| locationDistal | Cacna1c | 2.78953E−13 |
| locationDistal | Atxn2 | 6.23173E−11 |
| locationDistal | Lmtk3 | 5.20768E−07 |
| locationDistal | Car10 | 9.45705E−08 |
| locationDistal | Speg | 2.50992E−07 |
| locationDistal | Cttnbp2 | 0.000172105 |
| locationDistal | Vwa5b1 | 0.000031955 |
| locationDistal | Map7d1 | 0.001682708 |
| locationDistal | Sh3rf1 | 5.45096E−06 |
| locationDistal | Mki67 | 0.000185017 |
| locationDistal | Emp1 | 0.003479236 |
| locationDistal | Hs6st1 | 0.010483316 |
| locationDistal | Syne2 | 0.001677064 |
| locationDistal | Bri3 | 0.001679786 |
| locationDistal | Arhgap42 | 3.12067E−06 |
| locationDistal | Epha7 | 2.08181E−05 |
| locationDistal | Mtmr1 | 0.000911969 |
| locationDistal | Dcc | 0.000048386 |
| locationDistal | Cnga3 | 0.008187081 |
| locationDistal | Rtkn | 0.000689193 |
| locationDistal | Card10 | 0.031186874 |
| locationDistal | Tgfb2 | 0.001181946 |
| locationDistal | Slc30a10 | 0.041784807 |
| locationDistal | Spns2 | 0.000572287 |
| locationDistal | St3gal1 | 1.23051E−08 |
| locationDistal | Il31ra | 6.175E−12 |
| locationDistal | Arhgap10 | 0.000361424 |
| locationDistal | Fat3 | 5.46203E−05 |
| locationDistal | Ttyh3 | 5.05248E−07 |
| locationDistal | Hgf | 0.020503301 |
| locationDistal | Trim25 | 0.002030274 |
| locationDistal | Tmem245 | 0.00294364 |
| locationDistal | Cnnm1 | 2.86069E−05 |
| locationDistal | Slc18a3 | 1.04564E−17 |
| locationDistal | Hoxb13 | 7.45641E−17 |
| locationDistal | Ffar3 | 3.7311E−17 |
| locationDistal | Sycn | 1.06115E−24 |
| locationDistal | Adh1 | 1.6346E−32 |
| locationDistal | Saa1 | 4.34575E−21 |
| locationDistal | Car4 | 8.29501E−18 |
| locationDistal | Pmepa1 | 2.56399E−18 |
| locationDistal | Vstm4 | 2.78601E−15 |
| locationDistal | Stmn1 | 1.65275E−18 |
| locationDistal | Susd5 | 2.21536E−25 |
| locationDistal | Nefl | 5.55625E−16 |
| locationDistal | Chgb | 1.25167E−18 |
| locationDistal | Ncam1 | 3.06112E−15 |
| locationDistal | Skint10 | 9.10951E−22 |
| locationDistal | Ddx5 | 2.3161E−24 |
| locationDistal | Dpysl2 | 3.7311E−17 |
| locationDistal | Gm12504 | 9.42966E−16 |
| locationDistal | Hspa5 | 1.128E−15 |
| locationDistal | Prkar1a | 1.87598E−17 |
| locationDistal | 9330111N05Rik | 3.42945E−17 |
| locationDistal | Hnrnpa2b1 | 1.71251E−17 |
| locationDistal | Calr | 1.03054E−15 |
| locationDistal | Map1b | 1.00008E−23 |
| locationDistal | Gm13498 | 1.12451E−22 |
| locationDistal | Atp6v0c | 1.14896E−24 |
| locationDistal | Cfl1 | 2.86766E−15 |
| locationDistal | Srsf5 | 4.00949E−17 |
| locationDistal | Calm1 | 2.75276E−15 |
| locationDistal | Actb | 1.87352E−15 |
| locationDistal | Pgam1 | 2.64348E−21 |
| locationDistal | Calm2 | 1.15628E−16 |
| locationDistal | Chrna3 | 1.73066E−15 |
| locationDistal | Tagln2 | 1.12992E−15 |
| locationDistal | Cd80 | 3.45357E−22 |
| locationDistal | Prdx6b | 6.09396E−77 |
| locationDistal | Htr3b | 6.86195E−19 |
| locationDistal | Gm19782 | 1.68069E−25 |
| locationDistal | Zfp708 | 6.32465E−22 |
| locationDistal | Ckb | 4.77236E−17 |
| locationDistal | Gapdh | 2.78564E−15 |
| locationDistal | Hsp90ab1 | 4.49299E−16 |
| locationDistal | Lypd8 | 9.12116E−27 |
| locationDistal | Ngfr | 1.90527E−16 |
| locationDistal | Tppp3 | 2.97386E−15 |
| locationDistal | Oaz1 | 4.0001E−13 |
| locationDistal | Plekha7 | 7.59332E−08 |
| locationDistal | Slc25a4 | 6.20713E−14 |
| locationDistal | Ephx1 | 6.38082E−10 |
| locationDistal | Dstn | 2.53679E−11 |
| locationDistal | Aldoart2 | 2.80512E−31 |
| locationDistal | Fth1 | 1.50153E−07 |
| locationDistal | Tmem176b | 9.05029E−12 |
| locationDistal | Moxd1 | 4.47882E−12 |
| locationDistal | Ubb | 1.84237E−21 |
| locationDistal | Tmx2 | 1.39932E−14 |
| locationDistal | Gas6 | 1.42101E−13 |
| locationDistal | Psmd13 | 2.19629E−10 |
| locationDistal | Tubb2a | 3.08863E−13 |
| locationDistal | Cd81 | 3.06112E−15 |
| locationDistal | Ppia | 4.71693E−14 |
| locationDistal | Tubb5 | 2.16838E−16 |
| locationDistal | Atp5b | 4.0001E−13 |
| locationDistal | Serinc1 | 4.71283E−11 |
| locationDistal | Faim2 | 1.128E−15 |
| locationDistal | Pdzd2 | 1.60902E−16 |
| locationDistal | Skil | 3.49607E−22 |
| locationDistal | Tuba1a | 2.33422E−27 |
| locationDistal | Ngb | 1.6377E−06 |
| locationDistal | Ubc | 1.43121E−17 |
| locationDistal | Crip1 | 2.89977E−10 |
| locationDistal | Pcsk1n | 1.28637E−14 |
| locationDistal | Fgf14 | 4.29965E−33 |
| locationDistal | Cst3 | 5.23179E−17 |
| locationDistal | H3f3b | 2.50429E−17 |
| locationDistal | Slc7a11 | 2.69319E−40 |
| locationDistal | Sprr2a2 | 2.80341E−12 |
| locationDistal | Hsp90b1 | 1.06719E−16 |
| locationDistal | Dusp3 | 4.9835E−13 |
| locationDistal | 2610017I09Rik | 6.38082E−10 |
| locationDistal | 1700016L04Rik | 3.63201E−32 |
| locationDistal | Aldoa | 2.24053E−24 |
| locationDistal | Aldoart1 | 2.65567E−32 |
| locationDistal | Mid1 | 1.61409E−15 |
| locationDistal | Vip | 6.8116E−16 |
| locationDistal | S100a6 | 8.84934E−21 |
| locationDistal | Sprr2a1 | 1.49715E−12 |
| locationDistal | Frmd5 | 5.84034E−20 |
| locationDistal | Gm6548 | 6.73722E−27 |
| locationDistal | Tmem255b | 8.31669E−09 |
| locationDistal | Eef1a1 | 5.71406E−24 |
| locationDistal | Canx | 8.24407E−12 |
| locationDistal | Cd9 | 1.57612E−19 |
| locationDistal | Slc35d3 | 0.002088855 |
| locationDistal | Scn5a | 4.17905E−10 |
| locationDistal | 6330403K07Rik | 1.2319E−30 |
| locationDistal | Gm12070 | 2.38431E−24 |
| locationDistal | Kcnq5 | 6.25436E−35 |
| locationDistal | Nell2 | 1.99749E−33 |
| locationDistal | Slc1a1 | 2.8046E−22 |
| locationDistal | Syt2 | 4.89713E−23 |
| locationDistal | Vmn2r-ps54 | 4.69693E−48 |
| locationDistal | Ctsb | 1.49754E−23 |
| locationDistal | Itm2b | 2.15884E−19 |
| locationDistal | Epha8 | 4.99284E−07 |
| locationDistal | Actg1 | 8.16605E−11 |
| locationDistal | Chrm1 | 1.49258E−10 |
| locationDistal | Slc10a4 | 9.65963E−18 |
| locationDistal | Scg2 | 1.654E−24 |
| locationDistal | Gm6682 | 1.67156E−36 |
| locationDistal | Gm1821 | 3.73976E−24 |
| locationDistal | Ldlrad4 | 4.08505E−42 |
| locationDistal | Tuba1b | 4.15124E−25 |
| locationDistal | Hspa8 | 3.27521E−31 |
| locationDistal | Dkk3 | 3.06472E−54 |
| locationDistal | Prdx6 | 9.51863E−60 |
| locationDistal | Prnp | 1.41056E−27 |
| locationDistal | Rgs9 | 1.03541E−29 |
| locationDistal | Bglap | 5.83044E−19 |
| locationDistal | Htr3a | 5.25799E−52 |
| locationDistal | Il22ra2 | 0.00270681 |
| locationDistal | Hoxd13 | 0.003043785 |
| locationDistal | Nobox | 0.009799842 |
| locationDistal | Fam115e | 0.011774848 |
| locationDistal | Tcf21 | 0.013858468 |
| locationDistal | Btbd17 | 0.016327774 |
| locationDistal | S100a5 | 0.023025186 |
| locationDistal | Krt1 | 0.023421018 |
| locationDistal | Hbb-b1 | 0.028663261 |
| locationDistal | Agtr2 | 0.031135797 |
| locationDistal | Olfr55 | 0.031811195 |
| locationDistal | Slc34a1 | 0.035725695 |
| locationDistal | Tas2r108 | 0.03627001 |
| locationDistal | Eppin | 0.036602586 |
| locationDistal | Olfr1352 | 0.038091947 |
| locationDistal | Otor | 0.038669996 |
| locationDistal | Sftpc | 0.039119673 |
| locationDistal | Mup16 | 0.043704727 |
| locationDistal | H1fnt | 0.044551941 |
| locationDistal | Prnd | 0.044868375 |
| locationDistal | H19 | 0.046982862 |
| locationDistal | Nts | 0.047727188 |

TABLE 16

| ident | gene | padjH |
|---|---|---|
| Glia_1 | Etl4 | 2.6493E−207 |
| Glia_1 | Agbl4 | 3.8197E−193 |
| Glia_1 | Hmcn1 | 1.3015E−167 |
| Glia_1 | Kank1 | 1.2357E−132 |
| Glia_1 | Lsamp | 1.5045E−118 |
| Glia_1 | AW549542 | 4.7263E−112 |
| Glia_1 | Auts2 | 3.7028E−110 |
| Glia_1 | Cpe | 5.8938E−105 |
| Glia_1 | Col9a2 | 6.80903E−98 |
| Glia_1 | Fam184b | 1.16823E−90 |
| Glia_1 | Bcan | 1.49796E−89 |
| Glia_1 | Cdc14a | 4.35941E−87 |
| Glia_1 | Pxdn | 4.47141E−86 |
| Glia_1 | Erc2 | 4.70356E−86 |
| Glia_1 | Mapk10 | 2.04324E−82 |
| Glia_1 | 2810055G20Rik | 2.04324E−82 |
| Glia_1 | Adarb2 | 4.7573E−81 |
| Glia_1 | Kif21a | 6.2727E−81 |
| Glia_1 | Zfpm2 | 8.21426E−81 |
| Glia_1 | Foxp2 | 1.07084E−76 |
| Glia_1 | Bai3 | 2.19602E−75 |
| Glia_1 | Slc18a2 | 5.56287E−71 |
| Glia_1 | Grid1 | 3.23449E−69 |
| Glia_1 | Sfxn5 | 1.89025E−67 |
| Glia_1 | Pde3a | 1.11362E−66 |
| Glia_1 | Trim9 | 9.4315E−66 |
| Glia_1 | Plxna4 | 4.29509E−60 |
| Glia_1 | Hmgcll1 | 4.08361E−59 |
| Glia_1 | Nrg3 | 9.07368E−57 |
| Glia_1 | Tspan18 | 6.07183E−54 |
| Glia_1 | Dkk3 | 6.19463E−54 |
| Glia_1 | Rgs9 | 1.28429E−52 |
| Glia_1 | Ncam2 | 1.68797E−52 |
| Glia_1 | Nckap5 | 7.11858E−52 |
| Glia_1 | Kctd1 | 4.4845E−51 |
| Glia_1 | Zfp423 | 2.36683E−49 |
| Glia_1 | Gpc6 | 2.48969E−49 |
| Glia_1 | Hecw2 | 4.83158E−49 |
| Glia_1 | Sorl1 | 2.11189E−47 |
| Glia_1 | Ccdc148 | 2.30674E−47 |
| Glia_1 | Tshz2 | 7.95663E−47 |
| Glia_1 | Airn | 7.99303E−46 |
| Glia_1 | Fam5c | 2.06954E−45 |
| Glia_1 | Enkur | 6.1597E−45 |
| Glia_1 | Gpam | 4.98277E−44 |
| Glia_1 | Col8a1 | 1.8472E−43 |
| Glia_1 | Rhbdl3 | 2.46025E−42 |
| Glia_1 | Cacng4 | 5.63167E−41 |
| Glia_1 | Ccdc164 | 9.78126E−41 |
| Glia_1 | Ext1 | 4.71115E−40 |
| Glia_1 | Rap1gap | 9.38064E−40 |
| Glia_1 | Tacr3 | 2.18492E−39 |
| Glia_1 | Bzrap1 | 5.04049E−39 |
| Glia_1 | Pde4d | 5.04049E−39 |
| Glia_1 | Armc2 | 9.68774E−39 |
| Glia_1 | Igfbp4 | 1.62816E−38 |
| Glia_1 | Cml3 | 2.99284E−38 |
| Glia_1 | Msi2 | 1.89266E−37 |
| Glia_1 | Sned1 | 8.29944E−37 |
| Glia_1 | Sulf1 | 9.0677E−37 |
| Glia_1 | Tprkb | 1.33089E−36 |
| Glia_1 | Apoe | 2.83612E−36 |
| Glia_1 | C230004F18Rik | 6.50039E−36 |
| Glia_1 | Cadps | 2.15901E−35 |
| Glia_1 | Lrriq1 | 4.50061E−35 |
| Glia_1 | Bai2 | 1.45354E−34 |
| Glia_1 | Arhgap42 | 1.78474E−34 |
| Glia_1 | Ulk4 | 7.88379E−34 |
| Glia_1 | Ctnna3 | 1.83837E−33 |
| Glia_1 | Ncdn | 3.24943E−33 |
| Glia_1 | Pitpnc1 | 4.37316E−33 |
| Glia_1 | Lrrc9 | 8.6402E−33 |
| Glia_1 | Igf2r | 1.04023E−32 |
| Glia_1 | Smoc1 | 1.52193E−32 |
| Glia_1 | Itga8 | 1.13278E−31 |
| Glia_1 | Plcb1 | 1.4539E−31 |
| Glia_1 | Cpxm2 | 4.64102E−31 |
| Glia_1 | Alcam | 5.56399E−31 |
| Glia_1 | Ntm | 7.4704E−31 |
| Glia_1 | Zfhx4 | 8.51794E−31 |
| Glia_1 | Tes | 1.1166E−30 |
| Glia_1 | Frzb | 1.16382E−30 |
| Glia_1 | Cntfr | 2.91649E−30 |
| Glia_1 | A330076C08Rik | 3.23789E−30 |
| Glia_1 | Ramp1 | 4.2952E−30 |
| Glia_1 | Creg2 | 5.77247E−30 |
| Glia_1 | Greb1 | 2.25352E−29 |
| Glia_1 | Fmo1 | 6.81302E−29 |
| Glia_1 | Hey2 | 1.41074E−28 |
| Glia_1 | Col11a1 | 2.06893E−28 |
| Glia_1 | Mterfd2 | 1.1382E−27 |
| Glia_1 | Dlg2 | 1.78281E−27 |
| Glia_1 | Mcc | 1.87011E−27 |
| Glia_1 | Fstl4 | 3.04287E−27 |
| Glia_1 | Fbln5 | 4.57858E−27 |
| Glia_1 | Ptgfrn | 5.00395E−27 |
| Glia_1 | Gria4 | 5.50254E−27 |
| Glia_1 | Mapk15 | 5.71885E−27 |
| Glia_1 | Sox2ot | 9.54627E−27 |
| Glia_1 | Ptprg | 1.7758E−26 |
| Glia_1 | Sgsm1 | 4.54204E−23 |
| Glia_1 | Tekt1 | 1.01448E−22 |
| Glia_1 | Ttc21a | 5.27234E−22 |
| Glia_1 | Lrp8 | 3.35532E−21 |
| Glia_1 | Kndc1 | 1.37146E−20 |
| Glia_1 | Gm216 | 3.9437E−20 |
| Glia_1 | Dnahc11 | 8.82409E−20 |
| Glia_1 | Cthrc1 | 6.03368E−18 |
| Glia_1 | Ccdc108 | 1.62567E−16 |
| Glia_1 | Ntsr1 | 5.97769E−15 |
| Glia_1 | Omg | 6.76777E−13 |
| Glia_1 | Dnaaf3 | 1.27183E−12 |
| Glia_1 | Ccdc40 | 1.02017E−11 |
| Glia_1 | Otor | 2.46665E−11 |
| Glia_1 | Wdr69 | 5.37382E−11 |
| Glia_1 | Slc1a3 | 1.36148E−10 |
| Glia_1 | Slc7a10 | 9.61106E−10 |
| Glia_1 | Rgs7bp | 9.92033E−10 |
| Glia_1 | 1500002O10Rik | 1.29923E−09 |
| Glia_1 | Fzd6 | 1.30968E−09 |
| Glia_1 | 1700003M07Rik | 3.54314E−09 |
| Glia_1 | Ccdc135 | 2.02305E−08 |
| Glia_1 | Tecta | 3.19405E−08 |
| Glia_1 | Tmem255b | 3.69528E−08 |
| Glia_1 | 6430531B16Rik | 6.39635E−08 |
| Glia_1 | Ddo | 1.20134E−07 |
| Glia_1 | P2rx6 | 1.44631E−07 |
| Glia_1 | Shisa7 | 3.20791E−07 |
| Glia_1 | Elmod1 | 2.38839E−06 |
| Glia_1 | Wdr16 | 5.07725E−06 |
| Glia_1 | 1700001C19Rik | 7.13366E−06 |
| Glia_1 | Efcab1 | 7.44406E−06 |
| Glia_1 | 1110017D15Rik | 1.09666E−05 |
| Glia_1 | Caps2 | 1.17523E−05 |
| Glia_1 | 4933436C20Rik | 1.28628E−05 |
| Glia_1 | Mlf1 | 1.7278E−05 |
| Glia_1 | Meig1 | 2.42364E−05 |
| Glia_1 | Ppp2r2c | 3.11918E−05 |
| Glia_1 | Vipr2 | 4.13931E−05 |
| Glia_1 | Ptx4 | 7.26245E−05 |
| Glia_1 | Slc7a8 | 0.000126748 |
| Glia_1 | D130043K22Rik | 0.000140838 |
| Glia_1 | Tex26 | 0.000158839 |
| Glia_1 | Aif1l | 0.000210577 |
| Glia_1 | Ankrd66 | 0.000228033 |
| Glia_1 | Slc7a4 | 0.000432775 |
| Glia_1 | Ppp1r1a | 0.000482375 |
| Glia_1 | C530044C16Rik | 0.000646068 |
| Glia_1 | E030019B06Rik | 0.000748804 |
| Glia_1 | Ankrd45 | 0.001041986 |
| Glia_1 | Rsph4a | 0.001322708 |
| Glia_1 | Ubxn10 | 0.001537994 |
| Glia_1 | Iqca | 0.00161044 |
| Glia_1 | Tnni3 | 0.001912103 |
| Glia_1 | Lect1 | 0.00317526 |
| Glia_1 | Oprk1 | 0.003362741 |
| Glia_1 | 1700007K13Rik | 0.004157784 |
| Glia_1 | B4galnt4 | 0.004365071 |
| Glia_1 | Arhgap8 | 0.004767805 |
| Glia_1 | AU022754 | 0.004863659 |
| Glia_1 | Bex4 | 0.008379063 |
| Glia_1 | Igsf1 | 0.010469209 |
| Glia_1 | Ssxb5 | 0.010867103 |
| Glia_1 | Ssxb3 | 0.012876761 |
| Glia_1 | Olfr267 | 0.013190464 |
| Glia_1 | Cited1 | 0.013306834 |
| Glia_1 | Ftsj2 | 0.014441779 |
| Glia_1 | Dmkn | 0.015250648 |
| Glia_1 | F2rl1 | 0.01918841 |
| Glia_1 | Kcnk3 | 0.020470418 |
| Glia_1 | Cml2 | 0.02234017 |
| Glia_1 | Ccdc24 | 0.026306226 |
| Glia_1 | Hs3st1 | 0.028130595 |
| Glia_1 | Dok7 | 0.029724311 |
| Glia_1 | Angpt4 | 0.029925287 |
| Glia_1 | Sec1 | 0.030421564 |
| Glia_1 | Dmrtb1 | 0.030577875 |
| Glia_1 | Prss35 | 0.030868418 |
| Glia_1 | E130018N17Rik | 0.031438115 |
| Glia_1 | E2f2 | 0.031806638 |
| Glia_1 | 1700101E01Rik | 0.032871926 |
| Glia_1 | 4930429F24Rik | 0.034754088 |
| Glia_1 | Cxcl14 | 0.037257002 |
| Glia_1 | Dll1 | 0.037594566 |
| Glia_1 | Lmo1 | 0.042079618 |
| Glia_1 | Arl9 | 0.042404944 |
| Glia_1 | Lax1 | 0.044533461 |
| Glia_1 | 9930014A18Rik | 0.049042233 |
| Glia_2 | Rgs6 | 2.9875E−108 |
| Glia_2 | Col28a1 | 3.24732E−80 |
| Glia_2 | Cadm2 | 2.49772E−79 |
| Glia_2 | Nrxn1 | 9.08305E−77 |
| Glia_2 | Xkr4 | 3.00526E−73 |
| Glia_2 | Scn7a | 2.95963E−66 |
| Glia_2 | Maml3 | 6.37856E−62 |
| Glia_2 | Ptprm | 7.36693E−54 |
| Glia_2 | Insc | 1.44019E−52 |
| Glia_2 | Piezo2 | 1.7486E−50 |
| Glia_2 | Ank3 | 2.03318E−49 |
| Glia_2 | Nav2 | 2.41183E−42 |
| Glia_2 | Ephb2 | 1.02252E−40 |
| Glia_2 | Rasgef1c | 3.627E−40 |
| Glia_2 | Rimklb | 3.07753E−39 |
| Glia_2 | Prnp | 3.91321E−38 |
| Glia_2 | Chst15 | 3.72061E−36 |
| Glia_2 | Col14a1 | 1.89348E−35 |
| Glia_2 | Col5a3 | 6.70987E−35 |
| Glia_2 | Lcp2 | 1.61702E−32 |
| Glia_2 | Lama2 | 3.91981E−31 |
| Glia_2 | Igsf21 | 1.34615E−29 |
| Glia_2 | Il34 | 3.05248E−29 |
| Glia_2 | Abca8b | 1.94271E−28 |
| Glia_2 | Ajap1 | 2.97444E−28 |
| Glia_2 | Klhl29 | 6.34965E−27 |
| Glia_2 | Grik2 | 1.76518E−26 |
| Glia_2 | Olfm2 | 2.22333E−26 |
| Glia_2 | Prex2 | 4.46867E−26 |
| Glia_2 | Kcna1 | 9.30801E−26 |
| Glia_2 | Tanc2 | 4.41436E−24 |
| Glia_2 | Slc35f1 | 6.22357E−24 |
| Glia_2 | Clvs1 | 1.03869E−23 |
| Glia_2 | Kank4 | 2.19424E−23 |
| Glia_2 | Frmd4a | 5.94549E−23 |
| Glia_2 | Gpnmb | 8.07252E−23 |
| Glia_2 | Matn2 | 1.63663E−22 |
| Glia_2 | Nkain2 | 1.64553E−21 |
| Glia_2 | Sgcd | 2.31678E−21 |
| Glia_2 | Deptor | 2.56698E−21 |
| Glia_2 | Dock11 | 2.95713E−21 |
| Glia_2 | Lphn3 | 3.65057E−20 |
| Glia_2 | Arpc1b | 4.64575E−20 |
| Glia_2 | Kcnip1 | 8.01507E−20 |
| Glia_2 | Wbscr17 | 2.61792E−19 |
| Glia_2 | Sfrp5 | 5.57014E−19 |
| Glia_2 | Hspg2 | 9.20002E−18 |
| Glia_2 | Mmp17 | 1.08463E−16 |
| Glia_2 | Kcnn2 | 2.29753E−16 |
| Glia_2 | Abca8a | 2.35532E−16 |
| Glia_2 | Slc22a23 | 2.9752E−16 |
| Glia_2 | Cspg4 | 2.66749E−15 |
| Glia_2 | Kcnk5 | 2.73549E−15 |
| Glia_2 | Gfra2 | 3.36902E−15 |
| Glia_2 | Ncam1 | 5.75721E−15 |
| Glia_2 | Cryab | 7.93853E−15 |
| Glia_2 | Kcna2 | 9.83328E−15 |
| Glia_2 | Gas2l3 | 9.83328E−15 |
| Glia_2 | Fbln1 | 1.27659E−14 |
| Glia_2 | Pmp22 | 1.64665E−14 |
| Glia_2 | Col5a1 | 2.01238E−14 |
| Glia_2 | Il16 | 5.3736E−14 |
| Glia_2 | Artn | 6.24451E−14 |
| Glia_2 | Adamts2 | 2.05954E−13 |
| Glia_2 | Ablim1 | 3.47608E−13 |
| Glia_2 | Pex5l | 3.57475E−13 |
| Glia_2 | Lbp | 8.59133E−13 |
| Glia_2 | C4b | 9.02476E−13 |
| Glia_2 | Epb4.1l4b | 1.30533E−12 |
| Glia_2 | Gli2 | 1.30533E−12 |
| Glia_2 | Prkca | 1.72494E−12 |
| Glia_2 | Nkd2 | 1.90045E−12 |
| Glia_2 | Sfrp1 | 2.23171E−12 |
| Glia_2 | Gulp1 | 3.81131E−12 |
| Glia_2 | Ngf | 3.93428E−12 |
| Glia_2 | Stard13 | 4.84026E−12 |
| Glia_2 | Cdh19 | 7.54394E−12 |
| Glia_2 | Pnpla3 | 9.29113E−12 |
| Glia_2 | L1cam | 9.45143E−12 |
| Glia_2 | Pdgfa | 1.73064E−11 |
| Glia_2 | Rnd3 | 3.67316E−11 |
| Glia_2 | Gas7 | 3.99808E−11 |
| Glia_2 | Vgll3 | 4.01943E−11 |
| Glia_2 | Art3 | 6.24539E−11 |
| Glia_2 | Nrp1 | 1.01557E−10 |
| Glia_2 | Mpp7 | 1.80742E−10 |
| Glia_2 | Adam12 | 2.59102E−10 |
| Glia_2 | Gm10863 | 3.82453E−10 |
| Glia_2 | Agap1 | 4.37285E−10 |
| Glia_2 | Synpo | 4.97644E−10 |
| Glia_2 | Arhgap39 | 8.33233E−10 |
| Glia_2 | Aspa | 9.03857E−10 |
| Glia_2 | Msrb3 | 1.48556E−09 |
| Glia_2 | 2610203C20Rik | 1.56141E−09 |
| Glia_2 | Arhgef37 | 1.57435E−09 |
| Glia_2 | Adamts12 | 1.62534E−09 |
| Glia_2 | Lypla2 | 2.27939E−09 |
| Glia_2 | Ivns1abp | 2.47973E−09 |
| Glia_2 | Nox4 | 2.55493E−09 |
| Glia_2 | Fgl2 | 2.57842E−09 |
| Glia_2 | Lamb1 | 8.82846E−09 |
| Glia_2 | Cdc42ep3 | 9.95911E−08 |
| Glia_2 | Tnfrsf1b | 1.54571E−07 |
| Glia_2 | Cmklr1 | 1.63653E−06 |
| Glia_2 | 1700010K23Rik | 1.72005E−06 |
| Glia_2 | Gbp10 | 1.56425E−05 |
| Glia_2 | Npc1l1 | 5.38733E−05 |
| Glia_2 | Mfap5 | 5.503E−05 |
| Glia_2 | Fam26e | 0.000131791 |
| Glia_2 | Mad2l1bp | 0.000228031 |
| Glia_2 | Cpn2 | 0.000394738 |
| Glia_2 | Rab37 | 0.000476322 |
| Glia_2 | Vmn2r84 | 0.000522186 |
| Glia_2 | Ssx9 | 0.000631415 |
| Glia_2 | A730085A09Rik | 0.000874202 |
| Glia_2 | A930016O22Rik | 0.000874202 |
| Glia_2 | Gm6249 | 0.000910352 |
| Glia_2 | Rfc4 | 0.001015805 |
| Glia_2 | 4930448I18Rik | 0.001044069 |
| Glia_2 | Gm20757 | 0.001047842 |
| Glia_2 | Hbegf | 0.001194231 |
| Glia_2 | Gm9920 | 0.001657125 |
| Glia_2 | 9430037G07Rik | 0.002144316 |
| Glia_2 | Gpr153 | 0.002488871 |
| Glia_2 | Gm3279 | 0.002519058 |
| Glia_2 | Krtap1-3 | 0.002551787 |
| Glia_2 | Nkx2-2as | 0.002582022 |
| Glia_2 | Colq | 0.002867618 |
| Glia_2 | Irs3 | 0.003182844 |
| Glia_2 | Cdc6 | 0.004473539 |
| Glia_2 | Apol9a | 0.004549637 |
| Glia_2 | Nlrp3 | 0.004676623 |
| Glia_2 | Mybpc3 | 0.004986801 |
| Glia_2 | Pde6c | 0.00509867 |
| Glia_2 | Hist1h2bh | 0.005429391 |
| Glia_2 | BC021891 | 0.005518423 |
| Glia_2 | 1700085C21Rik | 0.006436227 |
| Glia_2 | Arl4d | 0.006514074 |
| Glia_2 | Xkr7 | 0.007148345 |
| Glia_2 | Arr3 | 0.008118789 |
| Glia_2 | Nodal | 0.008473808 |
| Glia_2 | Rab27b | 0.010415343 |
| Glia_2 | Lrrn4cl | 0.011769879 |
| Glia_2 | Apol8 | 0.011827463 |
| Glia_2 | Mbnl3 | 0.01218435 |
| Glia_2 | Apol7a | 0.012969039 |
| Glia_2 | Gm10584 | 0.012985431 |
| Glia_2 | 5430440P10Rik | 0.013325935 |
| Glia_2 | Pde4c | 0.013760433 |
| Glia_2 | 9130227L01Rik | 0.014192439 |
| Glia_2 | Selplg | 0.01462793 |
| Glia_2 | Six4 | 0.016210118 |
| Glia_2 | Rnf43 | 0.016620049 |
| Glia_2 | Ninj2 | 0.016658095 |
| Glia_2 | P2ry10 | 0.019260995 |
| Glia_2 | Wdhd1 | 0.020242493 |
| Glia_2 | Gm13305 | 0.020463333 |
| Glia_2 | Gstm6 | 0.020684895 |
| Glia_2 | Mobp | 0.023679115 |
| Glia_2 | Rltpr | 0.024992858 |
| Glia_2 | Slc26a5 | 0.025230391 |
| Glia_2 | Nov | 0.026481593 |
| Glia_2 | Il5ra | 0.028377246 |
| Glia_2 | Gm8221 | 0.028978374 |
| Glia_2 | Sh2d1b2 | 0.03028365 |
| Glia_2 | B930025P03Rik | 0.03209964 |
| Glia_2 | Tubb1 | 0.03209964 |
| Glia_2 | Krtap1-4 | 0.032183185 |
| Glia_2 | Cacng5 | 0.033593585 |
| Glia_2 | Tmprss12 | 0.036709416 |
| Glia_2 | 4833427F10Rik | 0.041182614 |
| Glia_2 | Gm4858 | 0.044157528 |
| Glia_2 | Dbx2 | 0.046754332 |
| Glia_2 | Fam212a | 0.048170734 |
| Glia_2 | Selp | 0.048888788 |
| Glia_3 | Ntng1 | 3.1402E−121 |
| Glia_3 | Csmd1 | 3.35967E−75 |
| Glia_3 | Frmd4a | 2.14247E−66 |
| Glia_3 | Pappa | 1.66678E−65 |
| Glia_3 | Matn2 | 1.31184E−63 |
| Glia_3 | Slc2a13 | 6.75121E−63 |
| Glia_3 | Col6a3 | 1.39796E−61 |
| Glia_3 | Fndc1 | 6.27717E−61 |
| Glia_3 | Xylt1 | 1.96682E−60 |
| Glia_3 | Rasgef1c | 8.23307E−56 |
| Glia_3 | Cadm2 | 3.53989E−54 |
| Glia_3 | Kcna1 | 8.66125E−54 |
| Glia_3 | Specc1 | 2.86975E−45 |
| Glia_3 | Agap1 | 2.88939E−42 |
| Glia_3 | Scn7a | 1.21085E−40 |
| Glia_3 | Aspa | 7.70602E−39 |
| Glia_3 | Celf2 | 2.66842E−37 |
| Glia_3 | A330049N07Rik | 6.91802E−37 |
| Glia_3 | Nrp1 | 2.16648E−36 |
| Glia_3 | Col5a3 | 1.15592E−35 |
| Glia_3 | Epb4.1l4b | 1.25556E−35 |
| Glia_3 | Adam19 | 7.75581E−35 |
| Glia_3 | Ephb2 | 3.31129E−34 |
| Glia_3 | Prl3b1 | 5.05972E−34 |
| Glia_3 | Rcan2 | 2.84867E−33 |
| Glia_3 | Rxrg | 8.80895E−33 |
| Glia_3 | Mbp | 8.80895E−33 |
| Glia_3 | Ank3 | 9.0754E−32 |
| Glia_3 | Slc1a5 | 3.80479E−30 |
| Glia_3 | Kcnh8 | 7.57345E−30 |
| Glia_3 | Kcna2 | 4.08618E−28 |
| Glia_3 | Adamtsl1 | 4.29719E−28 |
| Glia_3 | Prex2 | 2.62789E−27 |
| Glia_3 | Antxr2 | 6.90021E−27 |
| Glia_3 | Itih5 | 2.45432E−26 |
| Glia_3 | Gfra3 | 2.55982E−26 |
| Glia_3 | Nkd2 | 3.34741E−26 |
| Glia_3 | Apba2 | 1.27407E−25 |
| Glia_3 | Ajap1 | 1.3498E−25 |
| Glia_3 | Clvs1 | 7.5139E−25 |
| Glia_3 | Col20a1 | 1.09261E−24 |
| Glia_3 | Sh3pxd2a | 1.30115E−24 |
| Glia_3 | Efemp1 | 1.75294E−24 |
| Glia_3 | Plxna2 | 2.7608E−24 |
| Glia_3 | Prima1 | 3.10485E−24 |
| Glia_3 | Kcnk13 | 1.41991E−23 |
| Glia_3 | Nav2 | 2.42242E−23 |
| Glia_3 | Col1a1 | 4.93057E−23 |
| Glia_3 | Prex1 | 8.43914E−23 |
| Glia_3 | Pde8a | 9.45957E−23 |
| Glia_3 | Postn | 1.23876E−22 |
| Glia_3 | Abca8b | 1.28381E−22 |
| Glia_3 | Epb4.1l3 | 1.65371E−21 |
| Glia_3 | Ppp1r12b | 4.27773E−21 |
| Glia_3 | Malat1 | 1.09631E−20 |
| Glia_3 | Vwa1 | 3.78833E−20 |
| Glia_3 | 1700047M11Rik | 3.93514E−20 |
| Glia_3 | Col28a1 | 4.51846E−20 |
| Glia_3 | Mpp7 | 6.26336E−20 |
| Glia_3 | Prnp | 9.00764E−20 |
| Glia_3 | Slc35f1 | 1.15646E−19 |
| Glia_3 | Iqgap2 | 1.23305E−19 |
| Glia_3 | Akap2 | 3.2087E−19 |
| Glia_3 | Nrn1 | 3.47583E−19 |
| Glia_3 | Sox6 | 6.14016E−19 |
| Glia_3 | Slc10a6 | 1.05524E−18 |
| Glia_3 | Insc | 1.32324E−18 |
| Glia_3 | Olfml2b | 1.54827E−18 |
| Glia_3 | Cpne8 | 1.9441E−18 |
| Glia_3 | Hspg2 | 5.38489E−18 |
| Glia_3 | Acsbg1 | 5.48077E−18 |
| Glia_3 | Srgap1 | 9.00375E−18 |
| Glia_3 | Col14a1 | 1.06822E−16 |
| Glia_3 | Neat1 | 1.16861E−16 |
| Glia_3 | Prkcq | 1.45074E−16 |
| Glia_3 | Adamts20 | 2.07065E−16 |
| Glia_3 | Nid2 | 2.08242E−16 |
| Glia_3 | Col1a2 | 2.10834E−16 |
| Glia_3 | Ppip5k2 | 5.34839E−16 |
| Glia_3 | Maml3 | 5.65913E−16 |
| Glia_3 | Ctnnd1 | 9.44667E−16 |
| Glia_3 | Gfra2 | 1.02206E−15 |
| Glia_3 | Cldn14 | 1.45265E−15 |
| Glia_3 | Stard13 | 1.75628E−15 |
| Glia_3 | Ndst3 | 1.84069E−15 |
| Glia_3 | Prickle2 | 2.2777E−15 |
| Glia_3 | Cspg4 | 2.84872E−15 |
| Glia_3 | Cables1 | 2.84872E−15 |
| Glia_3 | Zeb2 | 5.31706E−15 |
| Glia_3 | Lama2 | 6.0343E−15 |
| Glia_3 | Itpr2 | 1.37038E−14 |
| Glia_3 | Il1rap | 2.4431E−14 |
| Glia_3 | Cdk6 | 2.47593E−14 |
| Glia_3 | Ablim1 | 2.55903E−14 |
| Glia_3 | Sorcs2 | 2.55903E−14 |
| Glia_3 | Abca8a | 4.32193E−14 |
| Glia_3 | Adcy1 | 4.55758E−14 |
| Glia_3 | Gas2l3 | 6.006E−14 |
| Glia_3 | Lbh | 9.24711E−14 |
| Glia_3 | Art3 | 9.45182E−14 |
| Glia_3 | Dcpp3 | 2.15644E−13 |
| Glia_3 | Lypd6 | 8.27271E−13 |
| Glia_3 | Mlph | 7.78696E−12 |
| Glia_3 | D730001G18Rik | 9.14983E−12 |
| Glia_3 | Lgi1 | 2.82677E−11 |
| Glia_3 | 4932413F04Rik | 4.65592E−11 |
| Glia_3 | Mpz | 4.36451E−09 |
| Glia_3 | Rem1 | 5.12849E−09 |
| Glia_3 | Alx4 | 1.27083E−08 |
| Glia_3 | Klk8 | 3.1261E−07 |
| Glia_3 | Itga7 | 1.09984E−06 |
| Glia_3 | Klhl30 | 6.56023E−06 |
| Glia_3 | Tnfaip8l1 | 1.46027E−05 |
| Glia_3 | Ankrd53 | 2.63537E−05 |
| Glia_3 | Pygm | 3.05924E−05 |
| Glia_3 | Mb21d1 | 4.07807E−05 |
| Glia_3 | Trpc7 | 0.000134687 |
| Glia_3 | 4933431G14Rik | 0.000662086 |
| Glia_3 | Sema7a | 0.000907929 |
| Glia_3 | Dhrs9 | 0.000935153 |
| Glia_3 | Palmd | 0.000963442 |
| Glia_3 | Ina | 0.001456608 |
| Glia_3 | Neu2 | 0.001661338 |
| Glia_3 | Mtnr1a | 0.001806038 |
| Glia_3 | Klk10 | 0.001864488 |
| Glia_3 | Gcm2 | 0.002084779 |
| Glia_3 | Ces2b | 0.00214981 |
| Glia_3 | Slc22a14 | 0.002387257 |
| Glia_3 | Tdrd1 | 0.002500184 |
| Glia_3 | Gata5 | 0.003294594 |
| Glia_3 | Gm20187 | 0.003363837 |
| Glia_3 | Adra1d | 0.003775315 |
| Glia_3 | Gm19510 | 0.003939477 |
| Glia_3 | Smpdl3b | 0.004356848 |
| Glia_3 | Optc | 0.004782232 |
| Glia_3 | Gli1 | 0.005344248 |
| Glia_3 | Klk9 | 0.005508542 |
| Glia_3 | Ces2g | 0.006395829 |
| Glia_3 | Clec4d | 0.006412059 |
| Glia_3 | Zfp663 | 0.006460574 |
| Glia_3 | Il10 | 0.006817796 |
| Glia_3 | Ltk | 0.007065998 |
| Glia_3 | Gm10415 | 0.007328322 |
| Glia_3 | Htr3b | 0.009697209 |
| Glia_3 | Cyp4x1 | 0.0100129 |
| Glia_3 | Pnliprp2 | 0.010334957 |
| Glia_3 | 4933433F19Rik | 0.010539214 |
| Glia_3 | Asb4 | 0.011150368 |
| Glia_3 | Gm13582 | 0.012941176 |
| Glia_3 | Gm6121 | 0.013812083 |
| Glia_3 | Cacng2 | 0.014435786 |
| Glia_3 | Pax4 | 0.014711407 |
| Glia_3 | Adra1b | 0.014738884 |
| Glia_3 | Olfr389 | 0.015461699 |
| Glia_3 | Cd3e | 0.017181541 |
| Glia_3 | Psg19 | 0.017349299 |
| Glia_3 | Chrna2 | 0.017619426 |
| Glia_3 | Btnl2 | 0.018860223 |
| Glia_3 | Cyp2b23 | 0.018875949 |
| Glia_3 | Aqp2 | 0.020225909 |
| Glia_3 | Idi2 | 0.021844847 |
| Glia_3 | E230025N22Rik | 0.02402981 |
| Glia_3 | Gm13031 | 0.024452234 |
| Glia_3 | Tmprss7 | 0.025664004 |
| Glia_3 | 4930423M02Rik | 0.025664004 |
| Glia_3 | Pou3f1 | 0.026193435 |
| Glia_3 | Fst | 0.026801339 |
| Glia_3 | 1700034K08Rik | 0.027502267 |
| Glia_3 | Zfp541 | 0.028121716 |
| Glia_3 | F730043M19Rik | 0.030445926 |
| Glia_3 | Fam105a | 0.030530011 |
| Glia_3 | 4930461G14Rik | 0.031513524 |
| Glia_3 | Serpinb9e | 0.033724623 |
| Glia_3 | Adamts15 | 0.035299242 |
| Glia_3 | Osr1 | 0.037681426 |
| Glia_3 | Gm6792 | 0.03845916 |
| Glia_3 | Klra4 | 0.038744889 |
| Glia_3 | Grem2 | 0.039802663 |
| Glia_3 | Cts3 | 0.039979328 |
| Glia_3 | Scara3 | 0.039979328 |
| Glia_3 | Gm14483 | 0.040199595 |
| Glia_3 | D030025P21Rik | 0.040949965 |
| Glia_3 | Tmem40 | 0.046945019 |
| Glia_3 | Ptk6 | 0.049311297 |

TABLE 17

| ident | gene | padjH |
|---|---|---|
| Colonocytes | Cyp2c55 | 0.00E+00 |
| Colonocytes | Slc26a3 | 3.04E−253 |
| Colonocytes | Emp1 | 2.03E−182 |
| Colonocytes | Ceacam20 | 3.04E−182 |
| Colonocytes | Krt20 | 4.79E−177 |
| Colonocytes | Mxd1 | 2.90E−171 |
| Colonocytes | Lypd8 | 3.35E−170 |
| Colonocytes | Slc9a3 | 3.12E−166 |
| Colonocytes | Abcb1a | 4.06E−160 |
| Colonocytes | Atp2b1 | 2.16E−158 |
| Colonocytes | Lmo7 | 8.86E−157 |
| Colonocytes | Clec2h | 7.11E−146 |
| Colonocytes | Eps8 | 8.70E−146 |
| Colonocytes | Erbb2ip | 1.71E−143 |
| Colonocytes | Lgals3 | 3.90E−141 |
| Colonocytes | Muc3 | 4.88E−140 |
| Colonocytes | Maoa | 6.14E−140 |
| Colonocytes | Mgat4c | 1.17E−134 |
| Colonocytes | Ptprh | 2.32E−131 |
| Colonocytes | Cyp3a13 | 1.78E−130 |
| Colonocytes | Slc9a2 | 1.54E−128 |
| Colonocytes | Prom1 | 2.11E−125 |
| Colonocytes | Eps8l2 | 2.31E−125 |
| Colonocytes | Klf6 | 3.72E−125 |
| Colonocytes | Hsd3b3 | 4.67E−125 |
| Colonocytes | Clca4a | 2.01E−124 |
| Colonocytes | Prss30 | 4.51E−124 |
| Colonocytes | Myh14 | 5.13E−124 |
| Colonocytes | Slc8a1 | 6.63E−123 |
| Colonocytes | Ceacam1 | 6.63E−123 |
| Colonocytes | Stk25 | 1.28E−121 |
| Colonocytes | Myo15b | 4.10E−121 |
| Colonocytes | Iqgap2 | 3.46E−116 |
| Colonocytes | Coro2a | 1.49E−115 |
| Colonocytes | Guca2a | 2.57E−113 |
| Colonocytes | Ethe1 | 2.78E−113 |
| Colonocytes | Slc13a2 | 4.49E−109 |
| Colonocytes | Trpm6 | 3.24E−108 |
| Colonocytes | Fa2h | 5.67E−108 |
| Colonocytes | 2200002D01Rik | 2.18E−105 |
| Colonocytes | Pmp22 | 5.43E−105 |
| Colonocytes | Slc25a20 | 2.74E−99 |
| Colonocytes | Pls1 | 1.66E−98 |
| Colonocytes | Phgr1 | 1.99E−98 |
| Colonocytes | Ipmk | 3.46E−98 |
| Colonocytes | Sptssb | 1.04E−97 |
| Colonocytes | Nr3c2 | 2.36E−97 |
| Colonocytes | Klf4 | 5.36E−96 |
| Colonocytes | 1810065E05Rik | 3.26E−94 |
| Colonocytes | Areg | 1.17E−93 |
| Colonocytes | Tcf7l2 | 2.72E−93 |
| Colonocytes | St3gal4 | 5.90E−92 |
| Colonocytes | Akap13 | 7.96E−92 |
| Colonocytes | Nostrin | 1.24E−91 |
| Colonocytes | Sgk1 | 3.53E−91 |
| Colonocytes | Dsg2 | 6.04E−90 |
| Colonocytes | Chp1 | 1.25E−89 |
| Colonocytes | Misp | 3.31E−88 |
| Colonocytes | March3 | 2.48E−86 |
| Colonocytes | Gtpbp2 | 8.19E−86 |
| Colonocytes | 2010109I03Rik | 1.54E−85 |
| Colonocytes | Krt8 | 3.79E−85 |
| Colonocytes | Plac8 | 1.83E−83 |
| Colonocytes | Asap1 | 2.89E−83 |
| Colonocytes | Errfi1 | 1.35E−81 |
| Colonocytes | Nlrp9b | 3.24E−81 |
| Colonocytes | Selenbp1 | 1.36E−80 |
| Colonocytes | Pcsk5 | 1.37E−80 |
| Colonocytes | Car4 | 4.58E−80 |
| Colonocytes | Ezr | 5.89E−80 |
| Colonocytes | Sema3c | 6.22E−80 |
| Colonocytes | Ms4a8a | 2.76E−79 |
| Colonocytes | Gda | 2.97E−79 |
| Colonocytes | Pla2g3 | 5.31E−79 |
| Colonocytes | Usp53 | 1.49E−77 |
| Colonocytes | Specc1l | 2.84E−77 |
| Colonocytes | Nudt4 | 4.14E−77 |
| Colonocytes | Pparg | 2.79E−76 |
| Colonocytes | Higd1a | 4.05E−75 |
| Colonocytes | Atp10b | 5.91E−75 |
| Colonocytes | Abcg2 | 1.90E−73 |
| Colonocytes | Myo1e | 3.73E−73 |
| Colonocytes | Actn4 | 4.55E−73 |
| Colonocytes | Gprc5a | 4.68E−73 |
| Colonocytes | Rnasel | 2.27E−72 |
| Colonocytes | Aqp8 | 4.11E−72 |
| Colonocytes | Car1 | 5.06E−72 |
| Colonocytes | Cdkn1a | 5.44E−72 |
| Colonocytes | Cdhr5 | 1.23E−70 |
| Colonocytes | Muc13 | 1.23E−70 |
| Colonocytes | Mep1b | 2.30E−70 |
| Colonocytes | Xist | 3.72E−70 |
| Colonocytes | Ces2a | 4.74E−70 |
| Colonocytes | Ugdh | 3.64E−69 |
| Colonocytes | Rock2 | 1.57E−68 |
| Colonocytes | Stk10 | 1.13E−67 |
| Colonocytes | Cyp2d34 | 4.86E−67 |
| Colonocytes | Apol10a | 8.61E−67 |
| Colonocytes | Prdx6 | 9.14E−67 |
| Colonocytes | Slc13a1 | 2.35E−66 |
| Colonocytes | Noct | 1.60E−64 |
| Colonocytes | 4732465J04Rik | 1.77E−64 |
| Colonocytes | Sgk2 | 3.93E−64 |
| Colonocytes | Lama3 | 6.28E−64 |
| Colonocytes | Ccng2 | 1.17E−63 |
| Colonocytes | Sdcbp2 | 4.30E−62 |
| Colonocytes | Cpn1 | 4.42E−60 |
| Colonocytes | Tmigd1 | 1.39E−59 |
| Colonocytes | Aldh1a1 | 3.18E−52 |
| Colonocytes | 4930539E08Rik | 1.43E−50 |
| Colonocytes | Hbegf | 2.24E−50 |
| Colonocytes | Abca12 | 4.79E−48 |
| Colonocytes | Gm3054 | 1.57E−45 |
| Colonocytes | Gm15345 | 3.18E−44 |
| Colonocytes | Cry1 | 2.59E−43 |
| Colonocytes | Dhrs9 | 1.31E−42 |
| Colonocytes | Cyp4f14 | 6.42E−42 |
| Colonocytes | Rhod | 3.13E−38 |
| Colonocytes | Tmem140 | 5.09E−37 |
| Colonocytes | Slc10a2 | 8.04E−37 |
| Colonocytes | 5430427M07Rik | 1.15E−36 |
| Colonocytes | Ttc22 | 4.25E−36 |
| Colonocytes | Ppm1j | 2.01E−34 |
| Colonocytes | Fmo5 | 3.40E−34 |
| Colonocytes | Lamb3 | 1.05E−33 |
| Colonocytes | Mal | 1.91E−33 |
| Colonocytes | Tmem252 | 5.05E−32 |
| Colonocytes | Dgat2 | 8.80E−31 |
| Colonocytes | Anks4b | 1.67E−30 |
| Colonocytes | Dusp10 | 3.49E−30 |
| Colonocytes | RP23-143J24.4 | 5.89E−30 |
| Colonocytes | Hkdc1 | 7.51E−30 |
| Colonocytes | Ptk6 | 1.83E−29 |
| Colonocytes | Cidec | 2.45E−29 |
| Colonocytes | Igsf9 | 1.96E−28 |
| Colonocytes | Ifit1bl1 | 2.60E−27 |
| Colonocytes | Atf3 | 4.07E−27 |
| Colonocytes | H2-Q1 | 5.42E−27 |
| Colonocytes | Trim40 | 1.05E−25 |
| Colonocytes | Sult2b1 | 1.28E−25 |
| Colonocytes | Gm15998 | 4.99E−24 |
| Colonocytes | Syt12 | 8.81E−24 |
| Colonocytes | Chp2 | 1.49E−23 |
| Colonocytes | 3100003L05Rik | 1.97E−23 |
| Colonocytes | Trim15 | 4.75E−23 |
| Colonocytes | Slc3a1 | 2.17E−22 |
| Colonocytes | Plekhg6 | 6.26E−22 |
| Colonocytes | Gm16233 | 7.02E−21 |
| Colonocytes | Slc51b | 2.40E−20 |
| Colonocytes | Sh3tc1 | 8.35E−20 |
| Colonocytes | Agbl2 | 2.06E−17 |
| Colonocytes | Grin3a | 3.32E−17 |
| Colonocytes | Slc34a2 | 9.50E−17 |
| Colonocytes | Cxcl16 | 5.07E−16 |
| Colonocytes | Bmp8b | 2.09E−15 |
| Colonocytes | C130074G19Rik | 3.61E−15 |
| Colonocytes | Sprr1a | 7.30E−15 |
| Colonocytes | Maff | 2.31E−14 |
| Colonocytes | Adamts18 | 2.84E−14 |
| Colonocytes | Oasl1 | 3.40E−14 |
| Colonocytes | Tat | 4.38E−14 |
| Colonocytes | Psg28 | 1.39E−13 |
| Colonocytes | Akr1b7 | 1.59E−13 |
| Colonocytes | E130012A19Rik | 2.25E−13 |
| Colonocytes | Aspa | 2.25E−13 |
| Colonocytes | Baat | 4.58E−13 |
| Colonocytes | Arg2 | 5.32E−13 |
| Colonocytes | Rsad2 | 1.19E−12 |
| Colonocytes | Ifit1bl2 | 6.99E−12 |
| Colonocytes | 2010005H15Rik | 1.60E−11 |
| Colonocytes | Gm10522 | 6.65E−11 |
| Colonocytes | Slc30a10 | 2.66E−09 |
| Colonocytes | Myom3 | 6.15E−09 |
| Colonocytes.1 | Prdx6 | 4.33E−223 |
| Colonocytes.1 | Lypd8 | 1.89E−199 |
| Colonocytes.1 | Tgm3 | 4.84E−179 |
| Colonocytes.1 | Car4 | 4.08E−166 |
| Colonocytes.1 | Slc26a3 | 5.20E−154 |
| Colonocytes.1 | Saa1 | 1.62E−153 |
| Colonocytes.1 | Ceacam20 | 3.54E−146 |
| Colonocytes.1 | Slc20a1 | 2.83E−142 |
| Colonocytes.1 | Sepp1 | 1.06E−138 |
| Colonocytes.1 | Muc3 | 1.24E−136 |
| Colonocytes.1 | Slc15a1 | 2.15E−129 |
| Colonocytes.1 | Fxyd4 | 4.27E−121 |
| Colonocytes.1 | Slc37a2 | 4.91E−118 |
| Colonocytes.1 | Sprr2a3 | 1.20E−117 |
| Colonocytes.1 | Aqp8 | 1.75E−116 |
| Colonocytes.1 | Atp12a | 2.75E−116 |
| Colonocytes.1 | Tmem45b | 3.13E−115 |
| Colonocytes.1 | Mxd1 | 6.43E−115 |
| Colonocytes.1 | Crip1 | 1.09E−109 |
| Colonocytes.1 | Trpm6 | 2.21E−109 |
| Colonocytes.1 | Plac8 | 6.62E−107 |
| Colonocytes.1 | Rdh16 | 1.87E−106 |
| Colonocytes.1 | Cyp2c55 | 5.20E−105 |
| Colonocytes.1 | Ggh | 1.18E−101 |
| Colonocytes.1 | Clic5 | 9.32E−101 |
| Colonocytes.1 | 2200002D01Rik | 1.06E−100 |
| Colonocytes.1 | Slc26a2 | 6.04E−97 |
| Colonocytes.1 | Cyp2d34 | 1.24E−94 |
| Colonocytes.1 | Abcb1a | 3.61E−94 |
| Colonocytes.1 | Tnni1 | 1.49E−93 |
| Colonocytes.1 | Ly6g | 6.71E−91 |
| Colonocytes.1 | Gpr137b | 9.44E−90 |
| Colonocytes.1 | Slc6a8 | 3.39E−87 |
| Colonocytes.1 | Phlpp2 | 7.14E−87 |
| Colonocytes.1 | AA467197 | 1.59E−86 |
| Colonocytes.1 | Slc40a1 | 2.11E−86 |
| Colonocytes.1 | Btg1 | 4.24E−86 |
| Colonocytes.1 | Mgat4a | 2.84E−84 |
| Colonocytes.1 | Ifit1bl1 | 3.57E−84 |
| Colonocytes.1 | Sectm1b | 4.20E−82 |
| Colonocytes.1 | Tm4sf20 | 4.20E−82 |
| Colonocytes.1 | Sult1b1 | 1.71E−81 |
| Colonocytes.1 | B4galt1 | 1.38E−80 |
| Colonocytes.1 | Pmp22 | 2.32E−80 |
| Colonocytes.1 | Slc9a2 | 2.62E−80 |
| Colonocytes.1 | Sycn | 3.84E−79 |
| Colonocytes.1 | Nudt4 | 9.75E−79 |
| Colonocytes.1 | March3 | 7.99E−77 |
| Colonocytes.1 | Tspan1 | 8.80E−77 |
| Colonocytes.1 | Slco2a1 | 2.89E−76 |
| Colonocytes.1 | Endod1 | 2.89E−76 |
| Colonocytes.1 | Prss30 | 7.15E−76 |
| Colonocytes.1 | Myo15b | 2.53E−75 |
| Colonocytes.1 | Tns4 | 9.42E−75 |
| Colonocytes.1 | Fth1 | 1.62E−74 |
| Colonocytes.1 | Fbxo32 | 2.08E−74 |
| Colonocytes.1 | Atp2b1 | 1.92E−73 |
| Colonocytes.1 | Nlrp9b | 5.95E−73 |
| Colonocytes.1 | Nt5e | 6.93E−73 |
| Colonocytes.1 | Atp1b1 | 3.20E−72 |
| Colonocytes.1 | Coro2a | 1.47E−71 |
| Colonocytes.1 | Sprr2a2 | 6.53E−71 |
| Colonocytes.1 | 2610528A11Rik | 2.00E−70 |
| Colonocytes.1 | Cnnm4 | 6.84E−70 |
| Colonocytes.1 | St3gal4 | 9.77E−70 |
| Colonocytes.1 | Ipmk | 4.63E−69 |
| Colonocytes.1 | Lama3 | 8.12E−69 |
| Colonocytes.1 | Ahnak | 8.98E−69 |
| Colonocytes.1 | Max | 1.20E−68 |
| Colonocytes.1 | Ctss | 1.88E−68 |
| Colonocytes.1 | Ly6a | 2.83E−68 |
| Colonocytes.1 | 9530026P05Rik | 4.21E−68 |
| Colonocytes.1 | Mep1a | 4.72E−68 |
| Colonocytes.1 | Myl12b | 5.18E−68 |
| Colonocytes.1 | Bmp2 | 8.43E−68 |
| Colonocytes.1 | Cs | 1.11E−67 |
| Colonocytes.1 | Myh14 | 2.11E−67 |
| Colonocytes.1 | Krt20 | 3.83E−67 |
| Colonocytes.1 | Eif2s2 | 5.01E−66 |
| Colonocytes.1 | Themis3 | 9.17E−66 |
| Colonocytes.1 | Abat | 2.49E−65 |
| Colonocytes.1 | Ifngr1 | 3.26E−65 |
| Colonocytes.1 | Misp | 3.44E−65 |
| Colonocytes.1 | mt-Co1 | 5.57E−65 |
| Colonocytes.1 | Itm2b | 9.06E−65 |
| Colonocytes.1 | Itih5 | 2.11E−64 |
| Colonocytes.1 | Slc25a20 | 3.39E−64 |
| Colonocytes.1 | Pdzk1ip1 | 4.76E−63 |
| Colonocytes.1 | Rhoc | 7.42E−63 |
| Colonocytes.1 | S100a6 | 1.32E−62 |
| Colonocytes.1 | Ckmt1 | 1.83E−62 |
| Colonocytes.1 | Rhbdl2 | 3.01E−62 |
| Colonocytes.1 | Gm30613 | 3.16E−62 |
| Colonocytes.1 | Prr15l | 4.47E−62 |
| Colonocytes.1 | Mcl1 | 6.59E−62 |
| Colonocytes.1 | Noct | 2.53E−61 |
| Colonocytes.1 | Fhl1 | 4.07E−61 |
| Colonocytes.1 | mt-Atp6 | 4.74E−61 |
| Colonocytes.1 | Tmem37 | 1.76E−60 |
| Colonocytes.1 | Prss32 | 2.31E−60 |
| Colonocytes.1 | Unc119 | 4.09E−56 |
| Colonocytes.1 | Pmaip1 | 4.07E−55 |
| Colonocytes.1 | Gcnt1 | 9.48E−55 |
| Colonocytes.1 | Mt1 | 1.45E−54 |
| Colonocytes.1 | Lhfpl2 | 2.32E−51 |
| Colonocytes.1 | Pllp | 3.26E−51 |
| Colonocytes.1 | Gpr137b-ps | 2.31E−49 |
| Colonocytes.1 | Stom | 3.20E−49 |
| Colonocytes.1 | Trpv3 | 1.59E−48 |
| Colonocytes.1 | 2310079G19Rik | 1.72E−48 |
| Colonocytes.1 | Tmem140 | 1.22E−46 |
| Colonocytes.1 | Apob | 6.68E−45 |
| Colonocytes.1 | Rbp2 | 1.18E−43 |
| Colonocytes.1 | Otub1 | 2.25E−41 |
| Colonocytes.1 | A930011G23Rik | 4.19E−39 |
| Colonocytes.1 | Upp1 | 8.72E−38 |
| Colonocytes.1 | Slc34a2 | 1.01E−35 |
| Colonocytes.1 | H2-Q1 | 1.99E−35 |
| Colonocytes.1 | Cyp2d12 | 2.83E−35 |
| Colonocytes.1 | Eno3 | 3.02E−33 |
| Colonocytes.1 | Edn1 | 6.17E−32 |
| Colonocytes.1 | Tnfaip3 | 5.90E−30 |
| Colonocytes.1 | Slc30a10 | 1.23E−29 |
| Colonocytes.1 | Cyp2d10 | 7.41E−28 |
| Colonocytes.1 | Slc30a10 | 1.89E−27 |
| Colonocytes.1 | Pla2g4f | 6.22E−27 |
| Colonocytes.1 | Gm15998 | 2.52E−26 |
| Colonocytes.1 | 4930552P12Rik | 3.32E−26 |
| Colonocytes.1 | Hoxd11 | 2.67E−23 |
| Colonocytes.1 | Abcg8 | 1.16E−22 |
| Colonocytes.1 | 2010003K11Rik | 1.53E−22 |
| Colonocytes.1 | Gjb5 | 3.49E−21 |
| Colonocytes.1 | Gm31363 | 1.35E−20 |
| Colonocytes.1 | 4833407H14Rik | 1.68E−20 |
| Colonocytes.1 | D330045A20Rik | 2.32E−20 |
| Colonocytes.1 | Slc46a1 | 1.88E−19 |
| Colonocytes.1 | Anxa8 | 1.28E−18 |
| Colonocytes.1 | Prdx6b | 2.08E−18 |
| Colonocytes.1 | Abcg5 | 2.51E−18 |
| Colonocytes.1 | Hist1h4h | 3.93E−18 |
| Colonocytes.1 | Asb11 | 2.41E−17 |
| Colonocytes.1 | Cyp2d9 | 2.94E−17 |
| Colonocytes.1 | 2010005H15Rik | 1.22E−16 |
| Colonocytes.1 | Xkr9 | 6.56E−16 |
| Colonocytes.1 | S100g | 2.27E−15 |
| Colonocytes.1 | Gm13412 | 2.94E−15 |
| Colonocytes.1 | Hoxd13 | 1.17E−14 |
| Colonocytes.1 | Csta1 | 4.35E−14 |
| Colonocytes.1 | Zfp775 | 3.20E−13 |
| Colonocytes.1 | Trpv6 | 1.89E−12 |
| Colonocytes.1 | 1700019G17Rik | 2.65E−12 |
| Colonocytes.1 | Scnn1g | 4.23E−12 |
| Colonocytes.1 | Plekhg6 | 4.26E−12 |
| Colonocytes.1 | Abcg1 | 8.93E−12 |
| Colonocytes.1 | BC025446 | 1.25E−11 |
| Colonocytes.1 | Slc16a3 | 1.60E−10 |
| Colonocytes.1 | Ifit1bl2 | 4.83E−10 |
| Colonocytes.1 | Gm12056 | 7.25E−10 |
| Colonocytes.1 | Gm11535 | 9.59E−10 |
| Colonocytes.1 | Ttc39c | 1.96E−09 |
| Colonocytes.1 | Ceacam18 | 2.75E−09 |
| Colonocytes.1 | Saa2 | 3.09E−09 |
| Colonocytes.2 | mt-Co1 | 1.95E−188 |
| Colonocytes.2 | mt-Co3 | 7.32E−171 |
| Colonocytes.2 | Guca2a | 1.39E−161 |
| Colonocytes.2 | mt-Atp6 | 6.92E−155 |
| Colonocytes.2 | mt-Co2 | 9.78E−152 |
| Colonocytes.2 | mt-Nd1 | 7.29E−130 |
| Colonocytes.2 | Phgr1 | 1.33E−125 |
| Colonocytes.2 | 2200002D01Rik | 4.24E−116 |
| Colonocytes.2 | Muc3 | 1.22E−115 |
| Colonocytes.2 | mt-Cytb | 1.98E−111 |
| Colonocytes.2 | Cox8a | 8.93E−104 |
| Colonocytes.2 | Cyp2c55 | 5.95E−103 |
| Colonocytes.2 | mt-Nd4 | 5.35E−96 |
| Colonocytes.2 | S100a6 | 4.16E−88 |
| Colonocytes.2 | Cox6a1 | 9.10E−84 |
| Colonocytes.2 | Fth1 | 1.18E−83 |
| Colonocytes.2 | Cox6c | 3.06E−83 |
| Colonocytes.2 | Car1 | 9.22E−83 |
| Colonocytes.2 | Crip1 | 6.84E−75 |
| Colonocytes.2 | Lgals3 | 1.54E−73 |
| Colonocytes.2 | 1810065E05Rik | 1.00E−72 |
| Colonocytes.2 | Fabp2 | 1.12E−71 |
| Colonocytes.2 | Rplp1 | 2.08E−70 |
| Colonocytes.2 | Tmsb4x | 2.42E−70 |
| Colonocytes.2 | Emp1 | 3.75E−66 |
| Colonocytes.2 | Krt8 | 2.90E−65 |
| Colonocytes.2 | Cox7a2 | 5.20E−63 |
| Colonocytes.2 | Mgat4c | 9.61E−63 |
| Colonocytes.2 | Gm10073 | 3.78E−62 |
| Colonocytes.2 | Cox6b1 | 4.80E−62 |
| Colonocytes.2 | Chchd10 | 2.84E−60 |
| Colonocytes.2 | Slc6a14 | 2.52E−59 |
| Colonocytes.2 | Plac8 | 5.58E−59 |
| Colonocytes.2 | Slc16a1 | 2.77E−58 |
| Colonocytes.2 | mt-Nd2 | 2.77E−58 |
| Colonocytes.2 | Atp1a1 | 4.21E−58 |
| Colonocytes.2 | Lypd8 | 6.01E−57 |
| Colonocytes.2 | Aqp4 | 6.94E−56 |
| Colonocytes.2 | Slc26a3 | 1.86E−55 |
| Colonocytes.2 | Calm1 | 4.23E−55 |
| Colonocytes.2 | mt-Nd5 | 4.76E−55 |
| Colonocytes.2 | Uqcrh | 6.72E−55 |
| Colonocytes.2 | Uqcrq | 7.34E−55 |
| Colonocytes.2 | Uqcr11 | 2.94E−54 |
| Colonocytes.2 | Atp1b1 | 1.72E−53 |
| Colonocytes.2 | Slc26a2 | 2.86E−53 |
| Colonocytes.2 | Bsg | 1.11E−52 |
| Colonocytes.2 | Gm9843 | 2.43E−51 |
| Colonocytes.2 | Tmbim6 | 3.06E−51 |
| Colonocytes.2 | Ndufa6 | 4.18E−51 |
| Colonocytes.2 | Serf2 | 8.43E−50 |
| Colonocytes.2 | Ethe1 | 4.11E−49 |
| Colonocytes.2 | Atp5j2 | 4.82E−49 |
| Colonocytes.2 | Ftl1 | 1.11E−48 |
| Colonocytes.2 | Prdx6 | 3.29E−48 |
| Colonocytes.2 | Perp | 4.22E−48 |
| Colonocytes.2 | Ndufa1 | 5.01E−48 |
| Colonocytes.2 | Endod1 | 3.95E−47 |
| Colonocytes.2 | Cdhr5 | 4.68E−47 |
| Colonocytes.2 | Cox5a | 6.56E−47 |
| Colonocytes.2 | AA467197 | 7.59E−47 |
| Colonocytes.2 | Dmbt1 | 1.64E−46 |
| Colonocytes.2 | mt-Nd4l | 1.75E−46 |
| Colonocytes.2 | Selenbp1 | 2.10E−46 |
| Colonocytes.2 | 2010107E04Rik | 4.66E−46 |
| Colonocytes.2 | Apol10a | 1.39E−45 |
| Colonocytes.2 | Rfk | 2.51E−45 |
| Colonocytes.2 | Cox7c | 3.76E−45 |
| Colonocytes.2 | Uqcr10 | 6.31E−44 |
| Colonocytes.2 | Mkrn1 | 9.28E−44 |
| Colonocytes.2 | Abhd11os | 4.35E−43 |
| Colonocytes.2 | Edf1 | 1.05E−42 |
| Colonocytes.2 | Cnnm4 | 4.52E−42 |
| Colonocytes.2 | Cycs | 5.38E−42 |
| Colonocytes.2 | Mep1a | 6.86E−42 |
| Colonocytes.2 | Krt19 | 1.05E−41 |
| Colonocytes.2 | Atp5j | 1.14E−41 |
| Colonocytes.2 | Plec | 1.31E−41 |
| Colonocytes.2 | S100a10 | 4.41E−41 |
| Colonocytes.2 | Cox5b | 4.80E−41 |
| Colonocytes.2 | Ndufa2 | 5.23E−41 |
| Colonocytes.2 | Ndufb9 | 9.33E−41 |
| Colonocytes.2 | Cyp2c65 | 1.03E−40 |
| Colonocytes.2 | Atp5e | 2.87E−40 |
| Colonocytes.2 | Tgoln1 | 3.20E−40 |
| Colonocytes.2 | Epcam | 4.73E−40 |
| Colonocytes.2 | Cdh17 | 5.68E−40 |
| Colonocytes.2 | Minos1 | 5.94E−40 |
| Colonocytes.2 | Ceacam1 | 2.63E−39 |
| Colonocytes.2 | Krt20 | 4.49E−39 |
| Colonocytes.2 | Ahnak | 2.84E−38 |
| Colonocytes.2 | Spint2 | 4.08E−38 |
| Colonocytes.2 | Psap | 4.24E−38 |
| Colonocytes.2 | Gpx1 | 4.36E−38 |
| Colonocytes.2 | Tmsb10 | 4.87E−38 |
| Colonocytes.2 | Txn1 | 5.46E−38 |
| Colonocytes.2 | Mgst3 | 9.65E−38 |
| Colonocytes.2 | Cox7b | 1.09E−37 |
| Colonocytes.2 | Itm2b | 1.52E−37 |
| Colonocytes.2 | Tspan1 | 3.45E−37 |
| Colonocytes.2 | Ces2e | 5.43E−35 |
| Colonocytes.2 | Entpd5 | 7.35E−34 |
| Colonocytes.2 | Slc35g1 | 3.66E−32 |
| Colonocytes.2 | Cwh43 | 7.28E−31 |
| Colonocytes.2 | Mall | 4.17E−29 |
| Colonocytes.2 | Sptssb | 5.20E−29 |
| Colonocytes.2 | Smim24 | 9.42E−29 |
| Colonocytes.2 | Plpp2 | 1.14E−27 |
| Colonocytes.2 | Fzd5 | 1.39E−27 |
| Colonocytes.2 | Ndufa8 | 1.52E−27 |
| Colonocytes.2 | Gm3336 | 9.80E−27 |
| Colonocytes.2 | Slc27a4 | 1.70E−26 |
| Colonocytes.2 | Prdx5 | 1.91E−26 |
| Colonocytes.2 | Lad1 | 6.16E−26 |
| Colonocytes.2 | Cox7a1 | 1.22E−25 |
| Colonocytes.2 | 1810043H04Rik | 1.32E−25 |
| Colonocytes.2 | Nlrp4e | 5.08E−25 |
| Colonocytes.2 | Abcb6 | 5.63E−25 |
| Colonocytes.2 | Lipg | 2.92E−21 |
| Colonocytes.2 | Slc9a3r1 | 2.96E−21 |
| Colonocytes.2 | Erbb2 | 7.09E−21 |
| Colonocytes.2 | Ermp1 | 1.93E−19 |
| Colonocytes.2 | Cyp4f14 | 4.35E−19 |
| Colonocytes.2 | Apob | 7.24E−19 |
| Colonocytes.2 | H2-Q2 | 1.05E−18 |
| Colonocytes.2 | Fam83h | 3.46E−18 |
| Colonocytes.2 | Gm5617 | 3.16E−17 |
| Colonocytes.2 | Ube2m | 3.91E−17 |
| Colonocytes.2 | Prap1 | 1.18E−16 |
| Colonocytes.2 | Adra2a | 1.04E−15 |
| Colonocytes.2 | Rab8a | 3.37E−15 |
| Colonocytes.2 | Cyp2c69 | 4.20E−14 |
| Colonocytes.2 | Slc51b | 6.72E−14 |
| Colonocytes.2 | Tmem252 | 1.68E−13 |
| Colonocytes.2 | Srxn1 | 2.87E−13 |
| Colonocytes.2 | S100g | 6.70E−13 |
| Colonocytes.2 | Slc39a4 | 7.22E−13 |
| Colonocytes.2 | Tmigd1 | 3.34E−12 |
| Colonocytes.2 | Txnl4a | 4.23E−12 |
| Colonocytes.2 | Akr1b8 | 5.62E−12 |
| Colonocytes.2 | Edn2 | 6.33E−12 |
| Colonocytes.2 | Slc51a | 8.64E−12 |
| Colonocytes.2 | Aldob | 1.39E−11 |
| Colonocytes.2 | Ankrd50 | 7.05E−11 |
| Colonocytes.2 | Cda | 8.44E−11 |
| Colonocytes.2 | Slc22a19 | 1.05E−10 |
| Colonocytes.2 | Slc39a3 | 1.81E−10 |
| Colonocytes.2 | Slc30a1 | 3.43E−10 |
| Colonocytes.2 | Akr1c12 | 1.12E−09 |
| Colonocytes.2 | Ehd1 | 4.59E−09 |
| Colonocytes.2 | Gsta1 | 8.46E−09 |
| Colonocytes.2 | Rxra | 2.77E−08 |
| Colonocytes.2 | Hkdc1 | 5.17E−08 |
| Colonocytes.2 | mt-Nd6 | 2.24E−07 |
| Colonocytes.2 | Mal | 7.51E−07 |
| Colonocytes.2 | Ttll12 | 1.22E−06 |
| Colonocytes.2 | Gm44026 | 2.05E−06 |
| Colonocytes.2 | Hsd17b13 | 2.27E−06 |
| Colonocytes.2 | Chp2 | 4.56E−06 |
| Colonocytes.2 | Vps4a | 5.78E−06 |
| Colonocytes.2 | Fzd8 | 1.19E−05 |
| Colonocytes.2 | 2010003K11Rik | 3.31E−05 |
| Colonocytes.2 | Gm42562 | 2.80E−04 |
| Colonocytes.2 | Lsm14b | 2.80E−04 |
| Colonocytes.2 | Cdc42ep2 | 2.85E−04 |
| Colonocytes.2 | Rbp2 | 3.14E−03 |
| Endothelial | Mmrn1 | 6.76E−152 |
| Endothelial | Reln | 3.16E−148 |
| Endothelial | Ccl21a | 3.25E−125 |
| Endothelial | Nxn | 2.60E−117 |
| Endothelial | Galnt18 | 1.05E−109 |
| Endothelial | Ldb2 | 6.41E−108 |
| Endothelial | Lyve1 | 2.93E−103 |
| Endothelial | Prex2 | 2.22E−97 |
| Endothelial | Ebf1 | 2.17E−94 |
| Endothelial | Rbms1 | 3.54E−94 |
| Endothelial | Timp3 | 5.88E−91 |
| Endothelial | cp | 6.48E−87 |
| Endothelial | Cldn5 | 1.45E−85 |
| Endothelial | Pecam1 | 1.35E−79 |
| Endothelial | Fmnl2 | 1.35E−79 |
| Endothelial | Rhoj | 2.25E−78 |
| Endothelial | Abi3bp | 1.21E−77 |
| Endothelial | Pitpnc1 | 1.32E−76 |
| Endothelial | Kank3 | 2.95E−76 |
| Endothelial | Igfbp5 | 3.45E−74 |
| Endothelial | Fgl2 | 3.20E−71 |
| Endothelial | Sema6a | 5.21E−71 |
| Endothelial | Wdr17 | 7.84E−71 |
| Endothelial | Ntn1 | 1.18E−69 |
| Endothelial | Sptbn1 | 4.92E−69 |
| Endothelial | Podxl | 2.05E−67 |
| Endothelial | Wipf3 | 1.97E−65 |
| Endothelial | Elk3 | 7.59E−62 |
| Endothelial | Pard6g | 1.11E−61 |
| Endothelial | Lama4 | 1.91E−61 |
| Endothelial | Shank3 | 6.40E−61 |
| Endothelial | Tshz2 | 1.17E−59 |
| Endothelial | Sema3d | 1.60E−58 |
| Endothelial | Cyyr1 | 4.67E−58 |
| Endothelial | Dlg1 | 4.67E−58 |
| Endothelial | Flt4 | 1.49E−57 |
| Endothelial | Emcn | 3.49E−57 |
| Endothelial | Thsd7a | 2.49E−56 |
| Endothelial | Dock9 | 1.03E−55 |
| Endothelial | 4930448N21Rik | 2.05E−55 |
| Endothelial | Utrn | 2.95E−55 |
| Endothelial | Ptprm | 3.22E−54 |
| Endothelial | Ece1 | 4.01E−53 |
| Endothelial | Dock4 | 2.71E−52 |
| Endothelial | Tspan9 | 7.97E−52 |
| Endothelial | Piezo2 | 2.08E−51 |
| Endothelial | Zfpm2 | 5.82E−51 |
| Endothelial | Fgd5 | 9.17E−51 |
| Endothelial | D5Ertd615e | 4.61E−50 |
| Endothelial | Sdpr | 5.57E−50 |
| Endothelial | 9330175M20Rik | 6.46E−50 |
| Endothelial | Tll1 | 6.23E−49 |
| Endothelial | Adgrg3 | 6.44E−49 |
| Endothelial | Maf | 7.24E−49 |
| Endothelial | Etl4 | 9.60E−49 |
| Endothelial | Malat1 | 2.60E−47 |
| Endothelial | Calcrl | 3.63E−47 |
| Endothelial | Adgrl4 | 1.70E−46 |
| Endothelial | Prox1 | 1.81E−46 |
| Endothelial | Nfat5 | 1.96E−46 |
| Endothelial | Cped1 | 3.37E−46 |
| Endothelial | Gab2 | 4.41E−46 |
| Endothelial | Hspa12b | 8.92E−46 |
| Endothelial | Cav1 | 2.05E−45 |
| Endothelial | Prkg1 | 3.22E−43 |
| Endothelial | Gm2163 | 3.92E−43 |
| Endothelial | Tanc2 | 2.85E−42 |
| Endothelial | Tns1 | 3.06E−42 |
| Endothelial | Kalrn | 8.19E−42 |
| Endothelial | Meis2 | 9.32E−42 |
| Endothelial | Dennd4a | 1.97E−41 |
| Endothelial | Ppp1r2 | 2.42E−41 |
| Endothelial | Zfp521 | 3.09E−41 |
| Endothelial | Hip1 | 4.51E−41 |
| Endothelial | Adamtsl1 | 4.99E−41 |
| Endothelial | Stxbp6 | 2.89E−40 |
| Endothelial | Cdh5 | 1.44E−39 |
| Endothelial | Arap3 | 1.81E−39 |
| Endothelial | Gpm6a | 3.78E−39 |
| Endothelial | Arhgap31 | 1.37E−38 |
| Endothelial | Tcf4 | 3.24E−38 |
| Endothelial | Zbtb20 | 8.63E−38 |
| Endothelial | Sncaip | 1.16E−37 |
| Endothelial | Arhgap29 | 2.61E−37 |
| Endothelial | Prkch | 3.29E−37 |
| Endothelial | Grk5 | 5.49E−37 |
| Endothelial | Tmtc1 | 1.68E−36 |
| Endothelial | Prelp | 5.42E−36 |
| Endothelial | Tmsb4x | 6.44E−36 |
| Endothelial | Elmo1 | 2.28E−35 |
| Endothelial | Dysf | 3.84E−35 |
| Endothelial | Ptprb | 5.48E−35 |
| Endothelial | Ltbp4 | 8.26E−35 |
| Endothelial | Osmr | 3.53E−34 |
| Endothelial | Tgfbr2 | 3.92E−34 |
| Endothelial | Arl15 | 2.60E−33 |
| Endothelial | Ppfibp1 | 9.71E−33 |
| Endothelial | Ackr3 | 1.94E−32 |
| Endothelial | Syne1 | 2.33E−32 |
| Endothelial | Ifitm3 | 2.72E−32 |
| Endothelial | Trpc3 | 6.15E−32 |
| Endothelial | Slco2b1 | 7.30E−31 |
| Endothelial | Palm | 1.87E−30 |
| Endothelial | S1pr1 | 8.04E−30 |
| Endothelial | Lbp | 1.25E−29 |
| Endothelial | Eng | 7.13E−29 |
| Endothelial | Flt1 | 9.71E−27 |
| Endothelial | Gucy1b3 | 5.38E−26 |
| Endothelial | Adgrf5 | 7.37E−26 |
| Endothelial | Ramp2 | 2.93E−25 |
| Endothelial | 4930578C19Rik | 4.98E−25 |
| Endothelial | Nhsl2 | 5.93E−25 |
| Endothelial | Ecscr | 7.38E−25 |
| Endothelial | Kdr | 1.09E−24 |
| Endothelial | Thsd1 | 1.28E−24 |
| Endothelial | Tie1 | 2.13E−23 |
| Endothelial | Pkhd1l1 | 2.13E−23 |
| Endothelial | Ushbp1 | 6.36E−23 |
| Endothelial | Ets1 | 2.88E−22 |
| Endothelial | Stab1 | 6.10E−22 |
| Endothelial | Lmo2 | 3.35E−20 |
| Endothelial | Btnl9 | 4.22E−20 |
| Endothelial | Parvb | 7.19E−19 |
| Endothelial | Cd300lg | 1.36E−18 |
| Endothelial | Tbx1 | 1.01E−17 |
| Endothelial | Dtx1 | 2.42E−17 |
| Endothelial | Sh3gl3 | 4.92E−17 |
| Endothelial | Slc10a6 | 5.94E−16 |
| Endothelial | Sema3f | 6.89E−16 |
| Endothelial | 4833422C13Rik | 1.67E−15 |
| Endothelial | Apba2 | 6.23E−15 |
| Endothelial | Sept4 | 1.26E−14 |
| Endothelial | Iigp1 | 2.11E−14 |
| Endothelial | Ackr2 | 3.13E−14 |
| Endothelial | Cyp4b1 | 5.77E−14 |
| Endothelial | Scn1b | 6.04E−14 |
| Endothelial | 4930578G10Rik | 8.28E−14 |
| Endothelial | Gprc5b | 1.20E−13 |
| Endothelial | Erg | 2.87E−13 |
| Endothelial | D830026I12Rik | 3.53E−13 |
| Endothelial | Lrg1 | 1.16E−12 |
| Endothelial | Apold1 | 1.22E−12 |
| Endothelial | Ly6c1 | 3.59E−12 |
| Endothelial | Tal1 | 3.90E−12 |
| Endothelial | Islr2 | 6.84E−12 |
| Endothelial | Thbd | 1.49E−11 |
| Endothelial | Gpihbp1 | 4.40E−11 |
| Endothelial | Clec1a | 5.33E−11 |
| Endothelial | Ecm2 | 9.67E−11 |
| Endothelial | Arhgef15 | 4.77E−10 |
| Endothelial | Slfn3 | 5.88E−10 |
| Endothelial | Cd93 | 6.88E−10 |
| Endothelial | She | 9.89E−10 |
| Endothelial | Fmnl3 | 2.50E−08 |
| Endothelial | Plvap | 6.80E−07 |
| Enteroendocrine | Kcnb2 | 2.07E−89 |
| Enteroendocrine | Chgb | 5.11E−76 |
| Enteroendocrine | Cadps | 5.11E−76 |
| Enteroendocrine | Rimbp2 | 1.18E−73 |
| Enteroendocrine | Chga | 3.34E−72 |
| Enteroendocrine | Snap25 | 1.25E−69 |
| Enteroendocrine | Scn3a | 1.31E−66 |
| Enteroendocrine | Rgs7 | 1.71E−61 |
| Enteroendocrine | Slc38a11 | 3.57E−60 |
| Enteroendocrine | Adcy2 | 4.94E−57 |
| Enteroendocrine | Cacna2d1 | 5.09E−55 |
| Enteroendocrine | Ctnna2 | 1.10E−54 |
| Enteroendocrine | Cacna1a | 2.67E−54 |
| Enteroendocrine | Runx1t1 | 5.85E−54 |
| Enteroendocrine | Ddc | 5.85E−54 |
| Enteroendocrine | 1700042O10Rik | 3.50E−53 |
| Enteroendocrine | Cerkl | 4.85E−51 |
| Enteroendocrine | Nrxn1 | 4.69E−50 |
| Enteroendocrine | Rfx6 | 8.58E−48 |
| Enteroendocrine | Tph1 | 1.91E−44 |
| Enteroendocrine | Ptprn2 | 4.21E−42 |
| Enteroendocrine | Pyy | 8.13E−42 |
| Enteroendocrine | Cpe | 1.64E−41 |
| Enteroendocrine | Lmx1a | 7.23E−41 |
| Enteroendocrine | Gm609 | 3.05E−40 |
| Enteroendocrine | Fam19a1 | 2.80E−38 |
| Enteroendocrine | Vwa5b2 | 5.28E−38 |
| Enteroendocrine | Map2 | 5.71E−38 |
| Enteroendocrine | Rab3c | 9.55E−37 |
| Enteroendocrine | Pam | 2.40E−36 |
| Enteroendocrine | St18 | 9.47E−36 |
| Enteroendocrine | Rfx3 | 7.40E−34 |
| Enteroendocrine | Slc18a1 | 8.62E−34 |
| Enteroendocrine | Pcsk1n | 2.76E−33 |
| Enteroendocrine | Fam105a | 4.66E−33 |
| Enteroendocrine | Gcg | 6.45E−32 |
| Enteroendocrine | Map1b | 5.03E−31 |
| Enteroendocrine | Pclo | 4.64E−30 |
| Enteroendocrine | Sct | 5.19E−30 |
| Enteroendocrine | Enox1 | 9.64E−30 |
| Enteroendocrine | Rora | 1.23E−29 |
| Enteroendocrine | Olfr78 | 2.73E−29 |
| Enteroendocrine | Stxbp5l | 3.28E−28 |
| Enteroendocrine | Pde4d | 5.07E−28 |
| Enteroendocrine | Nkx2-2 | 1.87E−27 |
| Enteroendocrine | Slc8a1 | 1.09E−26 |
| Enteroendocrine | Insl5 | 5.97E−26 |
| Enteroendocrine | Jazf1 | 1.13E−24 |
| Enteroendocrine | Hmgn3 | 1.21E−23 |
| Enteroendocrine | Etv1 | 7.28E−23 |
| Enteroendocrine | Myt1 | 2.38E−22 |
| Enteroendocrine | Otud7a | 4.44E−22 |
| Enteroendocrine | Kcnh7 | 6.08E−22 |
| Enteroendocrine | Scg5 | 4.06E−21 |
| Enteroendocrine | 1810006J02Rik | 5.70E−21 |
| Enteroendocrine | Unc79 | 9.51E−21 |
| Enteroendocrine | Pex5l | 1.70E−20 |
| Enteroendocrine | Ctnnd2 | 1.85E−20 |
| Enteroendocrine | Fry | 1.44E−19 |
| Enteroendocrine | Isl1 | 1.99E−19 |
| Enteroendocrine | Piezo2 | 2.15E−19 |
| Enteroendocrine | Asic2 | 4.14E−19 |
| Enteroendocrine | Ptprn | 4.60E−19 |
| Enteroendocrine | Celf3 | 8.34E−19 |
| Enteroendocrine | Gfra3 | 1.34E−18 |
| Enteroendocrine | Kcnmb2 | 8.80E−18 |
| Enteroendocrine | Kcnh8 | 2.73E−17 |
| Enteroendocrine | Ghr | 3.34E−17 |
| Enteroendocrine | Man1c1 | 3.82E−17 |
| Enteroendocrine | Insm1 | 3.82E−17 |
| Enteroendocrine | Zbtb20 | 5.18E−17 |
| Enteroendocrine | Glis3 | 5.92E−17 |
| Enteroendocrine | Cyp4x1 | 6.48E−17 |
| Enteroendocrine | Ptprt | 7.80E−17 |
| Enteroendocrine | Negr1 | 1.90E−16 |
| Enteroendocrine | Rims2 | 2.27E−16 |
| Enteroendocrine | Pappa2 | 2.32E−16 |
| Enteroendocrine | Dach1 | 2.54E−16 |
| Enteroendocrine | Pax6 | 3.11E−16 |
| Enteroendocrine | Syn2 | 5.82E−16 |
| Enteroendocrine | Stk32a | 5.83E−16 |
| Enteroendocrine | Nbea | 7.39E−16 |
| Enteroendocrine | Nrg1 | 1.29E−15 |
| Enteroendocrine | Wif1 | 1.59E−15 |
| Enteroendocrine | Cacna1c | 1.75E−15 |
| Enteroendocrine | Pde11a | 4.19E−15 |
| Enteroendocrine | Gnao1 | 6.72E−15 |
| Enteroendocrine | Astn2 | 9.59E−15 |
| Enteroendocrine | Phldb2 | 2.12E−14 |
| Enteroendocrine | Scg3 | 3.82E−14 |
| Enteroendocrine | Rasal2 | 4.28E−14 |
| Enteroendocrine | Rap1gap2 | 4.39E−14 |
| Enteroendocrine | Nxph1 | 5.71E−14 |
| Enteroendocrine | Itpr1 | 6.50E−14 |
| Enteroendocrine | Resp18 | 7.37E−14 |
| Enteroendocrine | Robo1 | 2.25E−13 |
| Enteroendocrine | Cacnb2 | 3.12E−13 |
| Enteroendocrine | Lin7a | 3.63E−13 |
| Enteroendocrine | Rundc3a | 5.66E−13 |
| Enteroendocrine | Lcorl | 5.92E−13 |
| Enteroendocrine | Peg3 | 1.04E−12 |
| Enteroendocrine | Pax6os1 | 1.40E−12 |
| Enteroendocrine | Gpr119 | 1.50E−12 |
| Enteroendocrine | Cck | 2.26E−11 |
| Enteroendocrine | Lrrn3 | 2.26E−11 |
| Enteroendocrine | Slc29a4 | 4.21E−11 |
| Enteroendocrine | Nfasc | 6.62E−11 |
| Enteroendocrine | March4 | 2.69E−10 |
| Enteroendocrine | Amigo2 | 3.24E−10 |
| Enteroendocrine | Mreg | 3.94E−10 |
| Enteroendocrine | Unc13a | 1.20E−09 |
| Enteroendocrine | Slc6a19 | 1.21E−09 |
| Enteroendocrine | Avpr1b | 1.21E−09 |
| Enteroendocrine | Galr1 | 2.47E−09 |
| Enteroendocrine | Kcnk3 | 4.87E−09 |
| Enteroendocrine | Iapp | 8.92E−09 |
| Enteroendocrine | Baiap3 | 1.76E−08 |
| Enteroendocrine | Serpinf2 | 6.40E−08 |
| Enteroendocrine | Scgn | 6.40E−08 |
| Enteroendocrine | Cryba2 | 6.40E−08 |
| Enteroendocrine | Cxxc4 | 8.62E−08 |
| Enteroendocrine | Gsdma | 9.32E−08 |
| Enteroendocrine | Ace2 | 9.87E−08 |
| Enteroendocrine | Igsf21 | 1.08E−07 |
| Enteroendocrine | Rcan2 | 1.14E−07 |
| Enteroendocrine | Tm4sf4 | 1.38E−07 |
| Enteroendocrine | Gipr | 1.90E−07 |
| Enteroendocrine | Fam20c | 3.20E−07 |
| Enteroendocrine | Gm15716 | 4.45E−07 |
| Enteroendocrine | Miat | 4.63E−07 |
| Enteroendocrine | Slc26a4 | 5.53E−07 |
| Enteroendocrine | Gdap1l1 | 6.82E−07 |
| Enteroendocrine | Gm17276 | 2.27E−06 |
| Enteroendocrine | Maats1 | 3.62E−06 |
| Enteroendocrine | 4931429I11Rik | 3.79E−06 |
| Enteroendocrine | Slc35d3 | 8.56E−06 |
| Enteroendocrine | Unc5a | 1.09E−05 |
| Enteroendocrine | Dcx | 1.42E−05 |
| Enteroendocrine | Gck | 2.04E−05 |
| Enteroendocrine | Syndig1l | 2.44E−05 |
| Enteroendocrine | Sez6l2 | 6.45E−05 |
| Enteroendocrine | AW551984 | 6.92E−05 |
| Enteroendocrine | Dnaic1 | 1.04E−04 |
| Enteroendocrine | Sst | 1.16E−04 |
| Enteroendocrine | Rimkla | 1.58E−04 |
| Enteroendocrine | Elavl2 | 3.38E−04 |
| Enteroendocrine | Gm27162 | 7.60E−04 |
| Epithelial_Progenitors | Gmds | 3.61E−141 |
| Epithelial_Progenitors | Ntan1 | 2.40E−78 |
| Epithelial_Progenitors | 5330417C22Rik | 8.03E−78 |
| Epithelial_Progenitors | Gfpt1 | 2.63E−75 |
| Epithelial_Progenitors | Fut8 | 2.30E−73 |
| Epithelial_Progenitors | Tox | 1.55E−70 |
| Epithelial_Progenitors | Pdxdc1 | 1.03E−67 |
| Epithelial_Progenitors | Golph3l | 4.55E−65 |
| Epithelial_Progenitors | Airn | 6.54E−65 |
| Epithelial_Progenitors | 9030622O22Rik | 4.00E−64 |
| Epithelial_Progenitors | Arhgef38 | 2.79E−63 |
| Epithelial_Progenitors | Slc12a8 | 6.01E−63 |
| Epithelial_Progenitors | Oit1 | 3.89E−58 |
| Epithelial_Progenitors | Fam13a | 7.30E−58 |
| Epithelial_Progenitors | Mecom | 1.45E−53 |
| Epithelial_Progenitors | Slc35a3 | 2.51E−51 |
| Epithelial_Progenitors | Galnt7 | 2.72E−49 |
| Epithelial_Progenitors | Gne | 2.72E−47 |
| Epithelial_Progenitors | Creb3l1 | 4.10E−41 |
| Epithelial_Progenitors | Sorbs2 | 4.65E−40 |
| Epithelial_Progenitors | Mcc | 4.65E−40 |
| Epithelial_Progenitors | Slc12a2 | 9.96E−39 |
| Epithelial_Progenitors | Klf5 | 7.87E−37 |
| Epithelial_Progenitors | Naaladl2 | 1.92E−36 |
| Epithelial_Progenitors | Gm26848 | 2.92E−36 |
| Epithelial_Progenitors | Greb1l | 6.02E−35 |
| Epithelial_Progenitors | Sidt1 | 9.63E−35 |
| Epithelial_Progenitors | Vps13b | 9.76E−35 |
| Epithelial_Progenitors | Rgs17 | 1.24E−34 |
| Epithelial_Progenitors | Spdef | 6.75E−34 |
| Epithelial_Progenitors | Myo3a | 1.54E−33 |
| Epithelial_Progenitors | Prkca | 3.85E−33 |
| Epithelial_Progenitors | Nupr1 | 1.74E−32 |
| Epithelial_Progenitors | Ptprk | 2.72E−32 |
| Epithelial_Progenitors | Car8 | 5.18E−32 |
| Epithelial_Progenitors | Gcc2 | 6.90E−32 |
| Epithelial_Progenitors | Ehf | 8.03E−32 |
| Epithelial_Progenitors | Mia3 | 1.66E−31 |
| Epithelial_Progenitors | Camk1d | 3.50E−30 |
| Epithelial_Progenitors | Tnfaip8 | 1.14E−29 |
| Epithelial_Progenitors | Arhgef28 | 2.30E−29 |
| Epithelial_Progenitors | Agr2 | 4.35E−29 |
| Epithelial_Progenitors | Klf12 | 5.57E−29 |
| Epithelial_Progenitors | Rapgef5 | 6.74E−29 |
| Epithelial_Progenitors | Nfib | 1.55E−27 |
| Epithelial_Progenitors | Nfia | 1.60E−27 |
| Epithelial_Progenitors | Kcnma1 | 3.78E−27 |
| Epithelial_Progenitors | Etv5 | 3.78E−27 |
| Epithelial_Progenitors | Col8a1 | 3.78E−27 |
| Epithelial_Progenitors | Galnt10 | 3.78E−27 |
| Epithelial_Progenitors | Gm609 | 5.02E−27 |
| Epithelial_Progenitors | Kank1 | 6.40E−27 |
| Epithelial_Progenitors | St3gal6 | 9.50E−27 |
| Epithelial_Progenitors | Ptprt | 1.22E−26 |
| Epithelial_Progenitors | Ephb2 | 2.09E−26 |
| Epithelial_Progenitors | Satb2 | 2.33E−26 |
| Epithelial_Progenitors | Pawr | 4.28E−26 |
| Epithelial_Progenitors | Chrm3 | 6.85E−26 |
| Epithelial_Progenitors | Pla2g4a | 1.97E−25 |
| Epithelial_Progenitors | Tbc1d4 | 3.16E−25 |
| Epithelial_Progenitors | Slc50a1 | 5.21E−25 |
| Epithelial_Progenitors | Fut2 | 5.69E−25 |
| Epithelial_Progenitors | St6galnac6 | 1.15E−24 |
| Epithelial_Progenitors | Atp8b1 | 7.23E−24 |
| Epithelial_Progenitors | Ern2 | 1.35E−23 |
| Epithelial_Progenitors | Ica1 | 1.67E−23 |
| Epithelial_Progenitors | 0610040J01Rik | 3.63E−23 |
| Epithelial_Progenitors | Neat1 | 7.17E−23 |
| Epithelial_Progenitors | Tc2n | 9.21E−23 |
| Epithelial_Progenitors | Pdia5 | 1.72E−22 |
| Epithelial_Progenitors | Arhgap24 | 2.08E−22 |
| Epithelial_Progenitors | Ptprn2 | 7.60E−22 |
| Epithelial_Progenitors | Mettl23 | 1.49E−21 |
| Epithelial_Progenitors | Cadps2 | 1.99E−21 |
| Epithelial_Progenitors | Ralgapa2 | 3.07E−21 |
| Epithelial_Progenitors | Slc1a5 | 4.45E−21 |
| Epithelial_Progenitors | Tmem181a | 4.87E−21 |
| Epithelial_Progenitors | Tpd52 | 5.27E−21 |
| Epithelial_Progenitors | Rsrp1 | 5.31E−21 |
| Epithelial_Progenitors | Cdk6 | 8.30E−21 |
| Epithelial_Progenitors | Galnt3 | 1.51E−20 |
| Epithelial_Progenitors | Slc17a9 | 1.68E−20 |
| Epithelial_Progenitors | Thrb | 3.03E−20 |
| Epithelial_Progenitors | Plcb4 | 4.04E−20 |
| Epithelial_Progenitors | St3gal3 | 4.43E−20 |
| Epithelial_Progenitors | Sybu | 4.75E−20 |
| Epithelial_Progenitors | Rbm39 | 7.85E−20 |
| Epithelial_Progenitors | Mgat5 | 7.98E−20 |
| Epithelial_Progenitors | C1galt1 | 8.14E−20 |
| Epithelial_Progenitors | Pmm2 | 1.35E−19 |
| Epithelial_Progenitors | Mctp1 | 1.36E−19 |
| Epithelial_Progenitors | Vgll4 | 2.71E−19 |
| Epithelial_Progenitors | Ppp2r3a | 4.19E−19 |
| Epithelial_Progenitors | Utrn | 5.26E−19 |
| Epithelial_Progenitors | Cftr | 5.35E−19 |
| Epithelial_Progenitors | Arid5b | 6.17E−19 |
| Epithelial_Progenitors | Chchd3 | 9.43E−19 |
| Epithelial_Progenitors | Acer3 | 1.10E−18 |
| Epithelial_Progenitors | Uhrf2 | 1.24E−18 |
| Epithelial_Progenitors | Sgsm3 | 1.25E−18 |
| Epithelial_Progenitors | Slc1a4 | 3.62E−18 |
| Epithelial_Progenitors | Hes6 | 7.77E−18 |
| Epithelial_Progenitors | Nxpe2 | 6.41E−17 |
| Epithelial_Progenitors | Gm13832 | 7.57E−17 |
| Epithelial_Progenitors | Lpcat2 | 9.70E−16 |
| Epithelial_Progenitors | Bsn | 1.21E−15 |
| Epithelial_Progenitors | Dync1i1 | 3.33E−15 |
| Epithelial_Progenitors | Kit | 1.65E−14 |
| Epithelial_Progenitors | Fam189a1 | 1.56E−13 |
| Epithelial_Progenitors | AI838599 | 9.21E−13 |
| Epithelial_Progenitors | Samd5 | 7.54E−12 |
| Epithelial_Progenitors | Cbfa2t3 | 1.01E−11 |
| Epithelial_Progenitors | Clca3a2 | 2.03E−11 |
| Epithelial_Progenitors | Hiat1 | 4.29E−11 |
| Epithelial_Progenitors | Gm26908 | 6.63E−11 |
| Epithelial_Progenitors | Pck1 | 1.07E−10 |
| Epithelial_Progenitors | Rnf32 | 1.12E−10 |
| Epithelial_Progenitors | Mettl1 | 1.62E−10 |
| Epithelial_Progenitors | Tpcn2 | 2.98E−10 |
| Epithelial_Progenitors | C2cd4b | 5.47E−10 |
| Epithelial_Progenitors | A4gnt | 2.98E−09 |
| Epithelial_Progenitors | Meg3 | 3.97E−09 |
| Epithelial_Progenitors | Gm13247 | 3.98E−09 |
| Epithelial_Progenitors | Uck2 | 4.08E−09 |
| Epithelial_Progenitors | Clca3b | 5.14E−09 |
| Epithelial_Progenitors | Pgm3 | 1.69E−08 |
| Epithelial_Progenitors | Rhbdl3 | 1.71E−08 |
| Epithelial_Progenitors | Gm12860 | 1.75E−08 |
| Epithelial_Progenitors | Hopx | 1.82E−08 |
| Epithelial_Progenitors | Rab15 | 3.44E−08 |
| Epithelial_Progenitors | Fam98a | 3.76E−08 |
| Epithelial_Progenitors | Slc16a7 | 4.41E−08 |
| Epithelial_Progenitors | Mcoln2 | 1.25E−07 |
| Epithelial_Progenitors | 4933406C10Rik | 4.06E−07 |
| Epithelial_Progenitors | Slc39a8 | 5.57E−07 |
| Epithelial_Progenitors | Pex5l | 5.86E−07 |
| Epithelial_Progenitors | Kif15 | 9.97E−07 |
| Epithelial_Progenitors | Eepd1 | 1.03E−06 |
| Epithelial_Progenitors | Ccdc125 | 1.17E−06 |
| Epithelial_Progenitors | Pacsin1 | 1.51E−06 |
| Epithelial_Progenitors | Gm14342 | 1.64E−06 |
| Epithelial_Progenitors | Kcnh3 | 2.00E−06 |
| Epithelial_Progenitors | Inpp1 | 2.40E−06 |
| Epithelial_Progenitors | Creb3l4 | 2.89E−06 |
| Epithelial_Progenitors | Lgals12 | 1.09E−05 |
| Epithelial_Progenitors | 9430060I03Rik | 1.34E−05 |
| Epithelial_Progenitors | Mmp28 | 1.68E−05 |
| Epithelial_Progenitors | Gm43191 | 2.45E−05 |
| Epithelial_Progenitors | Kif11 | 2.65E−05 |
| Epithelial_Progenitors | Neil3 | 3.63E−05 |
| Epithelial_Progenitors | Wdr76 | 3.96E−05 |
| Epithelial_Progenitors | Pnmal2 | 4.18E−05 |
| Epithelial_Progenitors | Palmd | 4.72E−05 |
| Epithelial_Progenitors | Acsl1 | 6.66E−05 |
| Epithelial_Progenitors | Smoc2 | 6.71E−05 |
| Epithelial_Progenitors | Abo | 7.52E−05 |
| Epithelial_Progenitors | Fut9 | 8.90E−05 |
| Epithelial_Progenitors | Fbxo21 | 1.00E−04 |
| Epithelial_Progenitors | Triqk | 1.13E−04 |
| Epithelial_Progenitors | D930020B18Rik | 1.71E−04 |
| Epithelial_Progenitors | Ttc39aos1 | 1.91E−04 |
| Epithelial_Progenitors | Zwilch | 3.75E−04 |
| Epithelial_Progenitors | Plk2 | 4.10E−04 |
| Epithelial_Progenitors | Gm15848 | 4.48E−04 |
| Epithelial_Progenitors | Mastl | 4.81E−04 |
| Epithelial_Progenitors | Pik3c2g | 5.12E−04 |
| Epithelial_Progenitors | N6amt1 | 5.95E−04 |
| Epithelial_Progenitors | Spc24 | 7.12E−04 |
| Epithelial_Progenitors | Wdhd1 | 7.36E−04 |
| Epithelial_Progenitors | Zfp612 | 1.29E−03 |
| Epithelial_Progenitors | Agtr1b | 1.74E−03 |
| Epithelial_Progenitors | Isyna1 | 2.52E−03 |
| Epithelial_Progenitors | 1700013F07Rik | 4.27E−03 |
| Epithelial_Progenitors.1 | Rpl41 | 1.86E−115 |
| Epithelial_Progenitors.1 | Rpl23 | 5.03E−93 |
| Epithelial_Progenitors.1 | Rplp1 | 1.96E−89 |
| Epithelial_Progenitors.1 | Gm10073 | 4.09E−87 |
| Epithelial_Progenitors.1 | Gm8730 | 4.20E−83 |
| Epithelial_Progenitors.1 | Rps3 | 4.65E−82 |
| Epithelial_Progenitors.1 | mt-Cytb | 1.08E−79 |
| Epithelial_Progenitors.1 | Rps19 | 2.55E−79 |
| Epithelial_Progenitors.1 | Rps9 | 1.60E−77 |
| Epithelial_Progenitors.1 | Rps24 | 2.92E−77 |
| Epithelial_Progenitors.1 | Rpl37 | 3.68E−77 |
| Epithelial_Progenitors.1 | Rpl19 | 3.68E−77 |
| Epithelial_Progenitors.1 | Rps23 | 3.73E−77 |
| Epithelial_Progenitors.1 | Rpl9-ps6 | 4.29E−77 |
| Epithelial_Progenitors.1 | Rps14 | 3.67E−76 |
| Epithelial_Progenitors.1 | Rps18 | 3.67E−76 |
| Epithelial_Progenitors.1 | Rps8 | 3.67E−76 |
| Epithelial_Progenitors.1 | Rpl32 | 9.28E−76 |
| Epithelial_Progenitors.1 | Rps15 | 9.76E−76 |
| Epithelial_Progenitors.1 | Pigr | 4.26E−75 |
| Epithelial_Progenitors.1 | Cox8a | 5.16E−74 |
| Epithelial_Progenitors.1 | Eef1a1 | 4.33E−73 |
| Epithelial_Progenitors.1 | Rpl26 | 2.45E−72 |
| Epithelial_Progenitors.1 | Rpl13a | 3.64E−72 |
| Epithelial_Progenitors.1 | mt-Nd4 | 6.24E−72 |
| Epithelial_Progenitors.1 | Wdr89 | 4.96E−71 |
| Epithelial_Progenitors.1 | Rps2 | 5.61E−71 |
| Epithelial_Progenitors.1 | Rpl21 | 1.29E−70 |
| Epithelial_Progenitors.1 | mt-Co2 | 1.29E−70 |
| Epithelial_Progenitors.1 | mt-Atp6 | 1.52E−70 |
| Epithelial_Progenitors.1 | Rpl8 | 8.16E−70 |
| Epithelial_Progenitors.1 | Rpl11 | 2.05E−69 |
| Epithelial_Progenitors.1 | Rpl18a | 2.85E−69 |
| Epithelial_Progenitors.1 | Rpl35a | 1.65E−68 |
| Epithelial_Progenitors.1 | mt-Nd1 | 2.55E−68 |
| Epithelial_Progenitors.1 | Rps16 | 4.06E−68 |
| Epithelial_Progenitors.1 | Rps4x | 5.10E−67 |
| Epithelial_Progenitors.1 | mt-Co3 | 2.37E−66 |
| Epithelial_Progenitors.1 | Rpl37a | 3.78E−66 |
| Epithelial_Progenitors.1 | Rps27a | 5.37E−66 |
| Epithelial_Progenitors.1 | Rps20 | 5.73E−65 |
| Epithelial_Progenitors.1 | Rpip0 | 6.50E−65 |
| Epithelial_Progenitors.1 | Rpsa | 8.50E−65 |
| Epithelial_Progenitors.1 | Rps5 | 1.87E−64 |
| Epithelial_Progenitors.1 | Gm10036 | 4.50E−64 |
| Epithelial_Progenitors.1 | Rpl10a | 1.18E−63 |
| Epithelial_Progenitors.1 | Rpl7 | 2.92E−63 |
| Epithelial_Progenitors.1 | Uqcrh | 5.51E−63 |
| Epithelial_Progenitors.1 | Rpl27a | 2.89E−62 |
| Epithelial_Progenitors.1 | Rpl18 | 5.88E−62 |
| Epithelial_Progenitors.1 | Mt1 | 3.39E−61 |
| Epithelial_Progenitors.1 | Rpl28 | 4.94E−61 |
| Epithelial_Progenitors.1 | Rps6 | 7.59E−61 |
| Epithelial_Progenitors.1 | Rps11 | 1.24E−59 |
| Epithelial_Progenitors.1 | Scd2 | 7.56E−59 |
| Epithelial_Progenitors.1 | Rpl14 | 1.02E−58 |
| Epithelial_Progenitors.1 | Rpl4 | 1.08E−57 |
| Epithelial_Progenitors.1 | Rps29 | 3.22E−57 |
| Epithelial_Progenitors.1 | Gm9843 | 3.33E−57 |
| Epithelial_Progenitors.1 | Rps26 | 9.08E−57 |
| Epithelial_Progenitors.1 | Gpx2 | 1.64E−54 |
| Epithelial_Progenitors.1 | Rpl24 | 8.24E−54 |
| Epithelial_Progenitors.1 | Rpl13-ps3 | 9.49E−54 |
| Epithelial_Progenitors.1 | Rps17 | 1.25E−53 |
| Epithelial_Progenitors.1 | Cox6c | 2.59E−53 |
| Epithelial_Progenitors.1 | Uqcr10 | 8.97E−53 |
| Epithelial_Progenitors.1 | Tecpr2 | 4.91E−52 |
| Epithelial_Progenitors.1 | mt-Co1 | 3.30E−51 |
| Epithelial_Progenitors.1 | Rps26-ps1 | 6.74E−51 |
| Epithelial_Progenitors.1 | Rpl36al | 3.23E−50 |
| Epithelial_Progenitors.1 | Rpl29 | 6.33E−50 |
| Epithelial_Progenitors.1 | Rps10 | 1.18E−49 |
| Epithelial_Progenitors.1 | Cox6a1 | 6.43E−49 |
| Epithelial_Progenitors.1 | Rps27rt | 9.46E−49 |
| Epithelial_Progenitors.1 | Rpl39 | 1.81E−48 |
| Epithelial_Progenitors.1 | Rpl6l | 3.04E−48 |
| Epithelial_Progenitors.1 | Rps18-ps3 | 6.15E−48 |
| Epithelial_Progenitors.1 | Rps3a1 | 3.15E−47 |
| Epithelial_Progenitors.1 | Tpt1 | 3.84E−45 |
| Epithelial_Progenitors.1 | Rps25 | 1.97E−44 |
| Epithelial_Progenitors.1 | Ftl1 | 1.10E−43 |
| Epithelial_Progenitors.1 | Ddah1 | 2.39E−43 |
| Epithelial_Progenitors.1 | Rps21 | 5.06E−43 |
| Epithelial_Progenitors.1 | Rpl5 | 5.50E−42 |
| Epithelial_Progenitors.1 | Rpl27-ps3 | 6.68E−42 |
| Epithelial_Progenitors.1 | Ptma | 7.74E−42 |
| Epithelial_Progenitors.1 | Ctla4 | 4.76E−41 |
| Epithelial_Progenitors.1 | Nrg1 | 3.98E−40 |
| Epithelial_Progenitors.1 | Sycn | 1.18E−39 |
| Epithelial_Progenitors.1 | Gm10263 | 2.01E−39 |
| Epithelial_Progenitors.1 | mt-Nd2 | 3.28E−39 |
| Epithelial_Progenitors.1 | Atp5e | 4.45E−39 |
| Epithelial_Progenitors.1 | Rpl38 | 6.37E−39 |
| Epithelial_Progenitors.1 | Rps28 | 1.02E−38 |
| Epithelial_Progenitors.1 | Gnb2l1 | 1.49E−37 |
| Epithelial_Progenitors.1 | Sumf1 | 7.13E−37 |
| Epithelial_Progenitors.1 | Rpl17 | 8.97E−37 |
| Epithelial_Progenitors.1 | Rpl34 | 9.85E−37 |
| Epithelial_Progenitors.1 | Eef1g | 5.38E−36 |
| Epithelial_Progenitors.1 | Eef1b2 | 1.73E−35 |
| Epithelial_Progenitors.1 | Gm9493 | 3.01E−35 |
| Epithelial_Progenitors.1 | Rps27l | 4.31E−35 |
| Epithelial_Progenitors.1 | Uqcr11 | 1.90E−34 |
| Epithelial_Progenitors.1 | Slc5a8 | 2.44E−33 |
| Epithelial_Progenitors.1 | Atp5g1 | 2.51E−33 |
| Epithelial_Progenitors.1 | Atp5g2 | 5.27E−30 |
| Epithelial_Progenitors.1 | Wfdc2 | 4.18E−29 |
| Epithelial_Progenitors.1 | Mt2 | 8.75E−29 |
| Epithelial_Progenitors.1 | Hsp90ab1 | 9.95E−28 |
| Epithelial_Progenitors.1 | Rps12-ps3 | 4.23E−22 |
| Epithelial_Progenitors.1 | Anapc13 | 4.57E−19 |
| Epithelial_Progenitors.1 | Gm8186 | 6.05E−19 |
| Epithelial_Progenitors.1 | Hoxb13 | 2.85E−16 |
| Epithelial_Progenitors.1 | Lsm4 | 6.99E−15 |
| Epithelial_Progenitors.1 | 2410015M20Rik | 1.92E−14 |
| Epithelial_Progenitors.1 | Banf1 | 2.56E−14 |
| Epithelial_Progenitors.1 | Mgst1 | 3.75E−14 |
| Epithelial_Progenitors.1 | Spink4 | 6.36E−14 |
| Epithelial_Progenitors.1 | Mei4 | 1.12E−13 |
| Epithelial_Progenitors.1 | Gm15013 | 1.27E−11 |
| Epithelial_Progenitors.1 | Romo1 | 4.04E−11 |
| Epithelial_Progenitors.1 | Cyp2c68 | 5.31E−11 |
| Epithelial_Progenitors.1 | Gm11273 | 5.35E−11 |
| Epithelial_Progenitors.1 | Cfap46 | 5.65E−11 |
| Epithelial_Progenitors.1 | Rab25 | 8.98E−11 |
| Epithelial_Progenitors.1 | Nhp2 | 3.84E−10 |
| Epithelial_Progenitors.1 | Lsm5 | 4.08E−10 |
| Epithelial_Progenitors.1 | Impdh2 | 4.37E−10 |
| Epithelial_Progenitors.1 | Ndufa8 | 1.06E−09 |
| Epithelial_Progenitors.1 | Smim11 | 5.93E−09 |
| Epithelial_Progenitors.1 | Uba52 | 6.93E−09 |
| Epithelial_Progenitors.1 | Fut4 | 1.05E−08 |
| Epithelial_Progenitors.1 | Birc5 | 2.33E−08 |
| Epithelial_Progenitors.1 | Plbd1 | 1.16E−07 |
| Epithelial_Progenitors.1 | Unc119 | 1.31E−07 |
| Epithelial_Progenitors.1 | Ube2c | 3.24E−07 |
| Epithelial_Progenitors.1 | Slc35b2 | 3.42E−07 |
| Epithelial_Progenitors.1 | Hmgn2 | 5.59E−07 |
| Epithelial_Progenitors.1 | Rpl27 | 1.03E−06 |
| Epithelial_Progenitors.1 | Cdca3 | 1.04E−06 |
| Epithelial_Progenitors.1 | Cebpzos | 2.22E−06 |
| Epithelial_Progenitors.1 | Knstrn | 4.65E−06 |
| Epithelial_Progenitors.1 | Ran | 7.01E−06 |
| Epithelial_Progenitors.1 | Pdzk1ip1 | 8.00E−06 |
| Epithelial_Progenitors.1 | Tspan33 | 1.12E−05 |
| Epithelial_Progenitors.1 | Tmem192 | 2.90E−05 |
| Epithelial_Progenitors.1 | Yif1a | 7.48E−05 |
| Epithelial_Progenitors.1 | Snrnp25 | 2.63E−04 |
| Epithelial_Progenitors.1 | Gm10053 | 5.20E−04 |
| Epithelial_Progenitors.2 | Hsd3b3 | 1.85E−121 |
| Epithelial_Progenitors.2 | Gsdmc2 | 2.52E−86 |
| Epithelial_Progenitors.2 | Dmbt1 | 9.47E−73 |
| Epithelial_Progenitors.2 | Gsdmc4 | 1.95E−71 |
| Epithelial_Progenitors.2 | Hao2 | 3.17E−70 |
| Epithelial_Progenitors.2 | Cftr | 2.52E−67 |
| Epithelial_Progenitors.2 | Hnf4g | 2.01E−65 |
| Epithelial_Progenitors.2 | Gm26917 | 5.05E−64 |
| Epithelial_Progenitors.2 | Sult1a1 | 1.23E−59 |
| Epithelial_Progenitors.2 | Hmgcs2 | 3.70E−58 |
| Epithelial_Progenitors.2 | Chd9 | 4.13E−58 |
| Epithelial_Progenitors.2 | Satb2 | 2.08E−55 |
| Epithelial_Progenitors.2 | Cyp2c55 | 4.48E−52 |
| Epithelial_Progenitors.2 | Apol10a | 1.82E−48 |
| Epithelial_Progenitors.2 | Slc8a1 | 8.43E−46 |
| Epithelial_Progenitors.2 | Gsdmc3 | 1.40E−44 |
| Epithelial_Progenitors.2 | Slc5a8 | 7.50E−43 |
| Epithelial_Progenitors.2 | Fkbp5 | 9.49E−43 |
| Epithelial_Progenitors.2 | Dgkh | 2.20E−42 |
| Epithelial_Progenitors.2 | Mecom | 4.68E−42 |
| Epithelial_Progenitors.2 | Adk | 1.16E−40 |
| Epithelial_Progenitors.2 | Aqp4 | 1.87E−40 |
| Epithelial_Progenitors.2 | Pcsk5 | 6.16E−40 |
| Epithelial_Progenitors.2 | Vwa8 | 2.91E−38 |
| Epithelial_Progenitors.2 | Pparg | 2.91E−38 |
| Epithelial_Progenitors.2 | Immp2l | 2.07E−32 |
| Epithelial_Progenitors.2 | Krt19 | 3.06E−32 |
| Epithelial_Progenitors.2 | Slc26a3 | 3.96E−32 |
| Epithelial_Progenitors.2 | Ptprd | 1.32E−31 |
| Epithelial_Progenitors.2 | Ahcyl2 | 1.94E−30 |
| Epithelial_Progenitors.2 | Pof1b | 4.25E−29 |
| Epithelial_Progenitors.2 | Trim2 | 5.45E−29 |
| Epithelial_Progenitors.2 | B4galnt2 | 6.81E−29 |
| Epithelial_Progenitors.2 | Coro2a | 8.17E−29 |
| Epithelial_Progenitors.2 | Paqr5 | 8.18E−28 |
| Epithelial_Progenitors.2 | Papss2 | 9.48E−27 |
| Epithelial_Progenitors.2 | Mgat4c | 2.45E−25 |
| Epithelial_Progenitors.2 | Ptprk | 1.14E−24 |
| Epithelial_Progenitors.2 | Nr5a2 | 3.75E−24 |
| Epithelial_Progenitors.2 | Magi1 | 8.23E−24 |
| Epithelial_Progenitors.2 | Pdss2 | 8.43E−24 |
| Epithelial_Progenitors.2 | Nr1h4 | 1.42E−23 |
| Epithelial_Progenitors.2 | Hsd3b2 | 4.73E−23 |
| Epithelial_Progenitors.2 | Pde3a | 1.85E−22 |
| Epithelial_Progenitors.2 | Plekha5 | 2.43E−22 |
| Epithelial_Progenitors.2 | Mboat1 | 2.50E−22 |
| Epithelial_Progenitors.2 | Fgd4 | 3.52E−22 |
| Epithelial_Progenitors.2 | Gipc2 | 3.73E−22 |
| Epithelial_Progenitors.2 | Maoa | 1.46E−21 |
| Epithelial_Progenitors.2 | Acnat1 | 1.64E−21 |
| Epithelial_Progenitors.2 | Flnb | 4.07E−21 |
| Epithelial_Progenitors.2 | Pla2g3 | 5.57E−21 |
| Epithelial_Progenitors.2 | Gm42418 | 5.92E−21 |
| Epithelial_Progenitors.2 | Car1 | 8.45E−21 |
| Epithelial_Progenitors.2 | Ugdh | 8.86E−21 |
| Epithelial_Progenitors.2 | Clint1 | 1.21E−20 |
| Epithelial_Progenitors.2 | Plekha6 | 1.43E−20 |
| Epithelial_Progenitors.2 | Myo1d | 1.50E−20 |
| Epithelial_Progenitors.2 | Htr4 | 1.61E−20 |
| Epithelial_Progenitors.2 | Nfe2l2 | 5.34E−20 |
| Epithelial_Progenitors.2 | Xist | 1.48E−19 |
| Epithelial_Progenitors.2 | Selenbp1 | 3.10E−19 |
| Epithelial_Progenitors.2 | Sgpp2 | 7.17E−19 |
| Epithelial_Progenitors.2 | Id1 | 1.45E−18 |
| Epithelial_Progenitors.2 | Gpatch2 | 4.15E−18 |
| Epithelial_Progenitors.2 | Nbeal1 | 4.51E−18 |
| Epithelial_Progenitors.2 | Ildr1 | 4.64E−18 |
| Epithelial_Progenitors.2 | Dsc2 | 2.59E−17 |
| Epithelial_Progenitors.2 | Car2 | 2.82E−17 |
| Epithelial_Progenitors.2 | Tnfrsf11a | 3.73E−17 |
| Epithelial_Progenitors.2 | Kitl | 3.79E−17 |
| Epithelial_Progenitors.2 | Pard3b | 4.18E−17 |
| Epithelial_Progenitors.2 | Vsig10 | 4.66E−17 |
| Epithelial_Progenitors.2 | Ppara | 7.65E−17 |
| Epithelial_Progenitors.2 | Ppargc1b | 8.80E−17 |
| Epithelial_Progenitors.2 | Cyp2c65 | 1.29E−16 |
| Epithelial_Progenitors.2 | Sema5a | 1.39E−16 |
| Epithelial_Progenitors.2 | Lgals3 | 3.52E−16 |
| Epithelial_Progenitors.2 | Ap3b1 | 3.93E−16 |
| Epithelial_Progenitors.2 | Plce1 | 3.94E−16 |
| Epithelial_Progenitors.2 | Lurap1l | 4.74E−16 |
| Epithelial_Progenitors.2 | Lgals4 | 2.73E−15 |
| Epithelial_Progenitors.2 | Pycard | 3.14E−15 |
| Epithelial_Progenitors.2 | Pbx1 | 3.21E−15 |
| Epithelial_Progenitors.2 | Nr3c2 | 3.34E−15 |
| Epithelial_Progenitors.2 | Slc4a4 | 5.54E−15 |
| Epithelial_Progenitors.2 | Akr1c19 | 8.28E−15 |
| Epithelial_Progenitors.2 | Ppp1r9a | 1.45E−14 |
| Epithelial_Progenitors.2 | Ppard | 2.28E−14 |
| Epithelial_Progenitors.2 | Sema3c | 2.81E−14 |
| Epithelial_Progenitors.2 | Palld | 3.22E−14 |
| Epithelial_Progenitors.2 | Samd12 | 3.92E−14 |
| Epithelial_Progenitors.2 | Ces1f | 7.39E−14 |
| Epithelial_Progenitors.2 | Prkca | 9.52E−14 |
| Epithelial_Progenitors.2 | Snx13 | 1.02E−13 |
| Epithelial_Progenitors.2 | Ank | 1.27E−13 |
| Epithelial_Progenitors.2 | Zfp618 | 3.34E−13 |
| Epithelial_Progenitors.2 | Tex9 | 4.66E−13 |
| Epithelial_Progenitors.2 | Pigr | 1.12E−12 |
| Epithelial_Progenitors.2 | Ccl28 | 1.21E−12 |
| Epithelial_Progenitors.2 | Fa2h | 2.42E−12 |
| Epithelial_Progenitors.2 | Tubal3 | 1.31E−10 |
| Epithelial_Progenitors.2 | Pitpnm3 | 1.64E−10 |
| Epithelial_Progenitors.2 | Cldn8 | 2.67E−09 |
| Epithelial_Progenitors.2 | Pbld2 | 3.54E−09 |
| Epithelial_Progenitors.2 | Utp20 | 4.69E−09 |
| Epithelial_Progenitors.2 | 9130008F23Rik | 4.99E−09 |
| Epithelial_Progenitors.2 | Trabd2b | 8.74E−09 |
| Epithelial_Progenitors.2 | Prelid2 | 1.00E−08 |
| Epithelial_Progenitors.2 | 4430402I18Rik | 2.03E−08 |
| Epithelial_Progenitors.2 | Slc22a18 | 5.33E−08 |
| Epithelial_Progenitors.2 | Prkg2 | 6.37E−08 |
| Epithelial_Progenitors.2 | Vstm5 | 2.06E−07 |
| Epithelial_Progenitors.2 | Spata17 | 3.29E−07 |
| Epithelial_Progenitors.2 | Trim16 | 3.32E−07 |
| Epithelial_Progenitors.2 | Cyp2c68 | 3.92E−07 |
| Epithelial_Progenitors.2 | Ugt2b5 | 4.30E−07 |
| Epithelial_Progenitors.2 | Gm10399 | 4.30E−07 |
| Epithelial_Progenitors.2 | Acsm3 | 4.32E−07 |
| Epithelial_Progenitors.2 | Tbc1d2 | 5.65E−07 |
| Epithelial_Progenitors.2 | E230001N04Rik | 7.65E−07 |
| Epithelial_Progenitors.2 | Dnah8 | 1.06E−06 |
| Epithelial_Progenitors.2 | Nqo1 | 1.28E−06 |
| Epithelial_Progenitors.2 | Dgkg | 1.37E−06 |
| Epithelial_Progenitors.2 | Cwh43 | 1.74E−06 |
| Epithelial_Progenitors.2 | Il18 | 2.16E−06 |
| Epithelial_Progenitors.2 | Slc16a9 | 2.56E−06 |
| Epithelial_Progenitors.2 | Sult1b1 | 3.73E−06 |
| Epithelial_Progenitors.2 | Lancl3 | 4.47E−06 |
| Epithelial_Progenitors.2 | Zfp697 | 6.14E−06 |
| Epithelial_Progenitors.2 | Aadac | 7.08E−06 |
| Epithelial_Progenitors.2 | Hunk | 7.24E−06 |
| Epithelial_Progenitors.2 | Cmbl | 8.86E−06 |
| Epithelial_Progenitors.2 | Nceh1 | 4.17E−05 |
| Epithelial_Progenitors.2 | Rnf152 | 4.40E−05 |
| Epithelial_Progenitors.2 | Pgd | 4.43E−05 |
| Epithelial_Progenitors.2 | Gm43824 | 4.49E−05 |
| Epithelial_Progenitors.2 | Smad6 | 8.40E−05 |
| Epithelial_Progenitors.2 | Slc39a5 | 1.00E−04 |
| Epithelial_Progenitors.2 | Maob | 1.16E−04 |
| Epithelial_Progenitors.2 | Tlr1 | 1.16E−04 |
| Epithelial_Progenitors.2 | Gpr160 | 1.38E−04 |
| Epithelial_Progenitors.2 | Glod5 | 4.05E−04 |
| Epithelial_Progenitors.2 | Cyp4f40 | 4.14E−04 |
| Epithelial_Progenitors.2 | Jag1 | 4.14E−04 |
| Epithelial_Progenitors.2 | Sh3rf2 | 8.56E−04 |
| Epithelial_Progenitors.2 | Sobp | 1.25E−03 |
| Epithelial_Progenitors.2 | Rassf9 | 1.42E−03 |
| Epithelial_Progenitors.2 | Rgp1 | 1.53E−03 |
| Epithelial_Progenitors.2 | Fzd5 | 1.83E−03 |
| Epithelial_Progenitors.2 | Slc16a5 | 2.23E−03 |
| Epithelial_Progenitors.2 | Fahd1 | 2.99E−03 |
| Epithelial_Progenitors.2 | S100b | 3.02E−03 |
| Epithelial_Progenitors.2 | Mypop | 3.21E−03 |
| Epithelial_Progenitors.2 | Pm20d1 | 4.05E−03 |
| Epithelial_Progenitors.2 | E2f5 | 4.32E−03 |
| Epithelial_Progenitors.2 | Mst1r | 4.56E−03 |
| Epithelial_Progenitors.2 | Arl14 | 4.98E−03 |
| Epithelial_Progenitors.2 | Ddx43 | 5.64E−03 |
| Epithelial_Progenitors.2 | Gm15884 | 6.31E−03 |
| Epithelial_Progenitors.2 | Adap1 | 7.97E−03 |
| Epithelial_Progenitors.2 | Gm12576 | 3.23E−02 |
| Fibroblast | Celf2 | 2.95E−111 |
| Fibroblast | Lama2 | 9.21E−111 |
| Fibroblast | Pcdh7 | 2.34E−108 |
| Fibroblast | Col3a1 | 2.98E−97 |
| Fibroblast | Pid1 | 1.12E−91 |
| Fibroblast | Sdk1 | 1.67E−86 |
| Fibroblast | Dlc1 | 3.26E−84 |
| Fibroblast | Tenm3 | 4.02E−82 |
| Fibroblast | Zeb2 | 1.40E−76 |
| Fibroblast | Robo2 | 3.67E−76 |
| Fibroblast | Bmp5 | 1.77E−70 |
| Fibroblast | Col5a2 | 2.09E−70 |
| Fibroblast | Slit3 | 8.92E−66 |
| Fibroblast | Tgfbr3 | 1.60E−64 |
| Fibroblast | Gpc6 | 1.27E−59 |
| Fibroblast | Adamdec1 | 3.41E−59 |
| Fibroblast | Col1a2 | 2.45E−55 |
| Fibroblast | Robo1 | 1.63E−53 |
| Fibroblast | Sox5 | 4.81E−53 |
| Fibroblast | Col6a3 | 4.13E−52 |
| Fibroblast | Ghr | 7.14E−52 |
| Fibroblast | Rora | 1.19E−50 |
| Fibroblast | Bicc1 | 1.86E−50 |
| Fibroblast | Dcn | 1.67E−48 |
| Fibroblast | Pdgfra | 3.50E−47 |
| Fibroblast | Malat1 | 9.73E−47 |
| Fibroblast | Cped1 | 1.43E−45 |
| Fibroblast | Rad51b | 2.72E−45 |
| Fibroblast | Negr1 | 9.63E−45 |
| Fibroblast | Adgrl3 | 4.12E−44 |
| Fibroblast | Tnc | 4.04E−42 |
| Fibroblast | Tshz2 | 1.33E−40 |
| Fibroblast | Dnm3 | 1.99E−40 |
| Fibroblast | Fndc1 | 9.05E−40 |
| Fibroblast | Zbtb20 | 1.20E−39 |
| Fibroblast | Efemp1 | 5.04E−39 |
| Fibroblast | Abi3bp | 1.03E−38 |
| Fibroblast | Pde1a | 2.31E−37 |
| Fibroblast | Rbms3 | 2.36E−37 |
| Fibroblast | Zbtb16 | 6.20E−37 |
| Fibroblast | Postn | 1.04E−36 |
| Fibroblast | Magi2 | 1.68E−35 |
| Fibroblast | Pappa | 1.84E−35 |
| Fibroblast | Ext1 | 3.09E−35 |
| Fibroblast | Bmp4 | 1.29E−34 |
| Fibroblast | Fbn1 | 1.74E−34 |
| Fibroblast | Bmp6 | 3.77E−34 |
| Fibroblast | Arhgap6 | 3.86E−34 |
| Fibroblast | Dpt | 5.92E−34 |
| Fibroblast | Fstl1 | 7.65E−34 |
| Fibroblast | Pcdh9 | 1.27E−33 |
| Fibroblast | Col1a1 | 3.43E−33 |
| Fibroblast | Tcf4 | 4.30E−33 |
| Fibroblast | Pdzrn3 | 1.50E−32 |
| Fibroblast | Zfpm2 | 2.78E−32 |
| Fibroblast | Chsy3 | 3.29E−32 |
| Fibroblast | Fmnl2 | 3.40E−32 |
| Fibroblast | Hmcn2 | 8.92E−32 |
| Fibroblast | Prr16 | 1.27E−31 |
| Fibroblast | Sparc | 1.54E−31 |
| Fibroblast | Arhgap10 | 4.01E−31 |
| Fibroblast | Aspn | 6.11E−31 |
| Fibroblast | 9530026P05Rik | 9.51E−31 |
| Fibroblast | Bnc2 | 1.24E−30 |
| Fibroblast | Prickle1 | 1.95E−30 |
| Fibroblast | Fbx17 | 2.88E−30 |
| Fibroblast | Igfbp7 | 3.48E−30 |
| Fibroblast | Lamc1 | 4.59E−30 |
| Fibroblast | Rhoj | 9.29E−30 |
| Fibroblast | Ebf1 | 1.51E−29 |
| Fibroblast | Meis2 | 1.55E−29 |
| Fibroblast | Gsn | 4.64E−29 |
| Fibroblast | Rbpms | 5.71E−29 |
| Fibroblast | Eln | 1.02E−28 |
| Fibroblast | Gli3 | 2.55E−28 |
| Fibroblast | Nav1 | 5.31E−28 |
| Fibroblast | Zeb1 | 7.36E−28 |
| Fibroblast | Svep1 | 8.37E−28 |
| Fibroblast | Col6a1 | 1.29E−27 |
| Fibroblast | Lhfp | 1.52E−27 |
| Fibroblast | Ncam1 | 1.74E−27 |
| Fibroblast | Serping1 | 1.77E−27 |
| Fibroblast | Col6a2 | 2.04E−27 |
| Fibroblast | Fbln1 | 3.36E−27 |
| Fibroblast | Nckap5 | 1.03E−26 |
| Fibroblast | Ddr2 | 4.55E−26 |
| Fibroblast | Meg3 | 8.59E−26 |
| Fibroblast | Rbms1 | 1.16E−25 |
| Fibroblast | Gm26719 | 3.82E−25 |
| Fibroblast | Vcan | 5.59E−25 |
| Fibroblast | Ldlrad4 | 9.33E−25 |
| Fibroblast | Mast4 | 1.96E−24 |
| Fibroblast | Abca8a | 3.72E−24 |
| Fibroblast | Meis1 | 9.19E−24 |
| Fibroblast | Tcf21 | 2.30E−23 |
| Fibroblast | Cald1 | 3.06E−23 |
| Fibroblast | Lama4 | 3.45E−23 |
| Fibroblast | Akt3 | 3.77E−23 |
| Fibroblast | Prickle2 | 4.55E−23 |
| Fibroblast | Arhgap28 | 1.34E−22 |
| Fibroblast | Bgn | 1.13E−21 |
| Fibroblast | Ccdc80 | 2.64E−21 |
| Fibroblast | Axl | 2.92E−21 |
| Fibroblast | Lamb1 | 2.23E−20 |
| Fibroblast | Htra3 | 3.61E−19 |
| Fibroblast | Serpinh1 | 9.76E−19 |
| Fibroblast | Mmp2 | 1.41E−18 |
| Fibroblast | Pla2r1 | 1.62E−18 |
| Fibroblast | 4930467D21Rik | 1.95E−18 |
| Fibroblast | Spon2 | 2.65E−18 |
| Fibroblast | Fam198b | 9.64E−17 |
| Fibroblast | Tshz3 | 2.36E−16 |
| Fibroblast | Lum | 6.68E−16 |
| Fibroblast | Clec3b | 6.70E−16 |
| Fibroblast | Scube1 | 1.03E−15 |
| Fibroblast | Ptgs1 | 2.70E−15 |
| Fibroblast | Mfap5 | 8.75E−15 |
| Fibroblast | Hgf | 9.90E−15 |
| Fibroblast | Dnm3os | 1.27E−14 |
| Fibroblast | Emid1 | 3.83E−14 |
| Fibroblast | Cxcl12 | 5.76E−14 |
| Fibroblast | Pi16 | 6.28E−14 |
| Fibroblast | Bdkrb2 | 1.11E−13 |
| Fibroblast | Fam20a | 1.76E−13 |
| Fibroblast | Tmem119 | 1.99E−13 |
| Fibroblast | Gm42532 | 2.16E−13 |
| Fibroblast | Mgp | 2.16E−13 |
| Fibroblast | Ednra | 3.59E−13 |
| Fibroblast | Mfap4 | 9.12E−13 |
| Fibroblast | Gli2 | 1.06E−12 |
| Fibroblast | Col15a1 | 1.19E−12 |
| Fibroblast | Cygb | 2.75E−12 |
| Fibroblast | Col4a6 | 5.07E−12 |
| Fibroblast | Nova1 | 5.09E−12 |
| Fibroblast | Col24a1 | 1.06E−11 |
| Fibroblast | Srpx2 | 9.65E−11 |
| Fibroblast | Cilp | 1.55E−10 |
| Fibroblast | Ms4a4d | 1.63E−10 |
| Fibroblast | Ereg | 1.98E−10 |
| Fibroblast | Cml3 | 6.21E−09 |
| Fibroblast | Pcolce | 7.25E−09 |
| Fibroblast | Ednrb | 1.08E−08 |
| Fibroblast | Olfml2b | 4.37E−08 |
| Fibroblast | Sfrp1 | 1.93E−07 |
| Fibroblast | Jam2 | 2.36E−07 |
| Fibroblast | Lama1 | 3.83E−07 |
| Fibroblast | Naa11 | 4.26E−07 |
| Fibroblast | Enpp2 | 4.31E−07 |
| Fibroblast | Podn | 4.33E−07 |
| Fibroblast | Col5a3 | 6.36E−07 |
| Fibroblast | Adamts5 | 8.81E−07 |
| Fibroblast | Clqtnf7 | 1.40E−06 |
| Fibroblast | Cyp7b1 | 1.64E−06 |
| Fibroblast | Prkcdbp | 2.99E−06 |
| Fibroblast | Syt13 | 5.85E−06 |
| Glia | Cdh19 | 9.31E−191 |
| Glia | Nkain2 | 5.05E−184 |
| Glia | Slc35f1 | 2.42E−180 |
| Glia | Ncam1 | 4.01E−145 |
| Glia | Ptprz1 | 2.38E−144 |
| Glia | Grik2 | 1.52E−133 |
| Glia | Ppp2r2b | 6.24E−129 |
| Glia | Kcnq5 | 5.36E−124 |
| Glia | Dtna | 1.04E−121 |
| Glia | Lrrc4c | 1.63E−110 |
| Glia | Sorcs1 | 1.74E−107 |
| Glia | Ank3 | 4.51E−105 |
| Glia | Rora | 5.26E−103 |
| Glia | Col11a1 | 1.49E−102 |
| Glia | Plce1 | 2.77E−102 |
| Glia | Il1rapl1 | 4.82E−95 |
| Glia | Sntb1 | 5.57E−93 |
| Glia | Adam23 | 9.22E−90 |
| Glia | Adgrl3 | 1.48E−89 |
| Glia | Zeb2 | 1.83E−88 |
| Glia | Sgip1 | 2.81E−87 |
| Glia | Cdh2 | 8.15E−86 |
| Glia | Plcb1 | 5.43E−81 |
| Glia | Scn7a | 8.03E−80 |
| Glia | Col12a1 | 1.48E−79 |
| Glia | Etl4 | 2.23E−79 |
| Glia | Gfra1 | 2.36E−79 |
| Glia | Tgfb2 | 5.69E−77 |
| Glia | Csmd1 | 1.01E−75 |
| Glia | Adam11 | 3.26E−74 |
| Glia | Glp2r | 4.42E−73 |
| Glia | Dmd | 4.13E−72 |
| Glia | Sox5 | 2.44E−71 |
| Glia | Ncam2 | 2.59E−70 |
| Glia | Kif21a | 3.60E−70 |
| Glia | Sorbs1 | 1.69E−67 |
| Glia | Pmepa1 | 1.83E−67 |
| Glia | Hmcn1 | 1.83E−67 |
| Glia | Chl1 | 4.51E−67 |
| Glia | Qk | 2.37E−64 |
| Glia | Sox10 | 8.41E−64 |
| Glia | Nrg3 | 1.24E−63 |
| Glia | Dock10 | 1.44E−62 |
| Glia | Dlgap1 | 1.72E−62 |
| Glia | Lsamp | 4.02E−62 |
| Glia | Agmo | 1.40E−60 |
| Glia | Tmprss5 | 6.64E−59 |
| Glia | Ctnna3 | 2.00E−58 |
| Glia | Ltbp1 | 2.19E−58 |
| Glia | Zfp536 | 3.40E−57 |
| Glia | Igsf11 | 4.05E−56 |
| Glia | Sparc | 2.78E−55 |
| Glia | Erc2 | 3.88E−54 |
| Glia | Col18a1 | 5.37E−54 |
| Glia | Art3 | 8.85E−54 |
| Glia | Grb14 | 1.13E−53 |
| Glia | Fign | 1.41E−52 |
| Glia | Zbtb20 | 1.89E−52 |
| Glia | Pde7b | 2.18E−52 |
| Glia | Lpar1 | 2.18E−52 |
| Glia | Sorbs2 | 4.10E−52 |
| Glia | Ank2 | 9.14E−52 |
| Glia | Ggta1 | 2.68E−51 |
| Glia | Ldlrad4 | 6.70E−51 |
| Glia | Gpcpd1 | 7.86E−51 |
| Glia | Malat1 | 9.66E−51 |
| Glia | Lrrtm3 | 1.21E−49 |
| Glia | Sema3e | 1.95E−48 |
| Glia | Tbx3os1 | 2.55E−48 |
| Glia | Sgcd | 3.39E−48 |
| Glia | P3h2 | 1.04E−47 |
| Glia | Agbl4 | 1.22E−46 |
| Glia | Apoe | 3.14E−46 |
| Glia | Klhl29 | 9.22E−46 |
| Glia | Atp1a2 | 1.32E−45 |
| Glia | Zfhx4 | 6.25E−45 |
| Glia | Prkg1 | 1.69E−44 |
| Glia | Gm10863 | 5.06E−43 |
| Glia | Pdzd2 | 1.34E−42 |
| Glia | Stk32a | 2.06E−42 |
| Glia | Fxyd1 | 4.68E−42 |
| Glia | Dst | 6.15E−42 |
| Glia | Abca8a | 1.02E−41 |
| Glia | Lgi4 | 1.44E−41 |
| Glia | Efna5 | 1.86E−41 |
| Glia | Ablim2 | 3.69E−41 |
| Glia | Tanc2 | 4.20E−41 |
| Glia | Piezo2 | 5.91E−41 |
| Glia | Adam12 | 6.33E−41 |
| Glia | Gm38505 | 7.40E−41 |
| Glia | Adarb2 | 1.64E−40 |
| Glia | Plxdc2 | 3.30E−40 |
| Glia | Celf2 | 7.79E−40 |
| Glia | Zeb1 | 1.65E−39 |
| Glia | Plxna4 | 4.91E−38 |
| Glia | Fam184b | 7.20E−38 |
| Glia | Ldb2 | 3.07E−37 |
| Glia | Limch1 | 2.43E−36 |
| Glia | Edil3 | 4.73E−36 |
| Glia | Gpam | 1.22E−35 |
| Glia | Rerg | 6.40E−34 |
| Glia | Gpm6b | 4.06E−33 |
| Glia | Plp1 | 7.87E−33 |
| Glia | Pxdn | 3.60E−32 |
| Glia | Shc4 | 2.10E−31 |
| Glia | Hand2 | 7.95E−31 |
| Glia | Fam19a5 | 8.13E−31 |
| Glia | Astn1 | 6.42E−30 |
| Glia | Abca8b | 1.65E−29 |
| Glia | Kcna1 | 4.26E−29 |
| Glia | Armc2 | 5.27E−29 |
| Glia | S1pr3 | 7.62E−27 |
| Glia | Cd59a | 1.12E−26 |
| Glia | Gfap | 8.52E−26 |
| Glia | Gpr37l1 | 1.17E−25 |
| Glia | Olfml2a | 9.82E−25 |
| Glia | Ctgf | 1.07E−24 |
| Glia | Mest | 4.56E−24 |
| Glia | Kcna6 | 5.82E−24 |
| Glia | Gm11099 | 2.22E−22 |
| Glia | Nme5 | 1.48E−20 |
| Glia | Cmtm5 | 2.96E−19 |
| Glia | Kcna2 | 1.65E−18 |
| Glia | Ttyh1 | 1.96E−18 |
| Glia | Gjc3 | 3.42E−18 |
| Glia | C130071C03Rik | 3.43E−18 |
| Glia | Snca | 7.80E−18 |
| Glia | Islr | 8.95E−18 |
| Glia | Itgb8 | 1.47E−15 |
| Glia | Lrrn2 | 2.00E−15 |
| Glia | Gfra2 | 3.32E−15 |
| Glia | Kcnip3 | 1.01E−14 |
| Glia | Scrn1 | 1.28E−14 |
| Glia | P4ha3 | 2.19E−14 |
| Glia | Col9a2 | 4.27E−14 |
| Glia | Pdgfb | 5.44E−14 |
| Glia | Plxnb3 | 3.20E−13 |
| Glia | Frzb | 5.61E−13 |
| Glia | Lrriq1 | 6.28E−13 |
| Glia | Hey2 | 9.19E−13 |
| Glia | Sostdc1 | 6.03E−12 |
| Glia | Slitrk6 | 6.03E−12 |
| Glia | Kcnj10 | 7.57E−12 |
| Glia | Drc1 | 1.03E−11 |
| Glia | Srcin1 | 1.75E−11 |
| Glia | Gm12688 | 1.97E−11 |
| Glia | 9630001P10Rik | 2.90E−11 |
| Glia | Stk33 | 3.61E−11 |
| Glia | Gm11149 | 4.36E−11 |
| Glia | Gm37679 | 5.26E−11 |
| Glia | Olfml3 | 8.85E−11 |
| Glia | A630012P03Rik | 9.69E−11 |
| Glia | Gm20726 | 1.60E−10 |
| Glia | Fam107a | 3.90E−10 |
| Glia | Gm4477 | 8.31E−10 |
| Glia | Iqub | 4.00E−09 |
| Glia | Sdc3 | 5.60E−09 |
| Glia | Lrrc9 | 7.04E−09 |
| Glia | Rsph10b | 1.80E−08 |
| Glia | Atp1b2 | 2.20E−08 |
| Glia | E530001K10Rik | 2.82E−08 |
| Glia | Hoxc4 | 7.90E−08 |
| Glia | Paqr6 | 8.95E−08 |
| Glia | Crtac1 | 1.12E−07 |
| Glia | Vstm4 | 1.15E−07 |
| Glia | Cfap44 | 1.52E−07 |
| Glia | Kcnj12 | 2.66E−07 |
| Glia | Tub | 1.38E−06 |
| Goblet | Fcgbp | 0.00E+00 |
| Goblet | Zg16 | 1.62E−294 |
| Goblet | Clca1 | 2.77E−272 |
| Goblet | Fer1l6 | 3.18E−200 |
| Goblet | Clec2h | 2.10E−158 |
| Goblet | Muc2 | 2.80E−148 |
| Goblet | Bcas1 | 5.45E−143 |
| Goblet | Sval1 | 1.19E−138 |
| Goblet | Tff3 | 5.43E−134 |
| Goblet | Sytl2 | 6.33E−125 |
| Goblet | Rab27b | 1.18E−108 |
| Goblet | Rep15 | 4.88E−107 |
| Goblet | Spink1 | 5.64E−106 |
| Goblet | Scin | 1.28E−98 |
| Goblet | Hepacam2 | 1.52E−97 |
| Goblet | Rab27a | 1.61E−92 |
| Goblet | Nr3c2 | 1.33E−85 |
| Goblet | Myo5c | 1.07E−76 |
| Goblet | Hsd11b2 | 7.50E−75 |
| Goblet | Lgals4 | 1.33E−71 |
| Goblet | Kcnma1 | 1.45E−71 |
| Goblet | Tnfaip8 | 1.06E−70 |
| Goblet | Pla2g10os | 1.59E−70 |
| Goblet | Klf4 | 6.15E−68 |
| Goblet | Inpp4b | 3.60E−67 |
| Goblet | Mcf2l | 3.36E−66 |
| Goblet | St6gal1 | 8.77E−64 |
| Goblet | Srgap1 | 6.67E−62 |
| Goblet | Gm12511 | 3.01E−59 |
| Goblet | Mlph | 7.21E−56 |
| Goblet | Cyp2d34 | 2.46E−55 |
| Goblet | Shroom3 | 1.08E−54 |
| Goblet | Ptprn2 | 2.32E−54 |
| Goblet | Nupr1 | 2.95E−54 |
| Goblet | Slfn4 | 1.55E−53 |
| Goblet | Ceacam1 | 4.01E−52 |
| Goblet | Muc13 | 4.01E−52 |
| Goblet | Lypd8 | 6.35E−52 |
| Goblet | Il13ra1 | 6.54E−51 |
| Goblet | Pde4d | 6.69E−51 |
| Goblet | AW112010 | 9.37E−51 |
| Goblet | Neat1 | 1.78E−50 |
| Goblet | Galnt7 | 8.53E−50 |
| Goblet | Ms4a8a | 1.45E−46 |
| Goblet | Capn9 | 1.88E−46 |
| Goblet | Krt8 | 2.73E−46 |
| Goblet | 9030622O22Rik | 3.04E−46 |
| Goblet | P2rx4 | 5.27E−46 |
| Goblet | Ang4 | 6.97E−46 |
| Goblet | Plcb1 | 9.43E−46 |
| Goblet | 2610035D17Rik | 1.11E−43 |
| Goblet | Plcl2 | 2.86E−43 |
| Goblet | Atp2c2 | 3.87E−41 |
| Goblet | Krt18 | 5.86E−41 |
| Goblet | Bace2 | 9.35E−41 |
| Goblet | Ptprr | 6.66E−38 |
| Goblet | Atp8a1 | 8.79E−38 |
| Goblet | Agr2 | 3.12E−37 |
| Goblet | Smim6 | 5.25E−37 |
| Goblet | Slc4a7 | 2.39E−36 |
| Goblet | Cpd | 2.17E−35 |
| Goblet | Trim25 | 8.09E−35 |
| Goblet | Cdc42ep3 | 1.20E−34 |
| Goblet | Ffar4 | 1.73E−34 |
| Goblet | Clmn | 2.05E−34 |
| Goblet | Stxbp1 | 3.14E−34 |
| Goblet | Anxa3 | 6.14E−34 |
| Goblet | Fut8 | 1.79E−33 |
| Goblet | Ccl6 | 2.32E−32 |
| Goblet | S100a6 | 3.21E−32 |
| Goblet | Gcnt3 | 3.25E−32 |
| Goblet | Atoh1 | 3.54E−32 |
| Goblet | Fmn1 | 3.54E−32 |
| Goblet | Tcf7l2 | 4.18E−32 |
| Goblet | Dennd1b | 2.25E−31 |
| Goblet | Ano7 | 2.62E−31 |
| Goblet | Slc22a23 | 5.35E−31 |
| Goblet | Iqgap2 | 2.83E−30 |
| Goblet | Phgr1 | 1.06E−29 |
| Goblet | Mctp2 | 1.80E−29 |
| Goblet | Cdkn1a | 4.28E−29 |
| Goblet | Camk2n1 | 7.61E−29 |
| Goblet | Txn1 | 2.82E−28 |
| Goblet | Gna14 | 4.42E−28 |
| Goblet | Grpr | 5.15E−28 |
| Goblet | Tfcp2l1 | 7.84E−28 |
| Goblet | Lgals9 | 1.27E−27 |
| Goblet | Pld1 | 2.32E−27 |
| Goblet | Tmco3 | 2.93E−27 |
| Goblet | Syt7 | 3.46E−27 |
| Goblet | Baiap2l1 | 7.02E−27 |
| Goblet | Atrnl1 | 8.91E−27 |
| Goblet | Id3 | 1.07E−26 |
| Goblet | Trp53inp1 | 1.95E−25 |
| Goblet | Tsc22d1 | 2.49E−25 |
| Goblet | Galnt10 | 3.38E−25 |
| Goblet | Ghr | 3.38E−25 |
| Goblet | Gm1123 | 3.38E−25 |
| Goblet | Qsox1 | 5.71E−25 |
| Goblet | Dopey2 | 5.96E−25 |
| Goblet | F3 | 4.28E−24 |
| Goblet | Clic4 | 5.00E−24 |
| Goblet | Mfsd7a | 9.31E−24 |
| Goblet | Mptx1 | 5.30E−23 |
| Goblet | Rasa4 | 7.63E−22 |
| Goblet | Ddx60 | 9.67E−22 |
| Goblet | Muc4 | 1.93E−21 |
| Goblet | Frmd3 | 2.37E−21 |
| Goblet | Capn5 | 1.71E−20 |
| Goblet | 2210011C24Rik | 4.73E−20 |
| Goblet | Gde1 | 9.46E−20 |
| Goblet | Entpd8 | 1.30E−19 |
| Goblet | Pkhd1 | 2.10E−19 |
| Goblet | Best2 | 6.95E−19 |
| Goblet | Gm6086 | 1.28E−18 |
| Goblet | Scnn1b | 2.73E−18 |
| Goblet | Fhl2 | 2.85E−18 |
| Goblet | Cmtm8 | 8.91E−18 |
| Goblet | Spats2l | 1.48E−17 |
| Goblet | Tpsg1 | 4.86E−17 |
| Goblet | Samd8 | 1.24E−15 |
| Goblet | Cldn4 | 4.50E−15 |
| Goblet | Apobec1 | 7.24E−15 |
| Goblet | Gnb5 | 9.59E−15 |
| Goblet | Smim5 | 1.71E−14 |
| Goblet | Sytl5 | 2.08E−14 |
| Goblet | Fam117a | 4.55E−13 |
| Goblet | Tor3a | 4.39E−12 |
| Goblet | Rasd2 | 6.97E−12 |
| Goblet | Rasd1 | 8.74E−12 |
| Goblet | Sytl4 | 1.12E−11 |
| Goblet | Zfp664 | 5.99E−11 |
| Goblet | Cyp2d12 | 6.59E−11 |
| Goblet | Gpr20 | 1.09E−10 |
| Goblet | Gm9994 | 6.81E−10 |
| Goblet | Bcas1os2 | 1.06E−08 |
| Goblet | Tmc1 | 3.56E−08 |
| Goblet | Ttc39aos1 | 9.56E−08 |
| Goblet | Pla2g2c | 1.02E−07 |
| Goblet | Kcnf1 | 7.36E−07 |
| Goblet | Slc23a3 | 7.83E−07 |
| Goblet | Upk1a | 3.72E−06 |
| Goblet | Ubxn10 | 7.02E−06 |
| Goblet | Gm28588 | 1.13E−05 |
| Goblet | Dhrs9 | 1.25E−05 |
| Goblet | Nlrp4e | 1.65E−05 |
| Goblet | Spire1 | 5.13E−05 |
| Goblet | Slc2a10 | 7.55E−05 |
| Goblet | Oasl1 | 1.59E−04 |
| Goblet | Atp12a | 2.72E−04 |
| Goblet | Cacna2d2 | 3.49E−04 |
| Goblet | Grin1 | 7.37E−04 |
| Goblet | Sct | 1.54E−03 |
| Goblet | Gm15658 | 4.63E−03 |
| Macrophage | Rbpj | 9.28E−69 |
| Macrophage | Zeb2 | 1.25E−62 |
| Macrophage | Slc9a9 | 3.94E−62 |
| Macrophage | Ms4a6c | 7.02E−53 |
| Macrophage | Arhgap15 | 6.77E−50 |
| Macrophage | Mrc1 | 1.70E−48 |
| Macrophage | F13a1 | 1.04E−46 |
| Macrophage | Pid1 | 2.40E−46 |
| Macrophage | F630028O10Rik | 1.98E−45 |
| Macrophage | Trps1 | 1.47E−44 |
| Macrophage | Fyb | 2.17E−43 |
| Macrophage | Dab2 | 1.63E−40 |
| Macrophage | Adgre1 | 3.19E−40 |
| Macrophage | H2-Eb1 | 2.17E−36 |
| Macrophage | Stab1 | 5.09E−36 |
| Macrophage | Myo1f | 1.68E−35 |
| Macrophage | Ctsc | 1.12E−34 |
| Macrophage | Lyz2 | 8.00E−34 |
| Macrophage | Cd74 | 2.50E−33 |
| Macrophage | Pip4k2a | 1.02E−31 |
| Macrophage | Inpp5d | 3.86E−31 |
| Macrophage | Gm26740 | 2.74E−30 |
| Macrophage | Hmha1 | 3.55E−30 |
| Macrophage | C1qc | 8.29E−30 |
| Macrophage | Mir142hg | 6.85E−29 |
| Macrophage | Aoah | 7.30E−29 |
| Macrophage | Fam105a | 1.37E−28 |
| Macrophage | Ms4a6b | 1.27E−27 |
| Macrophage | Ptprc | 2.81E−27 |
| Macrophage | Abcg3 | 5.10E−27 |
| Macrophage | Csf1r | 5.19E−27 |
| Macrophage | Dock10 | 5.59E−27 |
| Macrophage | Lyn | 1.42E−26 |
| Macrophage | Spi1 | 8.79E−26 |
| Macrophage | Ms4a4a | 1.14E−25 |
| Macrophage | Lcp1 | 3.84E−25 |
| Macrophage | Ly86 | 3.85E−25 |
| Macrophage | P2ry6 | 8.33E−25 |
| Macrophage | C1qb | 2.58E−24 |
| Macrophage | Cd84 | 5.13E−24 |
| Macrophage | Gab2 | 5.13E−24 |
| Macrophage | Cd163 | 9.79E−24 |
| Macrophage | Cd33 | 9.79E−24 |
| Macrophage | Lair1 | 9.79E−24 |
| Macrophage | Pla2g7 | 3.29E−23 |
| Macrophage | Apobec1 | 3.98E−23 |
| Macrophage | Mafb | 9.40E−23 |
| Macrophage | Klra2 | 3.67E−22 |
| Macrophage | H2-Aa | 4.93E−22 |
| Macrophage | Apoe | 7.91E−22 |
| Macrophage | Adam19 | 8.07E−22 |
| Macrophage | Ikzf1 | 1.86E−21 |
| Macrophage | C1qa | 1.86E−21 |
| Macrophage | Maf | 1.86E−21 |
| Macrophage | Fcer1g | 5.10E−21 |
| Macrophage | Mbnl1 | 1.83E−20 |
| Macrophage | Cfh | 2.19E−20 |
| Macrophage | Pf4 | 3.63E−20 |
| Macrophage | AI607873 | 4.07E−20 |
| Macrophage | Unc93b1 | 1.48E−19 |
| Macrophage | Adcy7 | 1.77E−19 |
| Macrophage | March1 | 2.17E−19 |
| Macrophage | Dock2 | 2.50E−19 |
| Macrophage | Mitf | 3.29E−19 |
| Macrophage | Sirpa | 6.07E−19 |
| Macrophage | Mctp1 | 1.01E−18 |
| Macrophage | P2ry12 | 1.01E−18 |
| Macrophage | Tgfbr1 | 1.25E−18 |
| Macrophage | Myo5a | 1.40E−18 |
| Macrophage | Srgap2 | 2.59E−18 |
| Macrophage | Abca9 | 2.79E−18 |
| Macrophage | Adap2os | 2.79E−18 |
| Macrophage | Frmd4b | 2.90E−18 |
| Macrophage | Gm26522 | 3.81E−18 |
| Macrophage | Cd300a | 6.08E−18 |
| Macrophage | Ms4a7 | 1.37E−17 |
| Macrophage | Fli1 | 1.99E−17 |
| Macrophage | Adap2 | 2.04E−17 |
| Macrophage | Fcgr2b | 2.51E−17 |
| Macrophage | Cybb | 6.05E−17 |
| Macrophage | Hpgds | 7.91E−17 |
| Macrophage | Runx1 | 2.60E−16 |
| Macrophage | Lst1 | 2.65E−16 |
| Macrophage | Tbxas1 | 2.82E−16 |
| Macrophage | H2-Ab1 | 3.36E−16 |
| Macrophage | Tmcc3 | 3.44E−16 |
| Macrophage | Clec4a2 | 5.05E−16 |
| Macrophage | Tgfbi | 1.09E−15 |
| Macrophage | Ubash3b | 2.24E−15 |
| Macrophage | Malat1 | 5.90E−15 |
| Macrophage | Wdfy4 | 6.06E−15 |
| Macrophage | Bank1 | 8.83E−15 |
| Macrophage | Abca1 | 1.19E−14 |
| Macrophage | Ccr5 | 1.21E−14 |
| Macrophage | Epsti1 | 1.51E−14 |
| Macrophage | Ptprj | 2.60E−14 |
| Macrophage | Fermt3 | 2.74E−14 |
| Macrophage | Dock4 | 3.28E−14 |
| Macrophage | Rreb1 | 8.35E−14 |
| Macrophage | Hck | 8.55E−14 |
| Macrophage | Lilrb4a | 9.73E−14 |
| Macrophage | Tyrobp | 5.45E−13 |
| Macrophage | Ccr1 | 8.62E−13 |
| Macrophage | C3ar1 | 3.35E−12 |
| Macrophage | Dnase1l3 | 4.58E−12 |
| Macrophage | Gpr141 | 6.83E−12 |
| Macrophage | Havcr2 | 4.68E−11 |
| Macrophage | Cmklr1 | 5.43E−11 |
| Macrophage | Cd86 | 5.77E−11 |
| Macrophage | Arrb2 | 6.84E−11 |
| Macrophage | Ncf1 | 2.05E−10 |
| Macrophage | Gm42747 | 2.47E−10 |
| Macrophage | Lilra5 | 2.47E−10 |
| Macrophage | H2-DMb1 | 4.22E−10 |
| Macrophage | Cyth4 | 6.52E−10 |
| Macrophage | AF251705 | 2.06E−09 |
| Macrophage | Slc11a1 | 2.06E−09 |
| Macrophage | 2010013B24Rik | 2.06E−09 |
| Macrophage | Gpr34 | 2.16E−09 |
| Macrophage | Ntpcr | 2.20E−09 |
| Macrophage | Amz1 | 2.32E−09 |
| Macrophage | Msr1 | 7.48E−09 |
| Macrophage | Itgax | 1.65E−08 |
| Macrophage | Hk3 | 1.65E−08 |
| Macrophage | Adgrg5 | 1.65E−08 |
| Macrophage | Ms4a6d | 1.65E−08 |
| Macrophage | Serpinb8 | 1.65E−08 |
| Macrophage | Nxpe5 | 1.65E−08 |
| Macrophage | Apol7c | 1.65E−08 |
| Macrophage | I830077J02Rik | 2.03E−08 |
| Macrophage | Il10ra | 2.27E−08 |
| Macrophage | Arhgap22 | 5.51E−08 |
| Macrophage | Rasgrp4 | 7.78E−08 |
| Macrophage | Slamf7 | 1.59E−07 |
| Macrophage | Ccr2 | 1.59E−07 |
| Macrophage | 9530059O14Rik | 8.01E−07 |
| Macrophage | H2-DMa | 9.35E−07 |
| Macrophage | Clec4a3 | 1.16E−06 |
| Macrophage | C130050O18Rik | 1.16E−06 |
| Macrophage | Mpeg1 | 1.16E−06 |
| Macrophage | Pik3r6 | 1.17E−06 |
| Macrophage | Ms4a14 | 1.22E−06 |
| Macrophage | Ptafr | 1.43E−06 |
| Macrophage | Cd80 | 3.13E−06 |
| Macrophage | Asb2 | 3.60E−06 |
| Macrophage | Csf2rb | 5.78E−06 |
| Macrophage | Gm30382 | 6.01E−06 |
| Macrophage | Kmo | 6.09E−06 |
| Macrophage | Clec4n | 6.19E−06 |
| Macrophage | Trpm2 | 1.73E−05 |
| Macrophage | Cd4 | 5.89E−05 |
| Macrophage | Retnla | 1.70E−04 |
| Macrophage | Tg | 2.01E−04 |
| Macrophage | Rab3il1 | 2.19E−04 |
| Macrophage | Kcnip3 | 2.47E−04 |
| Macrophage | Slamf8 | 2.50E−04 |
| Macrophage | Fmnl1 | 1.44E−03 |
| Macrophage | Irf5 | 2.23E−03 |
| Macrophage | Arl4c | 2.75E−03 |
| Mesothelial | Dcn | 2.48E−108 |
| Mesothelial | Gpm6a | 1.19E−91 |
| Mesothelial | C3 | 8.52E−90 |
| Mesothelial | Cav1 | 4.47E−86 |
| Mesothelial | Wdr17 | 4.60E−85 |
| Mesothelial | Upk3b | 3.99E−77 |
| Mesothelial | Bnc2 | 5.99E−74 |
| Mesothelial | Gas1 | 2.78E−73 |
| Mesothelial | Cfh | 2.27E−72 |
| Mesothelial | Muc16 | 5.46E−67 |
| Mesothelial | Rspo1 | 3.15E−62 |
| Mesothelial | Efna5 | 6.59E−61 |
| Mesothelial | Plxna4 | 2.77E−60 |
| Mesothelial | Wt1 | 1.68E−59 |
| Mesothelial | Upk1b | 5.31E−58 |
| Mesothelial | Bicd1 | 1.23E−55 |
| Mesothelial | Aebp1 | 8.12E−55 |
| Mesothelial | Fmo2 | 5.85E−54 |
| Mesothelial | Dlc1 | 6.88E−53 |
| Mesothelial | Ptrf | 1.26E−52 |
| Mesothelial | Tmtc1 | 6.21E−52 |
| Mesothelial | Rarres2 | 3.55E−51 |
| Mesothelial | Col3a1 | 4.15E−50 |
| Mesothelial | Celf2 | 1.74E−49 |
| Mesothelial | Igfbp6 | 1.84E−49 |
| Mesothelial | Kcnab1 | 1.71E−47 |
| Mesothelial | Lvrn | 1.86E−47 |
| Mesothelial | Eya4 | 2.41E−47 |
| Mesothelial | Cdon | 2.50E−47 |
| Mesothelial | Slpi | 5.66E−47 |
| Mesothelial | Arhgap29 | 3.13E−46 |
| Mesothelial | Vim | 6.26E−45 |
| Mesothelial | Msln | 8.01E−45 |
| Mesothelial | 9530026P05Rik | 1.31E−44 |
| Mesothelial | Meg3 | 2.28E−44 |
| Mesothelial | Ap4e1 | 4.94E−44 |
| Mesothelial | Sparc | 2.58E−43 |
| Mesothelial | Meis1 | 9.43E−43 |
| Mesothelial | Meis2 | 1.27E−42 |
| Mesothelial | Zfpm2 | 1.78E−42 |
| Mesothelial | Lrrn4 | 8.53E−42 |
| Mesothelial | Wnt5a | 1.76E−39 |
| Mesothelial | Sulf1 | 1.93E−39 |
| Mesothelial | Efemp1 | 2.66E−39 |
| Mesothelial | Adamtsl1 | 6.95E−38 |
| Mesothelial | Pcdh7 | 4.06E−37 |
| Mesothelial | Sox6 | 2.50E−36 |
| Mesothelial | Tmeff2 | 2.61E−36 |
| Mesothelial | Tmem108 | 1.15E−34 |
| Mesothelial | Slit3 | 2.44E−34 |
| Mesothelial | Sdpr | 2.64E−34 |
| Mesothelial | Cdh11 | 3.34E−34 |
| Mesothelial | Spock2 | 5.46E−33 |
| Mesothelial | Timp2 | 6.74E−33 |
| Mesothelial | Oasl2 | 9.33E−33 |
| Mesothelial | Runx1t1 | 1.39E−32 |
| Mesothelial | Cdh3 | 5.93E−31 |
| Mesothelial | Mpp6 | 8.77E−31 |
| Mesothelial | Adam33 | 1.69E−30 |
| Mesothelial | Tgm2 | 3.53E−30 |
| Mesothelial | Col6a1 | 9.33E−30 |
| Mesothelial | Gm20400 | 1.75E−29 |
| Mesothelial | Dab2 | 2.77E−29 |
| Mesothelial | Aldh1a2 | 2.84E−29 |
| Mesothelial | Pde10a | 6.95E−29 |
| Mesothelial | Ar | 1.09E−28 |
| Mesothelial | Col4a5 | 3.71E−28 |
| Mesothelial | Sema3e | 8.97E−28 |
| Mesothelial | Pkhd1l1 | 1.10E−27 |
| Mesothelial | Cd200 | 2.26E−27 |
| Mesothelial | Ptpn13 | 4.34E−27 |
| Mesothelial | Dpp4 | 4.67E−27 |
| Mesothelial | Zbtb16 | 5.04E−27 |
| Mesothelial | Laptm4a | 1.01E−26 |
| Mesothelial | Bicc1 | 1.68E−26 |
| Mesothelial | Tnfrsf11b | 2.05E−26 |
| Mesothelial | Rbbp8 | 2.05E−26 |
| Mesothelial | Serpinh1 | 2.34E−26 |
| Mesothelial | Sema3d | 5.79E−26 |
| Mesothelial | Il6st | 6.85E−26 |
| Mesothelial | Piezo2 | 7.46E−26 |
| Mesothelial | Mmp16 | 1.20E−25 |
| Mesothelial | Ano1 | 1.78E−25 |
| Mesothelial | Gulp1 | 2.14E−25 |
| Mesothelial | Zfhx4 | 2.28E−25 |
| Mesothelial | Ildr2 | 2.70E−25 |
| Mesothelial | Mast4 | 4.23E−25 |
| Mesothelial | Col4a6 | 4.84E−25 |
| Mesothelial | Arhgap28 | 5.20E−25 |
| Mesothelial | Il1rapl1 | 8.88E−25 |
| Mesothelial | Abcb1b | 9.03E−25 |
| Mesothelial | Tm4sf1 | 1.44E−24 |
| Mesothelial | Wt1os | 1.66E−24 |
| Mesothelial | Ahnak | 2.12E−24 |
| Mesothelial | Cald1 | 3.41E−24 |
| Mesothelial | Nbl1 | 4.09E−24 |
| Mesothelial | Vwa3a | 6.41E−24 |
| Mesothelial | Rbpms | 6.45E−24 |
| Mesothelial | Adgrd1 | 1.11E−23 |
| Mesothelial | Ccbe1 | 1.51E−23 |
| Mesothelial | Nxph1 | 1.99E−20 |
| Mesothelial | Cybrd1 | 3.90E−20 |
| Mesothelial | Gpc3 | 6.67E−20 |
| Mesothelial | C1s1 | 1.22E−19 |
| Mesothelial | Gm15581 | 1.67E−18 |
| Mesothelial | Myrf | 2.83E−18 |
| Mesothelial | Fam184a | 4.35E−18 |
| Mesothelial | Gm12381 | 1.31E−17 |
| Mesothelial | Bcam | 5.99E−16 |
| Mesothelial | Stk26 | 1.34E−15 |
| Mesothelial | Fam180a | 2.40E−14 |
| Mesothelial | Podn | 3.86E−14 |
| Mesothelial | Gm765 | 2.37E−13 |
| Mesothelial | Clu | 3.17E−13 |
| Mesothelial | Npr1 | 3.58E−13 |
| Mesothelial | Lrp2 | 1.19E−12 |
| Mesothelial | Osr1 | 2.55E−12 |
| Mesothelial | Krt14 | 2.55E−12 |
| Mesothelial | A330015K06Rik | 2.92E−12 |
| Mesothelial | Medag | 3.42E−11 |
| Mesothelial | Basp1 | 7.62E−11 |
| Mesothelial | Bnc1 | 2.62E−10 |
| Mesothelial | Fmod | 2.62E−10 |
| Mesothelial | Stbd1 | 2.62E−10 |
| Mesothelial | Smtnl2 | 3.09E−10 |
| Mesothelial | Smim1 | 3.24E−10 |
| Mesothelial | Rcn1 | 6.53E−10 |
| Mesothelial | 2600014E21Rik | 1.41E−09 |
| Mesothelial | Prss12 | 1.62E−09 |
| Mesothelial | Zfp185 | 2.06E−09 |
| Mesothelial | Tmem119 | 2.48E−09 |
| Mesothelial | Lox | 2.21E−08 |
| Mesothelial | Tmem255a | 2.24E−08 |
| Mesothelial | Iigp1 | 2.43E−08 |
| Mesothelial | Rpp25 | 1.81E−07 |
| Mesothelial | Esam | 2.08E−07 |
| Mesothelial | Col4a3 | 2.29E−07 |
| Mesothelial | Angptl4 | 2.52E−07 |
| Mesothelial | Crb2 | 2.52E−07 |
| Mesothelial | Lgals7 | 2.52E−07 |
| Mesothelial | Sh3tc2 | 3.91E−07 |
| Mesothelial | Thbd | 4.86E−07 |
| Mesothelial | Tgfb3 | 5.26E−07 |
| Mesothelial | Gjb5 | 8.19E−07 |
| Mesothelial | Vwc2 | 1.66E−06 |
| Mesothelial | Serpinb6b | 2.40E−06 |
| Mesothelial | Sult5a1 | 4.89E−06 |
| Mesothelial | Slc4a3 | 7.26E−06 |
| Mesothelial | Nnmt | 8.09E−06 |
| Mesothelial | Gpr88 | 1.32E−05 |
| Mesothelial | Galntl5 | 1.37E−05 |
| Mesothelial | Steap4 | 2.67E−05 |
| Mesothelial | Il18r1 | 5.47E−05 |
| Mesothelial | Sspn | 5.95E−05 |
| Mesothelial | P2rx6 | 1.07E−04 |
| Mesothelial | Gm4951 | 1.07E−04 |
| Mesothelial | AW551984 | 2.16E−04 |
| Mesothelial | Sox12 | 3.93E−04 |
| Mesothelial | Lrrn4cl | 3.94E−04 |
| Mesothelial | Spon2 | 1.83E−03 |
| Mesothelial | Miat | 2.06E−03 |
| Mesothelial | Gabre | 3.05E−03 |
| Muscle/ICCs | Myh11 | 6.96E−216 |
| Muscle/ICCs | Cacnb2 | 2.71E−210 |
| Muscle/ICCs | Dmd | 3.97E−205 |
| Muscle/ICCs | Prkg1 | 1.42E−201 |
| Muscle/ICCs | Rbpms | 1.42E−192 |
| Muscle/ICCs | Cacna1c | 3.39E−170 |
| Muscle/ICCs | Sntg2 | 5.97E−170 |
| Muscle/ICCs | Synpo2 | 3.80E−166 |
| Muscle/ICCs | Slc24a3 | 4.47E−154 |
| Muscle/ICCs | Cald1 | 5.50E−152 |
| Muscle/ICCs | Kcnip4 | 3.71E−139 |
| Muscle/ICCs | Meis2 | 2.14E−134 |
| Muscle/ICCs | Actg2 | 6.39E−131 |
| Muscle/ICCs | Svil | 2.31E−130 |
| Muscle/ICCs | Flna | 1.85E−129 |
| Muscle/ICCs | Acta2 | 4.19E−128 |
| Muscle/ICCs | Foxp2 | 1.39E−124 |
| Muscle/ICCs | Mir143hg | 1.92E−124 |
| Muscle/ICCs | Mylk | 3.30E−123 |
| Muscle/ICCs | Lpp | 1.34E−122 |
| Muscle/ICCs | Tpm1 | 3.61E−118 |
| Muscle/ICCs | Gm26632 | 1.21E−111 |
| Muscle/ICCs | Tagln | 2.73E−110 |
| Muscle/ICCs | Pdlim3 | 4.40E−108 |
| Muscle/ICCs | Trps1 | 1.44E−107 |
| Muscle/ICCs | Sema3a | 1.91E−107 |
| Muscle/ICCs | Malat1 | 1.59E−103 |
| Muscle/ICCs | Smtn | 7.23E−100 |
| Muscle/ICCs | Myl9 | 6.75E−99 |
| Muscle/ICCs | Dlc1 | 6.91E−99 |
| Muscle/ICCs | Pgm5 | 2.14E−98 |
| Muscle/ICCs | Pcdh7 | 2.18E−95 |
| Muscle/ICCs | Myl6 | 3.36E−93 |
| Muscle/ICCs | Des | 1.19E−92 |
| Muscle/ICCs | Csrp1 | 2.64E−91 |
| Muscle/ICCs | Ppm1e | 4.07E−89 |
| Muscle/ICCs | Enah | 1.67E−88 |
| Muscle/ICCs | Meis1 | 1.13E−86 |
| Muscle/ICCs | Atp2b4 | 2.09E−85 |
| Muscle/ICCs | Itga1 | 3.04E−83 |
| Muscle/ICCs | Cacna2d1 | 3.50E−82 |
| Muscle/ICCs | Adam33 | 2.64E−81 |
| Muscle/ICCs | Hmcn2 | 1.20E−80 |
| Muscle/ICCs | Sparcl1 | 1.64E−80 |
| Muscle/ICCs | Sgcd | 9.25E−80 |
| Muscle/ICCs | Slc8a1 | 1.10E−79 |
| Muscle/ICCs | Sorbs1 | 1.39E−77 |
| Muscle/ICCs | Zfpm2 | 1.97E−77 |
| Muscle/ICCs | Arhgap6 | 2.27E−77 |
| Muscle/ICCs | Pde4b | 4.38E−75 |
| Muscle/ICCs | Cnn1 | 5.11E−75 |
| Muscle/ICCs | Ppp1r12b | 1.69E−74 |
| Muscle/ICCs | Clmp | 2.10E−74 |
| Muscle/ICCs | Myocd | 7.66E−74 |
| Muscle/ICCs | Ryr2 | 9.62E−74 |
| Muscle/ICCs | Pbx3 | 1.37E−73 |
| Muscle/ICCs | Efna5 | 5.17E−73 |
| Muscle/ICCs | Fnbp1 | 3.80E−70 |
| Muscle/ICCs | Msrb3 | 1.13E−69 |
| Muscle/ICCs | Tns1 | 3.87E−69 |
| Muscle/ICCs | Epha7 | 4.02E−69 |
| Muscle/ICCs | Ryr3 | 4.05E−69 |
| Muscle/ICCs | Abcc9 | 1.25E−68 |
| Muscle/ICCs | Kcnma1 | 5.95E−68 |
| Muscle/ICCs | Lmod1 | 1.91E−66 |
| Muscle/ICCs | Nexn | 6.32E−66 |
| Muscle/ICCs | Chrm2 | 1.48E−64 |
| Muscle/ICCs | Ctnna3 | 2.02E−64 |
| Muscle/ICCs | Col5a2 | 3.24E−64 |
| Muscle/ICCs | Prickle1 | 5.16E−64 |
| Muscle/ICCs | Kcnip1 | 6.33E−64 |
| Muscle/ICCs | Bnc2 | 2.55E−63 |
| Muscle/ICCs | Filip1 | 4.30E−63 |
| Muscle/ICCs | Ckb | 1.23E−62 |
| Muscle/ICCs | Akap6 | 1.86E−62 |
| Muscle/ICCs | Dtna | 1.07E−61 |
| Muscle/ICCs | Pdzrn3 | 1.31E−61 |
| Muscle/ICCs | Pdlim7 | 1.89E−61 |
| Muscle/ICCs | Tpm2 | 3.01E−61 |
| Muscle/ICCs | Plcb1 | 8.98E−61 |
| Muscle/ICCs | Rora | 1.01E−59 |
| Muscle/ICCs | Tnc | 1.73E−59 |
| Muscle/ICCs | Ppp1r12a | 2.53E−58 |
| Muscle/ICCs | Tgfbr3 | 5.35E−58 |
| Muscle/ICCs | Samd4 | 1.09E−57 |
| Muscle/ICCs | Antxrl | 1.52E−56 |
| Muscle/ICCs | Eml1 | 1.29E−55 |
| Muscle/ICCs | Nxn | 1.67E−55 |
| Muscle/ICCs | Zbtb20 | 2.83E−55 |
| Muscle/ICCs | Colec12 | 3.79E−55 |
| Muscle/ICCs | Cap2 | 4.50E−53 |
| Muscle/ICCs | Kcnd3 | 9.63E−53 |
| Muscle/ICCs | Fam129a | 6.34E−52 |
| Muscle/ICCs | Pdzrn4 | 9.83E−52 |
| Muscle/ICCs | Mrvi1 | 1.92E−51 |
| Muscle/ICCs | Itga5 | 1.59E−50 |
| Muscle/ICCs | Magi2 | 5.25E−50 |
| Muscle/ICCs | Ltbp1 | 1.05E−48 |
| Muscle/ICCs | Dpp10 | 5.28E−47 |
| Muscle/ICCs | Dmpk | 8.80E−47 |
| Muscle/ICCs | Satb1 | 4.09E−46 |
| Muscle/ICCs | Wscd2 | 8.36E−42 |
| Muscle/ICCs | Ppp1r14a | 4.34E−41 |
| Muscle/ICCs | Cnn2 | 6.67E−40 |
| Muscle/ICCs | Gm10848 | 1.39E−39 |
| Muscle/ICCs | Jph2 | 2.41E−39 |
| Muscle/ICCs | Hspb1 | 1.21E−38 |
| Muscle/ICCs | Pamr1 | 1.50E−34 |
| Muscle/ICCs | Klhl23 | 1.53E−33 |
| Muscle/ICCs | Grem2 | 8.70E−33 |
| Muscle/ICCs | Rspo3 | 3.10E−32 |
| Muscle/ICCs | A630023A22Rik | 4.47E−32 |
| Muscle/ICCs | Slc7a8 | 1.33E−31 |
| Muscle/ICCs | C1qtnf7 | 2.11E−31 |
| Muscle/ICCs | Mapk4 | 2.40E−30 |
| Muscle/ICCs | Flnc | 1.17E−28 |
| Muscle/ICCs | Mcam | 2.81E−28 |
| Muscle/ICCs | Rgs5 | 8.47E−28 |
| Muscle/ICCs | Frem2 | 1.41E−27 |
| Muscle/ICCs | Tspan2 | 5.23E−27 |
| Muscle/ICCs | Fgf10 | 9.11E−27 |
| Muscle/ICCs | Ptn | 6.40E−26 |
| Muscle/ICCs | Cspg4 | 9.45E−26 |
| Muscle/ICCs | Rgs1 | 1.16E−25 |
| Muscle/ICCs | Gem | 1.82E−24 |
| Muscle/ICCs | Fendrr | 4.06E−24 |
| Muscle/ICCs | Atcayos | 1.44E−23 |
| Muscle/ICCs | Mab21l2 | 9.14E−22 |
| Muscle/ICCs | Slc2a4 | 3.00E−21 |
| Muscle/ICCs | Aoc3 | 1.89E−20 |
| Muscle/ICCs | Tgfb1i1 | 2.32E−20 |
| Muscle/ICCs | Gm13269 | 9.26E−20 |
| Muscle/ICCs | Scube2 | 9.64E−20 |
| Muscle/ICCs | Kcnj8 | 1.03E−19 |
| Muscle/ICCs | Slc6a17 | 1.98E−19 |
| Muscle/ICCs | Mettl24 | 1.55E−17 |
| Muscle/ICCs | Kcnb1 | 2.15E−16 |
| Muscle/ICCs | Pln | 4.42E−15 |
| Muscle/ICCs | Ctxn3 | 4.33E−14 |
| Muscle/ICCs | Colec10 | 6.01E−14 |
| Muscle/ICCs | Gm16141 | 1.53E−13 |
| Muscle/ICCs | Kcnmb1 | 4.05E−13 |
| Muscle/ICCs | Cyr61 | 7.09E−13 |
| Muscle/ICCs | Igfbp2 | 1.15E−12 |
| Muscle/ICCs | Olfml2b | 1.20E−12 |
| Muscle/ICCs | Actn2 | 1.65E−12 |
| Muscle/ICCs | Nkd1 | 1.96E−12 |
| Muscle/ICCs | Grem1 | 5.23E−12 |
| Muscle/ICCs | Lrch2 | 5.81E−12 |
| Muscle/ICCs | Adrb3 | 3.88E−11 |
| Muscle/ICCs | Egflam | 1.16E−10 |
| Muscle/ICCs | Trnp1 | 1.92E−09 |
| Muscle/ICCs | Il17d | 3.67E−09 |
| Muscle/ICCs | Cyp7b1 | 4.35E−09 |
| Muscle/ICCs | Shisa4 | 2.84E−08 |
| Muscle/ICCs | Dkk2 | 4.32E−08 |
| Muscle/ICCs | A630012P03Rik | 5.46E−08 |
| Neuron | Syt1 | 1.08E−136 |
| Neuron | Snap25 | 2.16E−136 |
| Neuron | Snhg11 | 7.18E−136 |
| Neuron | Elavl4 | 2.69E−130 |
| Neuron | Ank2 | 2.11E−121 |
| Neuron | Map1b | 1.05E−120 |
| Neuron | Rab3c | 3.69E−120 |
| Neuron | Fgf13 | 3.76E−119 |
| Neuron | Slc7a14 | 5.37E−119 |
| Neuron | Celf4 | 2.13E−117 |
| Neuron | Ncam2 | 2.76E−114 |
| Neuron | Kcnq3 | 5.28E−112 |
| Neuron | Dpp6 | 1.99E−111 |
| Neuron | Cntn1 | 2.21E−110 |
| Neuron | Fam155a | 5.59E−110 |
| Neuron | Pcbp3 | 1.20E−108 |
| Neuron | Mapk10 | 1.26E−107 |
| Neuron | Rtn1 | 1.39E−103 |
| Neuron | Prph | 1.62E−102 |
| Neuron | Nrg3 | 1.62E−102 |
| Neuron | Ctnna2 | 4.56E−102 |
| Neuron | Kcnc2 | 2.12E−101 |
| Neuron | Cadm1 | 9.82E−101 |
| Neuron | Rit2 | 1.68E−100 |
| Neuron | Elavl3 | 3.01E−100 |
| Neuron | Sgip1 | 4.63E−100 |
| Neuron | Khdrbs2 | 2.48E−99 |
| Neuron | Clvs1 | 6.47E−98 |
| Neuron | Ncam1 | 3.88E−97 |
| Neuron | Nrxn1 | 4.30E−97 |
| Neuron | Fgf14 | 1.26E−96 |
| Neuron | Ppfia2 | 5.48E−96 |
| Neuron | Mdga2 | 3.03E−95 |
| Neuron | Stmn2 | 1.09E−94 |
| Neuron | Gabrb3 | 1.61E−94 |
| Neuron | Pcdh15 | 2.92E−94 |
| Neuron | Kcnip4 | 3.64E−94 |
| Neuron | Gria4 | 4.20E−94 |
| Neuron | Lrfn5 | 8.08E−94 |
| Neuron | Kif5a | 9.38E−94 |
| Neuron | Spock2 | 1.05E−93 |
| Neuron | Map2 | 4.06E−93 |
| Neuron | Lrrtm4 | 9.41E−93 |
| Neuron | Gria2 | 2.28E−92 |
| Neuron | Enah | 1.04E−91 |
| Neuron | Nrg3os | 2.03E−91 |
| Neuron | Ptprn | 2.92E−91 |
| Neuron | Pclo | 5.01E−91 |
| Neuron | Cadps | 1.32E−90 |
| Neuron | Nav3 | 3.22E−90 |
| Neuron | Kcnb2 | 1.05E−89 |
| Neuron | Negr1 | 2.19E−89 |
| Neuron | Akap6 | 5.81E−89 |
| Neuron | Garnl3 | 6.56E−89 |
| Neuron | Erc2 | 1.12E−87 |
| Neuron | Kcnt2 | 1.39E−87 |
| Neuron | Unc79 | 1.47E−87 |
| Neuron | Myt1l | 1.14E−86 |
| Neuron | Cdh2 | 2.71E−86 |
| Neuron | Fstl5 | 3.50E−86 |
| Neuron | Grid2 | 5.07E−86 |
| Neuron | Dgki | 1.42E−85 |
| Neuron | Dscam | 3.83E−85 |
| Neuron | Sntg1 | 3.04E−84 |
| Neuron | Mapt | 4.60E−84 |
| Neuron | Nrxn2 | 6.12E−83 |
| Neuron | Eml5 | 2.15E−82 |
| Neuron | Fam163a | 4.82E−82 |
| Neuron | Stmn3 | 6.69E−82 |
| Neuron | Pcsk1n | 1.08E−81 |
| Neuron | Chrna3 | 1.04E−80 |
| Neuron | Snap91 | 1.04E−80 |
| Neuron | Ndst4 | 3.20E−80 |
| Neuron | Scg2 | 3.99E−80 |
| Neuron | Frmd4a | 7.91E−80 |
| Neuron | Reep1 | 8.89E−80 |
| Neuron | Unc80 | 1.01E−79 |
| Neuron | Ctnnd2 | 1.67E−79 |
| Neuron | Hmgn3 | 2.09E−79 |
| Neuron | Syt11 | 3.94E−79 |
| Neuron | Gabrg3 | 6.68E−78 |
| Neuron | Nap1l5 | 1.15E−77 |
| Neuron | Trpm3 | 1.28E−77 |
| Neuron | Ret | 1.35E−77 |
| Neuron | Cacna2d1 | 1.96E−77 |
| Neuron | Pcsk2 | 2.08E−77 |
| Neuron | Dlg2 | 5.68E−77 |
| Neuron | Ppp2r2b | 6.81E−77 |
| Neuron | Fhod3 | 8.09E−77 |
| Neuron | Nsg2 | 8.13E−77 |
| Neuron | Rbms3 | 4.66E−76 |
| Neuron | Ahi1 | 6.16E−76 |
| Neuron | Pirt | 1.28E−75 |
| Neuron | Kcnd2 | 1.42E−75 |
| Neuron | Meg3 | 5.86E−75 |
| Neuron | Lix1 | 6.30E−75 |
| Neuron | Chrm2 | 2.12E−74 |
| Neuron | Prkcb | 2.49E−74 |
| Neuron | Celf3 | 4.29E−74 |
| Neuron | Scn3a | 6.64E−74 |
| Neuron | Ina | 2.47E−60 |
| Neuron | Hs3st2 | 1.45E−56 |
| Neuron | Tmem179 | 8.08E−54 |
| Neuron | Mapk8ip2 | 6.72E−52 |
| Neuron | Doc2b | 3.76E−49 |
| Neuron | Gdap1 | 3.08E−48 |
| Neuron | Gpr22 | 1.62E−45 |
| Neuron | Rims3 | 1.62E−45 |
| Neuron | P2rx2 | 1.27E−44 |
| Neuron | Atp2b3 | 6.29E−42 |
| Neuron | Vgf | 1.64E−37 |
| Neuron | Cend1 | 1.64E−37 |
| Neuron | Golga7b | 9.28E−36 |
| Neuron | Ap3b2 | 9.28E−36 |
| Neuron | Oprk1 | 6.95E−35 |
| Neuron | Clip3 | 6.95E−35 |
| Neuron | Dusp26 | 5.06E−34 |
| Neuron | Tmem59l | 5.06E−34 |
| Neuron | Tpbgl | 5.06E−34 |
| Neuron | Gm11418 | 2.69E−32 |
| Neuron | Slc10a4 | 2.69E−32 |
| Neuron | Abcg4 | 1.99E−31 |
| Neuron | Hpcal4 | 1.44E−30 |
| Neuron | Pnmal1 | 1.44E−30 |
| Neuron | Kcnc1 | 1.44E−30 |
| Neuron | Elovl4 | 1.04E−29 |
| Neuron | Ly6h | 7.28E−29 |
| Neuron | Slc35d3 | 5.17E−28 |
| Neuron | Celsr3 | 5.17E−28 |
| Neuron | Lhfpl4 | 3.63E−27 |
| Neuron | Schip1 | 3.63E−27 |
| Neuron | Rprml | 2.54E−26 |
| Neuron | Cnih2 | 2.54E−26 |
| Neuron | Cartpt | 1.76E−25 |
| Neuron | Htr3a | 1.76E−25 |
| Neuron | Arhgdig | 1.76E−25 |
| Neuron | Necab2 | 1.76E−25 |
| Neuron | Rnf112 | 1.76E−25 |
| Neuron | Sprn | 1.21E−24 |
| Neuron | Sncb | 5.64E−23 |
| Neuron | Tro | 5.64E−23 |
| Neuron | Sycp2 | 5.64E−23 |
| Neuron | Elmod1 | 3.85E−22 |
| Neuron | Cdk5r2 | 3.85E−22 |
| Neuron | Gm38112 | 2.60E−21 |
| Neuron | Gm10419 | 2.60E−21 |
| Neuron | Elavl2 | 1.75E−20 |
| Neuron | Shc2 | 1.75E−20 |
| Neuron | Tmc3 | 1.17E−19 |
| Neuron | Lhfpl5 | 1.17E−19 |
| Neuron | Rab9b | 7.67E−19 |
| Neuron | Frrs1l | 7.67E−19 |
| Neuron | Brsk2 | 7.67E−19 |
| Neuron | Bglap | 5.10E−18 |
| Neuron | Rltpr | 5.10E−18 |
| Neuron | Mir124a-1hg | 5.10E−18 |
| Neuron | Rtn2 | 3.34E−17 |
| Neuron | Gpr27 | 3.34E−17 |
| Neuron | Srsf12 | 3.34E−17 |
| Neuron | Oprl1 | 3.34E−17 |
| Neuron | Tmem121 | 3.34E−17 |
| Neuron | Nefh | 3.34E−17 |
| Neuron | Ttc9b | 2.18E−16 |
| Neuron | Kcnc4 | 2.18E−16 |
| Neuron | Pcsk2os1 | 2.18E−16 |
| Neuron | Fam131b | 2.18E−16 |
| Neuron | Sox11 | 1.43E−15 |
| Neuron | Tram1l1 | 1.43E−15 |
| Neuron | Ankrd45 | 1.43E−15 |
| Neuron | Calcb | 9.33E−15 |
| Neuron | Dbh | 9.33E−15 |
| Neuron | Fbxw15 | 9.33E−15 |
| Neuron | Mfap2 | 9.33E−15 |
| Neuron | Epha8 | 9.33E−15 |
| Neuron | Inha | 9.33E−15 |
| Neuron | Rasl10b | 6.02E−14 |
| Neuron | Adcy1 | 6.02E−14 |
| Neuron | Slitrk5 | 6.02E−14 |
| Neuron | RP23-291B1.2 | 3.87E−13 |
| Neuron | Fibcd1 | 3.87E−13 |
| Neuron | Diras1 | 3.87E−13 |
| Neuron | Gm10605 | 2.50E−12 |
| Neuron | Ephb6 | 2.50E−12 |
| Neuron | Prokr1 | 2.50E−12 |
| Neuron | Gpr61 | 2.50E−12 |
| Neuron | Tmem145 | 2.50E−12 |
| Neuron | Oxtr | 1.60E−11 |
| Neuron | Nudt11 | 1.60E−11 |
| Neuron | Kcnv1 | 1.60E−11 |
| Neuron | Grp | 1.02E−10 |
| Neuron | Islr2 | 1.02E−10 |
| Neuron | Gm11342 | 1.02E−10 |
| Neuron | Gm37640 | 1.02E−10 |
| Neuron | Cngb1 | 1.02E−10 |
| Neuron | Zkscan2 | 1.02E−10 |
| Neuron | Kcnj5 | 6.51E−10 |
| Neuron | Cbln2 | 4.08E−09 |
| Neuron | Slc17a6 | 4.08E−09 |
| Neuron | Zik1 | 4.08E−09 |
| Neuron | Gm12130 | 4.08E−09 |
| T_cells | Mir142hg | 1.00E−153 |
| T_cells | Arhgap15 | 5.67E−127 |
| T_cells | Hmha1 | 9.54E−116 |
| T_cells | Ebf1 | 2.66E−93 |
| T_cells | Ptprc | 5.64E−88 |
| T_cells | Gm26740 | 3.29E−86 |
| T_cells | Bank1 | 5.56E−82 |
| T_cells | Gm43291 | 2.71E−81 |
| T_cells | Ikzf3 | 5.21E−72 |
| T_cells | Gimap6 | 5.89E−69 |
| T_cells | Dock2 | 1.71E−66 |
| T_cells | Dock10 | 6.41E−63 |
| T_cells | Gm43603 | 2.18E−60 |
| T_cells | Cd79a | 1.87E−59 |
| T_cells | Cd74 | 5.75E−59 |
| T_cells | Mef2c | 4.97E−56 |
| T_cells | Mbnl1 | 1.10E−54 |
| T_cells | Fam65b | 6.08E−49 |
| T_cells | Bach2 | 1.89E−46 |
| T_cells | Man1a | 1.89E−46 |
| T_cells | Ccnd3 | 3.02E−45 |
| T_cells | Ly6e | 6.59E−45 |
| T_cells | Lcp1 | 1.18E−44 |
| T_cells | Ikzf1 | 3.77E−43 |
| T_cells | Aff3 | 7.77E−41 |
| T_cells | Rhoh | 2.46E−40 |
| T_cells | Inpp5d | 7.10E−40 |
| T_cells | Gimap4 | 7.00E−36 |
| T_cells | Bcl2 | 1.24E−35 |
| T_cells | Siglecg | 1.12E−34 |
| T_cells | Fli1 | 3.19E−34 |
| T_cells | Kcnq5 | 3.41E−34 |
| T_cells | Prkcb | 7.51E−34 |
| T_cells | Tspan32 | 1.09E−31 |
| T_cells | Gm43388 | 5.33E−31 |
| T_cells | St6galnac3 | 1.33E−30 |
| T_cells | Apobec3 | 3.70E−30 |
| T_cells | Cd79b | 7.49E−30 |
| T_cells | Itga4 | 2.62E−29 |
| T_cells | Pax5 | 4.57E−29 |
| T_cells | Cd52 | 3.98E−28 |
| T_cells | H2-D1 | 5.82E−28 |
| T_cells | Ptpn22 | 6.23E−28 |
| T_cells | Ppp1r16b | 8.44E−28 |
| T_cells | Coro1a | 9.27E−28 |
| T_cells | Rabgap1l | 6.37E−27 |
| T_cells | Tbc1d10c | 1.74E−26 |
| T_cells | Foxp1 | 1.91E−26 |
| T_cells | Stat4 | 1.95E−26 |
| T_cells | Ralgps2 | 2.95E−26 |
| T_cells | Ptprcap | 1.57E−25 |
| T_cells | Skap1 | 1.62E−25 |
| T_cells | H2-Aa | 1.68E−25 |
| T_cells | Srgn | 2.23E−24 |
| T_cells | Gm17660 | 2.50E−24 |
| T_cells | Lyn | 4.88E−24 |
| T_cells | Arhgap30 | 6.09E−24 |
| T_cells | H2-Eb1 | 8.16E−24 |
| T_cells | H2-Ab1 | 8.76E−24 |
| T_cells | Laptm5 | 8.97E−24 |
| T_cells | Tespa1 | 1.15E−23 |
| T_cells | Inpp4b | 1.15E−23 |
| T_cells | Gm15987 | 2.11E−23 |
| T_cells | Pou2f2 | 4.61E−23 |
| T_cells | Sh3kbp1 | 5.66E−23 |
| T_cells | Blk | 7.22E−23 |
| T_cells | Mndal | 7.25E−23 |
| T_cells | Wdfy4 | 2.19E−22 |
| T_cells | Gm10552 | 2.64E−22 |
| T_cells | Rbm39 | 2.89E−22 |
| T_cells | Gm20388 | 3.70E−22 |
| T_cells | Shisa5 | 9.19E−22 |
| T_cells | Fermt3 | 1.47E−21 |
| T_cells | Nedd9 | 1.87E−21 |
| T_cells | Tmem163 | 2.04E−21 |
| T_cells | Dock11 | 4.87E−21 |
| T_cells | Ets1 | 1.71E−20 |
| T_cells | AU020206 | 2.91E−20 |
| T_cells | Cd53 | 2.93E−20 |
| T_cells | Ly86 | 3.67E−20 |
| T_cells | Cd84 | 3.78E−20 |
| T_cells | Serinc3 | 4.16E−20 |
| T_cells | Btla | 4.27E−20 |
| T_cells | Sp100 | 4.42E−20 |
| T_cells | Jchain | 4.73E−20 |
| T_cells | Acap1 | 1.55E−19 |
| T_cells | Fchsd2 | 1.61E−19 |
| T_cells | Slamf6 | 1.73E−19 |
| T_cells | Rasgrp3 | 2.21E−19 |
| T_cells | Lax1 | 2.60E−19 |
| T_cells | Ankrd44 | 2.94E−19 |
| T_cells | Fam49b | 3.59E−19 |
| T_cells | C130026I21Rik | 4.39E−19 |
| T_cells | Hivep2 | 6.48E−19 |
| T_cells | Ly6d | 6.60E−19 |
| T_cells | Ltb | 7.40E−19 |
| T_cells | Cd48 | 7.47E−19 |
| T_cells | Pik3cd | 1.09E−18 |
| T_cells | Cr2 | 1.40E−18 |
| T_cells | Raet1e | 3.46E−18 |
| T_cells | Tnfrsf13b | 5.84E−18 |
| T_cells | Fcrl1 | 7.23E−18 |
| T_cells | Cd37 | 7.92E−18 |
| T_cells | Gpr132 | 6.84E−17 |
| T_cells | Klrd1 | 8.09E−17 |
| T_cells | Tnfrsf13c | 3.07E−16 |
| T_cells | Pou2af1 | 4.16E−16 |
| T_cells | Dok3 | 8.98E−16 |
| T_cells | Stap1 | 1.16E−15 |
| T_cells | H2-DMb2 | 4.30E−15 |
| T_cells | Rac2 | 4.84E−15 |
| T_cells | Nrros | 1.21E−14 |
| T_cells | Cd72 | 2.02E−14 |
| T_cells | Gimap3 | 5.38E−14 |
| T_cells | Napsa | 7.25E−14 |
| T_cells | Clec2i | 1.06E−13 |
| T_cells | Selplg | 1.72E−13 |
| T_cells | Rasgrp1 | 1.97E−13 |
| T_cells | Dusp2 | 2.54E−13 |
| T_cells | Nxpe3 | 2.68E−13 |
| T_cells | Rinl | 6.25E−13 |
| T_cells | Nckap1l | 7.23E−13 |
| T_cells | Cyfip2 | 1.43E−12 |
| T_cells | Lck | 1.93E−12 |
| T_cells | AI662270 | 2.23E−12 |
| T_cells | Ctsw | 2.55E−12 |
| T_cells | Ciita | 4.99E−12 |
| T_cells | Mirt1 | 1.11E−11 |
| T_cells | Clnk | 1.13E−11 |
| T_cells | H2-Ob | 1.27E−11 |
| T_cells | Sp140 | 1.99E−11 |
| T_cells | Cd22 | 2.38E−11 |
| T_cells | Fcer2a | 3.12E−11 |
| T_cells | Ccr6 | 3.12E−11 |
| T_cells | Gm16152 | 9.69E−11 |
| T_cells | Myo1g | 2.53E−10 |
| T_cells | Mzb1 | 3.81E−10 |
| T_cells | Runx3 | 9.65E−10 |
| T_cells | Gm16158 | 1.17E−09 |
| T_cells | H2-Q6 | 1.60E−09 |
| T_cells | Arhgdib | 2.20E−09 |
| T_cells | Grap2 | 2.89E−09 |
| T_cells | Derl3 | 3.82E−09 |
| T_cells | Txk | 9.35E−09 |
| T_cells | Adrb2 | 1.73E−08 |
| T_cells | Cd226 | 2.27E−08 |
| T_cells | Ccl5 | 2.28E−08 |
| T_cells | 5031414D18Rik | 2.93E−08 |
| T_cells | H2-DMb1 | 3.38E−08 |
| T_cells | Gm28053 | 8.83E−08 |
| T_cells | Cd244 | 2.23E−07 |
| Tuft | St18 | 4.65E−124 |
| Tuft | Dclk1 | 1.03E−116 |
| Tuft | Sh2d6 | 4.94E−107 |
| Tuft | Rgs13 | 5.84E−93 |
| Tuft | Nebl | 1.82E−89 |
| Tuft | Fyb | 2.92E−70 |
| Tuft | Dgki | 1.02E−68 |
| Tuft | Gnat3 | 2.02E−59 |
| Tuft | Ccdc129 | 6.11E−56 |
| Tuft | Pik3r5 | 3.24E−53 |
| Tuft | Hck | 4.27E−49 |
| Tuft | Avil | 9.92E−49 |
| Tuft | Pstpip2 | 1.72E−48 |
| Tuft | Plcg2 | 8.72E−47 |
| Tuft | Dnah5 | 5.15E−46 |
| Tuft | Matk | 1.32E−44 |
| Tuft | Lrmp | 1.83E−44 |
| Tuft | Chat | 2.41E−43 |
| Tuft | Inpp5d | 6.69E−42 |
| Tuft | Mast4 | 5.86E−39 |
| Tuft | Strip2 | 2.47E−38 |
| Tuft | Pde4d | 4.39E−37 |
| Tuft | Slc9a9 | 1.57E−36 |
| Tuft | Runx1 | 3.73E−36 |
| Tuft | Pou2f3 | 8.43E−36 |
| Tuft | Adh1 | 1.40E−34 |
| Tuft | Bmx | 2.19E−34 |
| Tuft | Trpm5 | 8.90E−33 |
| Tuft | Chn2 | 3.90E−32 |
| Tuft | Ltc4s | 9.35E−32 |
| Tuft | Pik3cg | 2.69E−30 |
| Tuft | Ppp3ca | 5.73E−30 |
| Tuft | Nav2 | 9.66E−30 |
| Tuft | Sh2d7 | 1.27E−29 |
| Tuft | Ptpn18 | 1.75E−29 |
| Tuft | Cd24a | 3.20E−29 |
| Tuft | Gm21954 | 3.67E−28 |
| Tuft | Il13ra1 | 8.86E−27 |
| Tuft | Malrd1 | 1.25E−26 |
| Tuft | Map2 | 4.59E−26 |
| Tuft | Alox5 | 6.04E−26 |
| Tuft | Myo1b | 2.70E−25 |
| Tuft | Fam19a1 | 1.42E−23 |
| Tuft | 1810046K07Rik | 1.49E−23 |
| Tuft | Ano6 | 6.00E−23 |
| Tuft | Ptprc | 2.26E−22 |
| Tuft | Hpgds | 1.33E−21 |
| Tuft | Cd300lf | 1.79E−21 |
| Tuft | Spib | 2.37E−21 |
| Tuft | Abhd18 | 2.95E−21 |
| Tuft | Pygl | 1.35E−19 |
| Tuft | A630010A05Rik | 3.64E−19 |
| Tuft | Lima1 | 4.43E−19 |
| Tuft | Adgrb3 | 2.24E−18 |
| Tuft | F930017D23Rik | 2.41E−18 |
| Tuft | Suco | 2.41E−18 |
| Tuft | Bub3 | 3.45E−18 |
| Tuft | Rgs22 | 5.61E−18 |
| Tuft | Vav1 | 7.02E−18 |
| Tuft | Fam221a | 7.23E−18 |
| Tuft | Abhd2 | 7.46E−18 |
| Tuft | Rabgap1l | 2.76E−17 |
| Tuft | Tmem116 | 4.38E−17 |
| Tuft | Hmx2 | 5.73E−17 |
| Tuft | Lyn | 7.67E−17 |
| Tuft | Ptprj | 8.08E−17 |
| Tuft | Gm609 | 4.06E−16 |
| Tuft | 1700112E06Rik | 5.87E−16 |
| Tuft | Siglecf | 6.81E−15 |
| Tuft | Ppp1r14c | 7.34E−15 |
| Tuft | Pnpla3 | 7.57E−15 |
| Tuft | BCl2l14 | 7.57E−15 |
| Tuft | Ly6g6d | 1.32E−14 |
| Tuft | Mlip | 1.75E−14 |
| Tuft | Man2a1 | 1.79E−14 |
| Tuft | Adcy2 | 2.47E−14 |
| Tuft | Fryl | 3.24E−14 |
| Tuft | Zfhx3 | 3.02E−13 |
| Tuft | Larp1b | 4.15E−13 |
| Tuft | Tmem245 | 6.70E−13 |
| Tuft | Zbtb20 | 7.97E−13 |
| Tuft | Hyal5 | 9.54E−13 |
| Tuft | Ptgs1 | 1.09E−12 |
| Tuft | Tanc2 | 1.23E−12 |
| Tuft | Cacnb4 | 1.61E−12 |
| Tuft | Txndc16 | 1.65E−12 |
| Tuft | Oxr1 | 1.67E−12 |
| Tuft | Itpr2 | 2.54E−12 |
| Tuft | 1700111E14Rik | 2.62E−12 |
| Tuft | Il17rb | 5.79E−12 |
| Tuft | Gnai1 | 6.11E−12 |
| Tuft | Gpc6 | 1.34E−11 |
| Tuft | Zdhhc17 | 3.27E−11 |
| Tuft | Ahnak2 | 5.11E−11 |
| Tuft | Man1a | 5.46E−11 |
| Tuft | Gm2245 | 8.26E−11 |
| Tuft | Rac2 | 9.05E−11 |
| Tuft | Espn | 1.30E−10 |
| Tuft | Tspan6 | 1.42E−10 |
| Tuft | Tnik | 1.50E−10 |
| Tuft | Kctd12 | 2.27E−10 |
| Tuft | Cdkn1c | 2.28E−10 |
| Tuft | Dmxl2 | 2.71E−10 |
| Tuft | Ccnj | 2.87E−10 |
| Tuft | Snrnp25 | 4.10E−10 |
| Tuft | Slco4a1 | 2.61E−09 |
| Tuft | Gm42609 | 1.35E−08 |
| Tuft | Ttll11 | 2.81E−08 |
| Tuft | Cyp2j13 | 3.58E−08 |
| Tuft | Ly6g6f | 1.42E−07 |
| Tuft | Trim38 | 2.20E−07 |
| Tuft | Pea15a | 7.26E−07 |
| Tuft | Plcb2 | 1.40E−06 |
| Tuft | Crisp3 | 2.60E−06 |
| Tuft | Agt | 3.32E−06 |
| Tuft | Adcy5 | 1.11E−05 |
| Tuft | Hap1 | 1.52E−05 |
| Tuft | Kcnq4 | 1.86E−05 |
| Tuft | Msi1 | 2.13E−05 |
| Tuft | Gfi1b | 2.81E−05 |
| Tuft | Ffar3 | 2.81E−05 |
| Tuft | Sptb | 5.02E−05 |
| Tuft | Gm4952 | 5.69E−05 |
| Tuft | Adam22 | 1.05E−04 |
| Tuft | Limd2 | 1.54E−04 |
| Tuft | Nrg4 | 4.72E−04 |
| Tuft | Srpx2 | 9.96E−04 |
| Tuft | Alox5ap | 1.22E−03 |
| Tuft | Fcnaos | 1.83E−03 |
| Tuft | Ankrd33b | 2.02E−03 |
| Tuft | Grin2b | 5.03E−03 |
| Tuft | Gm2447 | 1.33E−02 |
| Tuft | Krt23 | 1.56E−02 |
| Tuft | Kcnk3 | 1.56E−02 |
| Tuft | Cabp1 | 2.62E−02 |
| Tuft | Drd3 | 2.64E−02 |
| Tuft | Dlgap3 | 3.16E−02 |
| Tuft | Lzts3 | 4.16E−02 |
| Tuft | Pkp1 | 4.35E−02 |

TABLE 18
Genes comprising signatures in FIG. 21B. Gene(s) encoding either
synthetic enzymes or respective receptors displayed in FIG. 21B.

| Name | Abbreviation | Type | Genes |
|---|---|---|---|
| Acetylcholine | Ach | Synthesis | Chat |
| Nitric oxide | NO | Synthesis | Nos1 |
| Norepinephrine | Norepinephrine | Synthesis | Dbh |
| Calcb | Calcb | Synthesis | Calcb |
| Cartpt | Cartpt | Synthesis | Cartpt |
| Cholecystokinin | Cck | Synthesis | Cck |
| Dynorphin | Dynorphin | Synthesis | Pdyn |
| Enkephalin | Enkephalin | Synthesis | Penk |
| Galanin | Galanin | Synthesis | Gal |
| Gastrin releasing peptide | Grp | Synthesis | Grp |
| Neuromedin U | Nmu | Synthesis | Nmu |
| PACAP | PACAP | Synthesis | Adcyap1 |
| Somatostatin | Sst | Synthesis | Sst |
| Tachykinin | Tachykinin | Synthesis | Tac1 |
| Vasoactive intestinal | Vip | Synthesis | Vip |
| peptide | | | |
| Dynorphin | Dynorphin | Receptor | Oprd1, Oprk1, |
| | | | Oprm1 |
| Enkephalin | Enkephalin | Receptor | Oprd1, Oprm1 |
| Galanin | Galanin | Receptor | Galr1, Galr2 |
| Glucagon | Glucagon | Receptor | Gcgr |
| Glucagon-like peptide-1 | Glp1 | Receptor | Glp1r |
| Glucagon-like peptide-2 | Glp2 | Receptor | Glp2r |
| Neuromedin U | Nmu | Receptor | Nmur1, Nmur2 |
| Oxytocin | Oxytocin | Receptor | Oxtr |
| Secretin | Secretin | Receptor | Sctr |
| Tachykinin | Tachykinin | Receptor | Tacr1 |
| Vasoactive intestinal | Vip | Receptor | Vipr1, Vipr2, |
| peptide | | | Adcyap1r1 |

Tables 19-22. Summary and marker genes for human ENS atlas. (Table 19) Description of each patient and sample profiled in this study, including age, sex, and colon location. Marker genes for all (Table 20) cells, (Table 21) neurons, and (Table 22) glia from the human muscularis propria profiled with droplet-based 10× sequencing.

TABLE 19

| Patient_ID | Sample_ID | Location_ID | Age | Gender |
|---|---|---|---|---|
| PID_405 | ColFr0_Mye_1 | N/A | 78 | F |
| PID_405 | ColFr0_Muc_2 | N/A | 78 | F |
| PID_405 | ColFr0_Mye_3 | N/A | 78 | F |
| PID_405 | ColFr0_Mye_4 | N/A | 78 | F |
| PID_405 | ColFr0_Mye_5 | N/A | 78 | F |
| PID_405 | ColFr0_Mye_6 | N/A | 78 | F |
| PID_405 | ColFr0_Mye_3b | N/A | 78 | F |
| PID_405 | ColFr0_Mye_4b | N/A | 78 | F |
| PID_405 | ColFr0_Mye_5b | N/A | 78 | F |
| PID_405 | ColFr0_Mye_6b | N/A | 78 | F |
| PID_413 | ColFr0_Sub_1a | Right | 59 | F |
| PID_413 | ColFr0_Sub_1b | Right | 59 | F |
| PID_405 | ColFr0_Mye_7a1 | N/A | 78 | F |
| PID_405 | ColFr0_Mye_7a2 | N/A | 78 | F |
| PID_405 | ColFr0_Mye_7a3 | N/A | 78 | F |
| PID_405 | ColFr0_Mye_7a4 | N/A | 78 | F |
| PID_405 | ColFr0_Mye_7bl | N/A | 78 | F |
| PID_405 | ColFr0_Mye_7b2 | N/A | 78 | F |
| PID_405 | ColFr0_Mye_7b3 | N/A | 78 | F |
| PID_405 | ColFr0_Mye_7b4 | N/A | 78 | F |
| PID_03403 | ColFr0_Mye_8a1 | N/A | 53 | M |
| PID_03403 | ColFr0_Mye_8a2 | N/A | 53 | M |
| PID_03412 | ColFr0_Mye_9 | Left | 42 | M |
| PID_03412 | ColFr0_Sub_2 | Left | 42 | M |
| PID_03412 | ColFr0_Mye_10a1 | Left | 42 | M |
| PID_03412 | ColFr0_Mye_10b1 | Left | 42 | M |
| PID_03412 | ColFr0_Sub_3a1 | Left | 42 | M |
| PID_03412 | ColFr0_Sub_3b1 | Left | 42 | M |
| PID_03445 | ColFr0_Mye_11 | Right | 90 | M |
| PID_03445 | ColFr0_Sub_4a1 | Right | 90 | M |
| PID_03445 | ColFr0_Sub_4a2 | Right | 90 | M |
| PID_03452 | ColFr0_Mye_12 | Right | 56 | F |
| PID_03452 | ColFr0_Sub_5 | Right | 56 | F |
| PID_03403 | ColFr0_Mye_13a1 | N/A | 53 | M |
| PID_03403 | ColFr0_Mye_13a2 | N/A | 53 | M |
| PID_03403 | ColFr0_Mye_13b1 | N/A | 53 | M |
| PID_03403 | ColFr0_Mye_13b2 | N/A | 53 | M |
| PID_03412 | ColFr0_Mye_14a1 | Left | 42 | M |
| PID_03412 | ColFr0_Mye_14a2 | Left | 42 | M |
| PID_03409 | ColFr0_Mye_15a1 | Right | 70 | F |
| PID_03409 | ColFr0_Mye_15a2 | Right | 70 | F |
| PID_432 | ColFr0_Mye_16a1 | Cecum | 72 | F |
| PID_432 | ColFr0_Mye_16a2 | Cecum | 72 | F |
| PID_444 | ColFr0_Mye_17a1 | Right | 60 | M |
| PID_444 | ColFr0_Mye_17a2 | Right | 60 | M |
| PID_03494 | ColFr0_Mye_18a1 | Sigmoid | 35 | M |
| PID_03494 | ColFr0_Mye_18a2 | Sigmoid | 35 | M |
| PID_03494 | ColFr0_Mye_18a3 | Sigmoid | 35 | M |
| PID_03494 | ColFr0_Mye_18a4 | Sigmoid | 35 | M |

TABLE 20

| ident | gene | padjH |
|---|---|---|
| ASMT+ | PPP1R12B | 4.75E−108 |
| ASMT+ | CACNA1C | 3.26E−93 |
| ASMT+ | DMD | 1.20E−85 |
| ASMT+ | PRUNE2 | 6.98E−82 |
| ASMT+ | CALD1 | 2.78E−79 |
| ASMT+ | NEAT1 | 2.78E−79 |
| ASMT+ | ATRNL1 | 3.14E−77 |
| ASMT+ | SORBS1 | 8.81E−76 |
| ASMT+ | PRKG1 | 3.11E−68 |
| ASMT+ | KCNMA1 | 1.66E−67 |
| ASMT+ | LPP | 1.12E−66 |
| ASMT+ | MYH11 | 1.07E−63 |
| ASMT+ | MEIS1 | 1.28E−61 |
| ASMT+ | SYNPO2 | 5.60E−60 |
| ASMT+ | RYR2 | 6.74E−55 |
| ASMT+ | LINC00578 | 5.53E−52 |
| ASMT+ | TPM1 | 1.26E−50 |
| ASMT+ | PCDH7 | 1.33E−49 |
| ASMT+ | ACTN1 | 9.99E−49 |
| ASMT+ | RBPMS | 6.48E−48 |
| ASMT+ | BNC2 | 8.00E−46 |
| ASMT+ | COL6A2 | 3.27E−45 |
| ASMT+ | FOXP2 | 5.30E−45 |
| ASMT+ | FBXO32 | 2.03E−44 |
| ASMT+ | CACNA2D1 | 2.04E−42 |
| ASMT+ | COL4A2 | 6.07E−42 |
| ASMT+ | PDE4D | 6.11E−42 |
| ASMT+ | SULF1 | 1.16E−40 |
| ASMT+ | PDZRN4 | 1.51E−40 |
| ASMT+ | MIR143HG | 1.21E−38 |
| ASMT+ | TNC | 3.35E−37 |
| ASMT+ | ITGA1 | 1.21E−35 |
| ASMT+ | ATP2B4 | 4.58E−34 |
| ASMT+ | RP11-123O10.4 | 1.05E−32 |
| ASMT+ | MYLK | 2.78E−32 |
| ASMT+ | TMTC2 | 3.74E−32 |
| ASMT+ | MEIS2 | 6.93E−32 |
| ASMT+ | SLC8A1 | 1.08E−31 |
| ASMT+ | ADAMTS9-AS2 | 1.89E−31 |
| ASMT+ | COL4A1 | 2.60E−30 |
| ASMT+ | PDE3A | 1.72E−29 |
| ASMT+ | PARD3 | 1.84E−29 |
| ASMT+ | CHRM3 | 1.22E−28 |
| ASMT+ | FN1 | 3.60E−28 |
| ASMT+ | MID1 | 4.18E−28 |
| ASMT+ | DIP2C | 7.46E−27 |
| ASMT+ | PALLD | 1.61E−26 |
| ASMT+ | PDLIM5 | 2.27E−26 |
| ASMT+ | CACNB2 | 3.50E−26 |
| ASMT+ | MIR145 | 6.20E−26 |
| ASMT+ | CDK8 | 6.61E−26 |
| ASMT+ | TTTY14 | 1.18E−25 |
| ASMT+ | TRIO | 1.82E−25 |
| ASMT+ | CHRM2 | 1.99E−25 |
| ASMT+ | AC007392.3 | 4.46E−25 |
| ASMT+ | GPM6A | 6.77E−25 |
| ASMT+ | LMOD1 | 8.21E−25 |
| ASMT+ | SOBP | 4.30E−24 |
| ASMT+ | WLS | 7.67E−24 |
| ASMT+ | TRPS1 | 1.49E−23 |
| ASMT+ | HDAC4 | 1.74E−23 |
| ASMT+ | PDLIM3 | 1.77E−23 |
| ASMT+ | COL6A1 | 1.07E−22 |
| ASMT+ | SMTN | 1.07E−22 |
| ASMT+ | PDZRN3 | 7.13E−22 |
| ASMT+ | LRBA | 1.54E−21 |
| ASMT+ | FENDRR | 1.61E−21 |
| ASMT+ | PRKD1 | 2.70E−21 |
| ASMT+ | PGM5 | 3.03E−21 |
| ASMT+ | SPEG | 4.72E−21 |
| ASMT+ | NBEA | 4.87E−21 |
| ASMT+ | SLFNL1 | 1.16E−20 |
| ASMT+ | LRIG1 | 1.34E−20 |
| ASMT+ | CNTNAP3B | 1.56E−20 |
| ASMT+ | EPHA7 | 3.50E−20 |
| ASMT+ | NAV2 | 1.05E−19 |
| ASMT+ | ARHGEF3 | 4.04E−19 |
| ASMT+ | ZNF248 | 4.72E−19 |
| ASMT+ | ASMT | 9.68E−19 |
| ASMT+ | COL15A1 | 1.32E−18 |
| ASMT+ | SYNM | 1.74E−18 |
| ASMT+ | ABCC9 | 2.94E−18 |
| ASMT+ | MX1 | 4.82E−18 |
| ASMT+ | AP001347.6 | 6.13E−18 |
| ASMT+ | LTBP1 | 7.38E−18 |
| ASMT+ | AKAP6 | 9.01E−18 |
| ASMT+ | CPXM2 | 1.17E−17 |
| ASMT+ | MBD5 | 1.76E−17 |
| ASMT+ | AC098617.1 | 2.50E−17 |
| ASMT+ | BOC | 2.57E−17 |
| ASMT+ | CNTNAP3 | 4.39E−17 |
| ASMT+ | TRPC4 | 6.60E−17 |
| ASMT+ | MAGI2 | 7.48E−17 |
| ASMT+ | GRIP1 | 8.82E−17 |
| ASMT+ | MACF1 | 1.25E−16 |
| ASMT+ | GJC1 | 2.00E−16 |
| ASMT+ | PBX3 | 2.06E−16 |
| ASMT+ | ROR2 | 2.15E−16 |
| ASMT+ | PHF21A | 2.78E−16 |
| ASMT+ | ADAMTSL3 | 2.78E−16 |
| ASMT+ | ST6GALNAC5 | 5.32E−16 |
| ASMT+ | SOGA2 | 7.50E−16 |
| ASMT+ | IFI6 | 4.34E−15 |
| ASMT+ | EMILIN1 | 4.83E−15 |
| ASMT+ | JPH2 | 1.29E−14 |
| ASMT+ | NLGN4Y | 1.08E−13 |
| ASMT+ | PLOD2 | 1.83E−13 |
| ASMT+ | GNAO1 | 2.12E−13 |
| ASMT+ | HEPH | 3.50E−13 |
| ASMT+ | RAB23 | 3.50E−13 |
| ASMT+ | PPP4R1L | 5.05E−13 |
| ASMT+ | STAT1 | 1.10E−12 |
| ASMT+ | IFI44 | 1.55E−12 |
| ASMT+ | HERC6 | 2.08E−12 |
| ASMT+ | RSAD2 | 2.95E−12 |
| ASMT+ | CTC-228N24.2 | 3.30E−12 |
| ASMT+ | CDH11 | 8.47E−12 |
| ASMT+ | SPATS2L | 1.09E−11 |
| ASMT+ | NCS1 | 1.26E−11 |
| ASMT+ | EPSTI1 | 1.65E−11 |
| ASMT+ | AC011043.1 | 2.06E−11 |
| ASMT+ | ADCY5 | 2.17E−11 |
| ASMT+ | ZFHX4 | 4.06E−11 |
| ASMT+ | KLHL23 | 6.02E−11 |
| ASMT+ | HOXD10 | 8.78E−11 |
| ASMT+ | KCND3 | 2.54E−10 |
| ASMT+ | RP11-834C11.3 | 2.83E−10 |
| ASMT+ | ISG15 | 3.76E−10 |
| ASMT+ | NPTN | 6.45E−10 |
| ASMT+ | GPR125 | 1.04E−09 |
| ASMT+ | FAXC | 1.39E−09 |
| ASMT+ | PHKG1 | 7.06E−09 |
| ASMT+ | WNT9A | 1.37E−08 |
| ASMT+ | LINC00278 | 2.84E−08 |
| ASMT+ | NFATC4 | 8.02E−08 |
| ASMT+ | KLHL42 | 8.43E−08 |
| ASMT+ | SLC13A5 | 9.31E−08 |
| ASMT+ | AOC3 | 1.16E−07 |
| ASMT+ | CTD-2127H9.1 | 1.18E−07 |
| ASMT+ | RP11-81N13.1 | 2.37E−07 |
| ASMT+ | PTP4A3 | 2.48E−07 |
| ASMT+ | SYDE2 | 5.84E−07 |
| ASMT+ | AC078941.1 | 7.03E−07 |
| ASMT+ | ARHGEF17 | 7.26E−07 |
| ASMT+ | KDM5D | 1.12E−06 |
| ASMT+ | BTC | 2.19E−06 |
| ASMT+ | KDM3A | 2.64E−06 |
| ASMT+ | SNAP25 | 2.92E−06 |
| ASMT+ | RP11-368L12.1 | 6.27E−06 |
| ASMT+ | EBF4 | 1.48E−05 |
| ASMT+ | IDS | 1.67E−05 |
| ASMT+ | MASP1 | 2.02E−05 |
| ASMT+ | GMPR | 4.48E−05 |
| ASMT+ | IFIT1 | 4.64E−05 |
| ASMT+ | PHLDA3 | 7.92E−05 |
| ASMT+ | MCM8 | 8.66E−05 |
| ASMT+ | CPEB2 | 1.21E−04 |
| ASMT+ | RP4-669L17.10 | 1.71E−04 |
| ASMT+ | SDC3 | 1.83E−04 |
| ASMT+ | FNDC1 | 1.88E−04 |
| ASMT+ | ZBTB1 | 2.07E−04 |
| ASMT+ | COL27A1 | 2.90E−04 |
| ASMT+ | OAS1 | 4.18E−04 |
| ASMT+ | CISD1 | 5.79E−04 |
| ASMT+ | RP11-497G19.1 | 7.32E−04 |
| ASMT+ | HOXD3 | 8.48E−04 |
| ASMT+ | TTTY15 | 1.06E−03 |
| ASMT+ | AC004053.1 | 1.10E−03 |
| ASMT+ | MEMO1 | 1.31E−03 |
| ASMT+ | FST | 1.71E−03 |
| ASMT+ | NSUN2 | 1.91E−03 |
| ASMT+ | THBS4 | 2.29E−03 |
| ASMT+ | RP11-634B7.4 | 6.89E−03 |
| Adipose | ACACB | 0.00E+00 |
| Adipose | PLIN1 | 2.00E−246 |
| Adipose | AQP7 | 5.28E−209 |
| Adipose | CIDEC | 4.75E−193 |
| Adipose | GHR | 4.82E−187 |
| Adipose | ANXA1 | 1.95E−180 |
| Adipose | EBF1 | 3.31E−179 |
| Adipose | PNPLA2 | 1.76E−174 |
| Adipose | PPARG | 6.21E−164 |
| Adipose | CPM | 1.90E−162 |
| Adipose | PIRT | 5.05E−158 |
| Adipose | GPAM | 8.83E−158 |
| Adipose | NEAT1 | 6.05E−156 |
| Adipose | TMEM132C | 9.50E−155 |
| Adipose | LPL | 3.18E−154 |
| Adipose | SLC7A6 | 3.18E−154 |
| Adipose | FOXO1 | 8.98E−142 |
| Adipose | GPD1 | 3.10E−139 |
| Adipose | PDE3B | 2.22E−137 |
| Adipose | SLC7A6OS | 2.58E−136 |
| Adipose | ACSL1 | 7.97E−135 |
| Adipose | ADIPOQ | 5.05E−134 |
| Adipose | CTA-360L10.1 | 7.92E−130 |
| Adipose | GPC6 | 9.33E−130 |
| Adipose | LIPE | 9.50E−128 |
| Adipose | PRKAR2B | 9.25E−120 |
| Adipose | AGPAT2 | 2.40E−114 |
| Adipose | TXNIP | 5.23E−112 |
| Adipose | FGF1 | 3.88E−111 |
| Adipose | TLN2 | 8.14E−111 |
| Adipose | FKBP5 | 4.88E−110 |
| Adipose | LIPE-AS1 | 2.16E−107 |
| Adipose | CBLB | 1.30E−105 |
| Adipose | MGST1 | 5.69E−104 |
| Adipose | RP11-779O18.3 | 1.35E−100 |
| Adipose | FRMD4A | 1.35E−100 |
| Adipose | MLXIPL | 7.52E−100 |
| Adipose | ZNF318 | 1.00E−98 |
| Adipose | RP11-295P9.3 | 2.43E−97 |
| Adipose | ABHD5 | 1.01E−96 |
| Adipose | MARC1 | 1.62E−94 |
| Adipose | SLC1A3 | 5.36E−93 |
| Adipose | SLC19A3 | 9.20E−93 |
| Adipose | GYG2 | 1.15E−92 |
| Adipose | HOOK2 | 1.41E−92 |
| Adipose | PLIN4 | 6.83E−92 |
| Adipose | VIM | 8.45E−91 |
| Adipose | KCNIP2-AS1 | 1.12E−90 |
| Adipose | ADH1B | 3.62E−90 |
| Adipose | EMP1 | 1.12E−88 |
| Adipose | FABP4 | 3.74E−88 |
| Adipose | CD36 | 1.72E−87 |
| Adipose | JUN | 1.72E−87 |
| Adipose | SVEP1 | 4.99E−86 |
| Adipose | ROCK2 | 5.97E−86 |
| Adipose | MMP19 | 4.50E−85 |
| Adipose | FOSB | 1.52E−82 |
| Adipose | TNFAIP8 | 2.33E−81 |
| Adipose | DUSP1 | 3.93E−81 |
| Adipose | ZFP36 | 9.58E−81 |
| Adipose | RHOBTB3 | 1.95E−79 |
| Adipose | HSPB7 | 2.43E−75 |
| Adipose | ACVR1C | 1.58E−74 |
| Adipose | SAT1 | 5.48E−74 |
| Adipose | GPX3 | 1.81E−72 |
| Adipose | RP11-701P16.2 | 3.75E−71 |
| Adipose | MT1X | 4.70E−71 |
| Adipose | SCD | 9.11E−70 |
| Adipose | PALMD | 1.51E−69 |
| Adipose | BCL2 | 1.80E−69 |
| Adipose | PTPRG | 2.86E−68 |
| Adipose | COL4A1 | 1.00E−67 |
| Adipose | RP11-124N14.3 | 1.58E−66 |
| Adipose | RP11-353M9.1 | 2.28E−65 |
| Adipose | ITSN1 | 2.83E−65 |
| Adipose | PHLDB1 | 4.49E−65 |
| Adipose | EPHA1-AS1 | 1.36E−64 |
| Adipose | PLXNA4 | 1.43E−64 |
| Adipose | SIK2 | 1.43E−64 |
| Adipose | G0S2 | 3.17E−63 |
| Adipose | RP11-64D24.2 | 1.77E−62 |
| Adipose | SAA1 | 2.93E−62 |
| Adipose | ANGPTL4 | 1.27E−61 |
| Adipose | C6 | 1.54E−61 |
| Adipose | LAMB1 | 5.30E−61 |
| Adipose | RBMS3-AS3 | 1.24E−60 |
| Adipose | RP11-286B14.1 | 1.59E−60 |
| Adipose | C14orf180 | 7.34E−60 |
| Adipose | COL4A2 | 9.20E−60 |
| Adipose | LIMA1 | 7.28E−59 |
| Adipose | RP11-444D3.1 | 2.65E−58 |
| Adipose | RP11-665G4.1 | 8.59E−58 |
| Adipose | ASPH | 9.93E−58 |
| Adipose | AP000304.12 | 2.82E−57 |
| Adipose | FNDC3B | 1.86E−56 |
| Adipose | GPT | 2.52E−56 |
| Adipose | ACSS3 | 7.60E−56 |
| Adipose | GBE1 | 1.78E−55 |
| Adipose | PFKFB3 | 7.79E−55 |
| Adipose | FASN | 1.08E−54 |
| Adipose | LGALS12 | 2.74E−54 |
| Adipose | RASD1 | 5.65E−54 |
| Adipose | FZD4 | 4.09E−53 |
| Adipose | SLC7A10 | 1.19E−49 |
| Adipose | CIDEA | 1.42E−49 |
| Adipose | HEPN1 | 1.77E−49 |
| Adipose | APCDD1 | 1.04E−44 |
| Adipose | COX14 | 2.59E−43 |
| Adipose | PCK1 | 1.60E−41 |
| Adipose | RP1-193H18.3 | 9.96E−40 |
| Adipose | RGCC | 2.29E−39 |
| Adipose | CDO1 | 1.57E−38 |
| Adipose | GABRE | 8.99E−37 |
| Adipose | NIPSNAP3B | 1.70E−36 |
| Adipose | KCNIP2 | 1.95E−36 |
| Adipose | RP11-563P16.1 | 2.38E−35 |
| Adipose | AKR1C2 | 1.13E−34 |
| Adipose | GPC6-AS1 | 2.25E−32 |
| Adipose | DGAT2 | 8.50E−31 |
| Adipose | RP11-157I4.4 | 4.75E−30 |
| Adipose | ZNF117 | 1.32E−27 |
| Adipose | MT1M | 6.34E−27 |
| Adipose | RP11-111E14.1 | 1.03E−25 |
| Adipose | RBP4 | 5.14E−24 |
| Adipose | ADRA2A | 4.26E−23 |
| Adipose | TIMP4 | 4.48E−23 |
| Adipose | PSG8 | 7.03E−22 |
| Adipose | RP11-286N3.2 | 1.02E−21 |
| Adipose | PFKFB1 | 1.36E−21 |
| Adipose | ID4 | 1.37E−21 |
| Adipose | RP11-154B12.3 | 2.76E−21 |
| Adipose | AZGP1 | 2.96E−21 |
| Adipose | PSG4 | 1.60E−20 |
| Adipose | FNDC4 | 7.36E−20 |
| Adipose | RP11-161D15.3 | 1.52E−19 |
| Adipose | CTB-43E15.3 | 3.98E−18 |
| Adipose | SPATA9 | 6.43E−17 |
| Adipose | MT1A | 2.12E−15 |
| Adipose | HEPACAM | 2.55E−15 |
| Adipose | AMOTL2 | 1.48E−14 |
| Adipose | TM7SF2 | 1.92E−14 |
| Adipose | VEGFA | 6.80E−14 |
| Adipose | MDFI | 9.42E−14 |
| Adipose | CTD-2363C16.1 | 5.89E−13 |
| Adipose | KLHL31 | 4.65E−12 |
| Adipose | RP11-16N2.1 | 1.75E−11 |
| Adipose | RP11-295P9.8 | 2.51E−11 |
| Adipose | CNTFR | 7.29E−11 |
| Adipose | ORMDL3 | 1.20E−10 |
| Adipose | RP11-511B23.3 | 1.36E−10 |
| Adipose | NPY1R | 8.76E−10 |
| Adipose | MOCS1 | 9.56E−10 |
| Adipose | SPRY4 | 6.41E−09 |
| Adipose | APOL4 | 8.79E−08 |
| Epithelial | PHGR1 | 2.68E−238 |
| Epithelial | FXYD3 | 2.68E−238 |
| Epithelial | SLC26A3 | 2.56E−226 |
| Epithelial | ELF3 | 4.55E−192 |
| Epithelial | TSPAN1 | 5.93E−189 |
| Epithelial | PIGR | 2.79E−185 |
| Epithelial | MS4A12 | 9.60E−179 |
| Epithelial | LGALS4 | 5.97E−174 |
| Epithelial | HHLA2 | 1.00E−165 |
| Epithelial | SLC26A2 | 9.34E−162 |
| Epithelial | LIPH | 1.25E−155 |
| Epithelial | CDHR5 | 8.42E−154 |
| Epithelial | MUC12 | 5.27E−153 |
| Epithelial | KRT19 | 2.29E−150 |
| Epithelial | FABP1 | 2.74E−142 |
| Epithelial | SYTL2 | 2.55E−136 |
| Epithelial | SHROOM3 | 9.20E−132 |
| Epithelial | NR3C2 | 1.39E−125 |
| Epithelial | MYO1D | 2.22E−124 |
| Epithelial | RP11-665N17.4 | 2.79E−124 |
| Epithelial | CEACAM1 | 1.46E−122 |
| Epithelial | CEACAM7 | 1.75E−122 |
| Epithelial | SATB2 | 2.76E−119 |
| Epithelial | LGALS3 | 2.15E−116 |
| Epithelial | PPARG | 2.49E−116 |
| Epithelial | KRT20 | 7.43E−115 |
| Epithelial | GUCA2A | 4.25E−114 |
| Epithelial | TMEM45B | 4.50E−112 |
| Epithelial | TCF7L2 | 9.59E−107 |
| Epithelial | BTNL8 | 3.51E−106 |
| Epithelial | HSD11B2 | 3.11E−105 |
| Epithelial | SLC17A4 | 9.97E−105 |
| Epithelial | FRYL | 2.61E−103 |
| Epithelial | HNF4A | 4.82E−102 |
| Epithelial | PLAC8 | 1.91E−101 |
| Epithelial | CDH17 | 2.70E−101 |
| Epithelial | SDCBP2 | 1.89E−100 |
| Epithelial | LLGL2 | 2.36E−100 |
| Epithelial | SELENBP1 | 7.55E−99 |
| Epithelial | AMN | 2.24E−98 |
| Epithelial | PIP5K1B | 2.24E−97 |
| Epithelial | TMPRSS2 | 4.07E−97 |
| Epithelial | PCK1 | 5.11E−97 |
| Epithelial | KLF5 | 1.19E−96 |
| Epithelial | PRSS3 | 1.40E−96 |
| Epithelial | GDA | 7.30E−96 |
| Epithelial | LINC00511 | 2.02E−94 |
| Epithelial | EPCAM | 1.48E−93 |
| Epithelial | ATP1A1 | 2.12E−92 |
| Epithelial | SGK2 | 4.97E−92 |
| Epithelial | CEA | 7.04E−92 |
| Epithelial | MUC13 | 1.32E−91 |
| Epithelial | TMPRSS4 | 1.47E−91 |
| Epithelial | MT-CO1 | 2.42E−91 |
| Epithelial | LINC00278 | 1.57E−90 |
| Epithelial | PLS1 | 2.71E−90 |
| Epithelial | BCAS1 | 3.91E−90 |
| Epithelial | MYH14 | 1.25E−89 |
| Epithelial | GCNT3 | 8.51E−89 |
| Epithelial | TRIM31-AS1 | 9.19E−89 |
| Epithelial | ABCC3 | 3.22E−87 |
| Epithelial | TMIGD1 | 1.34E−86 |
| Epithelial | GPA33 | 2.63E−86 |
| Epithelial | MGLL | 2.74E−86 |
| Epithelial | MT-CO2 | 4.98E−86 |
| Epithelial | CLCA4 | 1.63E−85 |
| Epithelial | PIGZ | 3.52E−84 |
| Epithelial | EZR | 4.66E−84 |
| Epithelial | PAG1 | 2.43E−83 |
| Epithelial | CA4 | 2.76E−83 |
| Epithelial | MYO1E | 2.90E−83 |
| Epithelial | NEDD4L | 2.99E−83 |
| Epithelial | FAM3D | 1.69E−81 |
| Epithelial | DHRS9 | 2.81E−80 |
| Epithelial | CLDN3 | 4.40E−80 |
| Epithelial | AGR3 | 1.13E−79 |
| Epithelial | TINAG | 1.21E−79 |
| Epithelial | ST14 | 1.10E−78 |
| Epithelial | CA12 | 3.81E−78 |
| Epithelial | RP11-747D18.1 | 4.74E−78 |
| Epithelial | HDHD3 | 1.04E−77 |
| Epithelial | GRAMD3 | 1.45E−77 |
| Epithelial | NXPE1 | 1.78E−77 |
| Epithelial | SLC4A4 | 6.02E−77 |
| Epithelial | MT-CO3 | 5.80E−76 |
| Epithelial | CXADR | 1.06E−74 |
| Epithelial | ITM2C | 1.49E−74 |
| Epithelial | PDE3A | 1.31E−73 |
| Epithelial | MXD1 | 7.57E−73 |
| Epithelial | MAST2 | 8.24E−73 |
| Epithelial | SLC44A4 | 3.04E−72 |
| Epithelial | TFCP2L1 | 1.14E−71 |
| Epithelial | RBM47 | 1.96E−71 |
| Epithelial | KRT18 | 3.48E−71 |
| Epithelial | ACSS2 | 3.67E−71 |
| Epithelial | KRT8 | 2.57E−70 |
| Epithelial | SCNN1B | 7.73E−70 |
| Epithelial | PRSS8 | 1.40E−69 |
| Epithelial | PPARGC1A | 6.97E−69 |
| Epithelial | B3GALT5 | 2.92E−68 |
| Epithelial | PDZD3 | 6.35E−67 |
| Epithelial | AQP8 | 1.00E−66 |
| Epithelial | EPS8L3 | 2.54E−66 |
| Epithelial | TMEM54 | 3.86E−66 |
| Epithelial | SERINC2 | 3.84E−64 |
| Epithelial | CCDC64B | 1.95E−59 |
| Epithelial | RASSF7 | 4.58E−58 |
| Epithelial | MTMR11 | 3.65E−57 |
| Epithelial | TMEM171 | 6.61E−57 |
| Epithelial | FAM132A | 7.20E−57 |
| Epithelial | VSIG2 | 2.98E−56 |
| Epithelial | LYPD8 | 1.75E−54 |
| Epithelial | TFF3 | 2.92E−53 |
| Epithelial | CTD-2228K2.5 | 6.91E−53 |
| Epithelial | TRIM31 | 8.16E−52 |
| Epithelial | MALL | 2.04E−50 |
| Epithelial | RP11-396O20.2 | 6.09E−46 |
| Epithelial | C19orf33 | 3.16E−45 |
| Epithelial | COL17A1 | 3.65E−44 |
| Epithelial | ENTPD8 | 2.03E−41 |
| Epithelial | GUCA2B | 3.50E−41 |
| Epithelial | MEP1A | 6.29E−41 |
| Epithelial | HMGCS2 | 1.31E−38 |
| Epithelial | USH1C | 3.21E−38 |
| Epithelial | VIPR1 | 9.98E−38 |
| Epithelial | FUT3 | 6.97E−37 |
| Epithelial | DHRS11 | 1.02E−36 |
| Epithelial | C10orf99 | 2.23E−36 |
| Epithelial | NXPE4 | 1.73E−35 |
| Epithelial | PRR15L | 6.12E−34 |
| Epithelial | TFF1 | 8.37E−34 |
| Epithelial | GPT | 5.88E−33 |
| Epithelial | ARHGEF16 | 6.52E−33 |
| Epithelial | ZG16 | 9.63E−32 |
| Epithelial | CDKN2B | 1.99E−31 |
| Epithelial | MUC2 | 7.66E−31 |
| Epithelial | RP11-357H14.17 | 6.65E−30 |
| Epithelial | SMIM5 | 7.17E−30 |
| Epithelial | RP5-1185I7.1 | 9.18E−30 |
| Epithelial | PRR15 | 2.53E−27 |
| Epithelial | ARL14 | 6.13E−25 |
| Epithelial | RP11-59E19.1 | 1.01E−24 |
| Epithelial | CHP2 | 2.78E−22 |
| Epithelial | LGALS9C | 4.22E−22 |
| Epithelial | AC009133.14 | 2.65E−21 |
| Epithelial | C12orf36 | 3.10E−20 |
| Epithelial | AC024592.9 | 6.00E−20 |
| Epithelial | APOBR | 2.31E−19 |
| Epithelial | RP11-567C2.1 | 5.56E−19 |
| Epithelial | CLDN23 | 6.91E−19 |
| Epithelial | DOK4 | 4.61E−18 |
| Epithelial | RP11-465B22.8 | 5.73E−18 |
| Epithelial | C11orf86 | 1.49E−17 |
| Epithelial | C6orf222 | 1.30E−16 |
| Epithelial | FAM110C | 1.51E−16 |
| Epithelial | TRIM15 | 2.17E−16 |
| Epithelial | CLDN8 | 4.14E−16 |
| Epithelial | FRMD1 | 4.37E−16 |
| Epithelial | SLC6A19 | 3.11E−15 |
| Epithelial | IL2RG | 8.71E−15 |
| Epithelial | MYO1A | 1.94E−14 |
| Epithelial | PRAP1 | 2.99E−14 |
| Epithelial | MUC3A | 7.24E−14 |
| Epithelial | FGFR3 | 9.27E−14 |
| Epithelial | HEPACAM2 | 1.75E−13 |
| Fibroblast | LAMA2 | 1.87E−210 |
| Fibroblast | DCN | 4.37E−201 |
| Fibroblast | ABCA6 | 7.85E−154 |
| Fibroblast | PID1 | 4.25E−144 |
| Fibroblast | RP4-678D15.1 | 1.44E−112 |
| Fibroblast | GPC6 | 5.31E−105 |
| Fibroblast | LINC00478 | 7.92E−103 |
| Fibroblast | EBF1 | 1.04E−97 |
| Fibroblast | FBN1 | 9.48E−94 |
| Fibroblast | RP11-14N7.2 | 9.48E−94 |
| Fibroblast | DLC1 | 6.92E−91 |
| Fibroblast | TSHZ2 | 4.36E−89 |
| Fibroblast | PLXDC2 | 5.19E−87 |
| Fibroblast | MGP | 1.39E−82 |
| Fibroblast | SLIT2 | 3.53E−82 |
| Fibroblast | DCLK1 | 3.53E−82 |
| Fibroblast | RORA | 2.54E−78 |
| Fibroblast | RBMS3 | 1.55E−76 |
| Fibroblast | MFAP5 | 1.51E−71 |
| Fibroblast | MEG3 | 2.19E−70 |
| Fibroblast | ABCA8 | 3.41E−63 |
| Fibroblast | FBLN1 | 3.27E−58 |
| Fibroblast | KAZN | 4.47E−58 |
| Fibroblast | RBMS3-AS3 | 3.71E−56 |
| Fibroblast | AC005237.4 | 2.69E−55 |
| Fibroblast | RP11-39M21.1 | 1.13E−54 |
| Fibroblast | ADH1B | 2.56E−54 |
| Fibroblast | DPT | 1.67E−53 |
| Fibroblast | C1orf21 | 3.11E−53 |
| Fibroblast | COL5A2 | 8.10E−52 |
| Fibroblast | CBLB | 3.43E−51 |
| Fibroblast | TNXB | 6.39E−50 |
| Fibroblast | UAP1 | 1.37E−48 |
| Fibroblast | ACKR3 | 3.29E−48 |
| Fibroblast | C7 | 1.09E−47 |
| Fibroblast | LHFP | 8.37E−47 |
| Fibroblast | NEGR1 | 1.82E−46 |
| Fibroblast | GFPT2 | 1.34E−43 |
| Fibroblast | ABCA9 | 1.12E−41 |
| Fibroblast | IGFBP6 | 2.20E−40 |
| Fibroblast | NFIA | 7.88E−40 |
| Fibroblast | PLCB1 | 1.48E−37 |
| Fibroblast | COL1A2 | 1.33E−36 |
| Fibroblast | ADAMTS5 | 4.04E−36 |
| Fibroblast | SH3PXD2B | 4.08E−36 |
| Fibroblast | LPAR1 | 1.18E−35 |
| Fibroblast | COL3A1 | 2.44E−34 |
| Fibroblast | ZBTB16 | 2.60E−34 |
| Fibroblast | RERG | 7.23E−34 |
| Fibroblast | SVEP1 | 1.22E−32 |
| Fibroblast | FABP6 | 2.00E−32 |
| Fibroblast | KCNN3 | 2.23E−32 |
| Fibroblast | GALNT15 | 7.53E−32 |
| Fibroblast | BICC1 | 1.55E−31 |
| Fibroblast | RHOBTB3 | 1.55E−31 |
| Fibroblast | ADD3 | 1.29E−30 |
| Fibroblast | CFD | 5.92E−30 |
| Fibroblast | AC007319.1 | 6.72E−30 |
| Fibroblast | SDK1 | 2.76E−29 |
| Fibroblast | STEAP2 | 6.07E−29 |
| Fibroblast | LSP1 | 1.07E−28 |
| Fibroblast | LAMB1 | 3.27E−28 |
| Fibroblast | TSC22D3 | 6.61E−28 |
| Fibroblast | NOX4 | 6.97E−28 |
| Fibroblast | TGFBR3 | 9.37E−28 |
| Fibroblast | PI16 | 1.69E−27 |
| Fibroblast | FKBP5 | 1.96E−27 |
| Fibroblast | SPON2 | 9.15E−27 |
| Fibroblast | RP11-64D24.2 | 9.19E−27 |
| Fibroblast | SCARA5 | 3.37E−26 |
| Fibroblast | SLIT3 | 5.62E−26 |
| Fibroblast | EXT1 | 1.20E−25 |
| Fibroblast | MEDAG | 1.33E−25 |
| Fibroblast | MAML2 | 1.33E−25 |
| Fibroblast | PRR16 | 1.80E−25 |
| Fibroblast | FOXO3 | 3.65E−25 |
| Fibroblast | LAMC1 | 5.12E−25 |
| Fibroblast | SRPX2 | 8.17E−25 |
| Fibroblast | FSTL1 | 2.21E−24 |
| Fibroblast | EGR1 | 2.47E−24 |
| Fibroblast | CILP | 4.22E−24 |
| Fibroblast | LUM | 6.92E−24 |
| Fibroblast | F3 | 1.27E−23 |
| Fibroblast | LRRC16A | 1.27E−23 |
| Fibroblast | PLA2G2A | 1.37E−23 |
| Fibroblast | GSN | 1.71E−23 |
| Fibroblast | VCAN | 2.98E−23 |
| Fibroblast | MAPK10 | 7.96E−23 |
| Fibroblast | AOX1 | 1.19E−22 |
| Fibroblast | CCDC80 | 1.71E−22 |
| Fibroblast | FAM65C | 1.73E−22 |
| Fibroblast | SCN7A | 1.85E−22 |
| Fibroblast | CREB5 | 1.90E−22 |
| Fibroblast | PDGFRB | 2.09E−22 |
| Fibroblast | GPRC5A | 4.05E−21 |
| Fibroblast | RP11-597D13.9 | 4.73E−21 |
| Fibroblast | PIK3R1 | 7.88E−21 |
| Fibroblast | RP11-219B17.1 | 1.14E−20 |
| Fibroblast | FMNL2 | 1.27E−20 |
| Fibroblast | PLEKHA5 | 1.49E−20 |
| Fibroblast | MEG8 | 5.79E−20 |
| Fibroblast | VIPR2 | 6.11E−20 |
| Fibroblast | TEX26-AS1 | 2.68E−19 |
| Fibroblast | NRK | 4.79E−19 |
| Fibroblast | PCOLCE | 8.78E−18 |
| Fibroblast | SLC5A9 | 2.05E−17 |
| Fibroblast | RP11-99J16_A.2 | 6.49E−17 |
| Fibroblast | PDGFRA | 1.15E−16 |
| Fibroblast | RP11-13N12.2 | 3.39E−16 |
| Fibroblast | RP11-399D6.2 | 7.45E−16 |
| Fibroblast | THBS2 | 4.93E−15 |
| Fibroblast | CXXC5 | 4.98E−15 |
| Fibroblast | MFGE8 | 8.98E−15 |
| Fibroblast | HAS2-AS1 | 2.98E−14 |
| Fibroblast | MCOLN3 | 3.52E−14 |
| Fibroblast | CYP4A22-AS1 | 5.18E−14 |
| Fibroblast | C1R | 1.29E−13 |
| Fibroblast | SFRP2 | 1.64E−13 |
| Fibroblast | CYP4Z1 | 4.13E−13 |
| Fibroblast | MMP2 | 8.88E−13 |
| Fibroblast | RP11-66B24.4 | 1.08E−12 |
| Fibroblast | RP11-201E8.1 | 1.25E−12 |
| Fibroblast | AL132709.5 | 3.79E−12 |
| Fibroblast | SERPINF1 | 2.04E−11 |
| Fibroblast | PRRX1 | 2.26E−11 |
| Fibroblast | ADAMTS16 | 2.53E−11 |
| Fibroblast | CYP4X1 | 2.86E−11 |
| Fibroblast | RP11-15M15.2 | 8.89E−11 |
| Fibroblast | GPNMB | 1.41E−10 |
| Fibroblast | CD34 | 2.09E−10 |
| Fibroblast | HTRA3 | 2.13E−10 |
| Fibroblast | HAS2 | 2.60E−10 |
| Fibroblast | PAK3 | 2.85E−10 |
| Fibroblast | CXCL12 | 8.30E−10 |
| Fibroblast | ADAMTS15 | 9.24E−10 |
| Fibroblast | MMP19 | 1.24E−09 |
| Fibroblast | RP11-554D13.1 | 1.72E−09 |
| Fibroblast | TPBG | 3.19E−09 |
| Fibroblast | NYNRIN | 4.54E−09 |
| Fibroblast | ITGA11 | 4.58E−09 |
| Fibroblast | INSRR | 5.95E−09 |
| Fibroblast | MMP23B | 1.63E−08 |
| Fibroblast | ADM | 4.26E−08 |
| Fibroblast | AP001172.2 | 4.98E−08 |
| Fibroblast | CYP4B1 | 1.17E−07 |
| Fibroblast | C10orf55 | 1.74E−07 |
| Fibroblast | C4orf17 | 2.52E−07 |
| Fibroblast | AC079742.4 | 3.96E−07 |
| Fibroblast | RP4-530I15.6 | 7.85E−07 |
| Fibroblast | SHC3 | 1.09E−06 |
| Fibroblast | ABCA9-AS1 | 9.11E−06 |
| Glia | CDH19 | 0.00E+00 |
| Glia | BAI3 | 0.00E+00 |
| Glia | PPP2R2B | 8.93E−246 |
| Glia | CADM2 | 2.13E−229 |
| Glia | NRXN3 | 1.09E−221 |
| Glia | NRXN1 | 1.22E−157 |
| Glia | ANGPTL1 | 5.88E−154 |
| Glia | ABCA8 | 4.94E−144 |
| Glia | SHISA9 | 3.53E−139 |
| Glia | ANK2 | 1.66E−128 |
| Glia | NKAIN3 | 1.77E−128 |
| Glia | ANK3 | 2.03E−128 |
| Glia | XKR4 | 1.93E−121 |
| Glia | CHL1 | 5.42E−117 |
| Glia | RALGPS2 | 6.33E−116 |
| Glia | SORCS1 | 1.62E−114 |
| Glia | LRRTM4 | 3.34E−112 |
| Glia | PRIMA1 | 4.70E−112 |
| Glia | RP11-242P2.1 | 1.40E−110 |
| Glia | EPB41L2 | 2.00E−108 |
| Glia | SLC35F1 | 1.24E−105 |
| Glia | GINS3 | 1.09E−104 |
| Glia | FRMD5 | 1.11E−93 |
| Glia | ZNF536 | 1.44E−93 |
| Glia | NKAIN2 | 1.29E−92 |
| Glia | PTPRZ1 | 3.34E−92 |
| Glia | LSAMP | 2.11E−90 |
| Glia | LGI4 | 5.04E−88 |
| Glia | DOCK5 | 2.45E−87 |
| Glia | WDR86 | 2.90E−84 |
| Glia | QKI | 5.57E−84 |
| Glia | BCL2 | 8.33E−83 |
| Glia | AP000462.2 | 2.19E−82 |
| Glia | ASAP2 | 9.12E−81 |
| Glia | RP3-525N10.2 | 2.76E−77 |
| Glia | CTNND2 | 3.14E−77 |
| Glia | LPHN3 | 2.16E−75 |
| Glia | RP11-242P2.2 | 1.53E−74 |
| Glia | LRRTM3 | 2.66E−71 |
| Glia | KIAA1217 | 2.36E−70 |
| Glia | SYT10 | 4.99E−70 |
| Glia | KIRREL3 | 5.38E−70 |
| Glia | KCNMB4 | 6.81E−68 |
| Glia | NCAM1 | 2.32E−67 |
| Glia | CASC14 | 4.54E−67 |
| Glia | LPAR1 | 1.91E−66 |
| Glia | CADM1 | 1.64E−65 |
| Glia | SAMHD1 | 5.44E−65 |
| Glia | LINC00478 | 5.36E−64 |
| Glia | HMCN1 | 5.05E−63 |
| Glia | GRIK3 | 1.28E−62 |
| Glia | HAND2-AS1 | 1.62E−61 |
| Glia | CTNNA3 | 1.35E−60 |
| Glia | POLR2F | 1.17E−55 |
| Glia | SGIP1 | 1.36E−55 |
| Glia | PRKCA | 1.11E−54 |
| Glia | MEG3 | 6.08E−53 |
| Glia | SOX6 | 7.65E−53 |
| Glia | TSPAN11 | 2.63E−52 |
| Glia | HAND2 | 1.06E−51 |
| Glia | COL28A1 | 3.70E−51 |
| Glia | AQP4-AS1 | 1.11E−50 |
| Glia | GPM6B | 5.10E−50 |
| Glia | MARCH10 | 5.51E−49 |
| Glia | SEMA3C | 2.28E−48 |
| Glia | SAT1 | 3.97E−48 |
| Glia | ST3GAL6 | 1.01E−47 |
| Glia | TRDN | 3.78E−46 |
| Glia | CTD-2544M6.1 | 5.14E−46 |
| Glia | PLCE1 | 1.06E−45 |
| Glia | ZSWIM6 | 1.15E−45 |
| Glia | AC018890.6 | 1.41E−45 |
| Glia | EHBP1 | 2.02E−45 |
| Glia | SCN7A | 2.73E−45 |
| Glia | HIBCH | 1.11E−43 |
| Glia | WIPF1 | 1.55E−43 |
| Glia | NCAM2 | 2.71E−43 |
| Glia | INSC | 5.35E−43 |
| Glia | RP11-308N19.1 | 9.60E−42 |
| Glia | NRG3 | 1.89E−41 |
| Glia | RP11-77K12.4 | 3.36E−41 |
| Glia | COL21A1 | 3.89E−41 |
| Glia | COL18A1 | 1.99E−40 |
| Glia | CD9 | 3.93E−40 |
| Glia | FIGN | 5.69E−40 |
| Glia | RASSF4 | 1.22E−39 |
| Glia | FADS2 | 2.75E−38 |
| Glia | RP11-4F22.2 | 3.18E−37 |
| Glia | GPR155 | 4.45E−37 |
| Glia | COL9A3 | 8.81E−37 |
| Glia | RP11-115C10.1 | 3.94E−36 |
| Glia | SORBS2 | 5.20E−36 |
| Glia | MICALL2 | 8.13E−36 |
| Glia | RP11-532N4.2 | 8.75E−36 |
| Glia | CASC15 | 9.89E−36 |
| Glia | MAPRE2 | 2.41E−35 |
| Glia | KCNH8 | 1.41E−34 |
| Glia | CABLES2 | 2.26E−34 |
| Glia | ATP8A1 | 2.99E−34 |
| Glia | NLGN4X | 3.96E−34 |
| Glia | CA1 | 2.30E−33 |
| Glia | DMKN | 2.61E−32 |
| Glia | ST6GALNAC2 | 1.51E−31 |
| Glia | RP11-18D7.3 | 4.73E−30 |
| Glia | RP11-142M10.2 | 8.19E−30 |
| Glia | GPR126 | 1.35E−29 |
| Glia | PLEKHB1 | 4.58E−29 |
| Glia | CRISPLD1 | 2.16E−27 |
| Glia | GRIK2 | 3.90E−26 |
| Glia | SHC4 | 7.54E−26 |
| Glia | CDH2 | 7.18E−25 |
| Glia | MEGF6 | 2.67E−24 |
| Glia | RP11-45A16.4 | 5.31E−24 |
| Glia | ESM1 | 2.91E−23 |
| Glia | RP11-386G21.1 | 8.84E−23 |
| Glia | RP11-2E17.1 | 3.88E−22 |
| Glia | RP4-663N10.1 | 1.61E−21 |
| Glia | PMEPA1 | 5.55E−21 |
| Glia | RP11-531H8.2 | 5.65E−21 |
| Glia | LINC00327 | 3.04E−20 |
| Glia | PAQR6 | 3.02E−19 |
| Glia | COL11A1 | 3.49E−19 |
| Glia | FXYD3 | 7.20E−18 |
| Glia | S100B | 2.92E−16 |
| Glia | ITGB8 | 6.94E−16 |
| Glia | RLBP1 | 9.94E−16 |
| Glia | RP11-381K20.2 | 1.23E−15 |
| Glia | SLC44A3 | 3.74E−15 |
| Glia | HES1 | 4.72E−15 |
| Glia | GAP43 | 1.12E−14 |
| Glia | CDH6 | 1.22E−13 |
| Glia | WNT16 | 2.07E−13 |
| Glia | RP4-792G4.2 | 2.73E−13 |
| Glia | PLP1 | 4.15E−13 |
| Glia | RP11-391J2.3 | 8.87E−13 |
| Glia | SRCIN1 | 2.17E−12 |
| Glia | ACTR5 | 2.89E−12 |
| Glia | RP11-776H12.1 | 6.02E−12 |
| Glia | KCNK5 | 1.41E−11 |
| Glia | CMTM5 | 1.73E−11 |
| Glia | SOX2 | 2.07E−11 |
| Glia | HSPA1B | 2.13E−11 |
| Glia | RP11-1055B8.3 | 6.41E−11 |
| Glia | FXYD1 | 5.72E−10 |
| Glia | PTGDS | 9.11E−10 |
| Glia | HEPN1 | 9.35E−09 |
| Glia | GPC1 | 6.92E−08 |
| Glia | CTC-255N20.1 | 1.11E−07 |
| Glia | TBX3 | 1.34E−07 |
| Glia | L1CAM | 1.77E−07 |
| Glia | AC090505.4 | 2.27E−07 |
| Glia | RP11-202G11.2 | 2.01E−06 |
| HAS1+ | SOD2 | 3.77E−172 |
| HAS1+ | GFPT2 | 5.27E−171 |
| HAS1+ | RP11-66B24.4 | 6.00E−166 |
| HAS1+ | NAMPT | 2.74E−155 |
| HAS1+ | ALDH1A3 | 2.44E−145 |
| HAS1+ | ACSL4 | 2.68E−134 |
| HAS1+ | C3 | 5.16E−130 |
| HAS1+ | HAS1 | 1.07E−129 |
| HAS1+ | MT2A | 2.01E−129 |
| HAS1+ | TIMP1 | 1.13E−127 |
| HAS1+ | WWC1 | 8.53E−106 |
| HAS1+ | COBL | 3.10E−105 |
| HAS1+ | AC016831.7 | 1.94E−102 |
| HAS1+ | FOSL1 | 5.11E−102 |
| HAS1+ | MCTP2 | 9.38E−101 |
| HAS1+ | NFKBIA | 5.62E−100 |
| HAS1+ | CLDN1 | 5.65E−95 |
| HAS1+ | UGP2 | 4.68E−92 |
| HAS1+ | SAT1 | 1.94E−89 |
| HAS1+ | AP000705.7 | 5.99E−84 |
| HAS1+ | FLRT2 | 8.31E−83 |
| HAS1+ | MIR29A | 1.98E−81 |
| HAS1+ | MT1E | 4.56E−79 |
| HAS1+ | SLC7A2 | 1.12E−78 |
| HAS1+ | TJP2 | 1.25E−78 |
| HAS1+ | KLF6 | 1.94E−78 |
| HAS1+ | GPRC5A | 2.84E−78 |
| HAS1+ | MARCH3 | 4.56E−77 |
| HAS1+ | NABP1 | 5.77E−77 |
| HAS1+ | ERRFI1 | 2.36E−75 |
| HAS1+ | EZR | 1.33E−72 |
| HAS1+ | SLC20A1 | 5.27E−72 |
| HAS1+ | KRT18 | 7.40E−70 |
| HAS1+ | CLIC4 | 1.55E−67 |
| HAS1+ | NTNG1 | 1.88E−67 |
| HAS1+ | UAP1 | 1.01E−64 |
| HAS1+ | CXCL1 | 2.91E−61 |
| HAS1+ | RP11-286E11.1 | 6.08E−60 |
| HAS1+ | HIF1A-AS2 | 2.89E−58 |
| HAS1+ | FAM153B | 2.89E−58 |
| HAS1+ | TNFRSF12A | 5.79E−57 |
| HAS1+ | SOX6 | 2.80E−56 |
| HAS1+ | CCDC64 | 5.32E−55 |
| HAS1+ | HIF1A | 3.19E−54 |
| HAS1+ | PLA2G2A | 3.49E−54 |
| HAS1+ | ID2 | 2.62E−53 |
| HAS1+ | EFNA5 | 1.22E−52 |
| HAS1+ | RP11-434I12.2 | 4.04E−52 |
| HAS1+ | CXCL2 | 6.10E−52 |
| HAS1+ | PHLDB2 | 2.09E−51 |
| HAS1+ | CD55 | 3.70E−50 |
| HAS1+ | RP6-99M1.2 | 5.05E−50 |
| HAS1+ | MAST4 | 2.88E−49 |
| HAS1+ | VCAM1 | 4.73E−49 |
| HAS1+ | NFKB1 | 1.12E−47 |
| HAS1+ | CD200 | 4.22E−47 |
| HAS1+ | MEDAG | 4.30E−47 |
| HAS1+ | SLC39A8 | 1.15E−46 |
| HAS1+ | MAP4K4 | 4.14E−46 |
| HAS1+ | OLR1 | 8.41E−46 |
| HAS1+ | IER3 | 3.01E−45 |
| HAS1+ | LMNA | 4.02E−45 |
| HAS1+ | DUSP1 | 4.72E−45 |
| HAS1+ | HOMER1 | 2.20E−44 |
| HAS1+ | S100A6 | 2.23E−44 |
| HAS1+ | SGMS2 | 2.76E−44 |
| HAS1+ | CRY1 | 3.62E−44 |
| HAS1+ | ATP2B1 | 6.59E−44 |
| HAS1+ | CCNL1 | 7.55E−42 |
| HAS1+ | ICAM1 | 8.08E−42 |
| HAS1+ | KRT19 | 2.04E−41 |
| HAS1+ | ARAP2 | 2.83E−41 |
| HAS1+ | STAT3 | 6.81E−41 |
| HAS1+ | PIM1 | 1.17E−40 |
| HAS1+ | ERN1 | 5.76E−40 |
| HAS1+ | RDH10 | 1.44E−39 |
| HAS1+ | ITPKC | 1.88E−39 |
| HAS1+ | CAMSAP2 | 2.73E−39 |
| HAS1+ | THSD4 | 4.22E−39 |
| HAS1+ | KDM6B | 6.42E−39 |
| HAS1+ | PKHD1L1 | 8.77E−39 |
| HAS1+ | OSMR | 2.26E−38 |
| HAS1+ | TNFSF14 | 6.19E−38 |
| HAS1+ | ZFPM2 | 1.82E−37 |
| HAS1+ | RP11-281P23.2 | 1.95E−37 |
| HAS1+ | MAP3K8 | 2.05E−37 |
| HAS1+ | SRGAP1 | 3.70E−37 |
| HAS1+ | PLCB1 | 1.01E−36 |
| HAS1+ | STEAP2 | 1.35E−36 |
| HAS1+ | RBMS1 | 5.50E−36 |
| HAS1+ | KCTD8 | 1.03E−35 |
| HAS1+ | CTD-2005H7.2 | 1.21E−35 |
| HAS1+ | CA12 | 1.67E−35 |
| HAS1+ | PLAUR | 7.82E−35 |
| HAS1+ | NFKBIZ | 1.55E−34 |
| HAS1+ | RNF24 | 1.91E−34 |
| HAS1+ | ANXA1 | 4.85E−34 |
| HAS1+ | LINC00842 | 3.29E−33 |
| HAS1+ | MIR4435-1HG | 4.28E−33 |
| HAS1+ | CTD-2369P2.5 | 6.42E−33 |
| HAS1+ | TNFRSF21 | 8.83E−33 |
| HAS1+ | PDPN | 2.93E−32 |
| HAS1+ | IL6 | 6.53E−32 |
| HAS1+ | TRIB1 | 2.30E−31 |
| HAS1+ | SERPINB9 | 3.25E−31 |
| HAS1+ | DUSP2 | 5.31E−31 |
| HAS1+ | RP11-716H6.2 | 3.67E−30 |
| HAS1+ | RP11-707A18.1 | 2.28E−29 |
| HAS1+ | CALB2 | 8.91E−27 |
| HAS1+ | MFSD2A | 1.58E−25 |
| HAS1+ | CHI3L1 | 1.62E−25 |
| HAS1+ | MT1M | 8.09E−25 |
| HAS1+ | SLPI | 2.64E−24 |
| HAS1+ | LIF | 4.38E−24 |
| HAS1+ | BNC1 | 3.28E−23 |
| HAS1+ | LINC00152 | 4.74E−23 |
| HAS1+ | MTMR7 | 4.95E−23 |
| HAS1+ | FAM110C | 3.22E−22 |
| HAS1+ | FAM153C | 4.47E−22 |
| HAS1+ | TNFAIP3 | 5.04E−21 |
| HAS1+ | RP11-277B15.2 | 8.47E−21 |
| HAS1+ | CFB | 1.71E−19 |
| HAS1+ | MSLN | 5.58E−18 |
| HAS1+ | RP11-74M11.2 | 9.27E−18 |
| HAS1+ | ZC3H12A | 2.75E−17 |
| HAS1+ | PRG4 | 3.36E−17 |
| HAS1+ | PLCH2 | 3.69E−17 |
| HAS1+ | ITLN1 | 4.12E−17 |
| HAS1+ | ARC | 2.82E−16 |
| HAS1+ | TFPI2 | 2.88E−16 |
| HAS1+ | RP11-404P21.3 | 3.75E−16 |
| HAS1+ | GADD45A | 5.16E−16 |
| HAS1+ | RP11-244K5.8 | 1.81E−15 |
| HAS1+ | RP11-290F20.2 | 4.00E−15 |
| HAS1+ | DAW1 | 7.58E−15 |
| HAS1+ | KLK11 | 2.17E−13 |
| HAS1+ | CLEC4M | 3.71E−13 |
| HAS1+ | IL20 | 9.49E−13 |
| HAS1+ | CARNS1 | 1.15E−12 |
| HAS1+ | SERPINB2 | 5.38E−12 |
| HAS1+ | SMPD3 | 1.30E−11 |
| HAS1+ | RP11-667K14.3 | 2.20E−11 |
| HAS1+ | IL8 | 5.42E−11 |
| HAS1+ | PROCR | 1.03E−10 |
| HAS1+ | CCDC71L | 1.92E−10 |
| HAS1+ | TGM1 | 1.92E−10 |
| HAS1+ | RP5-1022P6.5 | 2.39E−10 |
| HAS1+ | EPS8L1 | 2.74E−09 |
| HAS1+ | HILPDA | 4.08E−09 |
| HAS1+ | LINC00707 | 4.37E−09 |
| HAS1+ | VNN3 | 1.56E−08 |
| HAS1+ | GATA6-AS1 | 1.00E−07 |
| HAS1+ | ISYNA1 | 5.39E−06 |
| ICCs | KCNIP4 | 8.99E−193 |
| ICCs | SGCZ | 1.25E−184 |
| ICCs | ANO1 | 6.43E−138 |
| ICCs | DPP10 | 1.67E−130 |
| ICCs | PTGER3 | 2.94E−126 |
| ICCs | RP11-626H12.3 | 6.51E−122 |
| ICCs | SLC12A2 | 2.09E−120 |
| ICCs | KIT | 8.33E−114 |
| ICCs | KIF26B | 6.61E−106 |
| ICCs | GPC6 | 8.52E−103 |
| ICCs | NRG1 | 8.43E−99 |
| ICCs | IL1RAPL2 | 6.15E−90 |
| ICCs | PDE1A | 1.76E−78 |
| ICCs | FHL2 | 1.55E−72 |
| ICCs | CAPN15 | 4.96E−72 |
| ICCs | CPA6 | 4.58E−69 |
| ICCs | ADAMTSL3 | 1.22E−67 |
| ICCs | LDB2 | 5.45E−65 |
| ICCs | BAI3 | 5.80E−61 |
| ICCs | ETV1 | 1.24E−58 |
| ICCs | RP11-626H12.1 | 8.12E−57 |
| ICCs | RP11-62I21.1 | 1.78E−56 |
| ICCs | CACNB2 | 1.16E−55 |
| ICCs | DPT | 6.73E−55 |
| ICCs | PLCB1 | 2.68E−53 |
| ICCs | UNC13C | 1.44E−52 |
| ICCs | PIEZO2 | 2.66E−52 |
| ICCs | KCND2 | 6.42E−51 |
| ICCs | CDH13 | 2.14E−50 |
| ICCs | PLCL1 | 1.83E−48 |
| ICCs | GHR | 9.75E−48 |
| ICCs | MEIS2 | 4.43E−44 |
| ICCs | GRIA4 | 1.38E−41 |
| ICCs | ABCC4 | 1.27E−39 |
| ICCs | RORA | 4.73E−39 |
| ICCs | OBSCN | 2.11E−38 |
| ICCs | LINC01091 | 1.04E−37 |
| ICCs | TMEM132C | 2.13E−36 |
| ICCs | STRBP | 4.66E−36 |
| ICCs | AFF3 | 3.23E−35 |
| ICCs | TRPC4 | 5.18E−35 |
| ICCs | ENOX1 | 1.45E−34 |
| ICCs | FGF1 | 3.76E−34 |
| ICCs | ZBTB20 | 4.20E−34 |
| ICCs | TCF21 | 6.25E−33 |
| ICCs | DCC | 1.18E−31 |
| ICCs | TOX | 1.23E−31 |
| ICCs | FAM49B | 4.05E−31 |
| ICCs | PDE4C | 4.76E−31 |
| ICCs | RP11-140I24.1 | 1.41E−30 |
| ICCs | PDE3A | 1.53E−30 |
| ICCs | MPPED2 | 1.54E−30 |
| ICCs | RP3-323P13.2 | 3.70E−30 |
| ICCs | FGF12-AS1 | 2.27E−29 |
| ICCs | CTD-2009A10.1 | 3.27E−29 |
| ICCs | FBN1 | 2.14E−28 |
| ICCs | RP11-39M21.1 | 1.36E−27 |
| ICCs | PRKG1 | 1.36E−27 |
| ICCs | RP4-678D15.1 | 4.05E−27 |
| ICCs | PAM | 6.63E−27 |
| ICCs | PRKDC | 9.11E−27 |
| ICCs | PDE3B | 2.19E−26 |
| ICCs | BMPR1B | 3.21E−26 |
| ICCs | ZFHX3 | 3.21E−26 |
| ICCs | ACSS3 | 3.39E−26 |
| ICCs | CDH11 | 3.97E−26 |
| ICCs | CHN2 | 4.33E−26 |
| ICCs | TSHZ2 | 2.85E−25 |
| ICCs | MBOAT2 | 9.37E−25 |
| ICCs | C7 | 1.78E−24 |
| ICCs | SPATS2L | 2.37E−24 |
| ICCs | CUX2 | 3.00E−24 |
| ICCs | MAPK10 | 3.29E−24 |
| ICCs | COL13A1 | 4.40E−24 |
| ICCs | SPRY1 | 9.66E−24 |
| ICCs | LINGO2 | 5.71E−23 |
| ICCs | FRAS1 | 1.81E−22 |
| ICCs | PLAT | 5.15E−22 |
| ICCs | NKX3-2 | 2.78E−21 |
| ICCs | B3GALTL | 3.69E−21 |
| ICCs | CSGALNACT1 | 6.12E−21 |
| ICCs | NFKBIZ | 1.23E−20 |
| ICCs | FGF12 | 1.43E−20 |
| ICCs | GNG2 | 2.49E−20 |
| ICCs | NRP1 | 3.29E−20 |
| ICCs | COL12A1 | 3.88E−20 |
| ICCs | RP11-222A11.1 | 4.27E−20 |
| ICCs | FAP | 4.65E−20 |
| ICCs | AHCYL2 | 5.19E−20 |
| ICCs | TMEM100 | 6.08E−20 |
| ICCs | GUCY1A3 | 7.53E−20 |
| ICCs | RAB11A | 1.49E−19 |
| ICCs | PREX2 | 3.50E−19 |
| ICCs | ARHGAP24 | 3.78E−19 |
| ICCs | NBL1 | 8.47E−19 |
| ICCs | FENDRR | 1.38E−18 |
| ICCs | RP11-396J6.1 | 1.63E−18 |
| ICCs | CA2 | 2.21E−18 |
| ICCs | RP11-778J15.1 | 4.81E−18 |
| ICCs | FANCC | 5.99E−18 |
| ICCs | RP11-556G22.3 | 1.27E−17 |
| ICCs | SOX30 | 6.07E−17 |
| ICCs | RP11-626H12.2 | 9.03E−17 |
| ICCs | SLC24A2 | 6.20E−16 |
| ICCs | FBXO48 | 2.92E−15 |
| ICCs | EYA4 | 3.37E−15 |
| ICCs | PSG8 | 1.33E−14 |
| ICCs | VPS37A | 1.48E−14 |
| ICCs | CTA-360L10.1 | 2.50E−14 |
| ICCs | LIN7A | 3.05E−14 |
| ICCs | SMAD7 | 4.33E−14 |
| ICCs | EFCC1 | 3.70E−13 |
| ICCs | CBR3 | 7.89E−13 |
| ICCs | LRTM1 | 1.16E−12 |
| ICCs | PROM1 | 2.45E−12 |
| ICCs | AC012360.6 | 6.23E−12 |
| ICCs | SPRY4 | 9.42E−12 |
| ICCs | HSPA12B | 1.60E−11 |
| ICCs | MCOLN2 | 2.61E−11 |
| ICCs | AC140912.1 | 3.43E−11 |
| ICCs | ITGA4 | 1.95E−10 |
| ICCs | CTSL | 1.99E−10 |
| ICCs | SYNDIG1L | 4.10E−10 |
| ICCs | DPP4 | 4.63E−10 |
| ICCs | RP11-298D21.1 | 9.66E−10 |
| ICCs | SYTL2 | 1.03E−09 |
| ICCs | SULT1C4 | 1.24E−08 |
| ICCs | TMEM204 | 1.47E−08 |
| ICCs | MEST | 1.61E−08 |
| ICCs | GPC6-AS1 | 3.66E−08 |
| ICCs | IBA57 | 1.26E−07 |
| ICCs | CTD-2313P7.1 | 1.50E−07 |
| ICCs | CTD-3253I12.1 | 3.15E−07 |
| ICCs | NTF3 | 1.07E−06 |
| ICCs | CACNA2D3-AS1 | 1.09E−06 |
| ICCs | CLEC11A | 1.86E−06 |
| ICCs | RP11-473O4.3 | 5.45E−06 |
| ICCs | RP11-391J2.3 | 2.06E−05 |
| ICCs | LRRC3B | 2.15E−05 |
| ICCs | AC072062.3 | 2.41E−05 |
| ICCs | FOXF1 | 3.28E−05 |
| ICCs | LINC00571 | 1.48E−04 |
| ICCs | MCOLN3 | 6.46E−04 |
| MPO+ | BTF3 | 0.00E+00 |
| MPO+ | EEF1B2 | 0.00E+00 |
| MPO+ | GNB2L1 | 0.00E+00 |
| MPO+ | HINT1 | 0.00E+00 |
| MPO+ | RPL14 | 0.00E+00 |
| MPO+ | RPL28 | 0.00E+00 |
| MPO+ | RPL29 | 0.00E+00 |
| MPO+ | RPL35 | 0.00E+00 |
| MPO+ | RPL36AL | 0.00E+00 |
| MPO+ | RPL8 | 0.00E+00 |
| MPO+ | RPS13 | 0.00E+00 |
| MPO+ | RPS15 | 0.00E+00 |
| MPO+ | RPS5 | 0.00E+00 |
| MPO+ | SLC25A5 | 0.00E+00 |
| MPO+ | UBA52 | 0.00E+00 |
| MPO+ | RPL23 | 0.00E+00 |
| MPO+ | SRP14 | 0.00E+00 |
| MPO+ | RPLP0 | 0.00E+00 |
| MPO+ | YBX1 | 0.00E+00 |
| MPO+ | NACA | 0.00E+00 |
| MPO+ | GAPDH | 0.00E+00 |
| MPO+ | RPL15 | 0.00E+00 |
| MPO+ | RPS7 | 0.00E+00 |
| MPO+ | SLC25A6 | 0.00E+00 |
| MPO+ | RPS23 | 0.00E+00 |
| MPO+ | COX4I1 | 0.00E+00 |
| MPO+ | MT-ND2 | 0.00E+00 |
| MPO+ | RPL19 | 0.00E+00 |
| MPO+ | RPL7 | 0.00E+00 |
| MPO+ | RPL35A | 0.00E+00 |
| MPO+ | OAZ1 | 0.00E+00 |
| MPO+ | RPL4 | 0.00E+00 |
| MPO+ | RPS3A | 0.00E+00 |
| MPO+ | RPS11 | 0.00E+00 |
| MPO+ | RPL18 | 0.00E+00 |
| MPO+ | RPS6 | 0.00E+00 |
| MPO+ | RPS14 | 0.00E+00 |
| MPO+ | RPS18 | 0.00E+00 |
| MPO+ | RPL36 | 0.00E+00 |
| MPO+ | RPS12 | 0.00E+00 |
| MPO+ | RPS27A | 0.00E+00 |
| MPO+ | MT-CYB | 0.00E+00 |
| MPO+ | RPL27 | 0.00E+00 |
| MPO+ | SERF2 | 0.00E+00 |
| MPO+ | TPT1 | 0.00E+00 |
| MPO+ | MT-ND1 | 0.00E+00 |
| MPO+ | RPL32 | 0.00E+00 |
| MPO+ | RPL11 | 0.00E+00 |
| MPO+ | RPL12 | 0.00E+00 |
| MPO+ | RPL23A | 0.00E+00 |
| MPO+ | RPS20 | 0.00E+00 |
| MPO+ | CHCHD2 | 0.00E+00 |
| MPO+ | RPL6 | 0.00E+00 |
| MPO+ | ZNF90 | 0.00E+00 |
| MPO+ | RPL7A | 0.00E+00 |
| MPO+ | RPS4X | 0.00E+00 |
| MPO+ | RPS15A | 0.00E+00 |
| MPO+ | RPS8 | 0.00E+00 |
| MPO+ | RPL3 | 0.00E+00 |
| MPO+ | RPS16 | 0.00E+00 |
| MPO+ | RPL30 | 0.00E+00 |
| MPO+ | RPL34 | 0.00E+00 |
| MPO+ | RPS24 | 0.00E+00 |
| MPO+ | RPS25 | 0.00E+00 |
| MPO+ | PPIA | 0.00E+00 |
| MPO+ | RPS2 | 0.00E+00 |
| MPO+ | RPS19 | 0.00E+00 |
| MPO+ | RPS9 | 0.00E+00 |
| MPO+ | MT-ATP6 | 0.00E+00 |
| MPO+ | RPL13A | 0.00E+00 |
| MPO+ | EEF1A1 | 0.00E+00 |
| MPO+ | MT-ND4 | 0.00E+00 |
| MPO+ | RPL10A | 0.00E+00 |
| MPO+ | RPL31 | 0.00E+00 |
| MPO+ | RPLP2 | 0.00E+00 |
| MPO+ | PTMA | 0.00E+00 |
| MPO+ | FTL | 0.00E+00 |
| MPO+ | RPS3 | 0.00E+00 |
| MPO+ | RPL13 | 0.00E+00 |
| MPO+ | RPL27A | 0.00E+00 |
| MPO+ | XPO5 | 0.00E+00 |
| MPO+ | APOO | 0.00E+00 |
| MPO+ | BAIAP2L1 | 0.00E+00 |
| MPO+ | RPLP1 | 0.00E+00 |
| MPO+ | RPL41 | 0.00E+00 |
| MPO+ | TXNRD1 | 0.00E+00 |
| MPO+ | MT-CO2 | 0.00E+00 |
| MPO+ | AMBRA1 | 0.00E+00 |
| MPO+ | RPL10 | 0.00E+00 |
| MPO+ | MT-CO3 | 0.00E+00 |
| MPO+ | NBEAL1 | 0.00E+00 |
| MPO+ | MT-CO1 | 0.00E+00 |
| MPO+ | RPL5 | 0.00E+00 |
| MPO+ | RPL37 | 0.00E+00 |
| MPO+ | H2AFZ | 0.00E+00 |
| MPO+ | LDHB | 0.00E+00 |
| MPO+ | FAU | 0.00E+00 |
| MPO+ | RPL24 | 0.00E+00 |
| MPO+ | RPL37A | 0.00E+00 |
| MPO+ | FTH1 | 0.00E+00 |
| MPO+ | C1QBP | 1.38E−278 |
| MPO+ | STMN1 | 5.28E−275 |
| MPO+ | HMGA1 | 1.11E−265 |
| MPO+ | MRPL23 | 2.51E−264 |
| MPO+ | RPL22L1 | 1.77E−211 |
| MPO+ | GLRX5 | 3.21E−193 |
| MPO+ | LYL1 | 2.34E−182 |
| MPO+ | CKS2 | 1.06E−178 |
| MPO+ | HIST1H4C | 7.52E−165 |
| MPO+ | NPM3 | 3.63E−159 |
| MPO+ | TIMM10 | 1.50E−135 |
| MPO+ | COA4 | 5.78E−135 |
| MPO+ | KIAA0125 | 2.86E−123 |
| MPO+ | MRPS12 | 2.13E−120 |
| MPO+ | PTRHD1 | 2.44E−120 |
| MPO+ | CKS1B | 2.95E−107 |
| MPO+ | AKR7A2 | 2.30E−105 |
| MPO+ | HBD | 2.75E−105 |
| MPO+ | ALKBH7 | 3.99E−100 |
| MPO+ | C19orf77 | 1.23E−86 |
| MPO+ | MARCKSL1 | 1.74E−86 |
| MPO+ | ZWINT | 2.16E−79 |
| MPO+ | MKI67 | 3.55E−79 |
| MPO+ | CDT1 | 9.14E−79 |
| MPO+ | CDK2AP2 | 5.48E−78 |
| MPO+ | FAM212A | 3.47E−76 |
| MPO+ | ICAM3 | 1.06E−66 |
| MPO+ | TK1 | 1.76E−60 |
| MPO+ | H2AFX | 2.05E−58 |
| MPO+ | S1PR4 | 3.60E−58 |
| MPO+ | MPO | 7.03E−57 |
| MPO+ | RP11-354E11.2 | 2.15E−56 |
| MPO+ | FAM26F | 8.89E−54 |
| MPO+ | NRGN | 4.83E−52 |
| MPO+ | KLF1 | 5.60E−52 |
| MPO+ | SMIM1 | 1.33E−50 |
| MPO+ | ALYREF | 7.17E−50 |
| MPO+ | CDKN2D | 5.01E−49 |
| MPO+ | PPBP | 1.69E−48 |
| MPO+ | TMEM60 | 1.92E−47 |
| MPO+ | PF4 | 4.25E−47 |
| MPO+ | SMIM10 | 6.73E−46 |
| MPO+ | STXBP2 | 8.40E−45 |
| MPO+ | EVA1B | 1.90E−44 |
| MPO+ | GP9 | 2.30E−43 |
| MPO+ | TMEM97 | 3.66E−40 |
| MPO+ | EXOSC4 | 1.20E−39 |
| MPO+ | NMB | 2.64E−39 |
| MPO+ | C9orf40 | 1.09E−36 |
| MPO+ | CRYGD | 1.94E−35 |
| MPO+ | HBB | 2.05E−35 |
| MPO+ | CMTM5 | 3.32E−35 |
| MPO+ | CTSG | 2.36E−34 |
| MPO+ | RAC3 | 4.56E−34 |
| MPO+ | RGS18 | 2.70E−33 |
| MPO+ | CCNB1 | 5.07E−33 |
| MPO+ | LINC01003 | 5.83E−32 |
| MPO+ | GAPT | 8.84E−32 |
| MPO+ | CA2 | 1.80E−28 |
| MPO+ | MCM2 | 5.23E−28 |
| MPO+ | ENDOG | 5.44E−28 |
| MPO+ | DEFB1 | 4.01E−26 |
| MPO+ | SPP1 | 1.02E−25 |
| MPO+ | PDZK1IP1 | 1.45E−25 |
| MPO+ | ICAM4 | 8.68E−25 |
| MPO+ | HBQ1 | 1.04E−24 |
| MPO+ | EVI2B | 2.59E−22 |
| MPO+ | LRRC8D | 3.93E−22 |
| MPO+ | FKBP1B | 8.69E−22 |
| MPO+ | NAT8 | 8.98E−22 |
| MPO+ | MT1F | 3.41E−21 |
| MPO+ | APOBEC3B | 5.94E−21 |
| MPO+ | MESP1 | 6.28E−21 |
| MPO+ | SLC25A10 | 1.68E−20 |
| MPO+ | AHSP | 1.80E−20 |
| MPO+ | AVP | 3.82E−20 |
| MPO+ | RNF113A | 4.07E−19 |
| MPO+ | C11orf21 | 4.70E−19 |
| MPO+ | ZMYND19 | 2.25E−18 |
| MPO+ | CISH | 5.57E−18 |
| MPO+ | AC004540.4 | 5.78E−18 |
| MPO+ | POMC | 2.00E−17 |
| MPO+ | HPDL | 4.40E−17 |
| MPO+ | MT1G | 5.12E−17 |
| MPO+ | FXYD2 | 5.60E−17 |
| MPO+ | UGT2B7 | 6.26E−16 |
| MPO+ | CHST13 | 1.45E−14 |
| MPO+ | ALDOB | 5.49E−14 |
| MPO+ | MT1H | 6.69E−13 |
| Mast cells | TPSAB1 | 0.00E+00 |
| Mast cells | CD69 | 2.79E−177 |
| Mast cells | KIT | 9.34E−152 |
| Mast cells | SRGN | 5.37E−122 |
| Mast cells | HPGDS | 3.00E−119 |
| Mast cells | NTM | 6.55E−111 |
| Mast cells | IL18R1 | 1.10E−110 |
| Mast cells | PZP | 3.64E−101 |
| Mast cells | CPM | 4.56E−94 |
| Mast cells | SYTL3 | 2.96E−76 |
| Mast cells | CPA3 | 4.88E−74 |
| Mast cells | RGS13 | 5.03E−70 |
| Mast cells | RP11-680B3.2 | 5.03E−70 |
| Mast cells | ANXA1 | 5.95E−65 |
| Mast cells | VWA5A | 2.68E−63 |
| Mast cells | RGS1 | 1.65E−62 |
| Mast cells | HDC | 2.95E−59 |
| Mast cells | BATF | 3.50E−57 |
| Mast cells | TSC22D3 | 1.11E−54 |
| Mast cells | GATA2 | 5.72E−53 |
| Mast cells | SAMSN1 | 1.20E−51 |
| Mast cells | AP003025.2 | 3.28E−46 |
| Mast cells | RP13-726E6.1 | 1.11E−44 |
| Mast cells | BMP2K | 1.62E−44 |
| Mast cells | SLCO2B1 | 2.22E−43 |
| Mast cells | SLC24A3 | 2.00E−41 |
| Mast cells | RP13-143G15.3 | 6.80E−41 |
| Mast cells | C1orf186 | 9.17E−41 |
| Mast cells | COX16 | 1.17E−40 |
| Mast cells | GLOD5 | 6.98E−39 |
| Mast cells | RHOH | 9.34E−38 |
| Mast cells | RP13-143G15.4 | 9.88E−38 |
| Mast cells | RP11-779O18.3 | 5.83E−37 |
| Mast cells | ZNF107 | 1.45E−36 |
| Mast cells | CTD-3179P9.1 | 1.11E−35 |
| Mast cells | CHN2 | 1.35E−34 |
| Mast cells | LIF | 6.61E−34 |
| Mast cells | RGS2 | 8.52E−34 |
| Mast cells | ARHGAP15 | 2.16E−33 |
| Mast cells | DUSP1 | 2.85E−33 |
| Mast cells | FER | 2.26E−32 |
| Mast cells | ARHGAP18 | 7.21E−32 |
| Mast cells | SGK1 | 1.22E−30 |
| Mast cells | RP11-217L21.1 | 6.82E−30 |
| Mast cells | CUL2 | 7.59E−30 |
| Mast cells | STX3 | 2.09E−28 |
| Mast cells | HPGD | 2.60E−28 |
| Mast cells | TG | 4.30E−28 |
| Mast cells | FOSB | 1.18E−26 |
| Mast cells | PRKX-AS1 | 8.94E−26 |
| Mast cells | SLC8A3 | 9.10E−26 |
| Mast cells | ZEB2 | 3.32E−25 |
| Mast cells | ELMO1-AS1 | 4.06E−25 |
| Mast cells | ACER3 | 1.94E−24 |
| Mast cells | SLC18A2 | 3.14E−24 |
| Mast cells | ANKRD44 | 3.42E−24 |
| Mast cells | FOS | 1.09E−23 |
| Mast cells | NFKBIA | 1.56E−23 |
| Mast cells | RAB11A | 4.27E−23 |
| Mast cells | LINC00937 | 5.34E−23 |
| Mast cells | CDK15 | 7.44E−23 |
| Mast cells | RCSD1 | 8.69E−23 |
| Mast cells | TNIK | 2.57E−22 |
| Mast cells | KIAA1549 | 2.58E−22 |
| Mast cells | FTH1 | 2.61E−22 |
| Mast cells | SLC2A3 | 2.84E−22 |
| Mast cells | CTSG | 1.95E−21 |
| Mast cells | P2RX1 | 2.76E−21 |
| Mast cells | XIST | 6.14E−21 |
| Mast cells | PAQR3 | 8.66E−21 |
| Mast cells | CD44 | 1.03E−20 |
| Mast cells | MIR24-2 | 1.91E−20 |
| Mast cells | RP11-815J21.4 | 6.45E−20 |
| Mast cells | VIM | 3.19E−19 |
| Mast cells | LMNA | 5.06E−19 |
| Mast cells | TESPA1 | 7.45E−18 |
| Mast cells | DOCK10 | 1.11E−17 |
| Mast cells | CPEB4 | 1.18E−17 |
| Mast cells | AKAP13 | 2.18E−17 |
| Mast cells | MAML3 | 2.84E−17 |
| Mast cells | RP11-557H15.4 | 4.96E−17 |
| Mast cells | MCTP2 | 5.86E−17 |
| Mast cells | EIF2B5-AS1 | 7.34E−17 |
| Mast cells | SLC38A11 | 7.34E−17 |
| Mast cells | ALOX5 | 7.34E−17 |
| Mast cells | PRKX | 7.34E−17 |
| Mast cells | MKRN3 | 1.02E−16 |
| Mast cells | LAX1 | 2.14E−16 |
| Mast cells | RP11-347P5.1 | 3.30E−16 |
| Mast cells | PHF20 | 3.42E−16 |
| Mast cells | H3F3B | 1.18E−15 |
| Mast cells | RAB27B | 2.84E−15 |
| Mast cells | TRAF3IP3 | 3.24E−15 |
| Mast cells | RP11-768F21.1 | 3.61E−15 |
| Mast cells | SAT1 | 7.73E−15 |
| Mast cells | SKAP1 | 8.08E−15 |
| Mast cells | AC009313.1 | 9.73E−15 |
| Mast cells | IER2 | 1.23E−14 |
| Mast cells | PARP4 | 1.54E−14 |
| Mast cells | KCNE1 | 1.57E−14 |
| Mast cells | GRAP2 | 1.69E−14 |
| Mast cells | CSF1 | 3.39E−14 |
| Mast cells | RENBP | 3.54E−14 |
| Mast cells | SYTL2 | 3.92E−14 |
| Mast cells | LCP2 | 1.72E−13 |
| Mast cells | AGPAT9 | 2.68E−13 |
| Mast cells | MIR142 | 4.98E−13 |
| Mast cells | TNFAIP3 | 6.51E−13 |
| Mast cells | RP11-406A9.2 | 2.90E−12 |
| Mast cells | CD37 | 4.17E−12 |
| Mast cells | TMC8 | 9.98E−12 |
| Mast cells | MLPH | 5.26E−11 |
| Mast cells | PIK3R6 | 7.39E−11 |
| Mast cells | EMR2 | 7.90E−11 |
| Mast cells | ARHGAP25 | 1.42E−10 |
| Mast cells | MS4A4E | 4.77E−10 |
| Mast cells | ANKRD18B | 5.48E−10 |
| Mast cells | CTD-2197I11.1 | 8.26E−10 |
| Mast cells | IKZF1 | 9.59E−10 |
| Mast cells | CTD-2583P5.1 | 1.89E−09 |
| Mast cells | RP11-456D7.1 | 2.08E−08 |
| Mast cells | RP11-553K8.5 | 3.08E−08 |
| Mast cells | RP11-440I14.2 | 1.17E−07 |
| Mast cells | CD22 | 3.01E−07 |
| Mast cells | NLRP9 | 1.06E−06 |
| Mast cells | CTD-2562J17.2 | 1.31E−06 |
| Mast cells | RP11-179A10.1 | 1.73E−06 |
| Mast cells | RP5-1022J11.2 | 4.93E−06 |
| Mast cells | GPR68 | 5.19E−06 |
| Mast cells | BTK | 1.59E−05 |
| Mast cells | WNT8B | 3.47E−05 |
| Mast cells | CD84 | 5.75E−05 |
| Mast cells | AC007879.1 | 7.56E−05 |
| Mast cells | GALNT3 | 1.12E−04 |
| Mast cells | LINC01094 | 1.99E−04 |
| Mast cells | MIIP | 2.69E−04 |
| Mast cells | FAM196B | 2.96E−04 |
| Mast cells | GAB3 | 3.41E−04 |
| Mast cells | ITGAX | 3.44E−04 |
| Mast cells | LAIR1 | 8.78E−04 |
| Mast cells | EIF2D | 6.32E−03 |
| Mast cells | ALOX5AP | 1.25E−02 |
| NKX2-3+ | PIK3C2G | 4.12E−234 |
| NKX2-3+ | NFIB | 1.53E−211 |
| NKX2-3+ | RP11-499F3.2 | 2.40E−202 |
| NKX2-3+ | TTC6 | 1.73E−190 |
| NKX2-3+ | C8orf4 | 1.57E−187 |
| NKX2-3+ | HLA-B | 9.67E−171 |
| NKX2-3+ | BMP5 | 2.12E−158 |
| NKX2-3+ | HLA-A | 3.50E−138 |
| NKX2-3+ | TMC5 | 4.27E−137 |
| NKX2-3+ | PIGR | 5.67E−134 |
| NKX2-3+ | HMGB3 | 6.02E−129 |
| NKX2-3+ | CXCL17 | 3.50E−128 |
| NKX2-3+ | LINC00669 | 2.86E−120 |
| NKX2-3+ | SDK1 | 1.87E−118 |
| NKX2-3+ | MIR205HG | 7.76E−115 |
| NKX2-3+ | IDO1 | 2.03E−114 |
| NKX2-3+ | WFDC2 | 1.77E−111 |
| NKX2-3+ | SLC26A2 | 3.05E−106 |
| NKX2-3+ | CLIC6 | 7.58E−106 |
| NKX2-3+ | PDE5A | 4.33E−103 |
| NKX2-3+ | BCL2 | 9.39E−103 |
| NKX2-3+ | PLEKHA7 | 1.51E−102 |
| NKX2-3+ | ELF3 | 1.14E−98 |
| NKX2-3+ | CDKN2A | 1.15E−97 |
| NKX2-3+ | SOX4 | 7.17E−97 |
| NKX2-3+ | ALCAM | 1.14E−96 |
| NKX2-3+ | RPS19 | 7.33E−95 |
| NKX2-3+ | CD99L2 | 1.48E−94 |
| NKX2-3+ | PLCZ1 | 3.45E−93 |
| NKX2-3+ | GPR98 | 6.80E−93 |
| NKX2-3+ | MDM4 | 6.78E−92 |
| NKX2-3+ | SNTB1 | 1.98E−91 |
| NKX2-3+ | EYA2 | 1.31E−88 |
| NKX2-3+ | DNAH14 | 1.87E−83 |
| NKX2-3+ | CTD-2034I4.1 | 1.60E−82 |
| NKX2-3+ | L3MBTL4 | 6.10E−82 |
| NKX2-3+ | SYCP2 | 1.05E−79 |
| NKX2-3+ | CA1 | 1.15E−79 |
| NKX2-3+ | NEBL | 1.04E−76 |
| NKX2-3+ | PLEKHA5 | 2.81E−73 |
| NKX2-3+ | RP11-1084J3.4 | 5.71E−71 |
| NKX2-3+ | RP11-25H12.1 | 3.28E−69 |
| NKX2-3+ | KCNB2 | 5.22E−69 |
| NKX2-3+ | SEC11C | 1.01E−68 |
| NKX2-3+ | FRMPD4 | 9.96E−68 |
| NKX2-3+ | RP11-337C18.8 | 1.73E−67 |
| NKX2-3+ | RP11-664H17.1 | 3.69E−67 |
| NKX2-3+ | FMO3 | 5.09E−67 |
| NKX2-3+ | ATP13A3 | 6.56E−67 |
| NKX2-3+ | RP11-69E11.4 | 2.42E−65 |
| NKX2-3+ | NKX2-1 | 2.49E−64 |
| NKX2-3+ | MLPH | 2.00E−63 |
| NKX2-3+ | RP11-120J1.1 | 6.04E−63 |
| NKX2-3+ | GALNT1 | 1.56E−62 |
| NKX2-3+ | ALDH3A2 | 1.93E−62 |
| NKX2-3+ | CDH7 | 2.10E−62 |
| NKX2-3+ | RPLP2 | 5.76E−62 |
| NKX2-3+ | RNMT | 1.67E−61 |
| NKX2-3+ | MECOM | 5.89E−61 |
| NKX2-3+ | HLA-C | 6.25E−61 |
| NKX2-3+ | VEGFA | 1.49E−60 |
| NKX2-3+ | WDR49 | 4.83E−59 |
| NKX2-3+ | SOX2 | 5.81E−59 |
| NKX2-3+ | SFTA3 | 5.81E−59 |
| NKX2-3+ | KCTD8 | 3.82E−58 |
| NKX2-3+ | CP | 8.27E−57 |
| NKX2-3+ | WARS | 1.36E−56 |
| NKX2-3+ | FMR1 | 1.41E−56 |
| NKX2-3+ | RP11-793A3.2 | 2.96E−56 |
| NKX2-3+ | KCNK1 | 3.36E−56 |
| NKX2-3+ | AL589743.1 | 1.17E−55 |
| NKX2-3+ | MDK | 8.66E−55 |
| NKX2-3+ | RPL10 | 5.09E−54 |
| NKX2-3+ | CD74 | 6.64E−54 |
| NKX2-3+ | RP11-361I14.2 | 2.54E−53 |
| NKX2-3+ | AC159540.1 | 2.94E−53 |
| NKX2-3+ | SLC34A2 | 5.45E−53 |
| NKX2-3+ | CEACAM6 | 1.32E−52 |
| NKX2-3+ | RPS27 | 2.47E−52 |
| NKX2-3+ | RP11-638I2.8 | 4.26E−52 |
| NKX2-3+ | ABHD3 | 1.25E−51 |
| NKX2-3+ | SMCHD1 | 2.04E−51 |
| NKX2-3+ | IFI27 | 3.45E−51 |
| NKX2-3+ | GDE1 | 8.45E−51 |
| NKX2-3+ | RERGL | 9.99E−51 |
| NKX2-3+ | FANCL | 2.37E−50 |
| NKX2-3+ | RPLP1 | 2.48E−49 |
| NKX2-3+ | SMC4 | 5.82E−49 |
| NKX2-3+ | RPS23 | 6.16E−49 |
| NKX2-3+ | OOEP | 9.82E−49 |
| NKX2-3+ | NUCKS1 | 1.16E−48 |
| NKX2-3+ | CDKAL1 | 3.79E−48 |
| NKX2-3+ | KLK12 | 6.25E−48 |
| NKX2-3+ | CHODL | 6.70E−48 |
| NKX2-3+ | HES6 | 7.96E−48 |
| NKX2-3+ | GALNTL6 | 1.40E−47 |
| NKX2-3+ | RP11-191L9.4 | 1.43E−47 |
| NKX2-3+ | RPL38 | 2.45E−47 |
| NKX2-3+ | HLA-F | 5.34E−47 |
| NKX2-3+ | TOX3 | 1.46E−46 |
| NKX2-3+ | CLDN3 | 6.73E−44 |
| NKX2-3+ | FOXA1 | 3.94E−42 |
| NKX2-3+ | RDH10 | 7.20E−42 |
| NKX2-3+ | EHF | 1.96E−39 |
| NKX2-3+ | SLFN13 | 2.88E−37 |
| NKX2-3+ | FAM111B | 6.36E−37 |
| NKX2-3+ | E2F1 | 3.46E−36 |
| NKX2-3+ | TFAP2A | 3.15E−33 |
| NKX2-3+ | ASPM | 3.16E−33 |
| NKX2-3+ | PNMA3 | 1.17E−32 |
| NKX2-3+ | PRAME | 1.20E−32 |
| NKX2-3+ | SLITRK6 | 1.89E−32 |
| NKX2-3+ | SLPI | 1.30E−30 |
| NKX2-3+ | KLK11 | 3.43E−30 |
| NKX2-3+ | TCP10L2 | 3.68E−30 |
| NKX2-3+ | CENPK | 1.83E−28 |
| NKX2-3+ | ASCL1 | 7.16E−28 |
| NKX2-3+ | CRNDE | 1.08E−27 |
| NKX2-3+ | PFKFB2 | 4.01E−26 |
| NKX2-3+ | KRT18 | 1.74E−24 |
| NKX2-3+ | AC116614.1 | 2.26E−24 |
| NKX2-3+ | SLC15A5 | 3.95E−24 |
| NKX2-3+ | AC011298.2 | 3.59E−23 |
| NKX2-3+ | KRT7 | 6.11E−23 |
| NKX2-3+ | PLEKHG4B | 4.51E−22 |
| NKX2-3+ | RASD1 | 8.08E−22 |
| NKX2-3+ | FAM84B | 8.93E−21 |
| NKX2-3+ | HTR1F | 2.89E−20 |
| NKX2-3+ | GBP5 | 3.19E−20 |
| NKX2-3+ | HOOK1 | 4.00E−20 |
| NKX2-3+ | SIX1 | 5.17E−20 |
| NKX2-3+ | HOXB7 | 6.97E−20 |
| NKX2-3+ | CNKSR1 | 3.96E−19 |
| NKX2-3+ | CALML5 | 4.43E−19 |
| NKX2-3+ | RP11-357H14.17 | 7.32E−19 |
| NKX2-3+ | PAX9 | 9.20E−19 |
| NKX2-3+ | ONECUT2 | 2.55E−16 |
| NKX2-3+ | RP11-328N19.1 | 7.72E−16 |
| NKX2-3+ | RP11-279F6.3 | 6.77E−15 |
| NKX2-3+ | FGL1 | 7.30E−15 |
| NKX2-3+ | RECQL4 | 1.06E−14 |
| NKX2-3+ | AC096670.3 | 1.28E−14 |
| NKX2-3+ | CNFN | 1.31E−14 |
| NKX2-3+ | GEMIN4 | 7.60E−14 |
| NKX2-3+ | RP1 | 7.61E−14 |
| NKX2-3+ | TMEM30B | 1.27E−13 |
| NKX2-3+ | MIOX | 1.87E−13 |
| NKX2-3+ | COLCA1 | 4.45E−13 |
| NKX2-3+ | RP11-499F3.1 | 7.41E−13 |
| NKX2-3+ | RP11-96D1.11 | 1.93E−12 |
| NKX2-3+ | ZNF275 | 2.01E−12 |
| NKX2-3+ | RP11-683L23.1 | 3.44E−12 |
| NKX2-3+ | SLC6A20 | 4.23E−12 |
| NKX2-3+ | PLEKHG4 | 7.48E−12 |
| NKX2-3+ | GYLTL1B | 1.35E−11 |
| NKX2-3+ | DLL1 | 2.48E−11 |
| NKX2-3+ | GBP4 | 6.70E−11 |
| NKX2-3+ | DUSP26 | 9.29E−11 |
| NKX2-3+ | SPINT1 | 1.97E−10 |
| NKX2-3+ | RAB17 | 2.28E−10 |
| NKX2-3+ | ZNF395 | 1.30E−09 |
| NKX2-3+ | BRPF3 | 1.59E−08 |
| NKX2-3+ | FANCB | 6.66E−08 |
| Neuron | MEG3 | 6.09E−160 |
| Neuron | SYT1 | 5.80E−149 |
| Neuron | UCHL1 | 1.59E−147 |
| Neuron | PRPH | 5.56E−130 |
| Neuron | STMN2 | 5.42E−115 |
| Neuron | MAP1B | 2.69E−113 |
| Neuron | CTNNA2 | 1.71E−110 |
| Neuron | PCSK1N | 6.16E−103 |
| Neuron | KIF21A | 1.91E−97 |
| Neuron | ANK2 | 5.62E−94 |
| Neuron | GAL | 4.31E−90 |
| Neuron | PARM1 | 1.95E−89 |
| Neuron | THY1 | 5.21E−81 |
| Neuron | VIP | 2.29E−79 |
| Neuron | MT-CO1 | 1.18E−78 |
| Neuron | GAP43 | 1.26E−77 |
| Neuron | MT-CO3 | 1.32E−76 |
| Neuron | DSCAM | 7.52E−76 |
| Neuron | TMEM59L | 2.54E−75 |
| Neuron | PCDH9 | 1.20E−73 |
| Neuron | TMEM108 | 2.17E−73 |
| Neuron | MIR137HG | 9.01E−73 |
| Neuron | ELAVL4 | 2.20E−72 |
| Neuron | SNAP25 | 5.29E−71 |
| Neuron | SCG2 | 1.87E−70 |
| Neuron | SNCG | 3.64E−68 |
| Neuron | UNC80 | 1.16E−64 |
| Neuron | ALCAM | 1.22E−64 |
| Neuron | BAI3 | 1.46E−64 |
| Neuron | PTPRN | 4.55E−64 |
| Neuron | KIF1A | 1.39E−63 |
| Neuron | GNG3 | 2.52E−63 |
| Neuron | CHRNA3 | 5.49E−62 |
| Neuron | MLLT11 | 5.49E−62 |
| Neuron | RAB3B | 5.74E−62 |
| Neuron | AC016716.2 | 4.39E−60 |
| Neuron | TAGLN3 | 8.26E−60 |
| Neuron | CADM1 | 1.50E−59 |
| Neuron | PCLO | 7.79E−59 |
| Neuron | HS6ST3 | 6.03E−58 |
| Neuron | YWHAH | 1.59E−57 |
| Neuron | EML5 | 1.89E−57 |
| Neuron | PLEKHA5 | 2.71E−57 |
| Neuron | CNTNAP2 | 1.56E−56 |
| Neuron | MT-CO2 | 1.82E−56 |
| Neuron | NCAM2 | 1.95E−54 |
| Neuron | DKK3 | 3.95E−54 |
| Neuron | MT-CYB | 4.22E−54 |
| Neuron | RALYL | 8.84E−54 |
| Neuron | L1CAM | 1.20E−53 |
| Neuron | BEX1 | 5.98E−53 |
| Neuron | CEND1 | 2.88E−52 |
| Neuron | CAMK4 | 2.60E−51 |
| Neuron | CADPS | 2.62E−51 |
| Neuron | CARTPT | 3.25E−51 |
| Neuron | KIF5C | 5.16E−51 |
| Neuron | S100A6 | 7.32E−51 |
| Neuron | SCN3A | 4.22E−50 |
| Neuron | FAIM2 | 4.80E−50 |
| Neuron | NCAM1 | 6.12E−50 |
| Neuron | KIF5A | 8.67E−50 |
| Neuron | STMN4 | 9.97E−50 |
| Neuron | ARHGAP26 | 9.97E−50 |
| Neuron | RTN1 | 1.51E−49 |
| Neuron | NRSN1 | 7.21E−49 |
| Neuron | RET | 3.96E−48 |
| Neuron | CADM3 | 5.56E−48 |
| Neuron | MEG8 | 5.76E−48 |
| Neuron | CACNA1B | 5.82E−48 |
| Neuron | TUBB2B | 3.72E−47 |
| Neuron | FHOD3 | 9.13E−47 |
| Neuron | SYP | 9.34E−47 |
| Neuron | SCN9A | 1.03E−46 |
| Neuron | CDH2 | 1.03E−46 |
| Neuron | SYT4 | 2.26E−46 |
| Neuron | JAKMIP1 | 2.46E−45 |
| Neuron | PTPRR | 3.99E−45 |
| Neuron | MT-ATP6 | 3.99E−45 |
| Neuron | CTC-548K16.1 | 1.23E−44 |
| Neuron | PCBP3 | 2.29E−44 |
| Neuron | MAP2 | 2.98E−44 |
| Neuron | TPPP3 | 3.29E−44 |
| Neuron | LGALS1 | 3.38E−44 |
| Neuron | DPYSL2 | 1.12E−43 |
| Neuron | TUBA1B | 1.72E−43 |
| Neuron | ENTPD3 | 1.99E−43 |
| Neuron | SPOCK2 | 5.33E−43 |
| Neuron | CALM1 | 1.14E−42 |
| Neuron | NCOA7 | 1.29E−42 |
| Neuron | DCBLD2 | 3.95E−42 |
| Neuron | ATP1B1 | 1.15E−41 |
| Neuron | SGIP1 | 1.95E−41 |
| Neuron | NOS1 | 2.13E−41 |
| Neuron | FGF13 | 3.36E−41 |
| Neuron | LPHN3 | 3.54E−41 |
| Neuron | MIAT | 3.91E−41 |
| Neuron | ADAMTS19 | 4.86E−41 |
| Neuron | KIAA1244 | 6.08E−41 |
| Neuron | MAPIA | 1.27E−40 |
| Neuron | IFI27L2 | 1.28E−40 |
| Neuron | CELF3 | 1.62E−40 |
| Neuron | BEX2 | 2.84E−39 |
| Neuron | TTC9B | 2.06E−37 |
| Neuron | RUNDC3A | 6.72E−37 |
| Neuron | AC008067.2 | 2.36E−36 |
| Neuron | PDIA2 | 2.08E−35 |
| Neuron | CALY | 6.17E−35 |
| Neuron | MAPK8IP2 | 8.52E−35 |
| Neuron | SULT4A1 | 8.96E−33 |
| Neuron | SYNGR3 | 1.52E−32 |
| Neuron | CHGA | 9.44E−31 |
| Neuron | PHOX2B | 1.98E−30 |
| Neuron | TLX2 | 2.63E−30 |
| Neuron | ADCYAP1 | 2.36E−28 |
| Neuron | ODAM | 7.09E−28 |
| Neuron | BEX5 | 1.57E−26 |
| Neuron | SYT5 | 1.71E−26 |
| Neuron | PNMA2 | 4.88E−26 |
| Neuron | GPR22 | 8.44E−25 |
| Neuron | MT3 | 2.76E−24 |
| Neuron | FKBP1B | 4.10E−24 |
| Neuron | LINC00599 | 2.05E−22 |
| Neuron | OGDHL | 3.62E−22 |
| Neuron | GNG8 | 4.08E−21 |
| Neuron | RP11-272L13.3 | 1.42E−20 |
| Neuron | RP11-650L12.2 | 5.16E−20 |
| Neuron | POU3F3 | 1.40E−19 |
| Neuron | NEFM | 2.34E−19 |
| Neuron | VSTM2A | 1.72E−18 |
| Neuron | GCGR | 1.73E−18 |
| Neuron | SLC4A3 | 5.92E−18 |
| Neuron | NAP1L3 | 2.05E−16 |
| Neuron | TAC1 | 4.02E−16 |
| Neuron | PENK | 4.32E−16 |
| Neuron | ST8SIA3 | 5.34E−16 |
| Neuron | KIF26A | 5.36E−16 |
| Neuron | TMEM63C | 7.40E−16 |
| Neuron | TRPA1 | 5.84E−15 |
| Neuron | RP11-490G2.2 | 7.72E−14 |
| Neuron | C12orf68 | 1.74E−13 |
| Neuron | CDK5R2 | 2.75E−13 |
| Neuron | LRRC55 | 5.46E−13 |
| Neuron | NPY | 7.17E−13 |
| Neuron | SLC10A4 | 7.38E−13 |
| Neuron | KCNC1 | 1.38E−12 |
| Neuron | TCEAL5 | 2.64E−12 |
| Neuron | PIANP | 2.82E−12 |
| Neuron | VGF | 4.03E−12 |
| Neuron | GPR42 | 5.40E−12 |
| Neuron | KLHL34 | 2.81E−11 |
| Neuron | FFAR3 | 3.36E−11 |
| Neuron | TUBB4A | 5.84E−11 |
| Neuron | TCEAL6 | 1.08E−10 |
| Neuron | ZDHHC22 | 1.55E−10 |
| Neuron | LINC00086 | 2.12E−10 |
| Neuron | SLC35D3 | 2.25E−10 |
| Neuron | NPY2R | 2.87E−10 |
| Neuron | TMEM151A | 3.56E−10 |
| Neuron | HR | 3.61E−10 |
| Neuron | RPRML | 1.28E−09 |
| Neuron | NAT8L | 1.61E−09 |
| Neuron | CAMK1G | 2.71E−09 |
| Neuron | DIRAS1 | 2.88E−09 |
| Neuron | RP11-98D18.1 | 4.89E−09 |
| Neuron | CPNE6 | 1.09E−08 |
| Neuron | FAM131B | 1.30E−08 |
| Neuron | SCRT1 | 1.87E−08 |
| Neuron | AC079154.1 | 2.50E−08 |
| Neuron | ASIC3 | 3.78E−08 |
| Neuron | RP11-122K13.14 | 5.05E−08 |
| Neuron | RP11-531A24.3 | 6.31E−08 |
| Neuron | RP4-555D20.2 | 6.84E−08 |
| Neuron | CELSR3 | 2.34E−07 |
| Neuron | RP11-284N8.3 | 5.28E−07 |
| Neuron | HMX2 | 6.25E−07 |
| Neuron | CAMK2N2 | 8.21E−07 |
| Neuron | USP35 | 1.42E−06 |
| Neuron | CCDC78 | 1.68E−06 |
| Neuron | HOXD1 | 3.26E−06 |
| Neuron | RP11-1002K11.1 | 1.52E−05 |
| Neuron | ZCCHC12 | 3.37E−04 |
| Neuron | C5orf30 | 8.16E−04 |
| Neuron | LINC00087 | 9.95E−03 |
| Pericytes | PDGFRB | 7.98E−124 |
| Pericytes | RCAN2 | 4.19E−110 |
| Pericytes | PRKG1 | 3.32E−107 |
| Pericytes | RGS6 | 1.30E−103 |
| Pericytes | KCNAB1 | 3.70E−91 |
| Pericytes | FHL5 | 3.60E−77 |
| Pericytes | ACTA2 | 5.05E−76 |
| Pericytes | NR2F2-AS1 | 4.89E−69 |
| Pericytes | HEYL | 3.77E−68 |
| Pericytes | SORBS2 | 2.07E−63 |
| Pericytes | MT2A | 5.06E−62 |
| Pericytes | DGKG | 2.64E−59 |
| Pericytes | CLMN | 3.95E−58 |
| Pericytes | MT1E | 8.22E−57 |
| Pericytes | RP11-140I24.1 | 5.67E−53 |
| Pericytes | DLC1 | 4.10E−51 |
| Pericytes | LPP | 1.06E−49 |
| Pericytes | IGFBP7 | 1.24E−46 |
| Pericytes | NTRK3 | 1.62E−46 |
| Pericytes | EDNRA | 4.11E−46 |
| Pericytes | INPP4B | 9.56E−46 |
| Pericytes | FRY | 1.69E−42 |
| Pericytes | RASAL2 | 2.81E−42 |
| Pericytes | ATP10A | 5.00E−41 |
| Pericytes | MT1M | 5.00E−41 |
| Pericytes | HES4 | 5.00E−41 |
| Pericytes | ADCY3 | 1.40E−40 |
| Pericytes | PTPRG | 2.13E−40 |
| Pericytes | ATP1B3 | 2.13E−40 |
| Pericytes | RBPMS | 3.45E−40 |
| Pericytes | TINAGL1 | 9.30E−40 |
| Pericytes | CTD-2009A10.1 | 7.25E−39 |
| Pericytes | SPARCL1 | 2.35E−36 |
| Pericytes | TBX2 | 3.68E−36 |
| Pericytes | TACC1 | 3.70E−36 |
| Pericytes | AC007401.2 | 1.87E−35 |
| Pericytes | FRMD4A | 9.09E−35 |
| Pericytes | ZBTB7C | 1.05E−34 |
| Pericytes | EBF1 | 1.98E−34 |
| Pericytes | CALD1 | 8.87E−34 |
| Pericytes | SLC7A2 | 1.21E−33 |
| Pericytes | CDH6 | 3.63E−33 |
| Pericytes | NOTCH3 | 4.21E−33 |
| Pericytes | SLIT3 | 5.29E−33 |
| Pericytes | RP11-444D3.1 | 4.14E−32 |
| Pericytes | HIP1 | 7.28E−32 |
| Pericytes | ELN | 1.18E−31 |
| Pericytes | ARHGEF7 | 1.15E−30 |
| Pericytes | SYTL2 | 1.35E−30 |
| Pericytes | PPAP2B | 3.84E−30 |
| Pericytes | SLC6A1-AS1 | 2.81E−29 |
| Pericytes | EPS8 | 4.47E−29 |
| Pericytes | RNF152 | 2.28E−28 |
| Pericytes | LDB3 | 3.00E−28 |
| Pericytes | ZFHX3 | 4.90E−28 |
| Pericytes | PICALM | 1.07E−27 |
| Pericytes | DBR1 | 1.23E−27 |
| Pericytes | CAV2 | 1.77E−26 |
| Pericytes | LPHN3 | 2.60E−26 |
| Pericytes | ID3 | 4.11E−26 |
| Pericytes | RYR2 | 6.45E−26 |
| Pericytes | JAG1 | 6.41E−25 |
| Pericytes | ZNF331 | 1.53E−24 |
| Pericytes | BAIAP2L2 | 1.78E−24 |
| Pericytes | EDIL3 | 2.01E−24 |
| Pericytes | CRIM1 | 1.20E−23 |
| Pericytes | CPM | 1.20E−23 |
| Pericytes | MCAM | 1.73E−23 |
| Pericytes | TIMP3 | 2.18E−23 |
| Pericytes | NPNT | 3.20E−23 |
| Pericytes | RP11-223C24.1 | 7.45E−23 |
| Pericytes | SLC38A11 | 7.78E−23 |
| Pericytes | ITGA8 | 1.32E−22 |
| Pericytes | PPFIA2 | 2.85E−22 |
| Pericytes | CRISPLD2 | 4.11E−22 |
| Pericytes | PTEN | 5.56E−22 |
| Pericytes | ADIRF | 1.64E−21 |
| Pericytes | SMIM12 | 2.95E−21 |
| Pericytes | STEAP4 | 3.02E−21 |
| Pericytes | GRK5 | 3.02E−21 |
| Pericytes | AC140912.1 | 6.03E−21 |
| Pericytes | FKBP5 | 6.03E−21 |
| Pericytes | NTN4 | 9.43E−21 |
| Pericytes | AC002066.1 | 1.53E−20 |
| Pericytes | CTGF | 1.69E−20 |
| Pericytes | RP11-436F23.1 | 3.25E−20 |
| Pericytes | MYO1D | 4.88E−20 |
| Pericytes | FCHSD2 | 1.80E−19 |
| Pericytes | MLIP | 3.12E−19 |
| Pericytes | RP11-315E17.1 | 7.47E−19 |
| Pericytes | HIPK2 | 8.07E−19 |
| Pericytes | RP11-1000B6.3 | 8.07E−19 |
| Pericytes | ARHGEF10L | 1.49E−18 |
| Pericytes | RBMS3 | 3.09E−18 |
| Pericytes | AGPS | 3.57E−18 |
| Pericytes | LINC01088 | 4.52E−18 |
| Pericytes | LMOD1 | 4.93E−18 |
| Pericytes | ESYT2 | 9.13E−18 |
| Pericytes | RBMS3-AS3 | 1.13E−17 |
| Pericytes | MFGE8 | 1.26E−17 |
| Pericytes | PRMT10 | 1.26E−17 |
| Pericytes | CSDC2 | 2.41E−17 |
| Pericytes | EBF2 | 7.04E−17 |
| Pericytes | PTP4A3 | 8.09E−17 |
| Pericytes | LGI4 | 1.09E−16 |
| Pericytes | CHSY3 | 1.64E−16 |
| Pericytes | AC097724.3 | 3.69E−16 |
| Pericytes | RP11-156K13.1 | 4.65E−16 |
| Pericytes | NTRK2 | 6.81E−16 |
| Pericytes | FRY-AS1 | 1.07E−15 |
| Pericytes | MYO1B | 1.65E−14 |
| Pericytes | PRDM16 | 2.66E−14 |
| Pericytes | MARK1 | 3.38E−14 |
| Pericytes | ZFAND5 | 4.16E−14 |
| Pericytes | MTHFD2 | 1.28E−13 |
| Pericytes | CPE | 3.06E−13 |
| Pericytes | ALAD | 7.01E−13 |
| Pericytes | ISYNA1 | 9.48E−13 |
| Pericytes | SLC22A3 | 4.13E−12 |
| Pericytes | WTIP | 7.11E−12 |
| Pericytes | C1QTNF1 | 1.02E−11 |
| Pericytes | LBH | 2.19E−11 |
| Pericytes | CENPO | 2.34E−11 |
| Pericytes | PTGIR | 2.66E−11 |
| Pericytes | PPP1CB | 3.02E−11 |
| Pericytes | USP2 | 1.53E−10 |
| Pericytes | LINC00989 | 2.58E−10 |
| Pericytes | SCN3A | 3.77E−10 |
| Pericytes | NR4A3 | 3.67E−09 |
| Pericytes | GPRC5C | 3.71E−09 |
| Pericytes | HEY2 | 6.68E−09 |
| Pericytes | LINC00702 | 8.26E−09 |
| Pericytes | ADRA1A | 1.06E−08 |
| Pericytes | RP11-326A19.4 | 2.40E−08 |
| Pericytes | NR2F2 | 2.44E−08 |
| Pericytes | GLDN | 1.02E−07 |
| Pericytes | MICAL1 | 1.05E−07 |
| Pericytes | HMGCLL1 | 2.19E−07 |
| Pericytes | RP5-968D22.1 | 5.71E−07 |
| Pericytes | RANBP3L | 1.15E−06 |
| Pericytes | HES1 | 1.16E−06 |
| Pericytes | ID4 | 1.28E−06 |
| Pericytes | GADD45G | 3.18E−06 |
| Pericytes | ADAMTS15 | 3.76E−06 |
| Pericytes | GPR176 | 5.23E−06 |
| Pericytes | PRRX1 | 5.69E−06 |
| Pericytes | ADAMTS4 | 1.15E−05 |
| Pericytes | FRK | 1.26E−05 |
| Pericytes | AC005863.2 | 8.91E−05 |
| Pericytes | MIR22HG | 1.27E−04 |
| SDS+ | EPHA7 | 4.53E−69 |
| SDS+ | DCC | 2.69E−48 |
| SDS+ | LSAMP-AS1 | 1.50E−44 |
| SDS+ | SDS | 2.28E−42 |
| SDS+ | AKAP12 | 3.28E−41 |
| SDS+ | SVIL | 2.33E−34 |
| SDS+ | FBXO32 | 2.63E−31 |
| SDS+ | PRUNE2 | 1.09E−28 |
| SDS+ | CTD-3105H18.18 | 4.61E−28 |
| SDS+ | DLG5 | 5.80E−28 |
| SDS+ | PDE9A | 1.37E−26 |
| SDS+ | MT1E | 1.42E−26 |
| SDS+ | RP11-707P20.1 | 1.73E−26 |
| SDS+ | SPESP1 | 2.88E−25 |
| SDS+ | RP11-690J15.1 | 4.70E−25 |
| SDS+ | GOLPH3 | 1.48E−24 |
| SDS+ | RP11-680F20.9 | 1.54E−22 |
| SDS+ | CRISPLD2 | 2.02E−22 |
| SDS+ | CACNA1C | 3.69E−22 |
| SDS+ | FSHR | 2.16E−21 |
| SDS+ | WWTR1 | 3.03E−21 |
| SDS+ | MYH14 | 3.29E−21 |
| SDS+ | RAD51 | 8.54E−21 |
| SDS+ | NID1 | 1.04E−20 |
| SDS+ | GEM | 6.36E−19 |
| SDS+ | MON1B | 6.62E−19 |
| SDS+ | CHD8 | 1.54E−18 |
| SDS+ | SDR42E2 | 1.70E−18 |
| SDS+ | RP11-296K13.4 | 2.11E−18 |
| SDS+ | RP11-169E6.4 | 2.12E−18 |
| SDS+ | LDLRAD2 | 2.24E−18 |
| SDS+ | PACRG | 3.65E−18 |
| SDS+ | PDZRN3 | 3.94E−18 |
| SDS+ | VSNL1 | 4.68E−18 |
| SDS+ | MT1X | 1.31E−17 |
| SDS+ | AFMID | 1.92E−17 |
| SDS+ | C9orf171 | 2.91E−17 |
| SDS+ | SCAI | 4.48E−17 |
| SDS+ | C15orf52 | 6.15E−17 |
| SDS+ | CASC5 | 9.51E−17 |
| SDS+ | RBM20 | 1.74E−16 |
| SDS+ | SOGA2 | 1.79E−16 |
| SDS+ | MT1M | 1.43E−15 |
| SDS+ | BMPR1A | 1.59E−15 |
| SDS+ | LINC00276 | 2.35E−15 |
| SDS+ | STK24 | 3.73E−15 |
| SDS+ | PRSS38 | 4.53E−15 |
| SDS+ | PSMA8 | 5.74E−15 |
| SDS+ | GRM7 | 6.92E−15 |
| SDS+ | RP11-432B6.3 | 9.06E−15 |
| SDS+ | C1orf95 | 5.61E−14 |
| SDS+ | SLC8A1-AS1 | 6.25E−14 |
| SDS+ | RP11-138I17.1 | 7.01E−14 |
| SDS+ | GREM2 | 8.64E−14 |
| SDS+ | ARMC2 | 1.15E−13 |
| SDS+ | CTC-529L17.2 | 1.34E−13 |
| SDS+ | CLCN4 | 1.87E−13 |
| SDS+ | CWC25 | 2.02E−13 |
| SDS+ | RP11-774D14.1 | 2.31E−13 |
| SDS+ | RPGRIP1 | 3.03E−13 |
| SDS+ | MITF | 3.65E−13 |
| SDS+ | ATP6V0A4 | 3.70E−13 |
| SDS+ | FMN2 | 4.88E−13 |
| SDS+ | GTF2IRD1 | 4.92E−13 |
| SDS+ | GPC3 | 6.44E−13 |
| SDS+ | CDHR3 | 8.00E−13 |
| SDS+ | UNC79 | 1.07E−12 |
| SDS+ | GPN3 | 1.07E−12 |
| SDS+ | DPP6 | 1.10E−12 |
| SDS+ | SGK223 | 1.18E−12 |
| SDS+ | SLC24A3 | 1.26E−12 |
| SDS+ | RBFOX3 | 1.33E−12 |
| SDS+ | TTTY14 | 1.53E−12 |
| SDS+ | RP5-1048B16.1 | 1.83E−12 |
| SDS+ | FAM153B | 2.16E−12 |
| SDS+ | SORBS2 | 2.22E−12 |
| SDS+ | CDC40 | 4.57E−12 |
| SDS+ | AC004076.9 | 5.17E−12 |
| SDS+ | AFF3 | 5.90E−12 |
| SDS+ | RP1-209A6.1 | 7.41E−12 |
| SDS+ | ANP32E | 8.02E−12 |
| SDS+ | ACBD5 | 8.26E−12 |
| SDS+ | FGFR2 | 1.41E−11 |
| SDS+ | SMO | 1.75E−11 |
| SDS+ | MAMDC2-AS1 | 2.02E−11 |
| SDS+ | ROR2 | 2.83E−11 |
| SDS+ | CCBE1 | 4.29E−11 |
| SDS+ | RP11-545G3.1 | 4.43E−11 |
| SDS+ | SYNPO | 4.58E−11 |
| SDS+ | AC007389.3 | 6.25E−11 |
| SDS+ | EPB41 | 7.17E−11 |
| SDS+ | COL4A3 | 7.74E−11 |
| SDS+ | TIGD7 | 7.82E−11 |
| SDS+ | DENND5B-AS1 | 8.69E−11 |
| SDS+ | MED22 | 1.82E−10 |
| SDS+ | COL23A1 | 2.61E−10 |
| SDS+ | RIMS2 | 2.94E−10 |
| SDS+ | ESRRG | 4.13E−10 |
| SDS+ | METTL2B | 4.50E−10 |
| SDS+ | CCDC62 | 5.26E−10 |
| SDS+ | LY9 | 1.13E−09 |
| SDS+ | PPAPDC1A | 3.03E−09 |
| SDS+ | DYRK3 | 5.08E−09 |
| SDS+ | RAB5B | 8.98E−09 |
| SDS+ | AC073635.5 | 1.31E−08 |
| SDS+ | KIAA1644 | 3.16E−08 |
| SDS+ | RP11-818F20.5 | 4.46E−08 |
| SDS+ | AL022476.2 | 6.58E−08 |
| SDS+ | HTRA4 | 1.27E−07 |
| SDS+ | FAM25C | 1.83E−07 |
| SDS+ | RP3-404K8.2 | 2.78E−07 |
| SDS+ | RP11-195F19.9 | 4.09E−07 |
| SDS+ | RP11-555J4.4 | 4.90E−07 |
| SDS+ | MT1G | 6.17E−07 |
| SDS+ | RP5-884C9.2 | 6.96E−07 |
| SDS+ | RASGEF1C | 7.68E−07 |
| SDS+ | C1orf87 | 1.05E−06 |
| SDS+ | VIL1 | 1.18E−06 |
| SDS+ | DBNDD1 | 1.66E−06 |
| SDS+ | SLC34A1 | 2.40E−06 |
| SDS+ | CABYR | 4.05E−06 |
| SDS+ | GPR133 | 4.21E−06 |
| SDS+ | ALDH3B1 | 4.21E−06 |
| SDS+ | RP11-46I8.3 | 4.43E−06 |
| SDS+ | TUSC5 | 1.35E−05 |
| SDS+ | RP11-297A16.2 | 2.03E−05 |
| SDS+ | RP11-24I21.1 | 2.50E−05 |
| SDS+ | MASP1 | 2.82E−05 |
| SDS+ | CTD-2152M20.2 | 2.89E−05 |
| SDS+ | KCNJ12 | 3.40E−05 |
| SDS+ | SLC1A5 | 3.71E−05 |
| SDS+ | NPFFR1 | 3.74E−05 |
| SDS+ | SPATA12 | 4.36E−05 |
| SDS+ | GRTP1-AS1 | 5.47E−05 |
| SDS+ | ADAM18 | 5.50E−05 |
| SDS+ | GINS2 | 6.75E−05 |
| SDS+ | RP11-32F11.2 | 1.16E−04 |
| SDS+ | DSCR9 | 1.50E−04 |
| SDS+ | KNDC1 | 1.54E−04 |
| SDS+ | NKD1 | 1.64E−04 |
| SDS+ | CTD-2231H16.1 | 1.78E−04 |
| SDS+ | ARSH | 2.56E−04 |
| SDS+ | CCL24 | 3.56E−04 |
| SDS+ | GAD2 | 3.89E−04 |
| SDS+ | RP11-46H11.3 | 5.42E−04 |
| SDS+ | RP11-167N24.4 | 5.56E−04 |
| SDS+ | RP11-83M16.6 | 6.42E−04 |
| SDS+ | KL | 6.59E−04 |
| SDS+ | RP11-483H20.6 | 7.57E−04 |
| SDS+ | TPH2 | 7.60E−04 |
| SDS+ | CTD-2251F13.1 | 1.19E−03 |
| SDS+ | LDHAL6A | 1.46E−03 |
| SDS+ | AL161784.1 | 1.71E−03 |
| SDS+ | RP11-133L19.1 | 1.76E−03 |
| SDS+ | RP11-108P20.4 | 2.32E−03 |
| SDS+ | DSC3 | 2.45E−03 |
| SDS+ | ZNF574 | 2.80E−03 |
| SDS+ | C1orf100 | 2.83E−03 |
| SDS+ | FAM189A2 | 3.59E−03 |
| SDS+ | TRIM67 | 4.53E−03 |
| SDS+ | MICALCL | 6.29E−03 |
| SDS+ | KDM5D | 7.36E−03 |
| SDS+ | HTR3A | 9.13E−03 |
| SDS+ | CTD-2377D24.8 | 9.60E−03 |
| SDS+ | VSTM1 | 1.22E−02 |
| SDS+ | CTB-5E10.3 | 1.62E−02 |
| SDS+ | ADRA1B | 1.66E−02 |
| SDS+ | RP11-3D4.2 | 1.82E−02 |
| SDS+ | IQGAP3 | 2.10E−02 |
| SPP1+ | MT-CO3 | 1.01E−141 |
| SPP1+ | MT-CO1 | 4.52E−131 |
| SPP1+ | MT-CO2 | 1.02E−114 |
| SPP1+ | MT-ND3 | 4.28E−114 |
| SPP1+ | MT-ND4 | 8.43E−109 |
| SPP1+ | MT-ATP6 | 6.69E−107 |
| SPP1+ | MT-CYB | 2.19E−105 |
| SPP1+ | MT-ND1 | 2.42E−95 |
| SPP1+ | MT-ND2 | 9.46E−93 |
| SPP1+ | MT-ND5 | 7.78E−73 |
| SPP1+ | MTRNR2L8 | 5.87E−40 |
| SPP1+ | SPP1 | 2.68E−36 |
| SPP1+ | MT-ND4L | 1.26E−31 |
| SPP1+ | DHFR | 1.91E−30 |
| SPP1+ | MTRNR2L10 | 6.66E−28 |
| SPP1+ | DEFB1 | 1.19E−23 |
| SPP1+ | ATP1B1 | 8.67E−22 |
| SPP1+ | ITM2B | 8.23E−20 |
| SPP1+ | MTRNR2L12 | 1.04E−19 |
| SPP1+ | MT-ND6 | 8.65E−19 |
| SPP1+ | EGF | 1.64E−17 |
| SPP1+ | KCNJ16 | 5.18E−17 |
| SPP1+ | ESRRG | 7.29E−17 |
| SPP1+ | PKHD1 | 3.14E−16 |
| SPP1+ | ERBB4 | 6.28E−16 |
| SPP1+ | KNG1 | 3.73E−15 |
| SPP1+ | SLC12A3 | 3.73E−15 |
| SPP1+ | SLC12A1 | 1.01E−14 |
| SPP1+ | AC013463.2 | 3.32E−14 |
| SPP1+ | UMOD | 1.21E−12 |
| SPP1+ | CDH16 | 1.36E−12 |
| SPP1+ | PTH1R | 4.26E−12 |
| SPP1+ | ATP1A1 | 1.48E−10 |
| SPP1+ | AC073218.2 | 2.79E−10 |
| SPP1+ | HINT1 | 5.64E−10 |
| SPP1+ | PAX8 | 6.35E−10 |
| SPP1+ | CCSER1 | 1.91E−09 |
| SPP1+ | TPT1 | 2.05E−09 |
| SPP1+ | MT1G | 2.32E−09 |
| SPP1+ | CA12 | 2.61E−09 |
| SPP1+ | CYB5A | 3.66E−09 |
| SPP1+ | COX7B | 5.78E−09 |
| SPP1+ | HSD11B2 | 1.57E−08 |
| SPP1+ | TMBIM6 | 1.72E−08 |
| SPP1+ | NDUFA4 | 2.90E−08 |
| SPP1+ | RP5-857K21.4 | 4.77E−08 |
| SPP1+ | SLC16A12 | 4.77E−08 |
| SPP1+ | PEBP1 | 5.98E−08 |
| SPP1+ | CGNL1 | 6.25E−08 |
| SPP1+ | RPL34 | 6.25E−08 |
| SPP1+ | COX6C | 1.14E−07 |
| SPP1+ | TFCP2L1 | 1.38E−07 |
| SPP1+ | COX7C | 1.76E−07 |
| SPP1+ | SKP1 | 1.78E−07 |
| SPP1+ | OGDHL | 2.42E−07 |
| SPP1+ | ATP6V1F | 5.05E−07 |
| SPP1+ | MTRNR2L1 | 5.20E−07 |
| SPP1+ | PTH2R | 7.50E−07 |
| SPP1+ | RPS23 | 7.50E−07 |
| SPP1+ | TTTY14 | 8.13E−07 |
| SPP1+ | ISCU | 1.33E−06 |
| SPP1+ | TMEM52B | 1.58E−06 |
| SPP1+ | GPC5 | 1.65E−06 |
| SPP1+ | RPL7 | 1.72E−06 |
| SPP1+ | MECOM | 2.63E−06 |
| SPP1+ | FTH1 | 2.71E−06 |
| SPP1+ | IVNS1ABP | 2.73E−06 |
| SPP1+ | PCK1 | 3.71E−06 |
| SPP1+ | COBLL1 | 3.71E−06 |
| SPP1+ | RPS27A | 4.44E−06 |
| SPP1+ | KDM5B | 5.77E−06 |
| SPP1+ | OOEP | 5.86E−06 |
| SPP1+ | LAMTOR5 | 5.94E−06 |
| SPP1+ | FTL | 6.49E−06 |
| SPP1+ | HSP90AB1 | 7.25E−06 |
| SPP1+ | ATP6V1G1 | 7.74E−06 |
| SPP1+ | AHCYL1 | 8.31E−06 |
| SPP1+ | SNX10 | 8.75E−06 |
| SPP1+ | KIF12 | 8.75E−06 |
| SPP1+ | GPX3 | 9.33E−06 |
| SPP1+ | AC002539.1 | 1.25E−05 |
| SPP1+ | ALDOB | 1.29E−05 |
| SPP1+ | RP1-60O19.1 | 1.31E−05 |
| SPP1+ | HSPD1 | 1.31E−05 |
| SPP1+ | CD164 | 1.39E−05 |
| SPP1+ | MTRNR2L3 | 1.41E−05 |
| SPP1+ | PLCL1 | 1.57E−05 |
| SPP1+ | COX5B | 1.59E−05 |
| SPP1+ | C14orf105 | 1.60E−05 |
| SPP1+ | NGFRAP1 | 1.60E−05 |
| SPP1+ | S100A10 | 1.67E−05 |
| SPP1+ | DBI | 1.68E−05 |
| SPP1+ | OXR1 | 1.93E−05 |
| SPP1+ | MT1F | 1.98E−05 |
| SPP1+ | DUSP9 | 1.98E−05 |
| SPP1+ | TXNIP | 2.10E−05 |
| SPP1+ | MPC1 | 2.24E−05 |
| SPP1+ | ATP6V0E1 | 3.13E−05 |
| SPP1+ | SOD1 | 3.13E−05 |
| SPP1+ | UGT2B7 | 3.16E−05 |
| SPP1+ | RP4-655J12.4 | 8.05E−05 |
| SPP1+ | WRNIP1 | 1.09E−04 |
| SPP1+ | SFRP1 | 1.20E−04 |
| SPP1+ | KL | 1.60E−04 |
| SPP1+ | SLC6A8 | 1.64E−04 |
| SPP1+ | GATM | 2.31E−04 |
| SPP1+ | RP11-465B22.8 | 3.18E−04 |
| SPP1+ | GADD45A | 3.22E−04 |
| SPP1+ | KLHDC7A | 3.70E−04 |
| SPP1+ | PPP1R1A | 3.77E−04 |
| SPP1+ | FXYD2 | 3.78E−04 |
| SPP1+ | PDZK1IP1 | 4.20E−04 |
| SPP1+ | TMEM101 | 6.06E−04 |
| SPP1+ | CA2 | 6.25E−04 |
| SPP1+ | TFAP2A | 8.34E−04 |
| SPP1+ | PCSK1N | 8.70E−04 |
| SPP1+ | SFTPD | 1.29E−03 |
| SPP1+ | SIM1 | 1.38E−03 |
| SPP1+ | CLDN8 | 1.40E−03 |
| SPP1+ | TMEM72 | 1.52E−03 |
| SPP1+ | SMIM5 | 1.61E−03 |
| SPP1+ | SHISA3 | 1.64E−03 |
| SPP1+ | TRIM50 | 2.01E−03 |
| SPP1+ | MT1H | 2.24E−03 |
| SPP1+ | ACSM2B | 2.80E−03 |
| SPP1+ | TMEM27 | 2.99E−03 |
| SPP1+ | PROM2 | 3.02E−03 |
| SPP1+ | CYP4A11 | 3.85E−03 |
| SPP1+ | GDF15 | 4.14E−03 |
| SPP1+ | TNFRSF11B | 4.28E−03 |
| SPP1+ | S100A2 | 4.39E−03 |
| SPP1+ | MTRNR2L6 | 4.86E−03 |
| SPP1+ | TSR3 | 5.82E−03 |
| SPP1+ | CYP4F3 | 6.14E−03 |
| SPP1+ | KRT18 | 6.60E−03 |
| SPP1+ | CLCNKA | 7.03E−03 |
| SPP1+ | COMMD8 | 9.88E−03 |
| SPP1+ | BEX2 | 1.12E−02 |
| SPP1+ | NAT8 | 1.21E−02 |
| SPP1+ | RP11-513O17.2 | 1.26E−02 |
| SPP1+ | TIMM21 | 1.29E−02 |
| SPP1+ | ARHGEF16 | 1.30E−02 |
| SPP1+ | GSTO2 | 1.46E−02 |
| SPP1+ | LINC00958 | 1.64E−02 |
| SPP1+ | MFAP3L | 2.17E−02 |
| SPP1+ | TM7SF2 | 2.18E−02 |
| SPP1+ | GSTA1 | 2.25E−02 |
| SPP1+ | SLC37A4 | 2.85E−02 |
| SPP1+ | TM2D2 | 3.21E−02 |
| SPP1+ | TMEM37 | 3.31E−02 |
| SPP1+ | RBM15B | 3.65E−02 |
| SPP1+ | EGOT | 4.10E−02 |
| SPP1+ | PPP1R26 | 4.53E−02 |
| T cells | ARHGAP15 | 2.09E−129 |
| T cells | PTPRC | 1.44E−101 |
| T cells | ANKRD44 | 3.72E−93 |
| T cells | FAM65B | 3.69E−77 |
| T cells | CXCR4 | 2.40E−62 |
| T cells | SKAP1 | 1.67E−59 |
| T cells | CELF2 | 1.84E−58 |
| T cells | CCND3 | 3.16E−58 |
| T cells | IL7R | 1.21E−54 |
| T cells | RCSD1 | 5.76E−54 |
| T cells | BTG1 | 1.82E−53 |
| T cells | THEMIS | 2.28E−53 |
| T cells | ETS1 | 4.24E−51 |
| T cells | BCL2 | 1.72E−49 |
| T cells | PRKCB | 4.15E−49 |
| T cells | TNFAIP8 | 8.39E−49 |
| T cells | RHOH | 9.01E−49 |
| T cells | RP11-347P5.1 | 9.06E−49 |
| T cells | TXK | 9.66E−49 |
| T cells | TC2N | 6.60E−48 |
| T cells | MAML2 | 3.56E−47 |
| T cells | TMC8 | 4.25E−46 |
| T cells | CD96 | 1.67E−45 |
| T cells | RP11-277P12.20 | 8.80E−42 |
| T cells | STK17B | 2.83E−41 |
| T cells | BCL11B | 6.26E−41 |
| T cells | CDC42SE2 | 1.92E−39 |
| T cells | IKZF1 | 4.34E−39 |
| T cells | LEF1 | 1.46E−38 |
| T cells | SSH2 | 3.05E−38 |
| T cells | PARP8 | 1.15E−37 |
| T cells | PDE7A | 1.84E−37 |
| T cells | SCML4 | 4.08E−37 |
| T cells | CHST11 | 4.96E−37 |
| T cells | KIAA0922 | 4.98E−37 |
| T cells | BACH 2 | 7.99E−37 |
| T cells | CAMK4 | 3.40E−36 |
| T cells | DOCK10 | 5.44E−36 |
| T cells | PIP4K2A | 1.79E−35 |
| T cells | CD69 | 2.39E−35 |
| T cells | RABGAP1L | 8.25E−35 |
| T cells | MS4A1 | 1.28E−34 |
| T cells | CD247 | 1.51E−33 |
| T cells | FYN | 2.33E−33 |
| T cells | FYB | 4.23E−33 |
| T cells | RP11-553K8.5 | 4.30E−33 |
| T cells | DOCK8 | 4.61E−33 |
| T cells | STK4 | 8.42E−33 |
| T cells | CLEC2D | 2.52E−32 |
| T cells | CD53 | 5.48E−32 |
| T cells | ITK | 1.06E−31 |
| T cells | ATM | 2.05E−31 |
| T cells | AC104820.2 | 2.95E−31 |
| T cells | EVL | 2.95E−31 |
| T cells | LINC00861 | 6.89E−31 |
| T cells | STAT4 | 1.71E−30 |
| T cells | SEMA4D | 3.70E−30 |
| T cells | PRKCH | 3.85E−30 |
| T cells | TRAF3IP3 | 7.91E−30 |
| T cells | PRKCQ | 1.16E−29 |
| T cells | HLA-B | 5.20E−29 |
| T cells | INPP4B | 1.52E−28 |
| T cells | PACS1 | 3.30E−27 |
| T cells | CTB-4E7.1 | 9.07E−27 |
| T cells | HLA-C | 3.32E−26 |
| T cells | SYTL3 | 3.55E−26 |
| T cells | KLF12 | 6.32E−26 |
| T cells | MBNL1 | 6.32E−26 |
| T cells | B2M | 8.86E−26 |
| T cells | HLA-A | 1.17E−25 |
| T cells | PIK3IP1 | 1.26E−25 |
| T cells | CARD11 | 3.54E−25 |
| T cells | TXNIP | 5.02E−25 |
| T cells | EMB | 1.97E−24 |
| T cells | BANK1 | 2.48E−24 |
| T cells | SH3KBP1 | 2.54E−24 |
| T cells | RUNX3 | 3.83E−24 |
| T cells | PRKCQ-AS1 | 4.78E−24 |
| T cells | APBB1IP | 7.71E−24 |
| T cells | GRAP2 | 1.02E−23 |
| T cells | RASA2 | 5.99E−23 |
| T cells | ITGA4 | 1.51E−22 |
| T cells | SMCHD1 | 2.02E−22 |
| T cells | CYTIP | 2.03E−22 |
| T cells | CD3D | 2.91E−21 |
| T cells | ANK3 | 3.03E−21 |
| T cells | RUNX1 | 9.53E−21 |
| T cells | PITPNC1 | 9.66E−21 |
| T cells | SERINC5 | 1.17E−20 |
| T cells | ADAM28 | 1.30E−20 |
| T cells | PYHIN1 | 2.21E−20 |
| T cells | MGAT5 | 2.58E−20 |
| T cells | CD3G | 1.08E−19 |
| T cells | FAIM3 | 1.28E−19 |
| T cells | STK17A | 1.28E−19 |
| T cells | IQGAP2 | 1.34E−19 |
| T cells | RP5-1022J11.2 | 2.07E−19 |
| T cells | SLFN12L | 1.02E−18 |
| T cells | RP11-624C23.1 | 1.02E−18 |
| T cells | OXNAD1 | 1.33E−18 |
| T cells | SLA | 1.83E−18 |
| T cells | CD37 | 4.59E−17 |
| T cells | SIDT1 | 1.06E−16 |
| T cells | AIM1 | 1.14E−16 |
| T cells | ACAP1 | 1.29E−16 |
| T cells | TBC1D10C | 3.73E−16 |
| T cells | ZAP70 | 1.00E−15 |
| T cells | SELL | 1.07E−15 |
| T cells | ICOS | 1.32E−15 |
| T cells | ITGB2-AS1 | 4.30E−15 |
| T cells | AMICA1 | 4.46E−15 |
| T cells | CCR7 | 6.30E−15 |
| T cells | LINC00926 | 1.48E−14 |
| T cells | RP11-456D7.1 | 2.53E−14 |
| T cells | ARHGAP25 | 7.45E−14 |
| T cells | LTB | 8.09E−14 |
| T cells | KLRB1 | 1.09E−13 |
| T cells | LCP1 | 1.09E−13 |
| T cells | CD2 | 1.14E−13 |
| T cells | SPOCK2 | 1.14E−13 |
| T cells | SLAMF1 | 1.96E−13 |
| T cells | TRABD2A | 2.02E−13 |
| T cells | CD52 | 7.07E−13 |
| T cells | GIMAP7 | 9.52E−13 |
| T cells | LCP2 | 1.45E−12 |
| T cells | TNFSF8 | 6.51E−12 |
| T cells | KIAA1551 | 9.31E−12 |
| T cells | AC092580.4 | 1.22E−11 |
| T cells | CD7 | 1.25E−11 |
| T cells | CD6 | 1.66E−11 |
| T cells | GPRIN3 | 4.00E−10 |
| T cells | TESPA1 | 4.49E−10 |
| T cells | MIR155HG | 9.44E−10 |
| T cells | BLK | 1.03E−09 |
| T cells | SLAMF6 | 1.97E−09 |
| T cells | MYBL1 | 3.27E−09 |
| T cells | BFSP2 | 6.81E−09 |
| T cells | FCRL1 | 7.56E−09 |
| T cells | TRAT1 | 1.65E−08 |
| T cells | IRF8 | 1.65E−08 |
| T cells | FAM196B | 4.88E−08 |
| T cells | BTN3A1 | 6.00E−08 |
| T cells | LIMD2 | 3.62E−07 |
| T cells | ISG20 | 1.83E−06 |
| T cells | CD28 | 1.88E−05 |
| VEGFC+ | LDB2 | 5.22E−80 |
| VEGFC+ | MCTP1 | 2.35E−73 |
| VEGFC+ | TCF4 | 2.66E−70 |
| VEGFC+ | ARL15 | 5.74E−70 |
| VEGFC+ | MECOM | 1.18E−69 |
| VEGFC+ | RALGAPA2 | 1.37E−62 |
| VEGFC+ | MAGI1 | 3.61E−58 |
| VEGFC+ | EPAS1 | 4.59E−58 |
| VEGFC+ | PTPRM | 1.35E−56 |
| VEGFC+ | PITPNC1 | 6.01E−56 |
| VEGFC+ | ABLIM1 | 2.50E−55 |
| VEGFC+ | MAST4 | 5.49E−53 |
| VEGFC+ | PTPRB | 1.82E−48 |
| VEGFC+ | VWF | 4.39E−43 |
| VEGFC+ | PLEKHG1 | 5.99E−42 |
| VEGFC+ | SASH1 | 2.17E−41 |
| VEGFC+ | RASGRF2 | 1.77E−40 |
| VEGFC+ | TMTC1 | 3.95E−40 |
| VEGFC+ | PREX2 | 6.37E−40 |
| VEGFC+ | MYO1E | 1.69E−39 |
| VEGFC+ | ABLIM3 | 3.55E−38 |
| VEGFC+ | ADAMTS9 | 8.00E−38 |
| VEGFC+ | TSHZ2 | 4.63E−37 |
| VEGFC+ | WWTR1 | 5.97E−37 |
| VEGFC+ | PRKCH | 1.41E−36 |
| VEGFC+ | ELTD1 | 7.76E−36 |
| VEGFC+ | TACC1 | 1.28E−35 |
| VEGFC+ | RAPGEF5 | 2.17E−35 |
| VEGFC+ | DOCK9 | 8.33E−35 |
| VEGFC+ | MEF2C | 1.39E−34 |
| VEGFC+ | VEGFC | 1.79E−34 |
| VEGFC+ | PLCB4 | 4.34E−34 |
| VEGFC+ | NEDD9 | 4.46E−34 |
| VEGFC+ | NRP1 | 1.87E−33 |
| VEGFC+ | SEC14L1 | 2.05E−31 |
| VEGFC+ | EGFL7 | 3.63E−29 |
| VEGFC+ | EVA1C | 7.87E−29 |
| VEGFC+ | ST6GALNAC3 | 8.85E−29 |
| VEGFC+ | TPO | 1.52E−28 |
| VEGFC+ | RAPGEF1 | 4.98E−28 |
| VEGFC+ | RP3-510L9.1 | 5.06E−27 |
| VEGFC+ | NFIB | 7.44E−27 |
| VEGFC+ | MKL2 | 8.36E−27 |
| VEGFC+ | ERG | 2.25E−26 |
| VEGFC+ | IGFBP3 | 1.51E−25 |
| VEGFC+ | FLT1 | 1.74E−25 |
| VEGFC+ | CLDN5 | 3.53E−25 |
| VEGFC+ | ENPP2 | 9.08E−25 |
| VEGFC+ | SPRY1 | 2.02E−24 |
| VEGFC+ | ELMO1 | 3.55E−24 |
| VEGFC+ | XAF1 | 3.67E−24 |
| VEGFC+ | PKP4 | 3.72E−24 |
| VEGFC+ | UTRN | 9.96E−24 |
| VEGFC+ | CD93 | 1.17E−23 |
| VEGFC+ | PALMD | 1.27E−23 |
| VEGFC+ | MYH9 | 8.97E−23 |
| VEGFC+ | EMCN | 1.09E−22 |
| VEGFC+ | AQP1 | 5.49E−22 |
| VEGFC+ | PPAP2A | 1.45E−21 |
| VEGFC+ | ZNF385D | 1.80E−21 |
| VEGFC+ | GNAQ | 2.55E−21 |
| VEGFC+ | PPP1R16B | 2.66E−21 |
| VEGFC+ | GALNT18 | 4.43E−21 |
| VEGFC+ | ANO2 | 1.92E−20 |
| VEGFC+ | CRIM1 | 3.32E−20 |
| VEGFC+ | CYYR1 | 6.44E−20 |
| VEGFC+ | CTTNBP2NL | 6.64E−20 |
| VEGFC+ | GRB10 | 9.31E−20 |
| VEGFC+ | NDRG1 | 1.04E−19 |
| VEGFC+ | SYNE2 | 1.49E−19 |
| VEGFC+ | MTUS1 | 1.54E−19 |
| VEGFC+ | TGFBR2 | 2.16E−19 |
| VEGFC+ | IFI44L | 2.77E−19 |
| VEGFC+ | PICALM | 3.39E−19 |
| VEGFC+ | FLI1 | 9.86E−19 |
| VEGFC+ | SPARCL1 | 1.74E−18 |
| VEGFC+ | THSD7A | 2.07E−18 |
| VEGFC+ | RAPGEF4 | 2.11E−18 |
| VEGFC+ | MPZL2 | 2.22E−18 |
| VEGFC+ | TM4SF1 | 3.08E−18 |
| VEGFC+ | DOCK4 | 4.86E−18 |
| VEGFC+ | CADPS2 | 5.68E−18 |
| VEGFC+ | DACH1 | 1.64E−17 |
| VEGFC+ | RBMS2 | 1.75E−17 |
| VEGFC+ | PDLIM1 | 2.31E−17 |
| VEGFC+ | ARHGAP29 | 2.51E−17 |
| VEGFC+ | IFI44 | 4.32E−17 |
| VEGFC+ | LIMCH1 | 5.34E−17 |
| VEGFC+ | EXOC6 | 9.62E−17 |
| VEGFC+ | SWAP70 | 9.84E−17 |
| VEGFC+ | ARHGAP31 | 1.01E−16 |
| VEGFC+ | CCNY | 1.48E−16 |
| VEGFC+ | PPP3CA | 1.66E−16 |
| VEGFC+ | PIK3R3 | 3.80E−16 |
| VEGFC+ | EPB41L4A | 4.94E−16 |
| VEGFC+ | AL035610.2 | 5.86E−16 |
| VEGFC+ | PDE10A | 1.13E−15 |
| VEGFC+ | DOCK1 | 1.26E−15 |
| VEGFC+ | PODXL | 1.74E−15 |
| VEGFC+ | CXorf36 | 1.77E−15 |
| VEGFC+ | NEURL1B | 2.06E−15 |
| VEGFC+ | RP11-435O5.2 | 3.51E−15 |
| VEGFC+ | SLC9C1 | 4.30E−15 |
| VEGFC+ | ACER2 | 5.38E−15 |
| VEGFC+ | C10orf10 | 1.39E−14 |
| VEGFC+ | AJ239322.3 | 1.45E−14 |
| VEGFC+ | ICAM2 | 4.01E−14 |
| VEGFC+ | BHLHE40 | 8.69E−14 |
| VEGFC+ | STC1 | 2.11E−13 |
| VEGFC+ | FKBP1A | 2.74E−13 |
| VEGFC+ | CD59 | 5.51E−13 |
| VEGFC+ | SIPA1L2 | 8.59E−13 |
| VEGFC+ | RAMP3 | 3.57E−12 |
| VEGFC+ | CTC-484P3.3 | 1.12E−11 |
| VEGFC+ | RP11-834C11.3 | 2.62E−11 |
| VEGFC+ | SLC45A4 | 5.33E−11 |
| VEGFC+ | AC011526.1 | 6.19E−10 |
| VEGFC+ | SIK1 | 6.49E−10 |
| VEGFC+ | MEOX2 | 1.05E−09 |
| VEGFC+ | TEK | 2.21E−09 |
| VEGFC+ | DLL4 | 2.38E−09 |
| VEGFC+ | NOTCH4 | 2.65E−09 |
| VEGFC+ | RP1-55C23.7 | 6.62E−09 |
| VEGFC+ | ADCY4 | 7.50E−09 |
| VEGFC+ | CLEC14A | 1.05E−08 |
| VEGFC+ | AC010084.1 | 1.40E−08 |
| VEGFC+ | MMRN2 | 1.63E−08 |
| VEGFC+ | JAG1 | 4.71E−08 |
| VEGFC+ | S1PR1 | 2.43E−07 |
| VEGFC+ | BTNL9 | 2.43E−07 |
| VEGFC+ | SHANK3 | 2.64E−07 |
| VEGFC+ | CLEC1A | 4.90E−07 |
| VEGFC+ | LRRC32 | 5.79E−07 |
| VEGFC+ | RP11-805F19.2 | 6.57E−07 |
| VEGFC+ | CD160 | 1.20E−06 |
| VEGFC+ | TSPAN7 | 1.44E−06 |
| VEGFC+ | EFNB2 | 5.34E−06 |
| VEGFC+ | IGF2 | 6.20E−06 |
| VEGFC+ | LINC00312 | 2.15E−05 |
| VEGFC+ | RASA4B | 5.00E−05 |
| VEGFC+ | MYCT1 | 6.06E−05 |
| VEGFC+ | GJA1 | 6.47E−05 |
| VEGFC+ | PHF10 | 9.40E−05 |
| VEGFC+ | TEX22 | 1.12E−04 |
| VEGFC+ | SERPINE1 | 1.59E−04 |
| VEGFC+ | TNFAIP1 | 3.23E−04 |
| VEGFC+ | TAOK2 | 4.46E−04 |
| VEGFC+ | DUSP5 | 4.51E−04 |
| VEGFC+ | SHROOM1 | 4.62E−04 |
| VEGFC+ | LINC00968 | 5.26E−04 |
| VEGFC+ | LMCD1 | 1.02E−03 |
| VEGFC+ | THBD | 1.15E−03 |
| VEGFC+ | RP11-90K6.1 | 1.46E−03 |
| VEGFC+ | SLC10A6 | 1.63E−03 |
| VEGFC+ | RP11-420O16.1 | 2.05E−03 |
| VEGFC+ | MID2 | 3.95E−03 |
| VEGFC+ | GUCA1C | 4.09E−02 |
| Lymphatic endothelial | PKHD1L1 | 0.00E+00 |
| Lymphatic endothelial | MMRN1 | 1.65E−229 |
| Lymphatic endothelial | CCL21 | 3.71E−187 |
| Lymphatic endothelial | AC007319.1 | 1.74E−184 |
| Lymphatic endothelial | PPFIBP1 | 7.34E−184 |
| Lymphatic endothelial | CD36 | 9.13E−174 |
| Lymphatic endothelial | ST6GALNAC3 | 4.44E−156 |
| Lymphatic endothelial | RELN | 5.25E−156 |
| Lymphatic endothelial | TFPI | 2.85E−132 |
| Lymphatic endothelial | TSHZ2 | 3.65E−129 |
| Lymphatic endothelial | CTD-3179P9.1 | 1.81E−128 |
| Lymphatic endothelial | KALRN | 1.90E−124 |
| Lymphatic endothelial | RP4-678D15.1 | 3.06E−113 |
| Lymphatic endothelial | RP11-782C8.2 | 3.15E−112 |
| Lymphatic endothelial | LYVE1 | 1.18E−110 |
| Lymphatic endothelial | RHOJ | 7.76E−106 |
| Lymphatic endothelial | DOCK5 | 2.92E−105 |
| Lymphatic endothelial | EFNA5 | 1.29E−102 |
| Lymphatic endothelial | RP11-417J8.6 | 4.78E−101 |
| Lymphatic endothelial | CTB-118N6.3 | 4.68E−96 |
| Lymphatic endothelial | PTPRE | 1.93E−90 |
| Lymphatic endothelial | ARHGAP26 | 3.42E−87 |
| Lymphatic endothelial | LDB2 | 1.04E−79 |
| Lymphatic endothelial | MMP28 | 4.23E−78 |
| Lymphatic endothelial | DLG1 | 6.24E−74 |
| Lymphatic endothelial | STOX2 | 8.86E−73 |
| Lymphatic endothelial | EMP1 | 1.54E−72 |
| Lymphatic endothelial | SLC22A23 | 9.56E−72 |
| Lymphatic endothelial | CTB-107G13.1 | 2.47E−71 |
| Lymphatic endothelial | KANK3 | 6.39E−68 |
| Lymphatic endothelial | PROX1 | 2.35E−64 |
| Lymphatic endothelial | SASH1 | 3.47E−62 |
| Lymphatic endothelial | MAGI1 | 1.74E−61 |
| Lymphatic endothelial | C6orf141 | 4.73E−61 |
| Lymphatic endothelial | KIAA1671 | 6.56E−60 |
| Lymphatic endothelial | GPR97 | 2.27E−59 |
| Lymphatic endothelial | VAV3 | 1.45E−58 |
| Lymphatic endothelial | PPAP2A | 4.81E−58 |
| Lymphatic endothelial | TBX1 | 1.39E−56 |
| Lymphatic endothelial | KLF6 | 2.28E−56 |
| Lymphatic endothelial | TIMP3 | 2.30E−56 |
| Lymphatic endothelial | STON2 | 3.10E−56 |
| Lymphatic endothelial | TLL1 | 7.04E−54 |
| Lymphatic endothelial | ZDHHC14 | 1.02E−52 |
| Lymphatic endothelial | AC139100.3 | 4.29E−51 |
| Lymphatic endothelial | CNKSR3 | 1.41E−48 |
| Lymphatic endothelial | PARD6G | 2.56E−48 |
| Lymphatic endothelial | SPTBN1 | 5.73E−48 |
| Lymphatic endothelial | PIEZO2 | 1.11E−47 |
| Lymphatic endothelial | SNTG2 | 4.94E−46 |
| Lymphatic endothelial | NHSL1 | 1.09E−45 |
| Lymphatic endothelial | FRMD4B | 1.54E−45 |
| Lymphatic endothelial | RALGAPA2 | 3.16E−44 |
| Lymphatic endothelial | PIK3C2G | 1.77E−43 |
| Lymphatic endothelial | NRG3 | 7.59E−42 |
| Lymphatic endothelial | LRRC1 | 8.12E−42 |
| Lymphatic endothelial | CLDN5 | 1.00E−41 |
| Lymphatic endothelial | TFF3 | 1.07E−41 |
| Lymphatic endothelial | GRAPL | 1.36E−40 |
| Lymphatic endothelial | SEMA6A | 2.52E−40 |
| Lymphatic endothelial | ARHGAP29 | 9.09E−40 |
| Lymphatic endothelial | PDE1A | 3.70E−39 |
| Lymphatic endothelial | LRCOL1 | 6.69E−39 |
| Lymphatic endothelial | NR2F2-AS1 | 2.26E−37 |
| Lymphatic endothelial | SAP30BP | 9.46E−37 |
| Lymphatic endothelial | RP11-435B5.3 | 3.04E−36 |
| Lymphatic endothelial | RP3-523E19.2 | 1.06E−35 |
| Lymphatic endothelial | NTN1 | 2.26E−35 |
| Lymphatic endothelial | PLEKHG1 | 7.67E−35 |
| Lymphatic endothelial | RP11-527H14.2 | 7.79E−35 |
| Lymphatic endothelial | GNAT3 | 8.28E−35 |
| Lymphatic endothelial | CALCRL | 5.97E−34 |
| Lymphatic endothelial | ECSCR | 8.38E−34 |
| Lymphatic endothelial | ASAP1 | 2.23E−33 |
| Lymphatic endothelial | ARHGAP26-AS1 | 2.20E−32 |
| Lymphatic endothelial | RASGRP3 | 6.64E−32 |
| Lymphatic endothelial | EGFL7 | 3.80E−31 |
| Lymphatic endothelial | ZFPM2 | 7.83E−31 |
| Lymphatic endothelial | EPB41L2 | 2.24E−30 |
| Lymphatic endothelial | IL7 | 3.22E−30 |
| Lymphatic endothelial | FLT4 | 6.17E−30 |
| Lymphatic endothelial | PLXDC2 | 6.94E−30 |
| Lymphatic endothelial | SMAD1 | 1.10E−29 |
| Lymphatic endothelial | ADD3 | 1.92E−29 |
| Lymphatic endothelial | DOCK9 | 5.76E−29 |
| Lymphatic endothelial | PRKCH | 1.03E−28 |
| Lymphatic endothelial | RPGR | 5.02E−28 |
| Lymphatic endothelial | CATSPERB | 6.57E−28 |
| Lymphatic endothelial | EFCAB4A | 1.13E−27 |
| Lymphatic endothelial | ZNF521 | 1.27E−27 |
| Lymphatic endothelial | AC139100.2 | 2.43E−27 |
| Lymphatic endothelial | DNAJC18 | 2.77E−27 |
| Lymphatic endothelial | TANC2 | 3.18E−27 |
| Lymphatic endothelial | APP | 3.91E−27 |
| Lymphatic endothelial | RP11-14N7.2 | 6.95E−27 |
| Lymphatic endothelial | KLHL4 | 8.25E−27 |
| Lymphatic endothelial | TSPAN5 | 9.04E−27 |
| Lymphatic endothelial | PLD1 | 2.97E−26 |
| Lymphatic endothelial | DPYSL3 | 7.58E−26 |
| Lymphatic endothelial | STK32B | 7.98E−26 |
| Lymphatic endothelial | CYP8B1 | 1.32E−25 |
| Lymphatic endothelial | RGS16 | 1.86E−25 |
| Lymphatic endothelial | RP11-782C8.5 | 3.62E−24 |
| Lymphatic endothelial | PROX1-AS1 | 4.95E−24 |
| Lymphatic endothelial | SH3BGRL2 | 1.67E−21 |
| Lymphatic endothelial | PANK2 | 3.62E−20 |
| Lymphatic endothelial | SLC9C1 | 3.90E−20 |
| Lymphatic endothelial | STC1 | 4.39E−20 |
| Lymphatic endothelial | DPEP2 | 5.36E−20 |
| Lymphatic endothelial | ARL4A | 3.29E−19 |
| Lymphatic endothelial | SNCG | 6.64E−19 |
| Lymphatic endothelial | LINC01117 | 9.46E−19 |
| Lymphatic endothelial | RP11-435B5.5 | 1.96E−18 |
| Lymphatic endothelial | KBTBD11 | 6.84E−18 |
| Lymphatic endothelial | RP11-327I22.6 | 4.13E−17 |
| Lymphatic endothelial | IGF1 | 1.31E−16 |
| Lymphatic endothelial | ZNF554 | 1.37E−16 |
| Lymphatic endothelial | PTPN3 | 1.39E−16 |
| Lymphatic endothelial | CTD-253611.1 | 3.17E−16 |
| Lymphatic endothelial | KDR | 6.20E−16 |
| Lymphatic endothelial | ART4 | 1.66E−15 |
| Lymphatic endothelial | MAP4K2 | 2.62E−15 |
| Lymphatic endothelial | TSTA3 | 6.35E−15 |
| Lymphatic endothelial | PEAR1 | 4.30E−14 |
| Lymphatic endothelial | RP11-776H12.1 | 4.92E−14 |
| Lymphatic endothelial | SCN3B | 7.62E−14 |
| Lymphatic endothelial | EYA1 | 1.57E−13 |
| Lymphatic endothelial | TAL1 | 7.50E−13 |
| Lymphatic endothelial | ROBO4 | 2.02E−12 |
| Lymphatic endothelial | RP11-423O2.5 | 1.11E−11 |
| Lymphatic endothelial | DKK3 | 1.23E−11 |
| Lymphatic endothelial | RP11-1070N10.4 | 1.44E−11 |
| Lymphatic endothelial | CD200 | 1.13E−10 |
| Lymphatic endothelial | CARD10 | 1.48E−10 |
| Lymphatic endothelial | RP11-318M2.2 | 1.59E−10 |
| Lymphatic endothelial | RHAG | 3.41E−10 |
| Lymphatic endothelial | RNF152 | 8.66E−10 |
| Lymphatic endothelial | GJA1 | 8.85E−10 |
| Lymphatic endothelial | C6orf123 | 1.59E−08 |
| Lymphatic endothelial | C5orf64 | 2.85E−08 |
| Lymphatic endothelial | CTA-221G9.11 | 3.54E−08 |
| Lymphatic endothelial | RP4-640H8.2 | 4.15E−08 |
| Lymphatic endothelial | RP11-728F11.4 | 5.79E−08 |
| Lymphatic endothelial | AC010091.1 | 2.98E−07 |
| Lymphatic endothelial | GRPEL2-AS1 | 7.68E−07 |
| Lymphatic endothelial | LAYN | 1.39E−06 |
| Lymphatic endothelial | TNFAIP8L3 | 1.67E−06 |
| Lymphatic endothelial | EFNA1 | 7.05E−06 |
| Macrophages | RBPJ | 4.86E−178 |
| Macrophages | SRGN | 2.09E−158 |
| Macrophages | MS4A6A | 2.76E−134 |
| Macrophages | F13A1 | 1.07E−132 |
| Macrophages | SAT1 | 8.03E−123 |
| Macrophages | CD163 | 6.13E−114 |
| Macrophages | RBM47 | 6.24E−104 |
| Macrophages | LYVE1 | 1.26E−102 |
| Macrophages | FRMD4B | 1.42E−101 |
| Macrophages | STK17B | 2.09E−95 |
| Macrophages | SRGAP2 | 2.77E−95 |
| Macrophages | ZEB2 | 3.57E−94 |
| Macrophages | RP11-347P5.1 | 1.38E−89 |
| Macrophages | MAN1A1 | 4.97E−88 |
| Macrophages | MSR1 | 3.48E−86 |
| Macrophages | TBXAS1 | 7.73E−84 |
| Macrophages | SLC9A9 | 4.03E−83 |
| Macrophages | FGD2 | 6.09E−80 |
| Macrophages | MAFB | 1.18E−77 |
| Macrophages | SYK | 2.86E−74 |
| Macrophages | CD74 | 1.43E−69 |
| Macrophages | VSIG4 | 8.53E−69 |
| Macrophages | LRRC16A | 8.85E−68 |
| Macrophages | CD14 | 1.76E−65 |
| Macrophages | RP11-343N15.1 | 2.09E−64 |
| Macrophages | MEF2C | 2.31E−63 |
| Macrophages | PTPRC | 3.06E−62 |
| Macrophages | CPM | 4.09E−61 |
| Macrophages | RP11-452H21.1 | 4.76E−57 |
| Macrophages | TYMP | 6.19E−57 |
| Macrophages | CPVL | 6.19E−57 |
| Macrophages | FMN1 | 1.57E−56 |
| Macrophages | STAB1 | 1.63E−56 |
| Macrophages | TG | 2.35E−56 |
| Macrophages | FCGR2B | 2.51E−56 |
| Macrophages | SMAP2 | 1.46E−54 |
| Macrophages | IQGAP2 | 5.41E−54 |
| Macrophages | LILRB5 | 9.77E−54 |
| Macrophages | DPYD | 5.98E−53 |
| Macrophages | MS4A4E | 8.36E−52 |
| Macrophages | ATG7 | 3.20E−51 |
| Macrophages | ANXA1 | 3.43E−51 |
| Macrophages | AMICA1 | 1.13E−50 |
| Macrophages | MS4A4A | 1.42E−50 |
| Macrophages | SLCO2B1 | 4.02E−50 |
| Macrophages | RCSD1 | 2.19E−49 |
| Macrophages | ARHGAP18 | 2.76E−49 |
| Macrophages | ATP8B4 | 6.55E−49 |
| Macrophages | C5AR1 | 1.11E−47 |
| Macrophages | PDGFC | 4.99E−47 |
| Macrophages | CD86 | 7.36E−47 |
| Macrophages | CELF2 | 9.09E−47 |
| Macrophages | RP11-701P16.2 | 1.26E−46 |
| Macrophages | PIK3R5 | 1.90E−46 |
| Macrophages | NAMPT | 3.78E−46 |
| Macrophages | TFRC | 4.28E−43 |
| Macrophages | CLEC7A | 6.58E−43 |
| Macrophages | CTSB | 9.96E−43 |
| Macrophages | HCLS1 | 1.12E−42 |
| Macrophages | SIGLEC1 | 6.21E−42 |
| Macrophages | SLC11A1 | 1.15E−41 |
| Macrophages | PLXDC2 | 3.97E−41 |
| Macrophages | P2RY14 | 1.26E−40 |
| Macrophages | RNF149 | 1.61E−40 |
| Macrophages | DUSP1 | 1.63E−40 |
| Macrophages | TSC22D3 | 3.02E−40 |
| Macrophages | LGMN | 8.29E−40 |
| Macrophages | RGL1 | 1.84E−39 |
| Macrophages | MCL1 | 2.99E−39 |
| Macrophages | CLEC10A | 3.97E−39 |
| Macrophages | SH3TC1 | 4.98E−39 |
| Macrophages | HDAC9 | 1.79E−38 |
| Macrophages | RP11-815J21.4 | 2.63E−38 |
| Macrophages | FTH1 | 5.46E−38 |
| Macrophages | SLC1A3 | 6.44E−38 |
| Macrophages | FCGR2A | 1.25E−37 |
| Macrophages | AKAP13 | 2.98E−37 |
| Macrophages | DOCK8 | 4.33E−37 |
| Macrophages | ARRB2 | 1.50E−36 |
| Macrophages | FPR1 | 2.10E−36 |
| Macrophages | PELI1 | 2.98E−36 |
| Macrophages | CHN2 | 4.02E−36 |
| Macrophages | TGFBI | 7.65E−36 |
| Macrophages | NAIP | 8.18E−36 |
| Macrophages | ACSL1 | 1.09E−35 |
| Macrophages | IRAK3 | 1.83E−35 |
| Macrophages | NCF4 | 3.73E−35 |
| Macrophages | FAM49B | 9.29E−35 |
| Macrophages | C10orf11 | 3.06E−34 |
| Macrophages | MTSS1 | 3.10E−34 |
| Macrophages | RNF144B | 3.32E−34 |
| Macrophages | CSF3R | 3.62E−34 |
| Macrophages | PLTP | 8.93E−34 |
| Macrophages | MKRN3 | 1.34E−33 |
| Macrophages | MYO1F | 3.29E−33 |
| Macrophages | MGAT1 | 3.35E−33 |
| Macrophages | FLI1 | 3.53E−33 |
| Macrophages | SIPA1L1 | 4.96E−33 |
| Macrophages | IL10RA | 5.62E−33 |
| Macrophages | AOAH | 6.68E−33 |
| Macrophages | MRC1L1 | 7.87E−33 |
| Macrophages | CD163L1 | 1.12E−32 |
| Macrophages | LST1 | 7.47E−31 |
| Macrophages | C1orf162 | 7.36E−29 |
| Macrophages | SAMSN1 | 1.43E−28 |
| Macrophages | FCN1 | 2.17E−28 |
| Macrophages | C1QB | 6.05E−28 |
| Macrophages | RP11-553K8.5 | 1.32E−27 |
| Macrophages | THEMIS2 | 1.59E−27 |
| Macrophages | ITGAM | 1.88E−25 |
| Macrophages | EMB | 2.45E−25 |
| Macrophages | MUC6 | 1.59E−24 |
| Macrophages | C1QA | 4.58E−24 |
| Macrophages | MRC1 | 4.97E−24 |
| Macrophages | TNFAIP2 | 5.45E−24 |
| Macrophages | MRVI1-AS1 | 6.81E−24 |
| Macrophages | FAM177B | 5.39E−23 |
| Macrophages | HLA-DQB1 | 4.60E−22 |
| Macrophages | HLA-DQA1 | 7.76E−22 |
| Macrophages | TYROBP | 2.61E−21 |
| Macrophages | SIRPB2 | 4.08E−21 |
| Macrophages | FAM196B | 5.15E−21 |
| Macrophages | CYTH4 | 1.08E−20 |
| Macrophages | MIR142 | 6.89E−20 |
| Macrophages | FOLR2 | 7.09E−20 |
| Macrophages | RNASE1 | 4.40E−19 |
| Macrophages | FMNL1 | 3.39E−18 |
| Macrophages | CD83 | 3.82E−18 |
| Macrophages | LAPTM5 | 5.05E−18 |
| Macrophages | CMKLR1 | 1.49E−17 |
| Macrophages | CTD-2337J16.1 | 1.62E−16 |
| Macrophages | FCER1G | 1.76E−16 |
| Macrophages | MNDA | 5.26E−16 |
| Macrophages | LILRB2 | 6.17E−16 |
| Macrophages | CCL3 | 1.81E−15 |
| Macrophages | CLEC4E | 2.91E−15 |
| Macrophages | LILRB4 | 1.09E−14 |
| Macrophages | PARVG | 1.38E−14 |
| Macrophages | C1QC | 5.06E−14 |
| Macrophages | GPR183 | 1.79E−13 |
| Macrophages | DOK2 | 2.17E−13 |
| Macrophages | EREG | 4.07E−13 |
| Macrophages | MCOLN1 | 1.79E−12 |
| Macrophages | IRF8 | 1.93E−12 |
| Macrophages | SCN1B | 2.85E−12 |
| Macrophages | OVOL3 | 4.87E−12 |
| Macrophages | WAS | 1.12E−10 |
| Macrophages | TLR2 | 1.50E−10 |
| Macrophages | TLR1 | 1.86E−08 |
| Macrophages | HRH2 | 2.55E−08 |
| Macrophages | LILRB3 | 3.76E−08 |
| Macrophages | CD68 | 7.67E−08 |
| Macrophages | UNC93B1 | 9.71E−08 |
| Macrophages | OSCAR | 4.78E−06 |
| Macrophages | CXCL16 | 6.43E−06 |
| Monocytes | FTL | 8.47E−195 |
| Monocytes | TMSB4X | 5.91E−185 |
| Monocytes | FTH1 | 6.81E−176 |
| Monocytes | B2M | 5.64E−165 |
| Monocytes | HLA-DRA | 1.73E−150 |
| Monocytes | CD74 | 2.60E−147 |
| Monocytes | TYROBP | 2.55E−138 |
| Monocytes | S100A9 | 2.66E−135 |
| Monocytes | TPT1 | 1.72E−132 |
| Monocytes | S100A4 | 1.15E−131 |
| Monocytes | RPLP1 | 1.15E−131 |
| Monocytes | OAZ1 | 1.37E−131 |
| Monocytes | GPX1 | 1.71E−129 |
| Monocytes | CST3 | 3.75E−129 |
| Monocytes | FCER1G | 2.65E−128 |
| Monocytes | LGALS1 | 6.87E−126 |
| Monocytes | TMSB10 | 9.09E−126 |
| Monocytes | RPL19 | 4.30E−124 |
| Monocytes | SH3BGRL3 | 6.57E−123 |
| Monocytes | SERF2 | 6.33E−122 |
| Monocytes | CYBA | 4.28E−121 |
| Monocytes | RPL15 | 3.84E−118 |
| Monocytes | S100A11 | 4.24E−118 |
| Monocytes | RPS19 | 7.41E−118 |
| Monocytes | RPS9 | 2.21E−116 |
| Monocytes | RPL13A | 2.21E−116 |
| Monocytes | RPS14 | 3.55E−115 |
| Monocytes | RPL13 | 2.77E−114 |
| Monocytes | MT-CO1 | 2.77E−114 |
| Monocytes | RPL11 | 4.91E−111 |
| Monocytes | RPS27A | 9.78E−111 |
| Monocytes | MT-CO3 | 9.78E−111 |
| Monocytes | NPC2 | 3.53E−110 |
| Monocytes | RPL28 | 4.36E−108 |
| Monocytes | MT-ND1 | 2.48E−107 |
| Monocytes | CFL1 | 8.36E−107 |
| Monocytes | LYZ | 3.59E−104 |
| Monocytes | HLA-DRB1 | 1.65E−103 |
| Monocytes | RPS15 | 7.32E−103 |
| Monocytes | RPS23 | 7.85E−103 |
| Monocytes | RPS12 | 8.44E−103 |
| Monocytes | RPL7 | 8.72E−102 |
| Monocytes | UBA52 | 4.26E−98 |
| Monocytes | PFN1 | 6.58E−95 |
| Monocytes | HLA-DPB1 | 7.54E−95 |
| Monocytes | RPS6 | 8.12E−95 |
| Monocytes | MT-ND4 | 5.15E−94 |
| Monocytes | RPL29 | 1.13E−93 |
| Monocytes | ARPC1B | 1.42E−93 |
| Monocytes | RPS4X | 1.78E−93 |
| Monocytes | S100A6 | 2.24E−93 |
| Monocytes | RPL30 | 3.34E−93 |
| Monocytes | EEF1A1 | 4.04E−93 |
| Monocytes | RPS18 | 6.45E−93 |
| Monocytes | MT-ND2 | 8.31E−93 |
| Monocytes | COX4I1 | 2.07E−92 |
| Monocytes | S100A8 | 4.33E−92 |
| Monocytes | DBI | 1.68E−91 |
| Monocytes | RPL27A | 1.14E−90 |
| Monocytes | RPL10 | 3.80E−90 |
| Monocytes | RPL8 | 6.24E−90 |
| Monocytes | RPLP0 | 2.35E−89 |
| Monocytes | RPS13 | 3.95E−89 |
| Monocytes | RPS7 | 1.01E−87 |
| Monocytes | RPLP2 | 2.99E−87 |
| Monocytes | MT-CYB | 5.20E−87 |
| Monocytes | RPS24 | 7.69E−87 |
| Monocytes | RPS20 | 4.76E−86 |
| Monocytes | RPL6 | 1.60E−85 |
| Monocytes | AIF1 | 5.04E−85 |
| Monocytes | CSTB | 1.69E−84 |
| Monocytes | RPS8 | 5.89E−84 |
| Monocytes | HLA-DPA1 | 3.21E−83 |
| Monocytes | CD63 | 3.52E−83 |
| Monocytes | RPL14 | 7.64E−83 |
| Monocytes | SAT1 | 8.90E−83 |
| Monocytes | EIF1 | 9.53E−83 |
| Monocytes | SRP14 | 1.05E−81 |
| Monocytes | ACTB | 2.70E−81 |
| Monocytes | YBX1 | 4.26E−81 |
| Monocytes | RPL3 | 5.98E−81 |
| Monocytes | MT-CO2 | 8.87E−81 |
| Monocytes | RPS3A | 6.13E−80 |
| Monocytes | RPS5 | 2.27E−79 |
| Monocytes | RPL27 | 4.98E−79 |
| Monocytes | GAPDH | 3.93E−78 |
| Monocytes | FAU | 1.99E−77 |
| Monocytes | PSAP | 4.47E−77 |
| Monocytes | MT-ATP6 | 2.16E−76 |
| Monocytes | TUBA1B | 4.48E−76 |
| Monocytes | RPL34 | 4.48E−76 |
| Monocytes | RPL18 | 8.94E−76 |
| Monocytes | EEF1B2 | 1.44E−75 |
| Monocytes | RPS11 | 1.80E−75 |
| Monocytes | PTPRC | 2.55E−75 |
| Monocytes | RPS2 | 5.03E−75 |
| Monocytes | TSPO | 8.26E−75 |
| Monocytes | RPS3 | 9.37E−75 |
| Monocytes | RPL23A | 1.60E−74 |
| Monocytes | RPL35A | 6.38E−74 |
| Monocytes | LAPTM5 | 4.48E−72 |
| Monocytes | CD48 | 1.73E−64 |
| Monocytes | ATP6V1F | 1.38E−63 |
| Monocytes | PYCARD | 2.15E−62 |
| Monocytes | LGALS3 | 5.63E−61 |
| Monocytes | FXYD5 | 2.59E−59 |
| Monocytes | LY86 | 1.46E−58 |
| Monocytes | LST1 | 2.10E−54 |
| Monocytes | HLA-DRB5 | 4.89E−54 |
| Monocytes | HLA-DQB1 | 2.49E−51 |
| Monocytes | CTSZ | 5.91E−51 |
| Monocytes | C1QC | 7.53E−50 |
| Monocytes | GADD45GIP1 | 2.22E−49 |
| Monocytes | HLA-DQA1 | 1.02E−48 |
| Monocytes | NKG7 | 1.56E−48 |
| Monocytes | RHOG | 2.13E−47 |
| Monocytes | C1QB | 2.45E−46 |
| Monocytes | GPSM3 | 5.02E−44 |
| Monocytes | CD68 | 1.40E−42 |
| Monocytes | HLA-DMB | 2.91E−42 |
| Monocytes | HCST | 7.34E−42 |
| Monocytes | RP11-1143G9.4 | 2.53E−41 |
| Monocytes | C1QA | 4.89E−38 |
| Monocytes | GPNMB | 5.68E−38 |
| Monocytes | FABP5 | 3.04E−36 |
| Monocytes | RNASE6 | 1.78E−35 |
| Monocytes | ZDHHC12 | 3.52E−35 |
| Monocytes | USF2 | 1.27E−33 |
| Monocytes | ARF6 | 3.73E−32 |
| Monocytes | CXCR4 | 3.95E−32 |
| Monocytes | S100A12 | 1.67E−31 |
| Monocytes | EVI2B | 1.75E−27 |
| Monocytes | RGS19 | 4.33E−27 |
| Monocytes | SUPT4H1 | 9.27E−27 |
| Monocytes | UCP2 | 3.25E−24 |
| Monocytes | CLEC10A | 5.74E−23 |
| Monocytes | RNASE2 | 8.79E−22 |
| Monocytes | ORAI3 | 4.86E−21 |
| Monocytes | SEPHS2 | 5.54E−20 |
| Monocytes | SERPINA1 | 9.75E−20 |
| Monocytes | FAM26F | 3.43E−19 |
| Monocytes | METTL7B | 2.65E−18 |
| Monocytes | ARL4C | 2.68E−18 |
| Monocytes | ACP5 | 6.89E−18 |
| Monocytes | IGSF6 | 1.78E−16 |
| Monocytes | MYO1G | 5.44E−16 |
| Monocytes | HMOX1 | 7.08E−16 |
| Monocytes | RP11-290F20.3 | 1.16E−15 |
| Monocytes | FCGR1A | 3.56E−14 |
| Monocytes | CCR1 | 7.76E−14 |
| Monocytes | CST7 | 1.25E−13 |
| Monocytes | CCR2 | 7.72E−13 |
| Monocytes | GAPT | 3.33E−12 |
| Monocytes | CD52 | 3.95E−12 |
| Monocytes | CENPW | 5.13E−12 |
| Monocytes | GCHFR | 9.71E−12 |
| Monocytes | HLA-DQA2 | 1.22E−11 |
| Monocytes | LILRB4 | 1.33E−11 |
| Monocytes | DCK | 4.41E−11 |
| Monocytes | S100B | 1.75E−10 |
| Monocytes | ZNF524 | 2.49E−10 |
| Monocytes | FOLR2 | 5.68E−10 |
| Monocytes | CFP | 1.43E−09 |
| Monocytes | SNX20 | 2.06E−09 |
| Monocytes | MKI67 | 2.22E−09 |
| Monocytes | LACC1 | 2.60E−09 |
| Monocytes | IL1B | 3.19E−09 |
| Monocytes | H2AFX | 1.17E−08 |
| Monocytes | IL8 | 1.23E−08 |
| Monocytes | NFE2 | 2.93E−08 |
| Monocytes | SPP1 | 7.62E−08 |
| Monocytes | HAMP | 1.00E−07 |
| Monocytes | PMAIP1 | 7.39E−07 |
| Smooth muscle | MYH11 | 3.68E−240 |
| Smooth muscle | ACTG2 | 5.72E−193 |
| Smooth muscle | SVIL | 3.29E−178 |
| Smooth muscle | SORBS1 | 2.86E−159 |
| Smooth muscle | CACNA1C | 2.37E−127 |
| Smooth muscle | PRUNE2 | 1.35E−118 |
| Smooth muscle | LPP | 1.05E−112 |
| Smooth muscle | DMD | 7.65E−111 |
| Smooth muscle | MIR145 | 2.27E−110 |
| Smooth muscle | NDE1 | 2.80E−110 |
| Smooth muscle | NT5DC3 | 1.72E−94 |
| Smooth muscle | SYNPO2 | 4.78E−89 |
| Smooth muscle | KCNMA1 | 4.05E−86 |
| Smooth muscle | COL6A2 | 1.17E−85 |
| Smooth muscle | CCBE1 | 1.12E−82 |
| Smooth muscle | PDE4D | 3.50E−82 |
| Smooth muscle | MYL9 | 4.00E−81 |
| Smooth muscle | FBXO32 | 5.23E−77 |
| Smooth muscle | MIR143HG | 2.37E−75 |
| Smooth muscle | FOXP2 | 8.35E−73 |
| Smooth muscle | TPM2 | 1.38E−72 |
| Smooth muscle | RBPMS | 3.04E−72 |
| Smooth muscle | PDZRN4 | 2.60E−70 |
| Smooth muscle | SMTN | 7.64E−70 |
| Smooth muscle | CNN1 | 2.21E−67 |
| Smooth muscle | FLNA | 3.86E−67 |
| Smooth muscle | TPM1 | 8.90E−67 |
| Smooth muscle | CTD-3105H18.18 | 3.92E−64 |
| Smooth muscle | LMOD1 | 3.12E−61 |
| Smooth muscle | LINC00578 | 1.77E−60 |
| Smooth muscle | CALD1 | 1.49E−57 |
| Smooth muscle | ACTA2 | 2.16E−57 |
| Smooth muscle | PDZRN3 | 1.75E−56 |
| Smooth muscle | SLMAP | 3.85E−55 |
| Smooth muscle | MALAT1 | 2.43E−54 |
| Smooth muscle | AC005358.3 | 2.42E−53 |
| Smooth muscle | CACNB2 | 1.38E−52 |
| Smooth muscle | MYLK | 6.11E−50 |
| Smooth muscle | COL6A1 | 2.47E−49 |
| Smooth muscle | PDLIM7 | 3.50E−49 |
| Smooth muscle | DES | 6.05E−49 |
| Smooth muscle | PRKG1 | 3.40E−47 |
| Smooth muscle | SLC8A1 | 1.07E−46 |
| Smooth muscle | DPP6 | 2.93E−44 |
| Smooth muscle | ROR2 | 3.02E−44 |
| Smooth muscle | HDAC4 | 1.46E−43 |
| Smooth muscle | CASKIN1 | 2.00E−41 |
| Smooth muscle | PCDH7 | 2.51E−41 |
| Smooth muscle | RBFOX3 | 7.42E−40 |
| Smooth muscle | NEXN | 2.49E−39 |
| Smooth muscle | BNC2 | 1.18E−38 |
| Smooth muscle | CHRM2 | 3.34E−38 |
| Smooth muscle | CBR4 | 3.55E−38 |
| Smooth muscle | CHRM3 | 6.01E−38 |
| Smooth muscle | PALLD | 6.43E−38 |
| Smooth muscle | SLC8A1-AS1 | 6.58E−36 |
| Smooth muscle | PDK4 | 6.73E−36 |
| Smooth muscle | STAB2 | 8.50E−36 |
| Smooth muscle | MON1B | 9.64E−36 |
| Smooth muscle | GEM | 2.04E−34 |
| Smooth muscle | STT3A-AS1 | 3.02E−34 |
| Smooth muscle | AP001347.6 | 3.02E−34 |
| Smooth muscle | hsa-mir-490 | 3.97E−33 |
| Smooth muscle | ACTN1 | 8.07E−33 |
| Smooth muscle | RP11-611D20.2 | 2.68E−32 |
| Smooth muscle | FHL1 | 3.58E−32 |
| Smooth muscle | CACNA2D1 | 6.79E−32 |
| Smooth muscle | AF001548.5 | 3.60E−31 |
| Smooth muscle | RP11-123O10.4 | 4.40E−31 |
| Smooth muscle | ITGA5 | 8.79E−30 |
| Smooth muscle | MEIS1 | 2.02E−29 |
| Smooth muscle | PARVA | 2.94E−29 |
| Smooth muscle | SPOP | 5.31E−29 |
| Smooth muscle | AC007392.3 | 5.77E−29 |
| Smooth muscle | MSRB3 | 1.08E−28 |
| Smooth muscle | PDLIM3 | 1.27E−28 |
| Smooth muscle | MYOCD | 2.08E−28 |
| Smooth muscle | GPM6A | 6.73E−28 |
| Smooth muscle | FN1 | 9.22E−28 |
| Smooth muscle | TAGLN | 1.30E−27 |
| Smooth muscle | MYL6 | 1.83E−27 |
| Smooth muscle | ATP2B4 | 2.48E−27 |
| Smooth muscle | PPP1R12B | 2.74E−27 |
| Smooth muscle | MEIS2 | 5.82E−27 |
| Smooth muscle | SEMA3A | 2.10E−26 |
| Smooth muscle | COL4A3 | 2.84E−26 |
| Smooth muscle | LHCGR | 8.51E−26 |
| Smooth muscle | ENAH | 1.09E−25 |
| Smooth muscle | SORBS2 | 1.38E−25 |
| Smooth muscle | CSRP1 | 2.85E−25 |
| Smooth muscle | LDB3 | 2.90E−25 |
| Smooth muscle | ITGA1 | 3.08E−25 |
| Smooth muscle | PNCK | 2.56E−24 |
| Smooth muscle | ADAMTS9-AS2 | 2.79E−24 |
| Smooth muscle | AC100830.3 | 7.44E−24 |
| Smooth muscle | CKB | 3.12E−23 |
| Smooth muscle | RP11-370I10.2 | 4.18E−23 |
| Smooth muscle | PARD3B | 1.39E−22 |
| Smooth muscle | AKAP12 | 2.29E−22 |
| Smooth muscle | SLFNL1 | 2.33E−22 |
| Smooth muscle | FGFR2 | 5.10E−22 |
| Smooth muscle | EPHA7 | 5.33E−22 |
| Smooth muscle | SDS | 5.54E−22 |
| Smooth muscle | RP11-619J20.1 | 1.02E−19 |
| Smooth muscle | PSMA8 | 1.30E−19 |
| Smooth muscle | SPEG | 3.31E−19 |
| Smooth muscle | SOGA2 | 9.37E−19 |
| Smooth muscle | PCA3 | 4.62E−18 |
| Smooth muscle | RP11-166P13.4 | 5.22E−18 |
| Smooth muscle | WNK2 | 3.38E−17 |
| Smooth muscle | RP11-374M1.3 | 4.42E−17 |
| Smooth muscle | RP11-413B19.2 | 4.16E−16 |
| Smooth muscle | OCEL1 | 5.59E−16 |
| Smooth muscle | NECAB1 | 2.53E−15 |
| Smooth muscle | SYNM | 2.66E−15 |
| Smooth muscle | ASB2 | 7.83E−15 |
| Smooth muscle | TIGD7 | 5.82E−14 |
| Smooth muscle | FBXL22 | 1.41E−13 |
| Smooth muscle | CTC-529L17.2 | 2.37E−13 |
| Smooth muscle | WFDC1 | 6.33E−13 |
| Smooth muscle | C15orf52 | 1.18E−12 |
| Smooth muscle | HSD17B6 | 2.09E−12 |
| Smooth muscle | EHBP1L1 | 6.24E−12 |
| Smooth muscle | RP11-158I9.5 | 9.79E−12 |
| Smooth muscle | LIPI | 1.38E−11 |
| Smooth muscle | HOXD10 | 6.16E−11 |
| Smooth muscle | RP11-579E24.2 | 2.50E−10 |
| Smooth muscle | RP11-1069G10.1 | 5.29E−10 |
| Smooth muscle | ARRDC4 | 8.03E−10 |
| Smooth muscle | RP11-266N13.2 | 8.39E−10 |
| Smooth muscle | SLC2A4 | 8.48E−10 |
| Smooth muscle | CTD-2313P7.1 | 2.13E−09 |
| Smooth muscle | C20orf166-AS1 | 3.52E−09 |
| Smooth muscle | ADAM11 | 8.20E−09 |
| Smooth muscle | AKAP1 | 1.24E−08 |
| Smooth muscle | NAV2-AS3 | 2.42E−08 |
| Smooth muscle | PSD | 1.07E−07 |
| Smooth muscle | MRVI1 | 2.08E−07 |
| Smooth muscle | MMP3 | 4.94E−07 |
| Smooth muscle | LRTM1 | 5.51E−07 |
| Smooth muscle | CACNA1C-AS1 | 1.91E−06 |
| Smooth muscle | CTC-296K1.4 | 3.12E−06 |
| Smooth muscle | CTC-296K1.3 | 4.58E−06 |
| Smooth muscle | CACNA1H | 6.19E−06 |
| Smooth muscle | FAM83D | 6.34E−06 |
| Smooth muscle | PI15 | 1.19E−05 |
| Smooth muscle | FENDRR | 1.30E−05 |
| Smooth muscle | CTPS1 | 1.30E−05 |
| Smooth muscle | POPDC2 | 1.42E−05 |
| Smooth muscle | AC073635.5 | 2.14E−05 |
| Smooth muscle | RP11-707P20.1 | 3.04E−05 |
| Smooth muscle | RP11-131H24.4 | 7.66E−05 |
| Smooth muscle | ANO5 | 1.38E−04 |
| Smooth muscle | LPA | 3.45E−04 |
| Smooth muscle | TRMT61A | 5.68E−04 |
| Vascular endothelial | LDB2 | 8.82E−186 |
| Vascular endothelial | PTPRB | 1.10E−146 |
| Vascular endothelial | MCTP1 | 1.49E−135 |
| Vascular endothelial | VWF | 6.87E−127 |
| Vascular endothelial | EPAS1 | 9.05E−123 |
| Vascular endothelial | EMP1 | 2.64E−120 |
| Vascular endothelial | RP3-510L9.1 | 2.52E−118 |
| Vascular endothelial | MECOM | 2.63E−112 |
| Vascular endothelial | CTA-276F8.2 | 4.45E−98 |
| Vascular endothelial | PTPRM | 5.55E−93 |
| Vascular endothelial | ARL15 | 2.72E−91 |
| Vascular endothelial | TMTC1 | 1.64E−89 |
| Vascular endothelial | EGFL7 | 5.14E−89 |
| Vascular endothelial | PIK3R3 | 8.57E−89 |
| Vascular endothelial | CYYR1 | 2.62E−87 |
| Vascular endothelial | ANO2 | 1.64E−81 |
| Vascular endothelial | MKL2 | 3.34E−79 |
| Vascular endothelial | LIFR | 1.85E−72 |
| Vascular endothelial | EMCN | 3.40E−72 |
| Vascular endothelial | ID1 | 9.99E−71 |
| Vascular endothelial | PALMD | 1.42E−69 |
| Vascular endothelial | ELMO1-AS1 | 1.18E−67 |
| Vascular endothelial | ERG | 1.59E−67 |
| Vascular endothelial | SPRY1 | 1.02E−65 |
| Vascular endothelial | CXCL2 | 4.01E−64 |
| Vascular endothelial | ELMO1 | 3.94E−63 |
| Vascular endothelial | PITPNC1 | 1.38E−62 |
| Vascular endothelial | ARHGAP31 | 8.80E−61 |
| Vascular endothelial | SPC25 | 3.33E−60 |
| Vascular endothelial | PREX2 | 3.80E−60 |
| Vascular endothelial | ABLIM1 | 2.58E−59 |
| Vascular endothelial | A2M | 5.68E−59 |
| Vascular endothelial | PLCB4 | 1.56E−57 |
| Vascular endothelial | BMPR2 | 1.59E−57 |
| Vascular endothelial | HIPK3 | 4.17E−57 |
| Vascular endothelial | ELTD1 | 1.06E−56 |
| Vascular endothelial | EVA1C | 7.35E−56 |
| Vascular endothelial | NEDD9 | 2.49E−54 |
| Vascular endothelial | AP001597.1 | 1.81E−53 |
| Vascular endothelial | SOCS3 | 2.27E−52 |
| Vascular endothelial | PRKCH | 4.10E−52 |
| Vascular endothelial | TACC1 | 6.58E−52 |
| Vascular endothelial | RIN2 | 2.85E−51 |
| Vascular endothelial | ST6GALNAC3 | 1.36E−48 |
| Vascular endothelial | SLCO2A1 | 1.87E−48 |
| Vascular endothelial | TCF4 | 2.65E−48 |
| Vascular endothelial | TMSB10 | 1.72E−47 |
| Vascular endothelial | ENTPD1-AS1 | 2.09E−47 |
| Vascular endothelial | CTD-3222D19.2 | 3.25E−47 |
| Vascular endothelial | SPARCL1 | 2.31E−46 |
| Vascular endothelial | ZNF385D | 3.21E−46 |
| Vascular endothelial | TPO | 5.21E−45 |
| Vascular endothelial | PLEKHG1 | 4.59E−44 |
| Vascular endothelial | DOCK4 | 5.52E−44 |
| Vascular endothelial | MAGI1 | 7.85E−43 |
| Vascular endothelial | RP1-90G24.10 | 8.01E−42 |
| Vascular endothelial | MYRIP | 3.15E−41 |
| Vascular endothelial | ATP8B1 | 3.84E−40 |
| Vascular endothelial | FLT1 | 5.49E−40 |
| Vascular endothelial | ID3 | 1.48E−39 |
| Vascular endothelial | SASH1 | 5.07E−39 |
| Vascular endothelial | RUNDC3B | 5.62E−39 |
| Vascular endothelial | NPDC1 | 1.78E−38 |
| Vascular endothelial | SYNE2 | 7.09E−38 |
| Vascular endothelial | PKP4 | 1.16E−37 |
| Vascular endothelial | NUAK1 | 1.38E−37 |
| Vascular endothelial | TIMP3 | 1.39E−37 |
| Vascular endothelial | DARC | 1.48E−37 |
| Vascular endothelial | FAM155A | 2.57E−37 |
| Vascular endothelial | GFOD1 | 2.96E−37 |
| Vascular endothelial | SEC14L1 | 1.80E−36 |
| Vascular endothelial | KIAA0355 | 2.91E−36 |
| Vascular endothelial | RP4-678D15.1 | 3.35E−36 |
| Vascular endothelial | RALGAPA2 | 3.36E−36 |
| Vascular endothelial | MSN | 6.72E−36 |
| Vascular endothelial | WNK1 | 7.78E−36 |
| Vascular endothelial | ADAMTS1 | 2.86E−35 |
| Vascular endothelial | ARHGAP26 | 5.46E−35 |
| Vascular endothelial | TM4SF1 | 6.07E−35 |
| Vascular endothelial | EPHA4 | 1.17E−34 |
| Vascular endothelial | RP11-588H23.3 | 1.70E−34 |
| Vascular endothelial | SRGN | 2.05E−34 |
| Vascular endothelial | AC007319.1 | 5.34E−34 |
| Vascular endothelial | RASAL2 | 6.46E−34 |
| Vascular endothelial | MYO1E | 9.22E−34 |
| Vascular endothelial | TSHZ2 | 2.47E−33 |
| Vascular endothelial | AL035610.2 | 8.55E−33 |
| Vascular endothelial | PPP1R16B | 9.39E−33 |
| Vascular endothelial | ZFP36 | 2.66E−32 |
| Vascular endothelial | MEF2C | 2.90E−32 |
| Vascular endothelial | FLI1 | 3.82E−32 |
| Vascular endothelial | TNFRSF10D | 3.85E−32 |
| Vascular endothelial | CRIM1 | 1.57E−31 |
| Vascular endothelial | EDN1 | 1.67E−31 |
| Vascular endothelial | HLA-E | 2.56E−31 |
| Vascular endothelial | RASGRF2 | 3.53E−30 |
| Vascular endothelial | NOTCH4 | 4.84E−30 |
| Vascular endothelial | FOS | 7.95E−30 |
| Vascular endothelial | JUNB | 2.20E−29 |
| Vascular endothelial | PTPRG | 2.28E−29 |
| Vascular endothelial | POSTN | 8.11E−29 |
| Vascular endothelial | TIE1 | 1.82E−27 |
| Vascular endothelial | SOX17 | 5.11E−27 |
| Vascular endothelial | IGFBP3 | 7.90E−27 |
| Vascular endothelial | CD93 | 2.22E−26 |
| Vascular endothelial | TINAGL1 | 3.26E−26 |
| Vascular endothelial | CRIP2 | 1.12E−25 |
| Vascular endothelial | RP11-619L19.1 | 1.15E−25 |
| Vascular endothelial | DPYS | 7.09E−25 |
| Vascular endothelial | LMCD1 | 4.99E−24 |
| Vascular endothelial | VCAM1 | 9.92E−24 |
| Vascular endothelial | CLEC14A | 5.73E−23 |
| Vascular endothelial | CXorf36 | 6.11E−23 |
| Vascular endothelial | ECSCR | 1.16E−22 |
| Vascular endothelial | RPGR | 3.18E−22 |
| Vascular endothelial | CDH5 | 1.46E−21 |
| Vascular endothelial | RAMP3 | 4.79E−21 |
| Vascular endothelial | ADAM15 | 8.21E−21 |
| Vascular endothelial | RAMP2 | 3.83E−20 |
| Vascular endothelial | LINC00847 | 2.44E−19 |
| Vascular endothelial | EFNB2 | 9.40E−19 |
| Vascular endothelial | ELOVL7 | 2.06E−18 |
| Vascular endothelial | BTNL9 | 2.17E−18 |
| Vascular endothelial | THBD | 9.55E−18 |
| Vascular endothelial | VEGFC | 1.09E−17 |
| Vascular endothelial | RAPGEF3 | 1.05E−16 |
| Vascular endothelial | TEK | 2.39E−16 |
| Vascular endothelial | HYAL2 | 4.00E−16 |
| Vascular endothelial | SNCG | 4.38E−16 |
| Vascular endothelial | MEOX2 | 6.81E−16 |
| Vascular endothelial | DLL4 | 6.98E−16 |
| Vascular endothelial | IL6 | 7.04E−16 |
| Vascular endothelial | CTA-134P22.2 | 1.11E−15 |
| Vascular endothelial | ZNF366 | 1.73E−15 |
| Vascular endothelial | GJA5 | 5.18E−15 |
| Vascular endothelial | AQP1 | 1.00E−14 |
| Vascular endothelial | SMAD7 | 1.59E−14 |
| Vascular endothelial | AJ006995.3 | 3.76E−14 |
| Vascular endothelial | CTC-484P3.3 | 3.90E−14 |
| Vascular endothelial | RP11-355F16.1 | 8.32E−14 |
| Vascular endothelial | ATOH8 | 1.57E−13 |
| Vascular endothelial | STC1 | 2.98E−13 |
| Vascular endothelial | ARHGEF15 | 9.54E−13 |
| Vascular endothelial | MAPK11 | 1.02E−12 |
| Vascular endothelial | CX3CL1 | 1.22E−12 |
| Vascular endothelial | GIMAP8 | 1.87E−12 |
| Vascular endothelial | SERPINE1 | 6.31E−12 |
| Vascular endothelial | SHANK3 | 1.10E−11 |
| Vascular endothelial | RP1-29C18.10 | 1.29E−11 |
| Vascular endothelial | AJ239322.3 | 2.08E−11 |
| Vascular endothelial | SELP | 3.48E−11 |
| Vascular endothelial | RP11-778O17.4 | 3.48E−11 |
| Vascular endothelial | SLCO4A1 | 3.94E−11 |
| Vascular endothelial | RP11-188C12.3 | 6.38E−11 |
| Vascular endothelial | RP11-1070N10.4 | 7.09E−11 |
| Vascular endothelial | NPR1 | 1.04E−10 |
| Vascular endothelial | RP11-805F19.2 | 2.07E−10 |
| Vascular endothelial | ESAM | 7.77E−10 |
| Vascular endothelial | KCNJ1 | 9.81E−10 |
| Vascular endothelial | RP5-1121H13.3 | 1.99E−09 |
| Vascular endothelial | SYT15 | 1.85E−08 |
| Vascular endothelial | SLC9A3R2 | 4.28E−08 |
| Vascular endothelial | CASKIN2 | 1.39E−07 |
| Vascular endothelial | CTC-459M5.2 | 5.44E−07 |

TABLE 21

| ident | gene | padjH |
|---|---|---|
| PEMN_1 | RP4-678D15.1 | 5.98E−104 |
| PEMN_1 | TSHZ2 | 6.27E−101 |
| PEMN_1 | RP11-385J1.2 | 2.35E−70 |
| PEMN_1 | ALK | 3.07E−66 |
| PEMN_1 | TMEM132C | 4.93E−65 |
| PEMN_1 | GRID2 | 3.38E−48 |
| PEMN_1 | RP3-399L15.3 | 2.84E−46 |
| PEMN_1 | HPSE2 | 8.30E−44 |
| PEMN_1 | CADPS | 1.66E−43 |
| PEMN_1 | DSCAM | 7.53E−41 |
| PEMN_1 | RBFOX1 | 1.40E−38 |
| PEMN_1 | KCNMB2 | 5.28E−38 |
| PEMN_1 | GPC6 | 1.56E−37 |
| PEMN_1 | XYLT1 | 1.63E−37 |
| PEMN_1 | HS3ST5 | 1.31E−35 |
| PEMN_1 | SLC24A2 | 3.07E−35 |
| PEMN_1 | LSAMP-AS1 | 3.19E−34 |
| PEMN_1 | RP11-76I14.1 | 1.07E−32 |
| PEMN_1 | CHRNA7 | 2.91E−32 |
| PEMN_1 | CTC-575N7.1 | 4.21E−32 |
| PEMN_1 | RP11-15M15.2 | 5.43E−32 |
| PEMN_1 | ADAMTS19 | 9.42E−32 |
| PEMN_1 | LSAMP | 1.18E−31 |
| PEMN_1 | RP11-15M15.1 | 9.77E−31 |
| PEMN_1 | RP11-227F19.1 | 1.75E−30 |
| PEMN_1 | CACNA2D1 | 3.81E−29 |
| PEMN_1 | UNC5D | 8.01E−29 |
| PEMN_1 | BNC2 | 2.07E−28 |
| PEMN_1 | KCNIP4 | 1.59E−25 |
| PEMN_1 | CPNE4 | 8.67E−24 |
| PEMN_1 | LRFN5 | 5.59E−21 |
| PEMN_1 | RYR2 | 1.64E−20 |
| PEMN_1 | DMKN | 7.35E−20 |
| PEMN_1 | THSD7B | 1.41E−19 |
| PEMN_1 | CADM1 | 1.99E−19 |
| PEMN_1 | SLC5A7 | 3.53E−19 |
| PEMN_1 | TPD52L1 | 5.44E−19 |
| PEMN_1 | PLCB4 | 4.19E−18 |
| PEMN_1 | FBP1 | 3.83E−17 |
| PEMN_1 | KCNH5 | 7.20E−17 |
| PEMN_1 | EML5 | 9.89E−17 |
| PEMN_1 | AP000462.2 | 3.03E−16 |
| PEMN_1 | NRG3 | 1.54E−15 |
| PEMN_1 | BICD1 | 5.54E−15 |
| PEMN_1 | SORBS2 | 8.69E−15 |
| PEMN_1 | FRMPD4 | 1.65E−14 |
| PEMN_1 | SNAP25-AS1 | 3.17E−14 |
| PEMN_1 | ARHGEF3 | 4.12E−14 |
| PEMN_1 | ADAMTSL1 | 5.36E−14 |
| PEMN_1 | FRMD4B | 6.77E−14 |
| PEMN_1 | KAZN | 2.17E−13 |
| PEMN_1 | PDE4B | 4.16E−13 |
| PEMN_1 | RBMS3 | 6.66E−13 |
| PEMN_1 | CBS | 9.62E−13 |
| PEMN_1 | HTR4 | 1.13E−12 |
| PEMN_1 | RGS6 | 1.80E−12 |
| PEMN_1 | AP000797.3 | 2.15E−12 |
| PEMN_1 | AL035610.2 | 2.81E−12 |
| PEMN_1 | CLDN11 | 3.35E−12 |
| PEMN_1 | CADM2 | 6.70E−12 |
| PEMN_1 | PPP2R2B | 9.13E−12 |
| PEMN_1 | PSD3 | 1.24E−11 |
| PEMN_1 | BACH2 | 2.07E−11 |
| PEMN_1 | PRICKLE2 | 2.19E−11 |
| PEMN_1 | LRFN2 | 4.55E−11 |
| PEMN_1 | MAST4 | 5.50E−11 |
| PEMN_1 | BRINP2 | 8.70E−11 |
| PEMN_1 | AMPH | 4.09E−10 |
| PEMN_1 | MAML3 | 4.66E−10 |
| PEMN_1 | NRP1 | 8.25E−10 |
| PEMN_1 | DAPK2 | 1.07E−09 |
| PEMN_1 | ABTB2 | 1.07E−09 |
| PEMN_1 | NDUFA4L2 | 1.88E−09 |
| PEMN_1 | AJ006995.3 | 1.91E−09 |
| PEMN_1 | ADAMTS9-AS2 | 3.50E−09 |
| PEMN_1 | RP11-390N6.1 | 3.96E−09 |
| PEMN_1 | SYT6 | 5.10E−09 |
| PEMN_1 | AC009120.6 | 6.34E−09 |
| PEMN_1 | RP11-111E14.1 | 6.44E−09 |
| PEMN_1 | CTD-2576D5.4 | 7.33E−09 |
| PEMN_1 | GPR22 | 1.23E−08 |
| PEMN_1 | SLIT3 | 1.26E−08 |
| PEMN_1 | VCAN | 1.31E−08 |
| PEMN_1 | RP11-383H13.1 | 1.37E−08 |
| PEMN_1 | LHFPL3 | 1.47E−08 |
| PEMN_1 | FBXO48 | 2.11E−08 |
| PEMN_1 | RP4-765H13.1 | 2.12E−08 |
| PEMN_1 | HTR1E | 2.31E−08 |
| PEMN_1 | PTPRR | 2.85E−08 |
| PEMN_1 | EPAS1 | 3.05E−08 |
| PEMN_1 | ZDHHC14 | 3.05E−08 |
| PEMN_1 | STXBP5L | 3.05E−08 |
| PEMN_1 | RP1-34H18.1 | 3.22E−08 |
| PEMN_1 | LPPR5 | 6.37E−08 |
| PEMN_1 | PKNOX2 | 6.49E−08 |
| PEMN_1 | RP11-179K3.2 | 7.13E−08 |
| PEMN_1 | FRK | 7.90E−08 |
| PEMN_1 | PDE4D | 1.13E−07 |
| PEMN_1 | SEMA5A | 1.18E−07 |
| PEMN_1 | AC013463.2 | 1.21E−07 |
| PEMN_1 | GPC6-AS1 | 1.59E−07 |
| PEMN_1 | RHBDL3 | 5.83E−07 |
| PEMN_1 | NNAT | 8.08E−07 |
| PEMN_1 | RSPO2 | 1.72E−06 |
| PEMN_1 | TRPA1 | 3.40E−06 |
| PEMN_1 | NXPH2 | 7.26E−06 |
| PEMN_1 | AP000476.1 | 1.29E−05 |
| PEMN_1 | GLRA3 | 2.92E−05 |
| PEMN_1 | PRELP | 7.72E−05 |
| PEMN_1 | CLEC18A | 8.07E−05 |
| PEMN_1 | RP11-402J6.1 | 8.43E−05 |
| PEMN_1 | ANXA10 | 1.04E−04 |
| PEMN_1 | LINC00682 | 1.14E−04 |
| PEMN_1 | GDNF-AS1 | 2.39E−04 |
| PEMN_1 | RP11-556G22.3 | 3.63E−04 |
| PEMN_1 | F3 | 5.97E−04 |
| PEMN_1 | RP11-804L24.2 | 6.06E−04 |
| PEMN_1 | PDGFRB | 1.24E−03 |
| PEMN_1 | FAM124A | 1.88E−03 |
| PEMN_1 | MYRFL | 1.88E−03 |
| PEMN_1 | ABCC11 | 2.91E−03 |
| PEMN_1 | SULT1C4 | 2.91E−03 |
| PEMN_1 | RP11-669N7.2 | 3.46E−03 |
| PEMN_1 | SMIM10 | 4.36E−03 |
| PEMN_1 | FAM180A | 4.73E−03 |
| PEMN_1 | C9orf24 | 4.93E−03 |
| PEMN_1 | RP11-227H15.4 | 9.97E−03 |
| PEMN_1 | RP11-269G24.3 | 1.11E−02 |
| PEMN_1 | PDZD9 | 1.29E−02 |
| PEMN_1 | HSD11B2 | 1.40E−02 |
| PEMN_1 | FGF10-AS1 | 1.63E−02 |
| PEMN_1 | CTC-419K13.1 | 1.73E−02 |
| PEMN_1 | RP5-944M2.3 | 1.91E−02 |
| PEMN_1 | RP11-2C7.1 | 2.00E−02 |
| PEMN_1 | NPTX1 | 2.03E−02 |
| PEMN_1 | AC010336.1 | 2.08E−02 |
| PEMN_1 | CLEC18B | 2.15E−02 |
| PEMN_1 | LINC01049 | 2.53E−02 |
| PEMN_1 | ZMAT5 | 2.66E−02 |
| PEMN_1 | RP1-37N7.1 | 3.22E−02 |
| PEMN_1 | RP11-384F7.2 | 3.24E−02 |
| PEMN_1 | SCGB1D2 | 3.53E−02 |
| PEMN_1 | RP11-259P1.1 | 3.57E−02 |
| PEMN_1 | RP4-734G22.3 | 3.70E−02 |
| PEMN_1 | ATP4A | 3.78E−02 |
| PEMN_1 | KCNK15 | 3.93E−02 |
| PEMN_1 | KCNE3 | 4.26E−02 |
| PEMN_1 | ABHD14A | 4.57E−02 |
| PEMN_2 | CTA-481E9.4 | 3.84E−49 |
| PEMN_2 | KCNIP4 | 8.65E−49 |
| PEMN_2 | PRKG1 | 3.97E−48 |
| PEMN_2 | CSMD3 | 9.91E−47 |
| PEMN_2 | GALNTL6 | 5.83E−45 |
| PEMN_2 | CHRM2 | 1.37E−43 |
| PEMN_2 | CDH13 | 5.98E−38 |
| PEMN_2 | LSAMP | 1.50E−36 |
| PEMN_2 | MACROD2 | 1.56E−35 |
| PEMN_2 | ZNF804A | 1.21E−34 |
| PEMN_2 | GPC6 | 2.14E−34 |
| PEMN_2 | KCNQ3 | 9.57E−34 |
| PEMN_2 | SLC44A5 | 4.76E−32 |
| PEMN_2 | AC067959.1 | 5.13E−30 |
| PEMN_2 | RALYL | 1.38E−29 |
| PEMN_2 | GRID2 | 2.47E−29 |
| PEMN_2 | BRINP3 | 2.21E−28 |
| PEMN_2 | EFNA5 | 2.30E−28 |
| PEMN_2 | SEMA3D | 4.32E−28 |
| PEMN_2 | PTCHD4 | 2.14E−27 |
| PEMN_2 | NBEA | 1.85E−26 |
| PEMN_2 | CALCRL | 8.68E−26 |
| PEMN_2 | SNTG1 | 3.11E−25 |
| PEMN_2 | BNC2 | 5.49E−25 |
| PEMN_2 | KCNQ5 | 6.49E−25 |
| PEMN_2 | SORCS3 | 1.42E−24 |
| PEMN_2 | hsa-mir-490 | 2.34E−24 |
| PEMN_2 | SLC5A7 | 2.27E−23 |
| PEMN_2 | UNC5D | 4.08E−23 |
| PEMN_2 | SGCZ | 4.75E−23 |
| PEMN_2 | ADAMTS19 | 7.14E−23 |
| PEMN_2 | RYR2 | 3.71E−22 |
| PEMN_2 | COLQ | 3.92E−22 |
| PEMN_2 | SYT1 | 1.03E−21 |
| PEMN_2 | TPD52L1 | 3.90E−21 |
| PEMN_2 | KCNH5 | 6.55E−21 |
| PEMN_2 | LHFPL3 | 8.57E−21 |
| PEMN_2 | GRIA4 | 5.38E−20 |
| PEMN_2 | HS3ST5 | 2.81E−19 |
| PEMN_2 | ST6GALNAC5 | 3.57E−19 |
| PEMN_2 | SLCO3A1 | 5.86E−19 |
| PEMN_2 | ADAMTS9-AS2 | 7.53E−19 |
| PEMN_2 | PDE4D | 1.80E−18 |
| PEMN_2 | RP11-76I14.1 | 2.00E−18 |
| PEMN_2 | SLIT3 | 2.81E−18 |
| PEMN_2 | UNC5C | 8.42E−18 |
| PEMN_2 | ZPLD1 | 9.38E−18 |
| PEMN_2 | ADAMTS9 | 1.04E−17 |
| PEMN_2 | LINC00842 | 1.25E−17 |
| PEMN_2 | GUCY1A3 | 1.34E−17 |
| PEMN_2 | AC074363.1 | 1.89E−17 |
| PEMN_2 | AL035610.2 | 3.02E−17 |
| PEMN_2 | LRRTM3 | 3.70E−17 |
| PEMN_2 | RP11-547I7.1 | 4.07E−17 |
| PEMN_2 | DNM3 | 8.01E−17 |
| PEMN_2 | POSTN | 1.84E−16 |
| PEMN_2 | LBH | 1.88E−16 |
| PEMN_2 | LRFN5 | 2.09E−16 |
| PEMN_2 | SYN3 | 5.28E−16 |
| PEMN_2 | GRIA1 | 9.56E−16 |
| PEMN_2 | CA10 | 2.04E−15 |
| PEMN_2 | DIAPH2 | 2.59E−15 |
| PEMN_2 | PEX5L | 2.73E−15 |
| PEMN_2 | LSAMP-AS1 | 8.91E−15 |
| PEMN_2 | FSTL5 | 1.06E−14 |
| PEMN_2 | NREP | 1.36E−14 |
| PEMN_2 | PTPRD | 1.72E−14 |
| PEMN_2 | MEIS1 | 6.51E−14 |
| PEMN_2 | FAM155A | 8.34E−14 |
| PEMN_2 | CADPS | 1.02E−13 |
| PEMN_2 | TIMP3 | 1.15E−13 |
| PEMN_2 | IL15 | 1.15E−13 |
| PEMN_2 | RP11-298D21.1 | 4.69E−13 |
| PEMN_2 | SEMA6D | 6.46E−13 |
| PEMN_2 | DPP6 | 6.93E−13 |
| PEMN_2 | RP11-227F19.1 | 8.14E−13 |
| PEMN_2 | BAI3 | 1.19E−12 |
| PEMN_2 | PIEZO2 | 1.22E−12 |
| PEMN_2 | FBXO48 | 1.68E−12 |
| PEMN_2 | DIAPH2-AS1 | 3.62E−12 |
| PEMN_2 | CCBE1 | 3.80E−12 |
| PEMN_2 | MAGI2 | 4.79E−12 |
| PEMN_2 | TRPA1 | 7.54E−12 |
| PEMN_2 | RP3-399L15.3 | 1.02E−11 |
| PEMN_2 | PRICKLE2 | 1.14E−11 |
| PEMN_2 | HTR4 | 1.17E−11 |
| PEMN_2 | COL5A1 | 1.42E−11 |
| PEMN_2 | UNC79 | 1.42E−11 |
| PEMN_2 | SLC8A1 | 1.54E−11 |
| PEMN_2 | STAC | 2.08E−11 |
| PEMN_2 | RP11-136K7.2 | 3.23E−11 |
| PEMN_2 | PRB2 | 4.60E−11 |
| PEMN_2 | AC007319.1 | 5.16E−11 |
| PEMN_2 | GABRG3 | 1.13E−10 |
| PEMN_2 | KCNS3 | 1.69E−10 |
| PEMN_2 | CHL1 | 2.13E−10 |
| PEMN_2 | CADM1 | 2.30E−10 |
| PEMN_2 | HTR3A | 6.63E−10 |
| PEMN_2 | CTC-575N7.1 | 8.86E−10 |
| PEMN_2 | TOX | 1.38E−09 |
| PEMN_2 | TLL2 | 5.08E−09 |
| PEMN_2 | RGS4 | 8.65E−09 |
| PEMN_2 | CORO6 | 1.65E−08 |
| PEMN_2 | RP11-707P20.1 | 1.78E−08 |
| PEMN_2 | CTD-3253I12.1 | 2.30E−08 |
| PEMN_2 | RP11-556G22.3 | 2.42E−08 |
| PEMN_2 | HTR3B | 5.78E−08 |
| PEMN_2 | AASS | 9.83E−08 |
| PEMN_2 | AJ239322.3 | 2.01E−07 |
| PEMN_2 | GHSR | 2.69E−07 |
| PEMN_2 | DLX6-AS1 | 5.08E−07 |
| PEMN_2 | RP11-429O1.1 | 5.41E−07 |
| PEMN_2 | RP11-162D9.3 | 5.90E−07 |
| PEMN_2 | RP5-952N6.1 | 7.66E−07 |
| PEMN_2 | PHLDA1 | 1.14E−06 |
| PEMN_2 | HTR7 | 1.61E−06 |
| PEMN_2 | SMAD7 | 2.23E−06 |
| PEMN_2 | RP11-362F19.1 | 5.93E−06 |
| PEMN_2 | PNOC | 7.06E−06 |
| PEMN_2 | SLCO1C1 | 4.98E−05 |
| PEMN_2 | MVB12A | 4.98E−05 |
| PEMN_2 | MAB21L2 | 5.08E−05 |
| PEMN_2 | RP11-435O5.2 | 9.28E−05 |
| PEMN_2 | RP11-17L5.4 | 1.02E−04 |
| PEMN_2 | ANXA1 | 1.16E−04 |
| PEMN_2 | TMEM100 | 2.16E−04 |
| PEMN_2 | RP11-543D5.1 | 3.44E−04 |
| PEMN_2 | KIAA1024L | 4.44E−04 |
| PEMN_2 | HES7 | 8.96E−04 |
| PEMN_2 | C12orf39 | 1.12E−03 |
| PEMN_2 | DLX6 | 1.25E−03 |
| PEMN_2 | RP11-168O10.6 | 1.33E−03 |
| PEMN_2 | LINC00494 | 2.31E−03 |
| PEMN_2 | TMEM133 | 2.44E−03 |
| PEMN_2 | B3GALT1 | 4.23E−03 |
| PEMN_2 | NOC3L | 4.36E−03 |
| PEMN_2 | EVPL | 6.05E−03 |
| PEMN_2 | CTC-558O2.1 | 9.80E−03 |
| PEMN_2 | RP11-923I11.4 | 1.48E−02 |
| PEMN_2 | HGF | 1.48E−02 |
| PEMN_2 | RP11-284H19.1 | 1.65E−02 |
| PEMN_2 | HHLA1 | 1.69E−02 |
| PEMN_2 | GPR35 | 2.06E−02 |
| PEMN_2 | ITGB5-AS1 | 2.12E−02 |
| PEMN_2 | CBR1 | 2.31E−02 |
| PEMN_2 | SSMEM1 | 2.45E−02 |
| PEMN_2 | AC023115.2 | 2.50E−02 |
| PEMN_2 | EFNB3 | 3.18E−02 |
| PEMN_2 | C5orf47 | 3.53E−02 |
| PEMN_2 | RP11-107D24.2 | 3.82E−02 |
| PEMN_2 | SLC5A9 | 4.27E−02 |
| PEMN_2 | CTD-3023L14.2 | 4.28E−02 |
| PEMN_2 | CTB-178M22.1 | 4.34E−02 |
| PEMN_2 | AL136376.1 | 4.42E−02 |
| PIMN_1 | NOS1 | 1.66E−25 |
| PIMN_1 | DGKB | 1.11E−23 |
| PIMN_1 | TMTC2 | 2.21E−19 |
| PIMN_1 | EPB41L3 | 5.46E−19 |
| PIMN_1 | LPHN3 | 4.37E−17 |
| PIMN_1 | DCC | 1.83E−16 |
| PIMN_1 | ALCAM | 9.22E−16 |
| PIMN_1 | SNTB1 | 2.98E−15 |
| PIMN_1 | ARHGAP26 | 3.60E−15 |
| PIMN_1 | DCLK1 | 6.34E−15 |
| PIMN_1 | PRKCE | 7.25E−15 |
| PIMN_1 | HDAC9 | 1.27E−14 |
| PIMN_1 | LDLRAD3 | 1.64E−14 |
| PIMN_1 | ST18 | 2.55E−14 |
| PIMN_1 | TCTEX1D1 | 3.98E−14 |
| PIMN_1 | NLGN1 | 4.96E−14 |
| PIMN_1 | PHYHIPL | 2.49E−13 |
| PIMN_1 | CNTNAP5 | 2.49E−13 |
| PIMN_1 | ALDH1A2 | 3.62E−13 |
| PIMN_1 | GAL | 3.47E−12 |
| PIMN_1 | FLRT2 | 4.44E−12 |
| PIMN_1 | GUCY1A2 | 6.31E−12 |
| PIMN_1 | SLIT2 | 7.84E−12 |
| PIMN_1 | RP1-15D23.2 | 7.84E−12 |
| PIMN_1 | TPST1 | 6.93E−11 |
| PIMN_1 | PTPRG | 4.72E−10 |
| PIMN_1 | CTNNA2 | 2.97E−09 |
| PIMN_1 | RP11-286N3.2 | 4.60E−09 |
| PIMN_1 | AC068533.7 | 4.67E−09 |
| PIMN_1 | TRHDE | 8.80E−09 |
| PIMN_1 | MYRIP | 8.80E−09 |
| PIMN_1 | GLDN | 9.45E−09 |
| PIMN_1 | ODAM | 1.03E−08 |
| PIMN_1 | DMD | 1.14E−08 |
| PIMN_1 | PPAPDC1A | 1.48E−08 |
| PIMN_1 | ASL | 3.45E−08 |
| PIMN_1 | OPRD1 | 7.81E−08 |
| PIMN_1 | TMEM108 | 8.22E−08 |
| PIMN_1 | MAN1A1 | 8.22E−08 |
| PIMN_1 | FOXO3 | 8.72E−08 |
| PIMN_1 | TMEM163 | 9.68E−08 |
| PIMN_1 | MSI2 | 9.68E−08 |
| PIMN_1 | FAM78B | 9.96E−08 |
| PIMN_1 | RP11-1084J3.4 | 9.96E−08 |
| PIMN_1 | SCML4 | 1.16E−07 |
| PIMN_1 | KCNJ5 | 1.68E−07 |
| PIMN_1 | TOX | 1.68E−07 |
| PIMN_1 | KCNC2 | 1.87E−07 |
| PIMN_1 | PDE1C | 2.02E−07 |
| PIMN_1 | MAGI1 | 8.38E−07 |
| PIMN_1 | ADD3 | 9.09E−07 |
| PIMN_1 | NPY | 1.07E−06 |
| PIMN_1 | EML6 | 1.07E−06 |
| PIMN_1 | CIT | 1.11E−06 |
| PIMN_1 | GABRB3 | 1.28E−06 |
| PIMN_1 | PLCB4 | 1.35E−06 |
| PIMN_1 | PTPRE | 1.35E−06 |
| PIMN_1 | KCNG3 | 1.53E−06 |
| PIMN_1 | WIPF1 | 1.61E−06 |
| PIMN_1 | PAG1 | 1.69E−06 |
| PIMN_1 | AKAP6 | 1.69E−06 |
| PIMN_1 | FMNL2 | 1.87E−06 |
| PIMN_1 | TCF4 | 2.46E−06 |
| PIMN_1 | CHD7 | 2.53E−06 |
| PIMN_1 | RBFOX2 | 2.55E−06 |
| PIMN_1 | TANC1 | 2.69E−06 |
| PIMN_1 | SAMD4A | 2.96E−06 |
| PIMN_1 | SLC4A4 | 3.07E−06 |
| PIMN_1 | ETV1 | 4.22E−06 |
| PIMN_1 | PDE1A | 5.01E−06 |
| PIMN_1 | KIAA0319 | 5.10E−06 |
| PIMN_1 | PAM | 1.14E−05 |
| PIMN_1 | NEAT1 | 1.14E−05 |
| PIMN_1 | NFIA | 1.15E−05 |
| PIMN_1 | SORCS1 | 1.26E−05 |
| PIMN_1 | ACTN1 | 1.27E−05 |
| PIMN_1 | GFRA1 | 1.64E−05 |
| PIMN_1 | CREB5 | 2.22E−05 |
| PIMN_1 | ANKRD44 | 2.36E−05 |
| PIMN_1 | PPM1H | 2.67E−05 |
| PIMN_1 | DCBLD2 | 2.67E−05 |
| PIMN_1 | PLCB1 | 2.68E−05 |
| PIMN_1 | ANK3 | 2.68E−05 |
| PIMN_1 | KIF1B | 2.89E−05 |
| PIMN_1 | FHIT | 3.31E−05 |
| PIMN_1 | PLS3 | 4.68E−05 |
| PIMN_1 | ARHGEF28 | 4.68E−05 |
| PIMN_1 | PPP2R5C | 5.21E−05 |
| PIMN_1 | FRMD5 | 5.34E−05 |
| PIMN_1 | MAP3K4 | 7.34E−05 |
| PIMN_1 | SRGAP1 | 9.31E−05 |
| PIMN_1 | SMPD3 | 1.04E−04 |
| PIMN_1 | ASS1 | 1.04E−04 |
| PIMN_1 | DST | 1.04E−04 |
| PIMN_1 | RPRML | 1.40E−04 |
| PIMN_1 | CDK6 | 1.60E−04 |
| PIMN_1 | ZEB2 | 1.64E−04 |
| PIMN_1 | TSPAN11 | 1.72E−04 |
| PIMN_1 | ELL2 | 1.95E−04 |
| PIMN_1 | NFIX | 2.35E−04 |
| PIMN_1 | LFNG | 2.45E−04 |
| PIMN_1 | CNN2 | 2.52E−04 |
| PIMN_1 | UPF1 | 4.83E−04 |
| PIMN_1 | PDGFB | 9.71E−04 |
| PIMN_1 | AQP9 | 1.10E−03 |
| PIMN_1 | STRIP2 | 1.11E−03 |
| PIMN_1 | LAMA5 | 1.17E−03 |
| PIMN_1 | PLCH2 | 1.24E−03 |
| PIMN_1 | CAPN15 | 1.55E−03 |
| PIMN_1 | GALNT2 | 1.86E−03 |
| PIMN_1 | ROPN1L | 1.98E−03 |
| PIMN_1 | CDSN | 3.04E−03 |
| PIMN_1 | GAD2 | 3.12E−03 |
| PIMN_1 | NTF3 | 3.82E−03 |
| PIMN_1 | RP11-453M23.1 | 4.59E−03 |
| PIMN_1 | KCNK17 | 4.93E−03 |
| PIMN_1 | PALD1 | 5.67E−03 |
| PIMN_1 | GABRA4 | 6.13E−03 |
| PIMN_1 | MUC6 | 7.30E−03 |
| PIMN_1 | RP11-993B23.3 | 7.40E−03 |
| PIMN_1 | AC007292.4 | 7.40E−03 |
| PIMN_1 | LURAP1L | 7.40E−03 |
| PIMN_1 | SOHLH1 | 8.53E−03 |
| PIMN_1 | SRPK3 | 9.78E−03 |
| PIMN_1 | FOSL2 | 1.07E−02 |
| PIMN_1 | CACNA1I | 1.09E−02 |
| PIMN_1 | EPPK1 | 1.11E−02 |
| PIMN_1 | RP11-318M2.2 | 1.13E−02 |
| PIMN_1 | HTR2A | 1.25E−02 |
| PIMN_1 | RP11-451M19.3 | 1.28E−02 |
| PIMN_1 | RP11-707A18.1 | 1.33E−02 |
| PIMN_1 | GATA4 | 1.45E−02 |
| PIMN_1 | SLC22A1 | 1.48E−02 |
| PIMN_1 | RP11-23P13.6 | 1.53E−02 |
| PIMN_1 | RP11-631F7.1 | 1.56E−02 |
| PIMN_1 | TNFRSF25 | 1.58E−02 |
| PIMN_1 | RP11-320N7.2 | 1.69E−02 |
| PIMN_1 | MFI2 | 1.78E−02 |
| PIMN_1 | IMPA2 | 1.83E−02 |
| PIMN_1 | DYTN | 2.15E−02 |
| PIMN_1 | RP11-431M7.2 | 2.19E−02 |
| PIMN_1 | LINC01091 | 2.39E−02 |
| PIMN_1 | CTC-546K23.1 | 2.45E−02 |
| PIMN_1 | RP11-264B14.1 | 2.96E−02 |
| PIMN_1 | RP11-766N7.3 | 3.03E−02 |
| PIMN_1 | RP11-944C7.1 | 3.31E−02 |
| PIMN_1 | FAM126A | 3.37E−02 |
| PIMN_1 | AC022182.1 | 3.47E−02 |
| PIMN_1 | SPRY2 | 3.48E−02 |
| PIMN_1 | DNAAF3 | 3.56E−02 |
| PIMN_1 | RP11-479J7.2 | 3.57E−02 |
| PIMN_1 | RP11-713M15.1 | 3.65E−02 |
| PIMN_1 | NPTX2 | 3.72E−02 |
| PIMN_1 | RP3-467L1.4 | 3.73E−02 |
| PIMN_1 | RP11-173P15.7 | 3.73E−02 |
| PIMN_1 | MED9 | 3.74E−02 |
| PIMN_1 | RP11-327L3.3 | 3.77E−02 |
| PIMN_1 | CILP | 3.88E−02 |
| PIMN_1 | ZNF610 | 4.05E−02 |
| PIMN_1 | GCGR | 4.24E−02 |
| PIMN_1 | AP000640.10 | 4.44E−02 |
| PIMN_1 | OSTN | 4.73E−02 |
| PIMN_1 | MYCL | 4.91E−02 |
| PIMN_2 | MYH11 | 7.96E−105 |
| PIMN_2 | ACTG2 | 2.73E−70 |
| PIMN_2 | RBPMS | 2.80E−54 |
| PIMN_2 | SORBS1 | 2.80E−54 |
| PIMN_2 | SVIL | 8.75E−53 |
| PIMN_2 | LPP | 8.95E−52 |
| PIMN_2 | NDE1 | 3.58E−50 |
| PIMN_2 | COL6A2 | 5.92E−50 |
| PIMN_2 | MIR145 | 8.68E−45 |
| PIMN_2 | TPM2 | 2.63E−43 |
| PIMN_2 | FOXP2 | 7.53E−42 |
| PIMN_2 | NT5DC3 | 1.37E−40 |
| PIMN_2 | TPM1 | 1.78E−40 |
| PIMN_2 | FBXO32 | 3.13E−37 |
| PIMN_2 | PDK4 | 6.90E−37 |
| PIMN_2 | CTD-3105H18.18 | 3.99E−34 |
| PIMN_2 | LMOD1 | 4.14E−32 |
| PIMN_2 | CALD1 | 4.13E−31 |
| PIMN_2 | MIR143HG | 1.28E−29 |
| PIMN_2 | MYL9 | 9.41E−29 |
| PIMN_2 | RP11-611D20.2 | 4.74E−28 |
| PIMN_2 | PDZRN4 | 1.21E−27 |
| PIMN_2 | CNN1 | 1.40E−27 |
| PIMN_2 | ARHGAP6 | 4.76E−27 |
| PIMN_2 | SMTN | 4.92E−26 |
| PIMN_2 | ROR2 | 4.92E−26 |
| PIMN_2 | FLNA | 5.40E−26 |
| PIMN_2 | ITGA1 | 8.13E−26 |
| PIMN_2 | STAB2 | 1.72E−25 |
| PIMN_2 | ZBTB16 | 3.42E−25 |
| PIMN_2 | ACTA2 | 4.54E−25 |
| PIMN_2 | SPARCL1 | 7.19E−24 |
| PIMN_2 | MEIS2 | 1.02E−23 |
| PIMN_2 | ITGA5 | 6.84E−23 |
| PIMN_2 | HIF3A | 1.25E−22 |
| PIMN_2 | NEXN | 2.90E−22 |
| PIMN_2 | COL6A1 | l.00E−21 |
| PIMN_2 | LINC00578 | 1.47E−21 |
| PIMN_2 | HDAC4 | 1.11E−20 |
| PIMN_2 | FKBP5 | 1.88E−20 |
| PIMN_2 | AC005358.3 | 2.89E−20 |
| PIMN_2 | CBR4 | 6.19E−20 |
| PIMN_2 | MYLK | 1.53E−19 |
| PIMN_2 | DES | 1.97E−19 |
| PIMN_2 | FAM129A | 3.25E−19 |
| PIMN_2 | CCBE1 | 3.25E−19 |
| PIMN_2 | AF001548.5 | 3.74E−19 |
| PIMN_2 | MGST1 | 1.82E−18 |
| PIMN_2 | COL4A2 | 1.03E−17 |
| PIMN_2 | PDLIM7 | 1.03E−17 |
| PIMN_2 | SEMA3A | 1.09E−17 |
| PIMN_2 | PGM5 | 4.06E−17 |
| PIMN_2 | PDZRN3 | 4.42E−17 |
| PIMN_2 | IGFBP7 | 2.33E−16 |
| PIMN_2 | GNG12-AS1 | 3.45E−16 |
| PIMN_2 | BTG2 | 3.83E−16 |
| PIMN_2 | MBNL1 | 3.83E−16 |
| PIMN_2 | PDLIM3 | 5.61E−16 |
| PIMN_2 | TNC | 8.44E−16 |
| PIMN_2 | GPM6A | 2.55E−15 |
| PIMN_2 | FN1 | 3.62E−15 |
| PIMN_2 | SLMAP | 4.18E−15 |
| PIMN_2 | ETV6 | 4.45E−15 |
| PIMN_2 | TXNIP | 4.91E−15 |
| PIMN_2 | PALLD | 5.04E−15 |
| PIMN_2 | COL1A1 | 9.21E−15 |
| PIMN_2 | ZFP36L1 | 1.45E−14 |
| PIMN_2 | AP001347.6 | 1.57E−14 |
| PIMN_2 | FOXP1 | 2.26E−14 |
| PIMN_2 | TAGLN | 2.48E−14 |
| PIMN_2 | ITPKB-AS1 | 3.14E−14 |
| PIMN_2 | PARD3 | 3.14E−14 |
| PIMN_2 | PARD3B | 5.10E−14 |
| PIMN_2 | RBFOX3 | 6.70E−14 |
| PIMN_2 | TPM4 | 8.01E−14 |
| PIMN_2 | SYNPO2 | 1.94E−13 |
| PIMN_2 | FHL1 | 2.84E−13 |
| PIMN_2 | PARVA | 3.06E−13 |
| PIMN_2 | MON1B | 7.60E−13 |
| PIMN_2 | CRISPLD2 | 7.91E−13 |
| PIMN_2 | DUSP1 | 1.88E−12 |
| PIMN_2 | RP11-242P2.1 | 2.38E−12 |
| PIMN_2 | LHCGR | 2.66E−12 |
| PIMN_2 | NID1 | 4.36E−12 |
| PIMN_2 | NRXN3 | 6.41E−12 |
| PIMN_2 | PBX1 | 7.28E−12 |
| PIMN_2 | FBXL7 | 8.03E−12 |
| PIMN_2 | MYOF | 8.55E−12 |
| PIMN_2 | CACNA1C | 8.86E−12 |
| PIMN_2 | FAM196A | 9.11E−12 |
| PIMN_2 | STT3A-AS1 | 9.46E−12 |
| PIMN_2 | PRUNE2 | 1.82E−11 |
| PIMN_2 | ITIH5 | 1.97E−11 |
| PIMN_2 | COL6A3 | 2.06E−11 |
| PIMN_2 | MSRB3 | 2.16E−11 |
| PIMN_2 | MMP3 | 2.20E−11 |
| PIMN_2 | MID1 | 2.24E−11 |
| PIMN_2 | TBC1D1 | 2.39E−11 |
| PIMN_2 | STK38L | 3.16E−11 |
| PIMN_2 | RP11-166P13.4 | 3.25E−11 |
| PIMN_2 | C20orf166-AS1 | 1.76E−10 |
| PIMN_2 | HOXA11-AS | 2.86E−10 |
| PIMN_2 | KCNMB1 | 4.80E−10 |
| PIMN_2 | CTC-529L17.2 | 2.64E−09 |
| PIMN_2 | AC007401.2 | 4.13E−09 |
| PIMN_2 | FBXL22 | 7.87E−09 |
| PIMN_2 | HSD17B6 | 9.16E−09 |
| PIMN_2 | BCL11A | 1.31E−08 |
| PIMN_2 | MT1E | 1.95E−08 |
| PIMN_2 | SRPX2 | 3.02E−08 |
| PIMN_2 | SOCS3 | 5.29E−08 |
| PIMN_2 | RP11-413B19.2 | 9.82E−08 |
| PIMN_2 | EMP1 | 1.95E−07 |
| PIMN_2 | HOXD10 | 2.82E−07 |
| PIMN_2 | CTC-510F12.2 | 3.32E−07 |
| PIMN_2 | GADD45B | 5.26E−07 |
| PIMN_2 | TINAGL1 | 1.16E−06 |
| PIMN_2 | RP11-242P2.2 | 1.43E−06 |
| PIMN_2 | SDC4 | 2.65E−06 |
| PIMN_2 | MRVI1 | 3.94E−06 |
| PIMN_2 | SLC2A4 | 4.29E−06 |
| PIMN_2 | LIPI | 8.43E−06 |
| PIMN_2 | NFKB2 | 2.73E−05 |
| PIMN_2 | RP11-286H15.1 | 7.64E−05 |
| PIMN_2 | AOC3 | 7.83E−05 |
| PIMN_2 | RP11-893F2.13 | 1.45E−04 |
| PIMN_2 | AC010524.4 | 1.52E−04 |
| PIMN_2 | THBS1 | 2.55E−04 |
| PIMN_2 | SERPINA5 | 4.65E−04 |
| PIMN_2 | C8orf4 | 5.25E−04 |
| PIMN_2 | TNFAIP3 | 7.02E−04 |
| PIMN_2 | MS4A6A | 1.03E−03 |
| PIMN_2 | ADH6 | 1.20E−03 |
| PIMN_2 | CTC-296K1.3 | 1.47E−03 |
| PIMN_2 | ZCCHC24 | 1.77E−03 |
| PIMN_2 | RHOU | 4.12E−03 |
| PIMN_2 | RP11-347P5.1 | 4.81E−03 |
| PIMN_2 | PROX1-AS1 | 5.71E−03 |
| PIMN_2 | FKBP10 | 7.28E−03 |
| PIMN_2 | CXCL2 | 7.33E−03 |
| PIMN_2 | ARID5A | 7.60E−03 |
| PIMN_2 | PPIC | 7.99E−03 |
| PIMN_2 | MASP1 | 8.36E−03 |
| PIMN_2 | CD163 | 8.94E−03 |
| PIMN_2 | ACKR3 | 9.13E−03 |
| PIMN_2 | SDPR | 1.05E−02 |
| PIMN_2 | ROBO3 | 1.08E−02 |
| PIMN_2 | RP11-440I14.2 | 1.50E−02 |
| PIMN_2 | NCKAP1L | 1.63E−02 |
| PIMN_2 | TRPC5OS | 1.65E−02 |
| PIMN_2 | RP11-326C3.12 | 1.87E−02 |
| PIMN_2 | NFE4 | 2.12E−02 |
| PIMN_2 | FENDRR | 2.35E−02 |
| PIMN_2 | RP11-343K8.3 | 2.40E−02 |
| PIMN_2 | HOXD9 | 3.21E−02 |
| PIMN_2 | IL6 | 3.41E−02 |
| PIMN_2 | CLCF1 | 4.18E−02 |
| PIMN_2 | CCND1 | 4.24E−02 |
| PIMN_2 | AC093639.1 | 4.75E−02 |
| PIMN_2 | AP001053.11 | 4.81E−02 |
| PIMN_2 | RP11-309L24.2 | 4.81E−02 |
| PIMN_2 | HES1 | 4.90E−02 |
| PIMN_3 | PDE1A | 2.78E−28 |
| PIMN_3 | FSTL5 | 2.87E−27 |
| PIMN_3 | KCNB2 | 4.91E−27 |
| PIMN_3 | RP11-348J24.2 | 8.13E−26 |
| PIMN_3 | ASS1 | 3.56E−25 |
| PIMN_3 | IQCJ-SCHIP1 | 8.16E−25 |
| PIMN_3 | ERBB4 | 1.24E−22 |
| PIMN_3 | RP11-661P17.1 | 2.31E−21 |
| PIMN_3 | CARTPT | 4.00E−19 |
| PIMN_3 | NPNT | 5.99E−19 |
| PIMN_3 | NOS1 | 3.23E−18 |
| PIMN_3 | SLC4A4 | 1.66E−15 |
| PIMN_3 | NTNG1 | 9.18E−15 |
| PIMN_3 | PCDH15 | 5.11E−13 |
| PIMN_3 | KCND2 | 8.52E−13 |
| PIMN_3 | CDH2 | 1.48E−12 |
| PIMN_3 | SYN3 | 1.57E−12 |
| PIMN_3 | KIAA1217 | 4.59E−12 |
| PIMN_3 | CNTNAP5 | 1.14E−11 |
| PIMN_3 | HECW1 | 1.22E−11 |
| PIMN_3 | KCNC1 | 8.29E−11 |
| PIMN_3 | CSGALNACT1 | 8.29E−11 |
| PIMN_3 | PARVB | 9.91E−11 |
| PIMN_3 | NRG3 | 1.13E−10 |
| PIMN_3 | PTPRK | 1.26E−10 |
| PIMN_3 | FGF14 | 2.06E−10 |
| PIMN_3 | NRXN1 | 2.21E−10 |
| PIMN_3 | ALCAM | 2.48E−10 |
| PIMN_3 | KLHL1 | 6.87E−10 |
| PIMN_3 | NCAM2 | 8.21E−10 |
| PIMN_3 | KCNH7 | 9.31E−10 |
| PIMN_3 | AP001604.3 | 1.05E−09 |
| PIMN_3 | CHRM3 | 2.63E−09 |
| PIMN_3 | TIMP3 | 3.48E−09 |
| PIMN_3 | THSD4 | 6.98E−09 |
| PIMN_3 | ANXA1 | 6.99E−09 |
| PIMN_3 | AL035610.2 | 6.99E−09 |
| PIMN_3 | VIP | 9.94E−09 |
| PIMN_3 | PTGIR | 1.24E−08 |
| PIMN_3 | FLRT2 | 2.68E−08 |
| PIMN_3 | CNTNAP3B | 2.68E−08 |
| PIMN_3 | SOBP | 3.81E−08 |
| PIMN_3 | RP11-133F8.2 | 3.93E−08 |
| PIMN_3 | LTK | 5.42E−08 |
| PIMN_3 | AC007740.1 | 8.97E−08 |
| PIMN_3 | KCNT2 | 8.99E−08 |
| PIMN_3 | B4GALT6 | 1.04E−07 |
| PIMN_3 | ENTPD3 | 1.24E−07 |
| PIMN_3 | TNR | 1.42E−07 |
| PIMN_3 | FAM155A | 1.55E−07 |
| PIMN_3 | NECAB1 | 1.58E−07 |
| PIMN_3 | NGB | 1.58E−07 |
| PIMN_3 | ADCYAP1 | 2.34E−07 |
| PIMN_3 | CNGB1 | 3.65E−07 |
| PIMN_3 | RP11-260M19.2 | 4.31E−07 |
| PIMN_3 | KHDRBS2 | 4.94E−07 |
| PIMN_3 | AP001605.4 | 5.72E−07 |
| PIMN_3 | MARCH1 | 7.17E−07 |
| PIMN_3 | EPB41L5 | 7.36E−07 |
| PIMN_3 | RP11-14N7.2 | 7.38E−07 |
| PIMN_3 | P2RY6 | 9.09E−07 |
| PIMN_3 | LINC00284 | 9.09E−07 |
| PIMN_3 | HCN1 | 9.09E−07 |
| PIMN_3 | PCP4 | 1.07E−06 |
| PIMN_3 | SAMD5 | 1.89E−06 |
| PIMN_3 | DPYD | 2.18E−06 |
| PIMN_3 | FRMPD1 | 2.30E−06 |
| PIMN_3 | RP11-430H10.4 | 2.64E−06 |
| PIMN_3 | ASL | 2.64E−06 |
| PIMN_3 | NEGR1 | 2.89E−06 |
| PIMN_3 | SIPA1L2 | 3.91E−06 |
| PIMN_3 | DGKB | 5.44E−06 |
| PIMN_3 | GNG8 | 5.56E−06 |
| PIMN_3 | KCNJ5 | 5.68E−06 |
| PIMN_3 | KCNQ5 | 6.32E−06 |
| PIMN_3 | PHACTR3 | 9.17E−06 |
| PIMN_3 | RP11-257I14.1 | 1.16E−05 |
| PIMN_3 | NCALD | 1.32E−05 |
| PIMN_3 | SERTM1 | 1.34E−05 |
| PIMN_3 | P2RY14 | 1.46E−05 |
| PIMN_3 | TAGLN3 | 1.46E−05 |
| PIMN_3 | PDE8B | 1.59E−05 |
| PIMN_3 | PCDH9-AS2 | 1.95E−05 |
| PIMN_3 | PLEKHA6 | 1.97E−05 |
| PIMN_3 | CAMK4 | 2.11E−05 |
| PIMN_3 | HMCN2 | 2.37E−05 |
| PIMN_3 | CTD-2215E18.1 | 2.48E−05 |
| PIMN_3 | SRGAP1 | 2.84E−05 |
| PIMN_3 | GREB1L | 2.90E−05 |
| PIMN_3 | PRKD1 | 2.97E−05 |
| PIMN_3 | FHIT | 2.97E−05 |
| PIMN_3 | CACNA1C | 2.97E−05 |
| PIMN_3 | KCNC2 | 3.39E−05 |
| PIMN_3 | UCN3 | 3.43E−05 |
| PIMN_3 | RFXAP | 3.99E−05 |
| PIMN_3 | ENC1 | 4.72E−05 |
| PIMN_3 | LEPREL1 | 4.97E−05 |
| PIMN_3 | CTC-499J9.1 | 5.26E−05 |
| PIMN_3 | MYO5A | 5.58E−05 |
| PIMN_3 | RP11-307P5.1 | 5.74E−05 |
| PIMN_3 | IL12A | 6.49E−05 |
| PIMN_3 | UCP2 | 9.12E−05 |
| PIMN_3 | PROK2 | 1.64E−04 |
| PIMN_3 | HYI | 3.01E−04 |
| PIMN_3 | RP11-451M19.3 | 4.31E−04 |
| PIMN_3 | CPNE6 | 5.92E−04 |
| PIMN_3 | AKR1C2 | 8.14E−04 |
| PIMN_3 | MPP4 | 8.31E−04 |
| PIMN_3 | AJ006995.3 | 9.43E−04 |
| PIMN_3 | LINC00314 | 9.63E−04 |
| PIMN_3 | VEGFC | 1.07E−03 |
| PIMN_3 | BCAT1 | 1.41E−03 |
| PIMN_3 | PCDH19 | 1.66E−03 |
| PIMN_3 | C4orf32 | 1.72E−03 |
| PIMN_3 | TMEM237 | 1.73E−03 |
| PIMN_3 | FSTL4 | 3.67E−03 |
| PIMN_3 | DLGAP1-AS4 | 6.83E−03 |
| PIMN_3 | ANKRD2 | 7.20E−03 |
| PIMN_3 | GPR42 | 1.10E−02 |
| PIMN_3 | HOTAIRM1 | 1.14E−02 |
| PIMN_3 | FAM162B | 1.21E−02 |
| PIMN_3 | LINC00113 | 1.39E−02 |
| PIMN_3 | NEFM | 1.40E−02 |
| PIMN_3 | RP11-19O2.1 | 1.49E−02 |
| PIMN_3 | RP11-103C16.2 | 1.69E−02 |
| PIMN_3 | AC079154.1 | 1.94E−02 |
| PIMN_3 | GP1BA | 2.14E−02 |
| PIMN_3 | FFAR3 | 2.28E−02 |
| PIMN_3 | FXYD7 | 2.63E−02 |
| PIMN_3 | PLK3 | 2.63E−02 |
| PIMN_3 | GAS6-AS1 | 2.63E−02 |
| PIMN_3 | EME2 | 2.81E−02 |
| PIMN_3 | RP1-257I9.2 | 3.11E−02 |
| PIMN_3 | ZNF654 | 3.51E−02 |
| PIMN_3 | CTD-3051D23.4 | 3.51E−02 |
| PIMN_3 | TP53AIP1 | 3.84E−02 |
| PIMN_3 | RASEF | 3.86E−02 |
| PIMN_3 | RP11-186N15.3 | 3.98E−02 |
| PIMN_3 | AC007743.1 | 4.20E−02 |
| PIMN_3 | KCNF1 | 4.49E−02 |
| PIMN_3 | TP53INP1 | 4.50E−02 |
| PIMN_3 | AP1G2 | 4.59E−02 |
| PIMN_3 | MAFK | 4.79E−02 |
| PIMN_3 | RELL2 | 4.81E−02 |
| PIMN_3 | AC004692.4 | 4.87E−02 |
| PIMN_4 | NOS1 | 2.11E−21 |
| PIMN_4 | TMTC2 | 2.18E−18 |
| PIMN_4 | TANC1 | 2.37E−17 |
| PIMN_4 | TPST1 | 4.47E−16 |
| PIMN_4 | DGKB | 5.46E−15 |
| PIMN_4 | ROBO1 | 9.76E−15 |
| PIMN_4 | RP11-286N3.2 | 1.20E−14 |
| PIMN_4 | ODAM | 1.51E−12 |
| PIMN_4 | RP11-318M2.2 | 9.21E−12 |
| PIMN_4 | GUCY1A2 | 7.09E−11 |
| PIMN_4 | PRKCE | 7.09E−11 |
| PIMN_4 | ST18 | 1.94E−10 |
| PIMN_4 | LDLRAD3 | 9.29E−09 |
| PIMN_4 | MAN1A1 | 2.45E−08 |
| PIMN_4 | NLGN1 | 3.10E−08 |
| PIMN_4 | ENTPD3 | 3.85E−08 |
| PIMN_4 | RP11-131L23.1 | 4.01E−08 |
| PIMN_4 | DCLK1 | 3.82E−07 |
| PIMN_4 | PGM2L1 | 6.02E−07 |
| PIMN_4 | ARHGEF28 | 6.02E−07 |
| PIMN_4 | SNTB1 | 6.51E−07 |
| PIMN_4 | PLCB4 | 7.19E−07 |
| PIMN_4 | RIC3 | 9.79E−07 |
| PIMN_4 | NTF3 | 1.21E−06 |
| PIMN_4 | DCC | 1.53E−06 |
| PIMN_4 | NHLRC3 | 2.80E−06 |
| PIMN_4 | CTC-45812.2 | 3.05E−06 |
| PIMN_4 | EPB41L3 | 3.69E−06 |
| PIMN_4 | CIT | 4.31E−06 |
| PIMN_4 | RHOB | 4.75E−06 |
| PIMN_4 | TSPAN13 | 4.75E−06 |
| PIMN_4 | AC018890.6 | 5.78E−06 |
| PIMN_4 | AC108142.1 | 5.80E−06 |
| PIMN_4 | RP11-196H14.2 | 6.12E−06 |
| PIMN_4 | MYO1B | 6.23E−06 |
| PIMN_4 | BAALC | 6.47E−06 |
| PIMN_4 | RP11-778J15.1 | 7.10E−06 |
| PIMN_4 | SLC25A1 | 7.24E−06 |
| PIMN_4 | SAMD4A | 8.63E−06 |
| PIMN_4 | PERP | 1.08E−05 |
| PIMN_4 | CACYBP | 1.13E−05 |
| PIMN_4 | KIAA1239 | 1.33E−05 |
| PIMN_4 | RP11-252A24.7 | 1.33E−05 |
| PIMN_4 | GAL | 1.45E−05 |
| PIMN_4 | CTD-2544M6.1 | 1.57E−05 |
| PIMN_4 | ACTN1 | 2.19E−05 |
| PIMN_4 | RP1-15D23.2 | 3.16E−05 |
| PIMN_4 | TCTEX1D1 | 3.28E−05 |
| PIMN_4 | QDPR | 4.47E−05 |
| PIMN_4 | PHACTR1 | 4.72E−05 |
| PIMN_4 | ASL | 5.98E−05 |
| PIMN_4 | RP11-452H21.1 | 6.05E−05 |
| PIMN_4 | HSP90B1 | 7.55E−05 |
| PIMN_4 | FAM188A | 9.20E−05 |
| PIMN_4 | SNRPE | 9.36E−05 |
| PIMN_4 | EXOC1 | 9.97E−05 |
| PIMN_4 | TRPM3 | 9.97E−05 |
| PIMN_4 | SCML4 | 1.03E−04 |
| PIMN_4 | SERINC5 | 1.03E−04 |
| PIMN_4 | CAMK2N1 | 1.03E−04 |
| PIMN_4 | G6PC3 | 1.12E−04 |
| PIMN_4 | PYURF | 1.21E−04 |
| PIMN_4 | BUB3 | 1.27E−04 |
| PIMN_4 | VIP | 1.28E−04 |
| PIMN_4 | EEF1B2 | 1.28E−04 |
| PIMN_4 | RP11-465I4.2 | 1.32E−04 |
| PIMN_4 | PDE8B | 1.36E−04 |
| PIMN_4 | SLC35A5 | 1.53E−04 |
| PIMN_4 | SMPD3 | 1.57E−04 |
| PIMN_4 | WBSCR22 | 1.57E−04 |
| PIMN_4 | SLC38A2 | 1.59E−04 |
| PIMN_4 | ID4 | 1.59E−04 |
| PIMN_4 | SPCS1 | 1.59E−04 |
| PIMN_4 | ANKRD44 | 1.59E−04 |
| PIMN_4 | DST | 1.62E−04 |
| PIMN_4 | MAGEH1 | 1.62E−04 |
| PIMN_4 | TMEM241 | 1.71E−04 |
| PIMN_4 | YBX1 | 1.98E−04 |
| PIMN_4 | MAMDC2 | 2.12E−04 |
| PIMN_4 | FAM171A2 | 2.43E−04 |
| PIMN_4 | CTNNA2 | 2.43E−04 |
| PIMN_4 | CYB561 | 2.53E−04 |
| PIMN_4 | CLASP1 | 2.54E−04 |
| PIMN_4 | JPX | 2.54E−04 |
| PIMN_4 | LSM6 | 2.56E−04 |
| PIMN_4 | C1orf233 | 2.60E−04 |
| PIMN_4 | SRGAP1 | 2.88E−04 |
| PIMN_4 | CDKN2D | 3.00E−04 |
| PIMN_4 | ABLIM2 | 3.02E−04 |
| PIMN_4 | RP11-3L8.3 | 3.14E−04 |
| PIMN_4 | PCMTD1 | 3.14E−04 |
| PIMN_4 | TMEM167A | 3.15E−04 |
| PIMN_4 | RP11-460H9.1 | 3.22E−04 |
| PIMN_4 | COPE | 3.22E−04 |
| PIMN_4 | TNS3 | 3.30E−04 |
| PIMN_4 | EIF3I | 3.30E−04 |
| PIMN_4 | LGALS3BP | 3.39E−04 |
| PIMN_4 | RP11-320M16.2 | 3.39E−04 |
| PIMN_4 | ABCA2 | 3.41E−04 |
| PIMN_4 | ZNF536 | 3.46E−04 |
| PIMN_4 | RP11-78F17.1 | 4.09E−04 |
| PIMN_4 | RP11-710C12.1 | 4.17E−04 |
| PIMN_4 | POP7 | 5.57E−04 |
| PIMN_4 | AC096772.6 | 5.87E−04 |
| PIMN_4 | NDUFB3 | 7.37E−04 |
| PIMN_4 | CUTA | 8.78E−04 |
| PIMN_4 | LINC00639 | 9.63E−04 |
| PIMN_4 | CHMP2A | 9.64E−04 |
| PIMN_4 | MARCKSL1 | 9.97E−04 |
| PIMN_4 | MFSD2A | 1.46E−03 |
| PIMN_4 | GSTO1 | 1.60E−03 |
| PIMN_4 | BMI1 | 1.94E−03 |
| PIMN_4 | ASPHD2 | 2.45E−03 |
| PIMN_4 | RAMP3 | 2.48E−03 |
| PIMN_4 | ATP2B3 | 3.09E−03 |
| PIMN_4 | UBE2D1 | 3.30E−03 |
| PIMN_4 | MTRNR2L11 | 3.50E−03 |
| PIMN_4 | TMEM60 | 3.67E−03 |
| PIMN_4 | SYNDIG1L | 3.88E−03 |
| PIMN_4 | SNN | 4.61E−03 |
| PIMN_4 | VPS4A | 4.61E−03 |
| PIMN_4 | TCEAL8 | 4.86E−03 |
| PIMN_4 | SLFN13 | 5.06E−03 |
| PIMN_4 | FUNDC1 | 5.28E−03 |
| PIMN_4 | CTD-2336O2.1 | 5.31E−03 |
| PIMN_4 | CDH5 | 5.40E−03 |
| PIMN_4 | JOSD2 | 5.40E−03 |
| PIMN_4 | TM2D2 | 5.40E−03 |
| PIMN_4 | PPP4C | 5.50E−03 |
| PIMN_4 | RP11-286N3.1 | 5.55E−03 |
| PIMN_4 | SLC25A14 | 5.55E−03 |
| PIMN_4 | HMGA1 | 5.55E−03 |
| PIMN_4 | HSPB7 | 5.66E−03 |
| PIMN_4 | CTD-2165H16.4 | 6.14E−03 |
| PIMN_4 | CAMK1G | 6.20E−03 |
| PIMN_4 | ARHGEF25 | 6.81E−03 |
| PIMN_4 | WDR74 | 7.08E−03 |
| PIMN_4 | FRG1 | 7.62E−03 |
| PIMN_4 | EPB41L4A-AS1 | 9.58E−03 |
| PIMN_4 | NUDT16 | 1.14E−02 |
| PIMN_4 | CRAT | 1.19E−02 |
| PIMN_4 | ECHS1 | 1.22E−02 |
| PIMN_4 | NHP2 | 1.29E−02 |
| PIMN_4 | PBDC1 | 1.38E−02 |
| PIMN_4 | USF2 | 1.39E−02 |
| PIMN_4 | MESP1 | 1.56E−02 |
| PIMN_4 | ATXN8OS | 1.60E−02 |
| PIMN_4 | CCDC106 | 1.62E−02 |
| PIMN_4 | CCDC23 | 1.66E−02 |
| PIMN_4 | FAM150A | 1.67E−02 |
| PIMN_4 | APOA1BP | 1.68E−02 |
| PIMN_4 | TOPORS-AS1 | 1.68E−02 |
| PIMN_4 | UBBP4 | 1.73E−02 |
| PIMN_4 | DERL1 | 1.80E−02 |
| PIMN_4 | S100A16 | 2.03E−02 |
| PIMN_4 | CKS1B | 2.09E−02 |
| PIMN_4 | DLX1 | 2.25E−02 |
| PIMN_4 | RSL24D1 | 2.34E−02 |
| PIMN_4 | CTD-2140B24.6 | 2.40E−02 |
| PIMN_4 | PCDH9-AS1 | 2.42E−02 |
| PIMN_4 | hsa-mir-1199 | 2.63E−02 |
| PIMN_4 | SDF2L1 | 2.64E−02 |
| PIMN_4 | GSPT2 | 2.64E−02 |
| PIMN_4 | FBLL1 | 2.78E−02 |
| PIMN_4 | MAL2 | 2.83E−02 |
| PIMN_4 | TMEM185A | 3.03E−02 |
| PIMN_4 | AKR7A2 | 3.07E−02 |
| PIMN_4 | LCA10 | 3.50E−02 |
| PIMN_4 | SYCP2 | 3.50E−02 |
| PIMN_4 | FAM96A | 3.52E−02 |
| PIMN_4 | C11orf71 | 3.67E−02 |
| PIMN_4 | SDCCAG3 | 3.67E−02 |
| PIMN_4 | CTD-2050E21.1 | 3.69E−02 |
| PIMN_4 | SOX2 | 3.72E−02 |
| PIMN_4 | TIMM8A | 3.89E−02 |
| PIMN_4 | MSRB1 | 4.21E−02 |
| PIMN_4 | BMP3 | 4.30E−02 |
| PIMN_4 | RASSF7 | 4.35E−02 |
| PIMN_4 | C6orf47 | 4.47E−02 |
| PIMN_4 | ZSCAN5B | 4.51E−02 |
| PIMN_4 | ZNF585B | 4.58E−02 |
| PIMN_4 | SLC41A3 | 4.64E−02 |
| PIMN_4 | FAM43B | 4.66E−02 |
| PIMN_4 | JAGN1 | 4.69E−02 |
| PIMN_4 | ART4 | 4.77E−02 |
| PIMN_5 | SAT1 | 8.59E−15 |
| PIMN_5 | PLXDC2 | 8.59E−15 |
| PIMN_5 | LINC00478 | 7.89E−14 |
| PIMN_5 | LGI4 | 6.97E−13 |
| PIMN_5 | NKAIN3 | 2.39E−10 |
| PIMN_5 | ABCA8 | 2.64E−10 |
| PIMN_5 | CDH19 | 2.64E−10 |
| PIMN_5 | SPARC | 4.71E−10 |
| PIMN_5 | ABCA6 | 7.91E−09 |
| PIMN_5 | EPB41L2 | 1.21E−08 |
| PIMN_5 | GRIK3 | 1.36E−08 |
| PIMN_5 | DCN | 2.81E−08 |
| PIMN_5 | NDRG2 | 7.96E−08 |
| PIMN_5 | PRIMA1 | 1.75E−07 |
| PIMN_5 | CRYAB | 1.84E−07 |
| PIMN_5 | C7 | 3.50E−07 |
| PIMN_5 | ZMIZ1-AS1 | 6.23E−07 |
| PIMN_5 | MGP | 1.01E−06 |
| PIMN_5 | NRXN3 | 1.17E−06 |
| PIMN_5 | RP11-466A17.1 | 1.87E−06 |
| PIMN_5 | TNXB | 1.87E−06 |
| PIMN_5 | SULF1 | 2.08E−06 |
| PIMN_5 | EIF2A | 2.08E−06 |
| PIMN_5 | NOX4 | 2.24E−06 |
| PIMN_5 | SAMHD1 | 2.44E−06 |
| PIMN_5 | RP11-696N14.1 | 2.55E−06 |
| PIMN_5 | DOCK1 | 3.34E−06 |
| PIMN_5 | RP11-242P2.1 | 4.65E−06 |
| PIMN_5 | UFSP2 | 4.65E−06 |
| PIMN_5 | QKI | 4.70E−06 |
| PIMN_5 | PLEKHG1 | 6.02E−06 |
| PIMN_5 | RUSC1 | 7.19E−06 |
| PIMN_5 | FBLN1 | 7.77E−06 |
| PIMN_5 | PELP1 | 7.77E−06 |
| PIMN_5 | NKAIN2 | 7.77E−06 |
| PIMN_5 | GPRC5A | 9.38E−06 |
| PIMN_5 | C1orf21 | 9.38E−06 |
| PIMN_5 | COL1A2 | 9.45E−06 |
| PIMN_5 | KCNMB4 | 9.45E−06 |
| PIMN_5 | PLEKHH2 | 1.12E−05 |
| PIMN_5 | RP11-457K10.1 | 1.25E−05 |
| PIMN_5 | UACA | 1.33E−05 |
| PIMN_5 | COL16A1 | 1.37E−05 |
| PIMN_5 | ANXA5 | 1.68E−05 |
| PIMN_5 | RASSF4 | 1.89E−05 |
| PIMN_5 | C4orf36 | 2.02E−05 |
| PIMN_5 | PPFIBP1 | 2.06E−05 |
| PIMN_5 | MYBL1 | 2.10E−05 |
| PIMN_5 | C1S | 2.70E−05 |
| PIMN_5 | CBX1 | 4.95E−05 |
| PIMN_5 | CRISPLD2 | 5.14E−05 |
| PIMN_5 | CASC14 | 5.15E−05 |
| PIMN_5 | WEE1 | 5.15E−05 |
| PIMN_5 | S100B | 5.35E−05 |
| PIMN_5 | GALNT15 | 5.75E−05 |
| PIMN_5 | GPR126 | 5.91E−05 |
| PIMN_5 | NKAP | 5.96E−05 |
| PIMN_5 | COL27A1 | 6.06E−05 |
| PIMN_5 | MATN2 | 6.91E−05 |
| PIMN_5 | FXYD1 | 6.91E−05 |
| PIMN_5 | WDR86 | 7.36E−05 |
| PIMN_5 | ADAMTS16 | 8.04E−05 |
| PIMN_5 | EBF2 | 8.28E−05 |
| PIMN_5 | PTGIS | 8.33E−05 |
| PIMN_5 | RP13-143G15.3 | 8.62E−05 |
| PIMN_5 | HMCN1 | 1.00E−04 |
| PIMN_5 | EHD1 | 1.00E−04 |
| PIMN_5 | RSPRY1 | 1.02E−04 |
| PIMN_5 | TOR1B | 1.06E−04 |
| PIMN_5 | AMDHD2 | 1.06E−04 |
| PIMN_5 | CYP46A1 | 1.16E−04 |
| PIMN_5 | DUSP15 | 1.58E−04 |
| PIMN_5 | RP11-689C9.1 | 1.65E−04 |
| PIMN_5 | COL21A1 | 1.74E−04 |
| PIMN_5 | COL18A1 | 1.83E−04 |
| PIMN_5 | RPS19 | 2.11E−04 |
| PIMN_5 | JUN | 2.14E−04 |
| PIMN_5 | PRAM1 | 2.20E−04 |
| PIMN_5 | POLR3A | 3.01E−04 |
| PIMN_5 | ARHGAP24 | 3.27E−04 |
| PIMN_5 | EFEMP1 | 3.27E−04 |
| PIMN_5 | RP11-87M18.2 | 3.34E−04 |
| PIMN_5 | RAB23 | 3.45E−04 |
| PIMN_5 | SLC22A3 | 3.45E−04 |
| PIMN_5 | ZBTB16 | 3.52E−04 |
| PIMN_5 | RPL7 | 3.52E−04 |
| PIMN_5 | LRRTM3 | 3.78E−04 |
| PIMN_5 | TIMP1 | 4.22E−04 |
| PIMN_5 | C9orf37 | 4.28E−04 |
| PIMN_5 | FADS2 | 4.32E−04 |
| PIMN_5 | WIF1 | 4.32E−04 |
| PIMN_5 | LRRC3B | 4.32E−04 |
| PIMN_5 | SPARCL1 | 4.45E−04 |
| PIMN_5 | RP13-143G15.4 | 4.45E−04 |
| PIMN_5 | SNX32 | 4.86E−04 |
| PIMN_5 | AC018890.6 | 5.18E−04 |
| PIMN_5 | EARS2 | 5.33E−04 |
| PIMN_5 | CFH | 5.46E−04 |
| PIMN_5 | HEYL | 5.72E−04 |
| PIMN_5 | IGFBP7 | 5.72E−04 |
| PIMN_5 | ZNF684 | 6.78E−04 |
| PIMN_5 | HAS2 | 7.01E−04 |
| PIMN_5 | ADA | 7.04E−04 |
| PIMN_5 | MYOC | 1.37E−03 |
| PIMN_5 | APOE | 1.48E−03 |
| PIMN_5 | GLUL | 2.05E−03 |
| PIMN_5 | RP11-387H17.4 | 2.28E−03 |
| PIMN_5 | FAM210B | 2.72E−03 |
| PIMN_5 | PLP1 | 3.31E−03 |
| PIMN_5 | ARHGAP33 | 3.31E−03 |
| PIMN_5 | CYR61 | 3.59E−03 |
| PIMN_5 | HEY2 | 3.68E−03 |
| PIMN_5 | CTD-2525I3.3 | 4.82E−03 |
| PIMN_5 | ENTPD2 | 4.98E−03 |
| PIMN_5 | ATP13A5 | 5.08E−03 |
| PIMN_5 | KLF2 | 5.43E−03 |
| PIMN_5 | C16orf59 | 6.19E−03 |
| PIMN_5 | VSTM2B | 6.78E−03 |
| PIMN_5 | C1orf85 | 6.91E−03 |
| PIMN_5 | LPL | 7.33E−03 |
| PIMN_5 | RP11-27M24.1 | 7.77E−03 |
| PIMN_5 | FEM1C | 8.57E−03 |
| PIMN_5 | MYBBP1A | 8.78E−03 |
| PIMN_5 | FAS | 9.08E−03 |
| PIMN_5 | C1orf213 | 1.00E−02 |
| PIMN_5 | RP11-179A10.1 | 1.06E−02 |
| PIMN_5 | ALG12 | 1.09E−02 |
| PIMN_5 | DPT | 1.17E−02 |
| PIMN_5 | SLC15A3 | 1.24E−02 |
| PIMN_5 | MXRA8 | 1.35E−02 |
| PIMN_5 | APOBEC2 | 1.35E−02 |
| PIMN_5 | PLEKHS1 | 1.35E−02 |
| PIMN_5 | GPNMB | 1.45E−02 |
| PIMN_5 | PI16 | 1.53E−02 |
| PIMN_5 | CCDC137 | 1.68E−02 |
| PIMN_5 | RP11-597D13.9 | 1.78E−02 |
| PIMN_5 | RAB39B | 1.79E−02 |
| PIMN_5 | IGFBP6 | 1.87E−02 |
| PIMN_5 | LEP | 1.92E−02 |
| PIMN_5 | NPR2 | 2.00E−02 |
| PIMN_5 | PRDM8 | 2.07E−02 |
| PIMN_5 | MEGF6 | 2.44E−02 |
| PIMN_5 | TCTE3 | 2.54E−02 |
| PIMN_5 | RP11-124N14.3 | 2.58E−02 |
| PIMN_5 | PRODH | 2.69E−02 |
| PIMN_5 | F2RL2 | 2.79E−02 |
| PIMN_5 | TCF21 | 2.79E−02 |
| PIMN_5 | FGL2 | 2.84E−02 |
| PIMN_5 | HEPN1 | 2.89E−02 |
| PIMN_5 | ARID5A | 3.00E−02 |
| PIMN_5 | DDIT4 | 3.47E−02 |
| PIMN_5 | C5orf64 | 3.48E−02 |
| PIMN_5 | ESM1 | 3.63E−02 |
| PIMN_5 | AC140912.1 | 3.68E−02 |
| PIMN_5 | ANKRD20A1 | 3.84E−02 |
| PIMN_5 | RP4-543J13.1 | 4.02E−02 |
| PIMN_5 | CFD | 4.16E−02 |
| PIMN_5 | PRKCDBP | 4.46E−02 |
| PIMN_5 | RP11-496I9.1 | 4.51E−02 |
| PIMN_5 | TNFAIP2 | 4.66E−02 |
| PIMN_5 | CHTF18 | 4.69E−02 |
| PIMN_5 | CMTM5 | 4.72E−02 |
| PIN_1 | PENK | 2.60E−42 |
| PIN_1 | LRRTM4 | 2.56E−37 |
| PIN_1 | SGCZ | 2.62E−35 |
| PIN_1 | CNTN4 | 1.76E−33 |
| PIN_1 | PLCXD3 | 1.01E−26 |
| PIN_1 | CNTN6 | 4.49E−24 |
| PIN_1 | USH1C | 4.98E−24 |
| PIN_1 | TENM2 | 4.14E−23 |
| PIN_1 | CNTN5 | 7.28E−22 |
| PIN_1 | FAM19A2 | 7.44E−22 |
| PIN_1 | LIN7A | 1.21E−21 |
| PIN_1 | CLSTN2 | 2.55E−20 |
| PIN_1 | ASIC2 | 2.55E−20 |
| PIN_1 | SNAP25 | 6.15E−18 |
| PIN_1 | ZMAT4 | 6.15E−18 |
| PIN_1 | DLC1 | 6.75E−17 |
| PIN_1 | PIEZO2 | 7.44E−16 |
| PIN_1 | VAT1L | 2.78E−15 |
| PIN_1 | CACNA1E | 1.14E−14 |
| PIN_1 | VSTM2A | 5.32E−13 |
| PIN_1 | TAC3 | 5.88E−13 |
| PIN_1 | BMPER | 6.29E−13 |
| PIN_1 | SEMA3D | 1.14E−12 |
| PIN_1 | NDST4 | 1.30E−12 |
| PIN_1 | ZNF804A | 1.62E−12 |
| PIN_1 | NEBL | 1.67E−12 |
| PIN_1 | TM4SF4 | 2.87E−12 |
| PIN_1 | PPP2R2B | 1.05E−11 |
| PIN_1 | SLC16A12 | 2.78E−11 |
| PIN_1 | CHGB | 6.83E−11 |
| PIN_1 | SEMA3E | 8.40E−11 |
| PIN_1 | CAMK2A | 1.01E−10 |
| PIN_1 | AL035610.2 | 1.64E−10 |
| PIN_1 | LINC00871 | 2.65E−10 |
| PIN_1 | RALYL | 3.05E−10 |
| PIN_1 | ASTN2 | 3.22E−10 |
| PIN_1 | PDE5A | 3.39E−10 |
| PIN_1 | DCX | 1.18E−09 |
| PIN_1 | RGS4 | 1.49E−09 |
| PIN_1 | WBSCR17 | 3.86E−09 |
| PIN_1 | NELL2 | 8.32E−09 |
| PIN_1 | NRP2 | 8.56E−09 |
| PIN_1 | KCNH7 | 1.38E−08 |
| PIN_1 | DNER | 2.33E−08 |
| PIN_1 | TRPM3 | 5.97E−08 |
| PIN_1 | SCD | 9.38E−08 |
| PIN_1 | PDZRN3 | 1.10E−07 |
| PIN_1 | FAM19A5 | 1.52E−07 |
| PIN_1 | CALCRL | 1.79E−07 |
| PIN_1 | ITGB8 | 1.79E−07 |
| PIN_1 | TMC3 | 1.79E−07 |
| PIN_1 | OPRM1 | 2.22E−07 |
| PIN_1 | DHCR24 | 2.59E−07 |
| PIN_1 | KCNQ3 | 4.14E−07 |
| PIN_1 | ZC3H15 | 4.23E−07 |
| PIN_1 | MT3 | 5.07E−07 |
| PIN_1 | AP001604.3 | 6.80E−07 |
| PIN_1 | KCNT2 | 7.39E−07 |
| PIN_1 | ST6GALNAC3 | 1.12E−06 |
| PIN_1 | GPC5 | 1.14E−06 |
| PIN_1 | LBH | 1.18E−06 |
| PIN_1 | TPD52 | 1.27E−06 |
| PIN_1 | CTB-78F1.1 | 1.30E−06 |
| PIN_1 | RP11-168O10.6 | 1.34E−06 |
| PIN_1 | TMTC1 | 1.81E−06 |
| PIN_1 | LYPD6 | 2.14E−06 |
| PIN_1 | SHISA9 | 3.01E−06 |
| PIN_1 | SNCG | 4.06E−06 |
| PIN_1 | KCTD8 | 4.17E−06 |
| PIN_1 | NEFM | 5.91E−06 |
| PIN_1 | GRP | 8.32E−06 |
| PIN_1 | CHL1 | 8.55E−06 |
| PIN_1 | OGFRL1 | 9.66E−06 |
| PIN_1 | NFATC1 | 1.19E−05 |
| PIN_1 | FABP5 | 1.19E−05 |
| PIN_1 | PRKG1 | 1.31E−05 |
| PIN_1 | RAP1GAP2 | 1.32E−05 |
| PIN_1 | FBP1 | 1.33E−05 |
| PIN_1 | LRRN1 | 1.71E−05 |
| PIN_1 | ST6GALNAC5 | 1.93E−05 |
| PIN_1 | ATRNL1 | 1.93E−05 |
| PIN_1 | PCDH15 | 2.02E−05 |
| PIN_1 | ANXA1 | 2.37E−05 |
| PIN_1 | TMX4 | 2.48E−05 |
| PIN_1 | PCP4 | 2.59E−05 |
| PIN_1 | B3GNT1 | 2.66E−05 |
| PIN_1 | AC007392.3 | 3.11E−05 |
| PIN_1 | CBLN1 | 3.13E−05 |
| PIN_1 | RP11-38P22.2 | 3.29E−05 |
| PIN_1 | EDIL3 | 3.42E−05 |
| PIN_1 | RORA | 3.42E−05 |
| PIN_1 | CTB-178M22.1 | 3.76E−05 |
| PIN_1 | GPC5-AS1 | 4.00E−05 |
| PIN_1 | ELMO1-AS1 | 4.47E−05 |
| PIN_1 | MT-CO2 | 5.28E−05 |
| PIN_1 | FRMPD4 | 5.53E−05 |
| PIN_1 | MT-ND5 | 5.53E−05 |
| PIN_1 | CACNA2D1 | 5.66E−05 |
| PIN_1 | RP11-761I4.3 | 6.51E−05 |
| PIN_1 | OVCH1-AS1 | 6.64E−05 |
| PIN_1 | NPY2R | 8.63E−05 |
| PIN_1 | FRZB | 1.01E−04 |
| PIN_1 | ADORA1 | 1.14E−04 |
| PIN_1 | MYO1A | 1.31E−04 |
| PIN_1 | AK4 | 1.39E−04 |
| PIN_1 | FBXL16 | 1.62E−04 |
| PIN_1 | LTK | 1.65E−04 |
| PIN_1 | TINCR | 2.36E−04 |
| PIN_1 | SDR16C5 | 3.75E−04 |
| PIN_1 | IMPAD1 | 5.57E−04 |
| PIN_1 | CNTN4-AS2 | 5.66E−04 |
| PIN_1 | CPNE7 | 7.79E−04 |
| PIN_1 | FLRT3 | 1.20E−03 |
| PIN_1 | RP11-31I22.2 | 2.06E−03 |
| PIN_1 | CHST2 | 2.34E−03 |
| PIN_1 | INSIG1 | 3.38E−03 |
| PIN_1 | CTC-265N9.1 | 3.44E−03 |
| PIN_1 | RP11-1002K11.1 | 5.25E−03 |
| PIN_1 | AC068831.10 | 5.60E−03 |
| PIN_1 | C6orf141 | 5.75E−03 |
| PIN_1 | PVRL3 | 5.87E−03 |
| PIN_1 | NUAK1 | 7.55E−03 |
| PIN_1 | ZNF385D-AS2 | 7.57E−03 |
| PIN_1 | POSTN | 7.67E−03 |
| PIN_1 | MSANTD4 | 8.59E−03 |
| PIN_1 | AC019100.3 | 1.23E−02 |
| PIN_1 | AMER3 | 1.33E−02 |
| PIN_1 | DPH3 | 1.39E−02 |
| PIN_1 | IGIP | 1.45E−02 |
| PIN_1 | RP11-269F21.3 | 1.49E−02 |
| PIN_1 | RP11-31I22.3 | 1.60E−02 |
| PIN_1 | C2CD4C | 1.85E−02 |
| PIN_1 | SNPH | 2.01E−02 |
| PIN_1 | CCR10 | 2.03E−02 |
| PIN_1 | KCNJ2 | 2.22E−02 |
| PIN_1 | RP11-129B22.1 | 2.27E−02 |
| PIN_1 | RP5-1121H13.3 | 2.75E−02 |
| PIN_1 | MAB21L2 | 2.92E−02 |
| PIN_1 | SIGMAR1 | 3.03E−02 |
| PIN_1 | HTRA1 | 3.21E−02 |
| PIN_1 | HPCAL4 | 3.25E−02 |
| PIN_1 | GLRA4 | 3.27E−02 |
| PIN_1 | SLC10A4 | 3.36E−02 |
| PIN_1 | CTA-299D3.8 | 3.67E−02 |
| PIN_1 | PRPS1 | 4.02E−02 |
| PIN_1 | TMEM132E | 4.29E−02 |
| PIN_1 | TOMM34 | 4.35E−02 |
| PIN_1 | SECTM1 | 4.41E−02 |
| PIN_2 | NELL1 | 2.31E−43 |
| PIN_2 | PCDH7 | 8.50E−40 |
| PIN_2 | NRG1 | 7.25E−20 |
| PIN_2 | NTNG1 | 3.83E−18 |
| PIN_2 | ENOX1 | 3.83E−18 |
| PIN_2 | KIF26B | 2.10E−17 |
| PIN_2 | SPP1 | 4.16E−17 |
| PIN_2 | P4HA3 | 2.42E−15 |
| PIN_2 | HS3ST4 | 3.76E−15 |
| PIN_2 | IQCJ-SCHIP1 | 1.19E−14 |
| PIN_2 | RP11-649G15.2 | 4.40E−14 |
| PIN_2 | PBX3 | 7.67E−13 |
| PIN_2 | ECEL1 | 2.58E−12 |
| PIN_2 | VAT1L | 3.44E−12 |
| PIN_2 | HECW1 | 6.68E−12 |
| PIN_2 | AC133680.1 | 2.43E−11 |
| PIN_2 | CNTN3 | 4.72E−11 |
| PIN_2 | STRA6 | 8.50E−11 |
| PIN_2 | TNS3 | 1.32E−10 |
| PIN_2 | SAMD3 | 1.32E−10 |
| PIN_2 | OXR1 | 2.87E−10 |
| PIN_2 | FDPS | 3.04E−10 |
| PIN_2 | NRP1 | 3.80E−10 |
| PIN_2 | TENM2 | 6.22E−10 |
| PIN_2 | XPR1 | 9.71E−10 |
| PIN_2 | GRP | 2.19E−09 |
| PIN_2 | RARB | 4.26E−09 |
| PIN_2 | GRIK1 | 4.26E−09 |
| PIN_2 | NEBL | 5.44E−09 |
| PIN_2 | GLCCI1 | 5.44E−09 |
| PIN_2 | KIAA0922 | 5.85E−09 |
| PIN_2 | FMN1 | 5.87E−09 |
| PIN_2 | AC026202.3 | 1.18E−08 |
| PIN_2 | CYTH3 | 1.79E−08 |
| PIN_2 | SOX30 | 1.97E−08 |
| PIN_2 | TRPS1 | 1.97E−08 |
| PIN_2 | RAB30 | 1.98E−08 |
| PIN_2 | CHRNA7 | 2.00E−08 |
| PIN_2 | DOCK2 | 2.29E−08 |
| PIN_2 | FRMD4A | 2.83E−08 |
| PIN_2 | RIT2 | 3.08E−08 |
| PIN_2 | SHISA9 | 4.08E−08 |
| PIN_2 | RGS7 | 4.87E−08 |
| PIN_2 | ERBB2IP | 5.19E−08 |
| PIN_2 | ADAM22 | 7.02E−08 |
| PIN_2 | CCRN4L | 8.66E−08 |
| PIN_2 | RASGEF1B | 1.10E−07 |
| PIN_2 | ZNF490 | 1.16E−07 |
| PIN_2 | ATRNL1 | 1.35E−07 |
| PIN_2 | CHST1 | 2.61E−07 |
| PIN_2 | RP11-430H10.4 | 2.74E−07 |
| PIN_2 | RP11-619J20.1 | 3.17E−07 |
| PIN_2 | SEZ6L | 4.34E−07 |
| PIN_2 | SEMA5A | 5.30E−07 |
| PIN_2 | GRK5 | 5.96E−07 |
| PIN_2 | FGF12 | 6.61E−07 |
| PIN_2 | RP5-1043L13.1 | 7.19E−07 |
| PIN_2 | ATP8A2 | 8.15E−07 |
| PIN_2 | KLHL1 | 2.17E−06 |
| PIN_2 | DLGAP1 | 2.93E−06 |
| PIN_2 | FSTL4 | 2.96E−06 |
| PIN_2 | DPP6 | 3.96E−06 |
| PIN_2 | PARVB | 4.17E−06 |
| PIN_2 | KHDRBS3 | 4.52E−06 |
| PIN_2 | GPR158 | 4.68E−06 |
| PIN_2 | OR51E1 | 5.30E−06 |
| PIN_2 | RP11-142M10.2 | 5.30E−06 |
| PIN_2 | EDIL3 | 6.96E−06 |
| PIN_2 | IFI27 | 7.56E−06 |
| PIN_2 | CHL1 | 7.56E−06 |
| PIN_2 | RP11-384F7.1 | 7.56E−06 |
| PIN_2 | CNTN4 | 7.56E−06 |
| PIN_2 | AFF3 | 7.56E−06 |
| PIN_2 | BMPR1B | 8.34E−06 |
| PIN_2 | ARL8B | 8.37E−06 |
| PIN_2 | THBS4 | 1.16E−05 |
| PIN_2 | MEIS1 | 2.02E−05 |
| PIN_2 | ZFPM2 | 2.68E−05 |
| PIN_2 | CSMD3 | 2.79E−05 |
| PIN_2 | SYNPO2 | 3.16E−05 |
| PIN_2 | HS6ST2 | 3.16E−05 |
| PIN_2 | NCAM2 | 3.26E−05 |
| PIN_2 | SLC20A2 | 3.86E−05 |
| PIN_2 | ZFHX3 | 4.10E−05 |
| PIN_2 | CALCB | 5.73E−05 |
| PIN_2 | PGM2L1 | 5.88E−05 |
| PIN_2 | LTK | 5.94E−05 |
| PIN_2 | LIPH | 5.94E−05 |
| PIN_2 | TCERG1L | 6.94E−05 |
| PIN_2 | AC012123.1 | 1.14E−04 |
| PIN_2 | MYLK | 1.38E−04 |
| PIN_2 | ERC2 | 1.45E−04 |
| PIN_2 | LRRN3 | 1.75E−04 |
| PIN_2 | CPNE8 | 1.75E−04 |
| PIN_2 | OR51E2 | 1.83E−04 |
| PIN_2 | SNAP25 | 2.05E−04 |
| PIN_2 | AC007879.5 | 2.08E−04 |
| PIN_2 | TANC2 | 2.08E−04 |
| PIN_2 | CRB1 | 2.20E−04 |
| PIN_2 | AC026150.5 | 2.33E−04 |
| PIN_2 | NEFM | 3.14E−04 |
| PIN_2 | HTR2B | 4.19E−04 |
| PIN_2 | NXPH2 | 4.27E−04 |
| PIN_2 | FAM196B | 4.75E−04 |
| PIN_2 | PRR16 | 6.65E−04 |
| PIN_2 | LINC00685 | 8.53E−04 |
| PIN_2 | TMC3 | 1.24E−03 |
| PIN_2 | TMEM200A | 1.33E−03 |
| PIN_2 | CD226 | 1.39E−03 |
| PIN_2 | APOL2 | 1.39E−03 |
| PIN_2 | EHBP1L1 | 1.68E−03 |
| PIN_2 | CALB1 | 1.77E−03 |
| PIN_2 | RP11-62I21.1 | 1.85E−03 |
| PIN_2 | EMB | 2.14E−03 |
| PIN_2 | RP11-536C10.4 | 2.54E−03 |
| PIN_2 | GPC6-AS1 | 2.70E−03 |
| PIN_2 | BTBD3 | 2.84E−03 |
| PIN_2 | KLHL14 | 3.18E−03 |
| PIN_2 | SLC35G1 | 3.76E−03 |
| PIN_2 | GPR82 | 5.99E−03 |
| PIN_2 | SMAD9 | 6.03E−03 |
| PIN_2 | RP11-1028N23.3 | 6.05E−03 |
| PIN_2 | HMGCR | 6.38E−03 |
| PIN_2 | ARL6 | 6.38E−03 |
| PIN_2 | DLGAP1-AS5 | 6.63E−03 |
| PIN_2 | RNF144A | 7.78E−03 |
| PIN_2 | CCDC74B | 9.02E−03 |
| PIN_2 | RXRG | 9.81E−03 |
| PIN_2 | ACTR5 | 9.81E−03 |
| PIN_2 | PLK2 | 9.90E−03 |
| PIN_2 | RP11-296E23.1 | 1.05E−02 |
| PIN_2 | CTD-2371O3.2 | 1.24E−02 |
| PIN_2 | CALCA | 1.41E−02 |
| PIN_2 | ACRV1 | 1.52E−02 |
| PIN_2 | CTB-178M22.1 | 2.12E−02 |
| PIN_2 | TSPAN12 | 2.36E−02 |
| PIN_2 | ITPKA | 2.38E−02 |
| PIN_2 | VEGFA | 2.46E−02 |
| PIN_2 | GALR1 | 2.85E−02 |
| PIN_2 | TBX2 | 3.05E−02 |
| PIN_2 | FSIP2 | 3.11E−02 |
| PIN_2 | MIR7-3HG | 3.56E−02 |
| PIN_2 | IDO2 | 4.56E−02 |
| PIN_2 | CYP4F35P | 4.95E−02 |
| PSN | DGKH | 4.36E−93 |
| PSN | SPOCK3 | 4.44E−89 |
| PSN | DGKG | 5.26E−75 |
| PSN | CDH6 | 1.45E−43 |
| PSN | FAM3C | 3.89E−40 |
| PSN | LUZP2 | 5.88E−38 |
| PSN | NTRK3 | 1.01E−36 |
| PSN | GUCY1A2 | 4.31E−36 |
| PSN | CBLN2 | 6.56E−36 |
| PSN | TAC1 | 1.85E−34 |
| PSN | IFI27 | 1.32E−31 |
| PSN | ASAP1 | 1.15E−30 |
| PSN | HTR3A | 4.11E−28 |
| PSN | TCF7L2 | 2.27E−26 |
| PSN | SST | 7.27E−26 |
| PSN | SLC2A13 | 1.90E−25 |
| PSN | TMSB10 | 2.82E−24 |
| PSN | TRHDE | 7.57E−24 |
| PSN | NEDD4L | 8.82E−24 |
| PSN | VGLL3 | 5.04E−23 |
| PSN | OLFM2 | 1.53E−22 |
| PSN | C6orf141 | 1.55E−22 |
| PSN | ZNF804A | 1.65E−21 |
| PSN | S100A10 | 4.09E−21 |
| PSN | SCUBE1 | 5.27E−21 |
| PSN | KCTD16 | 5.64E−21 |
| PSN | TP53I11 | 5.84E−21 |
| PSN | OLFM3 | 7.96E−21 |
| PSN | PLXNA4 | 5.62E−20 |
| PSN | NECAB2 | 1.06E−19 |
| PSN | RP11-217C7.1 | 3.32E−19 |
| PSN | DLX4 | 3.77E−19 |
| PSN | KANK4 | 8.22E−19 |
| PSN | CHL1 | 8.32E−19 |
| PSN | TBPL1 | 3.25E−18 |
| PSN | CUX2 | 1.23E−17 |
| PSN | MCTP1 | 1.49E−17 |
| PSN | C12orf75 | 1.82E−17 |
| PSN | TSPAN8 | 6.75E−17 |
| PSN | OLFM1 | 7.24E−17 |
| PSN | SCN11A | 8.01E−17 |
| PSN | RAB3B | 8.15E−17 |
| PSN | ADIRF | 2.00E−16 |
| PSN | CPNE4 | 3.18E−16 |
| PSN | HLA-C | 3.39E−16 |
| PSN | DLX3 | 8.90E−16 |
| PSN | PLSCR1 | 1.14E−15 |
| PSN | SH3BGRL3 | 1.14E−15 |
| PSN | PHOX2B | 1.14E−15 |
| PSN | DIRAS2 | 2.04E−15 |
| PSN | RP11-138I17.1 | 2.04E−15 |
| PSN | CDH9 | 3.81E−15 |
| PSN | KCNH8 | 6.62E−15 |
| PSN | RPRM | 7.73E−15 |
| PSN | ZNF804B | 1.26E−14 |
| PSN | SDC3 | 1.28E−14 |
| PSN | GUCY1A3 | 1.63E−14 |
| PSN | SLC12A7 | 2.12E−14 |
| PSN | EPDR1 | 2.34E−14 |
| PSN | STMN1 | 2.66E−14 |
| PSN | NGFR | 3.45E−14 |
| PSN | KCNAB1 | 4.84E−14 |
| PSN | UST | 6.23E−14 |
| PSN | ANO2 | 7.64E−14 |
| PSN | SYT4 | 7.65E−14 |
| PSN | STMN2 | 2.01E−13 |
| PSN | TUBA1A | 3.22E−13 |
| PSN | TMEM160 | 9.88E−13 |
| PSN | CD9 | 1.50E−12 |
| PSN | CELF3 | 1.54E−12 |
| PSN | TTC9B | 1.91E−12 |
| PSN | B2M | 2.07E−12 |
| PSN | C9orf16 | 2.17E−12 |
| PSN | SERF2 | 2.17E−12 |
| PSN | KCNB2 | 2.58E−12 |
| PSN | THRA | 3.91E−12 |
| PSN | ATOX1 | 3.95E−12 |
| PSN | CALCRL | 4.10E−12 |
| PSN | BRINP1 | 4.82E−12 |
| PSN | YWHAG | 4.82E−12 |
| PSN | LGALS1 | 6.12E−12 |
| PSN | MRPL52 | 7.53E−12 |
| PSN | GNG3 | 8.20E−12 |
| PSN | RP11-58B17.2 | 9.29E−12 |
| PSN | HLA-B | 9.46E−12 |
| PSN | C14orf132 | 1.05E−11 |
| PSN | RP11-531A24.3 | 1.34E−11 |
| PSN | RBFOX1 | 1.41E−11 |
| PSN | IFI27L2 | 1.70E−11 |
| PSN | FBXO2 | 1.79E−11 |
| PSN | LSMD1 | 1.97E−11 |
| PSN | MAP3K5 | 2.02E−11 |
| PSN | SNCG | 2.60E−11 |
| PSN | FTH1 | 2.66E−11 |
| PSN | CADM3 | 2.74E−11 |
| PSN | NEFH | 3.41E−11 |
| PSN | AKAP12 | 3.63E−11 |
| PSN | RP11-509E16.1 | 3.64E−11 |
| PSN | GUCY1B3 | 3.64E−11 |
| PSN | PEA15 | 4.92E−11 |
| PSN | SLC35D3 | 9.90E−11 |
| PSN | TBX2 | 1.79E−10 |
| PSN | NMU | 8.41E−10 |
| PSN | RP11-361F15.2 | 2.71E−08 |
| PSN | RP11-909N17.3 | 2.88E−08 |
| PSN | KCNV1 | 4.53E−08 |
| PSN | PLSCR5 | 4.82E−08 |
| PSN | MIR7-3HG | 7.50E−08 |
| PSN | DKK1 | 7.88E−08 |
| PSN | EPHB6 | 8.34E−08 |
| PSN | PDRG1 | 8.74E−08 |
| PSN | SUSD2 | 1.28E−07 |
| PSN | B3GALT6 | 3.02E−07 |
| PSN | NOG | 4.15E−07 |
| PSN | HPCA | 6.63E−07 |
| PSN | SPRY1 | 6.76E−07 |
| PSN | CNTFR | 2.39E−05 |
| PSN | HOXB7 | 3.29E−05 |
| PSN | GALR1 | 3.52E−05 |
| PSN | FZD1 | 8.29E−05 |
| PSN | RP11-247C2.2 | 1.49E−04 |
| PSN | LY6H | 2.28E−04 |
| PSN | ENHO | 2.53E−04 |
| PSN | CEACAM21 | 3.69E−04 |
| PSN | FUOM | 3.78E−04 |
| PSN | RP3-428L16.2 | 4.17E−04 |
| PSN | SIGMAR1 | 4.61E−04 |
| PSN | TMEM229A | 7.62E−04 |
| PSN | HRH3 | 7.89E−04 |
| PSN | NPY5R | 1.16E−03 |
| PSN | KCNA4 | 1.41E−03 |
| PSN | CTD-2086O20.3 | 1.57E−03 |
| PSN | CTC-338M12.5 | 1.58E−03 |
| PSN | AC011625.1 | 2.05E−03 |
| PSN | CYB5R2 | 2.29E−03 |
| PSN | MYL3 | 3.96E−03 |
| PSN | THAP1 | 4.19E−03 |
| PSN | LRRTM1 | 4.38E−03 |
| PSN | RESP18 | 4.73E−03 |
| PSN | RP11-797H7.5 | 7.96E−03 |
| PSN | OTUD6B | 8.76E−03 |
| PSN | C8orf48 | 9.15E−03 |
| PSN | CTD-2256P15.2 | 1.06E−02 |
| PSN | TMA16 | 1.10E−02 |
| PSN | CPLX3 | 1.20E−02 |
| PSN | RP5-908M14.5 | 1.20E−02 |
| PSN | ZBTB7B | 1.48E−02 |
| PSN | CTC-248O19.1 | 1.52E−02 |
| PSN | AC007126.1 | 1.52E−02 |
| PSN | AC110619.2 | 1.55E−02 |
| PSN | LINC00237 | 1.56E−02 |
| PSN | RP11-13K12.2 | 1.72E−02 |
| PSN | RNF44 | 1.78E−02 |
| PSN | CKS2 | 2.09E−02 |
| PSN | C8orf88 | 2.30E−02 |
| PSN | CRYBB3 | 2.49E−02 |
| PSN | FAM26E | 2.61E−02 |
| PSN | PCDH18 | 2.71E−02 |
| PSN | HBA1 | 2.75E−02 |
| PSN | RP11-126K1.6 | 2.81E−02 |
| PSN | MFI2-AS1 | 2.82E−02 |
| PSN | RP11-162J8.2 | 3.81E−02 |
| PSN | RP11-629G13.1 | 4.03E−02 |
| PSN | RN7SL1 | 4.09E−02 |
| PSN | RP4-561L24.3 | 4.18E−02 |
| PSN | RP11-215H22.1 | 4.37E−02 |
| PSN | AF124730.4 | 4.44E−02 |
| PSN | SHISA7 | 4.81E−02 |
| PSVN | ANO3 | 7.37E−65 |
| PSVN | LRRC4C | 1.91E−46 |
| PSVN | CALB2 | 4.74E−44 |
| PSVN | DLGAP1 | 4.04E−43 |
| PSVN | ITGBL1 | 1.03E−39 |
| PSVN | LAMA2 | 3.18E−39 |
| PSVN | KCNJ3 | 3.57E−38 |
| PSVN | FGF14 | 2.04E−37 |
| PSVN | GRIK2 | 1.02E−35 |
| PSVN | SCGN | 9.00E−34 |
| PSVN | GPR158 | 9.11E−34 |
| PSVN | DGKI | 3.15E−33 |
| PSVN | EXT1 | 4.45E−31 |
| PSVN | MGAT4C | 1.16E−30 |
| PSVN | FAM19A2 | 2.54E−29 |
| PSVN | CNTNAP2 | 3.78E−28 |
| PSVN | KCND2 | 9.85E−28 |
| PSVN | LINGO2 | 2.74E−25 |
| PSVN | KCNK2 | 2.81E−24 |
| PSVN | C1orf186 | 9.95E−23 |
| PSVN | GALNT13 | 1.62E−22 |
| PSVN | FRMD4A | 3.58E−22 |
| PSVN | KCNH8 | 6.19E−22 |
| PSVN | SYTL3 | 7.58E−22 |
| PSVN | BRINP3 | 8.29E−22 |
| PSVN | GPC5 | 4.16E−21 |
| PSVN | CNGB1 | 6.08E−21 |
| PSVN | DLC1 | 6.59E−21 |
| PSVN | IL1RAPL1 | 1.30E−20 |
| PSVN | KCNMA1 | 4.87E−20 |
| PSVN | CACNA1D | 5.43E−20 |
| PSVN | VIP | 1.22E−18 |
| PSVN | AC009227.2 | 2.94E−18 |
| PSVN | PCSK2 | 1.18E−17 |
| PSVN | GULP1 | 1.21E−17 |
| PSVN | AP1S3 | 6.09E−16 |
| PSVN | RP11-38J22.6 | 1.05E−15 |
| PSVN | ETV1 | 2.49E−15 |
| PSVN | LUZP2 | 2.78E−15 |
| PSVN | CAMK4 | 2.80E−15 |
| PSVN | LINC00693 | 9.29E−15 |
| PSVN | AHR | 1.37E−14 |
| PSVN | CPNE8 | 1.37E−14 |
| PSVN | RP11-707A18.1 | 3.89E−14 |
| PSVN | CTNNA2 | 4.43E−14 |
| PSVN | UNC5C | 7.41E−14 |
| PSVN | AKAP12 | 1.20E−13 |
| PSVN | FMN1 | 1.21E−13 |
| PSVN | EDNRA | 1.60E−13 |
| PSVN | COL5A2 | 2.70E−13 |
| PSVN | LPHN2 | 7.62E−13 |
| PSVN | SMAD9 | 1.11E−12 |
| PSVN | KIAA1456 | 1.18E−12 |
| PSVN | RP11-368L12.1 | 1.63E−12 |
| PSVN | NEGR1 | 5.52E−12 |
| PSVN | ELL2 | 5.93E−12 |
| PSVN | PTPRK | 1.25E−11 |
| PSVN | GABRB1 | 1.25E−11 |
| PSVN | GREB1L | 1.25E−11 |
| PSVN | PLXNA4 | 1.56E−11 |
| PSVN | RP11-118B18.1 | 1.60E−11 |
| PSVN | RTTN | 1.76E−11 |
| PSVN | GFRA1 | 1.76E−11 |
| PSVN | ANGPT1 | 2.02E−11 |
| PSVN | SYT10 | 4.81E−11 |
| PSVN | SUPT3H | 5.60E−11 |
| PSVN | GCGR | 6.62E−11 |
| PSVN | UNC5B | 1.29E−10 |
| PSVN | CD36 | 1.44E−10 |
| PSVN | CDH10 | 1.46E−10 |
| PSVN | NCAM2 | 1.56E−10 |
| PSVN | RP11-260M19.2 | 1.59E−10 |
| PSVN | FAM20A | 1.66E−10 |
| PSVN | PLEKHA5 | 4.19E−10 |
| PSVN | SPHKAP | 4.47E−10 |
| PSVN | GAN | 4.74E−10 |
| PSVN | THSD7A | 4.74E−10 |
| PSVN | CTD-2054N24.2 | 8.78E−10 |
| PSVN | VWDE | 1.41E−09 |
| PSVN | OSBPL6 | 1.93E−09 |
| PSVN | ARNT2 | 2.17E−09 |
| PSVN | CHRM3 | 2.38E−09 |
| PSVN | C8orf12 | 3.91E−09 |
| PSVN | ARPP21 | 4.98E−09 |
| PSVN | NOL4 | 6.26E−09 |
| PSVN | GAREM | 1.00E−08 |
| PSVN | AFF3 | 1.04E−08 |
| PSVN | SAMD12 | 1.36E−08 |
| PSVN | ATRNL1 | 1.90E−08 |
| PSVN | PCDH15 | 2.59E−08 |
| PSVN | SAV1 | 2.64E−08 |
| PSVN | RP11-547I7.1 | 4.48E−08 |
| PSVN | PRKG2 | 4.53E−08 |
| PSVN | RP5-921G16.1 | 4.55E−08 |
| PSVN | NLGN4Y | 4.79E−08 |
| PSVN | SMARCA2 | 5.15E−08 |
| PSVN | MCHR2-AS1 | 9.92E−08 |
| PSVN | PID1 | 1.15E−07 |
| PSVN | ZEB1 | 1.20E−07 |
| PSVN | GCLC | 1.22E−07 |
| PSVN | AGMO | 9.82E−07 |
| PSVN | CHDH | 4.86E−06 |
| PSVN | RP11-63C8.1 | 9.91E−06 |
| PSVN | RP11-374M1.3 | 1.30E−05 |
| PSVN | NR2F1 | 1.84E−05 |
| PSVN | SAMD11 | 2.23E−05 |
| PSVN | NPY2R | 2.58E−05 |
| PSVN | COL11A1 | 3.00E−05 |
| PSVN | BAI1 | 3.04E−05 |
| PSVN | RP11-148O21.6 | 5.17E−05 |
| PSVN | RP11-171L9.1 | 8.87E−05 |
| PSVN | RP11-154H12.3 | 8.89E−05 |
| PSVN | MCHR2 | 9.07E−05 |
| PSVN | RP11-145O15.3 | 2.27E−04 |
| PSVN | RP11-258O13.1 | 2.56E−04 |
| PSVN | CTC-255N20.1 | 4.54E−04 |
| PSVN | PGF | 4.87E−04 |
| PSVN | SSTR1 | 4.87E−04 |
| PSVN | BCL2L12 | 5.95E−04 |
| PSVN | PDLIM2 | 7.81E−04 |
| PSVN | RP5-837I24.4 | 8.33E−04 |
| PSVN | PRR16 | 8.65E−04 |
| PSVN | GTSCR1 | 1.03E−03 |
| PSVN | UNC5B-AS1 | 1.06E−03 |
| PSVN | NPY1R | 1.13E−03 |
| PSVN | SYT13 | 1.39E−03 |
| PSVN | PKHD1L1 | 1.43E−03 |
| PSVN | CTA-373H7.7 | 1.83E−03 |
| PSVN | UPP1 | 2.21E−03 |
| PSVN | FAM167A | 2.44E−03 |
| PSVN | C21orf91-OT1 | 3.81E−03 |
| PSVN | LRTM1 | 4.02E−03 |
| PSVN | SIDT1-AS1 | 4.02E−03 |
| PSVN | IVNS1ABP | 5.98E−03 |
| PSVN | RGS7BP | 6.84E−03 |
| PSVN | EPHB6 | 7.64E−03 |
| PSVN | RP1-200K18.1 | 1.95E−02 |
| PSVN | FGF12-AS1 | 2.29E−02 |
| PSVN | RP11-54O7.3 | 2.53E−02 |
| PSVN | LINC01159 | 2.61E−02 |
| PSVN | KCTD9 | 2.64E−02 |
| PSVN | CSN1S1 | 2.66E−02 |
| PSVN | SFTPB | 2.67E−02 |
| PSVN | CTD-3032J10.2 | 2.77E−02 |
| PSVN | IL13RA2 | 3.10E−02 |
| PSVN | RP11-47J17.2 | 3.15E−02 |
| PSVN | MMRN1 | 3.30E−02 |
| PSVN | TWISTNB | 3.98E−02 |
| PSVN | CNGA3 | 4.29E−02 |
| PSVN | CCDC155 | 4.29E−02 |
| PSVN | SSSCA1 | 4.29E−02 |
| PSVN | CD200R1 | 4.81E−02 |

TABLE 22

| ident | gene | padjH |
|---|---|---|
| Glia_1 | LSAMP | 7.01E−59 |
| Glia_1 | BAI3 | 1.46E−48 |
| Glia_1 | NKAIN2 | 5.22E−41 |
| Glia_1 | CTNNA3 | 4.88E−37 |
| Glia_1 | CTNND2 | 7.79E−37 |
| Glia_1 | TPD52L1 | 1.58E−36 |
| Glia_1 | ABCA8 | 1.10E−29 |
| Glia_1 | LRRTM3 | 6.34E−28 |
| Glia_1 | PPP2R2B | 2.03E−24 |
| Glia_1 | FADS2 | 3.08E−24 |
| Glia_1 | RP11-77K12.4 | 4.80E−24 |
| Glia_1 | ATP8A1 | 1.71E−20 |
| Glia_1 | HAND2-AS1 | 4.88E−20 |
| Glia_1 | RALYL | 1.62E−18 |
| Glia_1 | NRG3 | 1.97E−18 |
| Glia_1 | LRRTM4 | 5.05E−18 |
| Glia_1 | RP11-466A17.1 | 5.32E−18 |
| Glia_1 | TRDN | 8.91E−18 |
| Glia_1 | RALGPS2 | 1.51E−17 |
| Glia_1 | BCL2L14 | 4.06E−17 |
| Glia_1 | DMKN | 6.69E−17 |
| Glia_1 | RP11-532N4.2 | 2.70E−16 |
| Glia_1 | PITPNC1 | 3.78E−16 |
| Glia_1 | NKAIN3 | 8.24E−16 |
| Glia_1 | ANGPTL1 | 1.18E−15 |
| Glia_1 | RP3-525N10.2 | 1.73E−15 |
| Glia_1 | AC018890.6 | 1.77E−15 |
| Glia_1 | RP11-3L8.3 | 9.71E−15 |
| Glia_1 | CRISPLD1 | 1.03E−14 |
| Glia_1 | PLCB4 | 2.08E−14 |
| Glia_1 | SAT1 | 2.74E−14 |
| Glia_1 | LINC00478 | 4.54E−14 |
| Glia_1 | SGIP1 | 8.51E−14 |
| Glia_1 | MARCH10 | 9.82E−14 |
| Glia_1 | PPFIBP1 | 1.97E−13 |
| Glia_1 | SASH1 | 2.77E−13 |
| Glia_1 | CERS6 | 3.53E−13 |
| Glia_1 | HMCN1 | 5.45E−13 |
| Glia_1 | HAND2 | 6.35E−13 |
| Glia_1 | LSAMP-AS1 | 9.44E−13 |
| Glia_1 | MPPED2 | 1.67E−12 |
| Glia_1 | SGCD | 1.67E−12 |
| Glia_1 | MIR181A2HG | 5.75E−12 |
| Glia_1 | ZBTB7C | 2.03E−11 |
| Glia_1 | CACNA1D | 2.42E−11 |
| Glia_1 | MEG3 | 3.10E−11 |
| Glia_1 | RIMS1 | 3.90E−11 |
| Glia_1 | FRAS1 | 4.40E−11 |
| Glia_1 | SOX5 | 7.25E−11 |
| Glia_1 | SLC4A8 | 1.29E−10 |
| Glia_1 | PCDH9 | 1.39E−10 |
| Glia_1 | NGF | 2.10E−10 |
| Glia_1 | NR6A1 | 3.07E−10 |
| Glia_1 | HIBCH | 3.62E−10 |
| Glia_1 | AL592284.1 | 4.60E−10 |
| Glia_1 | RP3-510L9.1 | 4.65E−10 |
| Glia_1 | LYRM2 | 5.85E−10 |
| Glia_1 | DPP10 | 5.85E−10 |
| Glia_1 | COL11A1 | 7.54E−10 |
| Glia_1 | LTBP1 | 1.00E−09 |
| Glia_1 | TRHDE | 1.19E−09 |
| Glia_1 | HEYL | 1.69E−09 |
| Glia_1 | PRKCA | 2.13E−09 |
| Glia_1 | SYNPR | 2.34E−09 |
| Glia_1 | RP11-318K12.2 | 2.68E−09 |
| Glia_1 | DAPL1 | 2.79E−09 |
| Glia_1 | PTPRZ1 | 2.79E−09 |
| Glia_1 | LRP1B | 3.33E−09 |
| Glia_1 | PDE4B | 3.62E−09 |
| Glia_1 | WDR86 | 4.77E−09 |
| Glia_1 | FRMD3 | 4.77E−09 |
| Glia_1 | SYT10 | 5.06E−09 |
| Glia_1 | USP54 | 5.72E−09 |
| Glia_1 | PIEZO2 | 5.72E−09 |
| Glia_1 | RLBP1 | 8.85E−09 |
| Glia_1 | CD47 | 9.02E−09 |
| Glia_1 | LPHN3 | 1.08E−08 |
| Glia_1 | GABRB1 | 1.29E−08 |
| Glia_1 | NRXN3 | 1.55E−08 |
| Glia_1 | RP11-242P2.1 | 1.59E−08 |
| Glia_1 | HPGD | 1.68E−08 |
| Glia_1 | RP11-379B18.5 | 1.68E−08 |
| Glia_1 | APP | 1.68E−08 |
| Glia_1 | CXADR | 1.76E−08 |
| Glia_1 | C9orf3 | 2.13E−08 |
| Glia_1 | MOXD1 | 2.18E−08 |
| Glia_1 | PXDN | 3.84E−08 |
| Glia_1 | IGSF11 | 4.03E−08 |
| Glia_1 | ANTXR1 | 4.60E−08 |
| Glia_1 | EDIL3 | 6.10E−08 |
| Glia_1 | CTD-2269F5.1 | 6.65E−08 |
| Glia_1 | RP11-4F22.2 | 8.43E−08 |
| Glia_1 | AC037445.1 | 8.56E−08 |
| Glia_1 | CLASP2 | 1.31E−07 |
| Glia_1 | MAML2 | 2.04E−07 |
| Glia_1 | MAPK10 | 2.29E−07 |
| Glia_1 | NLGN1 | 3.30E−07 |
| Glia_1 | AC010127.3 | 3.30E−07 |
| Glia_1 | GNA14 | 4.71E−07 |
| Glia_1 | TOM1L1 | 4.83E−07 |
| Glia_1 | MYBL1 | 2.55E−06 |
| Glia_1 | ZNF680 | 9.35E−06 |
| Glia_1 | PAIP2B | 9.99E−06 |
| Glia_1 | ST3GAL1 | 9.99E−06 |
| Glia_1 | COL12A1 | 2.37E−05 |
| Glia_1 | LINC00903 | 2.47E−05 |
| Glia_1 | LPL | 3.11E−05 |
| Glia_1 | MARC1 | 7.26E−05 |
| Glia_1 | MFSD2A | 1.47E−04 |
| Glia_1 | RP4-663N10.1 | 1.73E−04 |
| Glia_1 | RP11-776H12.1 | 1.90E−04 |
| Glia_1 | WNT16 | 2.38E−04 |
| Glia_1 | SLC16A12 | 2.70E−04 |
| Glia_1 | SOX2 | 2.70E−04 |
| Glia_1 | KIAA1549L | 3.31E−04 |
| Glia_1 | CISD2 | 3.38E−04 |
| Glia_1 | AL139147.1 | 4.90E−04 |
| Glia_1 | RP11-379B18.6 | 5.89E−04 |
| Glia_1 | SERPINE2 | 6.26E−04 |
| Glia_1 | HES4 | 6.32E−04 |
| Glia_1 | IGDCC3 | 7.18E−04 |
| Glia_1 | ACAP1 | 1.09E−03 |
| Glia_1 | MYO7A | 1.26E−03 |
| Glia_1 | HMGCLL1 | 1.76E−03 |
| Glia_1 | COL9A3 | 1.84E−03 |
| Glia_1 | AC008937.2 | 2.88E−03 |
| Glia_1 | ZC3H8 | 2.89E−03 |
| Glia_1 | HEY1 | 2.91E−03 |
| Glia_1 | PHF21B | 2.91E−03 |
| Glia_1 | CCDC24 | 3.59E−03 |
| Glia_1 | SHC3 | 3.78E−03 |
| Glia_1 | PAQR6 | 4.54E−03 |
| Glia_1 | PTGDS | 8.04E−03 |
| Glia_1 | CHDH | 9.49E−03 |
| Glia_1 | R3HCC1 | 1.02E−02 |
| Glia_1 | RP11-624M8.1 | 1.13E−02 |
| Glia_1 | B3GALT1 | 1.17E−02 |
| Glia_1 | ENTPD2 | 1.22E−02 |
| Glia_1 | RP11-255H23.4 | 1.36E−02 |
| Glia_1 | STRIP2 | 1.50E−02 |
| Glia_1 | APOE | 1.52E−02 |
| Glia_1 | RHBDL3 | 1.65E−02 |
| Glia_1 | AC079117.1 | 1.85E−02 |
| Glia_1 | LCN12 | 1.99E−02 |
| Glia_1 | LINC00648 | 2.31E−02 |
| Glia_1 | RP11-481A20.11 | 2.34E−02 |
| Glia_1 | MYRFL | 2.45E−02 |
| Glia_1 | ART3 | 2.53E−02 |
| Glia_1 | GPR37 | 2.65E−02 |
| Glia_1 | AMZ2 | 2.69E−02 |
| Glia_1 | RP11-440I14.2 | 2.69E−02 |
| Glia_1 | CSRP2 | 3.02E−02 |
| Glia_1 | NKAIN4 | 3.16E−02 |
| Glia_1 | RP11-1191J2.5 | 3.22E−02 |
| Glia_1 | GSTO2 | 3.32E−02 |
| Glia_1 | TF | 3.32E−02 |
| Glia_1 | AC009542.2 | 3.55E−02 |
| Glia_1 | MRPS18B | 4.30E−02 |
| Glia_1 | GALNT3 | 4.57E−02 |
| Glia_1 | KCNE3 | 4.73E−02 |
| Glia_1 | C5orf64 | 4.74E−02 |
| Glia_2 | NRXN1 | 9.31E−131 |
| Glia_2 | XKR4 | 1.25E−95 |
| Glia_2 | ANK3 | 3.66E−70 |
| Glia_2 | SCN7A | 1.37E−67 |
| Glia_2 | FRMD4A | 4.44E−64 |
| Glia_2 | RP11-141M1.3 | 2.60E−58 |
| Glia_2 | PIRT | 9.78E−49 |
| Glia_2 | GINS3 | 2.66E−40 |
| Glia_2 | EHBP1 | 2.66E−40 |
| Glia_2 | PMP22 | 1.57E−37 |
| Glia_2 | GRIK2 | 1.36E−34 |
| Glia_2 | DLC1 | 9.43E−34 |
| Glia_2 | RP11-429O1.1 | 1.73E−33 |
| Glia_2 | RP11-142M10.2 | 1.93E−31 |
| Glia_2 | KIAA1217 | 1.02E−30 |
| Glia_2 | STARD13 | 1.94E−30 |
| Glia_2 | ARHGAP15 | 1.94E−30 |
| Glia_2 | PTPRJ | 7.46E−22 |
| Glia_2 | NTM | 3.12E−21 |
| Glia_2 | GPM6B | 1.02E−20 |
| Glia_2 | AQP7 | 2.42E−20 |
| Glia_2 | NCAM2 | 3.55E−20 |
| Glia_2 | GPR155 | 4.95E−19 |
| Glia_2 | TGFBR2 | 1.27E−18 |
| Glia_2 | TMEM71 | 8.93E−18 |
| Glia_2 | FRMD5 | 8.93E−18 |
| Glia_2 | CADM2 | 8.93E−18 |
| Glia_2 | RP11-308N19.1 | 2.99E−17 |
| Glia_2 | CBY3 | 7.87E−17 |
| Glia_2 | ARHGAP24 | 4.93E−16 |
| Glia_2 | AC092684.1 | 6.57E−16 |
| Glia_2 | PDE4DIP | 1.12E−15 |
| Glia_2 | SAV1 | 1.94E−15 |
| Glia_2 | ZNF536 | 1.94E−15 |
| Glia_2 | IL1RAPL2 | 2.22E−15 |
| Glia_2 | POLR3GL | 6.33E−15 |
| Glia_2 | FNDC3B | 6.33E−15 |
| Glia_2 | RP11-654A16.3 | 6.40E−15 |
| Glia_2 | PDZD2 | 6.40E−15 |
| Glia_2 | ACTR5 | 2.05E−14 |
| Glia_2 | SAMHD1 | 3.00E−14 |
| Glia_2 | AGAP1 | 3.00E−14 |
| Glia_2 | SCAI | 3.26E−14 |
| Glia_2 | SHISA9 | 6.03E−14 |
| Glia_2 | ANKRD33B | 9.25E−14 |
| Glia_2 | HIP1 | 1.12E−13 |
| Glia_2 | MAP4 | 3.05E−13 |
| Glia_2 | RP11-295P9.3 | 4.84E−13 |
| Glia_2 | U91319.1 | 7.42E−13 |
| Glia_2 | CADM1 | 7.78E−13 |
| Glia_2 | ALK | 1.57E−12 |
| Glia_2 | CAB39L | 1.80E−12 |
| Glia_2 | SOX6 | 2.48E−12 |
| Glia_2 | HSPG2 | 3.11E−12 |
| Glia_2 | FOXO1 | 3.59E−12 |
| Glia_2 | SPTBN1 | 4.46E−12 |
| Glia_2 | ADK | 7.69E−12 |
| Glia_2 | ADAMTSL1 | 1.15E−11 |
| Glia_2 | HEG1 | 1.20E−11 |
| Glia_2 | ST6GALNAC5 | 2.16E−11 |
| Glia_2 | LGI4 | 2.51E−11 |
| Glia_2 | B2M | 2.61E−11 |
| Glia_2 | RBMS3 | 3.51E−11 |
| Glia_2 | ATCAY | 4.28E−11 |
| Glia_2 | KLHL29 | 5.08E−11 |
| Glia_2 | MATN2 | 9.25E−11 |
| Glia_2 | SLIT2 | 9.28E−11 |
| Glia_2 | PCSK2 | 1.88E−10 |
| Glia_2 | PPP1R12A | 1.88E−10 |
| Glia_2 | KIRREL3 | 2.02E−10 |
| Glia_2 | CLIC4 | 3.07E−10 |
| Glia_2 | LGALS1 | 3.74E−10 |
| Glia_2 | ADAM19 | 5.16E−10 |
| Glia_2 | ASTN2 | 5.16E−10 |
| Glia_2 | C1orf21 | 6.06E−10 |
| Glia_2 | ABCA6 | 6.33E−10 |
| Glia_2 | IGFBP7 | 7.34E−10 |
| Glia_2 | RPL41 | 7.38E−10 |
| Glia_2 | BEAN1 | 9.36E−10 |
| Glia_2 | SDK2 | 1.25E−09 |
| Glia_2 | ALDH1A1 | 1.66E−09 |
| Glia_2 | RP11-386G21.1 | 1.67E−09 |
| Glia_2 | MAML3 | 2.10E−09 |
| Glia_2 | SYNE2 | 2.10E−09 |
| Glia_2 | IFNGR2 | 2.19E−09 |
| Glia_2 | CTDSPL | 2.66E−09 |
| Glia_2 | NEDD4L | 2.82E−09 |
| Glia_2 | ELMO1-AS1 | 2.88E−09 |
| Glia_2 | CPEB3 | 3.28E−09 |
| Glia_2 | SPARC | 3.70E−09 |
| Glia_2 | AP000462.2 | 4.67E−09 |
| Glia_2 | PTK2 | 4.76E−09 |
| Glia_2 | FIGN | 5.88E−09 |
| Glia_2 | FTH1 | 5.88E−09 |
| Glia_2 | NEGR1 | 6.83E−09 |
| Glia_2 | FAM129A | 6.83E−09 |
| Glia_2 | OSBPL9 | 9.95E−09 |
| Glia_2 | LY9 | 9.97E−09 |
| Glia_2 | GULP1 | 9.98E−09 |
| Glia_2 | DENND1B | 1.04E−08 |
| Glia_2 | MAL | 1.16E−08 |
| Glia_2 | TMEM176B | 1.17E−08 |
| Glia_2 | SH3RF3 | 1.28E−08 |
| Glia_2 | OTOGL | 1.42E−08 |
| Glia_2 | ITGB8 | 2.37E−08 |
| Glia_2 | COL5A3 | 5.39E−08 |
| Glia_2 | CTTNBP2 | 5.48E−08 |
| Glia_2 | S100B | 1.12E−07 |
| Glia_2 | LRAT | 1.23E−07 |
| Glia_2 | HSPA12A | 1.40E−07 |
| Glia_2 | AHR | 1.99E−07 |
| Glia_2 | APCDD1 | 2.30E−07 |
| Glia_2 | MX2 | 5.42E−07 |
| Glia_2 | TRABD2B | 6.22E−07 |
| Glia_2 | SLC5A7 | 1.36E−06 |
| Glia_2 | SIPA1L2 | 1.52E−06 |
| Glia_2 | COL8A1 | 3.19E−06 |
| Glia_2 | RP11-60A8.1 | 3.45E−06 |
| Glia_2 | KCNK5 | 5.53E−06 |
| Glia_2 | FXYD1 | 7.74E−06 |
| Glia_2 | KANK4 | 8.70E−06 |
| Glia_2 | L1CAM | 3.88E−05 |
| Glia_2 | RDH10 | 5.03E−05 |
| Glia_2 | CYTL1 | 6.28E−05 |
| Glia_2 | RP11-145O15.3 | 8.28E−05 |
| Glia_2 | ENDOD1 | 8.99E−05 |
| Glia_2 | LONRF1 | 1.44E−04 |
| Glia_2 | B4GALT6 | 2.38E−04 |
| Glia_2 | MYOT | 2.68E−04 |
| Glia_2 | PMP2 | 2.99E−04 |
| Glia_2 | S100A4 | 4.03E−04 |
| Glia_2 | MPZ | 4.21E−04 |
| Glia_2 | IFI44L | 4.22E−04 |
| Glia_2 | RHOC | 1.67E−03 |
| Glia_2 | MARCKS | 1.83E−03 |
| Glia_2 | OGFRL1 | 2.03E−03 |
| Glia_2 | RP11-434H14.1 | 4.17E−03 |
| Glia_2 | MTRNR2L10 | 4.38E−03 |
| Glia_2 | RGS16 | 4.41E−03 |
| Glia_2 | DDX60L | 4.46E−03 |
| Glia_2 | LPCAT2 | 4.76E−03 |
| Glia_2 | CST3 | 5.24E−03 |
| Glia_2 | PRNP | 5.37E−03 |
| Glia_2 | TAF5L | 6.07E−03 |
| Glia_2 | PHLDA3 | 7.90E−03 |
| Glia_2 | CAPN9 | 8.39E−03 |
| Glia_2 | TMEM176A | 8.72E−03 |
| Glia_2 | FSTL3 | 9.57E−03 |
| Glia_2 | SBSPON | 1.01E−02 |
| Glia_2 | GFRA3 | 1.05E−02 |
| Glia_2 | SHE | 1.05E−02 |
| Glia_2 | MFAP3L | 1.54E−02 |
| Glia_2 | PDGFA | 1.61E−02 |
| Glia_2 | RP1-249H1.4 | 1.61E−02 |
| Glia_2 | NRN1 | 1.69E−02 |
| Glia_2 | RP5-1121H13.3 | 1.70E−02 |
| Glia_2 | MRE11A | 1.71E−02 |
| Glia_2 | GAS2L3 | 1.89E−02 |
| Glia_2 | TIMM13 | 1.91E−02 |
| Glia_2 | PMEPA1 | 1.91E−02 |
| Glia_2 | IFIT2 | 1.97E−02 |
| Glia_2 | LL22NC03-2H8.5 | 2.33E−02 |
| Glia_2 | RP11-524F11.2 | 2.48E−02 |
| Glia_2 | RP11-179A7.2 | 2.61E−02 |
| Glia_2 | RP11-381O7.3 | 2.79E−02 |
| Glia_2 | GBP1 | 3.05E−02 |
| Glia_2 | SMPDL3B | 3.23E−02 |
| Glia_2 | SOD1 | 3.41E−02 |
| Glia_2 | LGALS3BP | 3.61E−02 |
| Glia_2 | HRASLS5 | 4.33E−02 |
| Glia_3 | MYH11 | 2.14E−51 |
| Glia_3 | ACTG2 | 3.96E−33 |
| Glia_3 | SVIL | 1.72E−32 |
| Glia_3 | LPP | 1.26E−27 |
| Glia_3 | MIR145 | 1.38E−21 |
| Glia_3 | FLNA | 3.11E−21 |
| Glia_3 | RP11-123O10.4 | 6.59E−21 |
| Glia_3 | ITGA5 | 2.18E−18 |
| Glia_3 | SPEG | 9.55E−17 |
| Glia_3 | SORBS1 | 1.17E−16 |
| Glia_3 | PDLIM7 | 6.58E−16 |
| Glia_3 | GEM | 9.89E−16 |
| Glia_3 | CACNA1C | 9.89E−16 |
| Glia_3 | THRB | 3.95E−15 |
| Glia_3 | TSHZ3 | 6.86E−15 |
| Glia_3 | NEAT1 | 9.88E−15 |
| Glia_3 | RP11-413B19.2 | 9.88E−15 |
| Glia_3 | RBPMS | 1.29E−14 |
| Glia_3 | PDE4D | 1.74E−14 |
| Glia_3 | KTN1-AS1 | 1.80E−14 |
| Glia_3 | AC100830.3 | 2.34E−14 |
| Glia_3 | SYNPO2 | 3.40E−14 |
| Glia_3 | MLLT3 | 3.45E−14 |
| Glia_3 | DENND3 | 4.61E−14 |
| Glia_3 | ZFR | 6.40E−14 |
| Glia_3 | ADAM33 | 6.96E−14 |
| Glia_3 | PDZRN4 | 7.32E−14 |
| Glia_3 | ACTA2 | 8.59E−14 |
| Glia_3 | UBAC2 | 8.59E−14 |
| Glia_3 | COL4A1 | 1.11E−13 |
| Glia_3 | MLLT10 | 1.11E−13 |
| Glia_3 | NT5DC3 | 1.24E−13 |
| Glia_3 | PTCHD3P1 | 1.38E−13 |
| Glia_3 | ITGB1 | 1.62E−13 |
| Glia_3 | RP11-611D20.2 | 1.62E−13 |
| Glia_3 | PDGFC | 1.62E−13 |
| Glia_3 | ACACB | 1.70E−13 |
| Glia_3 | AC005358.3 | 2.17E−13 |
| Glia_3 | AC098617.1 | 2.43E−13 |
| Glia_3 | AP001347.6 | 2.51E−13 |
| Glia_3 | ILK | 2.64E−13 |
| Glia_3 | AF001548.5 | 3.57E−13 |
| Glia_3 | MIR143HG | 3.98E−13 |
| Glia_3 | PRUNE2 | 5.11E−13 |
| Glia_3 | CCDC57 | 5.82E−13 |
| Glia_3 | SIMC1 | 6.39E−13 |
| Glia_3 | TPM4 | 6.90E−13 |
| Glia_3 | PPP1R13B | 7.37E−13 |
| Glia_3 | ARHGAP6 | 8.42E−13 |
| Glia_3 | RP11-39M21.1 | 9.55E−13 |
| Glia_3 | ACTN1 | 1.07E−12 |
| Glia_3 | SEC13 | 1.07E−12 |
| Glia_3 | CAP2 | 1.22E−12 |
| Glia_3 | CMSS1 | 1.25E−12 |
| Glia_3 | CNN1 | 1.50E−12 |
| Glia_3 | GABPB2 | 1.86E−12 |
| Glia_3 | MAST2 | 2.10E−12 |
| Glia_3 | CACNB2 | 2.62E−12 |
| Glia_3 | PLEKHO1 | 2.62E−12 |
| Glia_3 | HIBADH | 2.65E−12 |
| Glia_3 | SNX9 | 2.65E−12 |
| Glia_3 | DGKH | 3.04E−12 |
| Glia_3 | ADAMTS9-AS1 | 3.17E−12 |
| Glia_3 | PPM1L | 3.28E−12 |
| Glia_3 | CDK7 | 3.33E−12 |
| Glia_3 | SUCLG2 | 3.33E−12 |
| Glia_3 | PIP5K1B | 3.52E−12 |
| Glia_3 | DDR2 | 3.52E−12 |
| Glia_3 | C22orf23 | 3.93E−12 |
| Glia_3 | TNS1 | 5.03E−12 |
| Glia_3 | NDE1 | 5.20E−12 |
| Glia_3 | SMTN | 5.87E−12 |
| Glia_3 | ARMC9 | 5.97E−12 |
| Glia_3 | SLC4A7 | 6.06E−12 |
| Glia_3 | CRYBG3 | 6.31E−12 |
| Glia_3 | PLD5 | 6.31E−12 |
| Glia_3 | IFT140 | 6.81E−12 |
| Glia_3 | RP11-544A12.4 | 8.38E−12 |
| Glia_3 | NRDE2 | 9.43E−12 |
| Glia_3 | VTI1A | 9.43E−12 |
| Glia_3 | MSRA | 1.14E−11 |
| Glia_3 | DIRC3 | 1.20E−11 |
| Glia_3 | RP11-238K6.1 | 1.31E−11 |
| Glia_3 | MON1B | 1.32E−11 |
| Glia_3 | SLC8A1 | 1.50E−11 |
| Glia_3 | CNST | 1.63E−11 |
| Glia_3 | KIF5B | 1.69E−11 |
| Glia_3 | NME9 | 1.79E−11 |
| Glia_3 | NPLOC4 | 1.79E−11 |
| Glia_3 | ABL1 | 1.84E−11 |
| Glia_3 | ME2 | 1.88E−11 |
| Glia_3 | PRKG1 | 1.93E−11 |
| Glia_3 | GRAMD1A | 2.11E−11 |
| Glia_3 | DMD | 2.12E−11 |
| Glia_3 | DSCAM | 2.17E−11 |
| Glia_3 | PRKAG2 | 2.26E−11 |
| Glia_3 | SULF2 | 2.42E−11 |
| Glia_3 | FMN1 | 2.42E−11 |
| Glia_3 | MEIS2 | 3.22E−11 |
| Glia_3 | TTLL11 | 3.29E−11 |
| Glia_3 | RP11-521M14.2 | 1.77E−05 |
| Glia_3 | EDA2R | 1.94E−05 |
| Glia_3 | NBL1 | 1.89E−04 |
| Glia_3 | FUOM | 3.01E−04 |
| Glia_3 | PRSS50 | 3.02E−04 |
| Glia_3 | ANKRD9 | 7.08E−04 |
| Glia_3 | AP000345.2 | 7.10E−04 |
| Glia_3 | NPTX1 | 7.71E−04 |
| Glia_3 | ZNF182 | 7.81E−04 |
| Glia_3 | RP11-789C1.1 | 1.20E−03 |
| Glia_3 | C17orf77 | 1.67E−03 |
| Glia_3 | CETP | 1.77E−03 |
| Glia_3 | RP11-375H17.1 | 1.95E−03 |
| Glia_3 | CRYBB1 | 2.00E−03 |
| Glia_3 | MUCL1 | 2.14E−03 |
| Glia_3 | RP11-214L13.1 | 2.33E−03 |
| Glia_3 | RP11-818C3.1 | 2.53E−03 |
| Glia_3 | RP11-706C16.7 | 2.75E−03 |
| Glia_3 | RP4-662A9.2 | 2.76E−03 |
| Glia_3 | RAE1 | 2.81E−03 |
| Glia_3 | TEKT2 | 3.27E−03 |
| Glia_3 | RP11-956E11.1 | 3.42E−03 |
| Glia_3 | AMELY | 3.55E−03 |
| Glia_3 | NNAT | 3.94E−03 |
| Glia_3 | TMEM201 | 4.27E−03 |
| Glia_3 | RP13-297E16.5 | 4.54E−03 |
| Glia_3 | SERPINB5 | 4.58E−03 |
| Glia_3 | AC114730.7 | 4.60E−03 |
| Glia_3 | CTD-2184D3.7 | 5.00E−03 |
| Glia_3 | RP11-662M24.1 | 5.09E−03 |
| Glia_3 | RP11-736G13.1 | 5.19E−03 |
| Glia_3 | ERCC6 | 5.21E−03 |
| Glia_3 | CTD-2024I7.13 | 5.35E−03 |
| Glia_3 | RP11-338E21.3 | 5.45E−03 |
| Glia_3 | CTD-3234P18.2 | 5.88E−03 |
| Glia_3 | ZNF324B | 5.88E−03 |
| Glia_3 | RP11-10J21.5 | 5.97E−03 |
| Glia_3 | RP11-3D4.3 | 6.03E−03 |
| Glia_3 | HTR2B | 7.34E−03 |
| Glia_3 | DNM1P35 | 7.91E−03 |
| Glia_3 | HCN3 | 7.99E−03 |
| Glia_3 | MED4-AS1 | 8.00E−03 |
| Glia_3 | RP11-77B22.2 | 9.03E−03 |
| Glia_3 | RP11-265N6.2 | 9.25E−03 |
| Glia_3 | RP11-90J7.4 | 9.71E−03 |
| Glia_3 | RP11-139K4.2 | 1.06E−02 |
| Glia_3 | C12orf77 | 1.08E−02 |
| Glia_3 | RNF44 | 1.09E−02 |
| Glia_3 | RP11-550H2.1 | 1.09E−02 |
| Glia_3 | LL09NC01-251B2.3 | 1.11E−02 |
| Glia_3 | LINC00507 | 1.14E−02 |
| Glia_3 | RP11-876F14.1 | 1.15E−02 |
| Glia_3 | RP11-1028N23.3 | 1.20E−02 |
| Glia_3 | RP11-890B15.3 | 1.22E−02 |
| Glia_3 | RP11-329J18.3 | 1.23E−02 |
| Glia_3 | RP11-930O11.2 | 1.25E−02 |
| Glia_3 | DNAJB7 | 1.26E−02 |
| Glia_3 | RP11-89N17.3 | 1.32E−02 |
| Glia_3 | CES1 | 1.47E−02 |
| Glia_3 | RP4-614C15.2 | 1.55E−02 |
| Glia_3 | E2F1 | 1.64E−02 |
| Glia_3 | IL7R | 1.68E−02 |
| Glia_3 | AC100830.5 | 1.76E−02 |
| Glia_3 | AL603965.1 | 1.80E−02 |
| Glia_3 | VSIG4 | 1.80E−02 |
| Glia_3 | ALAS2 | 1.81E−02 |
| Glia_3 | RP5-973M2.2 | 1.82E−02 |
| Glia_3 | CDH24 | 1.86E−02 |
| Glia_3 | FCER1A | 2.06E−02 |
| Glia_3 | AC112693.2 | 2.06E−02 |
| Glia_3 | FAM229A | 2.09E−02 |
| Glia_3 | A2M-AS1 | 2.21E−02 |
| Glia_3 | INTS5 | 2.41E−02 |
| Glia_3 | RP11-135D11.2 | 2.42E−02 |
| Glia_3 | FAM25B | 2.50E−02 |
| Glia_3 | RNF112 | 2.51E−02 |
| Glia_3 | RP11-415D17.4 | 2.56E−02 |
| Glia_3 | LAG3 | 2.59E−02 |
| Glia_3 | IER5 | 2.67E−02 |
| Glia_3 | HCG22 | 2.71E−02 |
| Glia_3 | RP11-44F21.2 | 2.74E−02 |
| Glia_3 | METTL18 | 3.11E−02 |
| Glia_3 | ANGPTL2 | 3.43E−02 |
| Glia_3 | RP11-691G17.1 | 3.47E−02 |
| Glia_3 | AC073236.3 | 3.55E−02 |
| Glia_3 | CTC-248O19.1 | 3.63E−02 |
| Glia_3 | C1orf162 | 3.68E−02 |
| Glia_3 | HOXD11 | 3.83E−02 |
| Glia_3 | DNM3OS | 3.84E−02 |
| Glia_3 | AC025171.1 | 3.85E−02 |
| Glia_3 | LINC00692 | 3.98E−02 |
| Glia_3 | TNFSF14 | 4.06E−02 |
| Glia_3 | RP11-421F16.3 | 4.17E−02 |
| Glia_3 | DCAF11 | 4.21E−02 |
| Glia_3 | RP11-154F14.2 | 4.25E−02 |
| Glia_3 | LINC00473 | 4.43E−02 |
| Glia_3 | ZNF501 | 4.45E−02 |
| Glia_3 | RFPL1 | 4.92E−02 |
| Glia_3 | DOK2 | 4.94E−02 |
| Glia_3 | MIR142 | 4.97E−02 |
| Glia_4 | MYH11 | 8.47E−187 |
| Glia_4 | ACTG2 | 2.24E−139 |
| Glia_4 | SVIL | 3.93E−114 |
| Glia_4 | CACNA1C | 3.06E−93 |
| Glia_4 | LPP | 1.09E−81 |
| Glia_4 | PRUNE2 | 2.68E−79 |
| Glia_4 | MIR145 | 3.37E−75 |
| Glia_4 | PDZRN4 | 1.62E−71 |
| Glia_4 | SYNPO2 | 7.73E−71 |
| Glia_4 | COL6A2 | 6.28E−70 |
| Glia_4 | PRKG1 | 1.63E−69 |
| Glia_4 | FBXO32 | 1.19E−62 |
| Glia_4 | NDE1 | 1.31E−62 |
| Glia_4 | NT5DC3 | 7.84E−61 |
| Glia_4 | TPM1 | 3.26E−59 |
| Glia_4 | RBPMS | 5.40E−57 |
| Glia_4 | SLC8A1 | 1.08E−56 |
| Glia_4 | MIR143HG | 2.98E−53 |
| Glia_4 | CCBE1 | 3.52E−53 |
| Glia_4 | TPM2 | 1.92E−48 |
| Glia_4 | SMTN | 9.51E−48 |
| Glia_4 | PDLIM7 | 4.67E−46 |
| Glia_4 | FOXP2 | 2.76E−45 |
| Glia_4 | PDE4D | 1.54E−43 |
| Glia_4 | SORBS1 | 3.25E−43 |
| Glia_4 | ACTA2 | 1.11E−42 |
| Glia_4 | PCDH7 | 9.74E−42 |
| Glia_4 | MEIS1 | 2.50E−41 |
| Glia_4 | STAB2 | 7.28E−40 |
| Glia_4 | MEIS2 | 4.19E−39 |
| Glia_4 | CACNB2 | 1.31E−38 |
| Glia_4 | MYL9 | 1.81E−38 |
| Glia_4 | RP11-611D20.2 | 2.30E−37 |
| Glia_4 | LMOD1 | 1.47E−34 |
| Glia_4 | CTD-3105H18.18 | 5.75E−34 |
| Glia_4 | ACTN1 | 7.70E−34 |
| Glia_4 | GEM | 2.34E−33 |
| Glia_4 | AC005358.3 | 2.96E−32 |
| Glia_4 | DMD | 1.05E−31 |
| Glia_4 | GPM6A | 2.75E−31 |
| Glia_4 | SLC8A1-AS1 | 2.85E−30 |
| Glia_4 | PDZRN3 | 6.94E−30 |
| Glia_4 | NEXN | 2.66E−29 |
| Glia_4 | EPHA7 | 1.37E−28 |
| Glia_4 | hsa-mir-490 | 4.06E−28 |
| Glia_4 | SEMA3A | 8.45E−28 |
| Glia_4 | ITGA5 | 1.69E−27 |
| Glia_4 | AC007392.3 | 2.46E−27 |
| Glia_4 | FLNA | 1.04E−26 |
| Glia_4 | SLMAP | 2.25E−26 |
| Glia_4 | MYLK | 2.43E−26 |
| Glia_4 | DSTN | 1.92E−25 |
| Glia_4 | AP001347.6 | 6.52E−25 |
| Glia_4 | ROR2 | 1.39E−24 |
| Glia_4 | CHRM3 | 8.16E−24 |
| Glia_4 | LINC00578 | 4.70E−23 |
| Glia_4 | MGST1 | 1.25E−22 |
| Glia_4 | AF001548.5 | 1.73E−22 |
| Glia_4 | DES | 5.96E−22 |
| Glia_4 | COL6A1 | 8.32E−22 |
| Glia_4 | RBFOX3 | 1.17E−21 |
| Glia_4 | MYL6 | 3.51E−21 |
| Glia_4 | MSRB3 | 3.63E−21 |
| Glia_4 | COL4A2 | 7.72E−21 |
| Glia_4 | NEAT1 | 1.07E−20 |
| Glia_4 | CBR4 | 4.03E−20 |
| Glia_4 | CHRM2 | 4.98E−20 |
| Glia_4 | CASKIN1 | 9.35E−20 |
| Glia_4 | CNN1 | 9.58E−20 |
| Glia_4 | ENAH | 1.12E−19 |
| Glia_4 | BTG2 | 1.88E−19 |
| Glia_4 | LDB3 | 7.18E−19 |
| Glia_4 | SOGA2 | 1.39E−18 |
| Glia_4 | MON1B | 1.74E−18 |
| Glia_4 | PNCK | 2.87E−18 |
| Glia_4 | ATP2B4 | 4.66E−18 |
| Glia_4 | COL6A3 | 5.54E−18 |
| Glia_4 | AKAP12 | 9.78E−18 |
| Glia_4 | RP11-413B19.2 | 9.78E−18 |
| Glia_4 | RP11-619J20.1 | 1.91E−17 |
| Glia_4 | NAV2 | 1.95E−17 |
| Glia_4 | PPP1R12B | 2.12E−17 |
| Glia_4 | FNBP1 | 2.94E−17 |
| Glia_4 | HIF3A | 2.94E−17 |
| Glia_4 | STT3A-AS1 | 4.87E−17 |
| Glia_4 | FHL1 | 1.29E−16 |
| Glia_4 | ARHGAP6 | 1.31E−16 |
| Glia_4 | PALLD | 1.76E−16 |
| Glia_4 | AC100830.3 | 2.04E−16 |
| Glia_4 | THRB | 2.69E−16 |
| Glia_4 | RP11-123O10.4 | 2.84E−16 |
| Glia_4 | FN1 | 3.31E−16 |
| Glia_4 | RP11-166P13.4 | 4.77E−16 |
| Glia_4 | CKB | 6.16E−16 |
| Glia_4 | PBX1 | 7.94E−16 |
| Glia_4 | LINC00842 | 2.23E−15 |
| Glia_4 | ACTB | 2.57E−15 |
| Glia_4 | NRP2 | 3.18E−15 |
| Glia_4 | ITGA7 | 3.44E−15 |
| Glia_4 | CALD1 | 4.38E−15 |
| Glia_4 | CTD-2207O23.3 | 6.29E−15 |
| Glia_4 | C20orf166-AS1 | 1.23E−14 |
| Glia_4 | FBXL22 | 2.52E−14 |
| Glia_4 | ITPKB-AS1 | 3.77E−14 |
| Glia_4 | HOXD10 | 1.48E−12 |
| Glia_4 | SLC2A4 | 5.85E−12 |
| Glia_4 | TSPAN2 | 2.24E−11 |
| Glia_4 | PCA3 | 9.94E−10 |
| Glia_4 | FENDRR | 3.11E−09 |
| Glia_4 | SLC29A1 | 2.93E−08 |
| Glia_4 | RP11-579E24.2 | 2.28E−07 |
| Glia_4 | GADD45G | 2.89E−07 |
| Glia_4 | ITGB5-AS1 | 8.24E−07 |
| Glia_4 | RP11-753H16.3 | 8.57E−07 |
| Glia_4 | BMP3 | 1.36E−06 |
| Glia_4 | CACNA1H | 1.43E−06 |
| Glia_4 | PTGS1 | 3.87E−06 |
| Glia_4 | RP11-707P20.1 | 4.05E−06 |
| Glia_4 | HOXA-AS3 | 9.41E−06 |
| Glia_4 | MRVI1 | 5.79E−05 |
| Glia_4 | CCND1 | 1.28E−04 |
| Glia_4 | ARL4D | 3.56E−04 |
| Glia_4 | ROGDI | 3.56E−04 |
| Glia_4 | KCNJ12 | 4.87E−04 |
| Glia_4 | RP11-1277A3.1 | 1.12E−03 |
| Glia_4 | NR2F2 | 1.15E−03 |
| Glia_4 | PI15 | 1.23E−03 |
| Glia_4 | BOK | 1.29E−03 |
| Glia_4 | RRP8 | 1.37E−03 |
| Glia_4 | AC073635.5 | 2.11E−03 |
| Glia_4 | RHOU | 2.59E−03 |
| Glia_4 | RP11-6O2.3 | 2.78E−03 |
| Glia_4 | Z83851.3 | 5.92E−03 |
| Glia_4 | TACR2 | 6.19E−03 |
| Glia_4 | RP11-790J24.1 | 6.32E−03 |
| Glia_4 | RP13-582O9.5 | 6.55E−03 |
| Glia_4 | GINS2 | 7.51E−03 |
| Glia_4 | RP11-753A21.1 | 9.09E−03 |
| Glia_4 | CTC-296K1.3 | 9.16E−03 |
| Glia_4 | MASP1 | 9.42E−03 |
| Glia_4 | MMP3 | 9.49E−03 |
| Glia_4 | CTC-296K1.4 | 9.67E−03 |
| Glia_4 | SLC26A10 | 1.16E−02 |
| Glia_4 | FAM186B | 1.20E−02 |
| Glia_4 | LPP-AS1 | 1.23E−02 |
| Glia_4 | LINC00339 | 1.67E−02 |
| Glia_4 | C11orf95 | 1.67E−02 |
| Glia_4 | MBNL1-AS1 | 1.72E−02 |
| Glia_4 | SGOL1 | 1.76E−02 |
| Glia_4 | RP11-158I9.5 | 1.87E−02 |
| Glia_4 | TMCO6 | 1.87E−02 |
| Glia_4 | PPP1R3C | 1.97E−02 |
| Glia_4 | POPDC3 | 2.11E−02 |
| Glia_4 | GPR183 | 2.12E−02 |
| Glia_4 | OSR1 | 2.40E−02 |
| Glia_4 | FAHD2B | 2.55E−02 |
| Glia_4 | GTPBP3 | 2.61E−02 |
| Glia_4 | CTD-2576D5.4 | 2.63E−02 |
| Glia_4 | ATP5SL | 2.68E−02 |
| Glia_4 | RP11-643A5.2 | 2.72E−02 |
| Glia_4 | RP11-515O17.3 | 2.91E−02 |
| Glia_4 | C1orf216 | 3.14E−02 |
| Glia_4 | RP11-514D23.1 | 3.77E−02 |
| Glia_4 | TBRG4 | 3.83E−02 |
| Glia_4 | RP4-800J21.3 | 3.98E−02 |
| Glia_4 | CTD-2184D3.6 | 4.38E−02 |
| Glia_4 | RP11-727A23.11 | 4.42E−02 |
| Glia_4 | CLEC10A | 4.43E−02 |
| Glia_4 | SHANK2-AS1 | 4.65E−02 |
| Glia_4 | HOXA9 | 4.73E−02 |
| Glia_4 | RP11-152F13.10 | 4.88E−02 |
| Glia_4 | SLC18A1 | 4.95E−02 |
| Glia_4 | TMEM18 | 4.97E−02 |
| Glia_5 | CTNND2 | 1.49E−39 |
| Glia_5 | BAI3 | 2.78E−38 |
| Glia_5 | LSAMP | 1.52E−22 |
| Glia_5 | CTNNA3 | 4.10E−20 |
| Glia_5 | COL11A1 | 3.02E−17 |
| Glia_5 | FADS2 | 7.06E−17 |
| Glia_5 | RIMS1 | 7.09E−17 |
| Glia_5 | LRRTM4 | 7.87E−17 |
| Glia_5 | IFI44L | 2.37E−15 |
| Glia_5 | HSPA1B | 1.31E−13 |
| Glia_5 | GPR126 | 1.31E−13 |
| Glia_5 | PPP2R2B | 1.47E−12 |
| Glia_5 | LPHN3 | 8.10E−12 |
| Glia_5 | NKAIN2 | 1.14E−11 |
| Glia_5 | ATP8A1 | 4.63E−11 |
| Glia_5 | IFI6 | 4.63E−11 |
| Glia_5 | KCNT2 | 4.91E−11 |
| Glia_5 | HMCN1 | 7.43E−11 |
| Glia_5 | TRIM9 | 7.43E−11 |
| Glia_5 | LRRTM3 | 5.73E−10 |
| Glia_5 | NKAIN3 | 3.76E−09 |
| Glia_5 | PCDH9 | 5.15E−09 |
| Glia_5 | NRG3 | 1.20E−08 |
| Glia_5 | PITPNC1 | 1.44E−08 |
| Glia_5 | RBFOX1 | 3.35E−08 |
| Glia_5 | APOE | 4.39E−08 |
| Glia_5 | GRM7 | 4.49E−08 |
| Glia_5 | LINC01057 | 5.30E−08 |
| Glia_5 | EPHA5 | 1.35E−07 |
| Glia_5 | LINC00478 | 1.82E−07 |
| Glia_5 | RALYL | 2.14E−07 |
| Glia_5 | MYBL1 | 3.28E−07 |
| Glia_5 | PCLO | 4.41E−07 |
| Glia_5 | GNA14 | 4.43E−07 |
| Glia_5 | RP11-179A16.1 | 4.43E−07 |
| Glia_5 | PXDN | 4.82E−07 |
| Glia_5 | SLC25A25 | 5.53E−07 |
| Glia_5 | MARCH10 | 5.97E−07 |
| Glia_5 | SLC4A8 | 8.73E−07 |
| Glia_5 | DDIT4 | 8.73E−07 |
| Glia_5 | RP11-466A17.1 | 1.19E−06 |
| Glia_5 | TJP1 | 1.30E−06 |
| Glia_5 | RP11-77K12.4 | 1.43E−06 |
| Glia_5 | HSPA1A | 1.51E−06 |
| Glia_5 | CHL1 | 1.73E−06 |
| Glia_5 | DPP10 | 2.10E−06 |
| Glia_5 | SYT10 | 2.13E−06 |
| Glia_5 | SGIP1 | 2.20E−06 |
| Glia_5 | CACNA1D | 2.85E−06 |
| Glia_5 | POLR2F | 2.95E−06 |
| Glia_5 | LURAP1L | 3.03E−06 |
| Glia_5 | GABRB1 | 4.90E−06 |
| Glia_5 | PTPN13 | 4.90E−06 |
| Glia_5 | MAPRE2 | 6.30E−06 |
| Glia_5 | KCNH8 | 6.62E−06 |
| Glia_5 | RASSF4 | 8.58E−06 |
| Glia_5 | NR6A1 | 1.03E−05 |
| Glia_5 | MIR146A | 1.31E−05 |
| Glia_5 | APP | 1.36E−05 |
| Glia_5 | CERS6 | 1.36E−05 |
| Glia_5 | GAP43 | 1.36E−05 |
| Glia_5 | MAPK10 | 1.53E−05 |
| Glia_5 | HES1 | 1.57E−05 |
| Glia_5 | DNM3 | 1.67E−05 |
| Glia_5 | ZNF804B | 1.71E−05 |
| Glia_5 | FAS | 1.76E−05 |
| Glia_5 | CSGALNACT1 | 2.10E−05 |
| Glia_5 | HEPN1 | 2.13E−05 |
| Glia_5 | NTNG2 | 2.44E−05 |
| Glia_5 | PTGDS | 3.03E−05 |
| Glia_5 | RP11-532N4.2 | 3.03E−05 |
| Glia_5 | RP11-122F24.1 | 3.26E−05 |
| Glia_5 | AXDND1 | 3.36E−05 |
| Glia_5 | DMC1 | 3.74E−05 |
| Glia_5 | WIPF1 | 3.77E−05 |
| Glia_5 | XAF1 | 3.77E−05 |
| Glia_5 | TPD52L1 | 3.80E−05 |
| Glia_5 | PDE11A | 3.87E−05 |
| Glia_5 | SRGAP3 | 4.43E−05 |
| Glia_5 | PHACTR1 | 4.59E−05 |
| Glia_5 | VCAN | 4.59E−05 |
| Glia_5 | COL12A1 | 4.59E−05 |
| Glia_5 | PLEKHA5 | 4.86E−05 |
| Glia_5 | CTD-2140G10.2 | 5.74E−05 |
| Glia_5 | TARSL2 | 6.03E−05 |
| Glia_5 | RP11-379B18.6 | 6.04E−05 |
| Glia_5 | PDE3A | 6.07E−05 |
| Glia_5 | CTD-2026G6.3 | 6.98E−05 |
| Glia_5 | SHC4 | 6.98E−05 |
| Glia_5 | RANBP9 | 7.06E−05 |
| Glia_5 | CD47 | 7.28E−05 |
| Glia_5 | RP11-85M11.2 | 7.62E−05 |
| Glia_5 | MOXD1 | 7.62E−05 |
| Glia_5 | RP11-379B18.5 | 7.97E−05 |
| Glia_5 | DUSP22 | 8.54E−05 |
| Glia_5 | PARP14 | 8.73E−05 |
| Glia_5 | FLT3 | 9.42E−05 |
| Glia_5 | TRHDE | 9.53E−05 |
| Glia_5 | TANC2 | 9.65E−05 |
| Glia_5 | C8orf46 | 9.65E−05 |
| Glia_5 | RP11-154D6.1 | 3.25E−04 |
| Glia_5 | GLDC | 3.94E−04 |
| Glia_5 | GFRAL | 5.26E−04 |
| Glia_5 | RP11-18B16.2 | 6.05E−04 |
| Glia_5 | ALDH8A1 | 6.91E−04 |
| Glia_5 | RP11-390B4.3 | 9.48E−04 |
| Glia_5 | NABP2 | 1.06E−03 |
| Glia_5 | TWIST1 | 1.15E−03 |
| Glia_5 | PRSS35 | 3.69E−03 |
| Glia_5 | C3orf20 | 3.74E−03 |
| Glia_5 | NREP-AS1 | 3.89E−03 |
| Glia_5 | ACPP | 3.94E−03 |
| Glia_5 | RP4-781K5.4 | 5.02E−03 |
| Glia_5 | HIST1H2BJ | 5.02E−03 |
| Glia_5 | RP11-789A21.1 | 5.38E−03 |
| Glia_5 | AC005235.1 | 5.63E−03 |
| Glia_5 | EBF3 | 5.87E−03 |
| Glia_5 | CYS1 | 5.93E−03 |
| Glia_5 | SLC23A3 | 7.51E−03 |
| Glia_5 | IGSF10 | 7.63E−03 |
| Glia_5 | ORMDL3 | 7.93E−03 |
| Glia_5 | AC096574.5 | 8.07E−03 |
| Glia_5 | MPZL3 | 9.03E−03 |
| Glia_5 | RP5-1039K5.16 | 9.45E−03 |
| Glia_5 | AC007106.1 | 1.04E−02 |
| Glia_5 | CDH17 | 1.06E−02 |
| Glia_5 | RP11-597K23.2 | 1.11E−02 |
| Glia_5 | WNT5A | 1.23E−02 |
| Glia_5 | PLA2G12B | 1.27E−02 |
| Glia_5 | IGSF1 | 1.28E−02 |
| Glia_5 | C5orf52 | 1.29E−02 |
| Glia_5 | CLEC1B | 1.30E−02 |
| Glia_5 | LINC00698 | 1.36E−02 |
| Glia_5 | DDX19B | 1.41E−02 |
| Glia_5 | SCNN1G | 1.42E−02 |
| Glia_5 | DDR1-AS1 | 1.57E−02 |
| Glia_5 | CLVS2 | 1.57E−02 |
| Glia_5 | COL9A2 | 1.62E−02 |
| Glia_5 | RP11-65D24.2 | 1.69E−02 |
| Glia_5 | RP11-159L20.2 | 1.80E−02 |
| Glia_5 | CHL1-AS1 | 1.84E−02 |
| Glia_5 | RP11-79P5.5 | 1.93E−02 |
| Glia_5 | RP11-138H11.1 | 1.96E−02 |
| Glia_5 | RP11-654C22.2 | 1.96E−02 |
| Glia_5 | RP11-1085N6.4 | 2.01E−02 |
| Glia_5 | MYH8 | 2.08E−02 |
| Glia_5 | AC002127.4 | 2.36E−02 |
| Glia_5 | CAPN14 | 2.39E−02 |
| Glia_5 | RP11-51L5.7 | 2.42E−02 |
| Glia_5 | AWAT2 | 2.43E−02 |
| Glia_5 | AC007966.1 | 2.43E−02 |
| Glia_5 | RP11-815M8.1 | 2.43E−02 |
| Glia_5 | JAKMIP1 | 2.43E−02 |
| Glia_5 | RP11-510M2.6 | 2.66E−02 |
| Glia_5 | SLC16A14 | 2.68E−02 |
| Glia_5 | NCR3LG1 | 2.84E−02 |
| Glia_5 | VN1R2 | 2.85E−02 |
| Glia_5 | C1orf189 | 2.90E−02 |
| Glia_5 | AC083864.3 | 2.92E−02 |
| Glia_5 | AC010890.1 | 2.98E−02 |
| Glia_5 | RP11-433J8.1 | 3.06E−02 |
| Glia_5 | RP11-945C19.4 | 3.09E−02 |
| Glia_5 | KIF19 | 3.15E−02 |
| Glia_5 | RP11-285M22.3 | 3.18E−02 |
| Glia_5 | TGFA | 3.29E−02 |
| Glia_5 | SLC31A2 | 3.29E−02 |
| Glia_5 | RFPL2 | 3.32E−02 |
| Glia_5 | RP11-64P14.7 | 3.34E−02 |
| Glia_5 | SPATA24 | 3.35E−02 |
| Glia_5 | HSPE1-MOB4 | 3.37E−02 |
| Glia_5 | PRDM9 | 3.38E−02 |
| Glia_5 | RP11-529K1.3 | 3.49E−02 |
| Glia_5 | RDH12 | 3.54E−02 |
| Glia_5 | KLK5 | 3.54E−02 |
| Glia_5 | LINC00160 | 3.55E−02 |
| Glia_5 | RP11-57J16.1 | 3.56E−02 |
| Glia_5 | AC006547.13 | 3.58E−02 |
| Glia_5 | RP11-523L1.2 | 3.67E−02 |
| Glia_5 | RP11-380D11.2 | 3.68E−02 |
| Glia_5 | RP11-486L19.2 | 3.72E−02 |
| Glia_5 | HMGB2 | 3.83E−02 |
| Glia_5 | RP11-197K6.1 | 3.83E−02 |
| Glia_5 | ABCC12 | 3.94E−02 |
| Glia_5 | KCNC1 | 3.95E−02 |
| Glia_5 | RP11-945A11.1 | 4.03E−02 |
| Glia_5 | RP11-10H3.1 | 4.12E−02 |
| Glia_5 | GBP6 | 4.33E−02 |
| Glia_5 | RP11-285C1.2 | 4.34E−02 |
| Glia_5 | RP11-285E9.6 | 4.52E−02 |
| Glia_5 | KRT73 | 4.56E−02 |
| Glia_5 | CTC-207P7.1 | 4.65E−02 |
| Glia_5 | RP11-47G4.2 | 4.71E−02 |
| Glia_5 | AADACL4 | 4.89E−02 |
| Glia_5 | RP11-327P2.5 | 4.99E−02 |
| Glia_6 | PID1 | 1.11E−67 |
| Glia_6 | TSHZ2 | 9.43E−60 |
| Glia_6 | RP4-678D15.1 | 9.43E−60 |
| Glia_6 | GPC6 | 1.36E−56 |
| Glia_6 | MGP | 5.20E−55 |
| Glia_6 | DCN | 1.30E−46 |
| Glia_6 | C7 | 1.06E−45 |
| Glia_6 | DPT | 4.17E−43 |
| Glia_6 | LAMA2 | 2.63E−36 |
| Glia_6 | RORA | 7.51E−33 |
| Glia_6 | EBF1 | 1.35E−30 |
| Glia_6 | SULF1 | 3.10E−28 |
| Glia_6 | ADH1B | 4.35E−28 |
| Glia_6 | LHFP | 1.52E−26 |
| Glia_6 | KCNN3 | 3.40E−26 |
| Glia_6 | DLC1 | 8.96E−26 |
| Glia_6 | PREX2 | 9.32E−26 |
| Glia_6 | RP13-143G15.4 | 7.38E−25 |
| Glia_6 | SLIT2 | 1.09E−23 |
| Glia_6 | C1orf21 | 1.75E−23 |
| Glia_6 | VIPR2 | 2.69E−21 |
| Glia_6 | RP11-385J1.2 | 1.48E−20 |
| Glia_6 | FBN1 | 2.17E−20 |
| Glia_6 | PLCB1 | 3.48E−20 |
| Glia_6 | BICC1 | 1.81E−19 |
| Glia_6 | TFPI | 2.08E−18 |
| Glia_6 | RP11-14N7.2 | 1.78E−17 |
| Glia_6 | DCLK1 | 4.55E−17 |
| Glia_6 | RP11-39M21.1 | 1.16E−16 |
| Glia_6 | RP11-648L3.2 | 2.92E−16 |
| Glia_6 | FBLN1 | 1.55E−15 |
| Glia_6 | ABCA6 | 1.61E−15 |
| Glia_6 | RP11-219B17.1 | 2.17E−15 |
| Glia_6 | NRK | 2.86E−15 |
| Glia_6 | RP11-66B24.4 | 3.36E−15 |
| Glia_6 | RP13-143G15.3 | 1.73E−14 |
| Glia_6 | PDE1A | 6.90E−14 |
| Glia_6 | AC005237.4 | 1.62E−13 |
| Glia_6 | COL5A2 | 3.44E−13 |
| Glia_6 | LAMB1 | 5.85E−13 |
| Glia_6 | NFIA | 6.24E−13 |
| Glia_6 | ABCA9 | 1.09E−12 |
| Glia_6 | AC007319.1 | 2.92E−12 |
| Glia_6 | STEAP2 | 2.92E−12 |
| Glia_6 | LUM | 1.87E−11 |
| Glia_6 | FOXO3 | 9.42E−11 |
| Glia_6 | COL6A3 | 1.26E−10 |
| Glia_6 | SVEP1 | 2.83E−10 |
| Glia_6 | PTPRG | 3.88E−10 |
| Glia_6 | NFKBIZ | 4.80E−10 |
| Glia_6 | RHOBTB3 | 4.80E−10 |
| Glia_6 | MBP | 5.74E−10 |
| Glia_6 | RBMS3 | 8.80E−10 |
| Glia_6 | LTBP4 | 9.05E−10 |
| Glia_6 | CBLB | 1.15E−09 |
| Glia_6 | LINC00478 | 1.34E−09 |
| Glia_6 | TMSB4X | 1.81E−09 |
| Glia_6 | ADAMTS1 | 2.25E−09 |
| Glia_6 | NAV3 | 2.25E−09 |
| Glia_6 | PLCL2 | 2.34E−09 |
| Glia_6 | CTA-360L10.1 | 3.45E−09 |
| Glia_6 | COL3A1 | 3.82E−09 |
| Glia_6 | BOC | 4.20E−09 |
| Glia_6 | ANXA10 | 4.61E−09 |
| Glia_6 | ELN | 4.85E−09 |
| Glia_6 | RP11-15M15.2 | 4.85E−09 |
| Glia_6 | ZBTB16 | 7.37E−09 |
| Glia_6 | DUSP1 | 7.39E−09 |
| Glia_6 | SLC9A9 | 8.45E−09 |
| Glia_6 | PIEZO2 | 9.90E−09 |
| Glia_6 | PDE7B | 1.38E−08 |
| Glia_6 | ARHGAP26-AS1 | 1.39E−08 |
| Glia_6 | PLCL1 | 1.58E−08 |
| Glia_6 | IGFBP6 | 2.57E−08 |
| Glia_6 | CITED2 | 2.91E−08 |
| Glia_6 | RP11-597D13.9 | 3.51E−08 |
| Glia_6 | MCOLN3 | 6.59E−08 |
| Glia_6 | PBX3 | 8.08E−08 |
| Glia_6 | PRR16 | 9.47E−08 |
| Glia_6 | RP11-160H12.2 | 1.19E−07 |
| Glia_6 | NEAT1 | 1.20E−07 |
| Glia_6 | GRIA4 | 1.23E−07 |
| Glia_6 | GUCY1A3 | 1.24E−07 |
| Glia_6 | KAZN | 1.88E−07 |
| Glia_6 | CCNI | 1.96E−07 |
| Glia_6 | ZFPM2 | 2.53E−07 |
| Glia_6 | PIK3R1 | 3.19E−07 |
| Glia_6 | PLXDC2 | 3.31E−07 |
| Glia_6 | PLAGL1 | 3.70E−07 |
| Glia_6 | RBMS3-AS3 | 4.28E−07 |
| Glia_6 | EPHA3 | 4.32E−07 |
| Glia_6 | PAM | 4.63E−07 |
| Glia_6 | MN1 | 4.68E−07 |
| Glia_6 | TCF21 | 5.36E−07 |
| Glia_6 | UAP1 | 5.64E−07 |
| Glia_6 | SDC2 | 6.06E−07 |
| Glia_6 | NRP1 | 6.39E−07 |
| Glia_6 | MFAP5 | 6.48E−07 |
| Glia_6 | RP11-15M15.1 | 8.47E−07 |
| Glia_6 | PDGFRA | 8.81E−07 |
| Glia_6 | AOX1 | 1.17E−06 |
| Glia_6 | PRRX1 | 2.17E−06 |
| Glia_6 | CYP4X1 | 2.53E−06 |
| Glia_6 | ADAMTS5 | 3.85E−06 |
| Glia_6 | CXCL12 | 4.52E−06 |
| Glia_6 | ALDH1A3 | 9.27E−06 |
| Glia_6 | MMP19 | 1.71E−05 |
| Glia_6 | AC012317.1 | 2.49E−05 |
| Glia_6 | CFD | 7.16E−05 |
| Glia_6 | ADCYAP1R1 | 9.79E−05 |
| Glia_6 | IGF1 | 9.99E−05 |
| Glia_6 | EFCC1 | 2.27E−04 |
| Glia_6 | SFRP2 | 2.34E−04 |
| Glia_6 | RP11-13N12.2 | 3.30E−04 |
| Glia_6 | GSTM3 | 4.13E−04 |
| Glia_6 | DIO3OS | 5.08E−04 |
| Glia_6 | MEDAG | 5.10E−04 |
| Glia_6 | ADM | 5.94E−04 |
| Glia_6 | CILP | 6.64E−04 |
| Glia_6 | PCOLCE | 1.09E−03 |
| Glia_6 | EXOC3L4 | 1.13E−03 |
| Glia_6 | TPBG | 1.24E−03 |
| Glia_6 | AC140912.1 | 1.28E−03 |
| Glia_6 | FGF10 | 1.58E−03 |
| Glia_6 | PLA2G2A | 1.89E−03 |
| Glia_6 | CD34 | 2.06E−03 |
| Glia_6 | RP11-38P22.2 | 2.79E−03 |
| Glia_6 | SPRY4 | 2.97E−03 |
| Glia_6 | CTD-2363C16.1 | 3.26E−03 |
| Glia_6 | FGF10-AS1 | 5.11E−03 |
| Glia_6 | RP11-140I24.1 | 6.60E−03 |
| Glia_6 | GUCY1B3 | 7.02E−03 |
| Glia_6 | CCL11 | 7.56E−03 |
| Glia_6 | GEMIN4 | 7.57E−03 |
| Glia_6 | CASP1 | 7.62E−03 |
| Glia_6 | KHNYN | 1.03E−02 |
| Glia_6 | FNDC1 | 1.04E−02 |
| Glia_6 | H2BFM | 1.04E−02 |
| Glia_6 | MEIS1-AS3 | 1.06E−02 |
| Glia_6 | EXOSC2 | 1.11E−02 |
| Glia_6 | PI16 | 1.11E−02 |
| Glia_6 | RP11-175K6.1 | 1.29E−02 |
| Glia_6 | BMP4 | 1.38E−02 |
| Glia_6 | TNFSF10 | 1.40E−02 |
| Glia_6 | RP11-62I21.1 | 1.46E−02 |
| Glia_6 | GPC6-AS1 | 1.52E−02 |
| Glia_6 | GADD45A | 1.52E−02 |
| Glia_6 | CYP4Z1 | 1.59E−02 |
| Glia_6 | PENK | 1.79E−02 |
| Glia_6 | PRRG3 | 1.92E−02 |
| Glia_6 | RP11-469L4.1 | 1.98E−02 |
| Glia_6 | IL32 | 2.31E−02 |
| Glia_6 | KMT2E-AS1 | 2.51E−02 |
| Glia_6 | SH2D2A | 2.55E−02 |
| Glia_6 | WISP2 | 2.60E−02 |
| Glia_6 | NPPC | 2.60E−02 |
| Glia_6 | CTB-51J22.1 | 2.66E−02 |
| Glia_6 | CH25H | 2.77E−02 |
| Glia_6 | LTF | 2.80E−02 |
| Glia_6 | P2RY1 | 3.01E−02 |
| Glia_6 | GSTM5 | 3.07E−02 |
| Glia_6 | SNAI2 | 3.24E−02 |
| Glia_6 | LY6H | 3.26E−02 |
| Glia_6 | RP11-554D13.1 | 3.31E−02 |
| Glia_6 | RP6-99M1.2 | 3.80E−02 |
| Glia_6 | AR | 3.84E−02 |
| Glia_7 | NFATC2 | 5.00E−79 |
| Glia_7 | EMP1 | 1.83E−71 |
| Glia_7 | LMNA | 9.21E−67 |
| Glia_7 | CREB5 | 5.70E−42 |
| Glia_7 | RCAN1 | 1.44E−36 |
| Glia_7 | PGM2L1 | 1.48E−30 |
| Glia_7 | ANXA1 | 1.93E−27 |
| Glia_7 | SAMD4A | 4.85E−27 |
| Glia_7 | VMP1 | 2.47E−26 |
| Glia_7 | DPYSL3 | 5.51E−26 |
| Glia_7 | MIR24-2 | 1.00E−22 |
| Glia_7 | ELL2 | 2.03E−22 |
| Glia_7 | CDH19 | 2.32E−22 |
| Glia_7 | ATP1B3 | 4.64E−22 |
| Glia_7 | PLAT | 1.46E−21 |
| Glia_7 | TNFRSF12A | 2.99E−21 |
| Glia_7 | CD44 | 8.37E−21 |
| Glia_7 | CLIC4 | 3.88E−20 |
| Glia_7 | RP11-815J21.4 | 1.89E−19 |
| Glia_7 | MYOF | 3.73E−18 |
| Glia_7 | MYO1E | 3.73E−18 |
| Glia_7 | SAT1 | 1.57E−17 |
| Glia_7 | PFKFB3 | 1.83E−17 |
| Glia_7 | CDK17 | 1.53E−16 |
| Glia_7 | RP11-414H17.5 | 4.74E−16 |
| Glia_7 | AKAP13 | 1.89E−15 |
| Glia_7 | SIK2 | 2.02E−15 |
| Glia_7 | TUBB6 | 6.33E−15 |
| Glia_7 | RP5-1042K10.10 | 9.28E−15 |
| Glia_7 | NUDT4 | 1.87E−14 |
| Glia_7 | RFX2 | 1.48E−13 |
| Glia_7 | STAT3 | 1.54E−13 |
| Glia_7 | RP3-510L9.1 | 1.86E−13 |
| Glia_7 | SIK3 | 2.26E−13 |
| Glia_7 | ZFP36 | 2.97E−13 |
| Glia_7 | AXL | 5.02E−13 |
| Glia_7 | NFATC1 | 6.12E−13 |
| Glia_7 | PTPRE | 1.13E−12 |
| Glia_7 | S100A6 | 1.36E−12 |
| Glia_7 | TPPP3 | 5.14E−12 |
| Glia_7 | ANXA2 | 6.07E−12 |
| Glia_7 | IL1RAP | 6.25E−12 |
| Glia_7 | FOSB | 6.27E−12 |
| Glia_7 | ARC | 7.76E−12 |
| Glia_7 | VCAN | 9.61E−12 |
| Glia_7 | FOSL1 | 2.52E−11 |
| Glia_7 | CCL2 | 4.06E−11 |
| Glia_7 | SGIP1 | 5.01E−11 |
| Glia_7 | CTGF | 1.40E−10 |
| Glia_7 | SEMA4A | 1.56E−10 |
| Glia_7 | MEG3 | 3.36E−10 |
| Glia_7 | S100A10 | 3.60E−10 |
| Glia_7 | NAMPT | 3.77E−10 |
| Glia_7 | RP11-2E17.1 | 4.48E−10 |
| Glia_7 | MALAT1 | 5.02E−10 |
| Glia_7 | HAS2-AS1 | 8.38E−10 |
| Glia_7 | NUP98 | 1.50E−09 |
| Glia_7 | MARCH3 | 4.00E−09 |
| Glia_7 | RP11-4F22.2 | 4.01E−09 |
| Glia_7 | RGS16 | 4.49E−09 |
| Glia_7 | RP11-123M6.2 | 5.66E−09 |
| Glia_7 | PLK3 | 8.37E−09 |
| Glia_7 | KLF6 | 8.81E−09 |
| Glia_7 | ALG13 | 9.79E−09 |
| Glia_7 | TMPRSS6 | 1.14E−08 |
| Glia_7 | CLCF1 | 1.77E−08 |
| Glia_7 | CSRNP1 | 1.77E−08 |
| Glia_7 | TACC1 | 2.16E−08 |
| Glia_7 | VIM | 2.44E−08 |
| Glia_7 | ESYT2 | 2.86E−08 |
| Glia_7 | EIF1 | 3.84E−08 |
| Glia_7 | KCTD20 | 5.23E−08 |
| Glia_7 | NR4A3 | 5.63E−08 |
| Glia_7 | PLEKHG6 | 7.01E−08 |
| Glia_7 | FAM107B | 7.07E−08 |
| Glia_7 | LEPREL1 | 7.07E−08 |
| Glia_7 | ZNRF2 | 7.80E−08 |
| Glia_7 | FOXO3 | 7.80E−08 |
| Glia_7 | SKIL | 7.81E−08 |
| Glia_7 | ARIH1 | 7.82E−08 |
| Glia_7 | NPTX2 | 9.24E−08 |
| Glia_7 | NR4A2 | 1.21E−07 |
| Glia_7 | LAMC1 | 1.23E−07 |
| Glia_7 | TNC | 1.28E−07 |
| Glia_7 | AC016831.7 | 1.54E−07 |
| Glia_7 | CTC-232P5.1 | 1.55E−07 |
| Glia_7 | GPR108 | 1.76E−07 |
| Glia_7 | S100A16 | 2.07E−07 |
| Glia_7 | NOD1 | 2.20E−07 |
| Glia_7 | ANGPTL4 | 2.59E−07 |
| Glia_7 | MIR503HG | 2.70E−07 |
| Glia_7 | ABCA8 | 3.20E−07 |
| Glia_7 | FAM129A | 3.85E−07 |
| Glia_7 | BAI3 | 4.12E−07 |
| Glia_7 | NEDD9 | 4.46E−07 |
| Glia_7 | LSAMP-AS1 | 4.99E−07 |
| Glia_7 | FAT1 | 6.21E−07 |
| Glia_7 | IFI16 | 7.77E−07 |
| Glia_7 | IQGAP2 | 8.36E−07 |
| Glia_7 | HTATIP2 | 1.21E−06 |
| Glia_7 | RP11-286E11.1 | 1.23E−06 |
| Glia_7 | RP11-689B22.2 | 1.73E−06 |
| Glia_7 | RP11-542G1.1 | 7.15E−06 |
| Glia_7 | HAS2 | 7.60E−06 |
| Glia_7 | SGMS2 | 8.33E−06 |
| Glia_7 | MIR155HG | 1.25E−05 |
| Glia_7 | IL6 | 1.25E−05 |
| Glia_7 | SERPINE1 | 3.35E−05 |
| Glia_7 | ID4 | 6.97E−05 |
| Glia_7 | MLF1 | 7.45E−05 |
| Glia_7 | F3 | 9.42E−05 |
| Glia_7 | SULT1C4 | 1.19E−04 |
| Glia_7 | SLC1A3 | 3.42E−04 |
| Glia_7 | RNF122 | 3.70E−04 |
| Glia_7 | GPR143 | 4.07E−04 |
| Glia_7 | YPEL4 | 5.87E−04 |
| Glia_7 | SPRY2 | 6.46E−04 |
| Glia_7 | ANKRD53 | 9.80E−04 |
| Glia_7 | ACHE | 1.14E−03 |
| Glia_7 | CADM4 | 2.54E−03 |
| Glia_7 | AP000688.8 | 2.70E−03 |
| Glia_7 | C12orf44 | 4.01E−03 |
| Glia_7 | SPSB1 | 4.16E−03 |
| Glia_7 | AL132709.8 | 4.82E−03 |
| Glia_7 | ODF3L1 | 5.23E−03 |
| Glia_7 | MAPK15 | 5.24E−03 |
| Glia_7 | ATP2B3 | 5.24E−03 |
| Glia_7 | RP11-4C20.3 | 9.45E−03 |
| Glia_7 | ZBTB17 | 1.00E−02 |
| Glia_7 | DBI | 1.03E−02 |
| Glia_7 | CTC-444N24.11 | 1.08E−02 |
| Glia_7 | PDLIM4 | 1.19E−02 |
| Glia_7 | KB-1732A1.1 | 1.31E−02 |
| Glia_7 | RP11-435O5.2 | 1.91E−02 |
| Glia_7 | DDX3Y | 2.01E−02 |
| Glia_7 | LINC00152 | 2.30E−02 |
| Glia_7 | TFPI2 | 2.49E−02 |
| Glia_7 | RP3-399L15.2 | 2.52E−02 |
| Glia_7 | RIBC1 | 2.76E−02 |
| Glia_7 | RP11-667K14.3 | 2.81E−02 |
| Glia_7 | RARA | 2.82E−02 |
| Glia_7 | AC133106.2 | 2.83E−02 |
| Glia_7 | KCNK3 | 3.27E−02 |
| Glia_7 | RP11-123B3.2 | 3.28E−02 |
| Glia_7 | RP11-483H20.6 | 3.34E−02 |
| Glia_7 | TMEM106A | 3.34E−02 |
| Glia_7 | LINC00205 | 3.52E−02 |
| Glia_7 | GPR56 | 3.53E−02 |
| Glia_7 | EGR3 | 4.58E−02 |
| Glia_7 | LMO2 | 4.89E−02 |
| Glia_8 | MCTP1 | 1.23E−35 |
| Glia_8 | LDB2 | 1.11E−33 |
| Glia_8 | EGFL7 | 3.45E−33 |
| Glia_8 | PTPRB | 3.17E−29 |
| Glia_8 | VWF | 2.18E−23 |
| Glia_8 | CTA-276F8.2 | 8.57E−22 |
| Glia_8 | EPAS1 | 2.53E−21 |
| Glia_8 | MECOM | 1.20E−19 |
| Glia_8 | EMP1 | 1.88E−19 |
| Glia_8 | CALCRL | 3.05E−19 |
| Glia_8 | EMCN | 5.16E−18 |
| Glia_8 | MKL2 | 5.68E−16 |
| Glia_8 | ID1 | 1.43E−15 |
| Glia_8 | ZNF385D | 3.60E−15 |
| Glia_8 | FOS | 7.78E−15 |
| Glia_8 | PIK3R3 | 1.25E−14 |
| Glia_8 | JUNB | 2.78E−14 |
| Glia_8 | MT2A | 3.58E−14 |
| Glia_8 | ELTD1 | 9.06E−14 |
| Glia_8 | ERG | 1.09E−13 |
| Glia_8 | PREX2 | 1.09E−13 |
| Glia_8 | CYYR1 | 2.93E−13 |
| Glia_8 | TMTC1 | 7.25E−13 |
| Glia_8 | ANO2 | 1.29E−12 |
| Glia_8 | SOCS3 | 2.64E−12 |
| Glia_8 | SPRY1 | 3.98E−12 |
| Glia_8 | ELMO1-AS1 | 5.84E−12 |
| Glia_8 | TSHZ2 | 8.51E−12 |
| Glia_8 | AC005237.4 | 1.06E−11 |
| Glia_8 | A2M | 1.30E−11 |
| Glia_8 | RP4-678D15.1 | 1.58E−10 |
| Glia_8 | LIFR | 1.94E−10 |
| Glia_8 | ELMO1 | 2.30E−10 |
| Glia_8 | ID3 | 3.77E−10 |
| Glia_8 | ADAMTS9 | 1.36E−09 |
| Glia_8 | MAGI1 | 1.60E−09 |
| Glia_8 | FES | 2.40E−09 |
| Glia_8 | TPO | 2.40E−09 |
| Glia_8 | RUNDC3B | 2.79E−09 |
| Glia_8 | PKP4 | 3.93E−09 |
| Glia_8 | RALGAPA2 | 3.93E−09 |
| Glia_8 | LMCD1 | 4.45E−09 |
| Glia_8 | ADCY4 | 4.55E−09 |
| Glia_8 | AL035610.2 | 1.02E−08 |
| Glia_8 | AC007319.1 | 1.02E−08 |
| Glia_8 | PALMD | 1.11E−08 |
| Glia_8 | SLC2A3 | 1.35E−08 |
| Glia_8 | SPC25 | 1.76E−08 |
| Glia_8 | SRGN | 4.41E−08 |
| Glia_8 | CXCL2 | 5.17E−08 |
| Glia_8 | TMEM100 | 5.35E−08 |
| Glia_8 | FGD4 | 5.71E−08 |
| Glia_8 | CLDN5 | 1.05E−07 |
| Glia_8 | ABLIM1 | 1.05E−07 |
| Glia_8 | TMSB10 | 1.12E−07 |
| Glia_8 | HIPK3 | 1.82E−07 |
| Glia_8 | ZFP36 | 2.01E−07 |
| Glia_8 | ST6GAL1 | 2.09E−07 |
| Glia_8 | NUAK1 | 2.26E−07 |
| Glia_8 | ADAMTS1 | 2.97E−07 |
| Glia_8 | SLCO2A1 | 4.07E−07 |
| Glia_8 | RAPGEF3 | 8.64E−07 |
| Glia_8 | ARHGAP29 | 8.64E−07 |
| Glia_8 | SERPINA5 | 1.07E−06 |
| Glia_8 | RAPGEF5 | 1.58E−06 |
| Glia_8 | PTPRM | 1.84E−06 |
| Glia_8 | PPP1R16B | 2.02E−06 |
| Glia_8 | DARC | 2.10E−06 |
| Glia_8 | HLA-E | 2.35E−06 |
| Glia_8 | ARHGAP26-AS1 | 2.62E−06 |
| Glia_8 | DUSP1 | 3.40E−06 |
| Glia_8 | RIN2 | 3.94E−06 |
| Glia_8 | CAV1 | 4.35E−06 |
| Glia_8 | SIK1 | 4.39E−06 |
| Glia_8 | FLI1 | 4.40E−06 |
| Glia_8 | THSD7A | 4.51E−06 |
| Glia_8 | SOX17 | 7.10E−06 |
| Glia_8 | CD74 | 7.30E−06 |
| Glia_8 | PRKCH | 8.47E−06 |
| Glia_8 | FLT1 | 9.02E−06 |
| Glia_8 | AC010524.4 | 1.02E−05 |
| Glia_8 | NEDD9 | 1.12E−05 |
| Glia_8 | MAP3K8 | 1.13E−05 |
| Glia_8 | UTRN | 1.25E−05 |
| Glia_8 | C4orf32 | 1.25E−05 |
| Glia_8 | ARHGAP26 | 1.25E−05 |
| Glia_8 | SDPR | 1.26E−05 |
| Glia_8 | PLEKHG1 | 1.48E−05 |
| Glia_8 | HLA-B | 1.63E−05 |
| Glia_8 | MT1E | 1.63E−05 |
| Glia_8 | TM4SF1 | 1.68E−05 |
| Glia_8 | PLXNA2 | 2.36E−05 |
| Glia_8 | RASAL2 | 2.36E−05 |
| Glia_8 | ATP8B1 | 2.43E−05 |
| Glia_8 | MT1M | 2.62E−05 |
| Glia_8 | MSN | 2.62E−05 |
| Glia_8 | ASAP1 | 2.64E−05 |
| Glia_8 | TENC1 | 2.99E−05 |
| Glia_8 | FAM110D | 3.43E−05 |
| Glia_8 | RBMS3-AS3 | 3.79E−05 |
| Glia_8 | GPIHBP1 | 4.21E−05 |
| Glia_8 | MYCT1 | 7.15E−05 |
| Glia_8 | ETS2 | 8.95E−05 |
| Glia_8 | RFTN1 | 1.44E−04 |
| Glia_8 | HYAL2 | 1.58E−04 |
| Glia_8 | ATOH8 | 2.10E−04 |
| Glia_8 | SH3BGRL2 | 2.82E−04 |
| Glia_8 | AQP1 | 5.08E−04 |
| Glia_8 | EBF3 | 5.18E−04 |
| Glia_8 | POSTN | 5.60E−04 |
| Glia_8 | SNCG | 6.49E−04 |
| Glia_8 | ECSCR | 6.53E−04 |
| Glia_8 | BCAM | 6.85E−04 |
| Glia_8 | ARHGEF15 | 7.41E−04 |
| Glia_8 | CLEC1A | 7.55E−04 |
| Glia_8 | ICAM2 | 8.46E−04 |
| Glia_8 | CD93 | 9.45E−04 |
| Glia_8 | GIMAP6 | 9.45E−04 |
| Glia_8 | MYC | 1.25E−03 |
| Glia_8 | SLC40A1 | 1.31E−03 |
| Glia_8 | RP11-818O24.3 | 1.49E−03 |
| Glia_8 | RP11-203M5.8 | 1.75E−03 |
| Glia_8 | FRAT2 | 1.93E−03 |
| Glia_8 | TMEM173 | 1.99E−03 |
| Glia_8 | THBD | 2.01E−03 |
| Glia_8 | KCNJ1 | 2.02E−03 |
| Glia_8 | LRRC32 | 2.44E−03 |
| Glia_8 | AC005550.3 | 2.44E−03 |
| Glia_8 | S1PR1 | 2.52E−03 |
| Glia_8 | EDN1 | 2.59E−03 |
| Glia_8 | CTC-484P3.3 | 2.63E−03 |
| Glia_8 | MPZL2 | 3.34E−03 |
| Glia_8 | LINC00313 | 4.19E−03 |
| Glia_8 | VEGFC | 4.71E−03 |
| Glia_8 | Z98049.1 | 5.09E−03 |
| Glia_8 | SLCO4A1 | 5.80E−03 |
| Glia_8 | ACVRL1 | 5.85E−03 |
| Glia_8 | DUSP23 | 6.61E−03 |
| Glia_8 | NOTCH4 | 6.61E−03 |
| Glia_8 | B4GALNT1 | 9.94E−03 |
| Glia_8 | NOS3 | 1.04E−02 |
| Glia_8 | MRPL28 | 1.19E−02 |
| Glia_8 | APLNR | 1.20E−02 |
| Glia_8 | ROBO4 | 1.34E−02 |
| Glia_8 | TAL1 | 1.37E−02 |
| Glia_8 | RP6-99M1.2 | 1.42E−02 |
| Glia_8 | FAM26E | 1.48E−02 |
| Glia_8 | RP11-1030E3.1 | 1.56E−02 |
| Glia_8 | FABP4 | 1.68E−02 |
| Glia_8 | RP11-64B16.5 | 1.71E−02 |
| Glia_8 | AC116614.1 | 1.79E−02 |
| Glia_8 | ESAM | 1.84E−02 |
| Glia_8 | GORASP1 | 1.89E−02 |
| Glia_8 | CLEC14A | 1.96E−02 |
| Glia_8 | HLA-DRB1 | 2.80E−02 |
| Glia_8 | MT1A | 2.94E−02 |
| Glia_8 | STC1 | 3.09E−02 |
| Glia_8 | FABP5 | 3.12E−02 |
| Glia_8 | BCL3 | 3.48E−02 |
| Glia_8 | SHANK3 | 3.55E−02 |
| Glia_8 | RP11-136H19.1 | 4.33E−02 |
| Glia_8 | GIMAP1 | 4.49E−02 |
| Glia_8 | GATA2 | 4.59E−02 |
| Glia_8 | SMAD7 | 4.76E−02 |
| Glia_8 | HCN3 | 4.76E−02 |

TABLE 23
Conserved transcriptional programs in human and mouse enteric neurons. Differentially expressed genes for major neuron
classes that are shared between human and mouse, including expression statistics for both mouse and human neurons.

| ident | gene | mouse_alpha | mouse_mean | mouse_log2fc | human_alpha | human_mean | human_log2fc |
|---|---|---|---|---|---|---|---|
| Excitatory_Motor | Abcc8 | 0.91 | 1.90 | 1.03 | 0.55 | 0.73 | 1.33 |
| Excitatory_Motor | Abtb2 | 0.72 | 0.69 | 2.02 | 0.54 | 0.93 | 1.12 |
| Excitatory_Motor | Adamtsl1 | 0.91 | 1.18 | 2.93 | 0.67 | 1.51 | 1.54 |
| Excitatory_Motor | Alk | 0.98 | 4.45 | 2.14 | 0.86 | 3.39 | 1.91 |
| Excitatory_Motor | Bnc2 | 0.98 | 4.28 | 2.30 | 0.98 | 3.64 | 1.95 |
| Excitatory_Motor | Bub3 | 0.63 | −0.18 | 0.39 | 0.59 | 1.03 | 0.47 |
| Excitatory_Motor | Calcrl | 0.83 | 1.74 | 1.05 | 0.67 | 1.74 | 1.28 |
| Excitatory_Motor | Car10 | 0.79 | 2.20 | 2.13 | 0.78 | 2.13 | 1.33 |
| Excitatory_Motor | Casz1 | 0.98 | 2.48 | 1.94 | 0.69 | 1.22 | 0.87 |
| Excitatory_Motor | Chat | 0.97 | 2.48 | 1.95 | 0.38 | −0.48 | 0.32 |
| Excitatory_Motor | Chrm2 | 0.99 | 2.96 | 0.44 | 0.50 | 1.56 | 1.91 |
| Excitatory_Motor | Cnr1 | 0.87 | 2.77 | 0.68 | 0.65 | 1.37 | 0.78 |
| Excitatory_Motor | Colq | 0.76 | 0.82 | 2.30 | 0.69 | 1.80 | 1.75 |
| Excitatory_Motor | Cpne8 | 0.99 | 2.47 | 1.32 | 0.65 | 1.42 | 0.54 |
| Excitatory_Motor | Cradd | 0.64 | −0.40 | 0.52 | 0.62 | 0.88 | 0.24 |
| Excitatory_Motor | Dlgap2 | 1.00 | 3.82 | 1.88 | 0.54 | 0.55 | 1.34 |
| Excitatory_Motor | Dmkn | 0.72 | 0.06 | 1.99 | 0.44 | 0.35 | 1.94 |
| Excitatory_Motor | Dock2 | 0.77 | 0.14 | 2.11 | 0.49 | 0.14 | 0.59 |
| Excitatory_Motor | Ebf3 | 0.95 | 2.43 | 1.12 | 0.59 | 0.96 | 0.80 |
| Excitatory_Motor | Efna5 | 1.00 | 4.31 | 0.49 | 0.83 | 2.51 | 1.09 |
| Excitatory_Motor | Elavl2 | 0.73 | 0.31 | 1.04 | 0.42 | 0.06 | 0.91 |
| Excitatory_Motor | Epb4.1l4b | 0.59 | −0.80 | 0.17 | 0.65 | 1.09 | 0.72 |
| Excitatory_Motor | Epha4 | 0.55 | 0.22 | 0.73 | 0.29 | −0.68 | 0.75 |
| Excitatory_Motor | Epha7 | 0.57 | 0.34 | 1.65 | 0.60 | 0.94 | 0.96 |
| Excitatory_Motor | Fam163a | 0.76 | 0.24 | 0.22 | 0.68 | 1.43 | 0.57 |
| Excitatory_Motor | Fam19a5 | 0.98 | 3.53 | 1.73 | 0.56 | 0.66 | 1.00 |
| Excitatory_Motor | Fbxo44 | 0.46 | −1.46 | 0.41 | 0.48 | 0.21 | 0.64 |
| Excitatory_Motor | Frmd4b | 0.99 | 3.70 | 0.90 | 0.82 | 2.05 | 1.19 |
| Excitatory_Motor | Gda | 0.49 | 0.67 | 3.17 | 0.51 | 0.35 | 0.82 |
| Excitatory_Motor | Gfra2 | 0.80 | 1.47 | 1.51 | 0.38 | −0.12 | 0.69 |
| Excitatory_Motor | Gpc6 | 1.00 | 5.11 | 1.71 | 0.99 | 4.73 | 2.45 |
| Excitatory_Motor | Gpr22 | 0.52 | −1.39 | 0.38 | 0.39 | −0.04 | 1.34 |
| Excitatory_Motor | Gria1 | 0.95 | 2.88 | 1.07 | 0.46 | 0.36 | 1.35 |
| Excitatory_Motor | Gria2 | 0.98 | 1.30 | 0.29 | 0.49 | 0.27 | 0.50 |
| Excitatory_Motor | Grip1 | 1.00 | 4.01 | 1.40 | 0.76 | 1.68 | 1.21 |
| Excitatory_Motor | Hddc2 | 0.39 | −1.73 | 1.15 | 0.43 | 0.36 | 0.61 |
| Excitatory_Motor | Htr4 | 0.87 | 0.76 | 1.78 | 0.67 | 1.34 | 2.31 |
| Excitatory_Motor | Kcnq2 | 0.97 | 2.23 | 0.80 | 0.22 | −1.32 | 1.69 |
| Excitatory_Motor | Kcns3 | 0.72 | 0.15 | 2.36 | 0.54 | 0.68 | 1.43 |
| Excitatory_Motor | Klhl29 | 0.99 | 3.41 | 0.57 | 0.70 | 1.38 | 0.95 |
| Excitatory_Motor | Lbh | 0.36 | −2.40 | 0.14 | 0.38 | 0.39 | 0.93 |
| Excitatory_Motor | Lgi1 | 0.80 | 0.76 | 0.91 | 0.35 | −0.26 | 0.71 |
| Excitatory_Motor | Lrfn2 | 0.75 | 1.04 | 0.52 | 0.53 | 0.41 | 2.23 |
| Excitatory_Motor | Lrig3 | 0.49 | −1.39 | 2.76 | 0.22 | −1.02 | 0.93 |
| Excitatory_Motor | Nfib | 0.94 | 2.26 | 1.12 | 0.62 | 1.06 | 0.52 |
| Excitatory_Motor | Ogdhl | 0.48 | −1.50 | 0.47 | 0.32 | −0.30 | 0.70 |
| Excitatory_Motor | Oprk1 | 0.90 | 2.82 | 4.80 | 0.21 | −1.28 | 1.69 |
| Excitatory_Motor | Pknox2 | 0.62 | 0.37 | 1.28 | 0.45 | 0.30 | 1.31 |
| Excitatory_Motor | Plcxd3 | 0.99 | 2.86 | 1.89 | 0.57 | 0.94 | 0.38 |
| Excitatory_Motor | Plxna2 | 0.91 | 1.42 | 1.53 | 0.64 | 1.05 | 0.66 |
| Excitatory_Motor | Prickle2 | 0.98 | 3.21 | 1.22 | 0.71 | 1.37 | 2.00 |
| Excitatory_Motor | Psd3 | 0.88 | 0.81 | 1.63 | 0.90 | 2.61 | 0.67 |
| Excitatory_Motor | Ptn | 0.82 | 0.59 | 1.15 | 0.33 | −0.60 | 0.17 |
| Excitatory_Motor | Ramp1 | 0.80 | 0.66 | 0.12 | 0.71 | 1.92 | 1.09 |
| Excitatory_Motor | Rbfox1 | 1.00 | 7.57 | 1.65 | 1.00 | 5.52 | 1.21 |
| Excitatory_Motor | Rgs4 | 0.51 | 0.48 | 0.65 | 0.27 | −0.69 | 1.12 |
| Excitatory_Motor | Runx1t1 | 0.52 | −0.71 | 2.50 | 0.40 | 0.19 | 0.80 |
| Excitatory_Motor | Sec14l5 | 0.27 | −3.25 | 1.98 | 0.39 | −0.15 | 0.80 |
| Excitatory_Motor | Sgpp2 | 1.00 | 2.78 | 0.87 | 0.58 | 0.67 | 0.47 |
| Excitatory_Motor | Slc5a7 | 0.75 | 0.34 | 1.42 | 0.77 | 1.90 | 2.31 |
| Excitatory_Motor | Sorbs2 | 0.82 | 0.52 | 0.35 | 0.86 | 2.78 | 0.87 |
| Excitatory_Motor | Spata17 | 0.65 | −2.89 | 0.52 | 0.45 | 0.32 | 0.73 |
| Excitatory_Motor | Specc1 | 0.91 | 1.63 | 1.23 | 0.40 | −0.32 | 0.67 |
| Excitatory_Motor | St5 | 0.49 | −1.20 | 0.52 | 0.62 | 0.89 | 0.49 |
| Excitatory_Motor | St6galnac3 | 0.98 | 2.57 | 2.06 | 0.64 | 1.01 | 0.17 |
| Excitatory_Motor | Syndig1 | 0.91 | 1.54 | 0.18 | 0.47 | 0.47 | 1.16 |
| Excitatory_Motor | Syt6 | 0.98 | 3.53 | 1.41 | 0.38 | −0.20 | 1.84 |
| Excitatory_Motor | Tmem132c | 0.82 | 1.75 | 3.09 | 0.77 | 2.88 | 2.63 |
| Excitatory_Motor | Tmem164 | 0.92 | 1.78 | 1.76 | 0.48 | 0.35 | 0.76 |
| Excitatory_Motor | Tox | 0.99 | 3.64 | 1.61 | 0.88 | 2.80 | 0.85 |
| Excitatory_Motor | Tpd52l1 | 0.96 | 1.09 | 2.42 | 0.88 | 2.37 | 1.82 |
| Excitatory_Motor | Ubash3b | 0.82 | 1.03 | 1.38 | 0.48 | 0.27 | 0.60 |
| Excitatory_Motor | Unc5d | 0.96 | 4.26 | 1.97 | 0.97 | 4.57 | 1.98 |
| Excitatory_Motor | Xylt1 | 0.98 | 2.95 | 1.68 | 0.92 | 3.31 | 1.36 |
| Excitatory_Motor | Zfp521 | 0.99 | 3.33 | 1.08 | 0.59 | 1.01 | 0.83 |
| Inhibitory_Motor | Ablim2 | 1.00 | 4.43 | 1.52 | 0.72 | 1.95 | 0.66 |
| Inhibitory_Motor | Adcy2 | 0.98 | 0.91 | 1.11 | 0.40 | −0.14 | 0.45 |
| Inhibitory_Motor | Add3 | 0.98 | 1.70 | 1.35 | 0.74 | 1.93 | 0.93 |
| Inhibitory_Motor | Aff1 | 0.69 | 0.01 | 0.84 | 0.61 | 1.21 | 0.86 |
| Inhibitory_Motor | Alad | 0.44 | −1.35 | 0.86 | 0.38 | 0.05 | 0.62 |
| Inhibitory_Motor | Alcam | 1.00 | 3.30 | 2.35 | 0.85 | 3.41 | 1.26 |
| Inhibitory_Motor | Aldh1a3 | 0.40 | −0.70 | 4.57 | 0.23 | −0.87 | 0.49 |
| Inhibitory_Motor | Ano4 | 0.80 | −1.30 | 1.85 | 0.40 | −0.13 | 0.66 |
| Inhibitory_Motor | Arhgef26 | 0.41 | −1.21 | 1.96 | 0.28 | −0.86 | 0.63 |
| Inhibitory_Motor | Arid5b | 0.99 | 2.33 | 1.22 | 0.45 | 0.26 | 0.46 |
| Inhibitory_Motor | Atp2b1 | 0.98 | 1.87 | 0.77 | 0.46 | 0.41 | 0.67 |
| Inhibitory_Motor | Cartpt | 0.27 | −1.23 | 1.03 | 0.41 | 3.26 | 3.18 |
| Inhibitory_Motor | Ccdc129 | 0.49 | −4.30 | 0.57 | 0.23 | −1.25 | 0.29 |
| Inhibitory_Motor | Chd7 | 0.95 | 2.40 | 0.98 | 0.39 | −0.10 | 1.06 |
| Inhibitory_Motor | Cit | 0.64 | −1.53 | 0.85 | 0.53 | 0.70 | 1.12 |
| Inhibitory_Motor | Clvs1 | 1.00 | 3.83 | 1.19 | 0.57 | 0.76 | 0.38 |
| Inhibitory_Motor | Col5a2 | 0.91 | 0.77 | 4.02 | 0.52 | 0.87 | 0.52 |
| Inhibitory_Motor | Creb3l2 | 0.53 | −1.11 | 0.56 | 0.38 | −0.04 | 0.64 |
| Inhibitory_Motor | Cryab | 0.25 | −2.80 | 1.63 | 0.43 | 0.97 | 0.88 |
| Inhibitory_Motor | Cygb | 0.56 | −0.67 | 2.47 | 0.23 | −0.59 | 1.75 |
| Inhibitory_Motor | Dach1 | 0.76 | 1.46 | 1.41 | 0.58 | 1.36 | 1.30 |
| Inhibitory_Motor | Dcc | 0.91 | 1.70 | 1.15 | 0.70 | 2.21 | 1.39 |
| Inhibitory_Motor | Dgkb | 1.00 | 3.89 | 3.49 | 0.81 | 3.88 | 1.94 |
| Inhibitory_Motor | Entpd3 | 0.99 | 2.30 | 1.64 | 0.70 | 2.10 | 0.78 |
| Inhibitory_Motor | Epb4.1l2 | 0.83 | −0.08 | 0.24 | 0.59 | 1.09 | 1.34 |
| Inhibitory_Motor | Etv1 | 1.00 | 3.30 | 1.57 | 0.61 | 1.47 | 0.91 |
| Inhibitory_Motor | Fam13c | 0.95 | 0.92 | 1.09 | 0.33 | −0.54 | 0.82 |
| Inhibitory_Motor | Fam78b | 1.00 | 2.94 | 0.72 | 0.58 | 1.12 | 1.13 |
| Inhibitory_Motor | Fgd4 | 0.99 | 0.75 | 0.62 | 0.72 | 1.79 | 0.71 |
| Inhibitory_Motor | Fosl2 | 0.57 | −0.77 | 1.10 | 0.24 | −0.88 | 1.13 |
| Inhibitory_Motor | Fsip1 | 0.36 | −3.59 | 0.47 | 0.34 | −0.36 | 0.83 |
| Inhibitory_Motor | Gal | 0.54 | 0.86 | 0.13 | 0.65 | 4.03 | 1.59 |
| Inhibitory_Motor | Gfra1 | 0.99 | 3.68 | 2.93 | 0.58 | 1.18 | 0.60 |
| Inhibitory_Motor | Gpr176 | 0.78 | 0.24 | 1.30 | 0.59 | 1.13 | 0.81 |
| Inhibitory_Motor | Kcnc1 | 0.85 | 0.75 | 1.43 | 0.23 | −0.74 | 2.71 |
| Inhibitory_Motor | Kcng3 | 0.94 | 1.17 | 0.65 | 0.43 | 0.09 | 0.89 |
| Inhibitory_Motor | Kcnj5 | 0.85 | 1.25 | 2.86 | 0.31 | −0.45 | 1.99 |
| Inhibitory_Motor | Kcnq4 | 0.93 | 0.81 | 2.13 | 0.21 | −1.36 | 0.76 |
| Inhibitory_Motor | Kcnt2 | 1.00 | 4.08 | 1.37 | 0.75 | 2.35 | 0.11 |
| Inhibitory_Motor | Kirrel3 | 0.90 | 3.72 | 0.96 | 0.44 | 0.20 | 0.50 |
| Inhibitory_Motor | Klf7 | 0.97 | 2.18 | 0.60 | 0.48 | 0.37 | 0.26 |
| Inhibitory_Motor | Lama5 | 0.69 | 0.57 | 1.67 | 0.25 | −1.05 | 0.69 |
| Inhibitory_Motor | Lima1 | 0.80 | −0.87 | 0.21 | 0.69 | 1.52 | 0.52 |
| Inhibitory_Motor | Lrch1 | 0.95 | 1.52 | 0.93 | 0.45 | 0.30 | 0.55 |
| Inhibitory_Motor | Lrig2 | 0.98 | 2.24 | 0.61 | 0.44 | 0.19 | 0.77 |
| Inhibitory_Motor | Lrriq1 | 0.51 | −2.77 | 0.04 | 0.43 | 0.26 | 0.98 |
| Inhibitory_Motor | Man1a | 0.97 | 2.90 | 1.97 | 0.69 | 2.28 | 0.82 |
| Inhibitory_Motor | Man2a1 | 0.99 | 3.16 | 0.54 | 0.48 | 0.52 | 0.77 |
| Inhibitory_Motor | Mkx | 0.44 | −1.14 | 2.40 | 0.28 | −0.76 | 0.83 |
| Inhibitory_Motor | Ncald | 1.00 | 2.94 | 1.49 | 0.79 | 2.27 | 0.72 |
| Inhibitory_Motor | Net1 | 0.23 | −3.20 | 0.44 | 0.22 | −1.17 | 1.13 |
| Inhibitory_Motor | Nos1 | 1.00 | 4.77 | 5.29 | 0.87 | 4.44 | 3.19 |
| Inhibitory_Motor | Oprd1 | 0.90 | 1.42 | 2.58 | 0.43 | 0.27 | 1.53 |
| Inhibitory_Motor | Pald1 | 0.70 | −0.47 | 1.35 | 0.26 | −0.98 | 1.24 |
| Inhibitory_Motor | Pde1a | 0.97 | 1.64 | 2.41 | 0.66 | 2.31 | 2.09 |
| Inhibitory_Motor | Pde1c | 0.99 | 3.41 | 1.52 | 0.61 | 1.31 | 0.42 |
| Inhibitory_Motor | Pik3c2g | 0.80 | −2.55 | 0.88 | 0.51 | 0.70 | 0.51 |
| Inhibitory_Motor | Prkg2 | 0.36 | −1.76 | 0.35 | 0.33 | −0.53 | 0.26 |
| Inhibitory_Motor | Ptgir | 0.23 | −1.39 | 2.23 | 0.31 | −0.25 | 1.40 |
| Inhibitory_Motor | Ptpn13 | 0.53 | −1.32 | 0.83 | 0.44 | 0.14 | 0.51 |
| Inhibitory_Motor | Ptprg | 1.00 | 5.00 | 1.09 | 0.86 | 3.36 | 1.10 |
| Inhibitory_Motor | Pxdn | 0.96 | 1.66 | 0.79 | 0.31 | −0.33 | 2.10 |
| Inhibitory_Motor | Qdpr | 0.76 | 0.42 | 1.60 | 0.54 | 1.32 | 0.84 |
| Inhibitory_Motor | Rnf125 | 0.41 | −3.06 | 0.84 | 0.45 | 0.28 | 0.68 |
| Inhibitory_Motor | Samd5 | 0.69 | −0.04 | 1.10 | 0.27 | −0.23 | 1.54 |
| Inhibitory_Motor | Serinc5 | 0.70 | −0.69 | 0.40 | 0.62 | 1.13 | 0.80 |
| Inhibitory_Motor | Sipa1l2 | 0.87 | 1.13 | 1.48 | 0.28 | −0.83 | 0.93 |
| Inhibitory_Motor | Slc16a1 | 0.42 | −1.49 | 1.47 | 0.26 | −0.86 | 1.05 |
| Inhibitory_Motor | Slc4a4 | 0.90 | 2.08 | 0.56 | 0.50 | 0.62 | 1.26 |
| Inhibitory_Motor | Sntb1 | 1.00 | 3.83 | 1.35 | 0.69 | 1.98 | 1.68 |
| Inhibitory_Motor | Sobp | 0.99 | 2.62 | 0.82 | 0.45 | 0.37 | 0.77 |
| Inhibitory_Motor | St18 | 1.00 | 1.18 | 1.42 | 0.60 | 1.37 | 2.26 |
| Inhibitory_Motor | St3gal4 | 0.42 | −1.72 | 0.46 | 0.30 | −0.55 | 0.65 |
| Inhibitory_Motor | Stac2 | 0.70 | 0.49 | 0.54 | 0.23 | −0.67 | 1.10 |
| Inhibitory_Motor | Stard13 | 0.99 | 2.33 | 1.70 | 0.52 | 0.72 | 0.96 |
| Inhibitory_Motor | Tanc1 | 0.65 | −0.53 | 1.82 | 0.66 | 1.70 | 1.63 |
| Inhibitory_Motor | Tbx3 | 0.83 | 0.57 | 0.78 | 0.27 | −0.50 | 0.62 |
| Inhibitory_Motor | Tctex1d1 | 0.51 | −2.27 | 0.93 | 0.32 | −0.26 | 3.13 |
| Inhibitory_Motor | Tenm3 | 0.97 | 2.99 | 1.62 | 0.74 | 2.11 | 0.40 |
| Inhibitory_Motor | Tmco4 | 0.38 | −2.31 | 0.80 | 0.38 | −0.22 | 0.64 |
| Inhibitory_Motor | Tnfrsf25 | 0.38 | −0.92 | 1.26 | 0.25 | −0.61 | 1.41 |
| Inhibitory_Motor | Tnr | 0.86 | 1.78 | 0.38 | 0.26 | −0.79 | 0.86 |
| Inhibitory_Motor | Tpst1 | 0.56 | −1.06 | 0.51 | 0.82 | 3.31 | 1.33 |
| Inhibitory_Motor | Utrn | 1.00 | 3.19 | 0.57 | 0.60 | 1.17 | 0.81 |
| Inhibitory_Motor | Vip | 0.41 | 1.84 | 0.42 | 0.56 | 4.17 | 0.67 |
| Inhibitory_Motor | Wipi1 | 0.91 | 1.05 | 1.97 | 0.24 | −1.03 | 0.89 |
| Inhibitory_Motor | Zeb2 | 0.97 | 2.29 | 0.81 | 0.56 | 1.00 | 0.90 |
| Inhibitory_Motor | Zfp536 | 0.98 | 3.10 | 1.73 | 0.64 | 1.51 | 0.64 |
| Inhibitory_Motor | Zfyve16 | 0.78 | −1.01 | 0.12 | 0.52 | 0.60 | 0.46 |
| Interneuron | Abcc8 | 1.00 | 2.75 | 1.87 | 0.73 | 0.91 | 1.27 |
| Interneuron | Adra2a | 0.57 | −1.29 | 2.45 | 0.30 | −1.25 | 1.65 |
| Interneuron | B3gnt2 | 0.84 | 0.79 | 0.25 | 0.31 | −1.57 | 1.14 |
| Interneuron | Chgb | 0.62 | 0.43 | 0.48 | 0.65 | 1.78 | 1.62 |
| Interneuron | Clstn2 | 0.89 | 1.47 | 0.87 | 0.80 | 1.61 | 1.98 |
| Interneuron | Cntn3 | 0.90 | 1.08 | 0.86 | 0.73 | 1.07 | 2.00 |
| Interneuron | Ctxn1 | 0.67 | −0.59 | 1.29 | 0.28 | −1.51 | 1.26 |
| Interneuron | Dapk1 | 0.99 | 1.54 | 2.35 | 0.69 | 0.45 | 0.87 |
| Interneuron | Dynlt3 | 0.61 | 0.36 | 1.42 | 0.61 | 0.67 | 0.87 |
| Interneuron | Eef1a2 | 0.54 | −2.19 | 0.46 | 0.45 | −0.02 | 0.98 |
| Interneuron | Elovl4 | 0.66 | 0.38 | 1.62 | 0.47 | −0.59 | 1.14 |
| Interneuron | Emb | 0.85 | 0.64 | 0.99 | 0.42 | −0.82 | 1.91 |
| Interneuron | Fam196b | 0.63 | 0.29 | 1.54 | 0.27 | −1.92 | 1.96 |
| Interneuron | Fam19a2 | 0.99 | 3.36 | 1.65 | 0.86 | 2.75 | 1.65 |
| Interneuron | Fam219b | 0.56 | −1.00 | 0.43 | 0.70 | 0.26 | 0.57 |
| Interneuron | Gabarapl1 | 0.89 | 1.68 | 1.06 | 0.76 | 1.76 | 1.10 |
| Interneuron | Hpcal4 | 0.58 | −0.32 | 1.01 | 0.23 | −1.18 | 1.64 |
| Interneuron | Igf2bp2 | 0.54 | −1.30 | 2.28 | 0.73 | 0.59 | 0.91 |
| Interneuron | Irf2bpl | 0.47 | −2.15 | 0.72 | 0.31 | −0.95 | 1.47 |
| Interneuron | Kcnc4 | 0.68 | −0.40 | 1.04 | 0.50 | −0.02 | 1.48 |
| Interneuron | Lbh | 0.80 | −0.52 | 2.44 | 0.49 | 1.08 | 1.60 |
| Interneuron | Lin7a | 0.91 | 0.99 | 1.11 | 0.62 | 0.96 | 2.74 |
| Interneuron | Lnpep | 0.99 | 2.01 | 1.08 | 0.77 | 0.88 | 0.40 |
| Interneuron | Meis1 | 0.99 | 2.39 | 1.82 | 0.82 | 1.82 | 1.30 |
| Interneuron | Mt3 | 0.57 | −2.69 | 2.59 | 0.53 | 0.54 | 1.41 |
| Interneuron | Ndufaf3 | 0.41 | −2.25 | 0.75 | 0.45 | −0.56 | 0.64 |
| Interneuron | Nefm | 0.44 | 0.99 | 2.54 | 0.55 | 1.34 | 2.33 |
| Interneuron | Nfatc1 | 0.65 | 1.94 | 7.15 | 0.41 | −0.33 | 2.06 |
| Interneuron | Nrp2 | 0.99 | 3.01 | 1.21 | 0.73 | 1.56 | 1.90 |
| Interneuron | Ogfrl1 | 0.47 | −1.41 | 1.27 | 0.57 | −0.14 | 0.97 |
| Interneuron | Parva | 1.00 | 3.39 | 1.97 | 0.78 | 1.18 | 0.84 |
| Interneuron | Penk | 0.86 | 5.03 | 5.99 | 0.70 | 4.85 | 4.89 |
| Interneuron | Phox2a | 0.51 | −3.10 | 1.41 | 0.51 | 0.23 | 1.33 |
| Interneuron | Prickle2 | 0.99 | 2.90 | 0.53 | 0.70 | 0.70 | 0.68 |
| Interneuron | Ptprz1 | 0.73 | 1.68 | 1.79 | 0.68 | 0.72 | 1.52 |
| Interneuron | Rgs4 | 0.55 | 1.92 | 2.54 | 0.32 | 0.31 | 2.19 |
| Interneuron | Sdc3 | 0.77 | 0.23 | 0.25 | 0.38 | −0.80 | 1.30 |
| Interneuron | Sema3e | 0.69 | 3.39 | 4.88 | 0.84 | 2.01 | 1.74 |
| Interneuron | Slc16a12 | 0.68 | −0.02 | 1.67 | 0.51 | 0.15 | 1.90 |
| Interneuron | Slc1a4 | 0.73 | 0.39 | 1.10 | 0.65 | 0.42 | 1.36 |
| Interneuron | Sncg | 0.87 | 1.13 | 1.23 | 0.91 | 3.54 | 0.96 |
| Interneuron | Spock1 | 0.88 | 2.54 | 1.26 | 0.78 | 1.56 | 1.30 |
| Interneuron | Tac1 | 0.64 | 3.72 | 3.62 | 0.42 | 1.80 | 0.40 |
| Interneuron | Tbx2 | 0.22 | −1.97 | 1.83 | 0.27 | −1.30 | 1.44 |
| Interneuron | Tenm2 | 0.96 | 4.27 | 0.71 | 0.96 | 3.83 | 2.06 |
| Interneuron | Tlx2 | 0.42 | −1.58 | 0.73 | 0.53 | 0.43 | 0.95 |
| Interneuron | Tm4sf4 | 0.93 | 2.16 | 1.60 | 0.70 | 1.97 | 2.22 |
| Interneuron | Tmc3 | 0.73 | 2.14 | 1.56 | 0.69 | 1.30 | 2.11 |
| Interneuron | Tns3 | 1.00 | 3.98 | 0.58 | 0.86 | 1.63 | 0.73 |
| Interneuron | Tomm22 | 0.47 | −1.92 | 0.81 | 0.42 | −0.34 | 0.99 |
| Interneuron | Trim36 | 0.70 | −0.69 | 1.76 | 0.54 | −0.19 | 1.01 |
| Interneuron | Tspyl1 | 0.63 | 0.51 | 0.95 | 0.61 | 1.76 | 1.02 |
| Interneuron | Ush1c | 0.94 | 1.49 | 2.51 | 0.51 | 0.62 | 3.75 |
| Interneuron | Vstm2a | 0.27 | −1.87 | 2.13 | 0.41 | 0.44 | 2.33 |
| Interneuron | Ypel5 | 0.78 | 0.61 | 1.33 | 0.41 | −0.85 | 0.66 |
| Interneuron | Zfhx3 | 0.97 | 2.22 | 2.50 | 0.84 | 1.48 | 0.81 |
| Interneuron | Zmat4 | 0.88 | 0.78 | 3.07 | 0.80 | 1.84 | 1.73 |
| Secretomotor | Abca5 | 0.88 | 0.11 | 0.05 | 0.67 | 1.72 | 1.03 |
| Secretomotor | Adamts1 | 0.31 | −0.38 | 1.67 | 0.36 | 0.42 | 1.12 |
| Secretomotor | Arnt2 | 0.74 | −0.69 | 0.85 | 0.53 | 1.14 | 1.64 |
| Secretomotor | Arpp21 | 1.00 | 3.72 | 2.02 | 0.57 | 1.53 | 1.50 |
| Secretomotor | Calb2 | 0.97 | 0.83 | 1.70 | 0.85 | 3.58 | 3.94 |
| Secretomotor | Camk2a | 1.00 | 3.28 | 1.59 | 0.35 | 0.82 | 1.50 |
| Secretomotor | Camk4 | 1.00 | 3.10 | 1.47 | 0.93 | 3.37 | 1.34 |
| Secretomotor | Cdh10 | 0.86 | 0.50 | 3.15 | 0.68 | 1.77 | 1.60 |
| Secretomotor | Chdh | 0.44 | −1.29 | 0.78 | 0.33 | 0.08 | 2.18 |
| Secretomotor | Clic5 | 0.30 | −1.66 | 3.75 | 0.46 | 0.41 | 0.98 |
| Secretomotor | Cntn3 | 0.94 | 1.09 | 0.91 | 0.38 | 0.31 | 1.03 |
| Secretomotor | Cux2 | 0.89 | 1.52 | 1.90 | 0.67 | 1.86 | 0.99 |
| Secretomotor | Dennd5a | 0.84 | −0.03 | 0.34 | 0.60 | 1.23 | 0.75 |
| Secretomotor | Ell2 | 0.77 | 0.41 | 1.16 | 0.65 | 1.87 | 1.81 |
| Secretomotor | Etv1 | 1.00 | 4.13 | 1.78 | 0.81 | 2.51 | 1.62 |
| Secretomotor | Fmn1 | 0.97 | 1.37 | 1.78 | 0.58 | 1.90 | 1.83 |
| Secretomotor | Gal | 0.58 | 3.72 | 3.96 | 0.64 | 4.64 | 1.28 |
| Secretomotor | Gan | 0.46 | −1.19 | 0.78 | 0.67 | 1.79 | 1.60 |
| Secretomotor | Gfra1 | 0.98 | 3.66 | 1.11 | 0.71 | 2.27 | 1.55 |
| Secretomotor | Gng8 | 0.25 | −3.20 | 2.55 | 0.29 | −0.22 | 0.78 |
| Secretomotor | Hcn1 | 0.93 | 0.64 | 0.98 | 0.63 | 1.65 | 1.14 |
| Secretomotor | Kcnc2 | 1.00 | 3.90 | 0.48 | 0.43 | 0.67 | 1.21 |
| Secretomotor | Kcnd2 | 1.00 | 6.04 | 2.65 | 0.94 | 4.09 | 2.01 |
| Secretomotor | Kcnk2 | 0.89 | 0.39 | 0.79 | 0.69 | 1.79 | 2.95 |
| Secretomotor | Kif26a | 0.77 | −0.06 | 1.03 | 0.29 | −0.20 | 1.65 |
| Secretomotor | Lhfpl2 | 0.42 | −1.26 | 0.93 | 0.60 | 1.36 | 1.14 |
| Secretomotor | Luzp2 | 0.98 | 1.49 | 2.79 | 0.81 | 2.48 | 1.42 |
| Secretomotor | Nol4 | 0.98 | 1.44 | 0.71 | 0.61 | 1.47 | 1.46 |
| Secretomotor | Npy2r | 0.28 | −1.14 | 2.33 | 0.25 | −0.30 | 2.30 |
| Secretomotor | Ntsr1 | 0.36 | −1.52 | 2.42 | 0.25 | −0.61 | 1.75 |
| Secretomotor | Pcdh17 | 0.47 | −0.92 | 1.94 | 0.44 | 0.75 | 0.90 |
| Secretomotor | Plcz1 | 0.38 | −2.12 | 1.24 | 0.24 | −0.98 | 1.27 |
| Secretomotor | Plscr4 | 0.87 | 0.60 | 0.75 | 0.28 | −0.66 | 1.07 |
| Secretomotor | Plxna4 | 0.80 | 3.82 | 1.52 | 0.61 | 1.67 | 1.77 |
| Secretomotor | Prkd1 | 0.96 | 1.72 | 2.79 | 0.54 | 0.89 | 0.50 |
| Secretomotor | Prkg2 | 0.55 | −0.10 | 2.16 | 0.49 | 1.09 | 2.07 |
| Secretomotor | Robo2 | 0.97 | 1.73 | 0.61 | 0.88 | 3.52 | 0.56 |
| Secretomotor | Scgn | 0.94 | 2.84 | 4.40 | 0.90 | 3.22 | 2.46 |
| Secretomotor | Scn11a | 0.92 | 0.66 | 1.27 | 0.47 | 0.97 | 0.93 |
| Secretomotor | Slc16a7 | 0.78 | −0.75 | 0.68 | 0.35 | 0.31 | 1.64 |
| Secretomotor | Spock3 | 0.97 | 2.52 | 1.15 | 0.72 | 2.07 | 0.76 |
| Secretomotor | Syt10 | 0.56 | −0.90 | 2.56 | 0.42 | 1.23 | 2.73 |
| Secretomotor | Trps1 | 0.89 | 2.71 | 1.63 | 0.50 | 1.35 | 1.59 |
| Secretomotor | Unc5b | 0.50 | −0.97 | 1.74 | 0.33 | 0.06 | 3.20 |
| Secretomotor | Vip | 0.65 | 4.34 | 3.50 | 0.88 | 5.65 | 2.10 |
| Secretomotor | Zyx | 0.45 | −2.29 | 0.56 | 0.28 | −0.31 | 1.32 |
| Sensory | Ache | 0.92 | 2.25 | 1.15 | 0.67 | 1.41 | 1.15 |
| Sensory | Acp1 | 0.88 | −0.94 | 0.49 | 0.44 | 0.11 | 1.86 |
| Sensory | Adamts1 | 0.34 | −0.24 | 1.88 | 0.56 | 1.27 | 2.04 |
| Sensory | Adap1 | 0.86 | 0.38 | 0.53 | 0.23 | −2.41 | 1.17 |
| Sensory | Adora1 | 0.28 | −0.98 | 2.06 | 0.30 | −1.15 | 2.22 |
| Sensory | Agpat2 | 0.27 | −2.61 | 0.96 | 0.28 | −1.57 | 1.74 |
| Sensory | Ano2 | 0.89 | 2.02 | 4.98 | 0.67 | 1.37 | 1.85 |
| Sensory | Anxa2 | 0.81 | 0.33 | 1.80 | 0.84 | 1.95 | 0.95 |
| Sensory | Arf5 | 0.54 | −3.06 | 0.48 | 0.47 | −0.17 | 1.59 |
| Sensory | Arhgdig | 0.46 | −1.68 | 0.91 | 0.63 | 1.38 | 1.94 |
| Sensory | Atp1a3 | 0.64 | −0.29 | 0.74 | 0.67 | 0.88 | 0.66 |
| Sensory | B2m | 0.59 | −2.26 | 0.79 | 0.93 | 5.12 | 1.84 |
| Sensory | B3galt6 | 0.21 | −2.63 | 0.75 | 0.40 | −0.16 | 3.28 |
| Sensory | Boc | 0.53 | −0.65 | 1.35 | 0.58 | −0.61 | 1.98 |
| Sensory | Calb2 | 0.92 | 0.61 | 1.45 | 0.51 | 1.99 | 1.29 |
| Sensory | Caleb | 0.71 | 2.64 | 5.83 | 0.53 | 1.18 | 1.55 |
| Sensory | Cbln2 | 0.36 | 0.81 | 4.77 | 0.77 | 2.96 | 5.94 |
| Sensory | Cd151 | 0.59 | −0.60 | 0.73 | 0.74 | 0.96 | 0.80 |
| Sensory | Cdc42ep4 | 0.26 | −2.58 | 0.76 | 0.44 | −0.56 | 0.98 |
| Sensory | Cnp | 0.39 | −0.64 | 1.02 | 0.35 | −0.72 | 1.73 |
| Sensory | Cnr1 | 0.99 | 3.37 | 1.26 | 0.81 | 1.75 | 1.03 |
| Sensory | Cntfr | 0.69 | −1.59 | 0.66 | 0.28 | −0.34 | 3.45 |
| Sensory | Cpne5 | 0.43 | −2.08 | 2.92 | 0.65 | 0.79 | 1.48 |
| Sensory | Cxxc4 | 0.61 | −1.09 | 0.23 | 0.58 | 0.53 | 1.42 |
| Sensory | Dcx | 0.61 | −0.14 | 0.96 | 0.40 | −1.16 | 1.03 |
| Sensory | Dgkh | 0.88 | 1.19 | 0.85 | 0.88 | 5.29 | 5.19 |
| Sensory | Dlx3 | 0.21 | −2.69 | 5.93 | 0.37 | −1.50 | 7.05 |
| Sensory | Dpf1 | 0.49 | −3.10 | 0.53 | 0.51 | −0.22 | 1.29 |
| Sensory | Galr1 | 0.29 | 0.32 | 2.41 | 0.40 | −0.28 | 2.78 |
| Sensory | Gap43 | 0.96 | 1.72 | 1.11 | 0.91 | 3.27 | 1.33 |
| Sensory | Gse1 | 0.98 | 1.62 | 1.06 | 0.53 | −0.07 | 1.23 |
| Sensory | Hpca | 0.23 | −1.10 | 4.39 | 0.26 | −1.84 | 4.53 |
| Sensory | Hpcal1 | 0.84 | 0.44 | 1.47 | 0.63 | 0.85 | 1.12 |
| Sensory | Htr3a | 0.45 | 2.40 | 1.21 | 0.84 | 3.02 | 4.02 |
| Sensory | Lgals3bp | 0.54 | 0.00 | 1.59 | 0.49 | −0.13 | 0.64 |
| Sensory | Lin7b | 0.39 | −2.06 | 0.46 | 0.51 | −0.08 | 1.46 |
| Sensory | Maz | 0.77 | −0.74 | 1.19 | 0.40 | −0.93 | 1.00 |
| Sensory | Mmadhc | 0.48 | −1.93 | 0.69 | 0.37 | −0.77 | 1.44 |
| Sensory | Nmu | 0.26 | 0.97 | 7.11 | 0.40 | 0.77 | 4.44 |
| Sensory | Nt5dc2 | 0.22 | −3.12 | 1.07 | 0.49 | −0.12 | 1.03 |
| Sensory | Ntrk3 | 0.91 | 3.95 | 1.09 | 0.74 | 2.68 | 3.51 |
| Sensory | Phox2b | 0.88 | 0.99 | 1.10 | 0.65 | 1.85 | 2.57 |
| Sensory | Pianp | 0.56 | −2.08 | 0.29 | 0.47 | −0.02 | 2.32 |
| Sensory | Plcb3 | 0.70 | 0.12 | 0.74 | 0.44 | 0.25 | 2.30 |
| Sensory | Plscr4 | 0.80 | 0.72 | 0.93 | 0.51 | 0.26 | 2.09 |
| Sensory | Plxna4 | 1.00 | 4.62 | 2.72 | 0.74 | 2.08 | 2.14 |
| Sensory | Pomp | 0.71 | −1.78 | 1.09 | 0.65 | 1.30 | 1.67 |
| Sensory | Psmb8 | 0.28 | −2.83 | 1.44 | 0.33 | −1.68 | 1.20 |
| Sensory | Ptpru | 0.27 | −2.42 | 1.57 | 0.30 | −1.13 | 2.38 |
| Sensory | Rab3b | 0.56 | −0.65 | 0.39 | 0.95 | 3.49 | 1.74 |
| Sensory | Rac3 | 0.46 | −1.88 | 1.08 | 0.53 | 0.51 | 1.60 |
| Sensory | Rom1 | 0.25 | −1.93 | 1.13 | 0.26 | −1.86 | 1.82 |
| Sensory | Rph3a | 0.61 | 0.12 | 5.25 | 0.65 | 0.83 | 1.48 |
| Sensory | Samd11 | 0.60 | −1.48 | 0.41 | 0.58 | 0.60 | 2.50 |
| Sensory | Scn11a | 0.93 | 1.19 | 2.32 | 0.77 | 1.95 | 2.01 |
| Sensory | Scube1 | 1.00 | 3.83 | 2.79 | 0.67 | 1.62 | 3.11 |
| Sensory | Sdc3 | 0.76 | 0.51 | 0.58 | 0.58 | 0.80 | 3.25 |
| Sensory | Slc12a7 | 0.58 | −0.25 | 3.38 | 0.47 | 0.00 | 4.73 |
| Sensory | Slc36a1 | 0.82 | 0.85 | 2.10 | 0.51 | −0.34 | 1.33 |
| Sensory | Smad9 | 0.60 | −1.12 | 1.52 | 0.65 | 0.95 | 1.55 |
| Sensory | Spock2 | 0.98 | 1.70 | 0.99 | 0.74 | 2.72 | 1.75 |
| Sensory | Ssbp4 | 0.55 | −2.08 | 0.40 | 0.56 | 0.16 | 0.93 |
| Sensory | Sst | 0.68 | 2.68 | 6.75 | 0.63 | 2.26 | 6.39 |
| Sensory | Susd2 | 0.44 | 0.53 | 2.72 | 0.42 | −0.53 | 3.50 |
| Sensory | Syt7 | 0.88 | 1.02 | 1.27 | 0.60 | 0.54 | 1.51 |
| Sensory | Tapbp | 0.69 | −0.07 | 0.64 | 0.67 | 0.96 | 1.37 |
| Sensory | Tbx2 | 0.32 | −1.28 | 2.70 | 0.49 | −0.11 | 2.81 |
| Sensory | Tcf7l2 | 0.96 | 2.87 | 2.90 | 0.95 | 2.90 | 2.02 |
| Sensory | Thra | 0.83 | −0.50 | 0.46 | 0.67 | 1.33 | 1.78 |
| Sensory | Tlx2 | 0.61 | −0.22 | 2.79 | 0.63 | 1.18 | 1.75 |
| Sensory | Trp53i11 | 0.71 | 1.18 | 1.72 | 0.77 | 1.80 | 2.78 |
| Sensory | Tspan7 | 0.94 | 1.22 | 0.97 | 0.47 | −0.11 | 0.57 |
| Sensory | Unc5b | 0.52 | −1.58 | 1.27 | 0.35 | −1.02 | 1.46 |
| Sensory | Vgll3 | 0.29 | −0.65 | 2.74 | 0.70 | 1.04 | 3.92 |
| Sensory | Vipr2 | 0.70 | 1.89 | 4.59 | 0.58 | 0.32 | 2.30 |
| Sensory | Zfp804a | 0.69 | 3.05 | 2.27 | 0.88 | 3.84 | 2.69 |

### Example 21

### Improved Single Nuclei RNA-seq Analysis

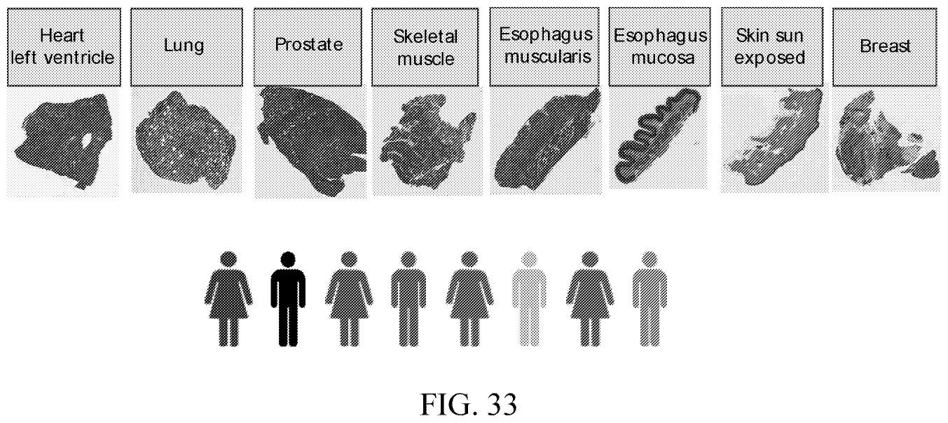

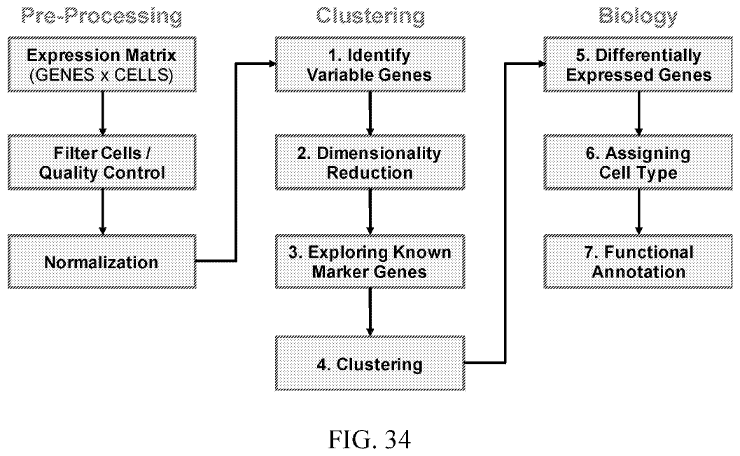

Applicants provide an improved pipeline to apply single-cell genomics to multiple tissue types and multiple individuals (FIG. 33). Applicants provide for methods to solve problems associated with analyzing single nuclei. Applicants provide examples of how analysis of single nuclei transcriptomic data is different from single cell transcriptomic data. Applicants provide examples showing variation of single cell/single nuclei RNA-seq analysis across preps, individuals, and tissues. Applicants provide methods to scale up the approaches to many individuals. FIG. 34 shows the single nuclei RNA-seq analysis pipeline. The pipeline can result in a tSNE plot showing clustering of individual nuclei. The individual nuclei clustered can be classified based on differential expression of genes in each cluster. The clusters can be assigned to a specific cell type based on known marker genes. New cell subtypes can also be identified.

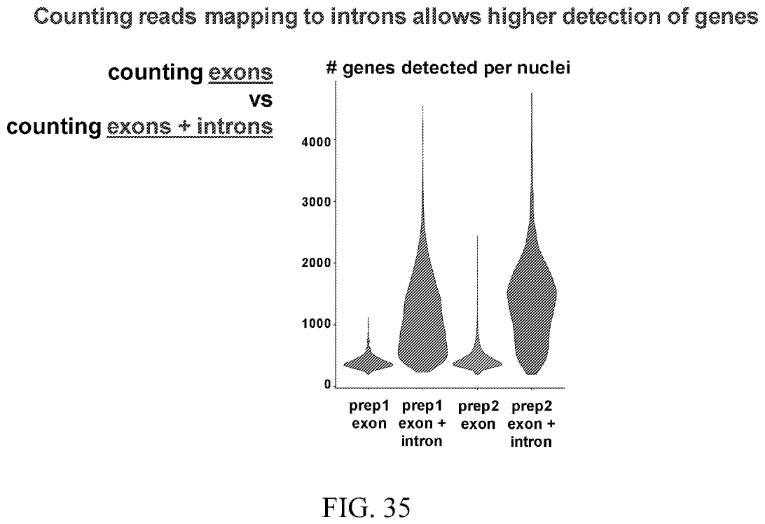

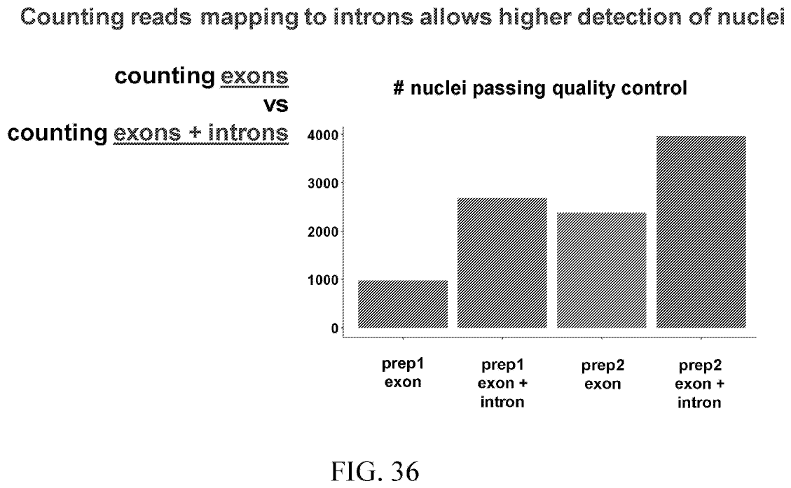

An important difference in single nuclei RNA-seq compared to single cell RNA-seq is that many reads in single nuclei RNA-seq map to introns. Applicants hypothesized that counting reads that map to introns can allow better recovery of biological processes. Applicants determined that counting reads mapping to introns allows higher detection of genes (FIG. 35). Applicants also determined that counting reads mapping to introns allows higher detection of nuclei (FIG. 36).

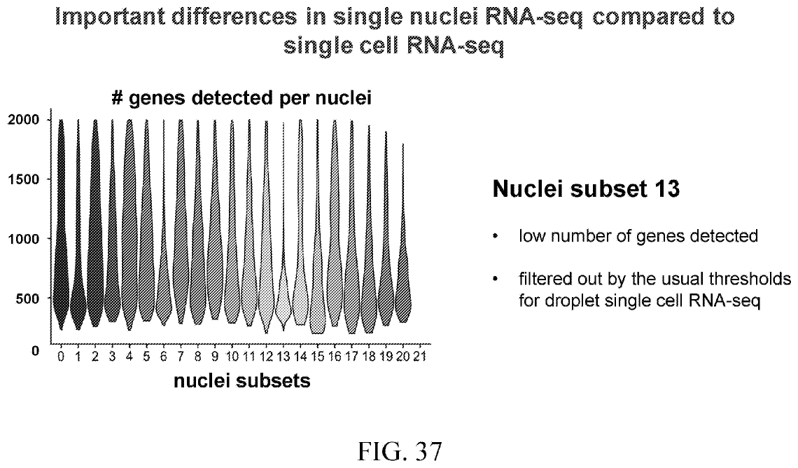

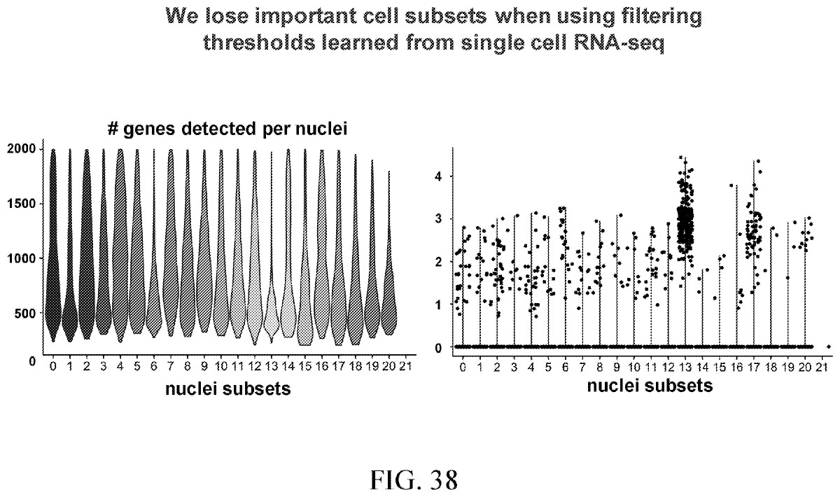

Applicants also determined that filtering using computational methods developed for single cell RNA-seq can lead to low numbers of genes detected and important cell subsets can be lost (FIGS. 37 and 38). For example, T cell receptor is expressed in nuclei subset 13 indicating that this subset are nuclei from T cells. Applicants show that by using thresholds from single cell RNA-seq T cells are lost because the number of genes detected is low.

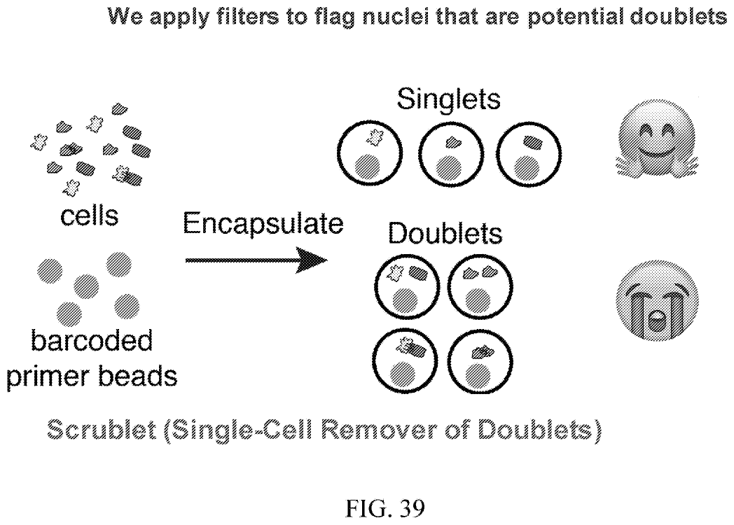

Another issue to overcome is removing nuclei that are potential doublets (FIG. 39). One computational method that can be used to remove doublets is Scrublet (Wolock, Samuel L., Romain Lopez, and Allon M. Klein. “Scrublet: computational identification of cell doublets in single-cell transcriptomic data.” BioRxiv(2018): 3573). Other filtering methods may be used.

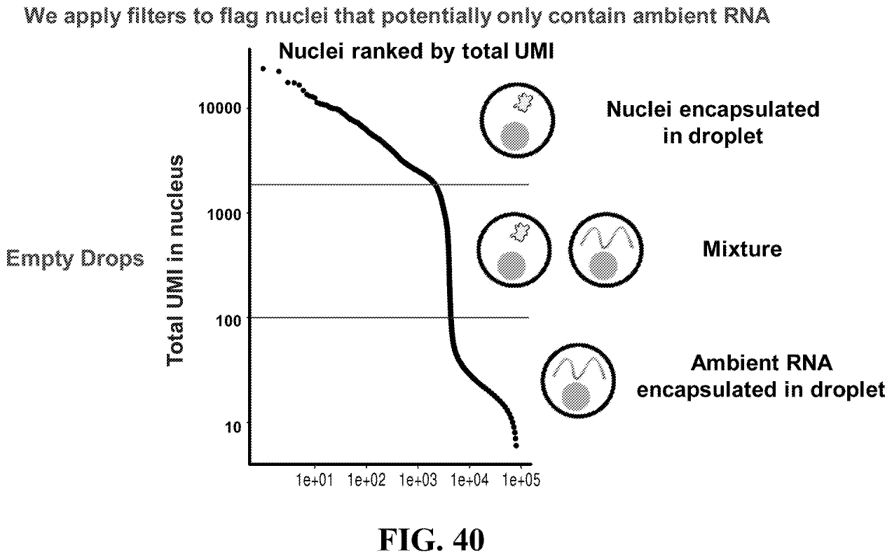

Another issue to overcome is removing nuclei that potentially only contain ambient RNA (FIG. 40). One computational method that can be used for ambient RNA is EmptyDrops (Lun, Aaron T L, et al. “EmptyDrops: distinguishing cells from empty droplets in droplet-based single-cell RNA sequencing data.” Genome biology 20.1 (2019): 63). The total number of unique molecular identifiers (UMI) can be used to distinguish the nuclei, as nuclei encapsulated in droplets have a higher rank and greater total UMIs.

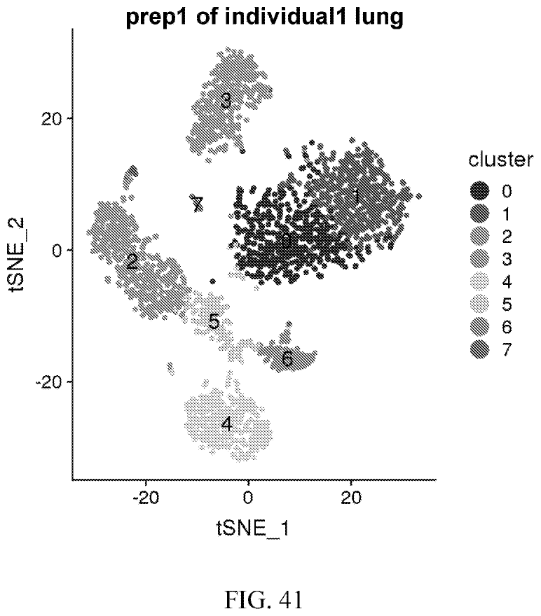

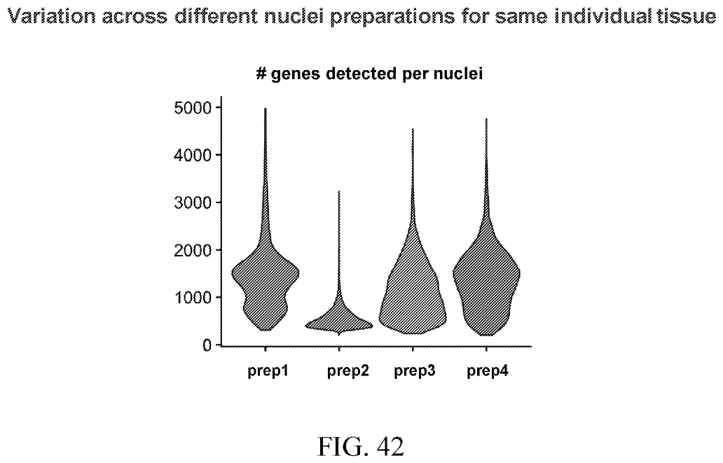

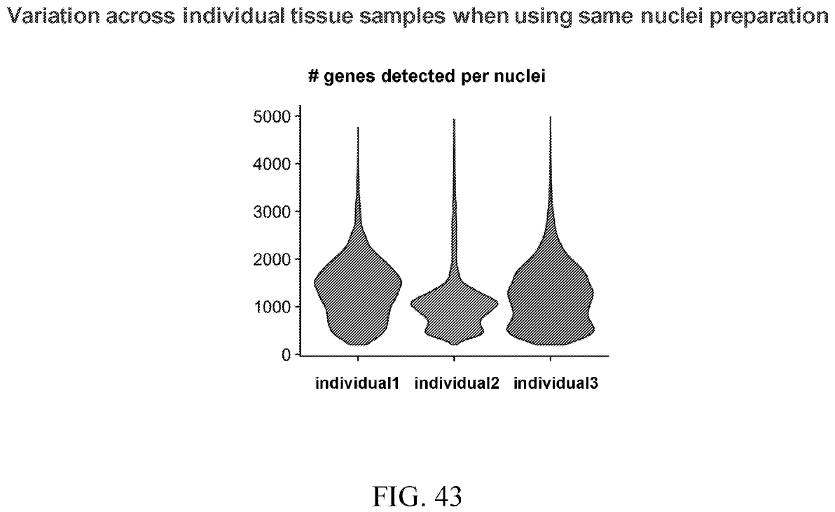

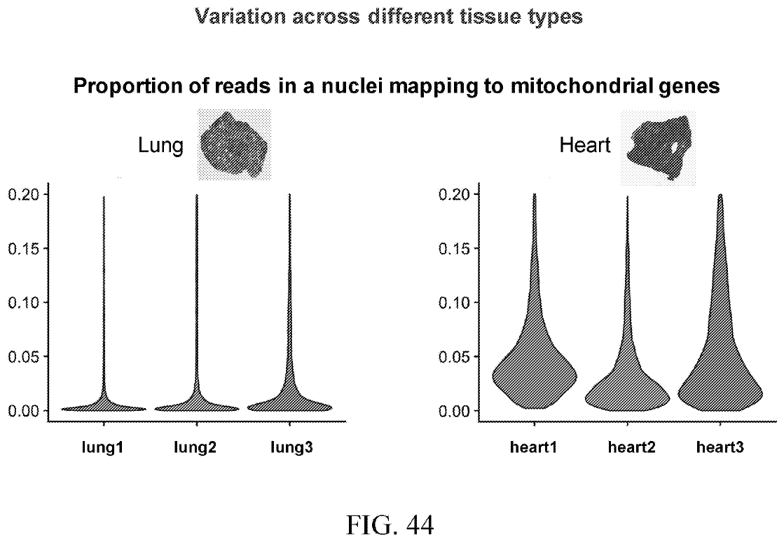

Applicants successfully clustered lung cell subsets using single nuclei RNA-seq (FIG. 41). Applicants observed variation across different nuclei preparations for the same individuals (FIG. 42) and across individual tissue samples when using the same nuclei preparation (FIG. 43). Applicants also show that there is variation between tissue types that needs to be accounted for. For example, the proportion of reads mapping to mitochondrial genes is much higher in heart tissue (FIG. 44).

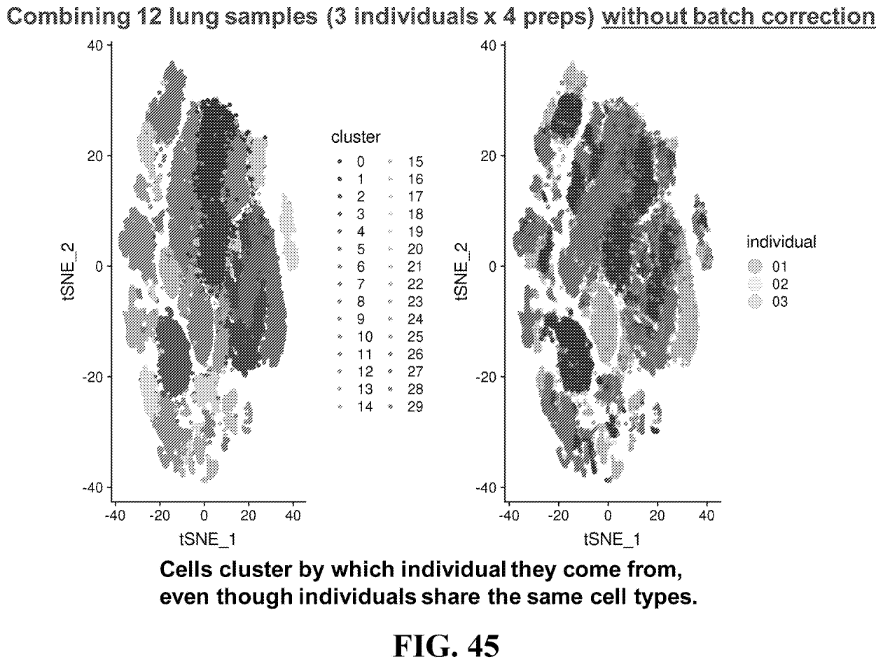

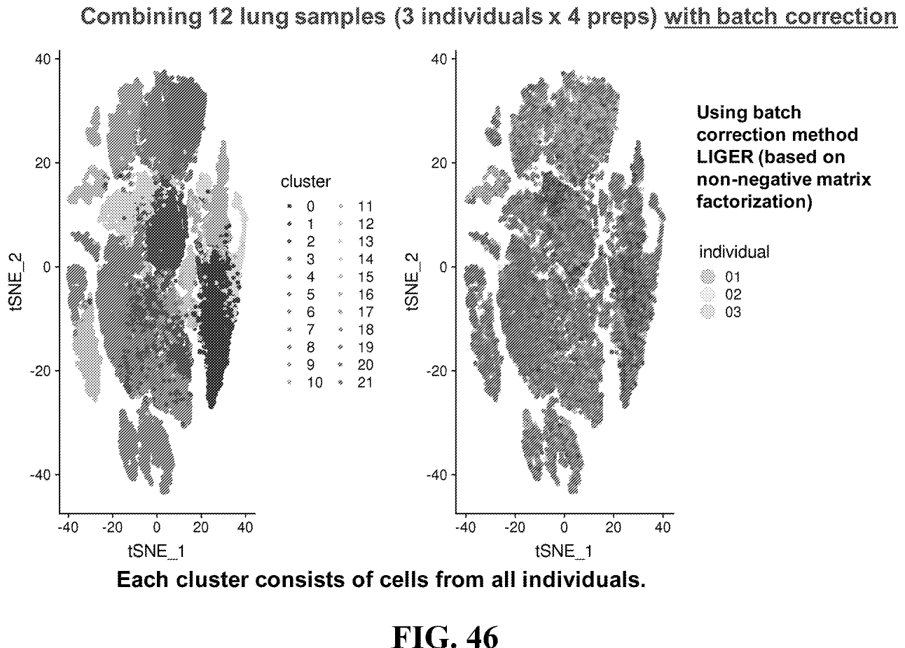

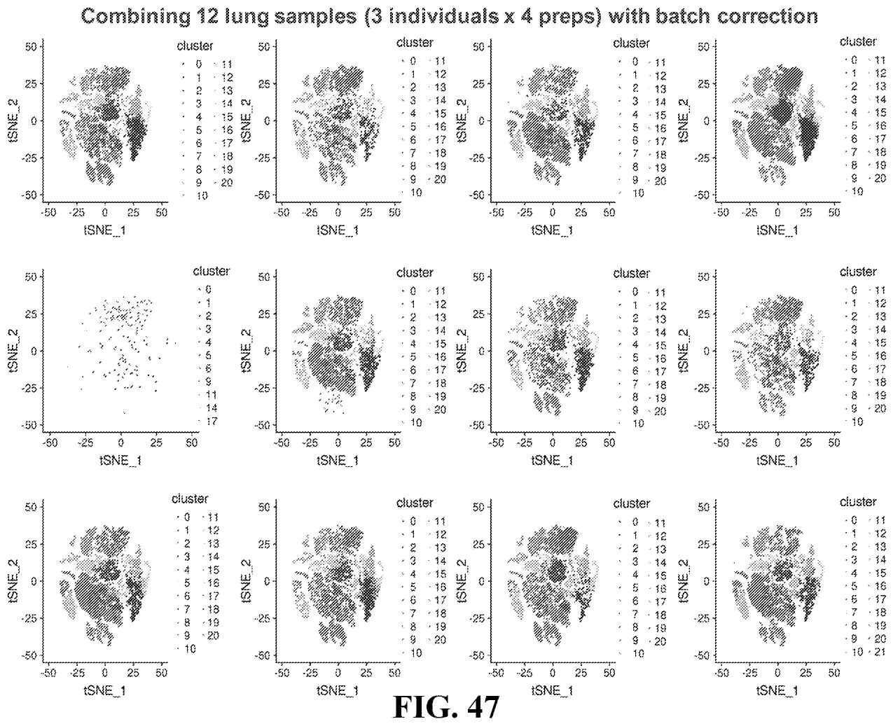

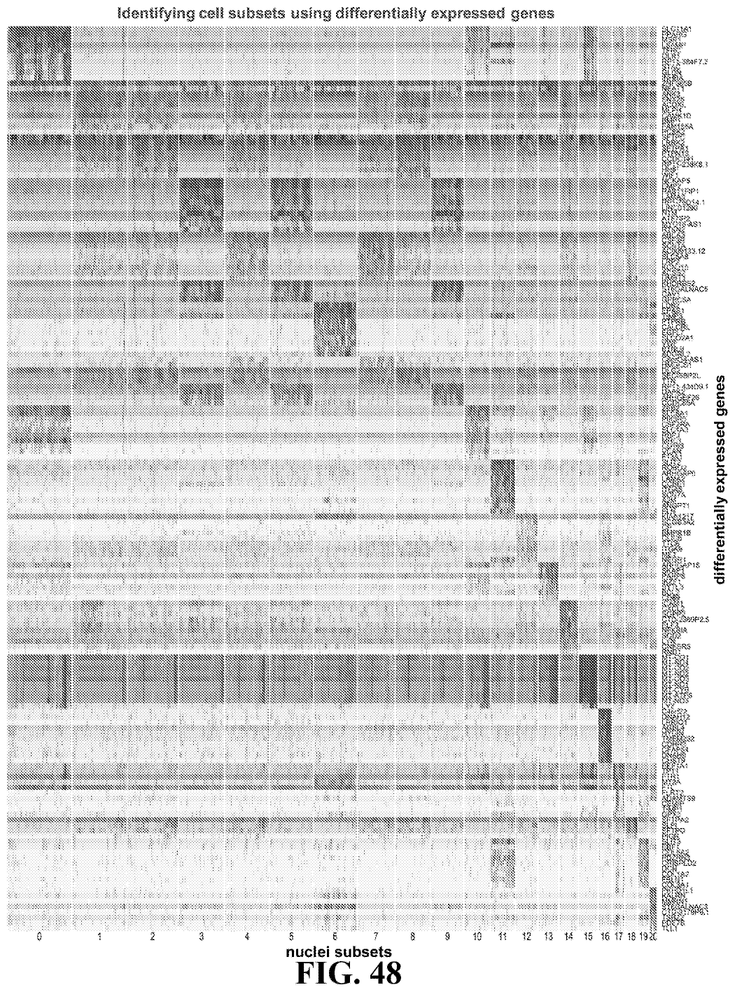

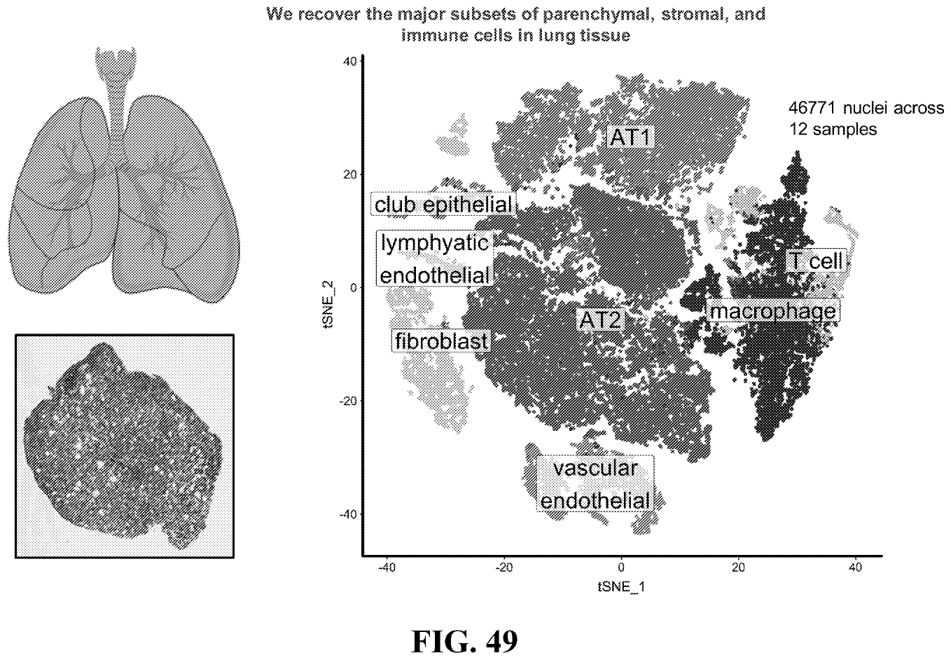

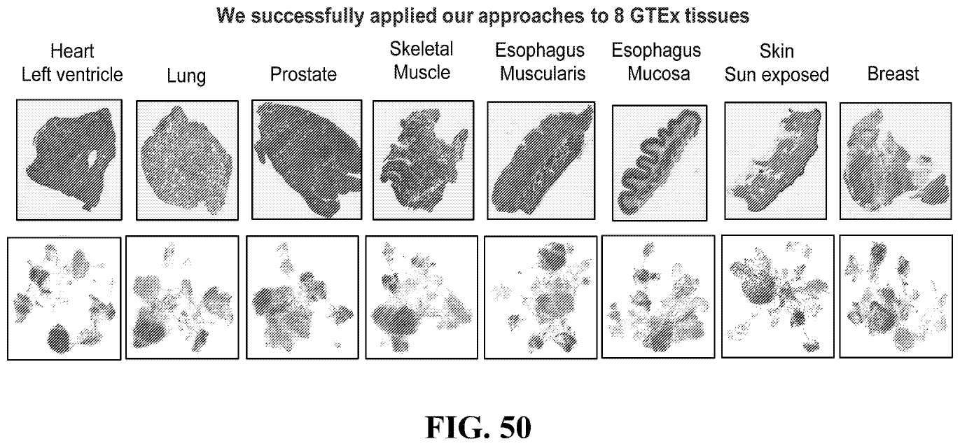

Applicants determined that combining samples increases the power to detect cell subsets, but requires performing batch corrections (FIG. 45). The tSNE plots show that cells cluster by the individuals they came from without using batch correction. Applicants show that using batch correction allows for nuclei to cluster by cell type (FIG. 46). Applicants used 3 different batch correction methods: COMBAT, CCA, and LIGER. Applicants were able to identify corresponding cell subsets while not over-correcting and losing biological state information. Applicants can demultiplex the 12 samples to produce 12 individual tSNE plots (FIG. 47). The nuclei subsets are consistent across the 12 tSNE plots. Applicants identified cell subsets using differentially expressed genes (FIG. 48). Applicants also identified cell subsets using genes curated from the literature. For example, PTPRC (CD45) is a marker for lymphocytes, CD163 is a marker for macrophages, AGER, PDPN and HOPX are markers for Alveolar Type I cells, SFTPB and SFTPC are markers for Alveolar Type II cells, KRT5, TP63 and KRT14 are markers for basal epithelial (CD271+) cells, FOXJ1, TUBA1A and CDHR3 are markers for ciliated epithelial cells, and BPIFA1, SCGB1A1, SCGB3A1 and SCGB3A2 are markers for club epithelial cells. Applicants recovered the major subsets of parenchymal, stromal, and immune cells in lung tissue (FIG. 49). The methods also were able to be applied to 8 GTEx tissues (FIG. 50).

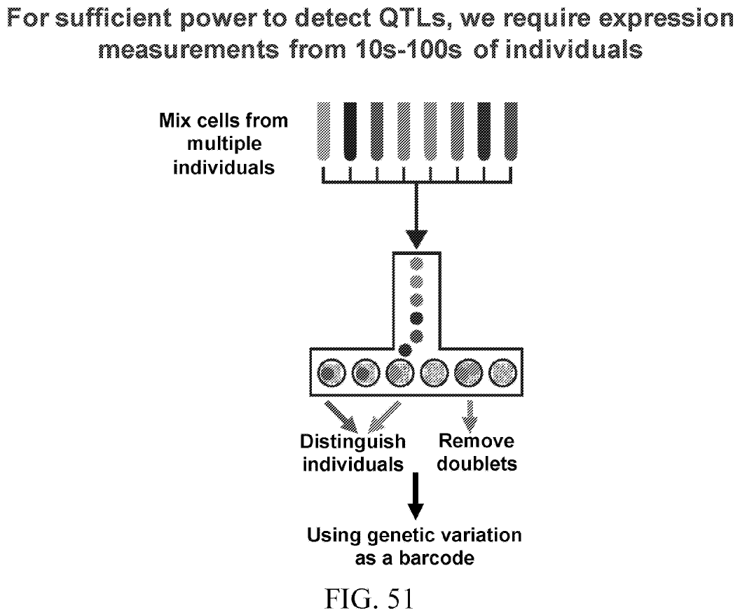

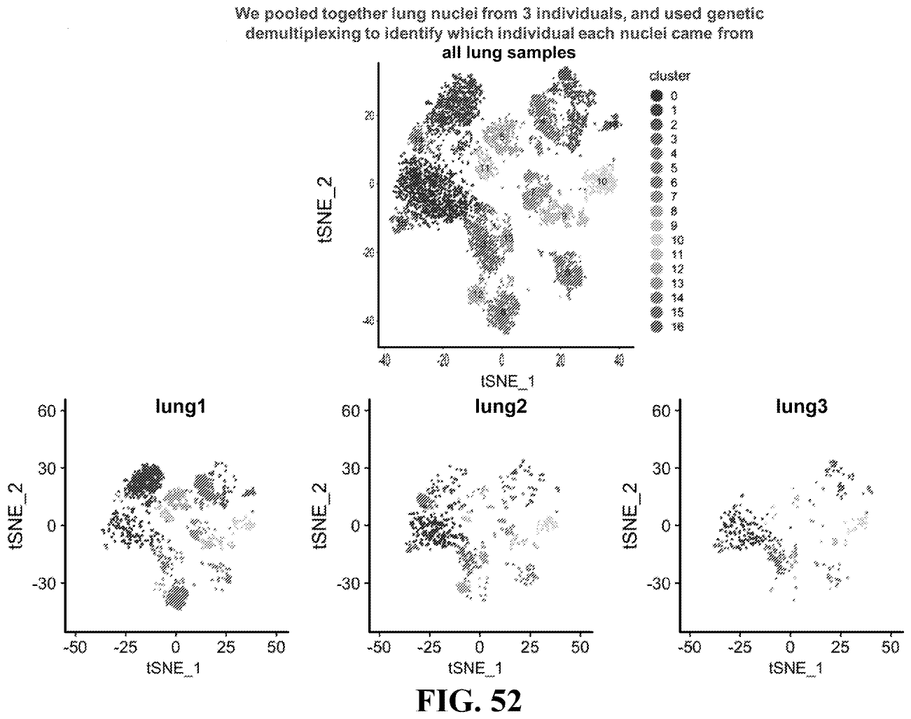

Applicants also determined methods for detecting QTLs (FIG. 51). Applicants determined that for sufficient power to detect QTLs, expression measurements from 10-100s of individuals was required. A quantitative trait locus (QTL) is a region of DNA which is associated with a particular phenotypic trait, which varies in degree and which can be attributed to polygenic effects, i.e., the product of two or more genes, and their environment. Rather than loading each individual on a separate 10× channel (10× Genomics), the samples are mixed together at high concentration. Cord blood from 8 individuals with sequenced genomes is mixed with cells from all 8 individuals and processed together. Droplet-based cell isolation is used. Applicants can distinguish individuals using their sequenced SNPs and remove doublets based on cells or nuclei having SNPs from multiple individuals. Batch effects from cells being encapsulated in droplets can be controlled for.

Applicants show genetic demultiplexing to identify which individual each nuclei came from using lung nuclei pooled from 3 individuals. Applicants pooled three different samples and ran them on the same 10× channel.

### REFERENCES

1. 1. B. B. Yoo, S. K. Mazmanian, The Enteric Network: Interactions between the Immune and Nervous Systems of the Gut. Immunity. 46, 910-926 (2017).
2. J. B. Furness, The enteric nervous system and neurogastroenterology. Nat. Rev. Gastroenterol. Hepatol. 9, 286-294 (2012).
3. V. Sasselli, V. Pachnis, A. J. Burns, The enteric nervous system. Dev. Biol. 366, 64-73 (2012).
4. C. E. Bernard et al., Effect of age on the enteric nervous system of the human colon. Neurogastroenterol. Motif. 21, 746-e46 (2009).
5. M. Li et al., Integrative functional genomic analysis of human brain development and neuropsychiatric risks. Science. 362 (2018), doi :10.1126/science. aat7615.
6. L. A. Scheving, Biological clocks and the digestive system. Gastroenterology. 119, 536-549 (2000).
7. R. De Giorgio et al., Enteric neuropathies: Yesterday, Today and Tomorrow. Adv. Exp. Med. Biol. 891, 123-133 (2016).
8. M. Pesce, O. Borrelli, E. Saliakellis, N. Thapar, Gastrointestinal Neuropathies: New Insights and Emerging Therapies. Gastroenterol. Clin. North Am. 47, 877-894 (2018).
9. C. S. N. Klose et al., The neuropeptide neuromedin U stimulates innate lymphoid cells and type 2 inflammation. Nature. 549, 282-286 (2017).
10. D. Knights, K. G. Lassen, R. J. Xavier, Advances in inflammatory bowel disease pathogenesis: linking host genetics and the microbiome. Gut. 62, 1505-1510 (2013).
11. V. Chaidez, R. L. Hansen, I. Hertz-Picciotto, Gastrointestinal problems in children with autism, developmental delays or typical development. J. Autism Dev. Disord. 44, 1117-1127 (2014).
12. R. F. Pfeiffer, Gastrointestinal dysfunction in Parkinson's disease. Lancet Neurol. 2, 107-116 (2003).
13. J. R. Grider, Neurotransmitters mediating the intestinal peristaltic reflex in the mouse. J. Pharmacol. Exp. Ther. 307, 460-467 (2003).
14. A. B. Rosenberg et al., Single-cell profiling of the developing mouse brain and spinal cord with split-pool barcoding. Science. 360, 176-182 (2018).
15. A. Sathyamurthy et al., Massively Parallel Single Nucleus Transcriptional Profiling Defines Spinal Cord Neurons and Their Activity during Behavior. Cell Rep. 22, 2216-2225 (2018).
16. N. Habib et al., Massively parallel single-nucleus RNA-seq with DroNc-seq. Nat. Methods. 14, 955-958 (2017).
17. N. Habib et al., Div-Seq: Single-nucleus RNA-Seq reveals dynamics of rare adult newborn neurons. Science. 353, 925-928 (2016).
18. B. B. Lake et al., Neuronal subtypes and diversity revealed by single-nucleus RNA sequencing of the human brain. Science. 352, 1586-1590 (2016).
19. A. E. Lewis, H. N. Vasudevan, A. K. O'Neill, P. Soriano, J. O. Bush, The widely used Wnt1-Cre transgene causes developmental phenotypes by ectopic activation of Wnt signaling. Dev. Biol. 379, 229-234 (2013).
20. T. Matsuoka et al., Neural crest origins of the neck and shoulder. Nature. 436, 347-355 (2005).
21. A. Zeisel et al., Molecular Architecture of the Mouse Nervous System. Cell. 174, 999-1014.e22 (2018).
22. R. Lasrado et al., Lineage-dependent spatial and functional organization of the mammalian enteric nervous system. Science. 356, 722-726 (2017).
23. C. B. Wiese, N. Fleming, D. P. Buehler, E. M. Southard-Smith, A Uchl1-Histone2BmCherry:GFP-gpi BAC transgene for imaging neuronal progenitors. Genesis. 51, 852-861 (2013).
24. A. Mo et al., Epigenomic Signatures of Neuronal Diversity in the Mammalian Brain. Neuron. 86, 1369-1384 (2015).
25. C. Simon, H. Lickert, M. Götz, L. Dimou, Sox10-iCreERT2: a mouse line to inducibly trace the neural crest and oligodendrocyte lineage. Genesis. 50, 506-515 (2012).
26. A. J. Prunuske, K. S. Ullman, The nuclear envelope: form and reformation. Curr. Opin. Cell Biol. 18, 108-116 (2006).
27. P. Betancur, M. Bronner-Fraser, T. Sauka-Spengler, Genomic code for Sox10 activation reveals a key regulatory enhancer for cranial neural crest. Proc. Natl. Acad. Sci. U.S.A. 107, 3570-3575 (2010).
28. A. Herrero, J. M. Duhart, M. F. Ceriani, Neuronal and Glial Clocks Underlying Structural Remodeling of Pacemaker Neurons in. Front. Physiol. 8, 918 (2017).
29. B. M. Assas, J. I. Pennock, J. A. Miyan, Calcitonin gene-related peptide is a key neurotransmitter in the neuro-immune axis. Front. Neurosci. 8, 23 (2014).
30. I. S. Junttila, Tuning the Cytokine Responses: An Update on Interleukin (IL)-4 and IL-13 Receptor Complexes. Front. Immunol. 9, 888 (2018).
31. J. Yan, H. Wang, Y. Liu, C. Shao, Analysis of gene regulatory networks in the mammalian circadian rhythm. PLoS Comput. Biol. 4, e1000193 (2008).
32. N. J. Spencer, D. J. Keating, Is There a Role for Endogenous 5-HT in Gastrointestinal Motility? How Recent Studies Have Changed Our Understanding. Adv. Exp. Med. Biol. 891, 113-122 (2016).
33. C. Alcaino, G. Farrugia, A. Beyder, Mechanosensitive Piezo Channels in the Gastrointestinal Tract. Curr. Top. Membr. 79, 219-244 (2017).
34. G. S. Cottrell et al., Localization of calcitonin receptor-like receptor (CLR) and receptor activity-modifying protein 1 (RAMP1) in human gastrointestinal tract. Peptides. 35, 202-211 (2012).
35. A. L. Haber et al., A single-cell survey of the small intestinal epithelium. Nature. 551, 333-339 (2017).
36. J. B. Furness, C. Jones, K. Nurgali, N. Clerc, Intrinsic primary afferent neurons and nerve circuits within the intestine. Prog. Neurobiol. 72, 143-164 (2004).
37. Y.-B. Yu et al., Brain-derived neurotrophic factor contributes to abdominal pain in irritable bowel syndrome. Gut. 61, 685-694 (2012).
38. J. H. Szurszewski, L. G. Ermilov, S. M. Miller, Prevertebral ganglia and intestinofugal afferent neurones. Gut. 51 Suppl 1, i6-10 (2002).
39. V. D. Corleto, Somatostatin and the gastrointestinal tract. Curr. Opin. Endocrinol. Diabetes Obes. 17, 63-68 (2010).
40. A.-P. G. Haramis et al., De novo crypt formation and juvenile polyposis on BMP inhibition in mouse intestine. Science. 303, 1684-1686 (2004).
41. N. Satoh-Takayama et al., IL-7 and IL-15 independently program the differentiation of intestinal CD3-NKp46+ cell subsets from Id2-dependent precursors. J. Exp. Med. 207, 273-280 (2010).
42. S. Degan, G. Y. Lopez, K. Kevill, M. E. Sunday, Gastrin-releasing peptide, immune responses, and lung disease. Ann. N. Y. Acad. Sci. 1144, 136-147 (2008).
43. J. B. Furness, Types of neurons in the enteric nervous system. J. Auton. Nerv. Syst. 81, 87-96 (2000).
44. D. J. Drucker, B. Yusta, Physiology and pharmacology of the enteroendocrine hormone glucagon-like peptide-2. Annu. Rev. Physiol. 76, 561-583 (2014).
45. S. Afroze et al., The physiological roles of secretin and its receptor. Ann Transl Med. 1, 29 (2013).
46. D. Artis, H. Spits, The biology of innate lymphoid cells. Nature. 517, 293-301 (2015).
47. A. Cianferoni, J. Spergel, The importance of TSLP in allergic disease and its role as a potential therapeutic target. Expert Rev. Clin. Immunol. 10, 1463-1474 (2014).
48. S. A. G. Black, R. J. Rylett, Choline transporter CHT regulation and function in cholinergic neurons. Cent. Nerv. Syst. Agents Med. Chem. 12, 114-121 (2012).
49. C. Legay, Why so many forms of acetylcholinesterase? Microsc. Res. Tech. 49, 56-72 (2000).
50. O. A. Al-Shboul, The importance of interstitial cells of caj al in the gastrointestinal tract. Saudi J. Gastroenterol. 19, 3-15 (2013).
51. P. J. Gomez-Pinilla et al., Anol is a selective marker of interstitial cells of Cajal in the human and mouse gastrointestinal tract. Am. J. Physiol. Gastrointest. Liver Physiol. 296, G1370-81 (2009).
52. P. König, D. Groneberg, R. Jäger, A. Friebe, NO-sensitive guanylyl cyclase is expressed in pericytes but absent from endothelial cells in the murine lung. BMC Pharmacol. 11 (2011), doi:10.1186/1471-2210-11-s1-p38.
53. D. Groneberg, P. König, D. Koesling, A. Friebe, Nitric oxide-sensitive guanylyl cyclase is dispensable for nitrergic signaling and gut motility in mouse intestinal smooth muscle. Gastroenterology. 140, 1608-1617 (2011).
54. C. S. Smillie et al., Rewiring of the cellular and inter-cellular landscape of the human colon during ulcerative colitis. bioRxiv (2018), p. 455451.
55. W. G. Lima, G. H. Marques-Oliveira, T. M. da Silva, V. E. Chaves, Role of calcitonin gene-related peptide in energy metabolism. Endocrine. 58, 3-13 (2017).
56. K. Loh, H. Herzog, Y.-C. Shi, Regulation of energy homeostasis by the NPY system. Trends Endocrinol. Metab. 26, 125-135 (2015).
57. V. Athie-Morales, H. H. Smits, D. A. Cantrell, C. M. U. Hilkens, Sustained IL-12 signaling is required for Th1 development. J. Immunol. 172, 61-69 (2004).
58. D. Chang et al., A meta-analysis of genome-wide association studies identifies 17 new Parkinson's disease risk loci. Nat. Genet. 49, 1511-1516 (2017).
59. S. J. Sanders et al., Insights into Autism Spectrum Disorder Genomic Architecture and Biology from 71 Risk Loci. Neuron. 87, 1215-1233 (2015).
60. N. Bondurand, E. M. Southard-Smith, Mouse models of Hirschsprung disease and other developmental disorders of the enteric nervous system: Old and new players. Dev. Biol. 417, 139-157 (2016).
61. P. Pla, L. Lame, Involvement of endothelin receptors in normal and pathological development of neural crest cells. Int. J Dev. Biol. 47, 315-325 (2003).
62. J. Ischia, O. Patel, A. Shulkes, G. S. Baldwin, Gastrin-releasing peptide: different forms, different functions. Biofactors. 35, 69-75 (2009).
63. L. Tomas-Roca et al., De novo mutations in PLXND1 and REV3L cause Möbius syndrome. Nat. Commun. 6, 7199 (2015).
64. K. J. Gross, C. Pothoulakis, Role of neuropeptides in inflammatory bowel disease. Inflamm. Bowel Dis. 13, 918-932 (2007).
65. F. R. Cagampang, K. D. Bruce, The role of the circadian clock system in nutrition and metabolism. Br. J. Nutr. 108, 381-392 (2012).
66. T. Vatanen et al., Variation in Microbiome LPS Immunogenicity Contributes to Autoimmunity in Humans. Cell. 165, 1551 (2016).
67. V. Gopalakrishnan, B. A. Helmink, C. N. Spencer, A. Reuben, J. A. Wargo, The Influence of the Gut Microbiome on Cancer, Immunity, and Cancer Immunotherapy. Cancer Cell. 33, 570-580 (2018).
68. A. Deczkowska, M. Schwartz, Targeting neuro-immune communication in neurodegeneration: Challenges and opportunities. J. Exp. Med. 215, 2702-2704 (2018).
69. A. Mo et al., Epigenomic Signatures of Neuronal Diversity in the Mammalian Brain. Neuron. 86, 1369-1384 (2015).
70. C. B. Wiese, N. Fleming, D. P. Buehler, E. M. Southard-Smith, A Uchl1-Histone2BmCherry:GFP-gpi BAC transgene for imaging neuronal progenitors. Genesis. 51, 852-861 (2013).
71. E. Drokhlyansky et al., The brain parenchyma has a type I interferon response that can limit virus spread. Proc. Natl. Acad. Sci. U.S.A. 114, E95-E104 (2017).
72. T. Pietzsch, S. Preibisch, P. Tomancák, S. Saalfeld, ImgLib2-generic image processing in Java. Bioinformatics. 28, 3009-3011 (2012).
73. J. Schindelin et al., Fiji: an open-source platform for biological-image analysis. Nat. Methods. 9, 676-682 (2012).
74. J. Schindelin, C. T. Rueden, M. C. Hiner, K. W. Eliceiri, The ImageJ ecosystem: An open platform for biomedical image analysis. Mol. Reprod. Dev. 82, 518-529 (2015).
75. C. A. Schneider, W. S. Rasband, K. W. Eliceiri, NIH Image to ImageJ: 25 years of image analysis. Nat. Methods. 9, 671-675 (2012).
76. M. Linkert et al., Metadata matters: access to image data in the real world. J. Cell Biol. 189, 777-782 (2010).
77. N. Habib et al., Div-Seq: Single-nucleus RNA-Seq reveals dynamics of rare adult newborn neurons. Science. 353, 925-928 (2016).
78. N. Habib et al., Massively parallel single-nucleus RNA-seq with DroNc-seq. Nat. Methods. 14, 955-958 (2017).
79. K. Shekhar et al., Comprehensive Classification of Retinal Bipolar Neurons by Single-Cell Transcriptomics. Cell. 166, 1308-1323 . e30 (2016).
80. B. Li, C. N. Dewey, RSEM: accurate transcript quantification from RNA-Seq data with or without a reference genome. BMC Bioinformatics. 12, 323 (2011).
81. A. L. Haber et al., A single-cell survey of the small intestinal epithelium. Nature. 551, 333-339 (2017).
82. A. Butler, P. Hoffman, P. Smibert, E. Papalexi, R. Satija, Integrating single-cell transcriptomic data across different conditions, technologies, and species. Nat. Biotechnol. 36, 411 (2018).
83. A. Schuchardt, V. D'Agati, V. Pachnis, F. Costantini, Renal agenesis and hypodysplasia in ret-k-mutant mice result from defects in ureteric bud development. Development. 122, 1919-1929 (1996).
84. J. Rossi et al., Retarded growth and deficits in the enteric and parasympathetic nervous system in mice lacking GFR alpha2, a functional neurturin receptor. Neuron. 22, 243-252 (1999).
85. S. J. Brookes, P. A. Steele, M. Costa, Identification and immunohistochemistry of cholinergic and non-cholinergic circular muscle motor neurons in the guinea-pig small intestine. Neuroscience. 42, 863-878 (1991).
86. S. J. Brookes, P. A. Steele, M. Costa, Calretinin immunoreactivity in cholinergic motor neurones, interneurones and vasomotor neurones in the guinea-pig small intestine. Cell Tissue Res. 263, 471-481 (1991).
87. J. R. Grider, Neurotransmitters mediating the intestinal peristaltic reflex in the mouse. J. Pharmacol. Exp. Ther. 307, 460-467 (2003).
88. Q. Sang, H. M. Young, Chemical coding of neurons in the myenteric plexus and external muscle of the small and large intestine of the mouse. Cell Tissue Res. 284, 39-53 (1996).
89. H. M. Young, J. B. Furness, J. M. Povey, Analysis of connections between nitric oxide synthase neurons in the myenteric plexus of the guinea-pig small intestine. J. Neurocytol. 24, 257-263 (1995).
90. J. B. Furness, The enteric nervous system and neurogastroenterology. Nat. Rev. Gastroenterol. Hepatol. 9, 286-294 (2012).
91. J. R. Grider, Interplay of somatostatin, opioid, and GABA neurons in the regulation of the peristaltic reflex. Am. J. Physiol. 267, G696-701 (1994).
92. J. B. Furness, M. Costa, A. Rökaeus, T. J. McDonald, B. Brooks, Galanin-immunoreactive neurons in the guinea-pig small intestine: their projections and relationships to other enteric neurons. Cell Tissue Res. 250, 607-615 (1987).
93. R. Lasrado et al., Lineage-dependent spatial and functional organization of the mammalian enteric nervous system. Science. 356, 722-726 (2017).
94. Y. Benjamini, Y. Hochberg, Controlling the False Discovery Rate: A Practical and Powerful Approach to Multiple Testing. J. R. Stat. Soc. Series B Stat. Methodol. 57, 289-300 (1995).
95. R. Lopez, J. Regier, M. B. Cole, M. I. Jordan, N. Yosef, Deep generative modeling for single-cell transcriptomics. Nat. Methods. 15, 1053-1058 (2018).
96. J. A. Ramilowski et al., A draft network of ligand-receptor-mediated multicellular signalling in human. Nat. Commun. 6, 7866 (2015).
97. R. Vento-Tormo et al., Single-cell reconstruction of the early maternal-fetal interface in humans. Nature. 563, 347-353 (2018).
98. N. Bondurand, E. M. Southard-Smith, Mouse models of Hirschsprung disease and other developmental disorders of the enteric nervous system: Old and new players. Dev. Biol. 417, 139-157 (2016).
99. C. S. Smillie et al., Rewiring of the cellular and inter-cellular landscape of the human colon during ulcerative colitis (2018), doi:10.1101/455451.
100. S. J. Sanders et al., Insights into Autism Spectrum Disorder Genomic Architecture and Biology from 71 Risk Loci. Neuron. 87, 1215-1233 (2015).
101. D. Chang et al., A meta-analysis of genome-wide association studies identifies 17 new Parkinson's disease risk loci. Nat. Genet. 49, 1511-1516 (2017).
102. T. Matsuoka et al., Neural crest origins of the neck and shoulder. Nature. 436, 347-355 (2005).
103. A. E. Lewis, H. N. Vasudevan, A. K. O'Neill, P. Soriano, J. O. Bush, The widely used Wnt1-Cre transgene causes developmental phenotypes by ectopic activation of Wnt signaling. Dev. Biol. 379, 229-234 (2013).

### Example 22

### A Single-Cell and Single-Nucleus RNA-seq Toolbox for Fresh and Frozen Human Tumors

Single cell RNA-Seq (scRNA-Seq) has transformed the ability to analyze tumors, revealing cell types, states, genetic diversity, and interactions in the complex tumor ecosystem1-6. However, successful scRNA-Seq requires dissociation tailored to the tumor type, and involves enzymatic digestion that can lead to loss of sensitive cells or changes in gene expression. Moreover, obtaining fresh tissue is time-sensitive and requires tight coordination between tissue acquisition and processing teams, posing a challenge in clinical settings. Conversely, single-nucleus RNA-Seq (snRNA-Seq) allows profiling of single nuclei isolated from frozen tissues, decoupling tissue acquisition from immediate sample processing. snRNA-Seq can also handle samples that cannot be successfully dissociated even when fresh, due to size or cell fragility7,8, as well as multiplexed analysis of longitudinal samples from the same individual9. However, nuclei have lower amounts of mRNA compared to cells, and are more challenging to enrich or deplete. Both scRNA-Seq and snRNA-Seq pose experimental challenges when applied to different tumor types, due to distinct cellular composition and extracellular matrix (ECM) in different tumors.

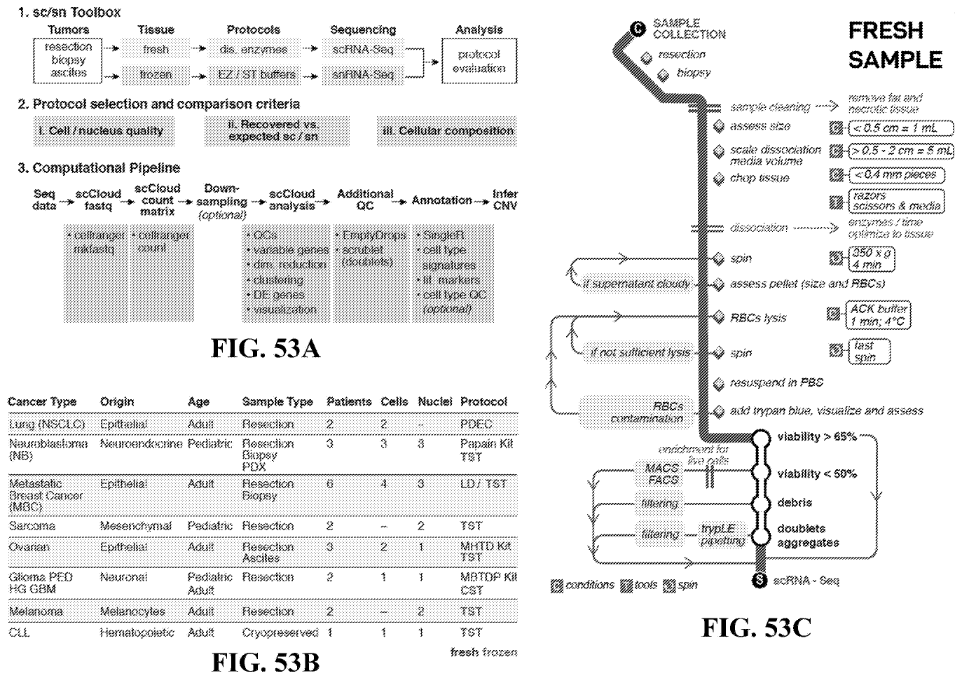

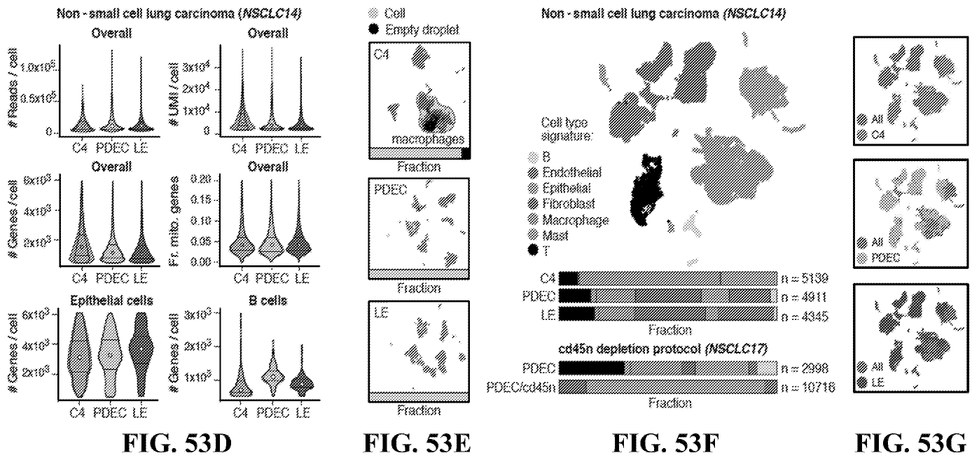

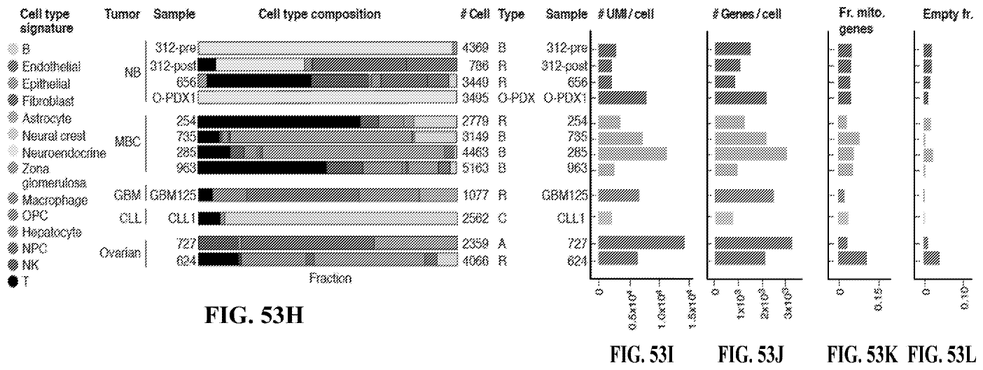

To address these challenges, Applicants developed a systematic toolbox for fresh and frozen tumor processing using single cell (sc) and single nucleus (sn) RNA-Seq, respectively (FIG. 53A). Applicants tested eight tumor types with different tissue characteristics (FIG. 53B), including comparisons of matched fresh and frozen preparations from the same tumor specimen. The tumor types span different cell-of-origin (e.g., epithelial, neuronal), solid and non-solid, patient ages, and transitions (e.g., primary, metastatic, FIG. 53B).

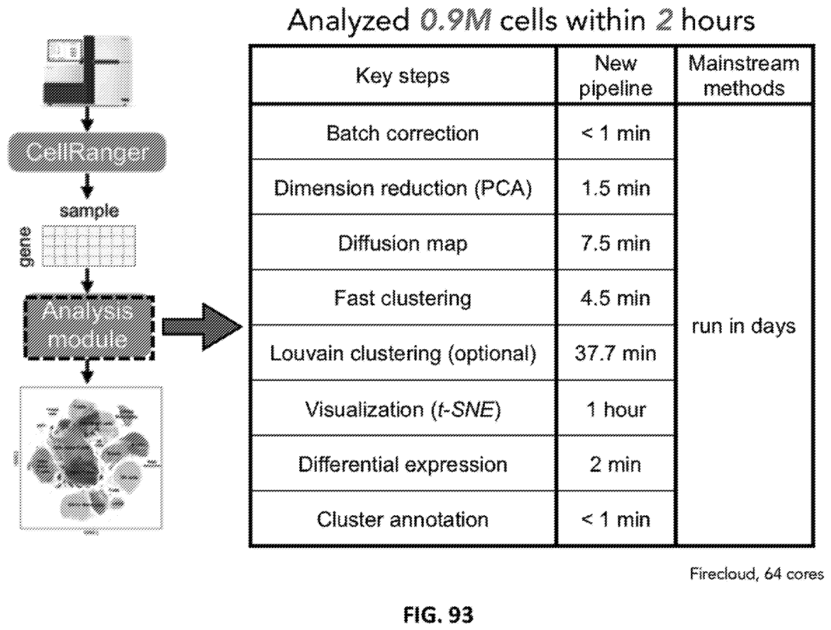

Applicants evaluated and compared protocols based on (i) cell/nucleus quality; (ii) number of recovered vs. expected cells/nuclei; and (iii) cellular composition (FIG. 53A). For “cell/nucleus quality”, Applicants considered both experimental and computational metrics. Experimentally, Applicants measured cell viability (for scRNA-Seq), the extent of doublets or aggregates in the cell/nucleus suspension, and cDNA quality recovered after Whole Transcriptome Amplification (Methods). Computationally, Applicants evaluated the overall number of sequencing reads in a library, the percent of reads mapping to the transcriptome, genome, and intergenic regions, the number of cells/nuclei exceeding a minimal number of genes and unique transcripts (reflected by Unique Molecular Identifiers; UMI), the number of reads, transcripts (UMIs), and genes detected per cell/nucleus, and the percent of UMIs from mitochondrial genes (Methods). For “number of recovered vs. expected cells/nuclei”, Applicants considered the proportion of droplets scored as likely empty (i.e., containing only ambient RNA rather than the RNA from an encapsulated cell¹⁰), and the proportion of doublets¹¹ (Methods). Finally, for “cellular composition”, Applicants considered the diversity of cell types captured, the proportion of cells/nuclei recovered from each subset, and the copy number aberration (CNA) pattern classes that are recovered in malignant cells (Methods). Applicants annotated the malignant cells based on the presence of CNAs (when detectable) and the cell type signature they most closely resembled (Methods). Applicants conducted most data analysis using scCloud, a Cloud based single-cell analysis pipeline¹² (https://github.com/klarman-cell-observatory/scCloud, Methods, FIGS. 53A and 93).

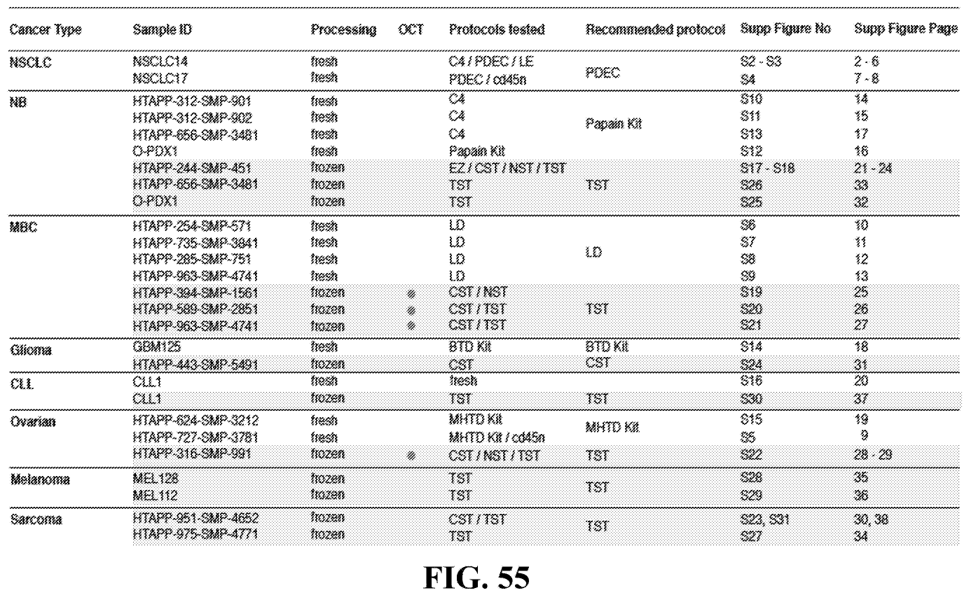

For scRNA-seq, Applicants' toolbox encompasses successful protocols for five types of fresh tumors: non-small cell lung carcinoma (NSCLC), metastatic breast cancer (MBC), ovarian cancer, glioblastoma (GBM), and neuroblastoma, as well as a cryopreserved non-solid, chronic lymphocytic leukemia (CLL) (FIGS. 53B, 55). Applicants constructed workflows that minimize the time interval between removal of the sample from the patient in a clinical setting and its dissociation into cells, to maximize cell viability and preservation of RNA profiles. Applicants determined dissociation conditions for each of the tumor types and constructed specific steps as a decision tree to adjust for differences between types of clinical samples (e.g., size, presence of red blood cells) (FIG. 53C, Methods). To choose the best performing dissociation method, Applicants apportioned large tumor specimens into smaller pieces (˜0.5-2 cm), dissociating each piece with a different protocol.

Applicants selected enzymatic mixtures for processing fresh tissues based on the specific characteristics of each tumor type, such as cell type composition and ECM components, and ultimately recommend the method that sufficiently breaks down the ECM and cell-to-cell adhesions, while minimizing processing time and supporting the cell type diversity in the sample. For example, to break down collagen fibers in breast cancer^{13,14}, Applicants used Liberase™ (Methods), whereas to break down ECM in GBM15 Applicants used papain (cysteine protease). Applicants also included DNase I to digest DNA released from dead cells to decrease viscosity in all dissociation mixtures. Applicants subjected the samples that yielded high quality single cell suspensions to droplet-based scRNA-Seq (Methods).

As an example of the optimization process, consider NSCLC (sample NSCLC14, FIGS. 55-57) where Applicants used three processing protocols: (1) Collagenase 4 [NSCLC-C4]; (2) a mixture of Pronase, Dispase, Elastase, and Collagenases A and 4 [PDEC]; or (3) Liberase™ and Elastase [LE]; each in combination with DNase I and elastase, to break down the elastin fibers found in lung tissue^{16,17} (Methods) (FIGS. 53D-53G; 56, 57). For the other tumor types, Applicants show the application of the recommended protocol out of those tested (FIGS. 53H-53L; 55).

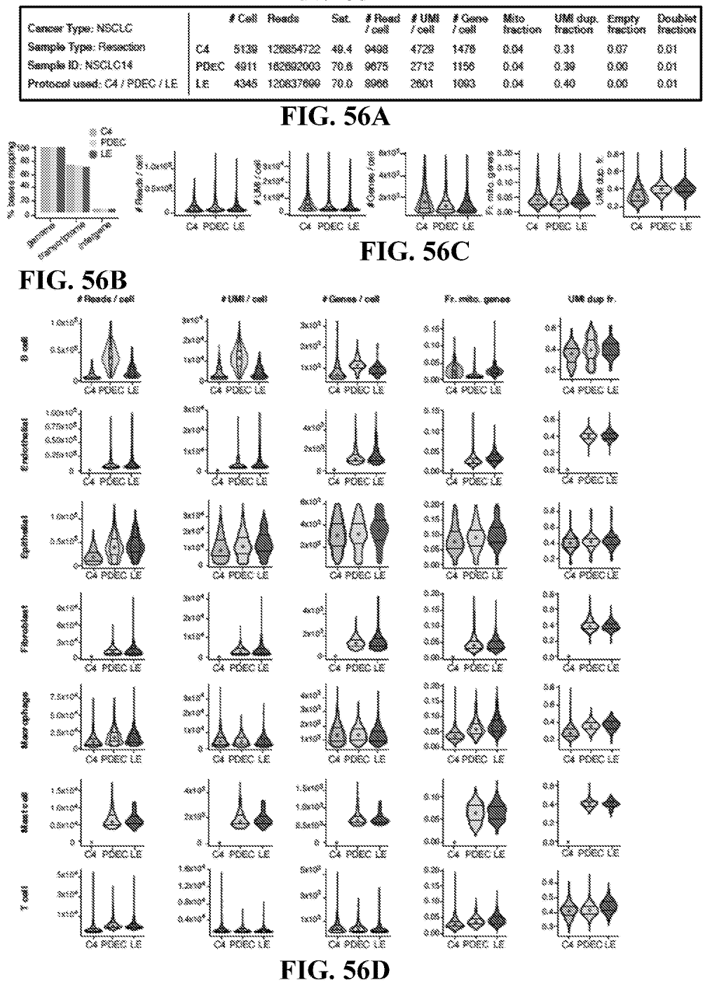

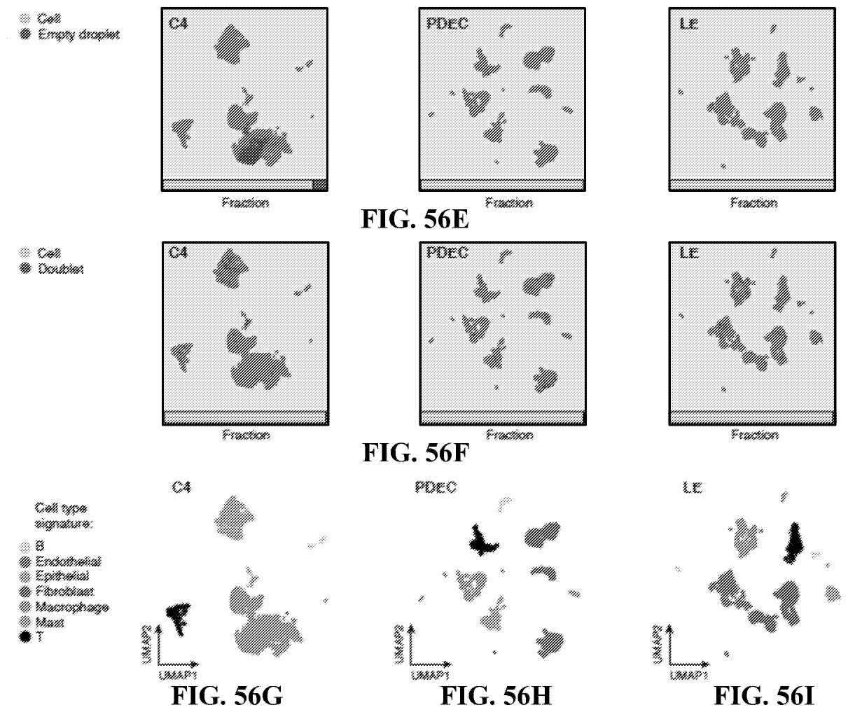

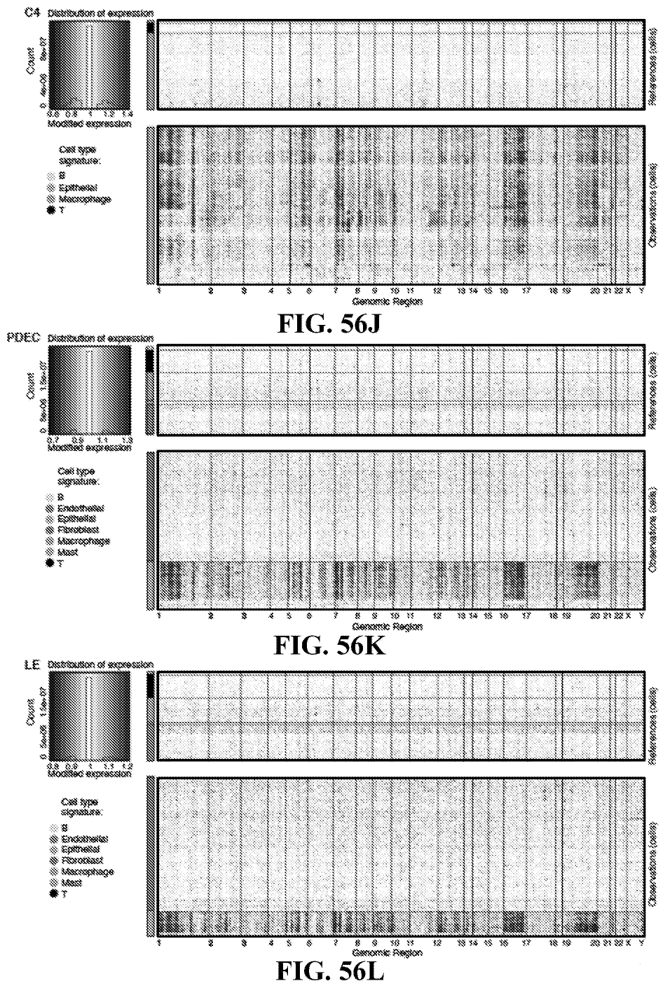

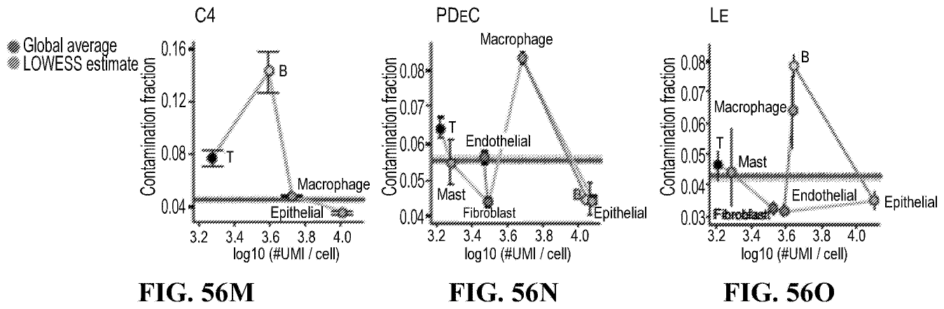

Protocols often performed similarly on standard quality control measures (e.g., number of cells recovered), but differed markedly in cellular diversity or in the fraction of droplets predicted to contain only ambient RNA (“empty drops”)—two evaluation criteria that Applicants prioritized. For example, in the NSCLC resection sample above, all methods yielded a similar number of cells with high-quality expression profiles and similar CNA patterns in malignant cells (FIG. 53D-53G, 56A-56L). However, only the PDEC and LE protocols recovered stromal and endothelial cells (FIG. 53F; 56G), and C4 had a 100-fold higher fraction of drops called as “empty” (7% vs. 0.08% and 0.04% in PDEC and LE, respectively, 56A). The drops designated “empty” in C4 clustered within macrophages (FIGS. 53E; 56E, 56G-56I), the most prevalent cell type, suggesting that these cell barcodes either had lower sequencing saturation or that the sample itself had higher ambient RNA content. While Applicants estimated similarly low levels of ambient RNA18 across the three protocols (FIG. 56M-56O), NCSLC-C4 indeed had lower sequencing saturation and lower reads per cell (FIG. 56A, 56C). Ultimately, taking all of these features into consideration, Applicants recommend the PDEC protocol for processing NSCLC tumor samples.

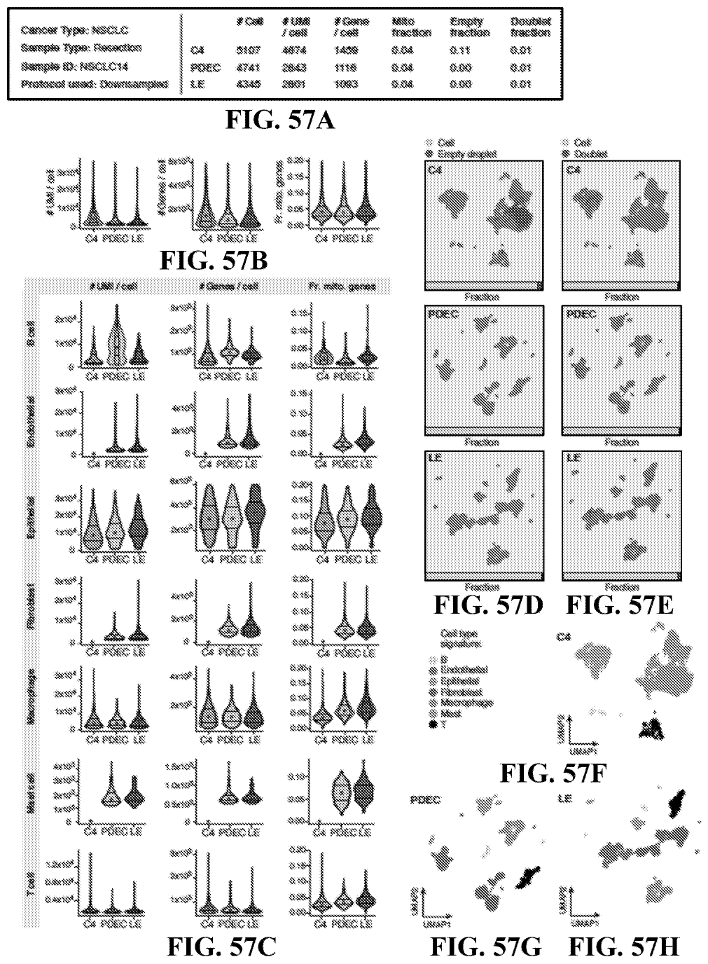

Comparing QC metrics across protocols can be challenging due to differences in cell type recovery and in sequencing depth between preparations, which Applicants controlled for by also evaluating QC metrics within each cell type and down-sampling by total reads across protocols (FIGS. 56D and 57). For example, overall, for the NSCLC sample, C4 had a significantly higher median number of detected genes (P=1.3*10−90 vs. PDEC; 1.4*10−62 vs. LE, Mann-Whitney U test), but within B cells, PDEC had a significantly higher number of genes (P=2*10−15 vs. C4; 2*10−10 vs. LE), whereas within epithelial cells, LE had the highest number (P=5*10−6 vs. C4; 2*10−4 vs. PDEC) (FIGS. 53D; 56D). Because cell type proportions may vary between protocols, and the number of detected genes (and other metrics) varies between cell types, it is important also to assess cell-type specific QCs when choosing a protocol. Down-sampling by total reads did not qualitatively change any of the protocol evaluation metrics (FIG. 57).

Because in some tumor specimens the proportion of malignant cells is relatively low, Applicants further optimized an immune-cell depletion strategy (Methods). Depletion of CD45+ expressing cells circumvents the need for enriching with specific surface markers (e.g., EpCAM), which might otherwise bias the selection of specific cell populations, such as loss of representation of malignant cells undergoing EMT. Depletion applied to another NSCLC tumor sample (NSCLC17) increased the proportion of malignant epithelial cells from 26% in non-depleted scRNA-seq to 82% (FIGS. 53F; 58), and from 1.2% (by FACS) to 29.5% when applied to an ovarian ascites sample (FIG. 53H, sample 727; FIG. 59).

Applicants also successfully applied the scRNA-Seq toolbox to much smaller core biopsy clinical samples. For example, in MBC, we applied the LD (Liberase™ and DNase I) protocol to a resection (HTAPP-254) and a biopsy (HTAPP-735) from lymph node metastases from two patients, yielding similarly successful QCs (FIGS. 53H-53L; 61, 61). The resection and biopsy of the two patients had, however, different cellular compositions (FIG. 53H): a higher proportion of epithelial, endothelial, and fibroblast cells and a lower proportion of T cells in the biopsy compared to the resection. Applicants similarly successfully profiled biopsies of MBC liver metastases (HTAPP-285, HTAPP-963) with the same protocol (FIG. 53H-53L; 62; 63 FIG. 53H). Thus, this protocol can be used across breast cancer metastases from different anatomical metastatic sites.

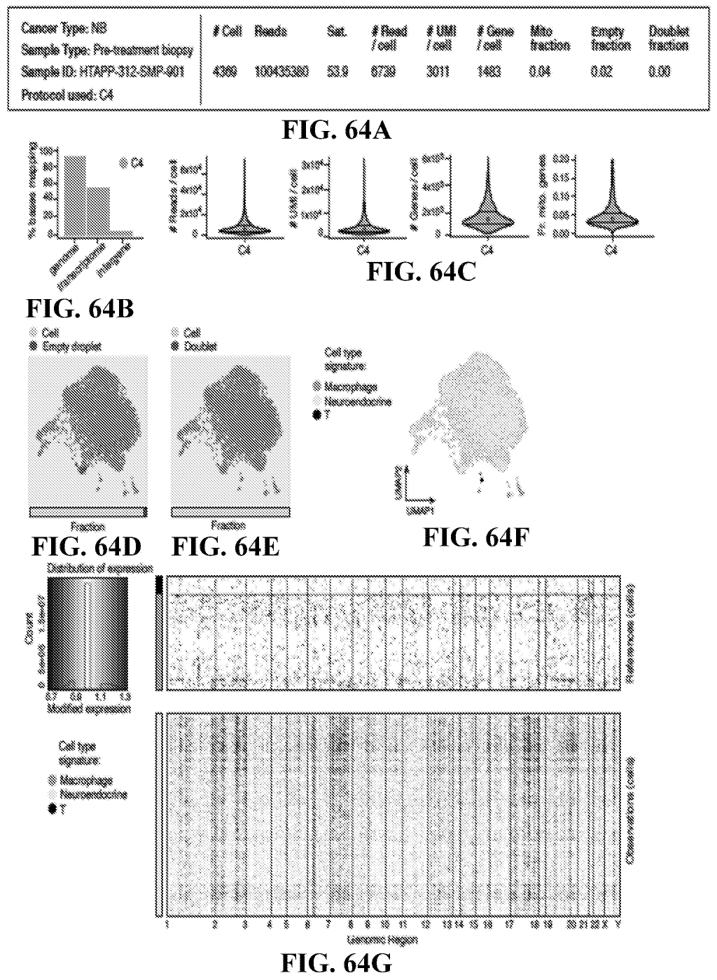

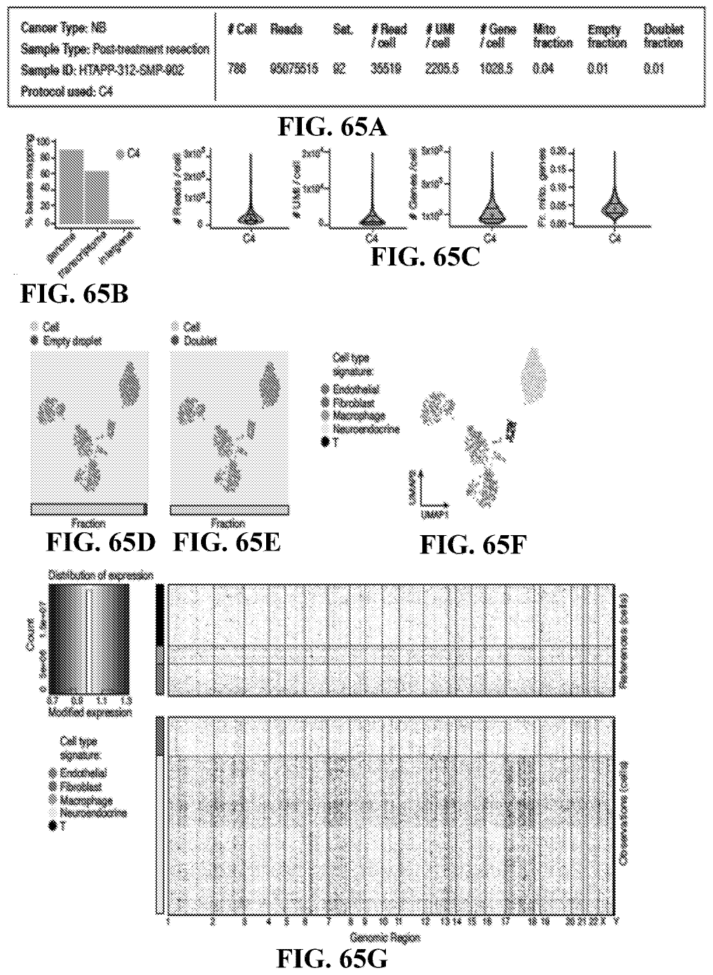

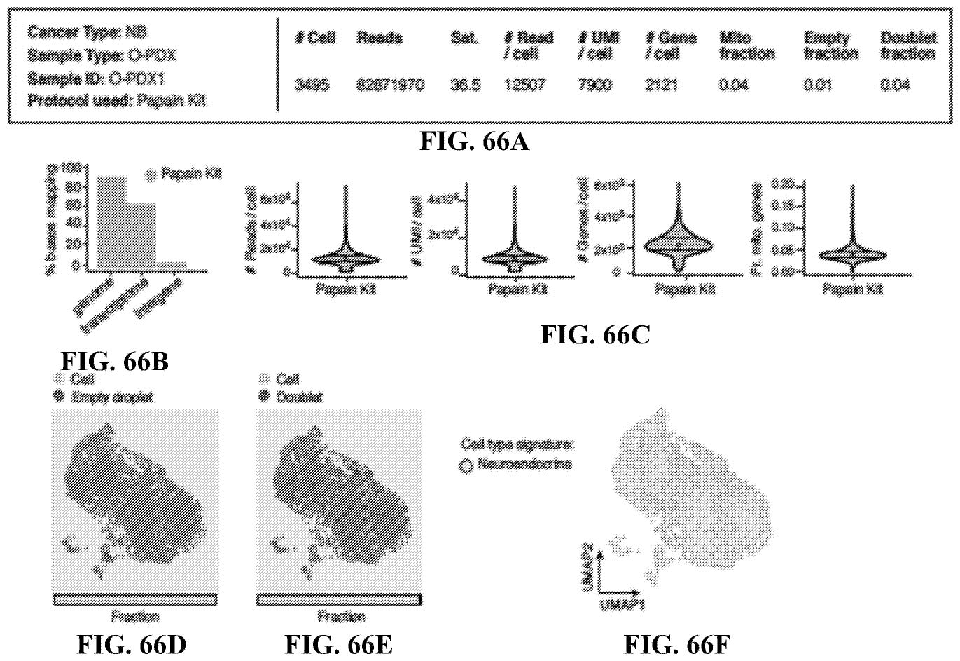

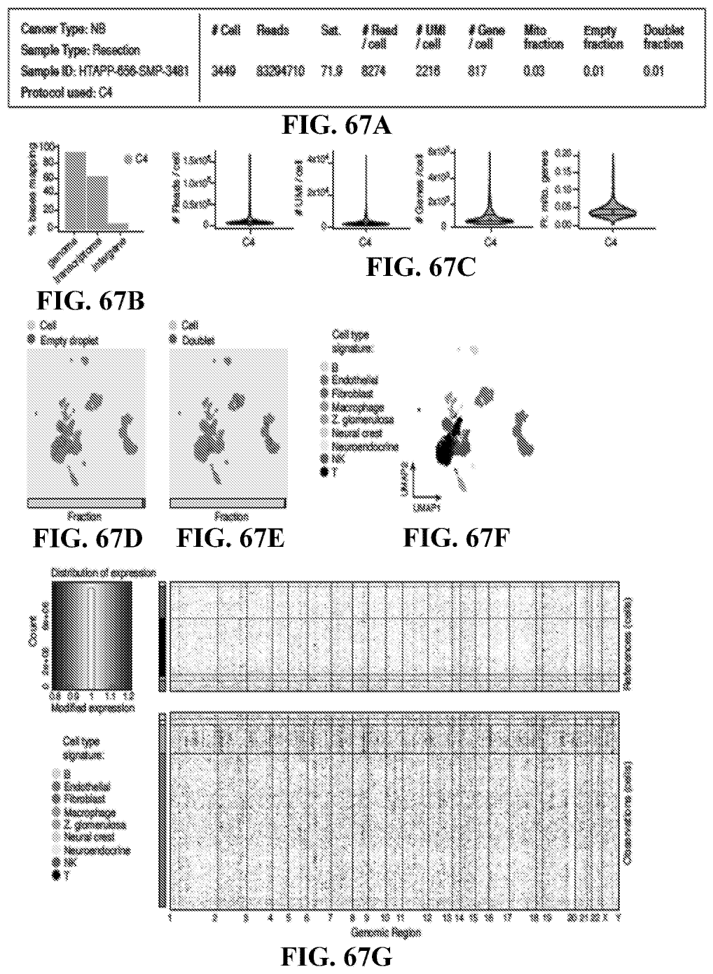

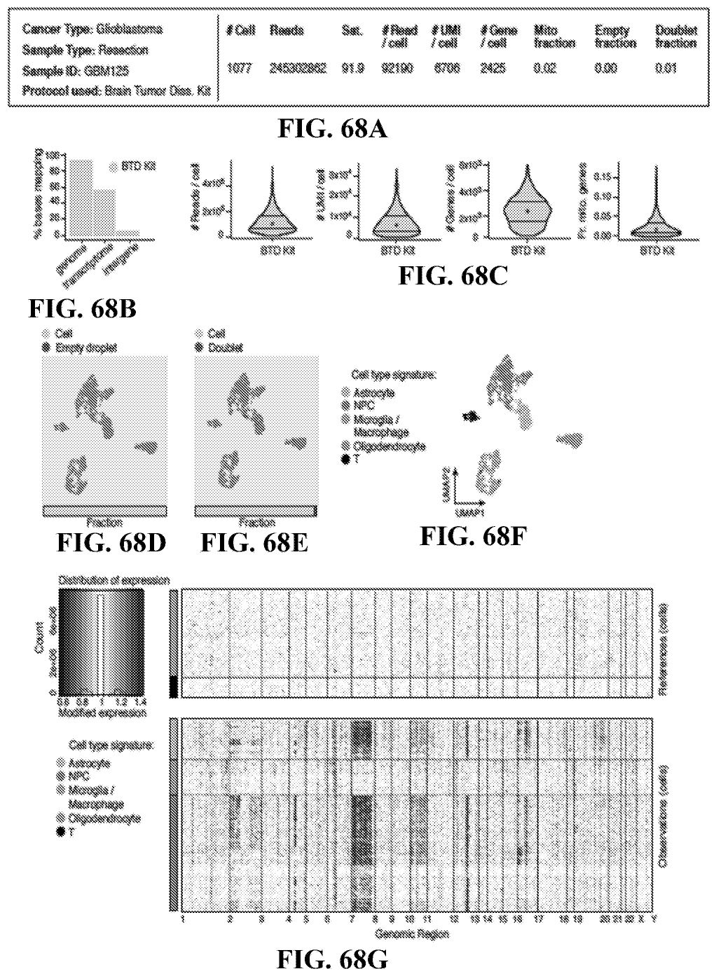

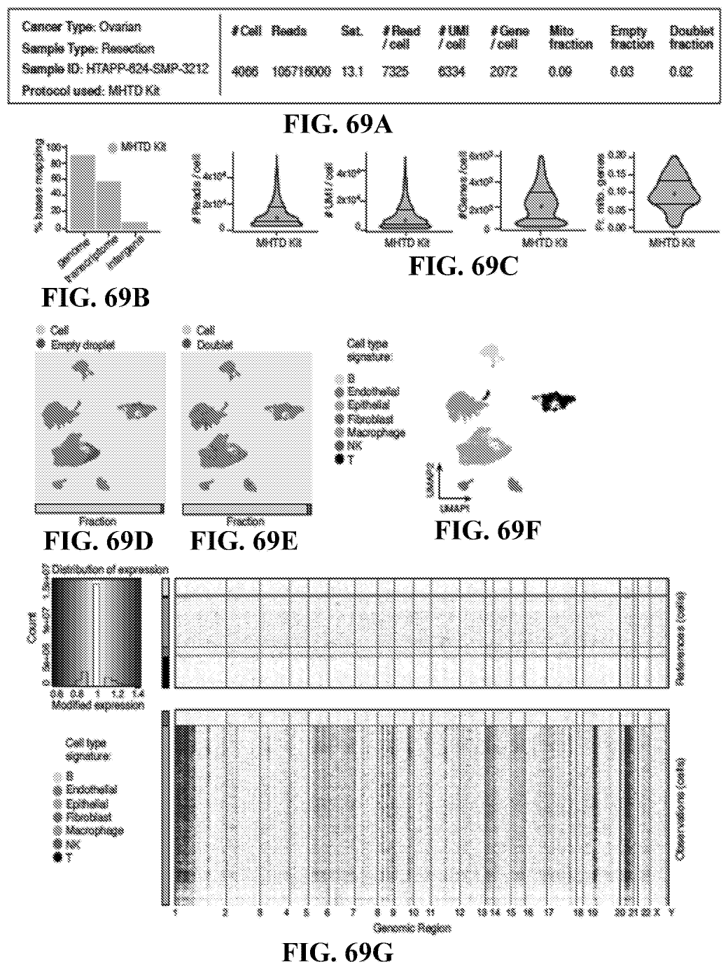

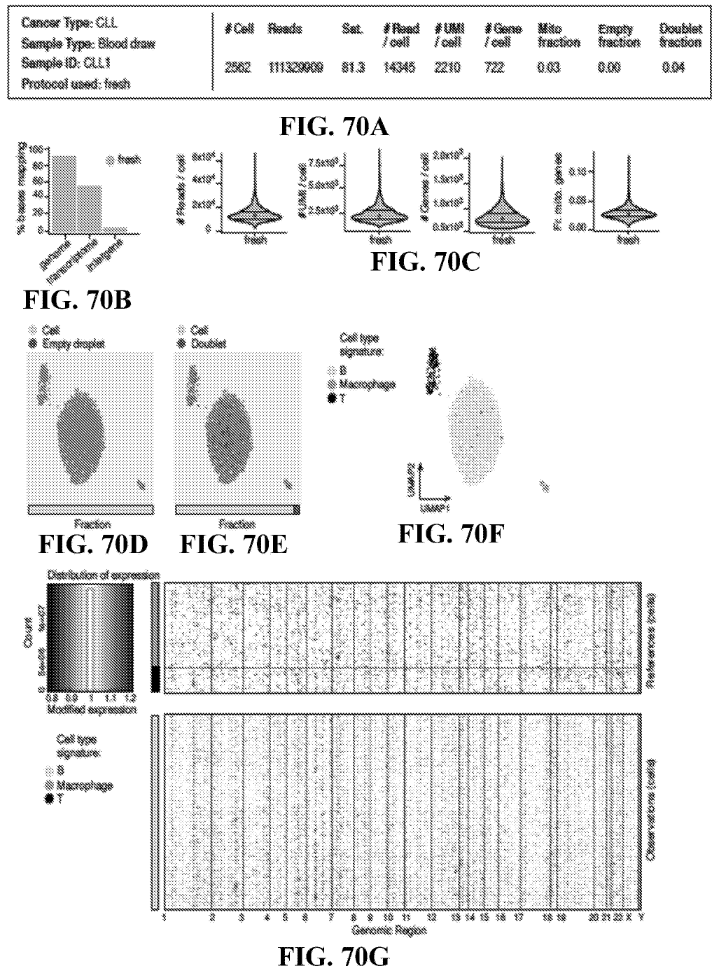

The scRNA-Seq toolbox performs well on samples obtained post-treatment, which can be challenging as a result of cell death and changes in cell type composition with treatment. Applicants demonstrate this in profiling a pre-treatment diagnostic biopsy and post-treatment resection from the same neuroblastoma patient using the NB-C4 protocol (FIG. 53H-53L, HTAPP-312-pre, HTAPP-312-post; FIGS. 64, 65). More cells but of fewer cell types were recovered in the pre-treatment biopsy (4,369 cells: neuroendocrine, T cells, and macrophages) than the post-treatment resection (786 cells: neuroendocrine, T cells, macrophages, as well as endothelial cells, and fibroblasts), consistent with observed post-treatment fibrosis. Applicants tested an additional dissociation protocol in a neuroblastoma orthotopic patient-derived xenograft (O-PDX) sample (O-PDX1)^{19,20}, which is not expected to include non-malignant human cells, and indeed resulted in high quality malignant cell profiles (FIG. 66).

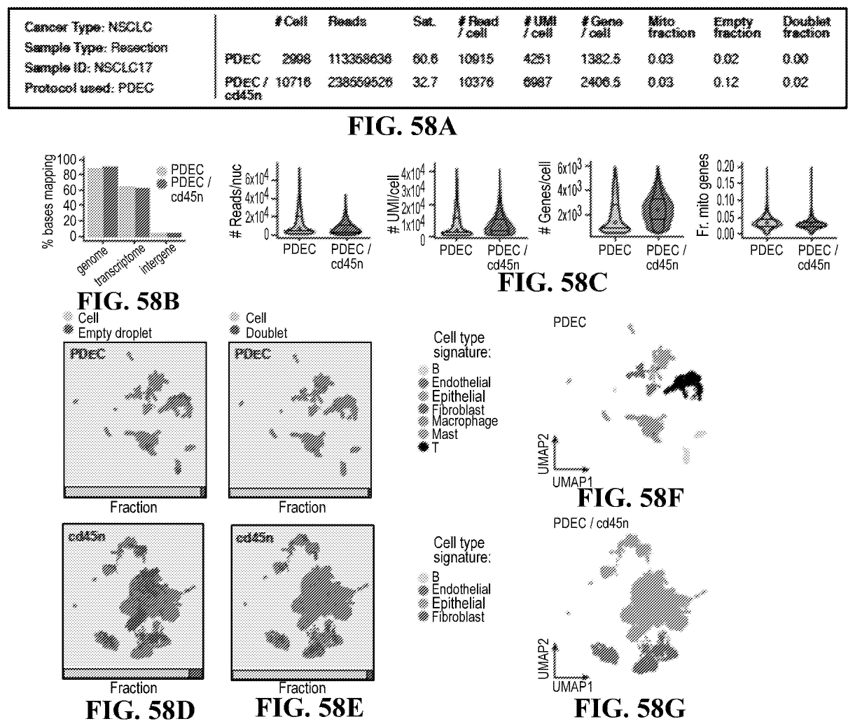

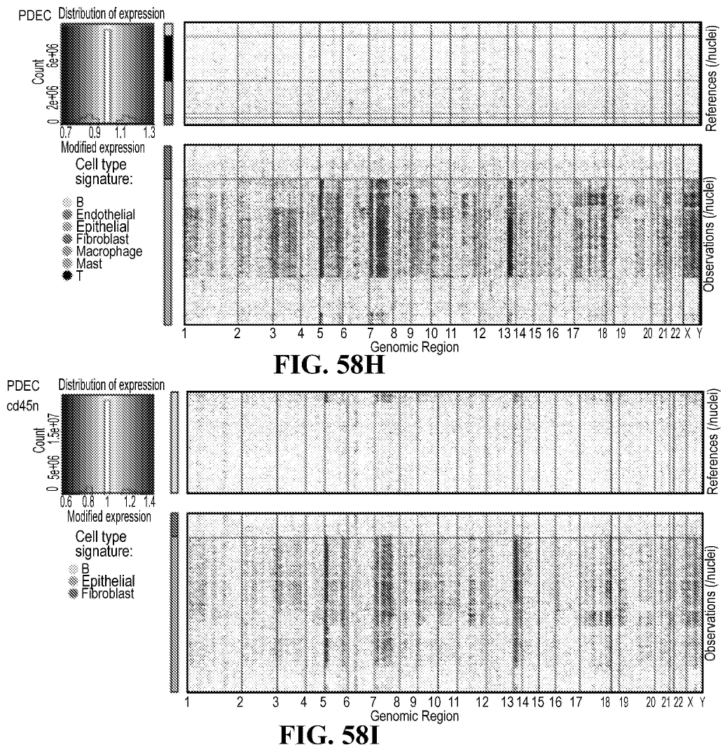

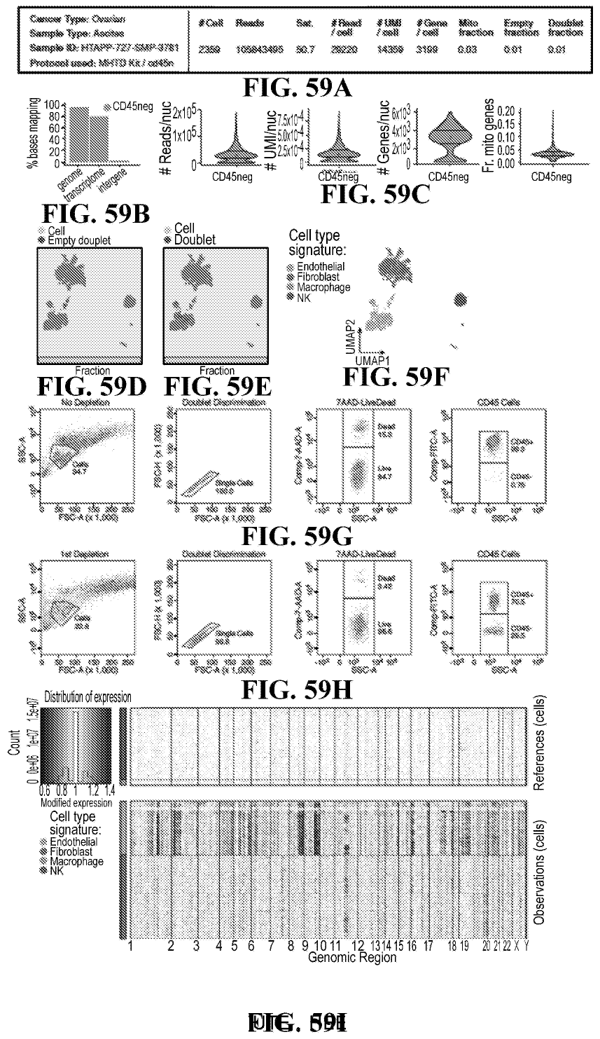

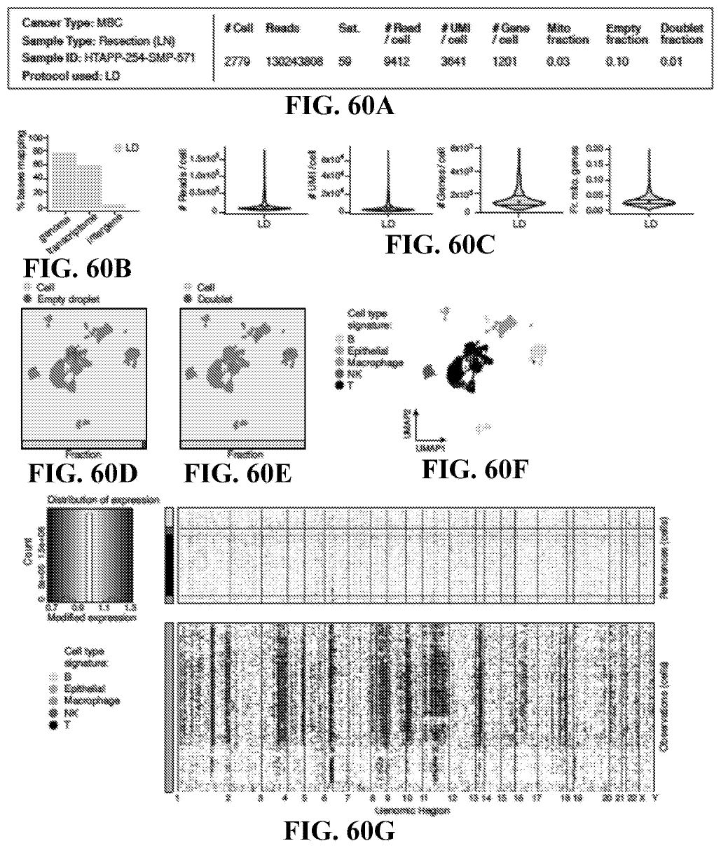

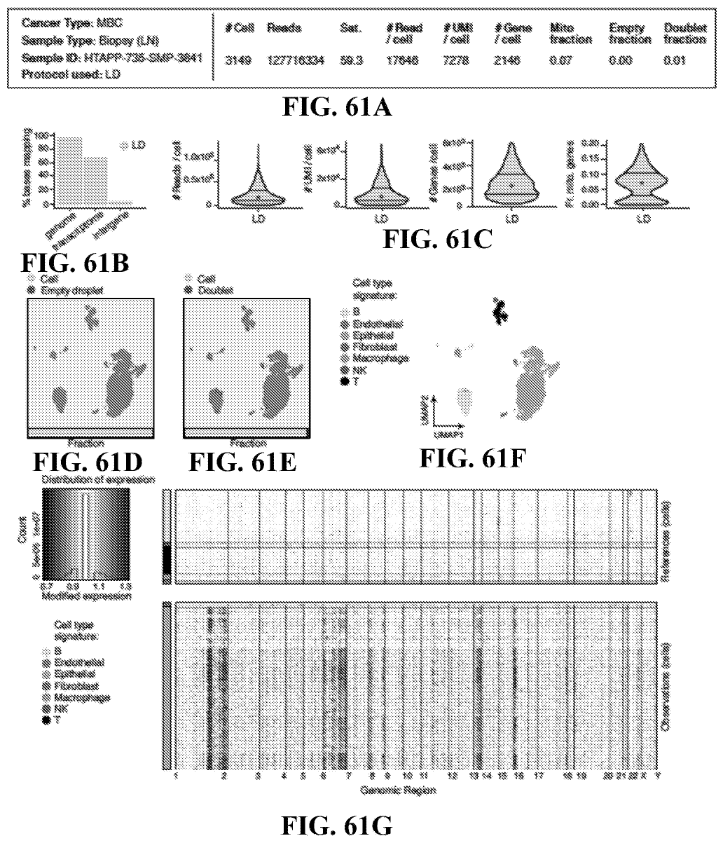

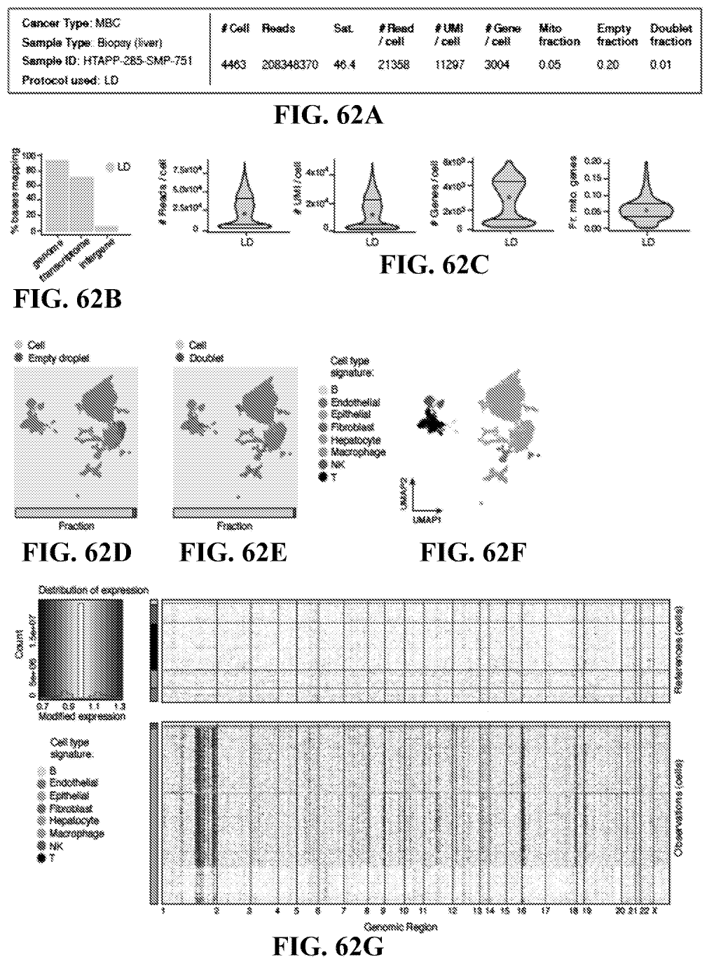

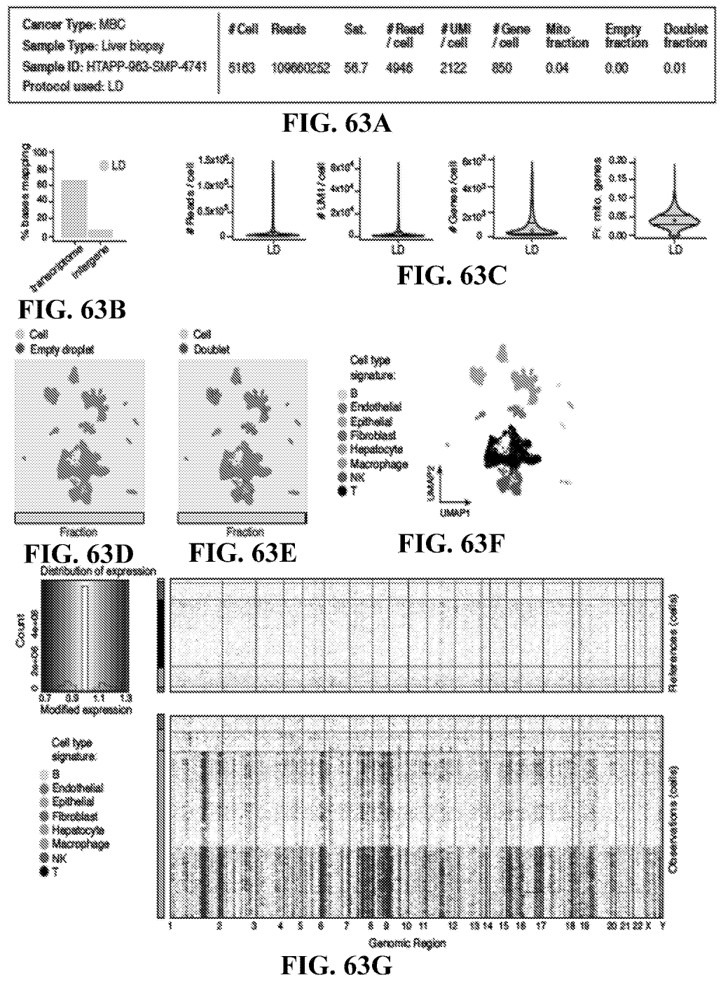

In addition to NSCLC, MBC, ovarian cancer ascites, and neuroblastoma samples (FIG. 53H-53L; FIGS. 56-67), Applicants established effective scRNA-Seq protocols for GBM, ovarian cancer, and CLL (FIGS. 53H-53L; 68-70). In particular, in CLL, Applicants successfully recovered the expected cell types from a cryopreserved sample, containing viable cells. This reflects the increased resilience of immune cells to freezing compared to other cell types, also observed in other settings²¹, and the lack of a dissociation step in CLL scRNA-Seq (Methods). Cryopreservation, however, can increase the proportion of damaged cells²² and may not successfully recover all the malignant and other non-malignant cells in the tumor.

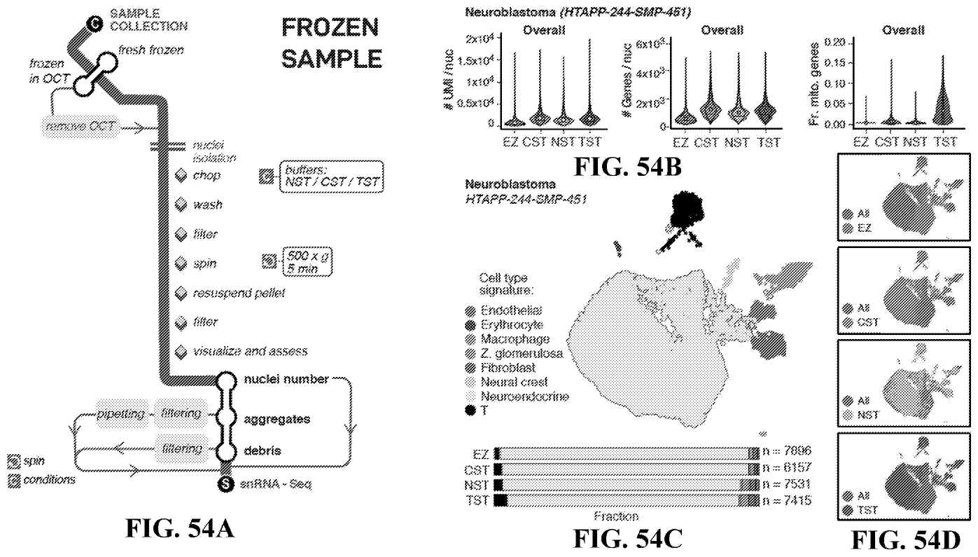

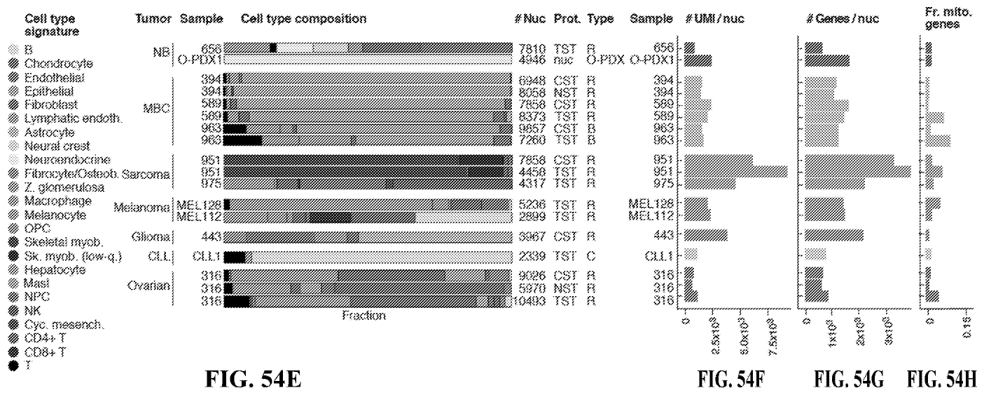

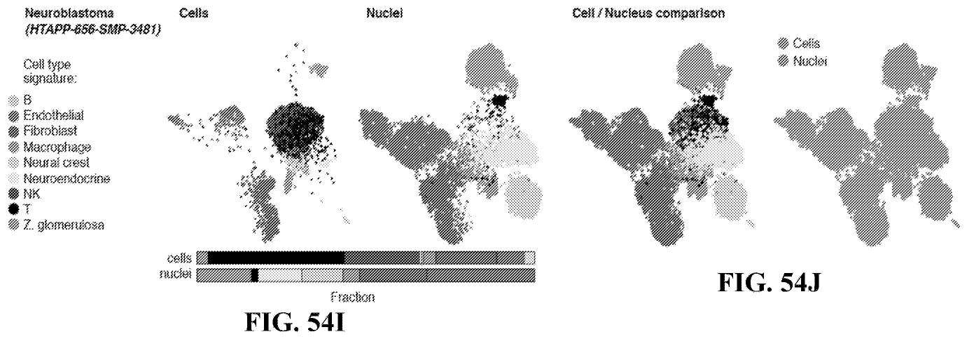

Thus, for frozen specimens from solid tumors, Applicants optimized snRNA-Seq, focusing on different methods for nucleus isolation (FIG. 54A) across seven tumor types: MBC, neuroblastoma, ovarian cancer, pediatric sarcoma, melanoma, pediatric high-grade glioma, and CLL (FIGS. 53B; 55). Applicants initially divided larger samples or used multiple biopsies to compare four isolation methods (EZPrep⁸, Nonidet™ P40 with salts and Tris (NST) [modified from Gao, R., et al²³], CHAPS, with salts and Tris (CST), and Tween with salts and Tris (TST), which differ primarily in the mechanical force (e.g., chopping or douncing), buffer, and/or detergent composition (FIG. 54A, Methods). Because in early tests EZPrep routinely underperformed CST, NST, and TST (data not shown), Applicants only included it in initial comparisons (below). To evaluate protocols, Applicants used the post-hoc computational criteria above, except Applicants excluded the estimation of empty drops, because it was only developed and tested on single-cell RNA-seq data. Applicants further customized scCloud for snRNA-Seq data, mapping reads to both exons and introns, and adapted the QC thresholds for transcript (UMI) and gene counts to reflect the lower expected mRNA content in nuclei (Methods). Experimentally, Applicants added in-process light microscopy QCs to ensure complete nuclei isolation, and to estimate doublets, aggregates, and debris (FIG. 54A, Methods, FIG. 93).

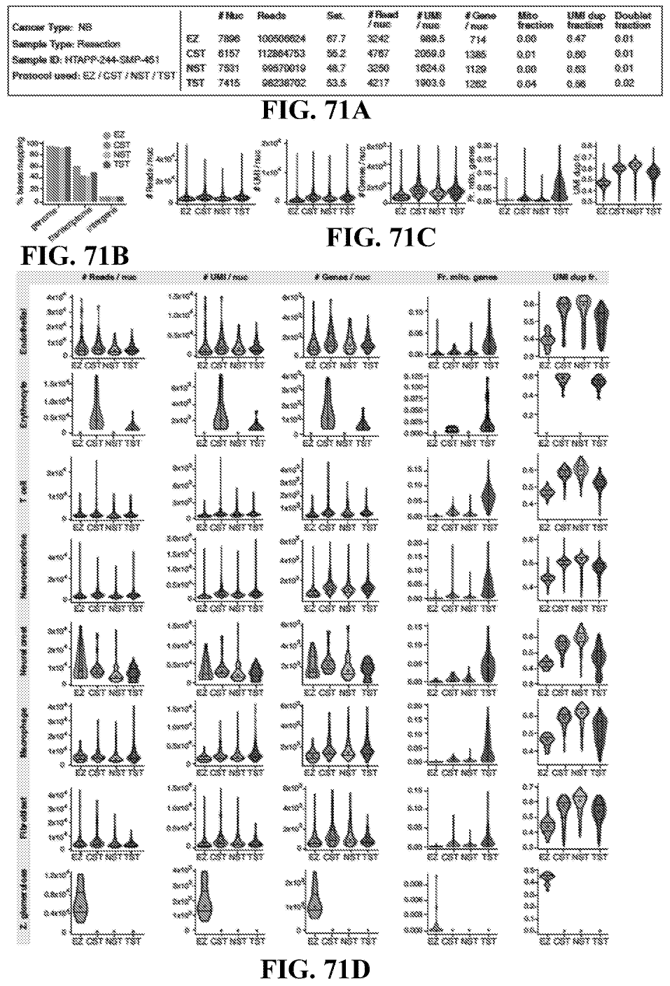

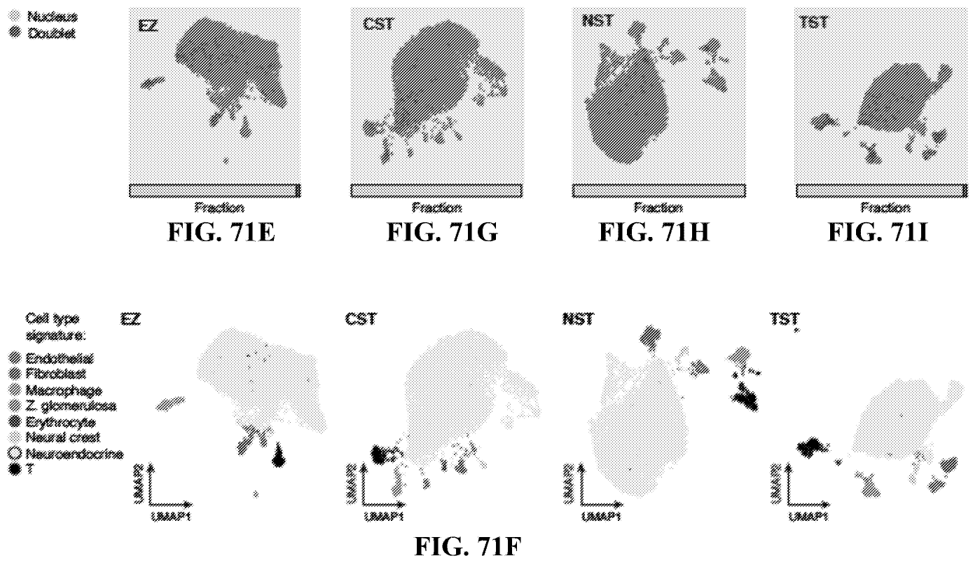

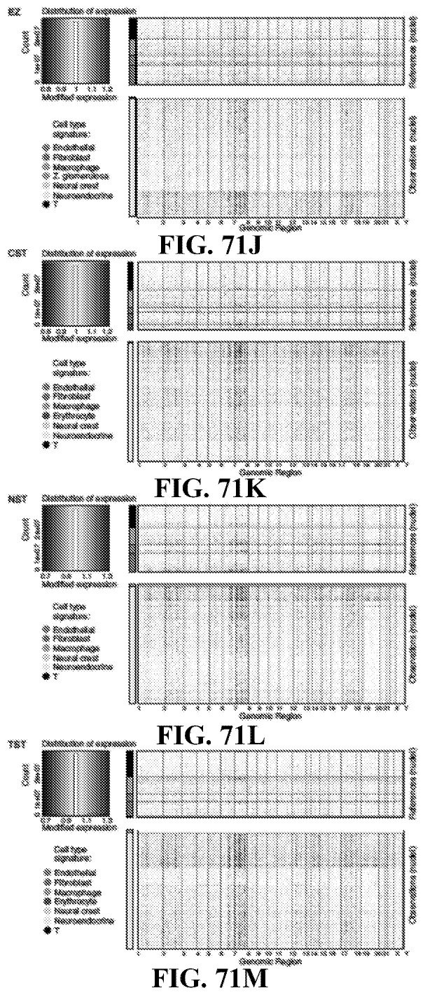

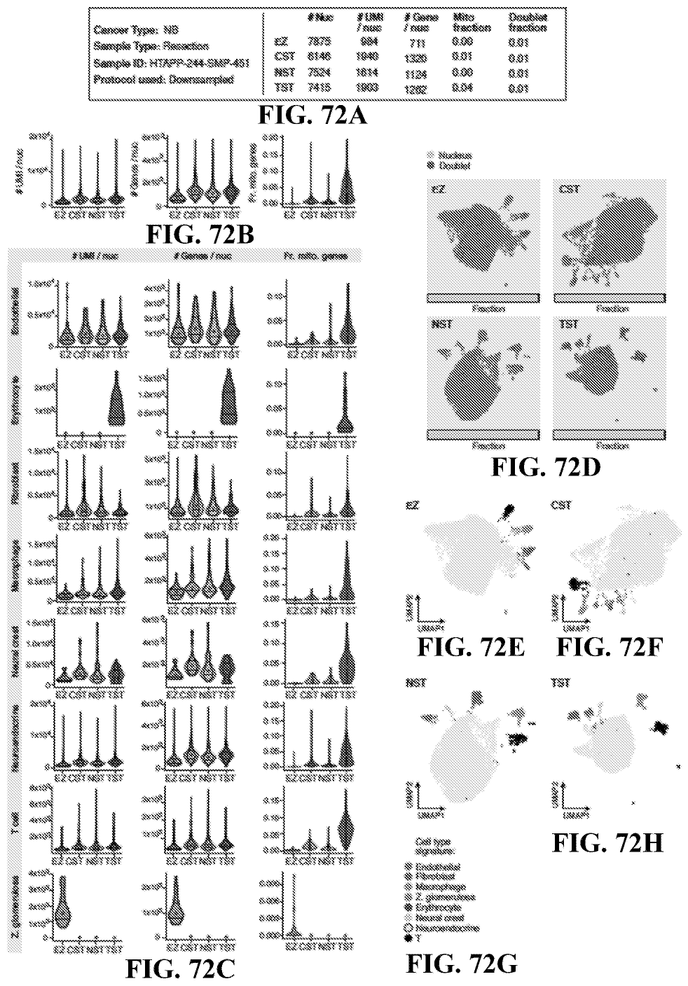

Overall, three nucleus isolation methods—TST, CST, and NST—had comparable performance based on the assessed nucleus quality (FIG. 54B-54H), with TST typically yielding the greatest cell type diversity and number of nuclei per cell type, together with highest expression of mitochondrial genes, and NST typically having the fewest genes per nucleus and lowest diversity of types. For example, in neuroblastoma, testing each of the four protocols on a single resection sample (HTAPP-244) (FIGS. 54B-54D; 71) yielded a similar number of high-quality nuclei (7,896, 6,157, 7,531, and 7,415 for EZ, CST, NST, and TST, respectively), malignant cells with similarly detectable CNAs, and the expected cell types—with malignant neuroendocrine cells being the most prevalent (FIG. 54C; 71D; 71F-71M). However, nuclei prepared with the EZ protocol had lower numbers of UMIs and genes detected (FIG. 54B), while TST recovered more endothelial cells, fibroblasts, neural crest cells, and T cells than the other protocols (FIG. 54C). TST yielded a higher expression of mitochondrial genes (FIG. 54B), in this and all other tumors tested (FIG. 54H), since the nuclear membrane, ER, and ribosomes remain attached to the nucleus when using this method (unpublished results). The same trends were preserved when down-sampling by the total number of sequencing reads (FIG. 72), as well as for cell-type specific QCs (FIG. 71D).

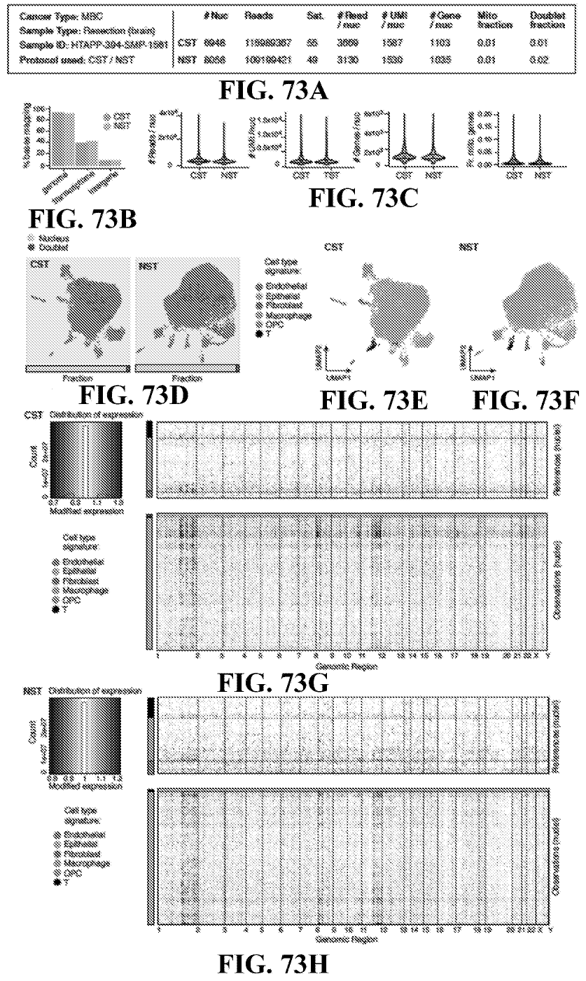

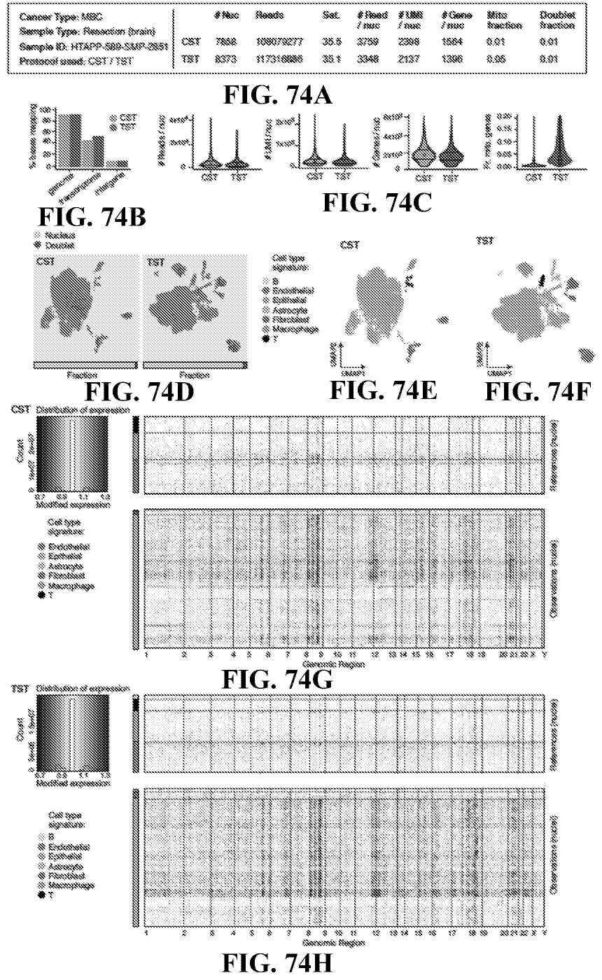

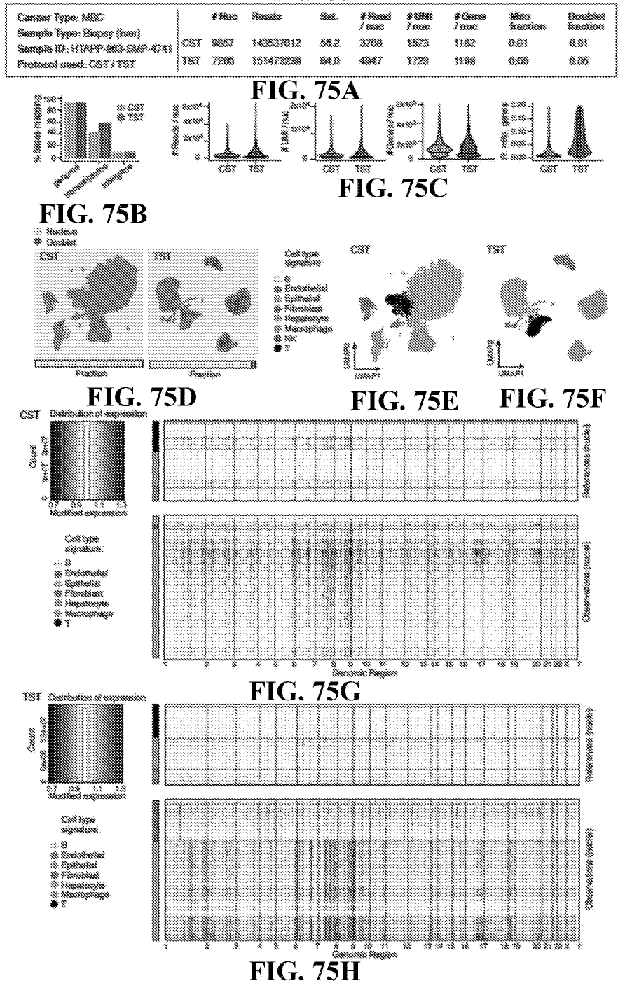

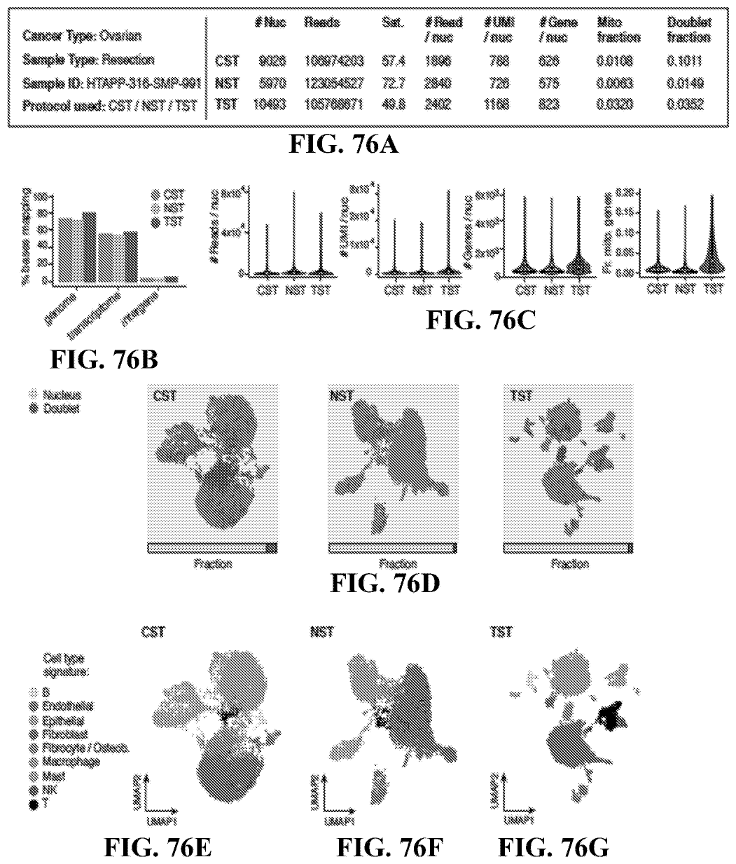

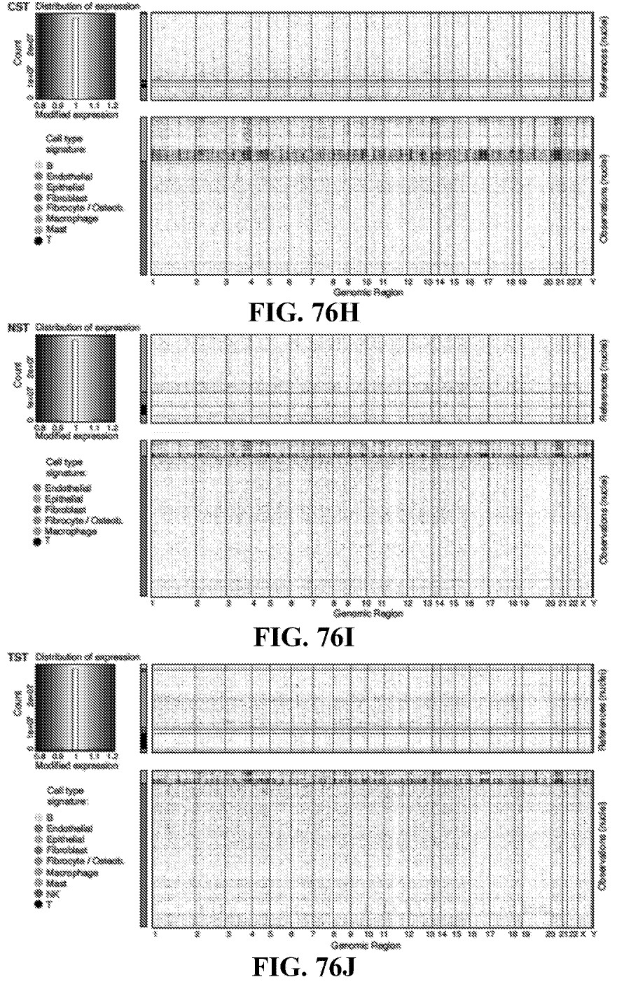

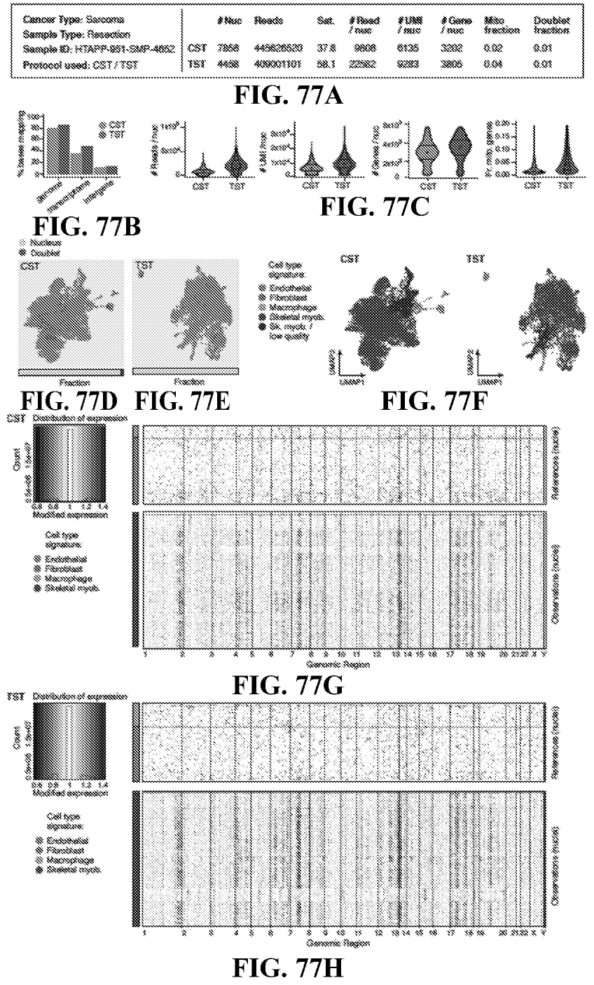

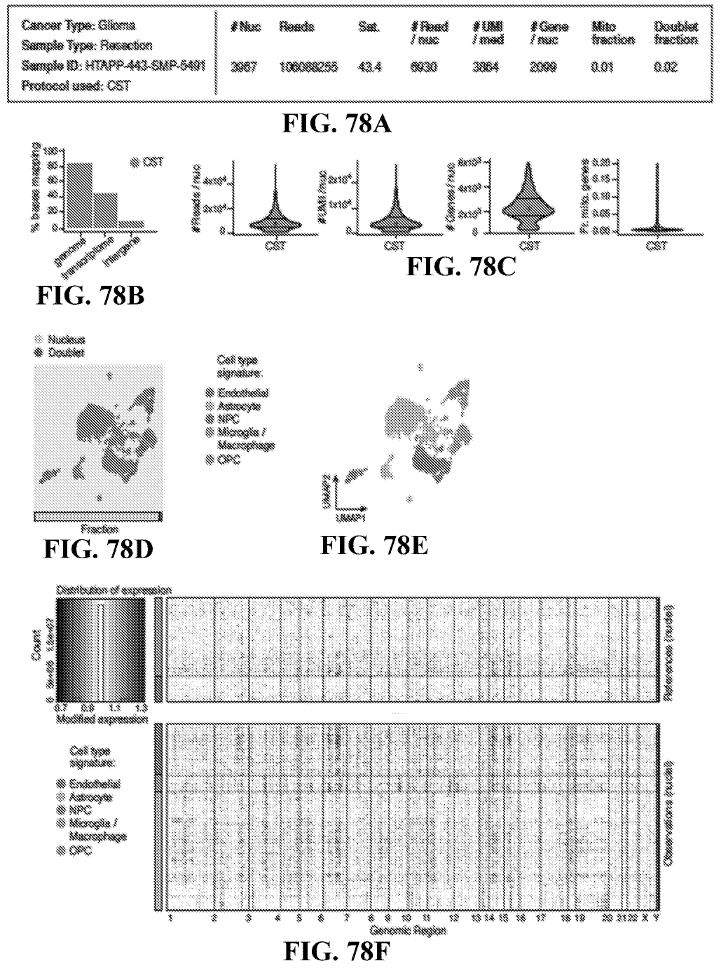

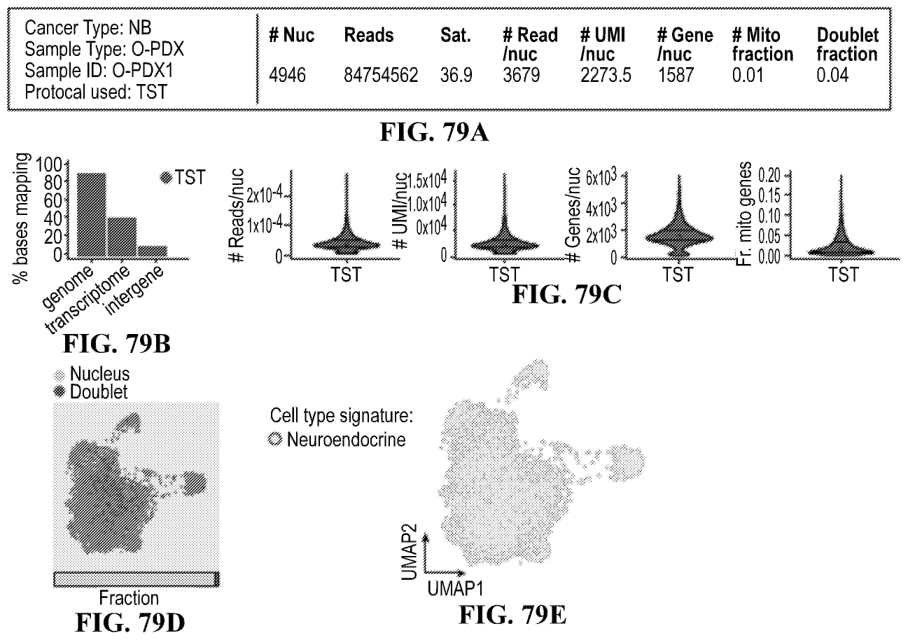

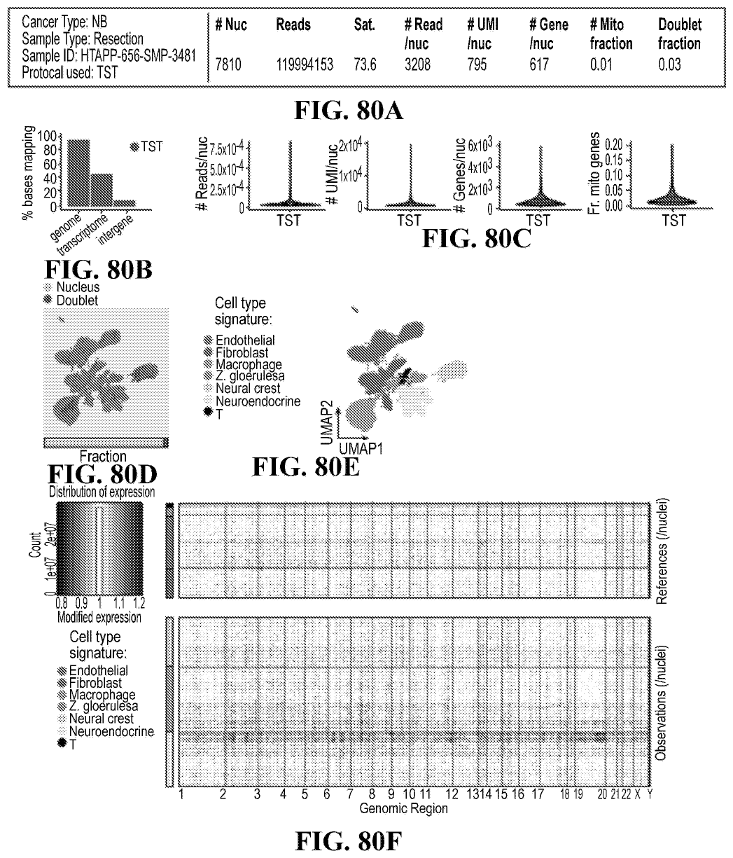

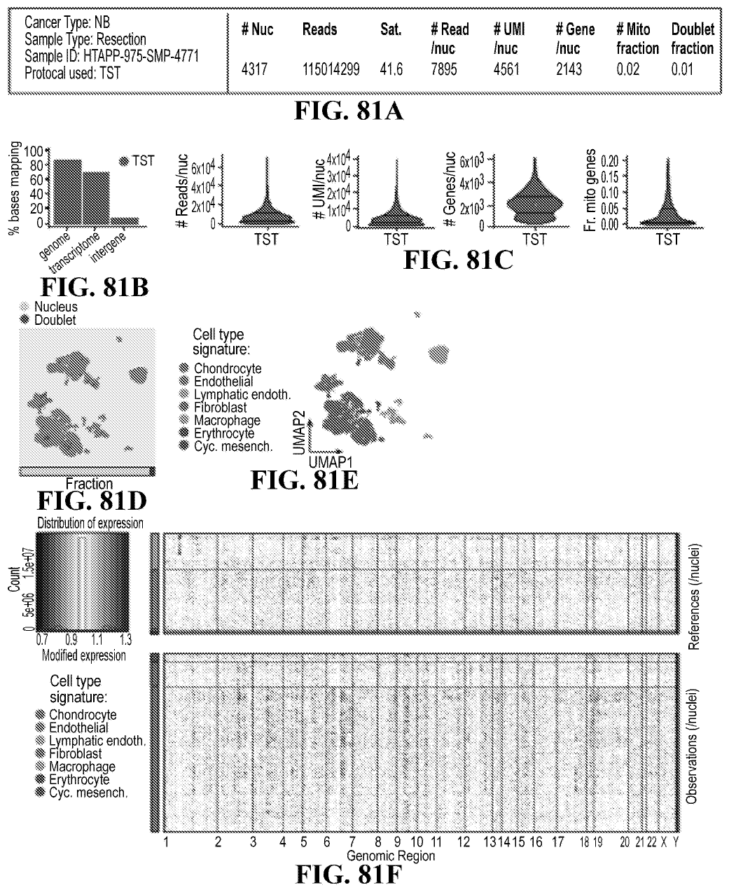

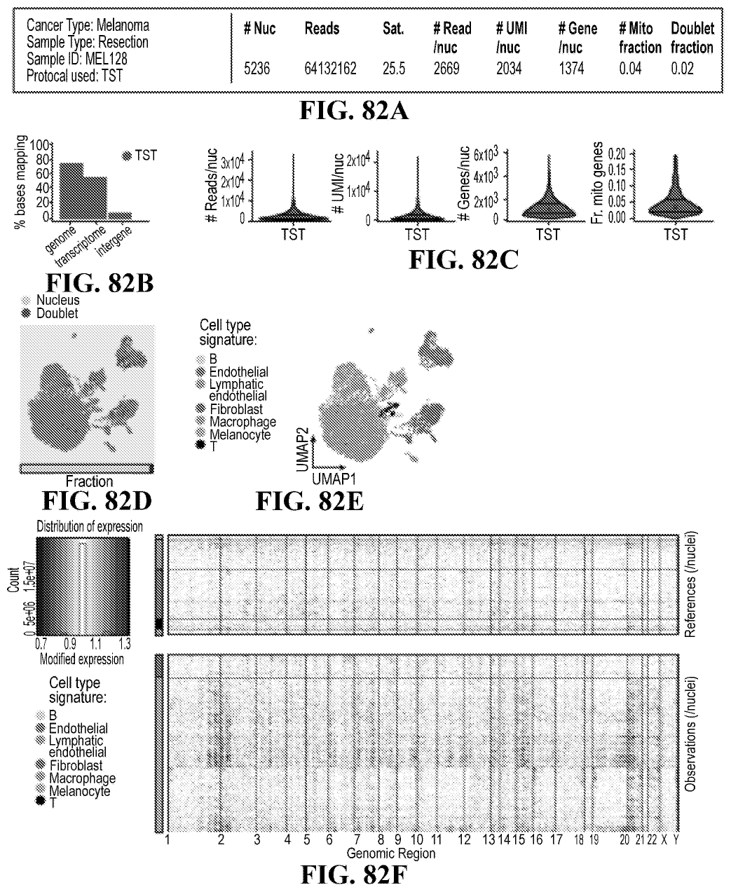

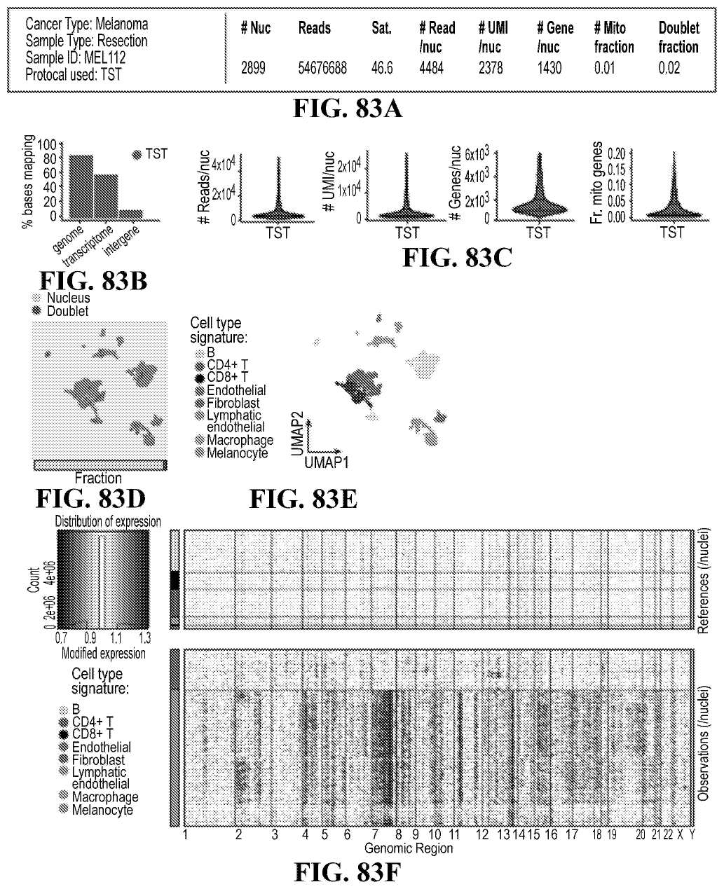

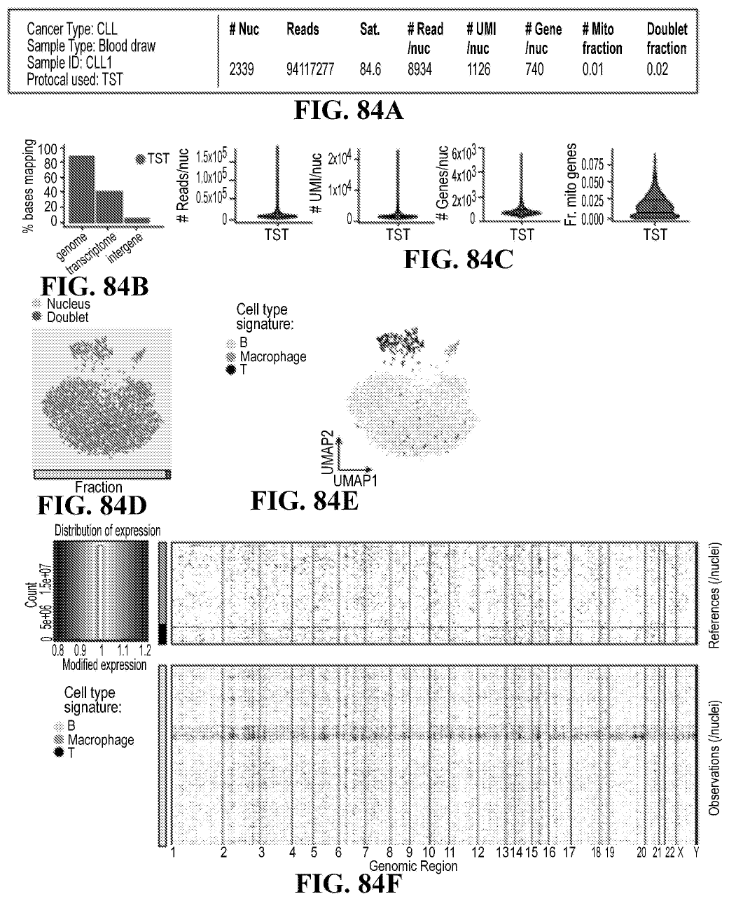

The CST, NST, and TST nucleus isolation methods had similar performance characteristics when tested with MBC, ovarian cancer, and pediatric sarcoma samples, with TST again providing the most diversity in cell types, especially in non-malignant cells. In MBC, Applicants compared CST and NST in one metastatic brain resection (HTAPP-394), and CST and TST in another metastatic brain resection (HTAPP-589) and in a metastatic liver biopsy (HTAPP-963) (FIG. 54E-54H; 73-75). In all cases, QC statistics (FIGS. 54F-54H; 73; 74; 75A-75D) and CNA patterns (FIGS. 73; 74; 75G-75H) were similar between protocols, and nuclei from epithelial cells were the most prevalent (FIG. 54E). CST and NST captured a very similar distribution of cell types, while TST captured more non-malignant cells, including T cells (FIG. 54E) and a higher fraction of mitochondrial reads (FIG. 54H). In ovarian cancer, CST, NST, and TST recovered similar CNA patterns from the same sample (HTAPP-316, FIG. 76), but NST recovered fewer cells, genes per cell, and UMIs per cell (FIG. 54E-54G), and had a lower cell type diversity, despite having greater overall sequencing depth, whereas TST captured the greatest cell type diversity (FIGS. 54E; 76A). In a rhabdomyosarcoma sample (HTAPP-951), CST and TST captured the same cell types at similar proportions (FIG. 54E) and showed similar CNA patterns (FIG. 77).

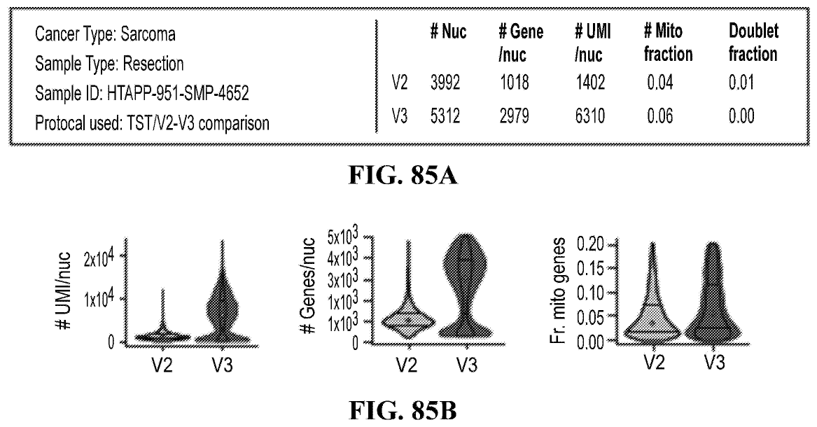

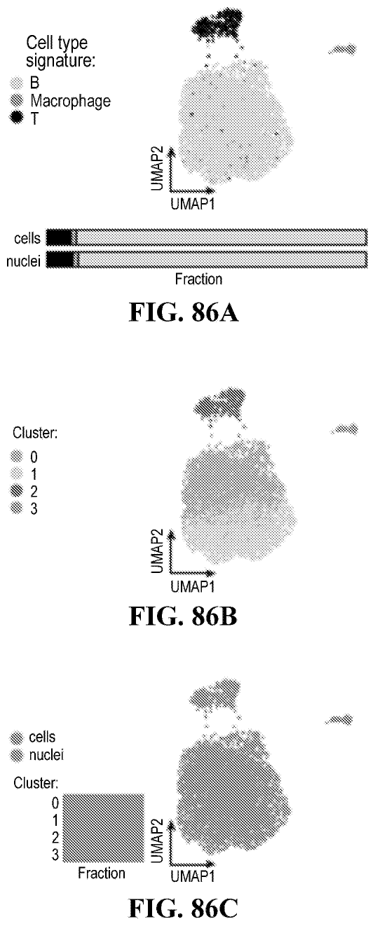

Overall, Applicants recommend the TST protocol for most tumor types, and CST for tumors from neuronal tissues, such as pediatric high-grade glioma (FIGS. 55; 78). With the recommended protocols (FIG. 53B, right column), Applicants profiled additional neuroblastoma tumors as well as Ewing sarcoma, melanoma, pediatric high-grade glioma, and CLL tumor samples—spanning biopsies, resections, and treated samples (FIGS. 53B; 54E-54H; 78-84). Applicants also tested a pediatric rhabdomyosarcoma sample (HTAPP-951) by two different chemistries for droplet based snRNA-Seq (v2 vs. v3 from 10× Genomics, Methods), obtaining overall similar results in terms of cell types detected, an improved number of recovered vs. expected nuclei and higher complexity per nucleus in v3 (FIG. 85).

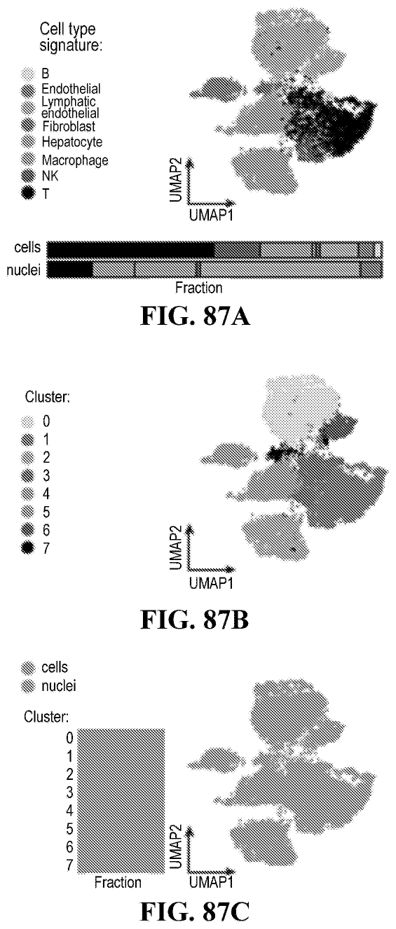

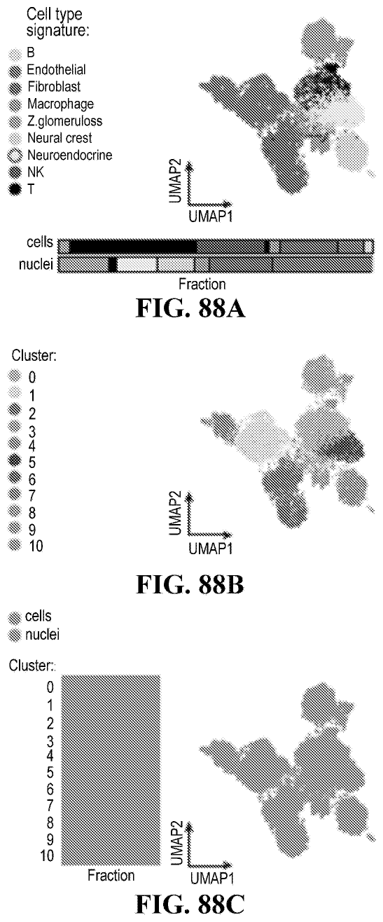

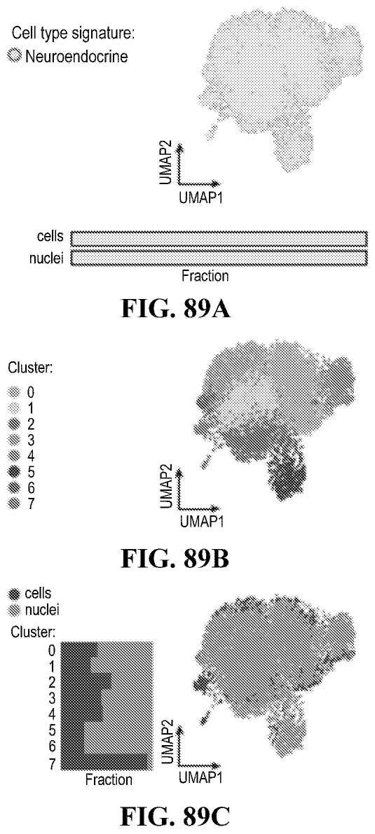

Finally, when Applicants compared scRNA-Seq and snRNA-Seq by testing matching samples from the same specimen each in CLL, MBC, neuroblastoma, and O-PDX (FIGS. 54I-54J; 86-89), the methods typically recovered similar cell types with similar transcriptional profiles, but sometimes at varying proportions. In both neuroblastoma and MBC, immune cells were more prevalent in scRNA-Seq, and parenchymal (especially malignant) cells were more prevalent in snRNA-Seq (FIGS. 87; 88). Cell and nucleus profiles were comparable based on grouping together when using batch correction by canonical correlation analysis (CCA)24 (Methods) (FIGS. 54J; 86-89).

In conclusion, Applicants developed a toolbox for processing fresh and frozen clinical tumor samples by single cell and single nucleus RNA-Seq, and demonstrated it across eight tumor types. For fresh tissues, Applicants recommend testing 2-3 dissociation methods based on the tumor type, the tissue composition and the decision tree (FIG. 53C), and choose to apply the best performing protocol by assessing both experimental and computational QC metrics, and, if desired, adding a depletion step. For frozen tissues, Applicants recommend testing the NST, TST, and CST protocols (FIG. 54A). While TST is often favorable due to its superior ability to capture the most diverse set of cells, in some tumors Applicants recommend CST or NST (e.g., CST for pediatric high-grade glioma, FIG. 55). CST also yields fewer mitochondrial reads, reducing sequencing cost. When possible, Applicants recommend testing both scRNA-Seq and snRNA-Seq for the same tumor type, as the two approaches differ in the distribution of cell types detected. Processing frozen samples by snRNA-Seq allows studying many rare, unusual, and longitudinal banked tumor samples. Our toolbox will help researchers systematically profile additional human tumors, leading to a better understanding of tumor biology and ultimately to an era of precision medicine.

### Example 23

### Experimental Methods for Single-Cell and Single-Nucleus RNA-seq Toolbox for Fresh and Frozen Human Tumors

Human Patient Samples. All work performed for this study was approved by either the Dana-Farber Cancer Institute Institutional Review Board (IRB) [Lung cancer (IRB protocol 98-063), metastatic breast cancer (IRB protocol 05-246), neuroblastoma (IRB protocols 11-104 and 17-104), ovarian cancer (IRB protocol 02-051), melanoma (IRB protocol 11-104), sarcoma (IRB protocol 17-104), GBM(IRB protocol 10-417), and chronic lymphocytic leukemia (IRB protocol 99-224), with secondary use protocol 14-238] or by the St. Jude Children's Research Hospital IRB [pediatric high-grade glioma (IRB protocol 97BANK), neuroblastoma (IRB protocol TBANK [protocol for collecting, banking and distributing human tissue samples: St. Jude Children's Research Hospital Biorepository] for the human samples and MAST [Molecular analysis of solid tumors] for creating O-PDX sample)], and patients were properly consented.

Collection of Fresh Tissue for scRNA-Seq. Collection of fresh solid tumor tissue for lung cancer, ovarian cancer and metastatic breast cancer at BWH/DFCI, was performed following protocols established to reduce the time elapsed between removal of the tumor tissue from the body, placement of the specimen in media, and processing for scRNA-Seq. To this end, Applicants established procedures between the hospital team (surgeon/clinical research coordinator (CRC)/clinical pathologist), the coordinating team (project managers/pathology technician) and the processing team (staff scientists/research technicians) prior to procedure day. This included providing the hospital team with collection containers with appropriate media, and pre-defining allocation priorities to ensure quick handling by the pathology technician of the sample received. On the day of the procedure, timely communication between the teams ensured quick specimen transfer from the hospital team to the research team, timely transport to the Broad for processing, and immediate loading of the single cell suspension into the 10× Genomics Single-Cell Chromium Controller (below).

In all cases, the tissue received from the hospital team was examined by the research pathology technician and following procurement of a specimen for anatomic pathology review, the highest quality portion (or core) was allocated for scRNA-Seq, placed in media and transported to the Broad institute for dissociation following the appropriate protocol (below). Tissue quality is assessed based on visual examination and rapid pathology interpretation at the time of collection, and determined based on tumor content, necrosis, calcification, fat, and hemorrhage.

For ovarian cancer ascites, approximately ˜300 mL were usually received from the hospital team within 1 hour after taken out of the body, which contained a vast majority of non-malignant (mainly immune) cells. Hence, all ascites samples were subjected to CD45+ cell depletion (below) to enrich for malignant cells.

For CLL, samples were generated from peripheral blood mononuclear cells isolated using density centrifugation (Ficoll-Paque) and stored in freezing media (FBS+10% DMSO) in liquid nitrogen until processing.

For orthotopic PDX of neuroblastoma samples (O-PDX), Foxn1−/− nude mice (Charles River Laboratories) were orthotopically injected via ultrasound-guided para-adrenal injection with cells derived from a patient MYCN-amplified neuroblastoma (available as sample SJNBL046_X1 through the Childhood Solid Tumor Network^{19,20}. A portion of O-PDX tumor was flash-frozen for future single-nucleus RNA-Seq, while the remainder underwent dissociation as described below.

Preservation of Tissue for snRNA-Seq. For those samples that were prospectively collected by Applicants for snRNA-seq (Neuroblastoma HTAP), freezing of tumor samples was performed as quickly as possible after sample collection using standard biobanking technique and the dates when samples were frozen were recorded. (Other samples were obtained from tissue banks with limited record on how they were frozen, which is a typical scenario.) Samples were placed in cryo-tubes without any liquid. Complete removal of liquid from the sample was accomplished by gently wiping it (not patting, as this would damage the tissue) on the side of the container, before placing in the cryotube. The tubes were then covered in dry-ice and transferred to −80° C. for long term storage.

The other frozen samples from snRNA-Seq were obtained from tissue banks as follows: Ovarian OCT-frozen archival samples were obtained from the Dana-Farber Cancer Institute Gynecology Oncology Tissue Bank; sarcoma snap-frozen samples were obtained from the Boston Children's Hospital Tissue Bank; pediatric snap-frozen glioma samples were obtained from the St. Jude Children's Research Hospital Biorepository; neuroblastoma snap-frozen samples were obtained from the St. Jude Children's Research Hospital Biorepository and the Boston Children's Hospital Precision Link Biobank for Health Discovery; metastatic breast cancer OCT-frozen samples were obtained from the Center for Cancer Precision Medicine Bank; snap-frozen melanoma samples were obtained through the laboratory of Dr. Charles Yoon at BWH.

### Example 24

### Dissociation Workflow from Fresh Solid Tumor Samples

MBC, NSCLC (protocols PDEC and LE), ovarian cancer solid tumor, and neuroblastoma workflows. Fresh tissue dissociation of MBC, NSCLC (protocols PDEC and LE), ovarian cancer solid tumor, and neuroblastoma were performed using a similar workflow (FIG. 53C), with different components of the dissociation mixture for each tumor type, as described in the next section.

Samples were transferred from interventional radiology (biopsies) or the operating room (resections) in DMEM (MBC), RPMI (NSCLC), or RPMI with HEPES (ovarian cancer and neuroblastoma) medium. Upon arrival to the laboratory, the sample was washed in cold PBS and transferred into either a 2 mL Eppendorf tube containing dissociation mixture (for biopsies) or a 5 mL Eppendorf tube containing 3 mL dissociation mixture (for resections). Next, the sample was minced in the Eppendorf tube using spring scissors (Fine Science Tools, catalog no. 15514-12) into fragments under ˜0.4 mm, and incubated at 37° C., while rotating at approximately 14 RPM, for 10 minutes. After 10 minutes, the sample was pipetted 20 times with a 1 mL pipette tip at room temperature, and placed back into incubation with rotation for an additional 10 minutes. The sample was pipetted again 20 times using a 1 mL pipette tip, and transferred to 1.7 mL Eppendorf tube and centrifuged at 300 g for 4 minutes at 4° C. The supernatant was removed and the pellet was resuspended in 200-500 μL of ACK red blood cell lysis buffer (Thermo Fisher Scientific, A1049201). The ACK volume added depended on the size of the pellet; while pellet size is hard to quantify Applicants suggest adding about 100 μL ACK lysis buffer per 100,000 cells, with a minimum volume of 200 μL. The sample was incubated in ACK red blood cell lysis buffer for 1 minute on ice, followed by the addition of cold PBS at twice the volume of the ACK. The cells were pelleted by a short centrifugation for 8 seconds at 4° C. using the short spin setting with centrifugal force ramping up to, but not exceeding, 11,000 g. The supernatant was removed. The pellet color was assessed, if RBCs remained (pellet color pink or red), the ACK step was repeated up to two additional times. To remove cell clumps, the pellet was resuspended in 100 μL of TrypLE (Life Technologies, catalog no. 12604013) and incubated while constantly pipetting at room temperature for 1 minute with a 200 μL pipette tip. TrypLE was inactivated by adding 200 μL of cold RPMI 1640 with 10% FBS. The cells were pelleted using short centrifugation as described above. The pellet was resuspended in 50 μL of 0.4% BSA (Ambion, catalog no. AM2616) in PBS. To assess the single cell suspension, viability, and cell count, 5 μL of Trypan blue (Thermo Fisher Scientific, catalog no. T10282) was mixed with 5 μL of the sample and loaded on INCYTO C-Chip Disposable Hemocytometer, Neubauer Improved (VWR, catalog no. 82030-468). The cell concentration was adjusted if necessary to a range of 200-2,000 cells/μL. A total of 8,000 cells were loaded into each channel of the 10× Genomics Single-Cell Chromium Controller. Due to differences between clinical samples, some steps may need to be repeated or adjusted; for a general overview of guidelines see FIG. 53C.

NSCLC-C4 protocol workflow. A similar workflow was used for protocol NSCLC-C4 with the following modifications: Following mechanical chopping as above, sample was dissociated for 15 minutes in a 15 mL falcon tube, with gentle vortex every 5 minutes, followed by filtration through a 70 μm filter, and washed with 20 mL of ice cold PBS and centrifuged at 580 g for 5 minutes. RBS lysis was performed similarly to the above workflow by resuspending the pellet in 1 mL ACK lysis buffer with incubation on ice for 1 minute. 20 mL of ice cold PBS were added to quench the ACK lysis buffer, followed by filtration through a 70 μm filter, and centrifugation at 580 g for 5 minutes. The sample was then cleaned using Viahance™ dead-cell removal kit (BioPAL, catalog no. CP-50VQ02) according to manufacturer's instructions. Cells were then re-suspended in M199 and loaded on the 10× Genomics Single-Cell Chromium Controller as described above.

GBM workflow. All steps were done on ice. Sample was minced thoroughly in Petri dish, thereafter, 4 mL HBSS were added (Life Technologies, catalog number 14175095), transferred to 15 mL tubes and centrifuged at 1000 rpm for 2 minutes. After centrifugation, supernatant was removed, pre-heated Buffer X was added, and the sample was incubated while shaking at 37° C. for 15 minutes. Sample was pipetted up-down 20 times, incubated at 37° C. for an additional 15 minutes, and pipetted again. After dissociation, the sample was filtered through a 100 μm cell strainer (Fisher Scientific, Cat #22-363-547) into 50 mL tube. Applicants recommend keeping any tissue fragments left in the cell strainer, as they can be reprocessed with the same protocol if initial cell recovery is low. Filtrate was centrifuged at 1000 rpm for 3 minutes, and the supernatant was removed. If the pellet was bloody, RBC removal was performed when needed using LYMPHOLYTE H (CedarLAne, Cat. #CL5015) or Red Blood Cell (RBC) Lysis Solution (10×) (Miltenyi Biotech, Cat #130-094-183). The pellet was washed with 10 mL of cold PBS/1% BSA, transferred to 15 mL tube and centrifuged at 1200 rpm for 3 minutes. Supernatant was removed and the pellet was resuspended in 0.4 BSA in PBS. Single cell suspension was visualized, counted and loaded on the 10× Genomics Single-Cell Chromium Controller as described above.

Dissociation mixtures for different tumor types. Dissociation mixtures were prepared approximately 5-10 minutes before sample processing from frozen aliquoted stocks, as follows:

MBC, LD Protocol. 950 μl of RPMI 1640 (Thermo Fisher Scientific, catalog no. 11875093), 10 μL of 10 mg/mL DNAse I (Sigma Aldrich, catalog no. 11284932001) to a final concentration of 100 μg/mL, and 40 μL of 2.5 mg/mL Liberase™.

Ovarian cancer resection. Dissociation mixture was based on Miltenyi Human Tumor Dissociation Kit (Miltenyi Biotec, catalog no. 130-095-929). Before starting, Enzymes H, R, and A were resuspended according to manufacturer's instructions. Dissociation mix containing 2.2 mL RPMI, 100 μL enzyme H, 50 μL enzyme R, and 12.5 enzyme A, was prepared immediately before use.

Neuroblastoma, NB-C4 protocol. Medium 199, Hanks Balanced Salts Buffer (Thermo Fisher Scientific) with 100 μg/mL of DNAse I (Millipore Sigma, catalog no. 11284932001), 100 μg/mL Collagenase IV (Worthington; catalog no. LS004186).

Orthotopic PDX neuroblastoma. Worthington Papain Dissociation System (catalog no. LK003150). Dissociation was performed according to manufacturer's instructions, with deviation of the dissociation duration, which was shortened to 15 minutes.

NSCLC, PDEC protocol. 2692 HBSS (Thermo Fisher Scientific, catalog no. 14170112), 187.5 μL of 20 mg/mL pronase (Sigma Aldrich, catalog no. 10165921001) to a final concentration of 1,250 μg/mL, 27.6 μL of 1 mg/mL elastase (Thermo Fisher Scientific, catalog no. NC9301601) to a final concentration of 9.2 μg/mL, 30 μL of 10 mg/mL DNase I (Sigma Aldrich, catalog no. 11284932001) to a final concentration of 100 μg/mL, 30 μL of 10 mg/mL Dispase (Sigma Aldrich, catalog no. 4942078001) to a final concentration of 100 μg/mL, 30 μL of 150 mg/mL Collagenase A (Sigma Aldrich, catalog no. 10103578001) to a final concentration of 1,500 μg/mL, 3 μL of 100 μg/mL collagenase IV (Thermo Fisher Scientific, catalog no. NC9836075) to a final concentration of 1250 μg/mL.

NSCLC, LE protocol. 5 mL RPMI 1640 (Thermo Fisher Scientific, catalog no. 11875093), 200 μL of 2.5 mg/mL Liberase™ (Millipore Sigma, 5401119001) to a final concentration of 100 μg/mL, 50 μL of 10 mg/mL DNase I (Sigma Aldrich, catalog no. 11284932001) to a final concentration of 100 μg/mL, 27.6 μL of 1 mg/mL elastase (Thermo Fisher Scientific, catalog number NC9301601) to a final concentration of 9.2 μg/mL.

NSCLC, C4 protocol. 5 mL M199 with DNase 1 (final concentration of 10 μg/mL) and Collagenase iv (final concentration of 100 μg/mL).

GBM. Brain Tumor Dissociation Kit (P) (Miltenyi Biotech. Catalog number 130-095-942). 4 mL Buffer X, 40 μL Buffer Y, 50μ Enzyme N, 20μ Enzyme A.

Processing of Non-Solid Tumor Samples for scRNA-Seq

CLL. Frozen (cryopreserved) cells were thawed in 10 mL RPMI, pelleted and washed with an additional 10 mL RPMI. Live cells were sorted using the MoFlo Astrios EQ Cell Sorter, and 8,000 cells were loaded on one channel of the 10× Genomics Single-Cell Chromium Controller. Remaining cells were pelleted by short centrifugation, the supernatant was discarded and the pellet was frozen on dry ice and stored in −80° C.

Ovarian cancer ascites. Ascites samples without spheres were selected and delivered in six 50 mL conical tubes, for a total of 300 mL of fluid. Tubes were spun down at 580×g for 5 minutes in a 4° C. pre-cooled centrifuge and supernatants was aspirated.

Pellets were resuspended in 5 mL cold ACK Lysing Buffer, and combined from all tubes at this step. ACK lysis was done on ice for 3 minutes, and quenched by adding 10 mL of cold PBS, followed by centrifugation at 580×g for 5 minutes at 4° C. The pellet color was assessed and if it was pink or red, revealing a significant portion of erythrocytes, ACK treatment steps were repeated as needed at most two additional times. Post ACK treatment, the pellet was resuspended in 20 mL cold PBS, filtered through a 70 μm cell strainer into a 50 mL conical tube, and the filter was washed with additional 20 mL cold PBS to recover as many cells as possible. The sample was then centrifuged at 580×g for 5 minutes at 4° C. To reduce the fraction of immune cells in the sample, CD45+ cell depletion was performed using the MACS CD45 depletion protocol described below.

Depletion of CD45+ cells for scRNA-Seq. Depletion of CD45+ cells in ovarian cancer ascites samples and NSCLC was performed using CD45 MicroBeads (Miltenyi Biotec, catalog no. 130-045-801) according to the manufacturer's protocol. Briefly, following dissociation of ascites or NSCLC samples, cells were counted. The single-cell suspension was centrifuged at 500 g for 4 minutes at 4° C. The supernatant was removed and the pellet was resuspended in 80 μL of MACS buffer (PBS supplemented with 0.5% BSA, and 2 mM EDTA) per 106 cells. 20 μL of the MACS CD45 microbeads were added to the cell suspension per 10 million cells. The cells incubated on ice for 15 minutes. During the incubation, the LS column was prepared by attaching the column to a MidiMACS separator and rinsing the column with 3 mL MACS buffer. Following the incubation, the cells and bead conjugate was washed with 900 μL MACS buffer per 10 million cells. The cells were centrifuged at 500 g for 4 minutes at 4° C. The supernatant was removed and the pellet was resuspended in 500 μL MACS buffer. The cell suspension was transferred to the LS column and the effluent was collected (CD45− fraction). The column was washed three times with 3 mL MACS buffer. The CD45− fraction was centrifuged at 500 g for 4 minutes at 4° C. In ascites samples, bead attachment and column separation can be repeated to increase the number of tumor and stromal cells relative to immune cells. The pellet was resuspended in 50 μL of 0.4% BSA (Ambion, catalog no. AM2616) in PBS. Cells were counted by mixing 5 μL of Trypan blue (Thermo Fisher Scientific, catalog no. T10282) with 5 μL of the sample and loaded on INCYTO C-Chip Disposable Hemocytometer, Neubauer Improved (VWR, catalog no. 82030-468). The cell concentration was adjusted if necessary to a range of 200-2,000 cells/μL. 8,000 cells were loaded into each channel of the 10× Genomics Single-Cell Chromium Controller.

ST based buffers for snRNA-seq. 2× stock of salt-Tris solution (ST buffer) containing 146 mM NaCl (Thermo Fisher Scientific, catalog no. AM9759), 10 mM Tris-HCl pH 7.5 (Thermo Fisher Scientific, catalog no. 15567027), 1 mM CaCl2 (Vwr, catalog no. 97062-820), and 21 mM MgCl2 (Sigma-Aldrich, catalog no. M1028) was made and used to prepare three buffers. For CST: 1 mL of 2× ST buffer, 980 μL of 1% CHAPS (Millipore), 10 μl of 2% BSA (New England BioLabs), and 10 μL of nuclease-free water. For TST: 1 mL of 2× ST buffer, 60 μL of 1% Tween-20 (Sigma-aldrich, catalog no. P-7949), 10 μL of 2% BSA (New England Biolabs, catalog no. B9000S), and 930 μL of nuclease-free water. For NST: 1 mL of 2× ST buffer, 40 μL of 10% Nonidet™ P40 Substitute (Fisher Scientific), 10 μL of 2% BSA (NEB), and 950 μL of nuclease-free water. 1× ST buffer was prepared by dilution 2× ST with ultra-pure water (Thermo Fisher Scientific catalog no. 10977023) in a ratio of 1:1.

Nucleus isolation from frozen samples for snRNA-seq. On dry ice, tissue was split and subjected to one of three salt-Tris (ST)-based nucleus isolation protocols (ED, NVW, CS, ORR and AR; unpublished results) and the EZ nuclei isolation buffer8, as detailed below.

Nucleus isolation workflow for ST-based buffers. On ice, a piece of frozen tumor tissue was placed into a well of a 6-well plate (Stem cell Technologies, catalog no. 38015) with 1 mL of either CST, TST, or NST buffer. For samples frozen in OCT, an additional step of removing the surrounding OCT, and washing any residual OCT from the sample with PBS was performed in a 10 cm Petri dish. Tissue was then chopped using Noyes Spring Scissors (Fine Science Tools, catalog no. 15514-12) for 10 minutes on ice. For cell pellets, such as for CLL frozen cells, sample was pipetted in the buffer on ice, instead of chopping. The homogenized solution was then filtered through a 40 μm Falcon™ cell strainer (Thermo Fisher Scientific, catalog no. 08-771-2). An additional 1 mL of the detergent buffer solution was used to wash the well and filter. The volume was brought up to 5 mL with 3 mL of 1× ST buffer. The sample was then transferred to a 15 mL conical tube and centrifuged at 4° C. for 5 minutes at 500 g in a swinging bucket centrifuge. The pellet was resuspended in 1× ST buffer. Resuspension volume was dependent on the size of the pellet, usually within the range of 100-200 μL. The nucleus solution was then filtered through a 35 μm Falcon™ cell strainer (Corning, catalog no. 352235). Nuclei were counted using C-chip disposable hemocytometer (VWR International Ltd, catalog no. 22-600-100). 10,000 or 8,000 nuclei (V2 or V3 10× genomics, receptively) of the single-nucleus suspension were loaded onto the Chromium Chips for the Chromium Single Cell 3′ Library (V2, PN-120233; V3 PN-1000075) according to the manufacturer's recommendations (10× Genomics).

Nucleus isolation workflow using EZ lysis buffer. Nucleus isolation was done as previously described8. Briefly, tissue samples were cut into pieces <0.5 cm and homogenized using a glass dounce tissue grinder (Sigma, Catalog no. D8938). The tissue was homogenized 25 times with pestle A and 25 times with pestle B in 2 mL of ice-cold nuclei EZ lysis buffer. The sample was then incubated on ice for 5 minutes, with an additional 3 mL of cold EZ lysis buffer. Nuclei were centrifuged at 500 g for 5 minutes at 4° C., washed with 5 mL ice-cold EZ lysis buffer and incubated on ice for 5 minutes. After centrifugation, the nucleus pellet was washed with 5 mL Nuclei Suspension Buffer (NSB; consisting of lx PBS, 0.01% BSA and 0.1% RNAse inhibitor (Clontech, Catalog no.2313A)). Isolated nuclei were resuspended in 2 mL NSB, filtered through a 35 μm cell strainer (Corning-Falcon, Catalog no. 352235) and counted. A final concentration of 1,000 nuclei/μL was used for loading on 10× v2 channel.

Droplet-based sc/snRNA-seq. An input of 8,000 single cells or 10,000 single nuclei (8,000 for v3 10× technology) were loaded into each channel of the Chromium single cell 3′ Chip. Single cells/nuclei were partitioned into droplets with Gel Beads in the Chromium. After emulsions were formed, barcoded reverse transcription of RNA took place. This was followed by cDNA amplification, fragmentation and adaptor and sample index attachment, all according to the manufacturer's recommendations. Libraries from four 10× channels were pooled together and sequenced on one lane of an Illumina HiSeqX with paired end reads, Read 1: 26 nt, Read 2: 55 nt, Index 1: 8 nt, Index 2: 0 nt.

Computational Methods

scRNA-seq data processing. Applicants used Cell Ranger mkfastq (v2.0 and v3.0) (10× Genomics) to generate demultiplexed FASTQ files from the raw sequencing reads. Applicants aligned these reads to the human GRCh38 genome and quantified gene counts as UMIs using Cell Ranger count (v2.0 and v3.0) (10× Genomics). For single-nucleus RNA-seq reads, Applicants counted reads mapping to introns as well as exons, as this results in a greater number of genes detected per nucleus, more nuclei passing quality control, and better cell type identification, as previously described²⁵. To count introns during read mapping, Applicants followed the approach described at https://support.10×genomics.com/single-cell-gene-expression/software/pipelines/latest/advanced/references. Briefly, Applicants built a “pre-mRNA” human GRCh38 reference using Cell Ranger mkref (v3.0) (10× Genomics) and a modified gene transfer format (GTF) file, where for each transcript, the feature type had been changed from transcript to exon. The starting GTF files came from refdata-cellranger-GRCh38-1.2.0.tar.gz or refdata-cellranger-GRCh38-3.0.0.tar.gz, and are available for download at https://support.10×genomics.com/single-cell-gene-expression/software/downloads/3.0.

To down-sample sequencing reads or gene counts (UMIs) when comparing protocols, Applicants used downsampleReads and downsampleMatrix, respectively from the R package¹⁰ DropletUtils. Reads were down-sampled to match the protocol with the lowest number of total reads. After down-sampling by total reads, Applicants used write10×Counts from DropletUtils and a custom python script to generate an HDF5 file for input into the analysis pipelines described below.

Quality control of scRNA-seq data. To maintain explicit control over all gene and cell quality control filters, in all the downstream analyses Applicants used the raw feature-barcode matrix, rather than the filtered feature-barcode matrix generated by Cell Ranger. Applicants removed low quality cells by requiring each cell to have a minimal number of UMIs and genes detected. Applicants used different thresholds depending on the experimental modality (single cell or single nucleus) and on the 10× kit (V2 or V3 chemistry). For single nucleus data, Applicants retained nuclei with at least 200 genes and 400 UMIs detected by V2 chemistry and with at least 500 genes and 1,000 UMIs detected by V3 chemistry. For single cell data, Applicants retained cells with at least 500 genes and 1,000 UMIs detected by either V2 or V3 chemistry. For both data types, Applicants filtered out those cells or nuclei where >20% of UMIs came from mitochondrial genes. Finally, Applicants normalized the total UMIs per cell or nucleus to one-hundred thousand (CP100K), and log-transformed these values to report gene expression as E=log(CP100K+1).

Applicants reported the following QC metrics: number of total reads per library sample, sequencing saturation (fraction of reads originating from an already-observed UMI as reported by Cell Ranger count), total recovered cells or nuclei, number of reads per cell or nucleus, number of UMIs per cell or nucleus, number of genes detected per cell or nucleus, fraction of UMIs in a cell or nucleus aligned to mitochondrial genes, fraction of droplets estimated to contain only ambient RNA (“empty drops”), fraction of cell or nucleus doublets, the number of detected cell types, and the pattern of copy number aberration (CNA) for malignant cells. For a subset of samples, Applicants also calculated the UMI saturation for each cell or nucleus by subsampling from the total number of sequencing reads in the cell or nucleus²⁶, the number of cells or nuclei per detected cell type, and the estimated level of ambient RNA in droplets containing cells.

Applicants predicted droplets containing only ambient RNA and no cells using EmptyDrops, with the retain parameter set by the knee of the curve in the barcode rank plot (cell barcodes ranked by their total UMIs)¹⁰. Applicants predicted potential doublets using Scrublet with expected_doublet_rate=0.0611. Applicants estimated the levels of ambient RNA using SoupX and a set of cell-type specific marker genes¹⁸. Importantly, Applicants flagged the doublets and empty drops and retained them in their analysis, instead of immediately filtering them out. Droplets that appear to contain doublets or empty drops can arise from many different effects, such as cellular differentiation or insufficient sequencing, and by carrying them through the analysis, potential doublets or empty drops can be more clearly interpreted in the context of the full dataset.

Dimensionality reduction, clustering, and visualization. For each tumor sample, Applicants analyzed the filtered expression matrix to identify cell subsets, as previously described^{27,28}. Applicants chose highly variable genes with a z-score cutoff of 0.5²⁹, centered and scaled the expression of each gene to have a mean of zero and standard deviation of one, and performed dimensionality reduction on the variable genes using principal component analysis (PCA). Applicants used the top 50 principal components (PCs) as input to Louvain graph-based clustering, with the resolution parameter set to 1.3. For each cluster of cells, Applicants identified cluster-specific differentially expressed genes using the following tests: an AUC classifier, Welch's t-test, and Fisher's exact test. For tests that returned a p-value, Applicants controlled the false discovery rate at 5% with the Benjamini-Hochberg procedure³⁰. Applicants visualized gene expression and clustering results by embedding cells or nuclei profiles in a Uniform Manifold Approximation and Projection (UMAP)³¹ of the top 50 PCs, with min_dist=0.5, spread=1.0, the number of neighbors=15, and the Euclidean distance metric.

Annotating cell subsets. For each cell subset identified by clustering, Applicants assigned a cell type from the malignant, parenchymal, stromal, and immune compartments of the tumor microenvironment using a combination of differentially expressed genes, known gene signatures, and SingleR³², an automated annotation package. When running SingleR, only cell types assigned to 30 or more cells were considered. When scoring cells for the expression of known gene signatures, Applicants used the AddModuleScore function in Seurat (v2.3.4)²⁴. Applicants note that overlapping expression programs between T cells and NK cells make these cell types sometimes more difficult to accurately identify.

Applicants identified the malignant cells by inferring chromosomal copy number aberrations (CNAs) from the gene-expression data using inferCNV (v1.1.0)³³. On a sample-by-sample basis, Applicants used the immune and endothelial cells as a healthy reference to estimate CNAs in the malignant cells. Applicants created the count matrix file and annotation file for inferCNV by randomly subsetting the counts data to sample at most 2,000 cells or nuclei. Applicants created a gene ordering file from the human GRCh38 assembly, which contains the chromosomal start and end positions for each gene. To run inferCNV, Applicants used a cutoff of 0.1 for the minimum average read counts per gene among reference cells or nuclei, clustered according to the annotated cell types, denoised our output, ran an HMM to predict CNA level, implemented inferCNV's i6 HMM model, and requested 8 threads for parallel steps.

Comparing single cell and single nucleus RNA-Seq data. To compare profiles between single cell and single nucleus RNA-Seq data collected from the same sample, Applicants used a batch-correction approach.

For the batch correction approach, Applicants performed batch correction using canonical correlation analysis (CCA) as implemented in Seurat (v2.3.4)²⁴. Applicants selected 1,500 genes that were variable across both the cell and nucleus data, used those genes as input to RunCCA to compute the first 20 canonical components, and aligned the first 12 canonical components with AlignSubspace. The aligned canonical components represent a co-embedding of the cell and nucleus data, and Applicants carried out clustering in this dimensionality-reduced space using FindClusters.

Software and data availability. Applicants implemented all major analysis steps, from FASTQ files to identifying cell subsets, in pipelines executed in a Cloud environment. Applicants named this collection of pipelines scCloud, which may be executed in both a Cloud-based environment and a local, python environment.

Pipelines were written in the Workflow Description Language (WDL) and run on Cromwell in the Terra Cloud platform (https://app.terra.bio/), and data was stored in Google Cloud Plaform storage buckets. Applicants wrote two WDL workflows: cellranger_workflow, a wrapper for running Cell Ranger mkfastq and count, and scCloud, a novel, fast, and scalable analysis pipeline for single cell and single nucleus RNA-Seq data. All analysis workflows will be publicly available through https://github.com/klarman-cell-observatory/scCloud.

Applicants ran additional quality control steps, cell-subset annotations, and protocol comparison steps in R (v3.5) by converting the single-cell AnnData objects from scCloud into Seurat objects. An example script for this analysis will be made available at https://github.com/klarman-cell-observatory/HTAPP-Pipelines.

Raw data will be available in the controlled access repository DUOS (https://duos.broadinstitute.org/#/home).

### REFERENCES

1. Jerby-Arnon, L., et al. A Cancer Cell Program Promotes T Cell Exclusion and Resistance to Checkpoint Blockade. Cell 175, 984-997.e924 (2018).
2. Puram, S. V., et al. Single-Cell Transcriptomic Analysis of Primary and Metastatic Tumor Ecosystems in Head and Neck Cancer. Cell 171, 1611-1624 e1624 (2017).
3. Filbin, M. G., et al. Developmental and oncogenic programs in H3K27M gliomas dissected by single-cell RNA-seq. Science 360, 331-335 (2018).
4. Venteicher, A. S., et al. Decoupling genetics, lineages, and microenvironment in IDH-mutant gliomas by single-cell RNA-seq. Science 355(2017).
5. Tirosh, I., et al. Single-cell RNA-seq supports a developmental hierarchy in human oligodendroglioma. Nature 539, 309-313 (2016).
6. Cieslik, M. & Chinnaiyan, A. M. Cancer transcriptome profiling at the juncture of clinical translation. Nat Rev Genet 19, 93-109 (2018).
7. Habib, N., et al. Div-Seq: Single-nucleus RNA-Seq reveals dynamics of rare adult newborn neurons. Science 353, 925-928 (2016).
8. Habib, N., et al. Massively parallel single-nucleus RNA-seq with DroNc-seq. Nat Methods (2017).
9. Gaublomme, J. T., et al. Nuclei multiplexing with barcoded antibodies for single-nucleus genomics. Nat Commun 10, 2907 (2019).
10. Lun, A. T. L., et al. EmptyDrops: distinguishing cells from empty droplets in droplet-based single-cell RNA sequencing data. Genome Biol 20, 63 (2019).
11. Wolock, S. L., Lopez, R. & Klein, A. M. Scrublet: Computational Identification of Cell Doublets in Single-Cell Transcriptomic Data. Cell Syst 8, 281-291 e289 (2019).
12. Li, B., Gould, J., Rosen, Y., Rozenblatt-Rosen, O. & Regev, A. https://github.com/klarman-cell-observatory/KCO. (2019).
13. Al-Hajj, M., Wicha, M. S., Benito-Hernandez, A., Morrison, S. J. & Clarke, M. F. Prospective identification of tumorigenic breast cancer cells. Proc Natl Acad Sci U S A 100, 3983-3988 (2003).
14. McDivitt, R. W., Stone, K. R. & Meyer, J. S. A method for dissociation of viable human breast cancer cells that produces flow cytometric kinetic information similar to that obtained by thymidine labeling. Cancer Res 44, 2628-2633 (1984).
15. Neftel, C., et al. An Integrative Model of Cellular States, Plasticity, and Genetics for Glioblastoma. Cell (2019).
16. Quatromoni, J. G., et al. An optimized disaggregation method for human lung tumors that preserves the phenotype and function of the immune cells. J Leukoc Biol 97, 201-209 (2015).
17. Burgstaller, G., et al. The instructive extracellular matrix of the lung: basic composition and alterations in chronic lung disease. Eur Respir J 50(2017).
18. Young, M. D. & Behjati, S. SoupX removes ambient RNA contamination from droplet based single cell RNA sequencing data. 303727 (2018).
19. Stewart, E., et al. Development and characterization of a human orthotopic neuroblastoma xenograft. Dev Biol 407, 344-355 (2015).
20. Stewart, E., et al. Orthotopic patient-derived xenografts of paediatric solid tumours. Nature 549, 96-100 (2017).
21. Hermansen, J. U., Tjonnfjord, G. E., Munthe, L. A., Tasken, K. & Skanland, S. S. Cryopreservation of primary B cells minimally influences their signaling responses. Sci Rep 8, 17651 (2018).
22. Guillaumet-Adkins, A., et al. Single-cell transcriptome conservation in cryopreserved cells and tissues. Genome Biol 18, 45 (2017).
23. Gao, R., et al. Nanogrid single-nucleus RNA sequencing reveals phenotypic diversity in breast cancer. Nat Commun 8, 228 (2017).
24. Butler, A., Hoffman, P., Smibert, P., Papalexi, E. & Satija, R. Integrating single-cell transcriptomic data across different conditions, technologies, and species. Nature biotechnology 36, 411-420 (2018).
25. Bakken, T. E., et al. Single-nucleus and single-cell transcriptomes compared in matched cortical cell types. PLoS One 13, e0209648 (2018).
26. Wallrapp, A., et al. The neuropeptide NMU amplifies ILC2-driven allergic lung inflammation. Nature 549, 351-356 (2017).
27. Shekhar, K., et al. Comprehensive Classification of Retinal Bipolar Neurons by Single-Cell Transcriptomics. Cell 166, 1308-1323 e1330 (2016).
28. Wolf, F. A., Angerer, P. & Theis, F. J. SCANPY: large-scale single-cell gene expression data analysis. Genome Biol 19, 15 (2018).
29. Macosko, E. Z., et al. Highly Parallel Genome-wide Expression Profiling of Individual Cells Using Nanoliter Droplets. Cell 161, 1202-1214 (2015).
30. Benjamini, Y., and Yosef Hochberg. “Controlling the false discovery rate: a practical and powerful approach to multiple testing”. Journal of the Royal statistical society: series B (Methodological) 57.1 289-300 (1995).
31. Leland McInnes, J. H., James Melville. UMAP: Uniform Manifold Approximation and Projection for Dimension Reduction. (eprint arXiv:1802.03426, 2018).
32. Aran, D., et al. Reference-based analysis of lung single-cell sequencing reveals a transitional profibrotic macrophage. Nat Immunol 20, 163-172 (2019).
33. Tickle T, T. I., Georgescu C, Brown M, Haas B inferCNV of the Trinity CTAT Project, https://github . com/broadinstitute/inferCNV. (2019).

### SUPPLEMENTAL REFERENCES

Benjamini, Y., and Hochberg, Y. (1995). Controlling the False Discovery Rate: A Practical and Powerful Approach to Multiple Testing. J. R. Stat. Soc. Series B Stat. Methodol. 57, 289-300.
Bondurand, N., and Southard-Smith, E. M. (2016). Mouse models of Hirschsprung disease and other developmental disorders of the enteric nervous system: Old and new players. Dev. Biol. 417, 139-157.
Brookes, S. J., Steele, P. A., and Costa, M. (1991a). Identification and immunohistochemistry of cholinergic and non-cholinergic circular muscle motor neurons in the guinea-pig small intestine. Neuroscience 42, 863-878.
Brookes, S. J., Steele, P. A., and Costa, M. (1991b). Calretinin immunoreactivity in cholinergic motor neurones, interneurones and vasomotor neurones in the guinea-pig small intestine. Cell Tissue Res. 263, 471-481.
Butler, A., Hoffman, P., Smibert, P., Papalexi, E., and Satija, R. (2018). Integrating single-cell transcriptomic data across different conditions, technologies, and species. Nat. Biotechnol. 36, 411.
Chang, D., Nalls, M. A., Hallgrímsdóttir, I. B., Hunkapiller, J., van der Brug, M., Cai, F., International Parkinson's Disease Genomics Consortium, 23andMe Research Team, Kerchner, G. A., Ayalon, G., et al. (2017). A meta-analysis of genome-wide association studies identifies 17 new Parkinson's disease risk loci. Nat. Genet. 49, 1511-1516.
Drokhlyansky, E., Göz Aytürk, D., Soh, T. K., Chrenek, R., O'Loughlin, E., Madore, C., Butovsky, O., and Cepko, C. L. (2017). The brain parenchyma has a type I interferon response that can limit virus spread. Proc. Natl. Acad. Sci. U. S. A. 114, E95-E104.
Finak, G., McDavid, A., Yajima, M., Deng, J., Gersuk, V., Shalek, A. K., Slichter, C. K., Miller, H. W., McElrath, M. J., Prlic, M., et al. (2015). MAST: a flexible statistical framework for assessing transcriptional changes and characterizing heterogeneity in single-cell RNA sequencing data. Genome Biol. 16, 278.
Furness, J. B. (2012). The enteric nervous system and neurogastroenterology. Nat. Rev. Gastroenterol. Hepatol. 9, 286-294.
Furness, J. B., Costa, M., Rökaeus, A., McDonald, T. J., and Brooks, B. (1987). Galanin-immunoreactive neurons in the guinea-pig small intestine: their projections and relationships to other enteric neurons. Cell Tissue Res. 250, 607-615.
Grider, J. R. (1994). Interplay of somatostatin, opioid, and GABA neurons in the regulation of the peristaltic reflex. Am. J. Physiol. 267, G696-G701.
Grider, J. R. (2003). Neurotransmitters mediating the intestinal peristaltic reflex in the mouse. J. Pharmacol. Exp. Ther. 307, 460-467.
Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339.
Habib, N., Li, Y., Heidenreich, M., Swiech, L., Avraham-Davidi, I., Trombetta, J. J., Hession, C., Zhang, F., and Regev, A. (2016). Div-Seq: Single-nucleus RNA-Seq reveals dynamics of rare adult newborn neurons. Science 353, 925-928.
Habib, N., Avraham-Davidi, I., Basu, A., Burks, T., Shekhar, K., Hofree, M., Choudhury, S. R., Aguet, F., Gelfand, E., Ardlie, K., et al. (2017). Massively parallel single-nucleus RNA-seq with DroNc-seq. Nat. Methods 14, 955-958.
Lasrado, R., Boesmans, W., Kleinjung, J., Pin, C., Bell, D., Bhaw, L., McCallum, S., Zong, H., Luo, L., Clevers, H., et al. (2017). Lineage-dependent spatial and functional organization of the mammalian enteric nervous system. Science 356, 722-726.
Levine, J. H., Simonds, E. F., Bendall, S. C., Davis, K. L., Amir, E.-A. D., Tadmor, M. D., Litvin, O., Fienberg, H. G., Jager, A., Zunder, E. R., et al. (2015). Data-Driven Phenotypic Dissection of AML Reveals Progenitor-like Cells that Correlate with Prognosis. Cell 162, 184-197.
Lewis, A. E., Vasudevan, H. N., O'Neill, A. K., Soriano, P., and Bush, J. O. (2013). The widely used Wnt1-Cre transgene causes developmental phenotypes by ectopic activation of Wnt signaling. Dev. Biol. 379, 229-234.
Li, B., and Dewey, C. N. (2011). RSEM: accurate transcript quantification from RNA-Seq data with or without a reference genome. BMC Bioinformatics 12, 323.
Linkert, M., Rueden, C. T., Allan, C., Burel, J.-M., Moore, W., Patterson, A., Loranger, B., Moore, J., Neves, C., Macdonald, D., et al. (2010). Metadata matters: access to image data in the real world. J. Cell Biol. 189, 777-782.
Lopez, R., Regier, J., Cole, M. B., Jordan, M. I., and Yosef, N. (2018). Deep generative modeling for single-cell transcriptomics. Nat. Methods 15, 1053-1058.
Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nat. Neurosci. 13, 133-140.
Matsuoka, T., Ahlberg, P. E., Kessaris, N., Iannarelli, P., Dennehy, U., Richardson, W. D., McMahon, A. P., and Koentges, G. (2005). Neural crest origins of the neck and shoulder. Nature 436, 347-355.
Mo, A., Mukamel, E. A., Davis, F. P., Luo, C., Henry, G. L., Picard, S., Urich, M. A., Nery, J. R., Sejnowski, T. J., Lister, R., et al. (2015). Epigenomic Signatures of Neuronal Diversity in the Mammalian Brain. Neuron 86, 1369-1384.
Pietzsch, T., Preibisch, S., Tomancák, P., and Saalfeld, S. (2012). ImgLib2-generic image processing in Java. Bioinformatics 28, 3009-3011.
Ramilowski, J. A., Goldberg, T., Harshbarger, J., Kloppmann, E., Lizio, M., Satagopam, V. P., Itoh, M., Kawaji, H., Carninci, P., Rost, B., et al. (2015). A draft network of ligand-receptor-mediated multicellular signalling in human. Nat. Commun. 6, 7866.
Rossi, J., Luukko, K., Poteryaev, D., Laurikainen, A., Sun, Y. F., Laakso, T., Eerikainen, S., Tuominen, R., Lakso, M., Rauvala, H., et al. (1999). Retarded growth and deficits in the enteric and parasympathetic nervous system in mice lacking GFR alpha2, a functional neurturin receptor. Neuron 22, 243-252.
Sanders, S. J., He, X., Willsey, A. J., Ercan-Sencicek, A. G., Samocha, K. E., Cicek, A. E., Murtha, M. T., Bal, V. H., Bishop, S. L., Dong, S., et al. (2015). Insights into Autism Spectrum Disorder Genomic Architecture and Biology from 71 Risk Loci. Neuron 87, 1215-1233.
Sang, Q., and Young, H. M. (1996). Chemical coding of neurons in the myenteric plexus and external muscle of the small and large intestine of the mouse. Cell Tissue Res. 284, 39-53.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat. Methods 9, 676-682.
Schindelin, J., Rueden, C. T., Hiner, M. C., and Eliceiri, K. W. (2015). The ImageJ ecosystem: An open platform for biomedical image analysis. Mol. Reprod. Dev. 82, 518-529.
Schneider, C. A., Rasband, W. S., and Eliceiri, K. W. (2012). NIH Image to ImageJ: 25 years of image analysis. Nat. Methods 9, 671-675.
Schuchardt, A., D'Agati, V., Pachnis, V., and Costantini, F. (1996). Renal agenesis and hypodysplasia in ret-k-mutant mice result from defects in ureteric bud development. Development 122, 1919-1929.
Shekhar, K., Lapan, S. W., Whitney, I. E., Tran, N. M., Macosko, E. Z., Kowalczyk, M., Adiconis, X., Levin, J. Z., Nemesh, J., Goldman, M., et al. (2016). Comprehensive Classification of Retinal Bipolar Neurons by Single-Cell Transcriptomics. Cell 166, 1308-1323.e30.
Smillie, C. S., Biton, M., Ordovas-Montanes, J., Sullivan, K. M., Burgin, G., Graham, D. B., Herbst, R. H., Rogel, N., Slyper, M., Waldman, J., et al. (2018). Rewiring of the cellular and inter-cellular landscape of the human colon during ulcerative colitis.
Vento-Tormo, R., Efremova, M., Botting, R. A., Turco, M. Y., Vento-Tormo, M., Meyer, K. B., Park, J.-E., Stephenson, E., Polanski, K., Goncalves, A., et al. (2018). Single-cell reconstruction of the early maternal-fetal interface in humans. Nature 563, 347-353.
Wiese, C. B., Fleming, N., Buehler, D. P., and Southard-Smith, E. M. (2013). A Uchl1 -Histone2BmCherry:GFP-gpi BAC transgene for imaging neuronal progenitors. Genesis 51, 852-861.
Young, H. M., Furness, J. B., and Povey, J. M. (1995). Analysis of connections between nitric oxide synthase neurons in the myenteric plexus of the guinea-pig small intestine. J. Neurocytol. 24, 257-263.

### Example 25

### Expanded Single-Cell and Single-Nucleus RNA-Seq Toolbox for Processing Tumors

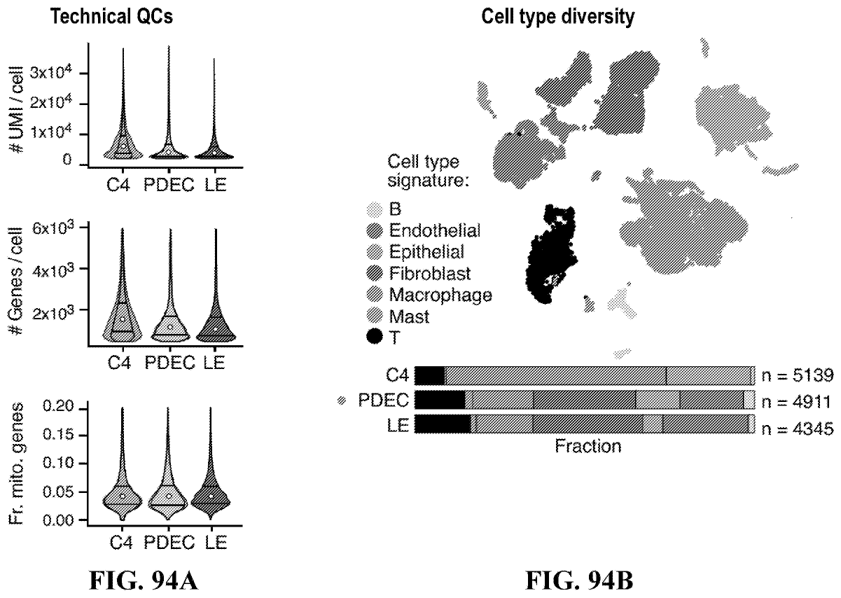

Applicants began by processing fresh tissue. To choose the best performing dissociation method, Applicants apportioned large tumor specimens into smaller pieces (˜0.5-2 cm), dissociating each piece with a different protocol for fresh tissue dissociation. Collagenase 4 is NSCLC-C4, PDEC is a mixture of Pronase, Dispase, Elastase, and Collagenases A and 4. LE consists of Liberase™ and Elastase. Each of these was prepared in combination with DNase I. While the QCs looked similar (FIG. 94A) we see that each protocol results in a different proportion of cell types (FIG. 94B). For example C4 does not recover mast cells, endothelial cells, or fibroblasts, while the other two protocols do. In this case Applicants chose the PDEC protocol for future processing and also used this protocol for processing fresh normal lung samples.

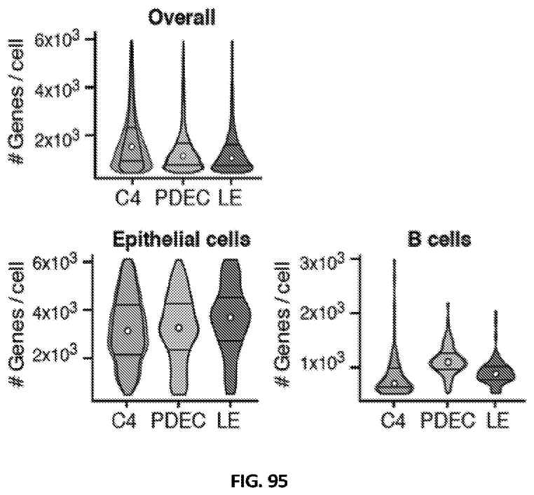

Applicants found that the C4 protocol has the highest number of genes detected per cell overall. However, looking within cell types, Applicants found that similar number of cells were recovered across all three protocols, with C4 having greater cell type proportion of epithelial cells and macrophages. These cells are typically larger, have more starting RNA and more genes detected per cell, and so overall have the highest number genes detected per cell. Because cell type proportions may vary between protocols, and the number of detected genes (and other metrics) varies between cell types, it is important to also assess cell-type specific QCs when choosing a protocol. For example, while C4 has the highest number of genes detected overall per cell, LE has the greatest number of genes detected per cell in epithelial cells and PDEC has the greatest number of genes detected per cell in B cells (FIG. 95).

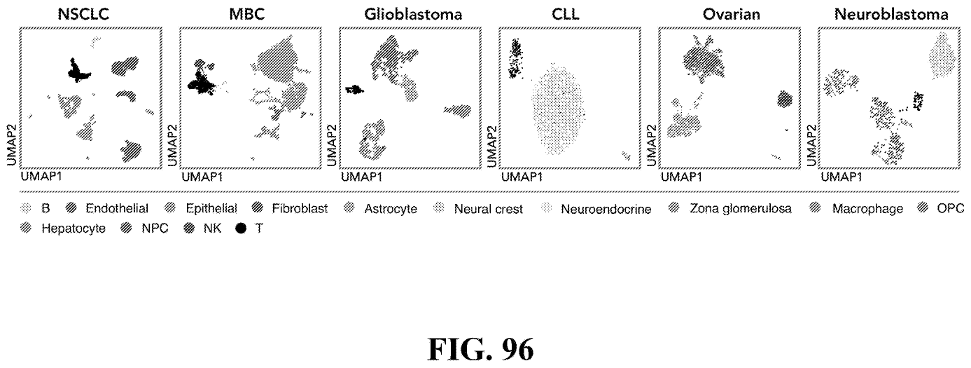

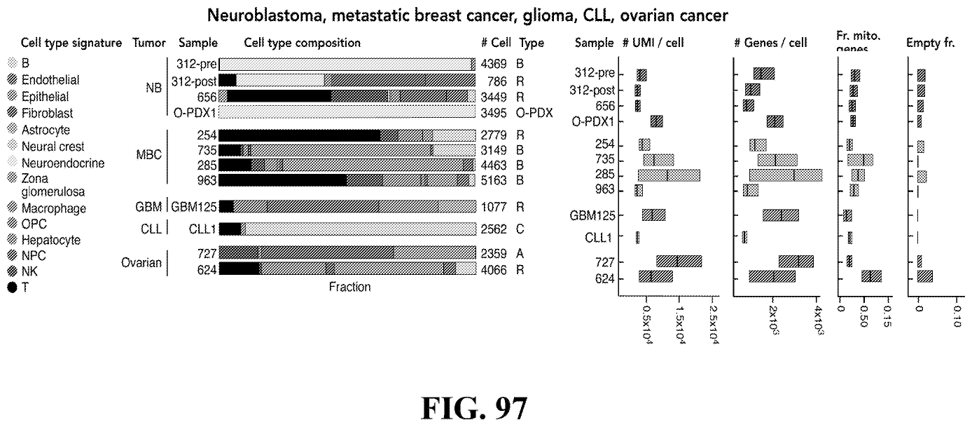

Overall, Applicants processed five types of fresh tumors: non-small cell lung carcinoma (NSCLC), metastatic breast cancer (MBC), ovarian cancer, glioblastoma (GBM), and neuroblastoma, as well as a cryopreserved non-solid, chronic lymphocytic leukemia (CLL) (FIG. 96). Applicants measured QCs for all tissue types, looked at cell proportions and chose a recommended protocol for each tumor type (FIG. 97). While fresh sample processing generally works well, it has several limitations. First, one has to tailor cell dissociation to tumor type (cell type and ECM components). Processing is also time sensitive. Changes in gene expression are also common and there is loss of sensitive cells during dissociation. Moreover, there is no possibility for multiplexing.

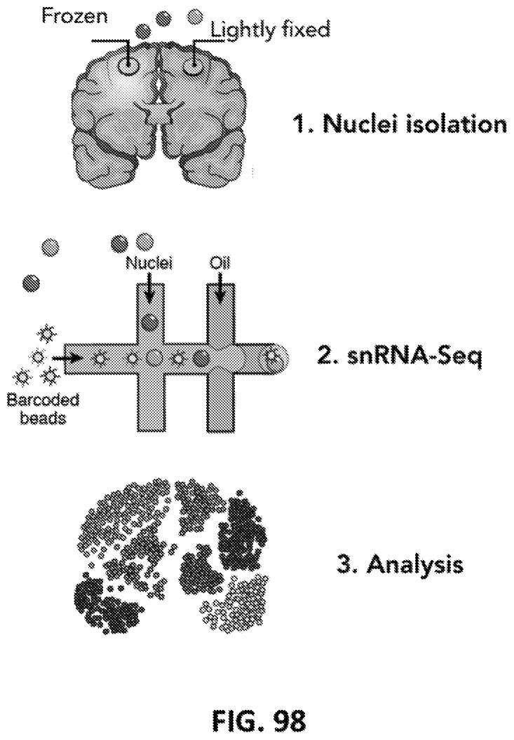

To address these limitations Applicants previously developed a single-nuclei (snRNA-seq) method for profiling expression in single nuclei (FIG. 98). See also WO 2017/0164936, the entirety of which is incorporated by reference herein. snRNA-seq has several advantages. For example, it does not require cell dissociation, can use frozen or lightly fixed tissue, decouples collection from processing, can use banked samples, allows early pooling within and across donors, and allows for massively parallel implementation.

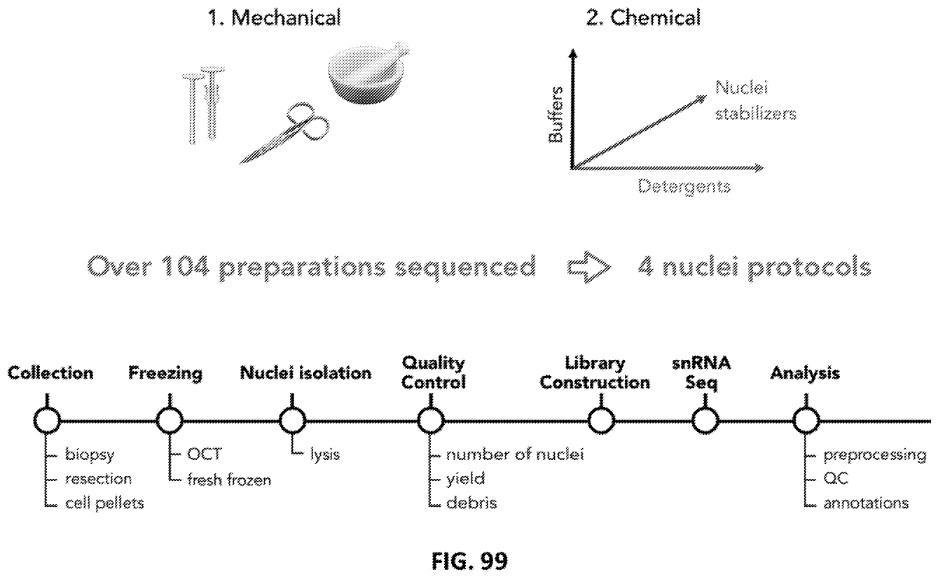

Application of the protocol tumor tissues required some modification. Buffers, detergent, and force were optimized with over 104 preparations and Applicants developed a nuclei processing toolbox to quickly and effectively profile frozen tissues. The best general approach was testing four different nucleus isolation buffers, three of which were very similar to each other apart from the detergent and the original buffer EZ (FIG. 99).

To understand the basis for performance differences among nuclei preparations, Applicants compared nuclei structure between the new and published preparations for snRNA-seq electron microscopy. The published methods yielded isolated intact nuclei. In contrast, CST preserved not only the nuclear envelope, but also the ribosomes on the outer nuclear membrane. Applicants thus termed this method RAISIN (Ribosomes And Intact SIngle Nucleus) RNA-seq. TST maintained both the rough ER and its attached ribosomes on the outer nuclear membrane, Applicants thus termed this method, INNER Cell (INtact Nucleus and Endoplasmic Reticulum from a single Cell). Consistent with the TEM results, both RAISIN RNA-seq and INNER Cell RNA-seq yielded higher exon:intron ratios than the published methods (41% and 64% increases, respectively), suggesting greater recovery of mRNA relative to pre-mRNA.

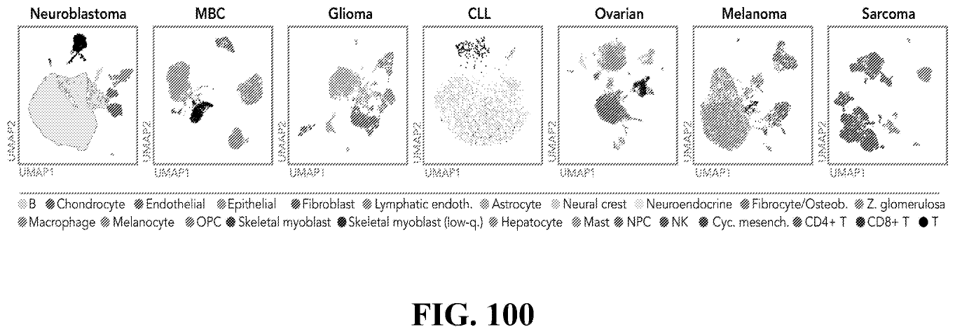

With this toolbox in hand, Applicants tested it on tumors, starting with Neuroblastoma. Applicants observed a similar number of nuclei recovered across protocols, but different cell type proportions, in particular in the T cells, fibroblasts, and zona glomerulosa. Applicants did observe that the EZ buffer did not perform as well. Applicants could apply this across many tumor types, including Neuroblastoma, MBC, glioma, CLL, ovarian cancer, melanoma, and sarcoma (FIG. 100).

Looking again at QCs and proportions, Applicants observed that in most cases the TST buffer outperforms the other buffers—so while it has more mitochondrial reads—in most cases it detects more immune cells. EZ performed the least well among these tumor types. Applicants next wanted to compare sc/sn RNA-Seq on the same tumor sample, so they took two pieces. One freshly processed by scRNA-seq, one frozen and processed by snRNA-seq. Applicants combined the two datasets, clustered them to identify cell types, and visualized them in UMAP embedding. Applicants observed more T cells in scRNA-seq, more neural crest (cell of origin), endothelial cell in snRNA-seq. Each method has different biases to types of cells recovered.

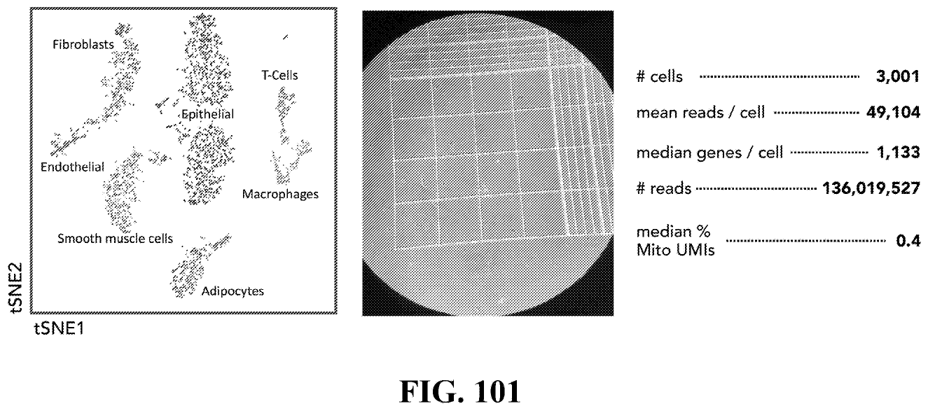

Lastly, Applicants wanted to test frozen pre-cancer samples, so a frozen DCIS sample and profile was run using the nuclei toolbox. After analysis, Applicants obtained good QC metrics and could detect several cell types—including two clusters of epithelial cells, immune cells, endothelial cells, and fibroblasts (FIG. 101).

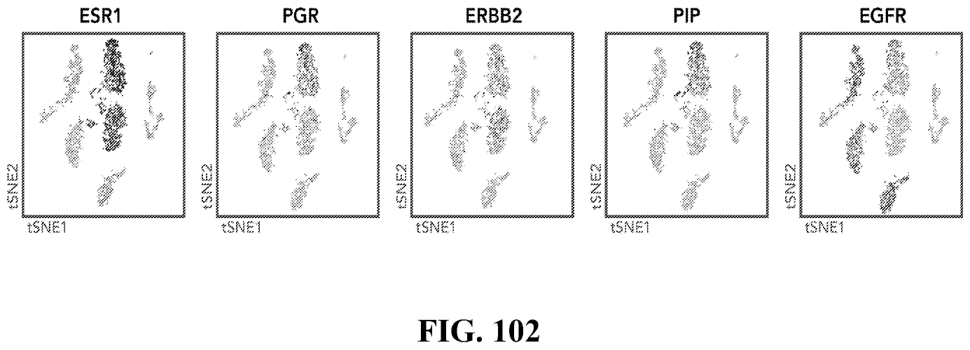

Applicants also looked at specific breast cancer markers, such as estrogen receptor, progesterone receptor, and ERBB2-HER2. Applicants observed PIP-prolactin induced protein, a biomarker for early stage BC (FIG. 102).

In summary, to choose the protocol for the cancer type in question, it is best to compare two to three protocols in parallel on the same tissue sample. It is also advisable to check all QCs and if possible, compare sc/snRNA-seq on the same tissue sample. The “best” protocol depends on the biological question: one must choose the protocol that recovers the greatest cellular diversity (for atlas), and it also depends on whether one is looking at deletion or enrichment of markers.

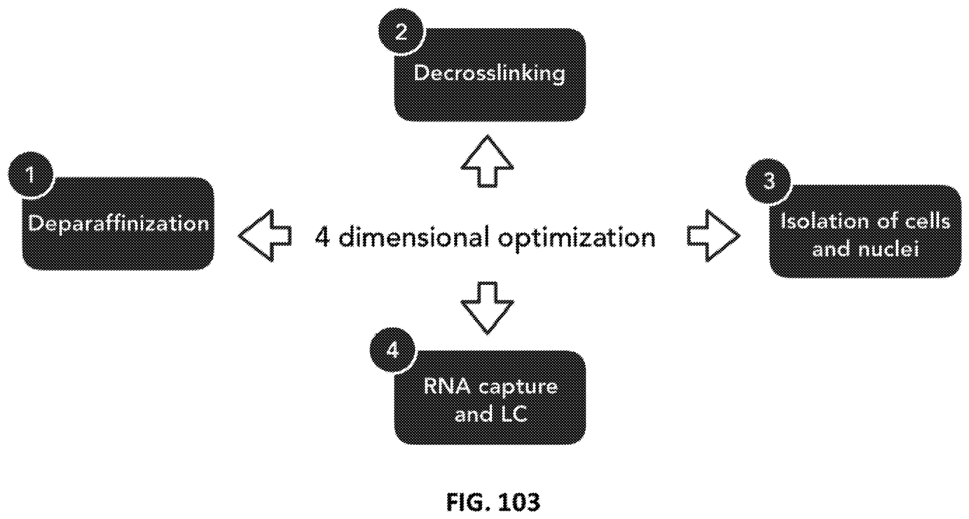

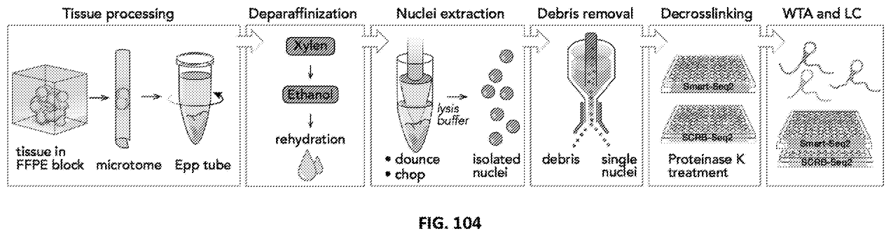

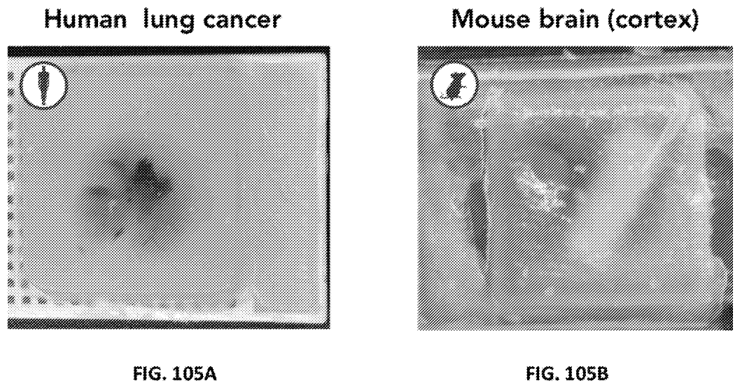

To optimize the protocol on FFPE tissues, it is necessary to focus on 4 main steps in the protocol: 1) deparafinization—get rid of the FFPE; 2) decrosslinking; 3) isolation of nuclei; and 4) capture RNA and Library construction (FIG. 103). Some steps may be tissue specific. All steps for the workflow were optimized, as illustrated in FIG. 104. In terms of samples Applicants focused on mouse brain (FIG. 105B). Many methods are developed using this tissue because it has a lot of RNA. All the FFPE blocks were made fresh and processed quickly. Applicants are now also working on getting scrolls from lung cancer patients (FIG. 105A).

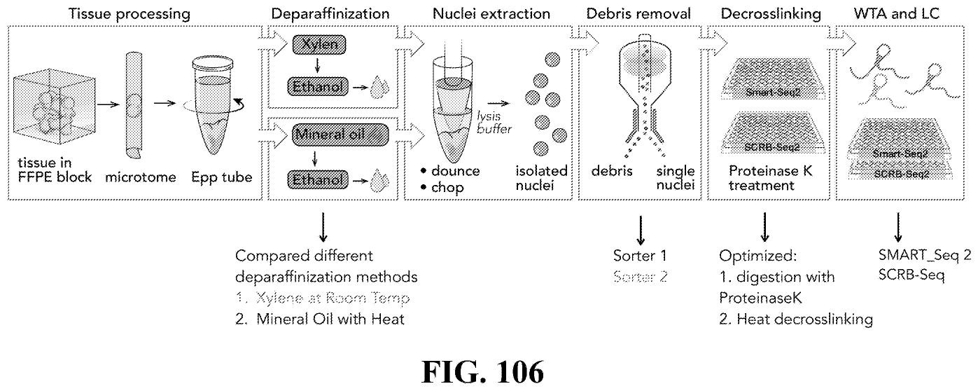

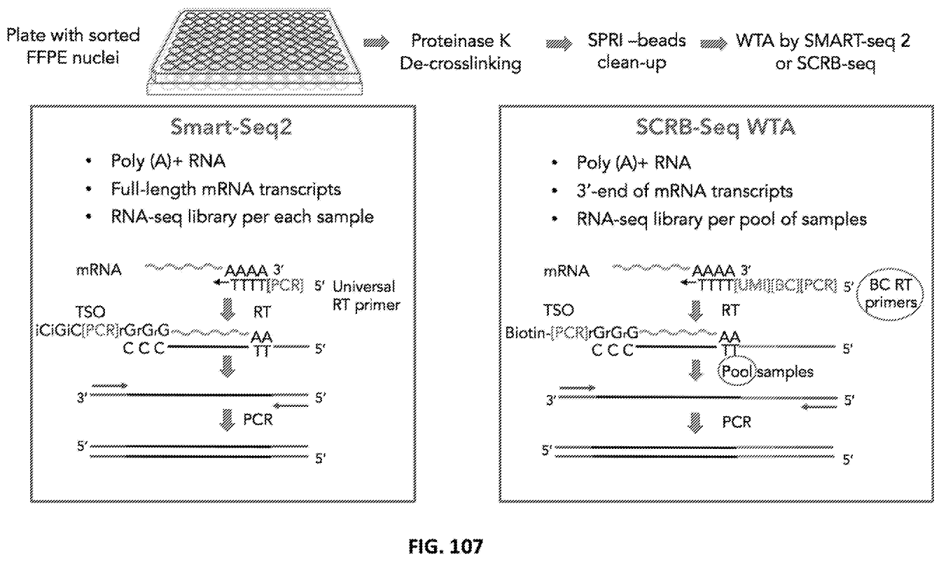

During optimization, Applicants compared different deparaffinization methods. Applicants optimized digestion with ProteinaseK and heat decrosslinking. Applicants also used two different library construction (LC) methods—SCRB-Seq and Smart-seq2 (FIG. 106). Both methods are poly A based, but the main difference is that SMART-Seq2 generates full length transcripts, while SCRB-Seq you get the 3′ end of mRNA transcripts. Another difference is that in SMART-Seq2 each cell is processed by itself, while in SCRB-Seq there is early pooling of cells as a barcode is added at the cDNA stage (FIG. 107).

SCRB-Seq Whole Transcriptome Amplification (WTA) was tested for FFPE because it allows for a high level of multiplexing—barcoding of samples started at reverse transcription (RT). SCRB-Seq has high sensitivity because it amplifies pools of samples (there is more PCR template present). It uses unique molecular identifiers (UMIs) to detect and quantify unique mRNA transcripts. The cost of constructing sequencing libraries is low—with one library per pool of samples.

Applicants made the following modifications to the SCRB-Seq protocol for FFPE. RT reaction was done with barcode primers in SMART-Seq2 reaction conditions with less expensive template switching oligos, post-RT PCR conditions were optimized, and cDNA-seq library construction was improved.

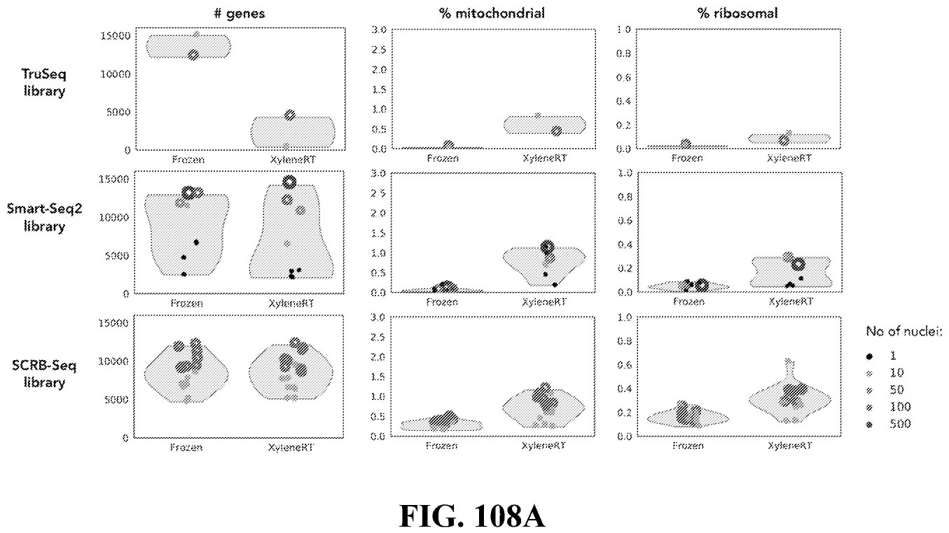

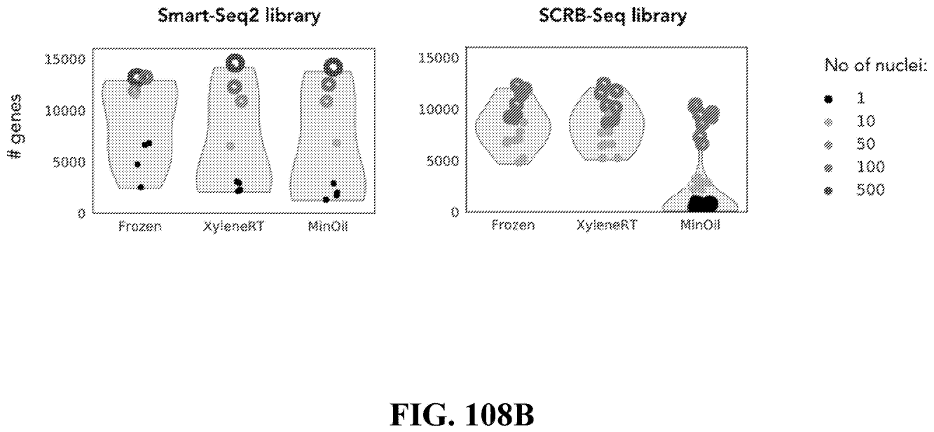

Applicants compared the two methods using the chosen extraction buffer (Xylene RT), used a frozen sample as a positive control and used had a varying number of nuclei. When Applicants looked at the QC they observed that in both SMART-Seq2 and SCRB-Seq libraries a significant number of genes can be detected. Also, the mitochondrial and ribosomal fractions are considerably low (FIG. 108A).

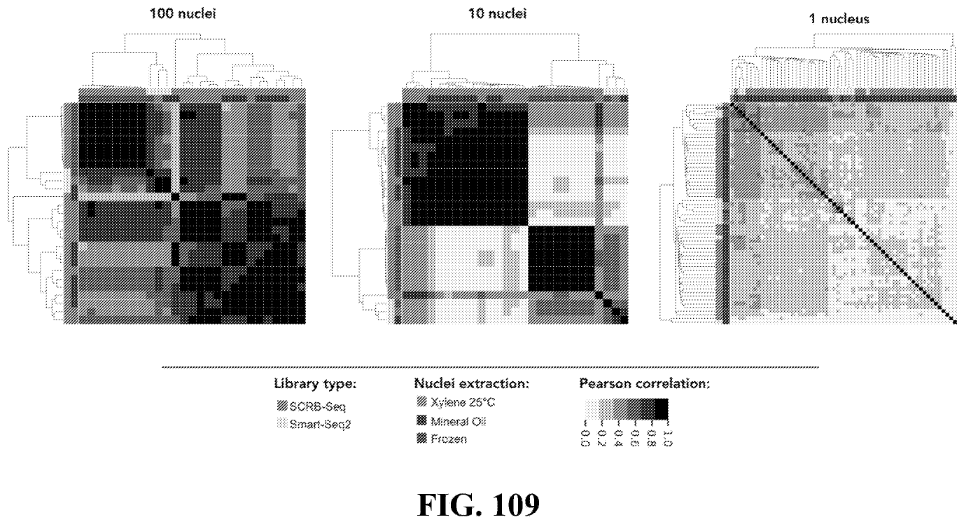

Applicants then looked at correlation of expression across library preps, nuclei extraction method and number of nuclei. Applicants observed that when they processed 100 nuclei—there was good correlation between the different frozen samples and between the different FFPE samples. For the FFPE samples, even if the prep was not the same, he samples still clustered together and Applicants also saw that there was a good correlation between FFPE and frozen samples. As expected, the correlation goes down with the numbers of nuclei tested—since cortex mouse is a complex tissue with many cell types. Correlation across preps was as follows: 100/10 frozen nuclei preps tend to cluster together and 100/10 FFPE nuclei preps tend to cluster together but one can see good correlation between frozen and FFPE at 100 nuclei (precision=XXX). Lower correlation was observed at 1 nucleus since brain cortex has many cell types and states and data are sparse (FIG. 109).

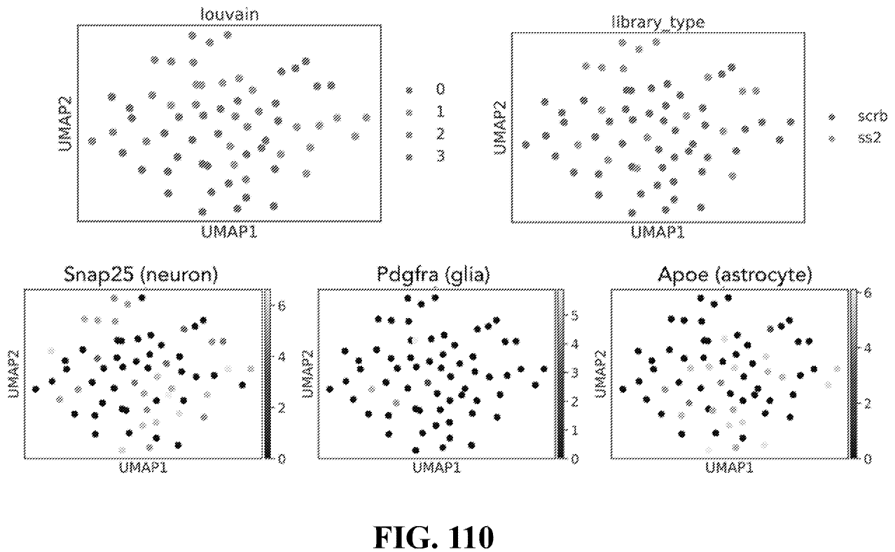

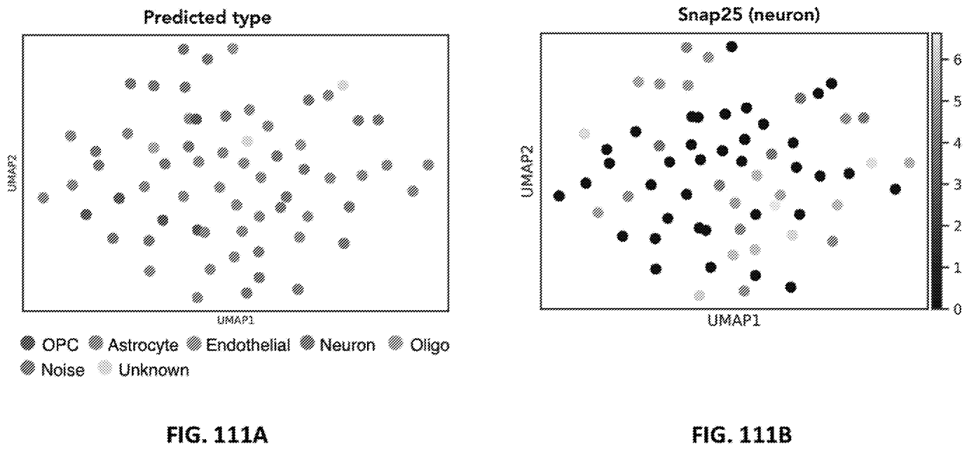

Applicants next tried to cluster the single nuclei and did not observe clear clusters—as the number of cells profiled was too low. However, when looking at known mouse marker genes—expression of specific markers for neurons, glia, and astrocytes in the single cells is observerd (FIG. 110). Moreover, Applicants could use data generated for single cells in mouse cortex brain (from the BICCN) and predict cell types from the FFPE data. Accordingly, Applicants were able to predict several cell types at good accuracy (FIGS. 111A, 111B). Applicants used 2,006 genes detected found in both the 10× data (mouse BICCN) and the single nuclei FFPE data to train classifier. First, Applicants split the 10× data in half (train and test data sets), then they ran the classifier on train set, and used it to predict cell type labels on test set.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of recovering nuclei or whole cells from a formalin-fixed paraffin-embedded (FFPE) tissue comprising:
a. dissolving paraffin from a FFPE tissue sample in a solvent, preferably the solvent is selected from the group consisting of xylene and mineral oil, wherein the tissue is dissolved at a temperature between 4 C to 90 C, preferably room temperature (20 to 25 C) for recovering whole cells and 90 C for recovering nuclei;
b. rehydrating the tissue using a gradient of ethanol from 100% to 0% ethanol (EtOH);
c. transferring the rehydrated tissue to a volume of a first buffer comprising a buffering agent, a detergent and an ionic strength between 100 mM and 200 mM, optionally the first buffer comprises protease inhibitors or proteases and/or BSA;
d. chopping or dounce homogenizing the tissue in the buffer; and
e. removing debris by filtering and/or FACS sorting.

2. The method of claim 1, further comprising isolating nuclei or cell types by FACS sorting.

3. The method of claim 1, wherein dissolving paraffin from a FFPE tissue sample, comprises incubating at least one time in xylene, at room temperature (RT), for about 10 minutes each, and wherein xylene is removed at each change.

4. The method of claim 3, further comprising washing the tissue at least two times with xylene for about 10 min each, wherein the washes are performed at room temperature (RT), 90 C, or at least one time at room temperature (RT) and at least one time at 90 C, wherein xylene is removed at each change.

5. The method of claim 1, wherein dissolving paraffin from a FFPE tissue sample, comprises incubating at least twice in about 5 ml xylene per 30-100 mg FFPE tissue sample, at room temperature, for about 10 minutes each, wherein xylene is removed at each change.

6. The method of claim 5, further comprising washing the tissue with xylene at 37 C for about 10 min.

7. The method of claim 6, further comprising cutting the tissue into two or more pieces and washing at least one piece of the tissue with xylene at 37 C for about 10 min.

8. The method of claim 1, wherein dissolving paraffin from a FFPE tissue sample, comprises incubating at least three times in xylene, at room temperature, for about 10 minutes each, and wherein xylene is removed at each change.

9. The method of claim 8, further comprising washing the tissue three additional times with xylene for about 10 min each, wherein the first wash is at room temperature and the second and third washes are at 90 C, and wherein xylene is removed at each change.

10. The method of claim 1, wherein rehydrating the tissue comprises a step gradient of ethanol (EtOH) and the tissue is incubated between 1 to 10 minutes at each step.

11. The method of claim 10, wherein the step gradient comprises incubating the tissue for about 2 minutes each in successive washes of 95%, 75%, and 50% ethanol (EtOH).

12. The method of any of the preceding claims, wherein after rehydrating the tissue the method further comprises placing the tissue samples on ice or on a device capable of maintaining the tissue between 4 and 10 C, wherein all subsequent steps are performed at a temperature between 4 and 10 C.

13. The method of any of the preceding claims, wherein after the step of dissolving paraffin from the tissue or rehydrating the tissue the method further comprises dividing the tissue, preferably in half.

14. The method of claim 1, wherein the first buffer comprises a detergent selected from the group consisting of NP40, CHAPS and Tween-20.

15. The method of claim 14, wherein the NP40 concentration is about 0.2%.

16. The method of claim 14, wherein the Tween-20 concentration is about 0.03%.

17. The method of claim 14, wherein the CHAPS concentration is about 0.49%.

18. The method of claim 1, wherein the first buffer is selected from the group consisting of CST, TST, NST and NSTnPo.

19. The method of claim 1, wherein after the step of chopping or dounce homogenizing the method further comprises centrifuging, preferably, the sample is centrifuged at about 500 g for about 5 min, and resuspending the sample in a second buffer comprising a buffering agent and an ionic strength between 100 mM and 200 mM, optionally the second buffer comprises protease inhibitors.

20. The method of claim 19, wherein the second buffer is ST, optionally comprising protease inhibitors.

21. The method of claim 1, wherein the sample is filtered through a 40 uM filter.

22. The method of claim 21, further comprising washing the filtered sample in the first buffer.

23. The method of claim 22, further comprising filtering the sample through a 30 uM filter.

24. The method of claim 1, wherein after the step of chopping or dounce homogenizing the method further comprises adding an additional 2 volumes of the first buffer (3 volumes total) and filtering the sample through a 40 uM filter.

25. The method of claim 24, further comprising adding an additional three volumes of the first buffer (6 volumes total), centrifuging, preferably, the sample is centrifuged at about 500 g for about 5 min, and resuspending the sample in a second buffer comprising a buffering agent and an ionic strength between 100 mM and 200 mM, optionally the second buffer comprises protease inhibitors.

26. The method of claim 25, wherein the second buffer is ST, optionally comprising protease inhibitors.

27. The method of any of the preceding claims, further comprising reversing cross-linking in the tissue sample before or during any step of the method.

28. The method of claim 27, wherein reversing cross-linking comprises proteinase digestion.

29. The method of claim 28, wherein the proteinase is proteinase K or a cold-active protease.

30. The method of any of the preceding claims, further comprising adding a reagent that stabilizes RNA to the tissue sample before or during any step of the method.

31. The method of any of the preceding claims, further comprising lysing recovered cells or nuclei and performing reverse transcription.

32. The method of claim 31, wherein the reverse transcription is performed in individual reaction vessels.

33. The method of claim 31, wherein the reaction vessels are wells, chambers, or droplets.

34. The method of any of the preceding claims, further comprising performing single cell, single nucleus or bulk RNA-seq, DNA-seq, ATAC-seq, or ChIP on the recovered nuclei or whole cells.

35. The method of any of the preceding claims, further comprising staining the recovered cells or nuclei.

36. The method of claim 35, wherein the stain comprises ruby stain.

37. A method of recovering nuclei and attached ribosomes from a tissue sample comprising:
a. chopping the tissue sample at between 0-4° C. in a nuclear extraction buffer comprising Tris buffer, a detergent and salts; and
b. filtering the sample through a filter between 30-50 uM, preferably 40 uM, and optionally washing the filter with fresh nuclear extraction buffer,
wherein the nuclei are present in the supernatant passed through the filter.

38. The method of claim 37, wherein the nuclear extraction buffer comprises 10-20 mM Tris, about 0.49% CHAPS, a salt concentration having an ionic strength of 100-250 mM, and about 0.01% BSA, whereby nuclei are recovered that have a preserved nuclear envelope and ribosomes.

39. The method of claim 38, wherein the nuclear extraction buffer is buffer CST.

40. The method of claim 37, wherein the nuclear extraction buffer comprises 10-20 mM Tris, about 0.03% Tween-20, a salt concentration having an ionic strength of 100-250 mM, and about 0.01% BSA, whereby nuclei are recovered that have a preserved nuclear envelope, rough ER and ribosomes.

41. The method of claim 40, wherein the nuclear extraction buffer is buffer TST.

42. The method of any of claims 37 to 41, wherein the salts comprise 146 mM NaCl, 1 mM CaCl₂, and 21 mM MgCl₂.

43. The method of any of claims 37 to 42, wherein chopping comprises chopping with scissors for 1-10 minutes.

44. The method of any of claims 37 to 43, wherein nuclei from specific cell types are genetically modified to express a detectable label on the nuclear membrane and the method further comprises enriching nuclei from the specific cell types using the detectable label.

45. The method of any of claims 37 to 44, further comprising staining the recovered nuclei.

46. The method of claim 45, wherein the stain comprises ruby stain.

47. The method of any of claims 37 to 46, wherein the nuclei are sorted into discrete volumes by FACS.

48. The method of any of claims 37 to 46, further comprising pelleting the nuclei and resuspending the nuclei in a second buffer consisting of Tris buffer and salts.

49. The method of claim 48, wherein the second buffer is buffer ST.

50. The method of any of claims 37 to 49, further comprising generating a single nuclei barcoded library for the recovered nuclei, wherein the nucleic acid from each nuclei is labeled with a barcode sequence comprising a cell of origin barcode, optionally the barcode sequence includes a cell of origin barcode and a unique molecular identifier (UMI).

51. The method of claim 50, wherein RNA and/or DNA is labeled with the barcode sequence.

52. The method of claim 51, wherein the library is an RNA-seq, DNA-seq, and/or ATAC-seq library.

53. The method of any of claims 50 to 52, further comprising sequencing the library.

54. The method of any of claims 37 to 53, wherein the tissue sample is fresh frozen.

55. The method of any of claims 37 to 54, wherein the tissue sample comprises cells originating from the central nervous system (CNS) or enteric nervous system (ENS).

56. The method of any of claims 37 to 55, wherein the tissue sample is obtained from the gut or the brain.

57. The method of any of claims 37 to 56, wherein the tissue sample is obtained from a subject suffering from a disease.

58. The method of any of claims 37 to 57, wherein the tissue sample is treated with a reagent that stabilizes RNA.

59. The method of any of claims 47 to 58, wherein the discrete volumes are droplets, wells in a plate, or microfluidic chambers.

60. A method of treating a disease selected from the group consisting of Hirschsprung's disease (HSCR), inflammatory bowel disease (IBD), autism spectrum disorder (ASD), Parkinson's disease (PD) and schizophrenia in a subject in need thereof comprising administering one or more agents capable of modulating the function or activity of:
a) one or more neurons selected from the group consisting of PEMN1, PEMN2, PIMN1, PIMN2, PIMN3, PIMN4, PIMN5, PIN1, PIN2, PSN and PSVN; or
b) one or more cells functionally interacting with the one or more neurons.

61. The method of claim 60, wherein the one or more cells functionally interacting with the one or more neurons are selected from the group consisting of T cells, dendritic cells (DC), B cells, fibroblasts and adipocytes.

62. A method of modulating appetite and energy metabolism in a subject in need thereof comprising administering one or more agents capable of modulating the function or activity of:
a) one or more neurons selected from the group consisting of PIMN4 and PIMN5; or
b) one or more adipose cells functionally interacting with the one or more neurons.

63. The method of any of claims 60 to 62, wherein the one or more neurons are characterized by expression of one or more markers according to Table 14 or Table 21.

64. The method of any of claims 60 to 63, wherein the one or more agents modulate the expression, activity or function of one or more genes according to Table 14 or Table 21.

65. The method of any of claim 60, 61, 63 or 64, wherein the one or more agents modulate the expression, activity or function of one or more genes selected from the group consisting of:
a) NPY, CGRP, Glutamate, GABA, LEP, VIP, PACAP, Nitric oxide, NOS1, FGF1, PDGF, SLIT2, SLIT3, IL15, IL7, IL12A, PENK, CHAT and TPH2; or
b) NPYR1, CALCRL, GRM8, GABRE, LEPR, VIPR2, GRIA4, GUCY1A3, FGFR1, PDGFRB, ROBO1, ROBO2, IL15R, IL7R, IL12RB1, OPRM1, CHRNE and HTR3A.

66. The method of claim 62, wherein the one or more agents modulate the expression, activity or function of one or more genes selected from the group consisting of:
c) NPY and CGRP; or
d) NPYR1 and CALCRL.

67. The method of any of claims 60 to 66, wherein the one or more agents modulate the expression, activity or function of one or more core transcriptional programs according to Table 23.

68. The method of claim 67, wherein the one or more agents modulate the expression, activity or function of one or more genes of the one or more core transcriptional programs.

69. The method of any of claims 60 to 68, wherein the one or more agents are administered to the gut.

70. The method of any of claims 60 to 69, wherein the one or more agents comprise an antibody, small molecule, small molecule degrader, genetic modifying agent, nucleic acid agent, antibody-like protein scaffold, aptamer, protein, or any combination thereof.

71. The method of claim 70, wherein the genetic modifying agent comprises a CRISPR system, RNAi system, a zinc finger nuclease system, a TALE, or a meganuclease.

72. The method of claim 71, wherein the CRISPR system comprises Cas9, Cas12, or Cas14.

73. The method of claim 71, wherein the CRISPR system comprises a dCas fused or otherwise linked to a nucleotide deaminase.

74. The method of claim 73, wherein the nucleotide deaminase is a cytidine deaminase or an adenosine deaminase.

75. The method of claim 73, wherein the dCas is a dCas9, dCas12, dCas13, or dCas14.

76. The method of claim 70, wherein the nucleic acid agent or genetic modifying agent is administered with a vector.

77. The method of claim 76, wherein the nucleic acid agent or genetic modifying agent is under the control of a promoter specific to a marker gene for the one or more neurons according to Table 14 or Table 21.

78. A method of detecting one or more cells of the enteric nervous system (ENS) comprising detecting one or more markers according to Table 14-17 or Table 20-22.

79. The method of claim 78, wherein detecting the one or more markers comprises immunohistochemistry.

80. A method of screening for agents capable of modulating expression of a transcription program according to Table 23 comprising:
a) administering an agent to a population of cells comprising neurons selected from the group consisting of PEMN1, PEMN2, PIMN1, PIMN2, PIMN3, PIMN4, PIMN5, PIN1, PIN2, PSN and PSVN; and
b) detecting expression of one or more genes in the transcriptional program.

81. The method of claim 80, wherein detecting expression comprises RT-PCR, RNA-seq, single cell RNA-seq, fluorescently labeled probes, or an immunoassay.

82. The method of claim 80, wherein the neurons express one or more reporter genes under control of a promoter specific to the one or more genes in the transcriptional program and detecting comprises detecting the reporter gene.

83. A method of identifying gene expression in single cells comprising providing sequencing reads from a single nucleus sequencing library and counting sequencing reads mapping to introns and exons.

84. The method of claim 83, further comprising filtering the single nuclei.

85. The method of claim 84, wherein nuclei doublets are removed by filtering.

86. The method of claim 84, wherein nuclei containing ambient RNA or ambient RNA alone is removed by filtering.


SEQUENCE LISTING (2 sequences)

SEQ ID NO: 1 - protein, 288 aa, Artificial Sequence
  Synthetic Peptide
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic Peptide
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS